SEQUENCE LISTING (12 sequences)

SEQ ID NO: 1 - protein, 8 aa, synthetic construct
GFVFSSYD

SEQ ID NO: 2 - protein, 8 aa, synthetic construct
ISSGGGIT

SEQ ID NO: 3 - protein, 13 aa, synthetic construct
AAHYFGSSGPFAY

SEQ ID NO: 4 - protein, 6 aa, synthetic construct
ENIFSY

SEQ ID NO: 5 - skipped

SEQ ID NO: 6 - protein, 9 aa, synthetic construct
QHHYGTPFT

SEQ ID NO: 7 - protein, 120 aa, synthetic construct [sequence omitted]

SEQ ID NO: 8 - protein, 107 aa, synthetic construct [sequence omitted]

SEQ ID NO: 9 - protein, 449 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 12 - protein, 654 aa, synthetic construct
QLTIESRPFNVAEGKEVLLLAHNVSQNLFGYIWYKGERVDASRRIGSCVIRTQQITPGPAHSGRETIDFNASLLIQNVTQSDTGSYTIQVIKEDLVNEEATGQFRVYPELPKPYITSNNSNPIEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGEYLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQRLSVSSRLELSNDNRTLTVFNIPRNDTTFYECETQNPVSVRRSDPVTLNVLYGPDAPTISPLNTPYRAGENLNLSCHAASNPAAQYFWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNNGAYACFVSNLATGRNNSIVKNISVSSGDSAPGSSGLSA

(19) United States
(12) Patent Application Publication
(10) Pub. No.: US 2024/0226313 A1
(43) Pub. Date: Jul. 11, 2024

(54) CEACAM5 ANTIBODY-DRUG CONJUGATES AND METHODS OF USE THEREOF

(71) Applicants: Sanofi, Paris (FR); Seagen Inc., Bothell, WA (US)

(72) Inventors: Yves BAUDAT, Paris (FR); Marie-Priscille BRUN, Paris (FR); Stéphanie DÉCARY, Paris (FR); Scott JEFFREY, Snohomish, WA (US); Ryan LYSKI, Lynnwood, WA (US); Céline NICOLAZZI, Paris (FR)

(21) Appl. No.: 18/511,818

(22) Filed: Nov. 16, 2023

Related U.S. Application Data
(60) Provisional application No. 63/384,214, filed on Nov. 17, 2022.
(60) Provisional application No. 63/596,943, filed on Nov. 7, 2023.

(30) Foreign Application Priority Data
Dec. 2, 2022 (EP) 22306780.2

(51) Int. Cl. A61K 47/68; A61P 35/00; C07K 16/30

(52) U.S. Cl. CPC A61K 47/68037; A61K 47/6863; A61P 35/00; C07K 16/3007

(57) ABSTRACT

The present invention relates to antibody-drug conjugates comprising an antibody that binds to CEACAM5 conjugated to a drug, such as a Topoisomerase I inhibitor. Also provided herein are methods of treating cancer comprising administering such ADCs.

### CROSS-REFERENCE TO RELATED APPLICATIONS

This application claims priority benefit of U.S. Provisional Patent Application No. 63/384,214, filed Nov. 17, 2022, European Patent Application No. 22306780.2, filed Dec. 2, 2022, and U.S. Provisional Patent Application No. 63/596,943, filed Nov. 7, 2023, the disclosures of which are hereby incorporated herein by reference in their entirety.

### REFERENCE TO AN ELECTRONIC SEQUENCE LISTING

The content of the electronic sequence listing (761682009900seglist.xml; Size: 12,724 bytes; and Date of Creation: Nov. 15, 2023) is herein incorporated by reference in its entirety.

### TECHNICAL FIELD

This application relates to antibody-conjugates (ADC) comprising an antibody that binds to CEACAM5 conjugated to a Topoisomerase I inhibitor.

### BACKGROUND

Carcino-embryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7, CEACAM8. Numerous studies have shown that CEACAM5 is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarström et al, 2002, in “Tumor markers, Physiology, Pathobiology, Technology and Clinical Applications” Eds. Diamandis E. P. et al., AACC Press, Washington pp 375). Thus, CEACAM5 may constitute a therapeutic target suitable for tumor specific targeting approaches, such as immunoconjugates. The present invention provides monoclonal antibodies directed against CEACAM5, and shows that they can be conjugated to a cytotoxic agent to induce a cytotoxic activity able to kill tumor cells in vitro and to induce tumor regression in vivo.

The extracellular domains of CEACAM family members are composed of repeated immunoglobulin-like (Ig-like) domains which have been categorized in 3 types, A, B and N, according to sequence homologies. CEACAM5 contains seven such domains, namely N, A1, B1, A2, B2, A3 and B3.

CEACAM5 A1, A2 and A3 domains, on one hand, and B1, B2 and B3 domains, on the other hand, show high sequence homologies, the A domains of human CEACAM5 presenting from 84 to 87% pairwise sequence similarity, and the B domains from 69 to 80%. Furthermore, other human CEACAM members presenting A and/or B domains in their structure, namely CEACAM1, CEACAM6, CEACAM7 and CEACAM8, show homology with human CEACAM5. In particular, the A and B domains of human CEACAM6 protein display sequence homologies with A1 and A3 domains, and any of B1 to B3 domains of human CEACAM5, respectively, which are even higher than observed among the A domains and the B domains of human CEACAM5.

The design of Antibody Drug Conjugates (ADCs), by attaching a cytotoxic agent to antibody, typically via a linker, involves consideration of a variety of factors, including the presence of a conjugation handle on the drug for attachment to the linker and linker technology for attaching the drug to an antibody in a conditionally stable manner. Thus, one strategy for cancer therapies targeting CEACAM5 is by producing an ADC comprising an antibody that binds to CEACAM5 conjugated to a cytotoxic drug.

### SUMMARY

In some aspects, provided herein is an antibody-drug conjugate that binds to CEACAM5 having the formula of L-(Q-D)ₚ, or a salt thereof, wherein:

L is a Ligand Unit comprising an antibody or antigen binding fragment thereof that binds to CEACAM5;

subscript p is an integer ranging from 1 to 16;

Q is a Linker Unit having a formula selected from the group consisting of:
—Z-A-RL-, —Z-A-RL-Y—, —Z-A-S*-RL-, —Z-A-B(S*)—RL-, —Z-A-S*-RL-Y—, and —Z-A-B(S*)—RL-Y—;

wherein Z is a Stretcher Unit;

A is a bond or a Connector Unit;

B is a Parallel Connector Unit;

S* is a Partitioning Agent;

RL is a glycoside unit;

Y is a Spacer Unit; and

D is a Drug Unit having the formula of:

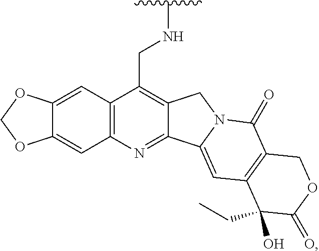 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q.

In some aspects, provided herein is an antibody-drug conjugate that binds to CEACAM5 having the formula of L-(Q-D)ₚ, or a salt thereof, wherein

L is a Ligand Unit comprising an antibody or antigen binding fragment thereof that binds to CEACAM5 comprising

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8;

subscript p is an integer ranging from 1 to 16;

Q is a Linker Unit;

and D is a Drug Unit, wherein the Drug Unit is a Topoisomerase I inhibitor.

In some embodiments, the antibody or antigen binding fragment thereof comprises

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8.

In some embodiments of the foregoing antibody-drug conjugate or salt thereof, the antibody or antigen binding fragment thereof comprises a variable heavy chain domain (VH) that has at least 80%, 85%, 90%, 95% or 99% amino acid sequence identity to the amino acid sequence of SEQ ID NO: 7 and wherein a variable light chain domain (VL) that has at least 80%, 85%, 90%, 95% or 99% amino acid sequence identity to the amino acid sequence of SEQ ID NO: 8.

In some embodiments, provided herein is an antibody-drug conjugate or salt thereof of, wherein the antibody or antigen binding fragment thereof comprises

a CDR1-H comprising the amino acid sequence set forth in SEQ ID NO:1

a CDR2-H comprising the amino acid sequence set forth in SEQ ID NO:2;

a CDR3-H comprising the amino acid sequence set forth in SEQ ID NO:3;

a CDR1-L comprising the amino acid sequence set forth in SEQ ID NO:4;

a CDR2-L comprising the amino acid sequence NTR; and

a CDR3-L comprising the amino acid sequence set forth in SEQ ID NO:6.

In some embodiments, provided herein is an antibody-drug conjugate or salt thereof of, wherein the antibody or antigen binding fragment thereof comprises a heavy chain that has at least 80%, 85%, 90%, 95% or 99% amino acid sequence identity to the amino acid sequence set forth in SEQ ID NO:9, and a light chain that has at least 80%, 85%, 90%, 95% or 99% amino acid sequence identity to the amino acid sequence set forth in SEQ ID NO:10.

In some embodiments, provided herein is an antibody-drug conjugate or salt thereof of, wherein the antibody or antigen binding fragment thereof is chimeric or humanized.

In some embodiments, provided herein is an antibody-drug conjugate or salt thereof of, wherein the antibody or antigen binding fragment is selected from the group consisting of a of Fv, Fab, F(ab′)₂, Fab′, dsFv, (dsFv)₂, scFv, sc(Fv)₂, and a diabody.

In some embodiments, Q is a Linker Unit having the formula —Z-A-RL-. In some embodiments, RL is a Glucuronide Unit.

In some embodiments, RL is a Glucuronide Unit having the formula:

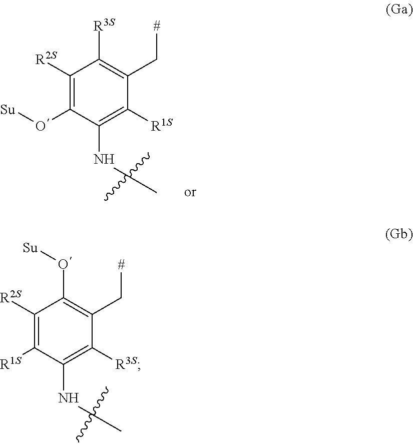

wherein
Su is a sugar moiety;
—O′— is an oxygen glycosidic bond;
R^{1S}, R^{2S} and R^{3S} independently are hydrogen, halogen, —CN, —NO₂, or other electron withdrawing group, or an electron donating group;
the wavy line indicates attachment to Z, either directly or indirectly through A or B or A and B; and
# indicates attachment to D or Y, either directly or indirectly via an intervening functional group or other moiety.

In some embodiments, RL is a Glucuronide Unit having the formula:

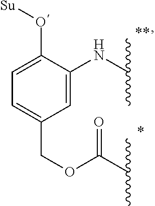

wherein
Su is a sugar moiety;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

In some embodiments, Su is a hexose form of a monosaccharide.

In some embodiments, the Glucuronide Unit has the formula:

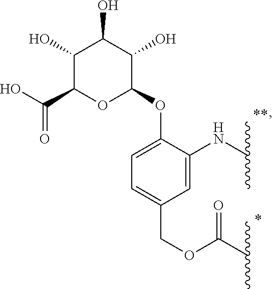 [SMILES: *Nc1cc(COC(*)=O)ccc1O[C@H]1O[C@@H](C(=O)O)[C@H](O)[C@@H](O)[C@@H]1O]

wherein the wavy line marked with a single asterisk (*) indicates the site of covalent attachment to D; and the wavy line marked with a double asterisk (**) indicates the site of covalent attachment to the remainder of Q.

In some embodiments, Z comprises a succinimido-alkanoyl moiety, optionally having the succinimide ring in hydrolyzed form as a succinic acid amide moiety.

In some embodiments, Z is

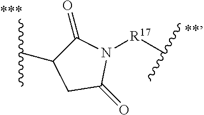

optionally having the succinimide ring in hydrolyzed form as a succinic acid amide moiety, wherein:

the wavy line marked with a double asterisk (**) indicates the site of covalent attachment to the rest of Q;

the wavy line marked with a triple asterisk (***) indicates the point of covalent attachment to a sulfur atom of L; and

R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In some embodiments, R¹⁷ is —(CH₂)₂₋₅—C(═O)—.

In some embodiments, Z is

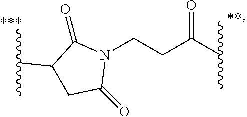 [SMILES: *C(=O)CCN1C(=O)CC(*)C1=O]

optionally having the succinimide ring in hydrolyzed form as a succinic acid amide moiety, wherein the wavy line marked with a double asterisk (**) indicates the site of covalent attachment to the rest of Q; and the wavy line marked with a triple asterisk (***) indicates the point of covalent attachment to a sulfur atom of L.

In some embodiments, Z is

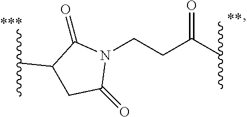 [SMILES: *C(=O)CCN1C(=O)CC(*)C1=O]

wherein the wavy line marked with a double asterisk (**) indicates the site of covalent attachment to the rest of Q; and the wavy line marked with a triple asterisk (***) indicates the point of covalent attachment to a sulfur atom of L.

In some embodiments, Z is

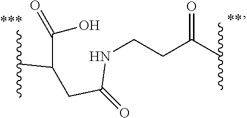 [SMILES: *C(=O)CCNC(=O)CC(*)C(=O)O]

wherein the wavy line marked with a double asterisk (**) indicates the site of covalent attachment to the rest of Q; and the wavy line marked with a triple asterisk (***) indicates the point of covalent attachment to a sulfur atom of L.

In some embodiments, Z is

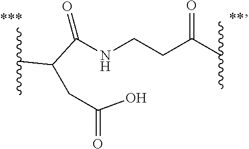 [SMILES: *C(=O)CCNC(=O)C(*)CC(=O)O]

wherein the wavy line marked with a double asterisk (**) indicates the site of covalent attachment to the rest of Q; and the wavy line marked with a triple asterisk (***) indicates the point of covalent attachment to a sulfur atom of L.

In some embodiments, A is a Connector Unit.

In some embodiments, A has the formula:

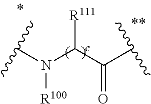

wherein the wavy line marked with a double asterisk (**) indicates the site of covalent attachment to RL;

the wavy line marked with a single asterisk (*) indicates the point of covalent attachment to Z;

R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

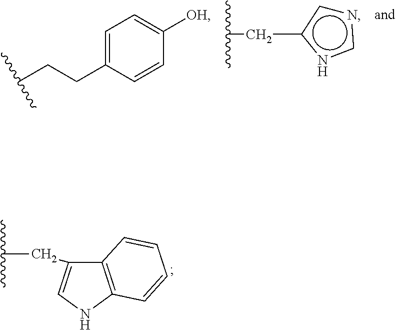

each R¹⁰⁰ is independently selected from the group consisting of hydrogen and —C₁-C₃ alkyl; and

c is an independently selected integer from 1 to 10.

In some embodiments, A has the formula:

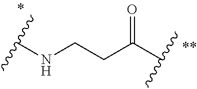 [SMILES: *NCCC(*)=O]

wherein the wavy line marked with a double asterisk (**) indicates the site of covalent attachment to RL; and the wavy line marked with a single asterisk (*) indicates the point of covalent attachment to Z.

In some embodiments, —Z-A- has the formula:

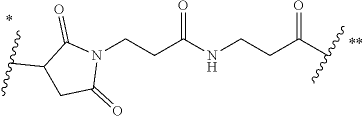 [SMILES: *C(=O)CCNC(=O)CCN1C(=O)CC(*)C1=O]

wherein the wavy line marked with a double asterisk (**) indicates the site of covalent attachment to RL; and the wavy line marked with a single asterisk (*) indicates the point of covalent attachment to L.

In some embodiments, —Z-A-RL- has the formula:

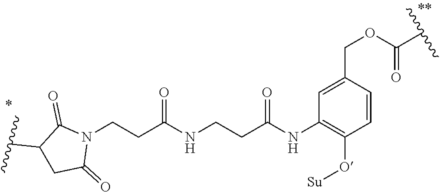

wherein Su is a hexose form of a monosaccharide; O′ represents the oxygen atom of a glycosidic bond that is capable of cleavage by a glycosidase; the wavy line marked with a double asterisk (**) indicates the site of covalent attachment to D; and the wavy line marked with a single asterisk (*) indicates the point of covalent attachment to L.

In some embodiments, —Z-A-RL- has the formula:

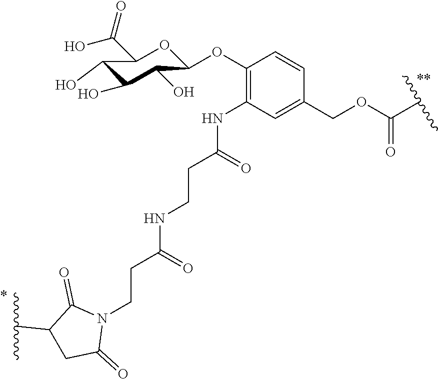

wherein the wavy line marked with a double asterisk (**) indicates the site of covalent attachment to D; and the wavy line marked with a single asterisk (*) indicates the point of covalent attachment to L.

In some embodiments, —RL-D- has the formula:

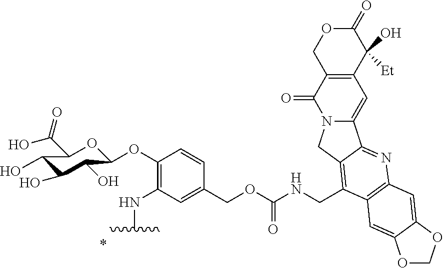

wherein the wavy line marked with a single asterisk (*) indicates the point of covalent attachment to A.

In some embodiments, -A-RL-D has the formula:

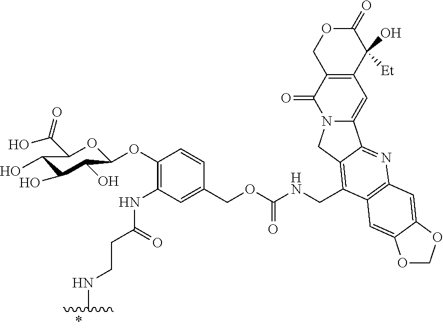

Wherein the wavy line marked with a single asterisk (*) indicates the point of covalent attachment to Z.

In some embodiments, S* is a PEG group.

In some embodiments, -Q-D- has the formula:

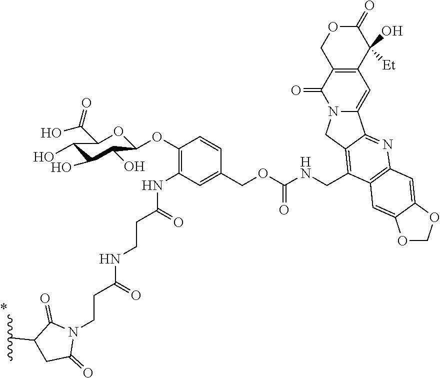

Wherein the wavy line marked with a single asterisk (*) indicates the point of covalent attachment to L.

In some embodiments, -Q-D- has the formula:

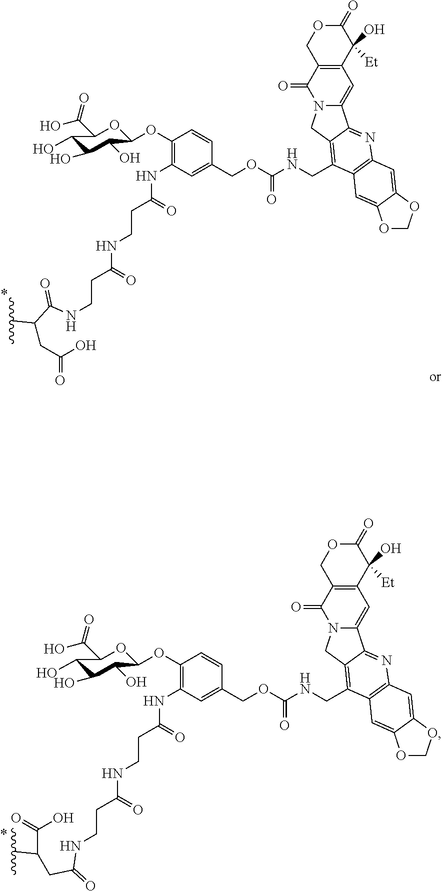

Wherein the wavy line marked with a single asterisk (*) indicates the point of covalent attachment to L.

In some embodiments, the Drug Unit is a Topoisomerase I inhibitor.

In some embodiments, which can be combined with any of the embodiments described above or below, provided herein is an antibody-drug conjugate or salt thereof of, comprising a ratio of Drug Unit to antibody (DAR) ratio of 1 to 10.

In some embodiments, the DAR is about 4 or about 8.

In some embodiments, p is an integer of about 1 to about 10.

In some embodiments, p is an integer of about 4 or about 8.

In some embodiments, the Linker Unit is attached to the antibody or antigen binding fragment at a cysteine amino acid residue. In some embodiments, the cysteine is a native cysteine. In some embodiments, the cysteine is located in hinge region of the antibody or antigen-binding fragment thereof. In some embodiments, the antibody or antigen binding fragment thereof is cysteine engineered.

In some aspects, provided herein is a pharmaceutical composition comprising the antibody-drug conjugate or salt thereof described herein, and a pharmaceutically acceptable carrier.

In some aspects, provided herein is a method of treating cancer in an individual comprising administering the antibody-drug conjugate or salt thereof or the pharmaceutical composition described herein to the individual.

In some aspects, provided herein is an antibody-drug conjugate or salt thereof or the pharmaceutical composition described herein, for use in the treatment of cancer.

In some embodiments, the cancer is a solid tumor.

In some embodiments, the cancer is selected from the group consisting of colorectal cancer, neuroendocrine cancers, stomach cancers, lung cancers, uterus cancers, cervical cancers, pancreatic cancers, esophagus cancers, ovarian cancers, thyroid cancers, bladder cancers, endometrium cancers, bladder cancers, endometrial cancers, breast cancers, liver cancers, prostate cancers, gastric cancers, cholangiocarcinoma and skin cancers.

In some embodiments, the cancer is selected from the group consisting of colorectal cancer, stomach cancers, gastric cancer, Gastroesophageal Junction cancer, lung cancers, uterus cancers, cervical cancers, pancreatic cancers, esophagus cancers, ovarian cancers, thyroid cancers, bladder cancers, endometrium cancers, bladder cancers, neuroendocrine cancers, endometrial cancers, breast cancers, liver cancers, prostate cancers, and cholangiocarcinoma and skin cancers. In some embodiments, the lung cancers include Non-Small-Cell-Lung Carcinoma (NSCLC), non-squamous-NSCLC (nsq-NSCLC), squamous-NSCLC (sq-NSCLC), or Small-Cell-Lung-Carcinoma (SCLC)), or any combination thereof. In some embodiments, the pancreatic cancers include Pancreatic Ductal Adenocarcinoma (PDAC).

In some embodiments, the cancer is selected from the group consisting of colorectal cancer, lung cancer, gastric cancer, and pancreatic cancer.

In some embodiments, cancer is selected from the group consisting of colorectal cancer, lung cancers, gastric cancers, Gastroesophageal Junction cancers, neuro endocrine cancers and pancreatic cancers.

In some embodiments, the cancer is colorectal cancer, NSCLC, SCLC, gastric cancers, gastroesophageal Junction cancer and Pancreatic Ductal Adenocarcinoma.

In some embodiments, the cancer is primary, metastatic or carcinosis.

In some embodiments, the tumor expresses a high level CEACAM5. In some embodiments, at least 50% of tumor cells in a sample of the tumor score a greater than 2+ intensity as measured by immunohistochemistry.

In some embodiments, the tumor expresses a moderate level CEACAM5. In some embodiments, at least 1% and less than 50% of tumor cells in a sample of the tumor score a ≥2+ intensity as measured by immunohistochemistry or at least 50% of tumor cells in a sample of the tumor score a 1+ intensity as measured by immunohistochemistry.

In some embodiments, the tumor expresses any level of CEACAM5. In some embodiments, reactivity for CEACAM5 is observed but the CEACAM5 expression level is not considered moderate or high.

In some embodiments, the antibody-drug conjugate or salt thereof does not induce a significant level of toxicity in the individual.

In some embodiments, the antibody-drug conjugate or salt thereof causes a reduction in tumor volume following administration.

In some aspects, provided herein is a kit comprising an antibody-drug conjugate or salt thereof or the pharmaceutical composition described herein.

### BRIEF DESCRIPTION OF THE DRAWINGS

FIGS. 1A and 1B show in vitro binding ability of ADC1 with a DAR of 8 to huFcRn expressed at the cell surface of HEK293 cells under pH 7.2 and pH 6.6, respectively.

FIG. 2 shows evaluation of the anti-tumor activity (e.g., reduction of tumor volume) of ADC1 against CRC patient-derived xenograft tumor CR-IGR-0002P in SCID female mice. The curves represent medians+ or − MAD at each day for each group. The black arrow indicates the days of treatment (single administration).

FIG. 3 shows evaluation of the anti-tumor activity (e.g., reduction of tumor volume) of ADC1 against CRC patient-derived xenograft tumor, CR-IGR-0007P in SCID female mice. The curves represent medians+ or − MAD at each day for each group. The black arrow indicates the days of treatment (single administration).

FIG. 4 shows evaluation of the anti-tumor activity (e.g., reduction of tumor volume) of ADC1 against CRC patient-derived xenograft tumor, CR-IGR-0048M in SCID female mice. The curves represent medians+ or − MAD at each day for each group. The black arrow indicates the days of treatment (single administration).

FIG. 5 shows evaluation of the anti-tumor activity (e.g., reduction of tumor volume) of ADC1 against CRC patient-derived xenograft tumor, CR-IC-0016M in SCID female mice. The curves represent medians+ or − MAD at each day for each group. The black arrow indicates the days of treatment (single administration).

FIG. 6 shows evaluation of the anti-tumor activity (e.g., reduction of tumor volume) of ADC1 against lung patient-derived xenograft tumor, LUN-NIC-0014 in SCID female mice. The curves represent medians+ or − MAD at each day for each group. The black arrow indicates the days of treatment (single administration).

FIG. 7 shows evaluation of the anti-tumor activity (e.g., reduction of tumor volume) of ADC1 against lung patient-derived xenograft tumor, LUN-NIC-0084 in SCID female mice. The curves represent medians+ or − MAD at each day for each group. The black arrow indicates the days of treatment (single administration).

FIG. 8 shows evaluation of the anti-tumor activity (e.g., reduction of tumor volume) of ADC1 against lung patient-derived xenograft tumor, LUN-NIC-0004 in SCID female mice. The curves represent medians+ or − MAD at each day for each group. The black arrow indicates the days of treatment (single administration).

FIG. 9 shows evaluation of the anti-tumor activity (e.g., reduction of tumor volume) of ADC1 against lung patient-derived xenograft tumor, LUN-NIC-0008 in SCID female mice. The curves represent medians+ or − MAD at each day for each group. The black arrow indicates the days of treatment (single administration).

FIG. 10 shows evaluation of the anti-tumor activity (e.g., reduction of tumor volume) of ADC1 against gastric patient-derived xenograft tumor, STO-IND-0006 in SCID female mice. The curves represent medians+ or − MAD at each day for each group. The black arrow indicates the days of treatment (single administration).

FIG. 11 shows evaluation of the anti-tumor activity (e.g., reduction of tumor volume) of ADC1 against gastric patient-derived xenograft tumor, SA-STO-0014 in SCID female mice. The curves represent medians+ or − MAD at each day for each group. The black arrow indicates the days of treatment (single administration).

FIG. 12 shows evaluation of the anti-tumor activity (e.g., reduction of tumor volume) of ADC1 against gastric patient-derived xenograft tumor, STO-IND-0007 in SCID female mice. The curves represent medians+ or − MAD at each day for each group. The black arrow indicates the days of treatment (single administration).

FIG. 13 shows evaluation of the anti-tumor activity of ADC1 in a panel of 16 colon patient-derived xenograft models under Single Mouse Trial—Best relative tumor shrinkage. The best relative tumor shrinkage or best response to ADC1 was represented under a waterfall plot. PDX models are sorted by increasing sensitivity to ADC1.

FIG. 14 shows in vivo efficacy evaluation of ADC1 in a panel of 19 gastric patient-derived xenograft models under Single Mouse Trial format. The best relative tumor shrinkage or best response to ADC1 was represented under a waterfall plot. PDX models are sorted by increasing sensitivity to ADC1.

FIG. 15 shows in vivo efficacy evaluation of ADC1 in a panel of 31 lung patient-derived xenograft models under Single Mouse Trial format. The best relative tumor shrinkage or best response to ADC1 was represented under a waterfall plot. PDX models are sorted by increasing sensitivity to ADC1.

FIG. 16 shows in vivo efficacy evaluation of ADC1 against pancreas patient-derived xenograft tumor IM-PAN-011 (metastatic PDAC) implanted subcutaneously in female SCID mice. Tumor volume evolution by treatment group. The curves represent medians+ or − MAD at each day for each group. The black arrow indicates the days of treatment (single administration).

FIG. 17 shows in vivo efficacy evaluation of ADC1 against pancreas patient-derived xenograft tumor SA-PAN-0077 (PDAC) implanted subcutaneously in female SCID mice. Tumor volume evolution by treatment group. The curves represent medians+ or -MAD at each day for each group. The black arrow indicates the days of treatment (single administration).

FIG. 18 shows in vivo efficacy evaluation of ADC1 against pancreas patient-derived xenograft tumor IM-PAN-0006 (PDAC) implanted subcutaneously in female SCID mice. Tumor volume evolution by treatment group. The curves represent medians+ or -MAD at each day for each group. The black arrow indicates the days of treatment (single administration).

FIG. 19 shows in vivo efficacy evaluation of ADC1 against pancreas patient-derived xenograft tumor IM-PAN-003 (PDAC) implanted subcutaneously in female SCID mice. Tumor volume evolution by treatment group. The curves represent medians+ or -MAD at each day for each group. The black arrow indicates the days of treatment (single administration).

FIG. 20 shows total CD PK and DAR profiles in plasma following single intravenous administration of ADC1 with a DAR of 8 to SCID mice at 3 mg/kg.

### DETAILED DESCRIPTION

In some embodiments, provided herein are antibody drug conjugates (ADC) comprising an antibody or antigen binding fragment thereof that bind to CEACAM5 conjugated to a Topoisomerase I inhibitor. In some embodiments, the ADC comprises an antibody or antigen binding fragment thereof that binds to CEACAM5 conjugated to a camptothecin. In some embodiments, advantageously the ADCs bind to CEACAM5 at nanomolar concentrations and kill different CEACAM5-positive CRC cells at sub nanomolar concentrations with low toxicity towards CEACAM5 negative cells. Without wishing to be bound by theory, the cytotoxicity of the ADC provided herein may be mediated by its internalization, processing, and cytotoxic release in the CEACAM5-expressing tumor cells. Additionally, or alternatively, the cytotoxicity may be mediated by a bystander effect allowing the cytotoxic agent to diffuse to neighboring CEACAM5 negative tumor cells, resulting in cell killing. In some embodiments, the bystander effect allows the payload to diffuse from antigen-positive tumor cells to adjacent antigen-negative tumor cells, resulting in cell killing. In some embodiments, the present ADCs are well tolerated with significant antitumor activity, particularly in colorectal cancer (CRC) cell models. In some embodiments, the present ADCs are well tolerated with significant anti-tumor activity, particularly in colorectal cancer (CRC), gastric cancer (GC), gastroesophageal junction cancer (GEJ), lung cancers and pancreatic cancers tumor models. In some embodiments, the topoisomerase I payload was optimized for potency, reduced P-gp efflux and enhanced bystander activity.

The section headings used herein are for organizational purposes only and are not to be construed as limiting the subject matter described.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 4^{th} edition (2012) Cold Spring Harbor Laboratory Press, Cold Spring Harbor N.Y.; Current Protocols In Molecular Biology (F. M. Ausubel, et al. eds., (2003)); the series METHODS IN ENZYMOLOGY (Academic Press, Inc.); PCR 2: A Practical Approach (M. J. MacPherson, B. D. Hames and G. R. Taylor eds. (1995)); Greenfield, ed. (2013) Antibodies, A Laboratory Manual, 2^{nd} edition, Cold Spring Harbor Laboratory Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); Cancer: Principles and Practice of Oncology (V. T. DeVita et al., eds., J.B. Lippincott Company, 1993); and updated versions thereof. Each of the foregoing references in this paragraph is incorporated herein by reference in its entirety.

### I. Definitions

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 5^{th} ed., 2013, Academic Press; and the Oxford Dictionary Of Biochemistry And Molecular Biology, 2^{nd} ed., 2006, Oxford University Press, provide one of skill with a general dictionary of many of the terms used in this disclosure.

Unless otherwise required by context or expressly indicated, singular terms shall include pluralities and plural terms shall include the singular.

It is understood that aspect and embodiments of the invention described herein include “comprising”, “consisting”, and/or “consisting essentially of” aspects and embodiments.

As used herein, the singular form “a” “an”, and “the” should be understood to refer to “one or more” of any recited or enumerated component unless indicated otherwise.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” refers to a value or composition that is within an acceptable error range for the particular value or composition as determined by one of ordinary skill in the art, which will depend in part on how the value or composition is measured or determined, i.e., the limitations of the measurement system. As is understood by one skilled in the art, reference to “about” a value or parameter herein includes (and describes) embodiments that are directed to that value or parameter per se. For example, description referring to “about X” includes description of “X”.

When a trade name is used herein, reference to the trade name also refers to the product formulation, the generic drug, and the active pharmaceutical ingredient(s) of the trade name product, unless otherwise indicated by context.

The terms CEACAM5 carcino-embryonic antigen-related cell adhesion molecule 5, and CD66e, are used interchangeably herein, and, unless otherwise specified, include any naturally occurring variants (e.g., splice variants, allelic variants), isoforms, and vertebrate species homologs of human CEACAM5. The term encompasses “full length,” unprocessed CEACAM5 as well as any form of CEACAM5 that results from processing within a cell. The amino acid sequence of an exemplary human CEACAM5 is provided in the GenBank database under accession number AAA51967.1 The amino acid sequence of one specific example of a mature human CEACAM5 protein is set forth in SEQ ID NO:11

An “antibody” may be a natural or conventional antibody in which two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that areprimarily from the hypervariable or complementarity determining regions (CDRs). Occasionally, residues from nonhypervariable or framework regions (FR) influence the overall domain structure and hence the combining site. Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively.

As used herein, a “human framework region” is a framework region that is substantially identical (about 85%, or more, for instance 90%, 91%, 92%, 93%, 94%, 95%, 96%, 97%, 98% or 99%) to the framework region of a naturally occurring human antibody. In the context of the invention, CDR/FR definition in an immunoglobulin light or heavy chain is to be determined based on IMGT definition (Lefranc et al. Dev. Comp. Immunol., 2003, 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanized, bispecific or multispecific antibodies.

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e. produced by protein engineering.

The term “chimeric antibody” refers to an engineered antibody which, in itsbroadest sense, contains one or more regions from one antibody and one or more regions from one or more other antibodies. In an embodiment, a chimeric antibody comprises a VH domain and a VL domain of an antibody derived from a non-human animal, in association with a CH domain and a CL domain of another antibody, in an embodiment, a human antibody. As the non-human animal, any animal such as mouse, rat, hamster, rabbit or the like can be used. A chimeric antibody may also denote a multispecific antibody having specificity for at least two different antigens.

The term “humanized antibody” refers to an antibody which is wholly or partially of non-human origin and which has been modified to replace certain amino acids, for instance in the framework regions of the VH and VL domains, in order to avoid or minimize an immune response in humans. The constant domains of a humanized antibody are most of the time human CH and CL domains.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 and antigen binding activity, in which about a half of the N-terminal side of the heavy chain and the entire light chain are bound together through a disulfide bond. It is usually obtained among fragments by treating IgG with a protease, papain.

The term “F(ab′)2” refers to an antibody fragment having a molecular weight of about 100,000 and antigen binding activity, which is slightly larger than 2 identical Fab fragments bound via a disulfide bond of the hinge region. It is usually obtained among fragments by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, for instance by using gene recombination techniques. Divalent and multivalent antibody fragments can form either spontaneously by association of monovalent scFvs, or can be generated by coupling monovalent scFvs by a peptide linker, such as divalent sc(Fv)₂. “dsFv” is a VH::VL heterodimer stabilised by a disulphide bond. “(dsFv)2” denotes two dsFv coupled by a peptide linker.

The term “bispecific antibody” or “BsAb” denotes an antibody which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which combines the antigen-binding sites of two or more antibodies within a single molecule.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains of the same chain, the domains are forced to pair with the complementary domains of another chain and create two antigen-binding sites.

The term “hybridoma” denotes a cell, which is obtained by subjecting a B cell prepared by immunizing a non-human mammal with an antigen to cell fusion with a myeloma cell derived from a mouse or the like which produces a desired monoclonal antibody having an antigen specificity.

By “purified” and “isolated” it is meant, when referring to a polypeptide (i.e. the antibody of the invention) or a nucleotide sequence, that the indicated molecule is present in the substantial absence of other biological macromolecules of the same type. The term “purified” as used herein means at least 75%, 85%, 95%, 96%, 97%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

The terms “polypeptide” and “protein” are used interchangeably to refer to a polymer of amino acid residues and are not limited to a minimum length. Such polymers of amino acid residues can contain natural or non-natural amino acid residues, and include, but are not limited to, dimers, trimers, peptides, oligopeptides, and multimers of amino acid residues. Both full-length proteins and fragments thereof are encompassed by the definition. The terms also include post-expression modifications of the polypeptide, for example, glycosylation, sialylation, acetylation, phosphorylation, and the like. The term “polypeptide” also refers to a protein which includes modifications, such as deletions, additions, and substitutions (generally conservative in nature), to the native sequence, so long as the protein maintains the desired activity. The terms “polypeptide” and “protein” encompass CEACAM5 antigen binding proteins, including antibodies, antibody fragments, or sequences that have deletions from, additions to, and/or substitutions of one or more amino acids of the antigen binding protein.

A “native sequence” or a “naturally-occurring” polypeptide comprises a polypeptide having the same amino acid sequence as a polypeptide found in nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide (e.g., an antigen binding protein or antibody) having at least about 70%, 80%, or 90% amino acid sequence identity with the native or a reference sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “Percent (%) amino acid sequence identity” and “homology” with respect to a peptide, polypeptide or antigen binding protein (e.g., antibody) sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. Unless specifically stated otherwise, all % amino acid sequence identity values used herein are calculated according to this formula using the ALIGN-2 computer program. It will be appreciated that where the length of amino acid sequence A is not equal to the length of amino acid sequence B, the % sequence identity of A to B will not equal the % sequence identity of B to A.

The term “leader sequence” refers to a sequence of amino acid residues located at the N-terminus of a polypeptide that facilitates secretion of a polypeptide from a mammalian cell. A leader sequence may be cleaved upon export of the polypeptide from the mammalian cell, forming a mature protein. Leader sequences can be natural or synthetic, and they can be heterologous or homologous to the protein to which they are attached.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See, for instance, Fundamental Immunology (Paul, W., ed., 7^{th} ed. Raven Press, N.Y. (2013)). Briefly, each heavy chain typically is comprised of a heavy chain variable region (abbreviated herein as VH or VH) and a heavy chain constant regioⁿ (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

TABLE 1

| Loop | IMGT | Kabat | AbM | Chothia | Contact |
|---|---|---|---|---|---|
| CDR-H1 | 27-38 | 31-35 | 26-35 | 26-32 | 30-35 |
| CDR-H2 | 56-65 | 50-65 | 50-58 | 52-56 | 47-58 |
| CDR-H3 | 105-117 | 95-102 | 95-102 | 95-102 | 93-101 |
| CDR-L1 | 27-38 | 24-34 | 24-34 | 24-34 | 30-36 |
| CDR-L2 | 56-65 | 50-56 | 50-56 | 50-56 | 46-55 |
| CDR-L3 | 105-117 | 89-97 | 89-97 | 89-97 | 89-96 |

Unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, AbM, Chothia, or Contact method. In other instances, the particular amino acid sequence of a CDR is given.

Thus, in some embodiments, the antigen binding protein comprises CDRs and/or HVRs as defined by the IMGT system. In other embodiments, the antigen binding protein comprises CDRs or HVRs as defined by the Kabat system. In still other embodiments, the antigen binding protein comprises CDRs or HVRs as defined by the AbM system. In further embodiments, the antigen binding protein comprises CDRs or HVRs as defined by the Chothia system. In yet other embodiments, the antigen binding protein comprises CDRs or HVRs as defined by the IMGT system.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as complementarity-determining regions (CDRs), interspersed with regions that are more conserved, termed framework regions (FRs).

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an a μ constant region) antibodies; and IgM antibodies include, but are not limited to, IgM1 and IgM2.

The term “heavy chain” (HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain constant region” as used herein refers to a region comprising a light chain constant domain, C_{L}. Nonlimiting exemplary light chain constant regions include λ and κ.

The term “light chain” (LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The “EU numbering system” or “EU index” is generally used when referring to a residue in an immunoglobulin heavy chain constant region (e.g., the EU index reported in Kabat et al., Sequences of Proteins of Immunological Interest, 5^{th} Ed. Public Health Service, National Institutes of Health, Bethesda, Md., 1991). The “EU index as in Kabat” refers to the residue numbering of the human IgG1 EU antibody. Unless stated otherwise herein, references to residue numbers in the constant domain of antibodies means residue numbering by the EU numbering system.

The term “derivative” refers to a molecule (e.g., an antigen binding protein such as an antibody or fragment thereof) that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a derivative of a particular antigen binding protein can have a greater circulating half-life than an antigen binding protein that is not chemically modified. In certain embodiments, a derivative can have improved targeting capacity for desired cells, tissues, and/or organs. In some embodiments, a derivative of an antigen binding protein is covalently modified to include one or more polymers, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. See, e.g., U.S. Pat. Nos. 4,640,835, 4,496,689, 4,301,144, 4,670,417, 4,791,192 and 4,179,337.

As used herein, the term “epitope” refers to a site on an antigen (e.g., CEACAM5), to which an antigen-binding protein (e.g., an antibody or fragments thereof) that targets that antigen binds. Epitopes often consist of a chemically active surface grouping of molecules such as amino acids, polypeptides, sugar side chains, phosphoryl or sulfonyl groups, and have specific three-dimensional structural characteristics as well as specific charge characteristics. Epitopes can be formed both from contiguous or noncontiguous amino acids of the antigen that are juxtaposed by tertiary folding. Epitopes formed from contiguous residues typically are retained on exposure to denaturing solvents, whereas epitopes formed by tertiary folding typically are lost on treatment with denaturing solvents. In certain embodiments, an epitope can include, but is not limited to, at least 3, at least 4, at least 5, at least 6, at least 7, amino acids in a unique spatial arrangement. In some embodiments, the epitope refers to 3-5, 4-6, or 8-10 amino acids in a unique spatial conformation. In further embodiments, an epitope is less than 20 amino acids in length, less than 15 amino acids or less than 12 amino acids, less than 10 amino acids, or less than 8 amino acids in length. The epitope can comprise amino acids residues directly involved in the binding (also called immunodominant component of the epitope) and other amino acid residues that are not directly involved in the binding, including amino acid residues that are effectively blocked or covered by the antigen binding molecule (i.e., the amino acids are within the footprint of the antigen binding molecule). Methods of determining spatial conformation of epitopes include, for example, x-ray crystallography, two-dimensional nuclear magnetic resonance, and HDX-MS (see, e.g., Epitope Mapping Protocols in Methods in Molecular Biology, Vol. 66, G. E. Morris, Ed. (1996)). Once a desired epitope of an antigen is determined, antigen binding proteins (e.g., antibodies or fragments thereof) to that epitope can be generated using established techniques. It is then possible to screen the resulting antigen binding proteins in competition assays to identify antigen binding proteins that bind the same or overlapping epitopes. Methods for binning antibodies based upon cross-competition studies are described in WO 03/48731.

A “nonlinear epitope” or “conformational epitope” comprises noncontiguous polypeptides, amino acids, and/or sugars within the antigenic protein to which an antibody specific to the epitope binds.

A “linear epitope” comprises contiguous polypeptides, amino acids, and/or sugars within the antigenic protein to which an antigen binding protein (e.g., an antibody or fragment thereof) specific to the epitope binds.

A “paratope” or “antigen binding site” is the site on the antigen binding protein (e.g., antibody or fragment thereof) that binds the epitope and typically includes the amino acids that are in close proximity to the epitope once the antibody is bound (see, e.g., Sela-Culang et al., 2013, Front Immunol. 4:302).

“Affinity” refers to the strength of the sum total of noncovalent interactions between a single binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein.

As used herein, the term “specifically binds”, “binding” or simply “binds” or other related terms in the context of the binding of an antigen binding protein to its target antigen means that the antigen binding protein exhibits essentially background binding to non-target molecules. An antigen binding protein that specifically binds the target antigen (e.g., CEACAM5) may, however, cross-react with CEACAM5 proteins from different species. Typically, a CEACAM5 antigen binding protein specifically binds human CEACAM5 when the dissociation constant (K_{D}) is 10⁻⁷ M or less, such as about 10⁻⁸ M or less, such as about 10⁻⁹ M or less, about 10⁻¹⁰ M or less, or about 10⁻¹¹ M or even less as measured via a surface plasma resonance (SPR) technique (e.g., BIACore, GE-Healthcare Uppsala, Sweden) using the antibody as the ligand and the antigen as the analyte.

The term “K_{D}” (M), as used herein, refers to the dissociation equilibrium constant of a particular antigen binding protein-antigen interaction (e.g., antibody-antigen interaction). Affinity, as used herein, and K_{D} are inversely related, such that higher affinity is intended to refer to lower K_{D}, and lower affinity is intended to refer to higher K_{D}.

An “antibody-drug-conjugate” or simply “ADC” refers to an antibody conjugated to a cytotoxic agent such as a Topoisomerase I inhibitor. An antibody-drug-conjugate typically binds to the target antigen (e.g., CEACAM5) on a cell surface followed by internalization of the antibody-drug-conjugate into the cell where the drug is released.

A “cytotoxic effect” refers to the depletion, elimination and/or killing of a target cell.

A “cytotoxic agent” refers to an agent that has a cytotoxic effect on a cell.

A “cytostatic effect” refers to the inhibition of cell proliferation.

A “cytostatic agent” refers to an agent that has a cytostatic effect on a cell, thereby inhibiting the growth of and/or expansion of a specific subset of cells. Cytostatic agents can be conjugated to an antibody or administered in combination with an antibody.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cellular phagocytosis (ADCP); down regulation of cell surface receptors (e.g. B cell receptor); and B cell activation. Such functions can be affected by, for example, binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of the CD33 targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates antibody-dependent cellular cytotoxicity (ADCC) or antibody-dependent cellular phagocytosis (ADCP). ADCC is mediated by CD16⁺ effector cells through the secretion of membrane pore-forming proteins and proteases, while phagocytosis is mediated by CD32⁺ and CD64⁺ effector cells (see, e.g., Fundamental Immunology, 4^{th} ed., Paul ed., Lippincott-Raven, N.Y., 1997, Chapters 3, 17 and 30; Uchida et al., 2004, J. Exp. Med. 199:1659-69; Akewanlop et al., 2001, Cancer Res. 61:4061-65; Watanabe et al., 1999, Breast Cancer Res. Treat. 53:199-207.

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a mechanism of cytotoxicity in which the Fc region of antibodies bound to antigen on the cell surface of target cells interact with Fc receptors (FcRs) present on certain cytotoxic effector cells (e.g. NK cells, neutrophils, and macrophages). This interaction enables these cytotoxic effector cells to subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 or 6,737,056 (Presta), can be performed. Useful effector cells for such assays include PBMC and NK cells. ADCC activity of the molecule of interest can also be assessed in vivo, e.g., in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, e.g., in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to the Fc region of antibodies (of the appropriate subclass), which are bound to their cognate antigen on a target cell. This binding activates a series of enzymatic reactions culminating in the formation of holes in the target cell membrane and subsequent cell death. Activation of complement may also result in deposition of complement components on the target cell surface that facilitate ADCC by binding complement receptors (e.g., CR3) on leukocytes. To assess complement activation, a CDC assay, e.g., as described in Gazzano-Santoro et al., J. Immunol. Methods 202:163 (1996), can be performed. Polypeptide variants with altered Fc region amino acid sequences (polypeptides such as an antibody with a variant Fc region) and increased or decreased C1q binding capability are described, e.g., in U.S. Pat. No. 6,194,551 B1, U.S. Pat. Nos. 7,923,538, 7,994,290 and WO 1999/51642. See also, e.g., Idusogie et al., J. Immunol. 164: 4178-4184 (2000).

The term “antibody-dependent cellular phagocytosis”, or simply “ADCP”, refers to the process by which antibody-coated cells are internalized, either in whole or in part, by phagocytic immune cells (e.g., macrophages, neutrophils and dendritic cells) that bind to an Fc region of Ig.

The terms “nucleic acid molecule”, “nucleic acid” and “polynucleotide” are used interchangeably herein and refer to a polymer of nucleotides of any length. Such polymers of nucleotides can contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The term “vector” means any molecule or entity (e.g., nucleic acid, plasmid, bacteriophage or virus) used to transfer a nucleic acid molecule into a host cell. A vector typically includes a nucleic acid molecule engineered to contain a cloned polynucleotide or polynucleotides encoding a polypeptide or polypeptides of interest that can be propagated in a host cell. Examples of vectors include, but are not limited to, plasmids, viral vectors, and expression vectors, for example, recombinant expression vectors. A vector may include one or more of the following elements: an origin of replication, one or more regulatory sequences (such as, for example, promoters and/or enhancers) that regulate the expression of the polypeptide of interest, and/or one or more selectable marker genes. The term includes vectors which are self-replicating nucleic acid molecules as well as vectors incorporated into the genome of a host cell into which it has been introduced.

The term “expression vector” refers to a vector that is suitable for transformation of a host cell and that can be used to express a polypeptide of interest in a host cell.

The terms “host cell” or “host cell line” are used interchangeably herein and refer to a cell or population of cells that may be or has been a recipient of a vector or isolated polynucleotide. Host cells can be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively. Such terms refer not only to the original cell, but also to the progeny of such a cell. Certain modifications may occur in succeeding generations due to, for example, mutation or environmental influences. Such progeny are also encompassed by the terms so long as the cells have the same function or biological activity as the original cells.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. Control sequences for eukaryotes can include, for example, promoters comprising one or a plurality of recognition sites for transcription factors, transcription enhancer sequences, and transcription termination sequence. “Control sequences” can include leader sequences and/or fusion partner sequences.

As used herein, “operably linked” means that the components to which the term is applied are in a relationship that allows them to carry out their inherent functions under suitable conditions. For example, a control sequence in a vector that is “operably linked” to a protein coding sequence is ligated thereto such that expression of the protein coding sequence is achieved under conditions compatible with the transcriptional activity of the control sequences. In the case in which two encoding sequences are operably linked, the phrase means that the two DNA fragments or encoding sequences are joined such that the amino acid sequences encoded by the two fragments remain in-frame.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual”, “subject”, or patient are used interchangeably herein to refer to an animal, for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some instances, the “individual” or “subject” is a human. In some examples, an “individual” or “subject” refers to an individual or subject (e.g., a human) in need of treatment for a disease or disorder.

A “disease” or “disorder” as used herein refers to a condition where treatment is needed, such as cancer.

“Cancer” and “tumor,” as used herein, are interchangeable terms that refer to any abnormal cell or tissue growth or proliferation in an animal. A solid tumor is an abnormal growth or mass of tissue that usually does not contain cysts or liquid areas. More particular non-limiting examples of such cancers include neuroendocrine cancers, colorectal cancer, stomach cancers, lung cancers, uterus cancers, cervical cancers, pancreatic cancers, esophagus cancers, ovarian cancers, thyroid cancers, bladder cancers, endometrium cancers, bladder cancers, endometrial cancers, breast cancers, liver cancers, prostate cancers, gastric cancers, and cholangiocarcinoma and skin cancers.

The terms “metastatic cancer” and “metastatic disease” mean cancers that have spread from the site of origin to another part of the body, e.g., to regional lymph nodes or to distant sites.

As used herein, “treatment” is an approach for obtaining beneficial or desired clinical results. “Treatment” as used herein, covers any administration or application of a therapeutic for disease in a mammal, including a human. Beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease.

In the context of cancer, the term “treating” includes any or all of: inhibiting growth of cancer cells, inhibiting replication of cancer cells, reducing the number of cancer cells, reducing the rate of cancer cell infiltration into peripheral organs, reducing the rate or extent of tumor metastasis, lessening of overall tumor burden, and ameliorating one or more symptoms associated with the cancer.

A “reference” as used herein, refers to any sample, standard, or level that is used for comparison purposes. A reference can be obtained from a healthy and/or non-diseased sample. In some examples, a reference can be obtained from an untreated sample. In some examples, a reference is obtained from a non-diseased on non-treated sample of a subject individual. In some examples, a reference is obtained from one or more healthy individuals who are not the subject or patient.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

An “effective amount” or “therapeutically effective amount” or “therapeutically effective dosage” of a drug or therapeutic agent is any amount of the drug or agent that, when used alone or in combination with another therapeutic agent provides a treatment effect, such as protecting a subject against the onset of a disease or promoting disease regression as evidenced by a decrease in severity of disease symptoms, an increase in frequency and duration of disease symptom-free periods, or a prevention of impairment or disability due to the disease affliction.

“Administering” or “administration” refer to the physical introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods.

The term “chemotherapeutic agent” refers to all chemical compounds that are effective in inhibiting tumor growth. Non-limiting examples of chemotherapeutic agents include alkylating agents (e.g., nitrogen mustards, ethyleneimine compounds and alkyl sulphonates); antimetabolites (e.g., folic acid, purine or pyrimidine antagonists); mitotic inhibitors (e.g., anti-tubulin agents such as vinca alkaloids, auristatins and derivatives of podophyllotoxin); cytotoxic antibiotics; compounds that damage or interfere with DNA expression or replication (e.g., DNA minor groove binders); and growth factor receptor antagonists, and cytotoxic or cytostatic agents.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Compound” as the term is used herein, refers to and encompasses the chemical compound itself, either named or represented by structure, and salt form(s) thereof, whether explicitly stated or not, unless context makes clear that such salt forms are to be excluded. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; and when a compound can be associated with two or more solvent molecules, the two or more solvent molecules may be the same or different.

In some instances, a compound of the invention will include an explicit reference to one or more of the above forms, e.g., salts and solvates, which does not imply any solid state form of the compound; however, this reference is for emphasis only, and is not to be construed as excluding any other of the forms as identified above. Furthermore, when explicit reference to a salt and/or solvate form of a compound or a Ligand Drug Conjugate composition is not made, that omission is not to be construed as excluding the salt and/or solvate form(s) of the compound or Conjugate unless context make clear that such salt and/or solvate forms are to be excluded.

A pharmaceutically acceptable salt is a salt form of a compound that is suitable for administration to a subject as described herein and in some aspects includes countercations or counteranions as described by P. H. Stahl and C. G. Wermuth, editors, Handbook of Pharmaceutical Salts: Properties, Selection and Use, Weinheim/Zurich:Wiley-VCH/VHCA, 2002.

A Linker Unit is a bifunctional moiety that connects a Camptothecin to a Ligand Unit in an ADC. The Linker Units of the present invention have several components (e.g., a Stretcher Unit which in some embodiments will have a Basic Unit; a Connector Unit, that can be present or absent; a Parallel Connector Unit, that can also be present or absent; a Releasable Linker; and a Spacer Unit, that can also be present or absent).

“PEG”, “PEG Unit” or “polyethylene glycol” as used herein is an organic moiety comprised of repeating ethylene-oxy subunits and may be polydisperse, monodisperse or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein comprises one or multiple polyethylene glycol chains, each comprised of one or more ethyleneoxy subunits, covalently attached to each other. The polyethylene glycol chains can be linked together, for example, in a linear, branched or star shaped configuration. Typically, at least one of the polyethylene glycol chains prior to incorporation into an ADC is derivitized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically an optionally substituted alkyl such as —CH₃, —CH₂CH₃ or —CH₂CH₂CO₂H. A preferred PEG Unit has a single polyethylene glycol chain with 4 to 24 —CH₂CH₂O— subunits covalently attached in series and terminated at one end with a PEG Capping Unit.

Unless otherwise indicated, the term “alkyl” by itself or as part of another term refers to a substituted or unsubstituted straight chain or branched, saturated or unsaturated hydrocarbon having the indicated number of carbon atoms (e.g., “—C₁-C₈ alkyl” or”—C₁-C₁₀ alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl, -n-propyl, -n-butyl, -n-pentyl, -n-hexyl, -n-heptyl and -n-octyl; while branched —C₃-C₈ alkyls include, but are not limited to, -isopropyl, -sec-butyl, -isobutyl, -tert-butyl, -isopentyl, and -2-methylbutyl; unsaturated —C₂-C₈ alkyls include, but are not limited to, -vinyl, -allyl, -1-butenyl, -2-butenyl, -isobutylenyl, -1 pentenyl, -2 pentenyl, -3-methyl-1-butenyl, -2 methyl-2-butenyl, -2,3 dimethyl-2-butenyl, -1-hexyl, 2-hexyl, -3-hexyl, -acetylenyl, -propynyl, -1 butynyl-2 butynyl, -1 pentynyl, -2 pentynyl and -3 methyl 1 butynyl. Sometimes an alkyl group is unsubstituted. An alkyl group can be substituted with one or more groups. In other aspects, an alkyl group will be saturated.

Unless otherwise indicated, “alkylene,” by itself of as part of another term, refers to a substituted or unsubstituted saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon).

Unless otherwise indicated, “aryl,” by itself or as part of another term, means a substituted or unsubstituted monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene,” by itself or as part of another term, is an aryl group as defined above which has two covalent bonds (i.e., it is divalent) and can be in the ortho, meta, or para orientations as shown in the following structures, with phenyl as the exemplary group:

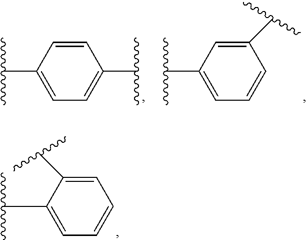

Unless otherwise indicated, a “C₃-C₈ heterocycle,” by itself or as part of another term, refers to a monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Heterocycles in which all the ring atoms are involved in aromaticity are referred to as heteroaryls and otherwise are referred to heterocarbocycles.

Unless otherwise noted, the heterocycle is attached to its pendant group at any heteroatom or carbon atom that results in a stable structure. As such a heteroaryl may be bonded through an aromatic carbon of its aromatic ring system, referred to as a C-linked heteroaryl, or through a non-double-bonded N atom (i.e., not ═N—) in its aromatic ring system, which is referred to as an N-linked heteroaryl. Thus, nitrogen-containing heterocycles may be C-linked or N-linked and include pyrrole moieties, such as pyrrol-1-yl (N-linked) and pyrrol-3-yl (C-linked), and imidazole moieties such as imidazol-1-yl and imidazol-3-yl (both N-linked), and imidazol-2-yl, imidazol-4-yl and imidazol-5-yl moieties (all of which are C-linked).

Unless otherwise indicated, a “C₃-C₈ heteroaryl,” is an aromatic C₃-C₈ heterocycle in which the subscript denotes the total number of carbons of the cyclic ring system of the heterocycle or the total number of aromatic carbons of the aromatic ring system of the heteroaryl and does not implicate the size of the ring system or the presence or absence of ring fusion. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

When explicitly given, the size of the ring system of a heterocycle or heteroaryl is indicated by the total number of atoms in the ring. For example, designation as a 5- or 6-membered heteroaryl indicates the total number or aromatic atoms (i.e., 5 or 6) in the heteroaromatic ring system of the heteroaryl but does not imply the number of aromatic heteroatoms or aromatic carbons in that ring system. Fused heteroaryls are explicitly stated or implied by context as such and are typically indicated by the number of aromatic atoms in each aromatic ring that are fused together to make up the fused heteroaromatic ring system. For example, a 5,6-membered heteroaryl is an aromatic 5-membered ring fused to an aromatic 6-membered ring in which one or both rings have aromatic heteroatom(s) or where a heteroatom is shared between the two rings.

A heterocycle fused to an aryl or heteroaryl such that the heterocycle remains non-aromatic and is part of a larger structure through attachment with the non-aromatic portion of the fused ring system is an example of an optionally substituted heterocycle in which the heterocycle is substituted by ring fusion with the aryl or heteroaryl. Likewise, an aryl or heteroaryl fused to heterocycle or carbocycle that is part of a larger structure through attachment with the aromatic portion of the fused ring system is an example of an optionally substituted aryl or heterocycle in which the aryl or heterocycle is substituted by ring fusion with the heterocycle or carbocycle.

Unless otherwise indicated, “C₃-C₈ heterocyclo,” by itself or as part of another term, refers to a C₃-C₈ heterocyclic defined above wherein one of the hydrogen atoms of the heterocycle is replaced with a bond (i.e., it is divalent). Unless otherwise indicated, a “C₃-C₈ heteroarylene,” by itself or as part of another term, refers to a C₃-C₈ heteroaryl group defined above wherein one of the heteroaryl group's hydrogen atoms is replaced with a bond (i.e., it is divalent).

Unless otherwise indicated, a “C₃-C₈ carbocycle,” by itself or as part of another term, is a 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo,” by itself or as part of another term, refers to a C₃-C₈ carbocycle group defined above wherein another one of the carbocycle groups' hydrogen atoms is replaced with a bond (i.e., it is divalent).

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si can be placed at any position of the heteroalkyl group, including the position at which the alkyl group is attached to the remainder of the molecule.

Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CHO—CH₃, —Si(CH₃)₃, —CH₂—CH═NO—CH₃, and —CH═CH—N(CH₃)—CH₃. Up to two heteroatoms may be consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

Unless otherwise indicated, “aminoalkyl” by itself or in combination with another term means a heteroalkyl wherein an alkyl moiety as defined herein is substituted with an amino, alkylamino, dialkylamino or cycloalkylamino group. Exemplary non-limiting aminoalkyls are —CH₂NH₂, —CH₂CH₂NH₂, —CH₂CH₂NHCH₃ and —CH₂CH₂N(CH₃)₂ and further includes branched species such as —CH(CH₃)NH₂ and —C(CH₃)CH₂NH₂ in the (R)- or (S)-configuration. Alternatively, an aminoalkyl is an alkyl moiety, group, or substituent as defined herein wherein a sp³ carbon other than the radical carbon has been replaced with an amino or alkylamino moiety wherein its sp³ nitrogen replaces the sp³ carbon of the alkyl provided that at least one sp³ carbon remains. When referring to an aminoalkyl moiety as a substituent to a larger structure or another moiety the aminoalkyl is covalently attached to the structure or moiety through the carbon radical of the alkyl moiety of the aminoalkyl.

Unless otherwise indicated “alkylamino” and “cycloalkylamino” by itself or in combination with another term means an alkyl or cycloalkyl radical, as described herein, wherein the radical carbon of the alkyl or cycloalkyl radical has been replaced with a nitrogen radical, provided that at least one sp³ carbon remains. In those instances where the alkylamino is substituted at its nitrogen with another alkyl moiety the resulting substituted radical is sometimes referred to as a dialkylamino moiety, group or substituent wherein the alkyl moieties substituting nitrogen are independently selected.

Exemplary and non-limiting amino, alkylamino and dialkylamino substituents, include those having the structure of —N(R′)₂, wherein R′ in these examples are independently selected from hydrogen or C₁₋₆ alkyl, typically hydrogen or methyl, whereas in cycloalkyl amines, which are included in heterocycloalkyls, both R′ together with the nitrogen to which they are attached define a heterocyclic ring. When both R′ are hydrogen or alkyl, the moiety is sometimes described as a primary amino group and a tertiary amine group, respectively. When one R′ is hydrogen and the other is alkyl, then the moiety is sometimes described as a secondary amino group. Primary and secondary alkylamino moieties are more reactive as nucleophiles towards carbonyl-containing electrophilic centers whereas tertiary amines are more basic.

“Substituted alkyl” and “substituted aryl” mean alkyl and aryl, respectively, in which one or more hydrogen atoms, typically one, are each independently replaced with a substituent. Typical substituents include, but are not limited to a —X, —R′, —OH, —OR′, —SR′, —N(R′)₂, —N(R′)₃, ═NR′, —CX₃, —CN, —NO₂, —NR′C(═O)R′, —C(═O)R′, —C(═O)N(R′)₂, —S(═O)₂R′, —S(═O)₂NR′, —S(═O)R′, —OP(═O)(OR′)₂, —P(═O)(OR′)₂, —PO₃—, PO₃H₂, —C(═O)R′, —C(═S)R′, —CO₂R′, —CO₂—, —C(═S)OR, —C(═O)SR′, —C(═S)SR′, —C(═O)N(R′)₂, —C(═S)N(R′)₂, and —C(═NR)N(R′)₂, where each X is independently selected from the group consisting of a halogen: —F, —Cl, —Br, and —I; and wherein each R′ is independently selected from the group consisting of —H, —C₁-C₂₀ alkyl, —C₆-C₂₀ aryl, —C₃-C₁₄ heterocycle, a protecting group, and a prodrug moiety.

More typically substituents are selected from the group consisting of —X, —R′, —OH, —OR′, —SR′, —N(R′)₂, —N(R′)₃, ═NR′, —NR′C(═O)R′, —C(═O)R′, —C(═O)N(R′)₂, —S(═O)₂R′, —S(═O)₂NR′, —S(═O)R′, —C(═O)R′, —C(═S)R′, —C(═O)N(R′)₂, —C(═S)N(R′)₂, and —C(═NR)N(R′)₂, wherein each X is independently selected from the group consisting of —F and —Cl, or are selected from the group consisting of —X, —R′, —OH, —OR′, —N(R′)₂, —N(R′)₃, —NR′C(═O)R′, —C(═O)N(R′)₂, —S(═O)₂R′, —S(═O)₂NR′, —S(═O)R′, —C(═O)R′, —C(═O)N(R′)₂, —C(═NR)N(R′)₂, a protecting group, and a prodrug moiety, wherein each X is —F; and wherein each R′ is independently selected from the group consisting of hydrogen, —C₁-C₂₀ alkyl, —C₆-C₂₀ aryl, —C₃-C₁₄ heterocycle, a protecting group, and a prodrug moiety.

In some aspects, an alkyl substituent is selected from the group consisting —N(R′)₂, —N(R′)₃ and —C(═NR)N(R′)₂, wherein R′ is selected from the group consisting of hydrogen and —C₁-C₂₀ alkyl. In other aspects, alkyl is substituted with a series of ethyleneoxy moieties to define a PEG Unit. Alkylene, carbocycle, carbocyclo, arylene, heteroalkyl, heteroalkylene, heterocycle, heterocyclo, heteroaryl, and heteroarylene groups as described above may also be similarly substituted.

“Protecting group” as used here means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, Wiley Interscience. Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by —ORPR, wherein RPR is a protecting group for hydroxyl, wherein hydroxyl is typically protected as an ester (e.g. acetate, propionate or benzoate). Other protecting groups for hydroxyl avoid interfering with the nucleophilicity of organometallic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. By way of example and not limitation, a suitable protecting group may include those previously described for protecting functional groups. A suitable protecting group is sometimes a protecting group used in peptide coupling reactions.

“Aromatic alcohol” by itself or part of a larger structure refers to an aromatic ring system substituted with the hydroxyl functional group —OH. Thus, aromatic alcohol refers to any aryl, heteroaryl, arylene and heteroarylene moiety as described herein having a hydroxyl functional group bonded to an aromatic carbon of its aromatic ring system. The aromatic alcohol may be part of a larger moiety as when its aromatic ring system is a substituent of this moiety, or may be embeded into the larger moiety by ring fusion, and may be optionally substituted with moieties as described herein including one or more other hydroxyl substitutents. A phenolic alcohol is an aromatic alcohol having a phenol group as the aromatic ring.

“Aliphatic alcohol” by itself or part of a larger structure refers to a moiety having a non-aromatic carbon bonded to the hydroxyl functional group —OH. The hydroxy-bearing carbon may be unsubstituted (i.e., methyl alcohol) or may have one, two or three optionally substitued branched or unbranched alkyl substituents to define a primary alcohol, or a secondary or tertiary aliphatic alcohol wihin a linear or cyclic structure. When part of a larger structure, the alcohol may be a substituent of this structure by bonding through the hydroxy bearing carbon, through a carbon of an alkyl or other moiety as described herein to this hydroxyl-bearing carbon or through a substituent of this alkyl or other moiety. An aliphatic alchohol contemplates a non-aromatic cyclic structure (i.e., carbocycles and heterocarbocycles, optionally substituted) in which a hydroxy functional group is bonded to a non-aromatic carbon of its cyclic ring system.

“Arylalkyl” or “heteroarylalkyl” as used herein means a substituent, moiety or group where an aryl moiety is bonded to an alkyl moiety, i.e., aryl-alkyl-, where alkyl and aryl groups are as described above, e.g., C₆H₅—CH₂— or C₆H₅—CH(CH₃)CH₂—. An arylalkyl or heteroarylalkyl is associated with a larger structure or moiety through a sp³ carbon of its alkyl moiety.

“Electron withdrawing group” as used herein means a functional group or electronegative atom that draws electron density away from an atom to which it is bonded either inductively and/or through resonance, whichever is more dominant (i.e., a functional group or atom may be electron withdrawing inductively but may overall be electron donating through resonance) and tends to stabilize anions or electron-rich moieties. The electron withdrawing effect is typically transmitted inductively, albeit in attenuated form, to other atoms attached to the bonded atom that has been made electron deficient by the electron withdrawing group (EWG), thus affecting the electrophilicity of a more remote reactive center. Exemplary electron withdrawing groups include, but are not limited to —C(═O), —CN, —NO₂, —CX₃, —X, —C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃*, wherein X is —F, —Br, —Cl, or —I, and R′ in some aspects is, at each occurrence, independently selected from the group consisting of hydrogen and C₁₋₆ alkyl, and certain O-linked moieties as described herein such as acyloxy.

Exemplary EWGs can also include aryl groups (e.g., phenyl) depending on substitution and certain heteroaryl groups (e.g., pyridine). Thus, the term “electron withdrawing groups” also includes aryls or heteroaryls that are further substituted with electron withdrawing groups. Typically, electron withdrawing groups on aryls or heteroaryls are —C(═O), —CN, —NO₂, —CX₃, and —X, wherein X independently selected is halogen, typically —F or —Cl. Depending on their substituents, an alkyl moiety may also be an electron withdrawing group.

“Electron-donating group”, as the term is used herein, unless otherwise stated or implied by context, refers to a functional group or electropositive atom that increases electron density of an atom to which it is bonded either inductively and/or through resonance, whichever is more dominant (i.e., a functional group or atom may be electron-withdrawing inductively but may overall be electron-donating through resonance), and tends to stabilize cations or electron poor systems. The electron-donating effect is typically transmitted through resonance to other atoms attached to the bonded atom that has been made electron rich by the electron-donating group (EDG) thus increasing the electron density of a more remote reactive center. Typically, an electron donating group is selected from the group consisting of —OH, —OR′, —NH₂, —NHR′, and N(R′)₂, wherein each R′ is an independently selected from C₁-C₁₂ alkyl, typically C₁-C₆ alkyl. Depending on its substituents, a C₆-C₂₄ aryl, C₅-C₂₄ heteroaryl, or unsaturated C₁-C₁₂ alkyl moiety may also be an electron-donating group, and in some aspects, such moieties are encompassed by the term for an electron-donating group.

“Leaving group ability” relates to the ability of an alcohol-, thiol-, amine- or amide-containing compound corresponding to a Camptothecin in an ADC to be released from the Conjugate as a free drug subsequent to activation of a self-immolative event within the Conjugate. That release can be variable without the benefit of a methylene carbamate unit to which its Camptothecin is attached (i.e., when the Camptothecin is directly attached to a self-immolative moiety and does not have an intervening methylene carbamate unit). Good leaving groups are usually weak bases and the more acidic the functional group that is expelled from such conjugates the weaker the conjugate base is. Thus, the leaving group ability of an alcohol-, thiol-, amine- or amide-containing free drug from a Camptothecin will be related to the pKa of the drug's functional group that is expelled from a conjugate in cases where methylene carbamate unit (i.e., one in which a Camptothecin is directly attached to a self-immolative moiety) is not used. Thus, a lower pKa for that functional group will increase its leaving group ability. Although other factors may contribute to release of free drug from conjugates not having the benefit of a methylene carbamate unit, generally a drug having a functional group with a lower pKa value will typically be a better leaving group tha a drug attached via a functional group with a higher pKa value. Another consideration is that, a functional group having too low of a pKa value may result in an unacceptable activity profile due to premature loss of the Camptothecin via spontaneous hydrolysis. For conjugates employing a methylene carbamate unit, a common functional group (i.e., a carbamic acid) having a pKa value that allows for efficient release of free drug, without suffering unacceptable loss of Camptothecin, is produced upon self-immolation.

“Succinimide moiety” as used herein refers to an organic moiety comprised of a succinimide ring system, which is present in one type of Stretcher Unit (Z) that is typically further comprised of an alkylene-containing moiety bonded to the imide nitrogen of that ring system. A succinimide moiety typically results from Michael addition of a sulfhydryl group of a Ligand Unit to the maleimide ring system of a Stretcher Unit precursor (Z′). A succinimide moiety is therefore comprised of a thio-substituted succinimide ring system and when present in an ADC has its imide nitrogen substituted with the remainder of the Linker Unit of the ADC and is optionally substituted with substituent(s) that were present on the maleimide ring system of Z′.

“Acid-amide moiety” as used herein refers to succinic acid having an amide substituent that results from the thio-substituted succinimide ring system of a succinimide moiety having undergone breakage of one of its carbonyl-nitrogen bonds by hydrolysis. Hydrolysis resulting in a succinic acid-amide moiety provides a Linker Unit less likely to suffer premature loss of the Ligand Unit to which it is bonded through elimination of the antibody-thio substituent. Hydrolysis of the succinimide ring system of the thio-substituted succinimide moiety is expected to provide regiochemical isomers of acid-amide moieties that are due to differences in reactivity of the two carbonyl carbons of the succinimide ring system attributable at least in part to any substituent present in the maleimide ring system of the Stretcher Unit precursor and to the thio substituent introduced by the targeting ligand.

The term “Prodrug” as used herein refers to a less biologically active or inactive compound which is transformed within the body into a more biologically active compound via a chemical or biological process (i.e., a chemical reaction or an enzymatic biotransformation). Typically, a biologically active compound is rendered less biologically active (i.e., is converted to a prodrug) by chemically modifying the compound with a prodrug moiety. In some aspects, the prodrug is a Type II prodrug, which are bioactivated outside cells, e.g., in digestive fluids, or in the body's circulation system, e.g., in blood. Exemplary prodrugs are esters and β-D-glucopyranosides.

In many instances, the assembly of the conjugates, linkers and components described herein will refer to reactive groups. A “reactive group” or RG is a group that contains a reactive site (RS) capable of forming a bond with either the components of the Linker unit (i.e., A, W, Y) or the Camptothecin D. RS is the reactive site within a Reactive Group (RG). Reactive groups include sulfhydryl groups to form disulfide bonds or thioether bonds, aldehyde, ketone, or hydrazine groups to form hydrazone bonds, carboxylic or amino groups to form peptide bonds, carboxylic or hydroxy groups to form ester bonds, sulfonic acids to form sulfonamide bonds, alcohols to form carbamate bonds, and amines to form sulfonamide bonds or carbamate bonds.

The following table is illustrative of Reactive Groups, Reactive Sites, and exemplary functional groups that can form after reaction of the reactive site. The table is not limiting. One of skill in the art will appreciate that the noted R′ and R″ portions in the table are effectively any organic moiety (e.g., an alkyl group, aryl group, heteroaryl group, or substituted alkyl, aryl, or heteroaryl, group) which is compatible with the bond formation provided in converting RG to one of the Exemplary Functional Groups. It will also be appreciated that, as applied to the embodiments of the present invention, R′ may represent one or more components of the self-stabilizing linker or optional secondary linker, as the case may be, and R″ may represent one or more components of the optional secondary linker, Camptothecin, stabilizing unit, or detection unit, as the case may be.

A “sterile” formulation is aseptic or essentially free from living microorganisms and their spores.

Various aspects of the disclosure are described in further detail in the following sections.

### II. CEACAM5

Domain organization of human CEACAM5 is as follows (based on GenBank AAA51967.1 sequence; SEQ ID NO:11):

| Human CEACAM5 domains | Positions on SEQ ID NO: 11 |
|---|---|
| Domain N | 35-142 |
| Domain A1 | 143-237 |
| Domain B1 | 238-320 |
| Domain A2 | 321-415 |
| Domain B2 | 416-498 |
| Domain A3 | 499-593 |
| Domain B3 | 594-685 |

Accordingly, the A3-B3 domain of human CEACAM5 consists of amino acids at positions 499-685 of SEQ ID NO:11.

Domain organisation of Macaca fascicularis CEACAM5 is as follows (based on cloned extracellular domain sequence; SEQ ID NO:12):

TABLE 2

| Macaca fascicularis CEACAM5 domains | Positions on SEQ ID NO: 12 |
|---|---|
| Domain N-A1-B1 | −1-286 |
| Domain A2-B2 | −287-464 |
| Domain A3-B3 | 465-654 |

### III. Anti-CEACAM5 ADCs

In some embodiments, provided herein are ADCs having a formula:

L-(Q-D)ₚ

or a salt thereof, wherein
L is a Ligand Unit comprising an antibody or antigen binding fragment thereof that binds to CEACAM5;
the subscript p is an integer of from 1 to 16; Q is a Linker Unit;
and D is a Drug Unit, wherein the Drug Unit is a Topoisomerase I inhibitor.
In some embodiments, the ADC provided herein, have certain advantages over other ADCs, including increased anti-tumor activity and decreased toxicity.

### A. CEACAM5 Antibodies

According to an embodiment, the antibody according to the invention is specific for the surface human and Macaca fascicularis CEACAM5 proteins. In an embodiment, the antibody of the invention does not bind to, or does not significantly cross-react with human CEACAM1, human CEACAM6, human CEACAM7, human CEACAM8, Macaca fascicularis CEACAM1, Macaca fascicularis CEACAM6 and Macaca fascicularis CEACAM8 proteins.

In particular, the antibody does not bind to, or does not significantly cross-react with the extracellular domain of the aforementioned human and Macaca fascicularis CEACAM proteins.

In particular, the antibody binds to the A3-B3 domain of CEACAM5.

An embodiment of the invention has an affinity for human CEACAM5 or Macaca fascicularis CEACAM5, or both, which is ≤10 nM, for instance ≤5 nM, ≤3 nM, ≤1 nM or ≤0.1 nM, for instance an affinity of 0.01 nM to 5 nM, or and affinity of 0.1 nM to 5 nM, or of 0.1 nM to 1 nM.

Affinity for human CEACAM5 or for Macaca fascicularis CEACAM5 may be determined as the EC50 value in an ELISA using soluble recombinant CEACAM5 as capture antigen. The antibody of the invention may also have an apparent dissociation constant (apparent KD), as may be determined by FACS analysis on tumor cell line MKN45 (DSMZ, ACC 409) or on xenograft tumor cells deriving from patient (CR-IGR-034P) available from Oncodesign Biotechnology, tumor collection CReMEC), which is ≤25 nM, for instance ≤20 nM, ≤10 nM, ≤5 nM, ≤3 nM or ≤1 nM. The apparent KD may be within the range 0.01-20 nM, or may be within the range 0.1-20 nM, 0.1-10 nM, or 0.1-5 nM. Additionally, antibodies according to the invention have been shown to be able to detect CEACAM5 expression by immunohistochemistry in frozen and formalin-fixed and paraffin embedded (FFPE) tissue sections.

In some embodiments, the anti-CEACAM5 antibody comprises a Fc region. In some embodiments, the anti-CEACAM5 antibody is a Fc-competent antibody. In some embodiments, a Fc competent antibody may trigger an ADCC and/or ADCP activity, improving the activity.

In some embodiments, the anti-CEACAM5 antibody comprises one or more of a CDR1-H comprising the amino acid sequence set forth in SEQ ID NO:1; a CDR2-H comprising the amino acid sequence set forth in SEQ ID NO:2; a CDR3-H comprising the amino acid sequence set forth in SEQ ID NO:3; a CDR1-L comprising the amino acid sequence set forth in SEQ ID NO:4; a CDR2-L comprising the amino acid sequence NTR; and a CDR3-L comprising the amino acid sequence set forth in SEQ ID NO:6.

In some embodiments, the anti-CEACAM5 antibody comprises one or more of a CDR1-H comprising the amino acid sequence set forth in SEQ ID NO:1; a CDR2-H comprising the amino acid sequence set forth in SEQ ID NO:2; a CDR3-H comprising the amino acid sequence set forth in SEQ ID NO:3; a CDR1-L comprising the amino acid sequence set forth in SEQ ID NO:4; a CDR2-L comprising the amino acid sequence NTR; and a CDR3-L comprising the amino acid sequence set forth in SEQ ID NO:6.

In some embodiments, the antibody or antigen binding fragment thereof comprises a VH that has at least 80%, 85%, 90%, 95% or 99% amino acid sequence identity to the amino acid sequence set forth in SEQ ID NO:7. In some embodiments, the antibody or antigen binding fragment thereof comprises a VL that has at least 80%, 85%, 90%, 95% or 99% amino acid sequence identity to the amino acid sequence set forth in SEQ ID NO:8. In some embodiments, the antibody or antigen binding fragment thereof comprises a VH that has at least 80%, 85%, 90%, 95% or 99% amino acid sequence identity to the amino acid sequence set forth in SEQ ID NO:7 and a VL that has at least 80%, 85%, 90%, 95% or 99% amino acid sequence identity to the amino acid sequence set forth in SEQ ID NO:8.

In some embodiments, the antigen binding protein comprises a CDR1-H, a CDR2-H, and a CDR3-H of a variable heavy chain domain (VH) comprising the amino acid sequence set forth in SEQ ID NO:7. In some embodiments, the antibody or antigen binding fragment thereof comprises a CDR1-L, a CDR2-L, and a CDR3-L of a variable light chain domain (VL) comprising the amino acid sequence set forth in SEQ ID NO:8. In some embodiments, the antigen binding protein comprises a CDR1-H, a CDR2-H, and a CDR3-H of a variable heavy chain domain (VH) comprising the amino acid sequence set forth in SEQ ID NO:7 and a CDR1-L, a CDR2-L, and a CDR3-L of a variable light chain domain (VL) comprising the amino acid sequence set forth in SEQ ID NO:8.

In some embodiments, the anti-CEACAM5 antibody comprises one or more of a CDR1-H comprising the amino acid sequence set forth in SEQ ID NO:1; a CDR2-H comprising the amino acid sequence set forth in SEQ ID NO: 2; a CDR3-H comprising the amino acid sequence set forth in SEQ ID NO:3 and a VH comprising at least 80%, 85%, 90%, 95% or 99% amino acid sequence identity with the amino acid sequence set forth in SEQ ID NO:7. In some embodiments, the antibody or antigen binding fragment thereof comprises a CDR1-L comprising the amino acid sequence set forth in SEQ ID NO:4; a CDR2-L comprising the amino acid sequence NTR; and a CDR3-L comprising the amino acid sequence set forth in SEQ ID NO:6 and a VL comprising at least 80%, 85%, 90%, 95% or 99% amino acid sequence identity with the amino acid sequence set forth in SEQ ID NO:8.

In other embodiments, the antibody or antigen binding fragment thereof comprises a VL comprising a CDR1-L, an CDR2-L, and an CDR3-L, wherein the CDRs of the VL collectively have at most 1, 2, 3, 4, or 5 amino acid changes relative to a corresponding CDR reference sequence, and wherein the CDR1-L reference sequence has the amino acid sequence of SEQ ID NO:4, the CDR2-L has the amino acid sequence set forth in SEQ ID NO:5 and the CDR-3L has the amino acid sequence set forth in SEQ ID NO:6. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In some embodiments, the antibody or antigen binding fragment thereof comprises a VH comprising a CDR1-H, a CDR2-H, and a CDR3-H, wherein the CDRs of the VH collectively have at most 1, 2, 3, 4, or 5 amino acid changes relative to a corresponding CDR reference sequence, and wherein the CDR1-H reference sequence has the amino acid sequence of SEQ ID NO:1, the CDR2-H has the amino acid sequence set forth in SEQ ID NO:2 and the CDR3-H has the amino acid sequence set forth in SEQ ID NO:3. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In some embodiments, the antibody or antigen binding fragment thereof comprises a VH comprising a CDR1-H, a CDR2-H, and a CDR3-H, wherein the CDRs of the VH collectively have at most 1, 2, 3, 4, or 5 amino acid changes relative to a corresponding CDR reference sequence, and wherein the CDR1-H reference sequence has the amino acid sequence of SEQ ID NO:1, the CDR2-H has the amino acid sequence set forth in SEQ ID NO:2 and the CDR3-H has the amino acid sequence set forth in SEQ ID NO:3 and a VL comprising a CDR1-L, an CDR2-L, and an CDR3-L, wherein the CDRs of the VL collectively have at most 1, 2, 3, 4, or 5 amino acid changes relative to a corresponding CDR reference sequence, and wherein the CDR1-L reference sequence has the amino acid sequence of SEQ ID NO:4, the CDR2-L has the amino acid sequence set forth in SEQ ID NO:5 and the CDR-3L has the amino acid sequence set forth in SEQ ID NO:6. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In some embodiments, the antibody or antigen binding fragment thereof comprises a heavy chain that has at least 80%, 85%, 90%, 95% or 99% amino acid sequence identity to the amino acid sequence set forth in SEQ ID NO:9. In some embodiments, the antibody or antigen binding fragment thereof comprises a light chain that has at least 80%, 85%, 90%, 95% or 99% amino acid sequence identity to the amino acid sequence set forth in SEQ ID NO:10. In some embodiments, the antibody or antigen binding fragment thereof comprises a heavy chain that has at least 80%, 85%, 90%, 95% or 99% amino acid sequence identity to the amino acid sequence set forth in SEQ ID NO:9 and a light chain that has at least 80%, 85%, 90%, 95% or 99% amino acid sequence identity to the amino acid sequence set forth in SEQ ID NO:10.

In some embodiments, the antibody or antigen binding fragment thereof comprises a heavy chain comprising the amino acid sequence set forth in SEQ ID NO:9. In some embodiments, the antibody or antigen binding fragment thereof comprises a light chain comprising the amino acid sequence set forth in SEQ ID NO:10. In some embodiments, the antibody or antigen binding fragment thereof comprises a heavy chain comprising the amino acid sequence set forth in SEQ ID NO:9 and a light chain comprising the amino acid sequence set forth in SEQ ID NO:10.

In some embodiments, the antibody or antigen binding fragment thereof comprises a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NO:7, provided the antibody or antigen binding fragment thereof retains the ability to bind to CEACAM5. In certain embodiments, such an antibody or antigen binding fragment thereof contains substitutions (e.g., conservative substitutions), insertions, and/or deletions relative to the reference sequence (i.e., one of SEQ ID NO:7), provided that such an antibody or antigen binding fragment thereof retains the ability to bind to CEACAM5. In certain embodiments, 1, 2, 3, 4, 5, 6, 7, 8, 9, or 10 amino acids have been substituted, inserted and/or deleted in any one of SEQ ID NO:7. In some embodiments, 1-5 or 1-3 amino acids have been substituted, inserted and/or deleted in the VH sequence. In certain of these embodiments, such substitutions, insertions, or deletions occur in regions outside the CDRs (i.e., in the FRs). In further such embodiments, the VH comprises one, two or three CDRs selected from: (a) a CDR1-H comprising the amino acid sequence of SEQ ID NO:1; (b) an CDR2-H comprising the amino acid sequence of SEQ ID NO:2 (c) an CDR3-H comprising the amino acid sequence of SEQ ID NO:3.

In some embodiments, the antibody or antigen binding fragment thereof comprises a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NO:8, provided the antibody or antigen binding fragment thereof retains the ability to bind to CEACAM5. In certain embodiments, such an antibody or antigen binding fragment thereof contains substitutions (e.g., conservative substitutions), insertions, and/or deletions relative to the reference sequence (i.e., one of SEQ ID NO:8), provided that such an antibody or antigen binding fragment thereof retains the ability to bind to CEACAM5. In certain embodiments, 1, 2, 3, 4, 5, 6, 7, 8, 9, or 10 amino acids have been substituted, inserted and/or deleted in any one of SEQ ID NO:8. In some embodiments, 1-5 or 1-3 amino acids have been substituted, inserted and/or deleted in the VL sequence. In certain of these embodiments, such substitutions, insertions, or deletions occur in regions outside the CDRs (i.e., in the FRs). In further such embodiments, the VL comprises one, two or three CDRs selected from: (a) a CDR1-L comprising the amino acid sequence of SEQ ID NO:4; (b) an CDR2-L comprising the amino acid sequence of SEQ ID NO:5 (c) an CDR3-L comprising the amino acid sequence of SEQ ID NO:6.

In a further embodiment, the antibody or antigen binding fragment thereof comprises (a) a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NO:7, and (b) a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NO:8, provided the antibody or antigen binding fragment thereof retains the ability to bind to CEACAM5. In certain embodiments, such an antibody or antigen binding fragment thereof contains substitutions (e.g., conservative substitutions), insertions, and/or deletions relative to the reference sequence (i.e., SEQ ID NO:7 for the VH domain and SEQ ID NO:8 for the VL domain), provided that such an antibody or antigen binding fragment thereof retains the ability to bind to CEACAM5. In certain embodiments, 1, 2, 3, 4, 5, 6, 7, 8, 9, or 10 amino acids have been substituted, inserted and/or deleted in the VH and/or the VL sequence. In some embodiments, 1-5 or 1-3 amino acids have been substituted, inserted and/or deleted in the VH and/or VL sequence. In other embodiments, 1-5 or 1-3 amino acids have been substituted, inserted and/or deleted in the VH and VL sequence collectively. In certain of these embodiments, such substitutions, insertions, or deletions occur in regions outside the HVRs (i.e., in the FRs). In further such embodiments, the VL comprises one, two or three CDRs selected from: (a) a CDR1-L comprising the amino acid sequence of SEQ ID NO:4; (b) an CDR2-L comprising the amino acid sequence of SEQ ID NO:5 (c) an CDR3-L comprising the amino acid sequence of SEQ ID NO:6 and the VH comprises one, two or three CDRs selected from: (a) a CDR1-H comprising the amino acid sequence of SEQ ID NO:1; (b) an CDR2-H comprising the amino acid sequence of SEQ ID NO:2 (c) an CDR3-H comprising the amino acid sequence of SEQ ID NO:3.

The antigen binding protein in any of the foregoing embodiments can be an antibody in any form. As such, the antigen binding protein described in any of the above embodiments can be, for example, a monoclonal antibody, a multispecific antibody, a human, humanized or chimeric antibody, and antigen binding fragments of any of the above, such as a single chain antibody, an Fab fragment, an F(ab′) fragment, or a fragment produced by a Fab expression library. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain embodiments, an antibody or antigen binding fragment thereof with the HVR and/or variable domain sequences described herein is an antigen-binding fragment (e.g., human antigen-binding fragments) and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains.

The antibody or antigen binding fragment thereof can be monospecific, bispecific, trispecific or of greater multi specificity. Multispecific antibodies can be specific for different epitopes of CEACAM5 or may be specific for both CEACAM5 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; and Kostelny et al., 1992, J. Immunol. 148:1547 1553.

In any of the embodiments described herein, one or several amino acids (e.g., 1, 2, 3 or 4) at the amino or carboxy terminus of the light and/or heavy chain, such as the C-terminal lysine of the heavy chain, may be missing or derivitized in some or all of the molecules in a composition. One specific example of such a modification, is an antibody or antigen binding fragment thereof in which the carboxy terminal lysine of the heavy chain is missing (e.g., as part of a post-translational modification). Furthermore, it should be understood that any of the sequences described herein include post-translational modifications to the specified sequence during expression of the antibody or antigen binding fragment thereof in cell culture (e.g., a CHO cell culture).

### 1. Humanized Antigen Binding Proteins

In certain embodiments, the antibody or antigen binding fragment thereof is a humanized antibody that binds CEACAM5. Typically, a non-human antibody is humanized to reduce immunogenicity to humans, while retaining the specificity and affinity of the parental non-human antibody. A humanized antibody is a genetically engineered antibody in which the HVRs (e.g., CDRs) or portions thereof from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557).

The acceptor antibody sequences can be, for example, a mature human antibody sequence, a composite of such sequences, a consensus sequence of human antibody sequences, or a germline region sequence. Human acceptor sequences can be selected for a high degree of sequence identity in the variable region frameworks with donor sequences to match canonical forms between acceptor and donor HVRs or CDRs among other criteria. Thus, a humanized antibody is an antibody having HVRs or CDRs entirely or substantially from a donor antibody and variable region framework sequences and constant regions, if present, entirely or substantially from human antibody sequences. Similarly, a humanized heavy chain typically has all three HVRs or CDRs entirely or substantially from a donor antibody heavy chain, and a heavy chain variable region framework sequence and heavy chain constant region, if present, substantially from human heavy chain variable region framework and constant region sequences. Likewise, a humanized light chain usually has all three CDRs entirely or substantially from a donor antibody light chain, and a light chain variable region framework sequence and light chain constant region, if present, substantially from human light chain variable region framework and constant region sequences. An HVR or CDR in a humanized antibody is substantially from a corresponding HVR or CDR in a non-human antibody when at least 80%, 85%, 90%, 95% or 100% of corresponding residues (as defined by Kabat) are identical between the respective HVRs or CDRs. The variable region framework sequences of an antibody chain or the constant region of an antibody chain are substantially from a human variable region framework sequence or human constant region respectively when at least 80%, 85%, 90%, 95% or 100% of corresponding residues defined by Kabat are identical.

Although humanized antibodies often incorporate all six HVRs (e.g., CDRs, preferably as defined by Kabat) from a mouse antibody, they can also be made with less than all HVRs or CDRs (e.g., at least 3, 4, or 5) HVRs or CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000).

Certain amino acids from the human variable region framework residues can be selected for substitution based on their possible influence on HVR (e.g.,CDR) conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to an HVR or CDR region,
(3) otherwise interacts with an HVR or CDR region (e.g. is within about 6 Å of such a region);
(4) mediates interaction between the heavy and light chains, or
(5) is the result of somatic mutation in the mouse chain.
(6) is a site of glycosylation.

Framework residues from classes (1)-(3) are sometimes alternately referred to as canonical and vernier residues. Canonical residues refer to framework residues defining the canonical class of the donor CDR loops determining the conformation of a CDR loop (Chothia and Lesk, J. Mol. Biol. 196, 901-917 (1987), Thornton & Martin, J. Mol. Biol., 263, 800-815, 1996). Vernier residues refer to a layer of framework residues that support antigen-binding loop conformations and play a role in fine-tuning the fit of an antibody to antigen (Foote & Winter, 1992, J Mol Bio. 224, 487-499).

Humanized antibodies and methods of making them are reviewed, e.g., in Almagro and Fransson, (2008) Front. Biosci. 13: 1619-1633, and are further described, e.g., in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34 (describing specificity determining region (SDR) grafting); Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that may be used for humanization include but are not limited to: framework regions selected using the “best-fit” method (see, e.g., Sims et al. (1993) J. Immunol. 151:2296); framework regions derived from the consensus sequence of human antibodies of a particular subgroup of light or heavy chain variable regions (see, e.g., Carter et al. (1992) Proc. Natl. Acad. Sci. USA, 89:4285; and Presta et al. (1993) J. Immunol, 151:2623); human mature (somatically mutated) framework regions or human germline framework regions (see, e.g., Almagro and Fransson, (2008) Front. Biosci. 13:1619-1633); and framework regions derived from screening FR libraries (see, e.g., Baca et al., (1997) J. Biol. Chem. 272: 10678-10684 and Rosok et al., (1996) J. Biol. Chem. 271:22611-22618).

### 2. Exemplary Antibody Constant Regions

In some embodiments, the heavy and light chain variable regions of antibodies described herein can be linked to at least a portion of a human constant region. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region. In some embodiments, an antibody described herein comprises a human IgG4 heavy chain constant region. In some of these embodiments, an antibody described herein comprises an S241P mutation in the human IgG4 constant region. In some embodiments, an antibody described herein comprises a human IgG4 constant region and a human κ light chain.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRn.

In some embodiments, one or several amino acids at the amino or carboxy terminus of the light and/or heavy chain, such as the C-terminal lysine of the heavy chain, may be missing or derivatized in a proportion or all of the molecules.

The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell-mediated effector functions than human IgG2 and IgG4. Light chain constant regions can be lambda or kappa.

Furthermore, as described in greater detail below, substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

### 3. Variants

The antigen binding proteins provided herein also include amino acid sequence variants of the antigen binding proteins provided herein. As an example, variants with improved binding affinity and/or other biological properties of the antibody can be prepared. Amino acid sequence variants of an antigen binding protein can be prepared by introducing appropriate modifications into the nucleotide sequence encoding the antigen binding protein, or by peptide synthesis. Such modifications include, for example, deletions from, and/or insertions into and/or substitutions of residues within the amino acid sequences of the antigen binding protein. Any combination of deletion, insertion, and substitution can be made to arrive at the final construct, provided that the final construct possesses the desired characteristics, e.g., antigen-binding.

In some embodiments, an antigen binding protein is a variant in that it has one or more amino acid substitutions, deletions and/or insertions relative to an antigen binding protein as described herein. In certain such embodiments, the variant has one or more amino acid substitutions. In further such embodiments, the substitutions are conservative amino acid substitutions.

An amino acid substitution can include but are not limited to the replacement of one amino acid in a polypeptide with another amino acid. Conservative amino acid substitutions can encompass non-naturally occurring amino acid residues, which are typically incorporated by chemical peptide synthesis rather than by synthesis in biological systems. Naturally occurring residues can be divided into classes based on common side chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

TABLE 3

| | | Preferred |
|---|---|---|
| Original Residue | Exemplary Substitutions | Substitutions |
| Ala | Val; Leu; Ile | Val |
| Arg | Lys; Gln; Asn | Lys |
| Asn | Gln; His; Asp; Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Pro; Ala | Ala |
| His | Asn; Gln; Lys; Arg | Arg |
| Ile | Leu; Val; Met; Ala; Phe; Norleucine | Leu |
| Leu | Norleucine; Ile; Val; Met; Ala; Phe | Ile |
| Lys | Arg; Gln; Asn | Arg |
| Met | Leu; Phe; Ile | Leu |
| Phe | Trp; Leu; Val; Ile; Ala; Tyr | Leu |
| Pro | Ala | Ala |
| Se | Thr; Ala; Cys | Thr |
| Thr | Val; Ser | Ser |
| Trp | Tyr; Phe | Tyr |
| Tyr | Trp; Phe; Thr; Ser | Phe |
| Val | Ile; Leu; Met; Phe; Ala; Norleucine | Leu |

Non-conservative substitutions involve exchanging a member of one of these classes for another class.

In altering the amino acid sequence of the antigen binding protein (e.g., anti-CEACAM5 antibody), in some embodiments the hydropathic index of amino acids can be considered. Each amino acid has been assigned a hydropathic index on the basis of its hydrophobicity and charge characteristics as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., 1982, J. Mol. Biol., 157:105−131. It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide (e.g., antibody) thus created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Alterations (e.g., substitutions) can be made in HVRs, e.g., to improve antibody affinity. Such alterations can be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions can occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, an antibody variant is prepared that has improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an antibody variant comprises an Fc region with one or more amino acid substitutions which improve ADCC, e.g., substitutions at positions 298, 333, and/or 334 of the Fc region (EU numbering of residues). For instance, a systemic substitution of solvent-exposed amino acids of human IgG1 Fc region has generated IgG variants with altered FcγR binding affinities (Shields et al., 2001, J. Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J. Mol. Biol. 336:1239-49).

In some embodiments, alterations are made in the Fc region to alter (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000). For instance, complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J. Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

The half-life of an antibody as provided herein can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG₁ involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J. Biol. Chem. 276:6591-604). IgG1 molecules harboring these substitutions have longer serum half-lives. Consequently, these modified IgG₁ molecules may be able to carry out their effector functions, and hence exert their therapeutic efficacies, over a longer period of time compared to unmodified IgG₁. Other exemplary substitutions for increasing binding to FcRn include a Gln at position 250 and/or a Leu at position 428. Other studies have shown that binding of the Fc region to FcRn can be improved by introducing one or more substitutions at one or more the following Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (see, e.g., U.S. Pat. Nos. 7,371,826; and 7,361,740).

In certain embodiments, an antibody as provided herein includes one or more modifications so as to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure.

Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity. Antibodies including such substitutions or engineering are included in some of the embodiments provided herein.

In certain embodiments, antibodies are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Other antibodies are further provided which contain bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In some embodiments, an antibody variant as provided herein includes a substitution of the native amino acid to a cysteine residue at amino acid position 234, 235, 237, 239, 267, 298, 299, 326, 330, or 332, preferably an S239C mutation (substitutions of the constant regions are according to the EU index) in a human IgG1 isotype. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (e.g., coupling cytotoxic drugs using thiol specific reagents such as maleimide derivatives of drugs). The presence of a therapeutic agent causes steric hindrance, thereby further reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821.)

In other cysteine engineered antibody variants, one or more reactive thiol groups are positioned at accessible sites of the antibody and can be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Generating of cysteine engineered antibodies are described, e.g., in U.S. Pat. No. 7,521,541.

Certain of the antibody or antigen binding fragment thereof that are provided include the following modifications to the constant region

The antigen binding proteins provided herein include those that compete with one of the exemplified antibody or antigen binding fragment thereof described above for specific binding to CEACAM5. In some of these embodiments, the test and reference antibody or antigen binding fragment thereof cross-compete with one another. Such antibody or antigen binding fragment thereof may bind to the same epitope as one of the antigen binding proteins described herein, or to an overlapping epitope. Antibody or antigen binding fragment thereof including fragments that compete with the exemplified antibodies are expected to show similar functional properties (e.g., one or more of the activities described above). The exemplified antibody or antigen binding fragment thereof and fragments include those described above, including those with: 1) the heavy and/or light chains, 2) VHs and/or VLs, and/or 3) that comprise one or more of the CDRs provided herein.

Thus, in some embodiments, the antibody or antigen binding fragment thereof that are provided include those that compete with an antibody having: all 6 of the CDRs listed for the same antibody provided herein.

In any of the embodiments described in this section, the antibody or antigen binding fragment thereof can have any combination or all of the activities listed herein. In further such embodiments, the test and reference antibody or antigen binding fragment thereof cross-compete with one another.

In some embodiments, competition or cross-competition is determined by surface plasmon resonance analysis (e.g., BIACORE®) (see, e.g., Abdiche, et al., 2009, Anal. Biochem. 386:172-180; Abdiche, et al., 2012, J. Immunol Methods 382:101-116; and Abdiche, et al., 2014 PLoS One 9:e92451

In another embodiment, the antigen binding proteins that are provided include those that bind the same epitope as any of the antibody or antigen binding fragment thereof described herein. A variety of techniques are available to identify antibodies or antigen binding fragments thereof that bind to the same epitope as one or more of the antibodies or antigen binding fragments thereof described herein. Such methods include, for instance, competition assays such as described herein, screening of peptide fragments, MS-based protein footprinting, alanine or glutamine scanning approaches, and via x-ray analysis of crystals of antigen:antigen binding protein complexes which provides atomic resolution of the epitope.

One approach for determining the epitope or epitope region (an “epitope region” is a region comprising the epitope or overlapping with the epitope) bound by a specific antibody involves assessing binding of an antibody or antigen binding fragment thereof to peptides comprising fragments of CEACAM5, e.g., non-denatured or denatured fragments. A series of overlapping peptides encompassing the sequence of CEACAM5 (e.g., human CEACAM5) can be prepared and screened for binding, e.g. in a direct ELISA, a competitive ELISA (where the peptide is assessed for its ability to prevent binding of an antibody to CEACAM5 bound to a well of a microtiter plate), or on a chip. Such peptide screening methods may not be capable of detecting some discontinuous functional epitopes, i.e. functional epitopes that involve amino acid residues that are not contiguous along the primary sequence of the CEACAM5 polypeptide chain.

In other embodiments, the region(s) containing residues that are in contact with or are buried by an antibody can be identified by mutating specific residues in CEACAM5 and determining whether the antibody or antigen binding fragment thereof can bind the mutated or variant CEACAM5 protein. By making a number of individual mutations, residues that play a direct role in binding or that are in sufficiently close proximity to the antibody such that a mutation can affect binding between the antigen binding protein and antigen can be identified. From a knowledge of these amino acids, the domain(s) or region(s) of the antigen that contain residues in contact with the antibody or antigen binding fragment thereof or covered by the antibody can be elucidated. Such a domain can include the binding epitope of an antibody. The general approach for such scanning techniques involves substituting arginine and/or glutamic acid residues (typically individually) for an amino acid in the wild-type polypeptide. These two amino acids are typically used in such scanning techniques because they are charged and bulky and thus have the potential to disrupt binding between an antibody and the CEACAM5 in the region of the CEACAM5 where the mutation is introduced. Arginines that exist in the wild-type antigen are replaced with glutamic acid. A variety of such individual mutants are obtained and the collected binding results analyzed to determine what residues affect binding (see, e.g., Nanevicz, T., et al., 1995, J. Biol. Chem., 270:37, 21619-21625 and Zupnick, A., et al., 2006, J. Biol. Chem., 281:29, 20464-20473).

An alternative approach for identifying an epitope is by MS-based protein footprinting, such as hydrogen/deuterium exchange mass spectrometry (HDX-MS) and Fast Photochemical Oxidation of Proteins (FPOP). Methods for conducting HDX-MS are described, for example, in Wei et al. (2014) Drug Discovery Today 19:95. Methods for performing FPOP are described, for instance, in Hambley and Gross (2005) J. American Soc. Mass Spectrometry 16:2057.

The epitope bound by an antibody or antigen binding fragment thereof can also be determined by structural methods, such as an X-ray crystal structure determination, molecular modeling, and nuclear magnetic resonance (NMR) spectroscopy, including NMR determination of the H-D exchange rates of labile amide hydrogens in the antigen when free and when bound in a complex with an antibody or antigen binding fragment thereof (see, e.g., Zinn-Justin et al. (1992) Biochemistry 31, 11335-11347; and Zinn-Justin et al. (1993) Biochemistry 32, 6884-6891).

X-ray crystallography analyses can be accomplished using any of the known methods in the art. Examples of crystallization methods are described, for instance, by Giege et al. (1994) Acta Crystallogr. D 50:339-350; and McPherson (1990) Eur. J. Biochem. 189:1-23). Such crystallization approaches include microbatch (e.g. Chayen (1997) Structure 5:1269-1274), hanging-drop vapor diffusion (e.g. McPherson (1976) J. Biol. Chem. 251:6300-6303), seeding and dialysis. Once formed, the antibody:antigen crystals themselves can be studied using well-known X-ray diffraction techniques and can be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al., eds., Academic Press; U.S. Patent Application Publication No. 2004/0014194), and BUSTER (Bricogne (1993) Acta Cryst. D 49:37-60; Bricogne (1997) Meth. Enzymol. 276A:361-423, Carter & Sweet, eds.; Roversi et al. (2000) Acta Cryst. D 56:1313-1323).

The antigen binding proteins in some embodiments bind to CEACAM5 with an affinity (e.g., EC₅₀) of less than 60 nM, 40 nM, 20 nM, 10 nM, 5 nM, 2 nM, 1 nM, 500 pM, 250 pM, 100 pM, 50 pM, 25 pM, 10 pM, or 1 pM. In some embodiments, the antibody or antigen binding fragment thereof binds to CEACAM5 with an affinity of between 5-10 nM, 1-5 nM, 500 pM-1 nM, 100-250 pM, 50-100 pM, 10-50 pM, or 1-10 pM.

### 4. Derivatives

In some embodiments, the antigen binding protein is a derivative of an antigen binding protein, such as those described herein are derivatized antigen binding proteins that can comprise any molecule or substance that imparts a desired property to the antigen binding protein (e.g., antibody or fragment), such as increased half-life in a particular use. The derivatized antigen binding protein can comprise, for example, a detectable (or labeling) moiety (e.g., a radioactive, colorimetric, antigenic or enzymatic molecule, or a detectable bead (such as a magnetic or electrodense (e.g., gold) bead); a molecule that binds to another molecule (e.g., biotin or streptavidin); a therapeutic or diagnostic moiety (e.g., a radioactive, cytotoxic, or pharmaceutically active moiety); or a molecule that increases the suitability of the antigen binding protein for a particular use (e.g., administration to a subject, such as a human subject, or other in vivo or in vitro uses). Examples of molecules that can be used to derivatize an antigen binding protein include albumin (e.g., human serum albumin) and polyethylene glycol (PEG). Albumin-linked and PEGylated derivatives of antigen binding proteins can be prepared using techniques well known in the art.

Other derivatives include covalent or aggregative conjugates of antigen binding proteins with other proteins or polypeptides, such as by expression of recombinant fusion proteins comprising heterologous polypeptides fused to the N-terminus or C-terminus of the antigen binding protein. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of the antigen binding protein (e.g., poly-His, or a FLAG peptide).

### 5. Oligomers

Oligomers that contain one or more antigen binding proteins are also provided. Oligomers can be in the form of covalently-linked or non-covalently-linked dimers, trimers, or higher oligomers. In an embodiment, oligomers comprising two or more antigen binding proteins are provided, with one example being a homodimer. Other oligomers include heterodimers, homotrimers, heterotrimers, homotetramers, heterotetramers and the like.

One embodiment is directed to oligomers comprising multiple CEACAM5 antigen binding polypeptides joined via covalent or non-covalent interactions between peptide moieties fused to the CEACAM5 antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular embodiments, the oligomers comprise from two to four CEACAM5 antigen binding proteins. The CEACAM5 antigen binding protein moieties of the oligomer may be in any of the forms described above, e.g., variants or fragments.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, Proc. Natl. Acad. Sci. USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

In another embodiment, the antigen binding protein is a dimer created by fusing a CEACAM5 antigen binding protein to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

Alternatively, the oligomer is a fusion protein comprising multiple CEACAM5 antigen binding proteins, with or without peptide linkers (spacer peptides). Among the suitable peptide linkers are those described in U.S. Pat. Nos. 4,751,180 and 4,935,233.

Another method for preparing oligomeric CEACAM5 antigen binding protein oligomers involves use of a leucine zipper. Leucine zipper domains are peptides that promote oligomerization of the proteins in which they are found (Landschulz et al., 1988, Science 240:1759). Among the known leucine zippers are naturally-occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-278. In one approach, recombinant fusion proteins comprising a CEACAM5 antigen binding protein fragment or derivative fused to a leucine zipper peptide are expressed in suitable host cells, and the soluble oligomeric CEACAM5 antigen binding protein fragments or derivatives that form are recovered from the culture supernatant.

### 6. Multispecific Antigen Binding Proteins

In a further aspect, the antibody or antigen binding fragment thereof can be a multispecific antibody or antigen binding fragment thereof, e.g, a multispecific antibody such as a bispecific antibody. In certain embodiments, a multispecific antibody or antigen binding fragment thereof is a multispecific antibody that has binding specificity for at least two different targets. In some of these embodiments, one of the binding specificities is for CEACAM5 and the other is for a different antigen. In other embodiments, the bispecific antibody binds to two different epitopes of CEACAM5. In some embodiments, the bispecific antibody binds an antigen on a target cells and can be used to localize cytotoxic agents to cells expressing CEACAM5. Bispecific antibodies can be prepared as full-length antibodies or antibody fragments.

A variety of techniques for making multispecific antibodies can be utilized, including for example, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies can also be made by engineering electrostatic steering effects for making antibody Fc-heterodimeric molecules (WO 2009/089004A1); cross linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); and using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Exemplary bispecific antibody molecules as provided herein comprise (i) two antibodies one with a specificity to CEACAM5 and another to a second target that are conjugated together, (ii) a single antibody that has one chain specific to CEACAM5 and a second chain specific to a second molecule, and (iii) a single chain antibody that has specificity to CEACAM5 and a second molecule. In certain embodiments, the second target/second molecule is a target other than CEACAM5. In another embodiment, however, the second target is a different region or epitope on CEACAM5 such that the bispecific antibody binds two different epitopes on CEACAM5.

### 7. Other Exemplary Formats

An antigen binding protein (e.g., an antibody or antigen-binding fragment thereof) can be a single polypeptide, or can include two, three, four, five, six, seven, eight, nine, or ten (the same or different) polypeptides. In some embodiments where the antibody or antigen-binding fragment thereof is a single polypeptide, the antibody or antigen-binding fragment can include a single antigen-binding domain or two antigen-binding domains. In some embodiments where the antibody or antigen-binding fragment is a single polypeptide and includes two antigen-binding domains, the first and second antigen-binding domains can be identical or different from each other (and can specifically bind to the same or different antigens or epitopes).

The different parts of the antigen binding proteins described herein, such as the variable domains of the antibodies described herein can arranged in various configurations to obtain additional antigen binding proteins. For example, in some embodiments where the antibody or the antigen-binding fragment is a single polypeptide, the first antigen-binding domain and the second antigen-binding domain (if present) can each be independently selected from the group of: a VH domain, a VHH domain, a VNAR domain, and a scFv. In some embodiments where the antibody or the antigen-binding fragment is a single polypeptide, the antibody or antigen-binding fragment can be a BiTE®, a (scFv)₂, a nanobody, a nanobody-HSA, a DART, a TandAb, a scDiabody, a scDiabody-CH₃, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the antibody or antigen-binding fragment is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of VHH domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the antibody or antigen-binding fragment is a single polypeptide and includes two antigen-binding domains, the first antigen-binding domain and the second antigen-binding domain can both be VHH domains, or at least one antigen-binding domain can be a VHH domain. In some embodiments where the antibody or antigen-binding fragment is a single polypeptide and includes two antigen-binding domains, the first antigen-binding domain and the second antigen-binding domain are both V_{NAR} domains, or at least one antigen-binding domain is a V_{NAR} domain. In some embodiments where the antibody or antigen-binding domain is a single polypeptide, the first antigen-binding domain is a scFv domain. In some embodiments where the antibody or antigen-binding fragment is a single polypeptide and includes two antigen-binding domains, the first antigen-binding domain and the second antigen-binding domain can both be scFv domains, or at least one antigen-binding domain can be a scFv domain.

In some embodiments, the antibody or antigen-binding fragment can include two or more polypeptides (e.g., two, three, four, five, six, seven, eight, nine, or ten polypeptides). In some embodiments where the antibody or antigen-binding fragment includes two or more polypeptides, two, three, four, five or six of the polypeptides of the two or more polypeptides can be identical.

### B. Drugs and Linkers

A number of embodiments of the invention are described below, which are not meant to limit the invention in any way, are followed by a more detailed discussion of the components that make up the conjugates. One of skill in the art will understand that each of the conjugates identified and any of the selected embodiments thereof is meant to include the full scope of each component and linker.

In some embodiments, provided herein are ADCs having a formula:

L-(Q-D)ₚ

or a salt thereof, wherein
L is a Ligand Unit comprising an antibody or antigen binding fragment thereof that binds to CEACAM5;
the subscript p is an integer of from 1 to 16;
Q is a Linker Unit having a formula selected from the group consisting of:
—Z-A-, —Z-A-RL-; —Z-A-RL-Y—; Z-A-S*—W—; —Z-A-S*-RL-; —Z-A-B(S*)—RL-; —Z-A-S*—W-RL-, —Z-A-S*-RL-Y—; and —Z-A-B(S*)—RL-Y—;
wherein Z is a Stretcher Unit,
   A is a bond or a Connector Unit;

### B is a Parallel Connector Unit;

### S* is a Partitioning Agent;

### W is a Peptide Unit;

### RL is a Releasable Unit;

### Y is a Spacer Unit; and

D is a Drug Unit selected from the group consisting of:

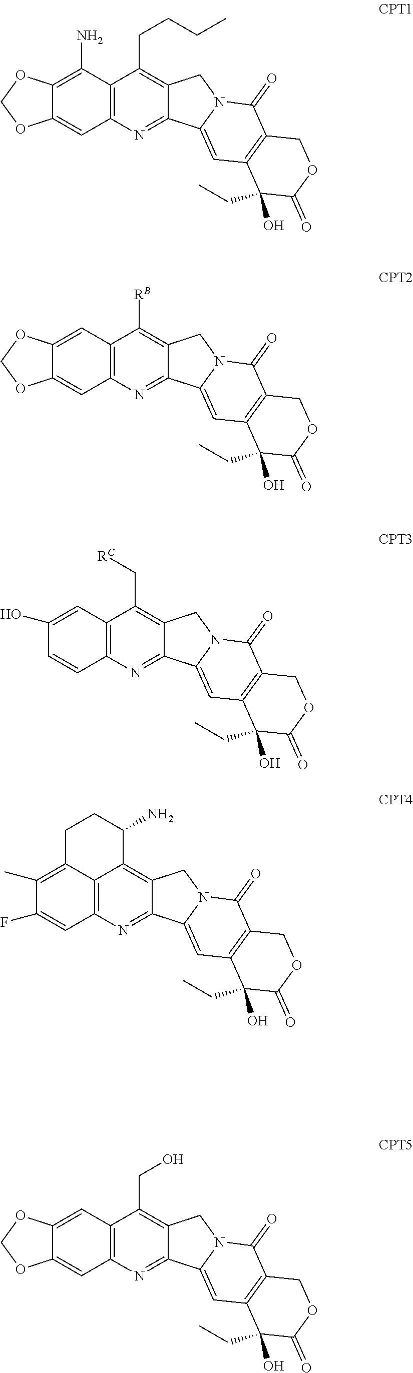

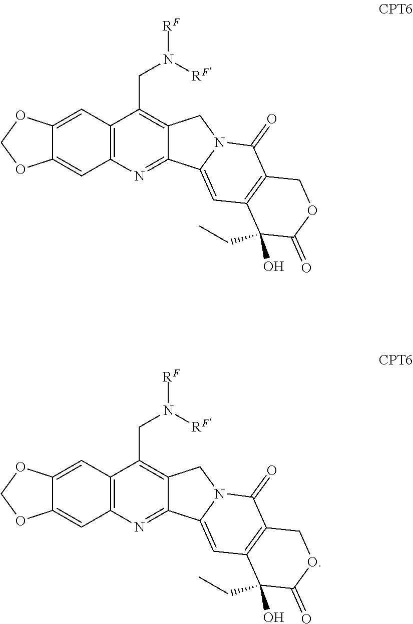

wherein R^{B} is a member selected from the group consisting of H, C₁-C₈ alkyl, C₁-C₈ haloalkyl, C₃-C₈ cycloalkyl, (C₃-C₈ cycloalkyl)-C₁-C₄ alkyl-, phenyl and phenyl-C₁-C₄ alkyl-;

R^{C} is a member selected from the group consisting of C₁-C₆ alkyl and C₃-C₆ cycloalkyl;

each R^{F} and R^{F′} is a member independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-, or

R^{F} and R^{F′} are combined with the nitrogen atom to which each is attached to form a 5-, 6- or 7-membered ring having 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and wherein the cycloalkyl, heterocycloalkyl, phenyl and heteroaryl portions of R^{B}, R^{C}, R^{F} and R^{F′} are substituted with from 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and

wherein the point of attachment of D to Q is through the heteroatom of any one of the hydroxyl or primary or secondary amine functional groups present on CPT1, CPT2, CPT3, CPT4, CPT5, CPT6 or CPT7 when Q is —Z-A-RL-, —Z-A-RL-Y—, —Z-A-S*-RL-, —Z-A-B(S*)—RL-, —Z-A-S*-RL-Y— or —Z-A-B(S*)—RL-Y— in which RL is any one of the Releasable Linkers disclosed herein, or

wherein the point of attachment of D to Q is through the oxygen atom of the hydroxyl group substituent in the lactone ring of CPT1, CPT2, CPT3, CPT4, CPT5, CPT6 or CPT7 when Q is —Z-A-, —Z-A-S*—W— or —Z-A-B(S*)—W—, or when Q is —Z-A-S*-RL-, —Z-A-B(S*)—RL-, —Z-A —S*—W-RL-, or —Z-A-B(S*)—W-RL- in which RL is a Releasable Unit other than a Glucuronide Unit; and

provided that at least one of R^{F} and R^{F′} is —H, when the point of attachment is to the nitrogen atom of the amino group of CPT6, and

provided that —Z-A-of —Z-A-RL-, —Z-A-RL-Y—, —Z A-S*-RL-, —Z-A-B(S*)—RL-, —Z-A-S*-RL-Y— and —Z-A-B(S*)—RL-Y— is other than succinimido-caproyl-β-alanyl, optionally having the succinimide ring in hydrolyzed form, when D is CPT1 having attachment through its amino group.

In one group of embodiments, D has formula CPT5.

In one group of embodiments, D has formula CPT2.

In one group of embodiments, D has formula CPT3.

In one group of embodiments, D has formula CPT4.

In one group of embodiments, D has formula CPT1.

In one group of embodiments, D has formula CPT6.

In one group of embodiments, D has formula CPT7.

In one group of embodiments, Q has a formula selected from the group consisting of:

—Z-A-RL- and —Z-A-RL-Y—,

wherein RL is a Releasable Linker that is a Glucuronide Unit and the groups Z, A and Y have the meanings provided above and in any one of the embodiments specifically recited herein.

In one group of embodiments, Q has a formula selected from the group consisting of:

—Z-A-S*-RL- and —Z-A-S*-RL-Y—,

wherein RL is a Releasable Linker that is a Glucuronide Unit and the groups Z, A, S* and Y have the meanings provided above and in any one of the embodiments specifically recited herein.

In one group of embodiments, Q has a formula selected from the group consisting of:

—Z-A-B(S*)—RL- and —Z-A-B(S*)—RL-Y—,

wherein RL is a Releasable Linker that is a Glucuronide Unit and the groups Z, A, S*, B and Y have the meanings provided above and in any one of the embodiments specifically recited herein.

In another group of embodiments, Q has a formula selected from the group consisting of:

—Z-A- or —Z-A-RL-,

wherein RL is a Releasable Linker that is other than a Glucuronide Unit and the groups Z and A have the meanings provided above and in any one of the embodiments specifically recited herein.

In another group of embodiments, Q has a formula selected from the group consisting of:

—Z-A-S*-RL- and —Z-A-B(S*)—RL-,

wherein RL is a Releasable Linker that is other than a Glucuronide Unit and the groups Z, A, S* and B have the meanings provided above and in any one of the embodiments specifically recited herein.

In another group of embodiments, Q has a formula selected from the group consisting of:

—Z-A-S*—W— and —Z-A-B(S*)—W—,

wherein the groups Z, A, S*, B and W have the meanings provided above and in any one of the embodiments specifically recited herein.

In another group of embodiments, Q has a formula selected from the group consisting of:

—Z-A-S*—W-RL- and —Z-A-B(S*)—W-RL-,

wherein RL is a Releasable Linker that is other than a Glucuronide Unit and the groups Z, A, S*, B and W have the meanings provided above and in any one of the embodiments specifically recited herein.

In one group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT1 are represented by formulae of:

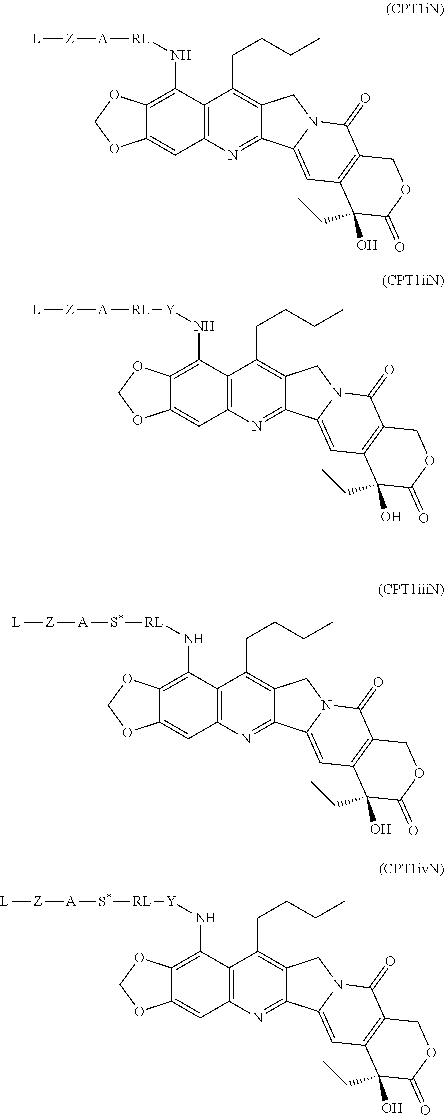

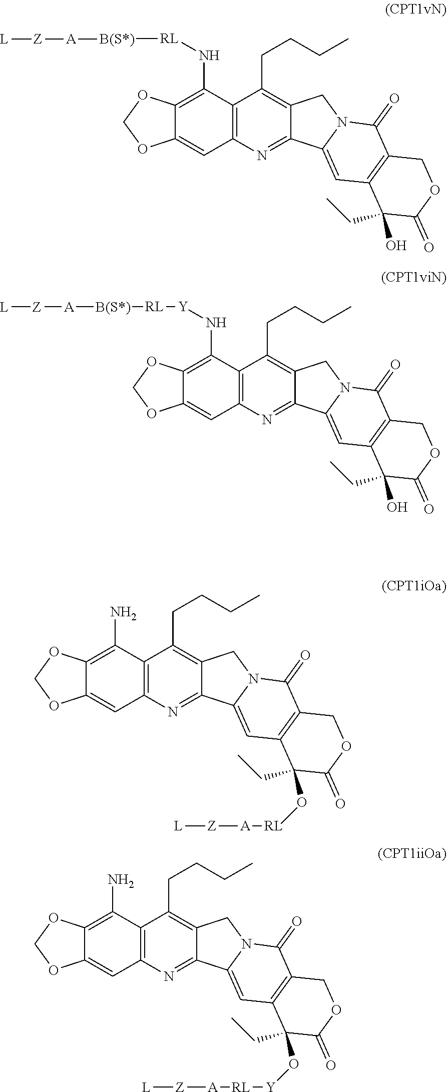

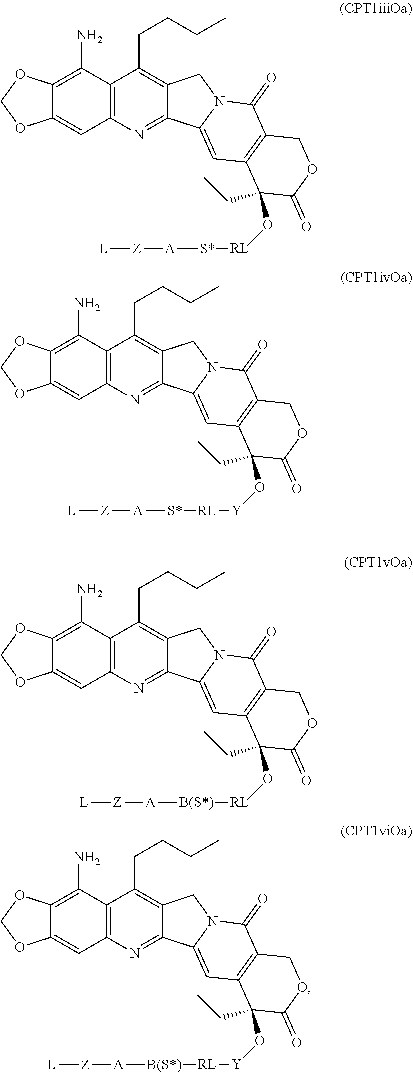

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein provided that —Z-A- of formula CPT1iN, CPT1iiN, CPT1iiiN, CPT1ivN, CPT1vN and CPT1viN is other than succinimido-caproyl-β-alanyl, optionally having the succinimide ring in hydrolyzed form.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- and are comprised of a Drug Unit having formula CPT1 are represented by formulae of:

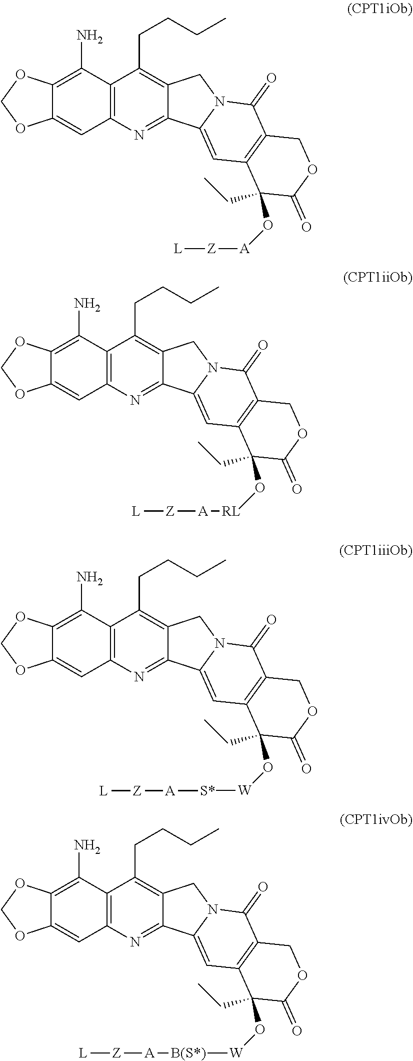

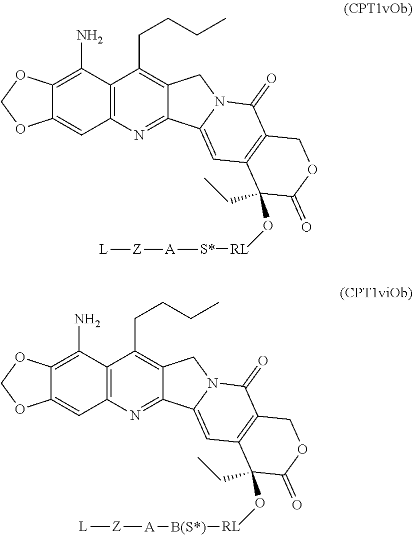

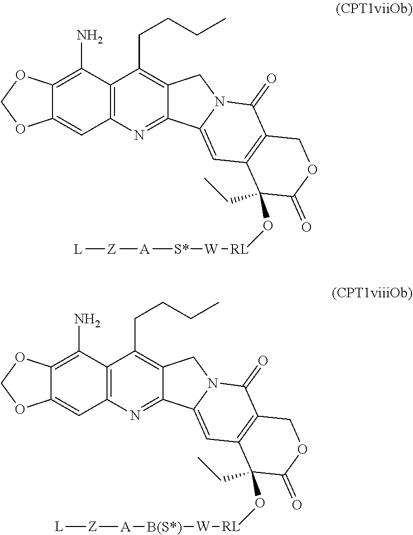

respectively, wherein RL is a Releasable Linker that is other than a Glucuronide Unit and the groups L, Z, A, S*, B and W have the meanings provided above and in any one of the embodiments specifically recited herein.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT2 are represented by the formulae of:

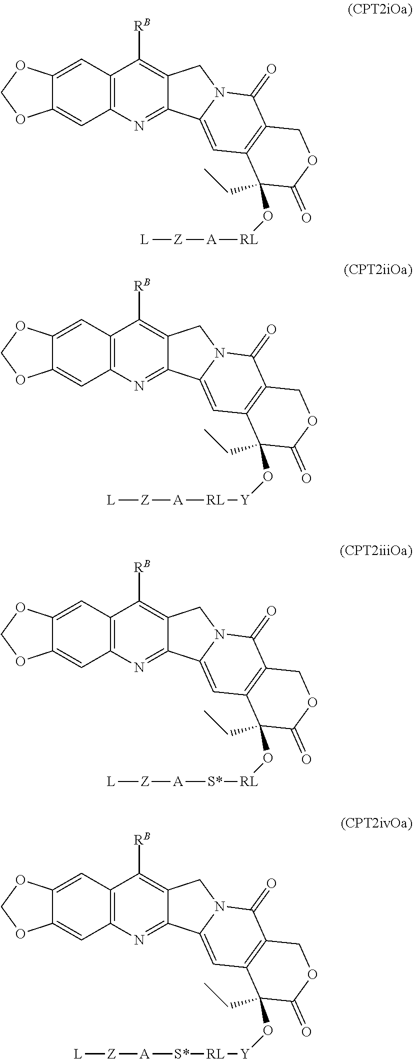

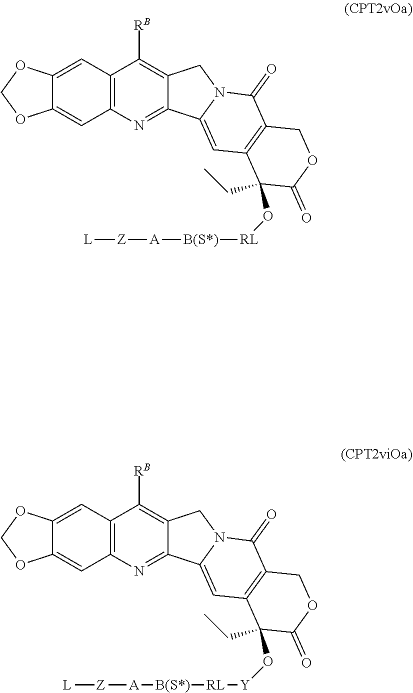

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- are comprised of a Drug Unit having formula CPT2 are represented by formulae of:

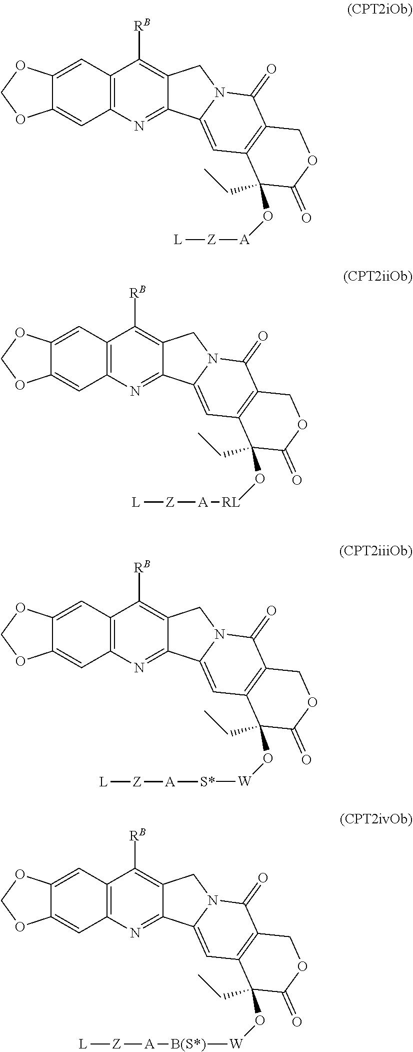

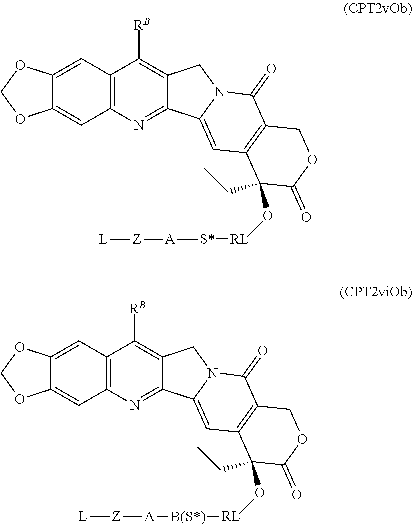

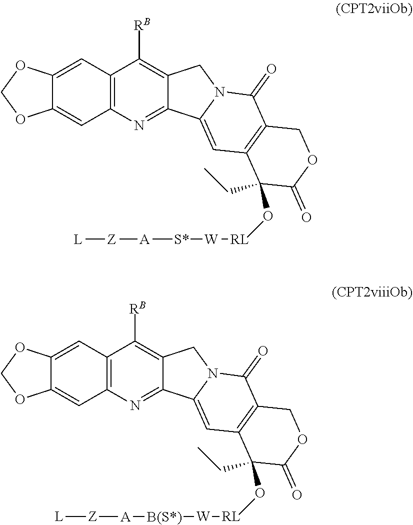

respectively, wherein RL is a Releasable Linker that is other than a Glucuronide Unit and the groups L, Z, A, S*, B and W have the meanings provided above and in any one of the embodiments specifically recited herein.

In one group of embodiments, R^{B} in formula CPT2iOa, CPT2iiOa, CPT2iiiOa, CPT2ivOa, CPT2vOa, CPT2viOa, CPT2iOb, CPT2iiOb, CPT2iiiOb, CPT2ivOb, CPT2vOb, CPT2viOb, CPT2viiOb or CPT2viiiOb is a moiety selected from the group consisting of —H, C₁-C₈ alkyl and C₁-C₈ haloalkyl.

In one group of embodiments, R^{B} in formula CPT2iOa, CPT2iiOa, CPT2iiiOa, CPT2ivOa, CPT2vOa, CPT2viOa, CPT2iOb, CPT2iiOb, CPT2iiiOb, CPT2ivOb, CPT2vOb, CPT2viOb, CPT2viiOb or CPT2viiiOb is a moiety selected from the group consisting of C₃-C₈ cycloalkyl, (C₃-C₈ cycloalkyl)-C₁-C₄ alkyl-, phenyl and phenyl-C₁-C₄ alkyl-, and wherein the cycloalkyl and phenyl moieties of R^{B} are substituted with 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT3 are represented by the formulae of:

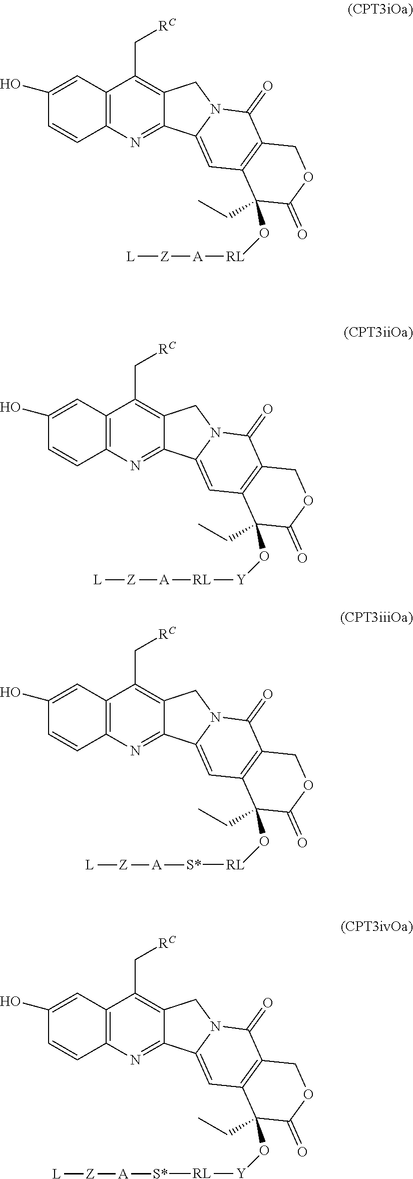

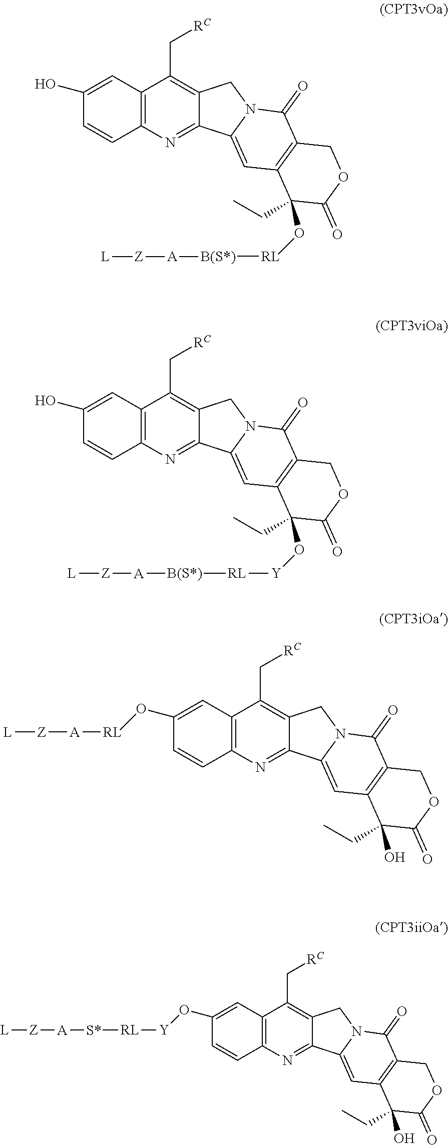

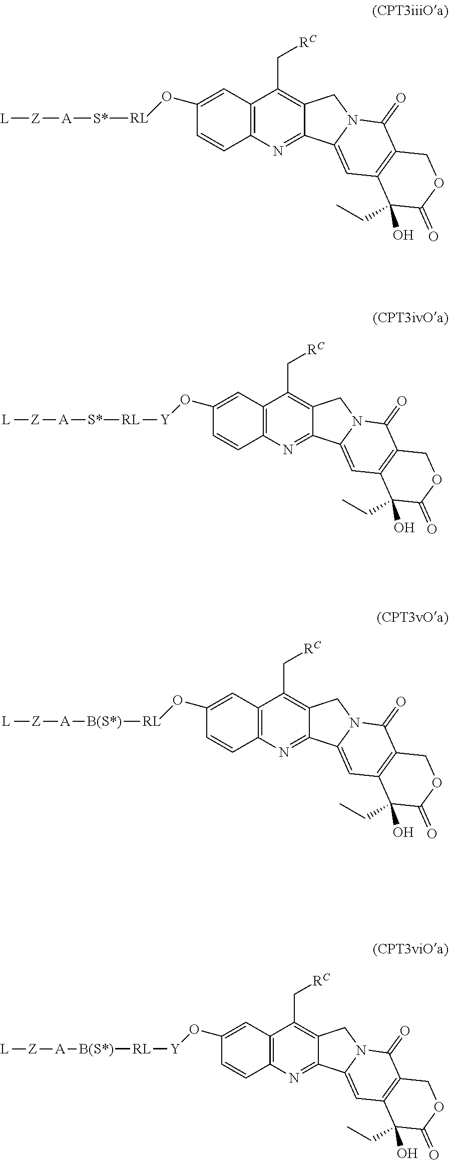

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- are comprised of a Drug Unit having formula CPT3 are represented by formulae of:

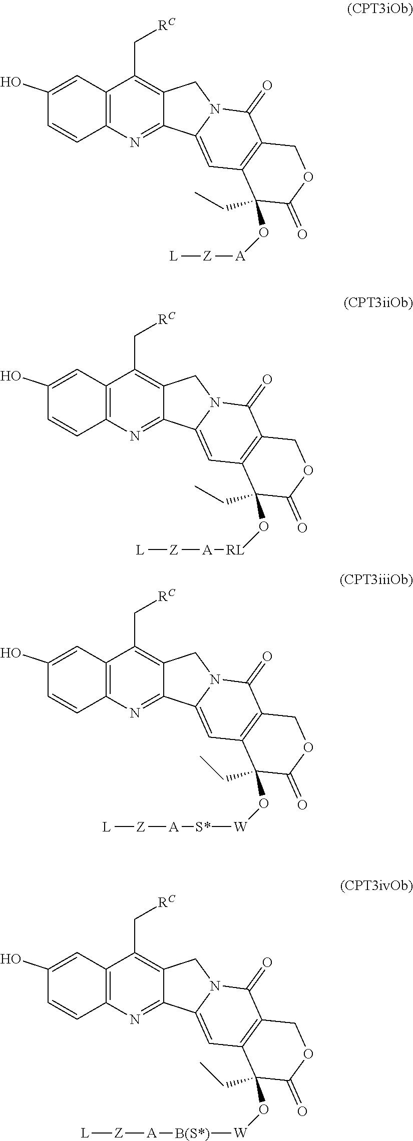

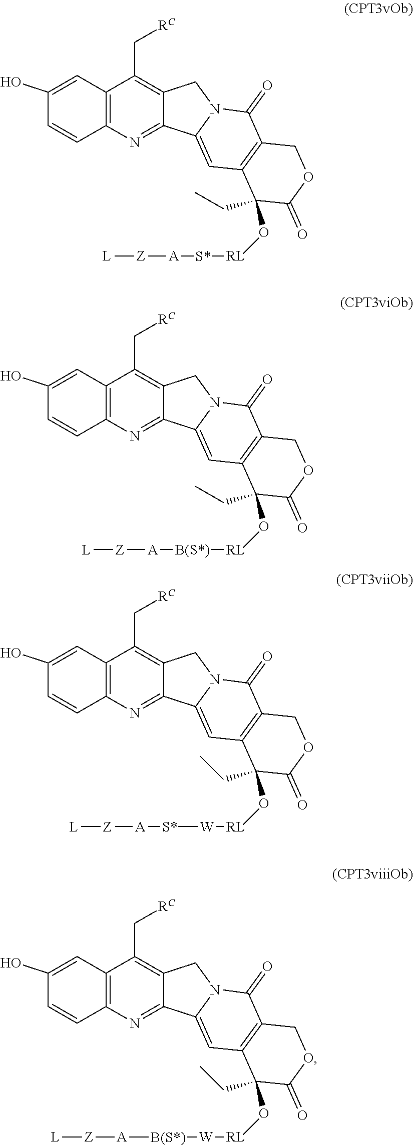

respectively, wherein RL is a Releasable Linker that is other than a Glucuronide Unit and the groups L, Z, A, S*, B and W have the meanings provided above and in any one of the embodiments specifically recited herein.

In one group of embodiments, R^{C} in formula CPT3iOa, CPT3iiOa, CPT3iiiOa, CPT3ivOa, CPT3vOa, CPT3viOa, CPT3iO′a, CPT3iiO′a, CPT3iiiO′a, CPT3ivO′a, CPT3vO′a, CPT3viO′a, CPT3iOb, CPT3iiOb, CPT3iiiOb, CPT3ivOb, CPT3vOb, CPT3viOb, CPT3viiOb or CPT3viiiOb is C₁-C₆ alkyl.

In one group of embodiments, R^{C} in formula CPT3iOa, CPT3iiOa, CPT3iiiOa, CPT3ivOa, CPT3vOa, CPT3viOa, CPT3iO′a, CPT3iiO′a, CPT3iiiO′a, CPT3ivO′a, CPT3vO′a, CPT3viO′a, CPT3iOb, CPT3iiOb, CPT3iiiOb, CPT3ivOb, CPT3vOb, CPT3viOb, CPT3viiOb or CPT3viiiOb is C₃-C₆ cycloalkyl.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT4, and are represented by the formulae of:

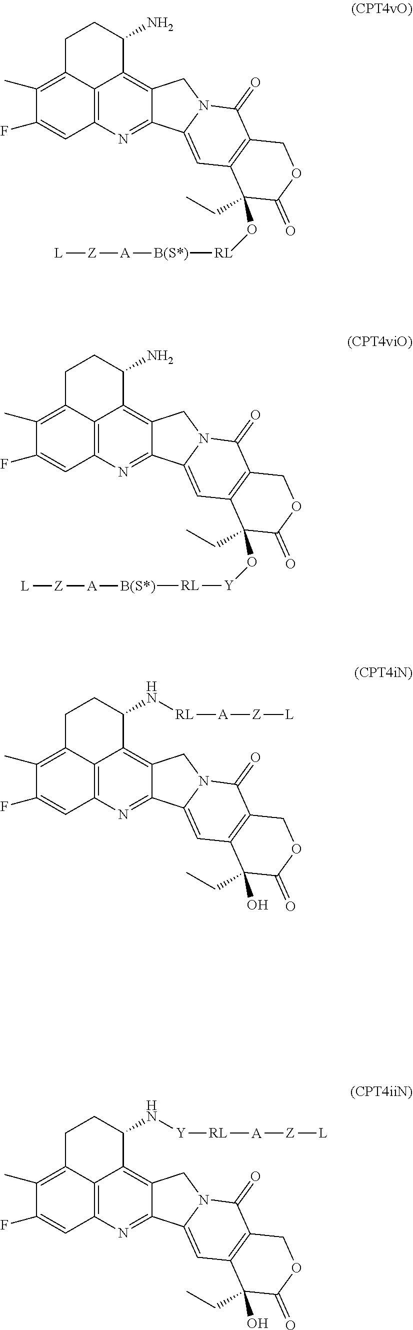

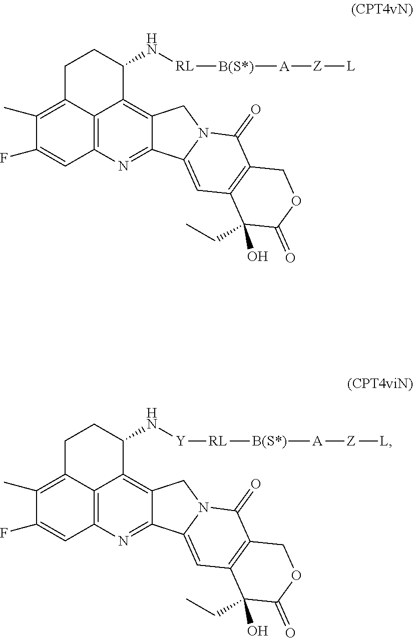

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein.

In other embodiments the ADCs in which Q has the formula of —Z-A-, —Z-A-RL-, —Z-A-S*—W—, —Z-A-B(S*)—, —Z-A-S*-RL-, —Z-A-B(S*)RL-, —Z-A-S*—W-RL- and —Z-A-B(S*)—W-RL- are comprised of a Drug Unit having formula CPT4 are represented by formulae of:

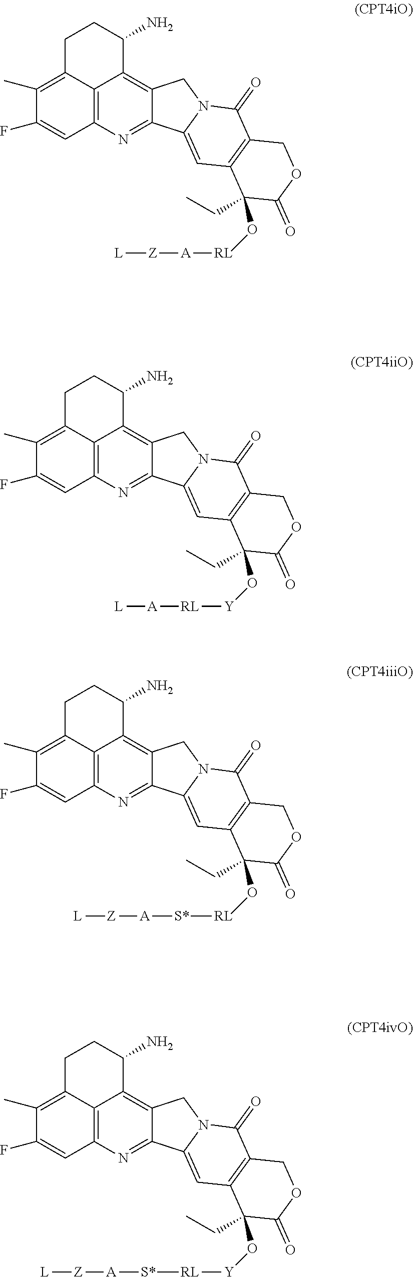

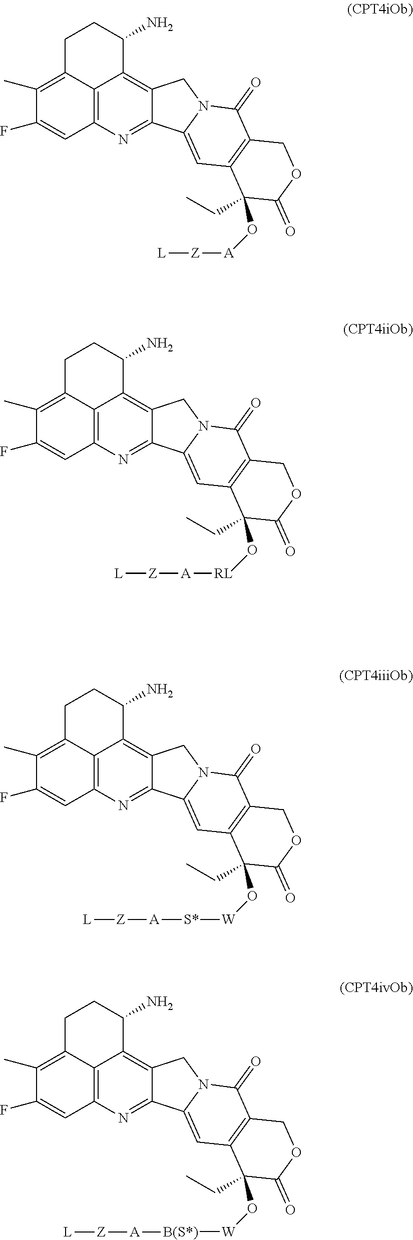

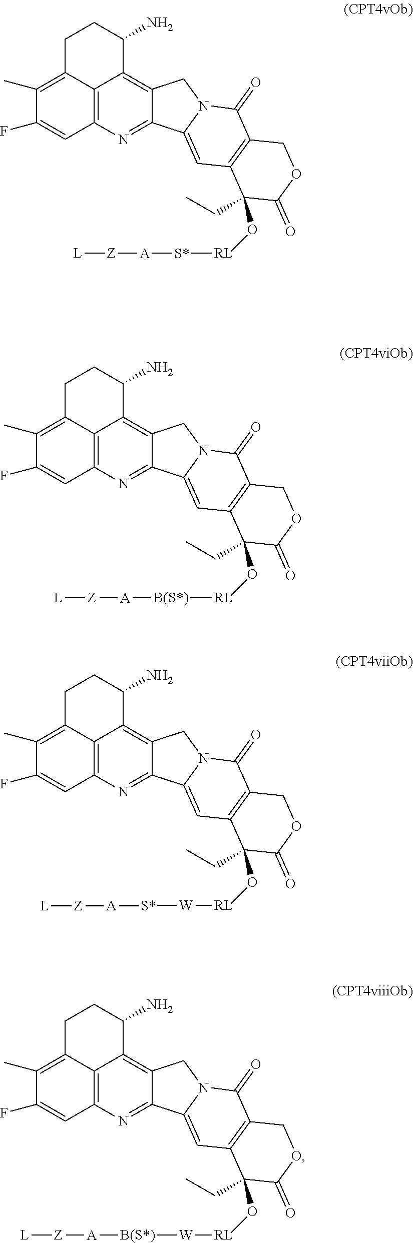

respectively, wherein RL is a Releasable Linker that is other than a Glucuronide Unit and the groups L, Z, A, S*, B and W have the meanings provided above and in any one of the embodiments specifically recited herein.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT5 are represented by the formulae of:

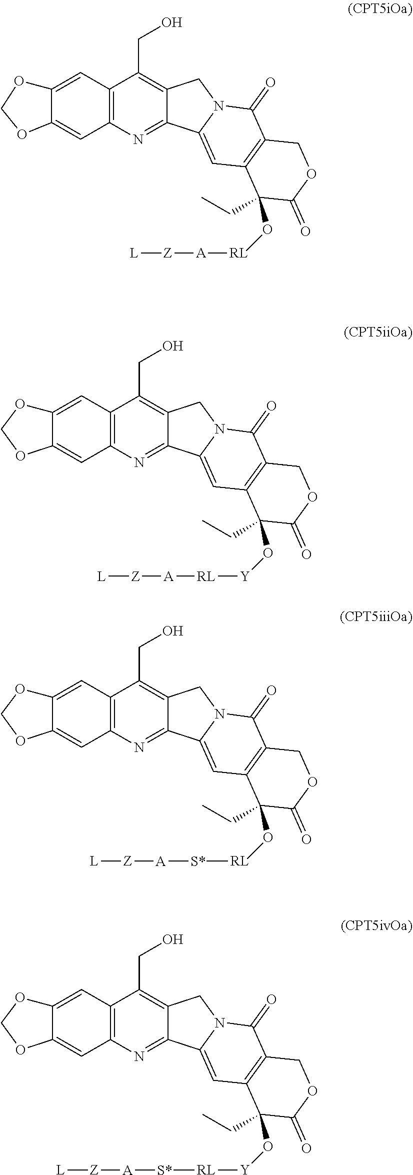

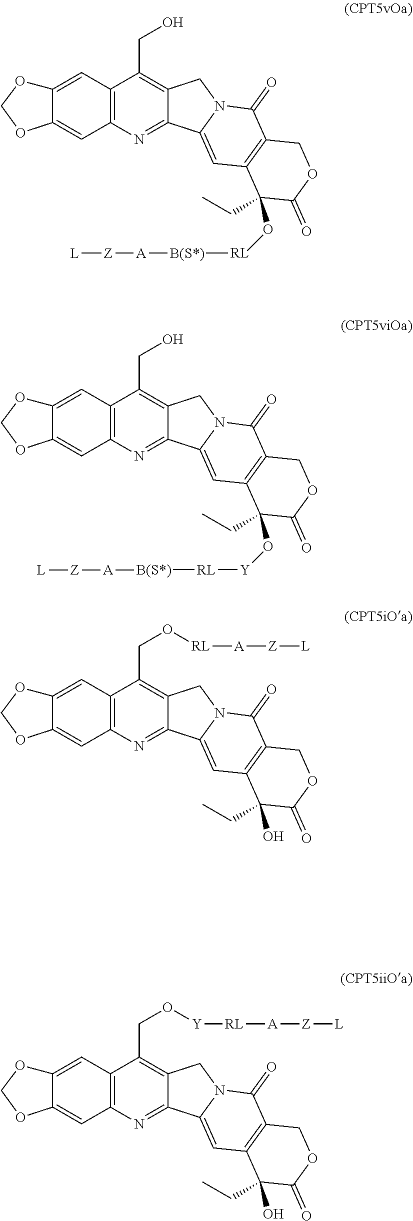

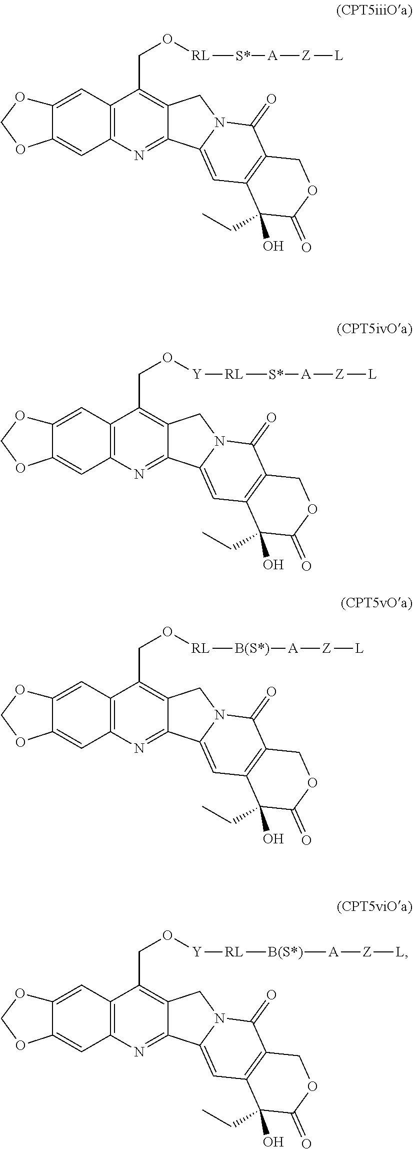

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- are comprised of a Drug Unit having formula CPT5 are represented by formulae of:

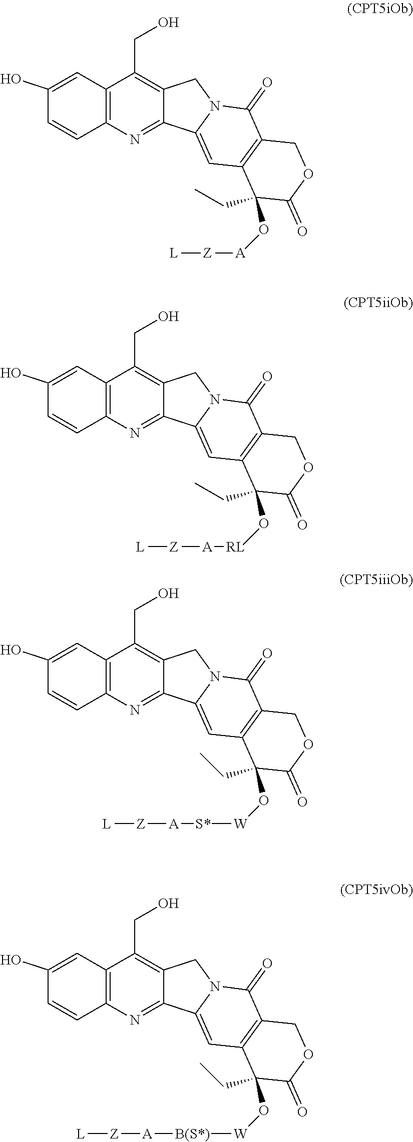

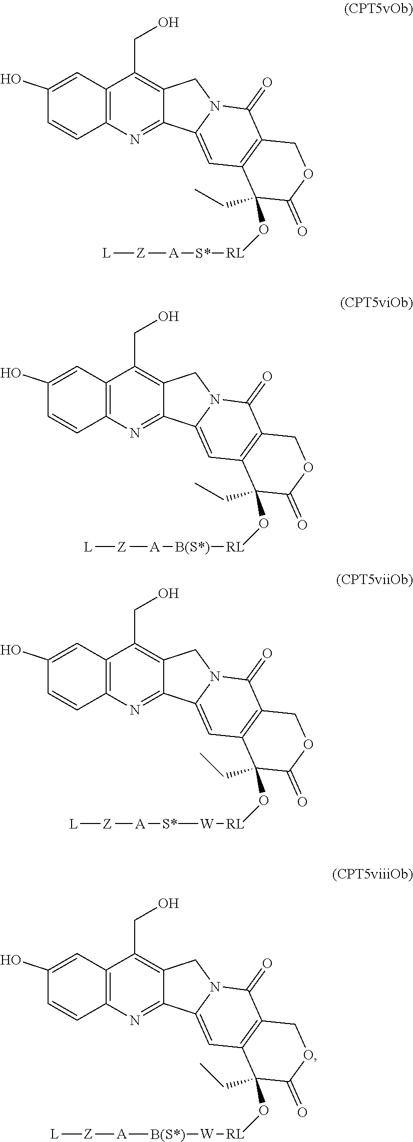

respectively, wherein RL is a Releasable Linker that is other than a Glucuronide Unit and the groups L, Z, A, S*, B and W have the meanings provided above and in any one of the embodiments specifically recited herein.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT6 are represented by the formulae of:

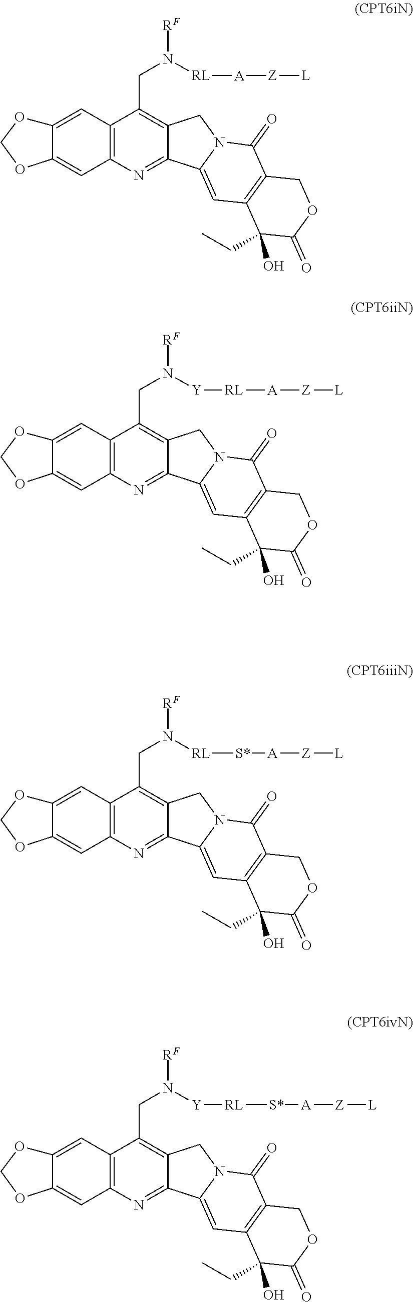

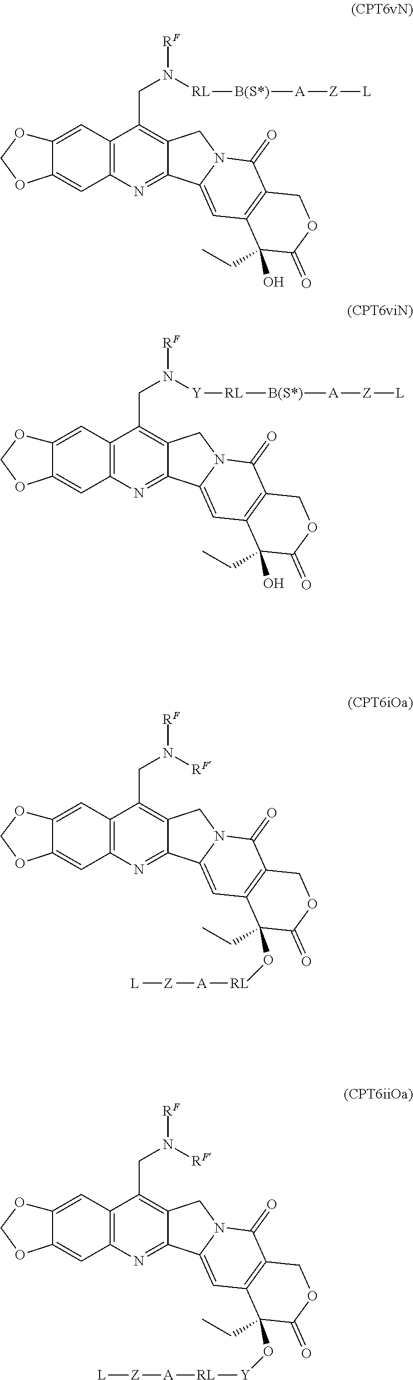

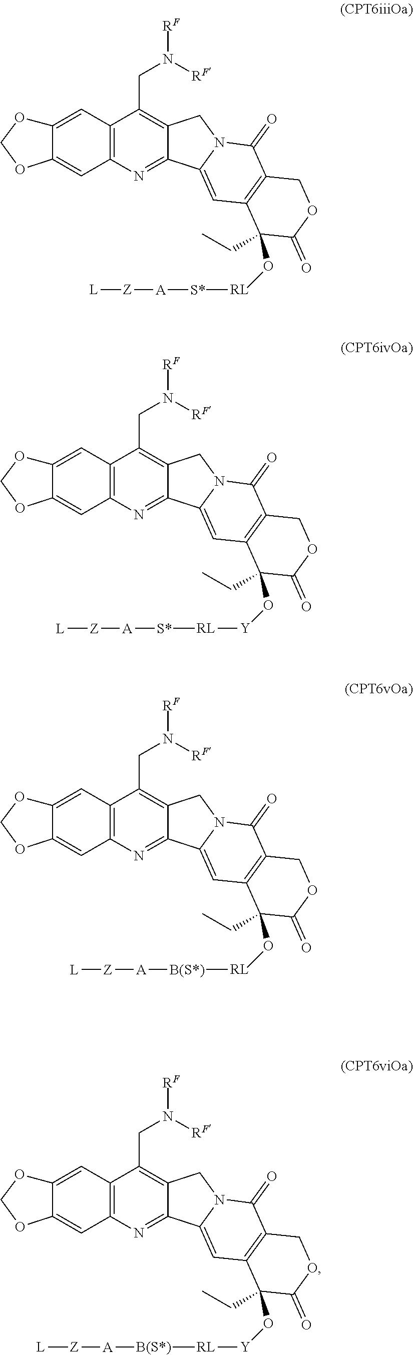

respectively, wherein RL is any one of the Releasable Linkers disclosed herein, preferably RL is a Glucuronide Unit, and the groups L, Z, A, S*, B and Y have the meanings provided above and in the any of the embodiments specifically recited herein.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- and are comprised of a Drug Unit having formula CPT6 are represented by formulae of:

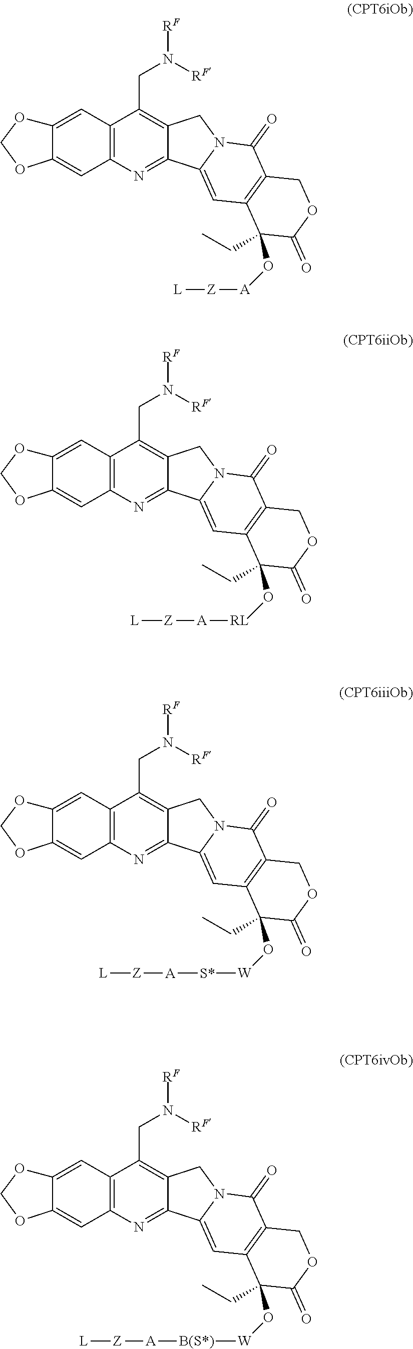

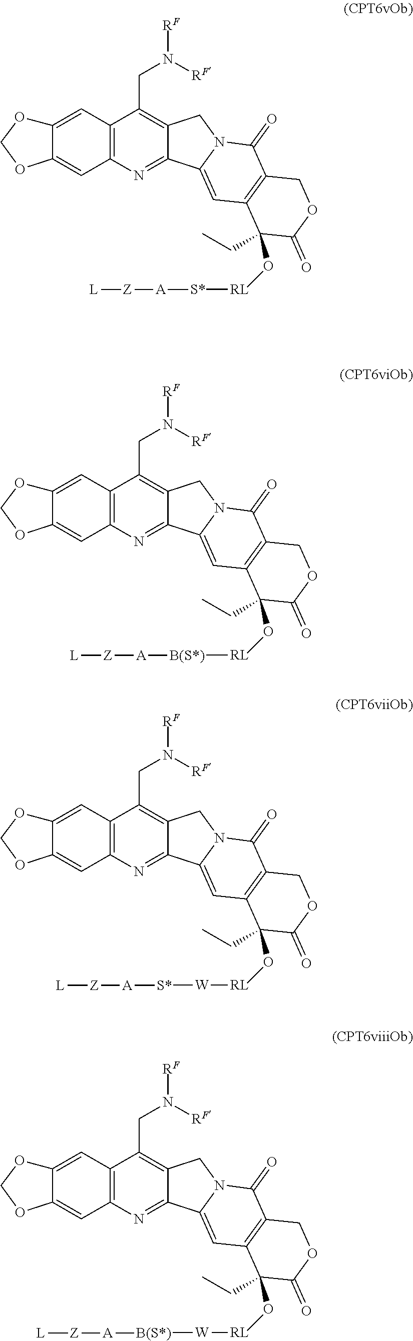

respectively, wherein RL is a Releasable Linker that is other than a Glucuronide Unit and the groups L, Z, A, S*, B and W have the meanings provided above and in any one of the embodiments specifically recited herein.

In one group of embodiments, R^{F} in formula CPT6iN, CPT6iiN, CPT6iiiN, CPT6ivN, CPT6vN or CPT6viN is —H.

In one group of embodiments, both R^{F} and R^{F′} in formula CPT6iOa, CPT6iiOa, CPT6iiiOa, CPT6ivOa, CPT6vOa, CPT6viOa, CPT6iOb, CPT6iiOb, CPT6iiiOb, CPT6ivOb, CPT6vOb, CPT6viOb, CPT6viiOb or CPT6viiiOb is —H.

In one group of embodiments, R^{F} in formula CPT6iN, CPT6iiN, CPT6iiiN, CPT6ivN, CPT6vN or CPT6viN is a moiety selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydoxyalkyl-C(O)—, and C₁-C₈ aminoalkylC(O)—.

In one group of embodiments, R^{F} in formula CPT6iN, CPT6iiN, CPT6iiiN, CPT6ivN, CPT6vN or CPT6viN is a moiety selected from the group consisting of C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl C₁-C₄ alkyl-, heteroaryl and heteroaryl-C₁-C₄ alkyl-, and wherein cycloalkyl, heterocycloalkyl, phenyl and heteroaryl moieties of R^{F} are substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂.

In one group of embodiments, R^{F} in formula CPT6iN, CPT6iiN, CPT6iiiN, CPT6ivN, CPT6vN or CPT6viN is a moiety independently selected from the group consisting of —H, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl, phenyl, phenyl-C₁-C₄ alkyl-, diphenyl C₁-C₄ alkyl, heteroaryl and heteroaryl-C₁-C₄ alkyl-, and wherein cycloalkyl, heterocycloalkyl, phenyl and heteroaryl moieties of R^{F} are substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂.

In one group of embodiments, R^{F} and R^{F′} in formula CPT6iOa, CPT6iiOa, CPT6iiiOa, CPT6ivOa, CPT6vOa, CPT6viOa, CPT6iOb, CPT6iiOb, CPT6iiiOb, CPT6ivOb, CPT6vOb, CPT6viOb, CPT6viiOb or CPT6viiiOb are combined with the nitrogen atom to which both are attached to form a 5-, 6- or 7-membered ring having 0 to 3 substituents selected independently from the group consisting of halogen, C₁-C₄ alkyl, —OH, —OC₁-C₄ alkyl, —NH₂, —NHC₁-C₄ alkyl and —N(C₁-C₄ alkyl)₂.

In one group of embodiments, at least one of R^{F} and R^{F′} in formula CPT6iOa, CPT6iiOa, CPT6iiiOa, CPT6ivOa, CPT6vOa, CPT6viOa, CPT6iOb, CPT6iiOb, CPT6iiiOb, CPT6ivOb, CPT6vOb, CPT6viOb, CPT6viiOb or CPT6viiiOb is a moiety independently selected from the group consisting of C₁-C₈ alkyl, C₁-C₈ hydroxyalkyl, C₁-C₈ aminoalkyl, (C₁-C₄ alkylamino)-C₁-C₈ alkyl, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkylC(O)—, C₁-C₈ hydoxyalkyl-C(O)—, and C₁-C₈ aminoalkyl-C(O)— and the other is a moiety selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydoxyalkyl-C(O)—, and C₁-C₈ aminoalkylC(O)—.

In one group of embodiments, each R^{F} and R^{F′} in formula CPT6iO, CPT6iiO, CPT6iiiO, CPT6ivO, CPT6vO or CPT6viO is a moiety independently selected from the group consisting of C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, and C₁-C₈ aminoalkyl-C(O)—.

In one group of embodiments, at least one of R^{F} and R^{F′} in formula CPT6iO, CPT6iiO, CPT6iiiO, CPT6ivO, CPT6vO or CPT6viO is a moiety independently selected from the group consisting of C₃-C₁₀ cycloalkyl, C₃-C₁₀ cycloalkyl-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl, diphenyl C₁-C₄ alkyl, heteroaryl and heteroaryl-C₁-C₄ alkyl-, and wherein the cycloalkyl, heterocycloalkyl, phenyl and heteroaryl moieties of R^{F} or R^{F′} are substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂, and the other is a moiety selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl-, C₁-C₈ alkyl-C(O)—, C₁-C₈ hydoxyalkyl-C(O)—, and C₁-C₈ aminoalkylC(O)—)₂.

In one group of embodiments, at least one of R^{F} and R^{F′} in formula CPT6iO, CPT6iiO, CPT6iiiO, CPT6ivO, CPT6vO or CPT6viO is a moiety independently selected from the group consisting of C₃-C₁₀ cycloalkyl, C₃-C₁₀ cycloalkyl-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl, diphenyl C₁-C₄ alkyl, heteroaryl and heteroaryl-C₁-C₄ alkyl-, and the other is a moiety selected from the group consisting of —H, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl C₁-C₄ alkyl-, heteroaryl and heteroaryl-C₁-C₄ alkyl-, wherein the cycloalkyl, heterocycloalkyl, phenyl and heteroaryl moieties of R^{F} and R^{F′} are independently substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂,

In one group of embodiments, each R^{F} and R^{F′} in formula CPT6iO, CPT6iiO, CPT6iiiO, CPT6ivO, CPT6vO or CPT6viO is a moiety independently selected from the group consisting of —H, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl C₁-C₄ alkyl-, heteroaryl and heteroaryl-C₁-C₄ alkyl-, and wherein the cycloalkyl, heterocycloalkyl, phenyl and heteroaryl moieties of R^{F} and R^{F′} are independently substituted with 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂.

In another group of embodiments, the ADCs in which Q has the formula of —Z-A-RL-, —Z-A-RL-Y—, —Z-A-S*-RL-, —Z-A-S*-RL-Y—, —Z-A-B(S*)—RL- or —Z-A-B(S*)—RL-Y— and are comprised of a Drug Unit having formula CPT7 are represented by the formulae of:

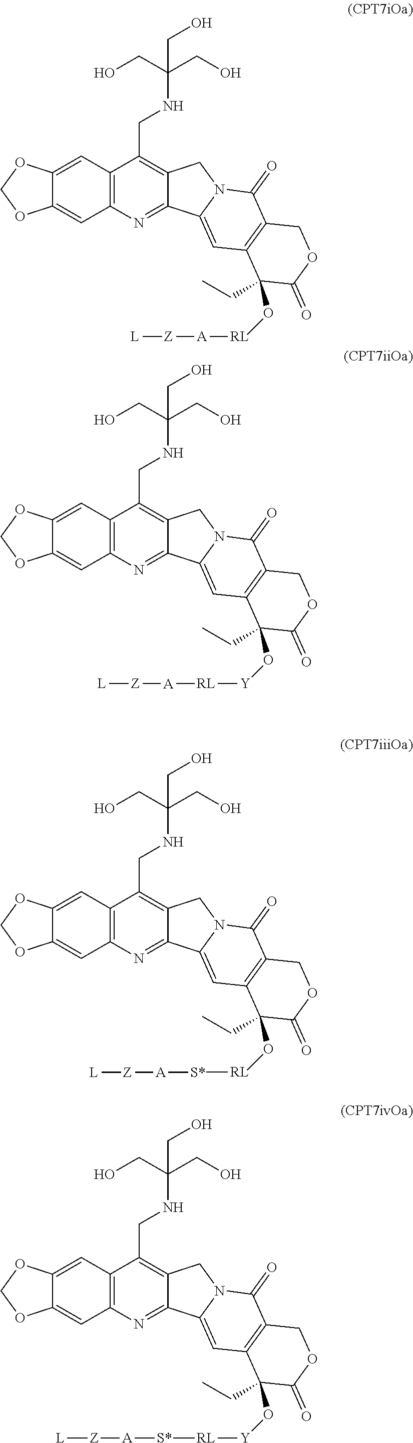

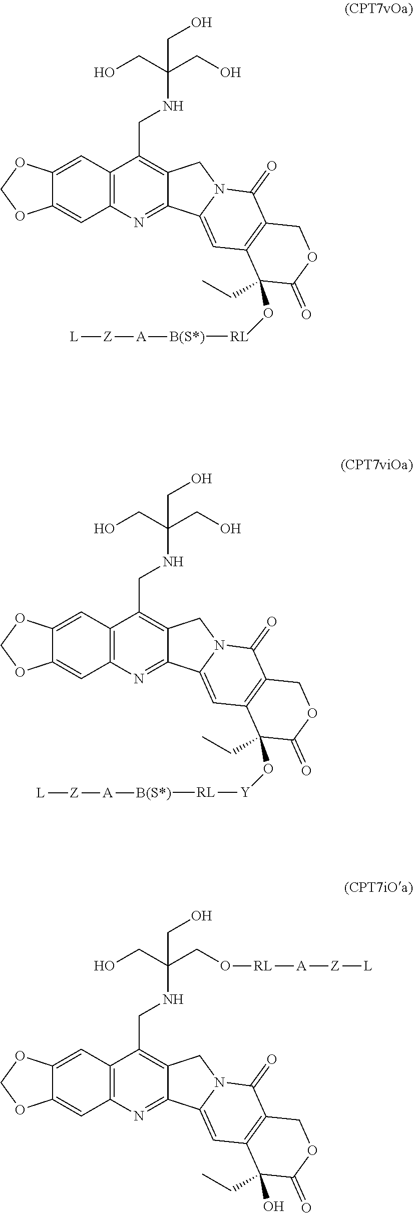

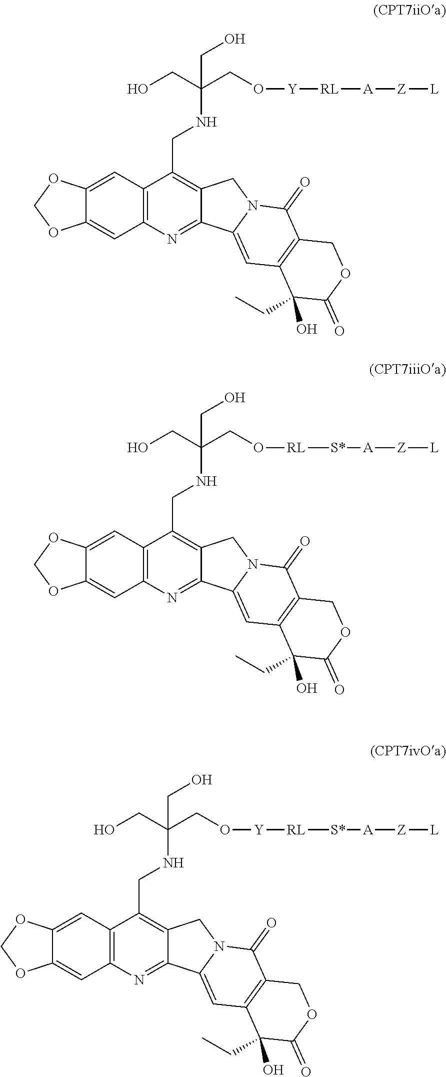

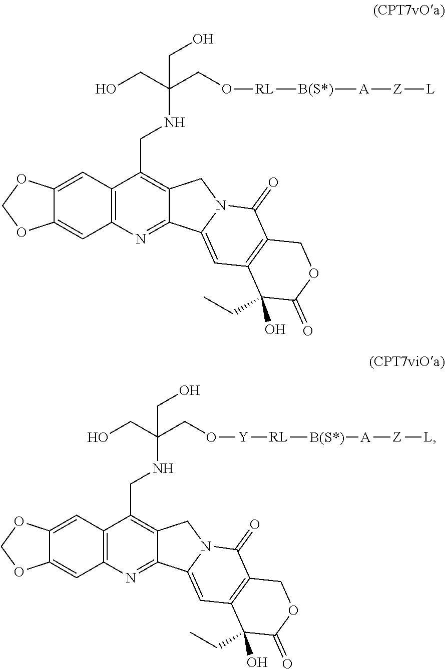

respectively, wherein RL is any one of the Releasable Linkers disclosed herein, preferably RL is a Glucuronide Unit, and the groups L, Z, A, S*, B and Y have the meanings provided above and in any one of the embodiments specifically recited herein.

In other embodiments the ADCs in which Q has the formula of —Z-A-, —Z-A-RL-, —Z-A-S*—W—, —Z-A-B(S*)—W—, —Z-A-S*-RL-, —Z-A-B(S*)—RL-, —Z-A-S*—W-RL- and —Z-A-B(S*)—W-RL- and are comprised of a Drug Unit having formula CPT5 are represented by formulae of:

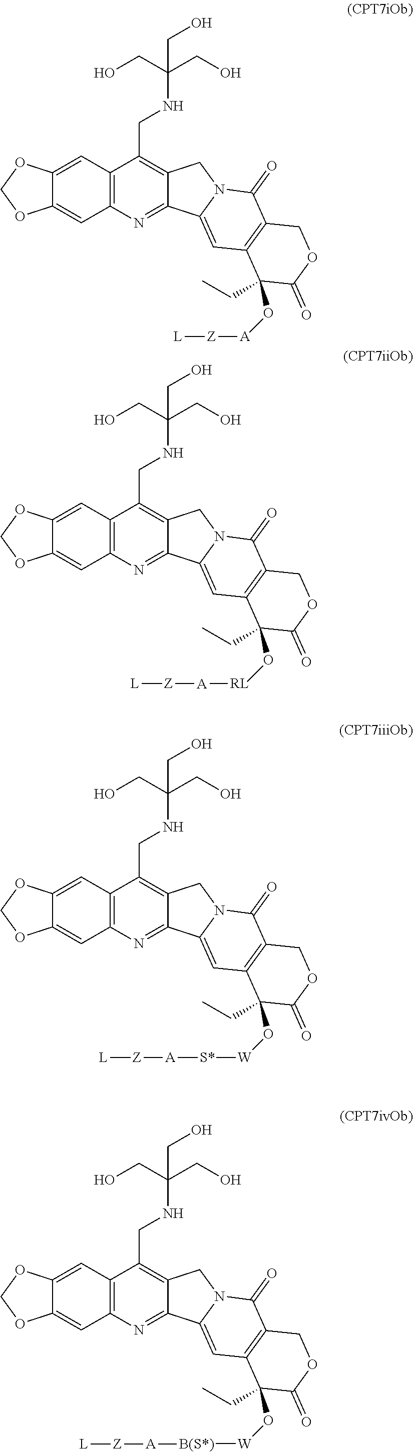

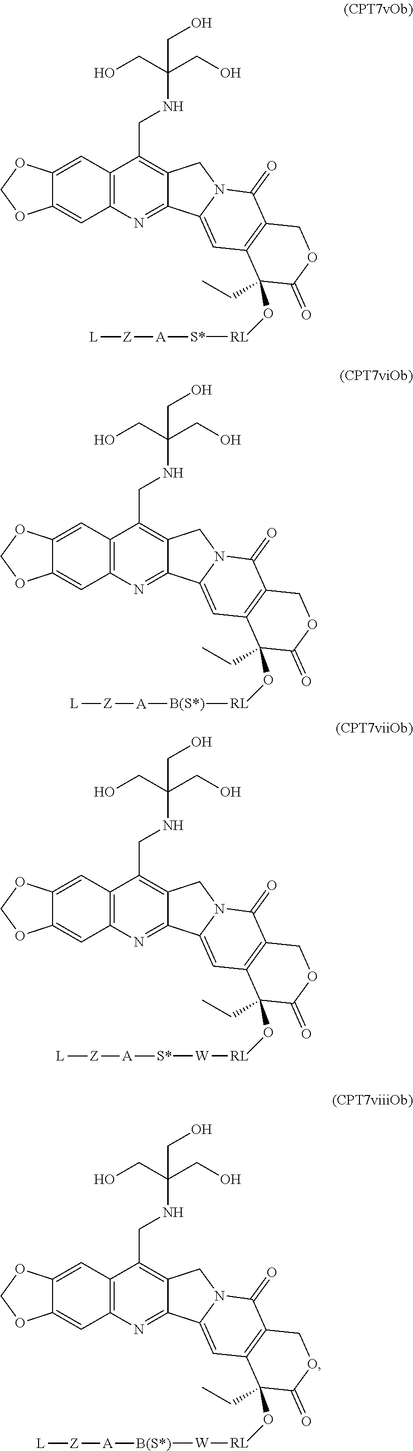

respectively, wherein RL is a Releasable Linker that is other than a Glucuronide Unit and the groups L, Z, A, S*, B and W have the meanings provided above and in any one of the embodiments specifically recited herein.

### 1. Camptothecin-Linker Compounds

In some embodiments, when preparing the ADCs, it will be desirable to synthesize the full drug-linker combination prior to conjugation to a targeting agent (e.g., antibody). In such embodiments, Camptothecin-Linker Compounds as described herein, are intermediate compounds. In those embodiments, the Stretcher Unit in a Camptothecin-Linker compound is not yet covalently attached to the Ligand Unit (i.e., is a Stretcher Unit precursor, Z′), and therefore has a functional group for conjugation to a targeting ligand. In one embodiment, a Camptothecin-Linker compound is comprised of a Camptothecin compound (shown herein as formulae CPT1, CPT2, CPT3, CPT4, CPT5, CPT6 and CPT7), and a Linker Unit (Q) comprising a Glucuronide Unit as a Releasable Linker (RL) through which the Ligand Unit is connected to the Camptothecin.

In another embodiment, a Camptothecin-Linker Compound comprises a Camptothecin compound of formulae CPT1, CPT2, CPT3, CPT4, CPT5, CPT6 or CPT7, and a Linker Unit (Q) comprising a Releasable Linker (RL) that is other than a Glucuronide Unit through which the Ligand Unit is connected to the conjugated Camptothecin compound. Thus, in either embodiment the Linker Unit comprises, in addition to RL, a Stretcher Unit precursor (Z′) comprising a functional group for conjugation to a targeting agent that is the precursor to the Ligand Unit and thus is capable of (directly or indirectly) connecting the RL to the Ligand Unit. In some of those embodiments a Parallel Connector Unit (B) when it is desired to add a Partitioning Agent (S*) as a side chain appendage. In any one of those embodiments, a Connector Unit (A) is present when it is desirable to add more distance between the Stretcher Unit and RL.

In one group of embodiments, a Camptothecin-Linker compound is comprised of a Camptothecin compound having formula CPT1, CPT2, CPT3, CPT4, CPT5, CPT6 or CPT7, and a Linker Unit (Q), wherein Q comprises a Releasable Linker (RL) that is a Glucuronide Unit, directly attached to a Stretcher Unit precursor (Z′) or indirectly to Z′ through attachment to intervening component(s) of the Camptothecin-Linker compound's Linker Unit (i.e., A, S* and/or B(S*)), wherein Z′ is comprised of a functional group capable of forming a covalent bond to a targeting agent.

In another group of embodiments, a Camptothecin-Linker Compound is comprised of a Camptothecin having formula CPT1, CPT2, CPT3, CPT4, CPT5, CPT6 or CPT7, and a Linker Unit (Q), wherein Q comprises a Releasable Linker (RL) that is other than a Glucuronide Unit (RL), directly attached to a Stretcher Unit precursor (Z′) or indirectly to Z′ through attachment to intervening component(s) of the Camptothecin-Linker Compound's Linker Unit (i.e., A, S* and/or B(S*)), wherein Z′ is comprised of a functional group capable of forming a covalent bond to a targeting agent.

In the context of the ADCs and/or the Camptothecin-Linker Compounds—the assembly is best described in terms of its component groups. While some procedures are also described herein, the order of assembly and the general conditions to prepare the Conjugates and Compounds will be well understood by one of skill in the art.

### 2. Component Groups

### Ligand Unit

In some embodiments of the invention, a Ligand Unit is present. The Ligand Unit (L-) is a targeting agent that specifically binds to a target moiety. In one group of embodiments, the Ligand Unit comprises an antibody or antigen binding fragment thereof that binds to CEACAM5. In some embodiments, the Ligand Unit comprises any of the antibodies or antigen binding fragments thereof described herein. The Ligand Unit acts to target and present the camptothecin (e.g., CPT6) to the particular target cell population with which the Ligand Unit interacts due to the presence of its targeted component or molecule (e.g., antibody) and allows for subsequent release of free drug within (i.e., intracellularly) or within the vicinity of the target cells (i.e., extracellularly). Ligand Units, L, include, but are not limited to, proteins, polypeptides and peptides. Suitable Ligand Units include, for example, antibodies, e.g., full-length antibodies and antigen binding fragments thereof, interferons, lymphokines, hormones, growth factors and colony-stimulating factors, vitamins, nutrient-transport molecules (such as, but not limited to, transferrin), or any other cell binding molecule or substance. In some embodiments, the Ligand Unit (L) is from an antibody or a non-antibody protein targeting agent.

In one group of embodiments a Ligand Unit (e.g., an antibody or antigen binding fragment thereof that binds to CEACAM5) is bonded to Q (a Linker Unit) which comprises a Glucuronide Releasable Linker. As noted above, still other linking components can be present in the conjugates described herein to serve the purpose of providing additional space between the Camptothecin drug compound and the Ligand Unit (e.g., a Stretcher Unit and optionally a Connector Unit, A), or providing attributes to the composition to increases solubility (e.g., a Partitioning Agent, S*). In some of those embodiments, the Ligand Unit (e.g., an antibody or antigen binding fragment thereof that binds to CEACAM5) is bonded to Z of the Linker Unit via a heteroatom of the Ligand Unit. Heteroatoms that may be present on a Ligand Unit for that bonding include sulfur (in one embodiment, from a sulfhydryl group of a targeting ligand), oxygen (in one embodiment, from a carboxyl or hydroxyl group of a targeting ligand) and nitrogen, optionally substituted (in one embodiment, from a primary or secondary amine functional group of a targeting ligand or in another embodiment from an optionally substituted amide nitrogen). Those heteroatoms can be present on the targeting ligand in the ligand's natural state, for example in a naturally occurring antibody, or can be introduced into the targeting ligand via chemical modification or biological engineering.

In one embodiment, a targeting agent that is a precursor to a Ligand Unit has a sulfhydryl functional group (such as from a cysteine amino acid) so that the Ligand Unit is bonded to the Linker Unit via the sulfur atom of the sulfhydryl functional group.

In yet another aspect, a targeting agent that is a precursor to Ligand Unit has one or more lysine residues capable of chemical modification to introduce one or more sulfhydryl groups. In those embodiments, the Ligand Unit is covalently attached to the Linker Unit via the sulfhydryl functional group's sulfur atom. The reagents that can be used to modify lysines in that manner include, but are not limited to, N-succinimidyl S-acetylthioacetate (SATA) and 2-Iminothiolane hydrochloride (Traut's Reagent).

In another embodiment, a targeting agent that is a precursor to a Ligand Unit has one or more carbohydrate groups capable of modification to provide one or more sulfhydryl functional groups. The chemically modified Ligand Unit in an ADC is bonded to a Linker Unit component (e.g., a Stretcher Unit) via the sulfur atom of the sulfhydryl functional group.

In yet another embodiment, a targeting agent that is a precursor to a Ligand Unit has one or more carbohydrate groups that can be oxidized to provide an aldehyde (—CHO) functional group (see, e.g., Laguzza, et al., 1989, J. Med. Chem. 32(3):548-55). In these embodiments, the corresponding aldehyde interacts with a reactive site on a Stretcher Unit precursor to form a bond between the Stretcher Unit and the Ligand Unit. Reactive sites on a Stretcher Unit precursor that capable of interacting with a reactive carbonyl-containing functional group on a targeting Ligand Unit include, but are not limited to, hydrazine and hydroxylamine. Other protocols for the modification of proteins for the attachment of Linker Units (Q) or related species are described in Coligan et al., Current Protocols in Protein Science, vol. 2, John Wiley & Sons (2002) (incorporated herein by reference).

In some aspects, a targeting agent that is a precursor to a Ligand Unit t is capable of forming a bond by interacting with a reactive functional group on a Stretcher Unit precursor (Z′) to form a covalent bond between the Stretcher Unit (Z) and the Ligand Unit, which corresponds in structure to the targeting agent. The functional group of Z′ having that capability for interacting with a targeting agent will depend on the nature of the targeting agent that will correspond in structure to the Ligand Unit. In some embodiments, the reactive group is a maleimide that is present on a Stretcher Unit prior to its attachment to form a Ligand Unit (i.e., a maleimide moiety of a Stretcher Unit precursor). Covalent attachment of a Ligand Unit to a Stretcher Unit is accomplished through a sulfhydryl functional group of a targeting agent that is a precursor to a Ligand Unit interacting with the maleimide functional group of Z′ to form a thio-substituted succinimide. The sulfhydryl functional group can be present on the targeting agent in the targeting agent's natural state, for example, in a naturally occurring residue, or can be introduced into the targeting agent via chemical modification or by biological engineering.

In still another embodiment, the Ligand Unit is from an antibody that binds to CEACAM5 and the sulfhydryl group is generated by reduction of an interchain disulfide of the antibody. Accordingly, in some embodiments, the Linker Unit is conjugated to a cysteine residue from reduced interchain disulfide(s).

In yet another embodiment, the Ligand Unit is from an antibody and the sulfhydryl functional group is chemically introduced into the antibody, for example, by introduction of a cysteine residue. Accordingly, in some embodiments, the Linker Unit (with or without an attached Camptothecin) is conjugated to a Ligand Unit through an introduced cysteine residue of a Ligand Unit.

It has been observed for bioconjugates that the site of drug conjugation can affect a number of parameters including ease of conjugation, drug-linker stability, effects on biophysical properties of the resulting bioconjugates, and in vitro cytotoxicity. With respect to drug-linker stability, the site of conjugation of a drug-linker moiety to a Ligand Unit can affect the ability of the conjugated drug-linker moiety to undergo an elimination reaction, in some instances, to cause premature release of free drug. Sites for conjugation on a targeting agent include, for example, a reduced interchain disulfide as well as selected cysteine residues at engineered sites. In some embodiments conjugation methods to form ADCs as described herein use thiol residues at genetically engineered sites that are less susceptible to the elimination reaction (e.g., positions 239 according to the EU index as set forth in Kabat) in comparison to conjugation methods that use thiol residues from a reduced disulfide bond. In other embodiments conjugation methods to form ADCs as described herein use thiol residues resulting from interchain disulfide bond reduction. In some embodiments, the Ligand Unit is from an antibody that binds to CEACAM5.

### Camptothecin Compounds

The Camptothecin compounds utilized in the various embodiments described herein are represented by the formulae:

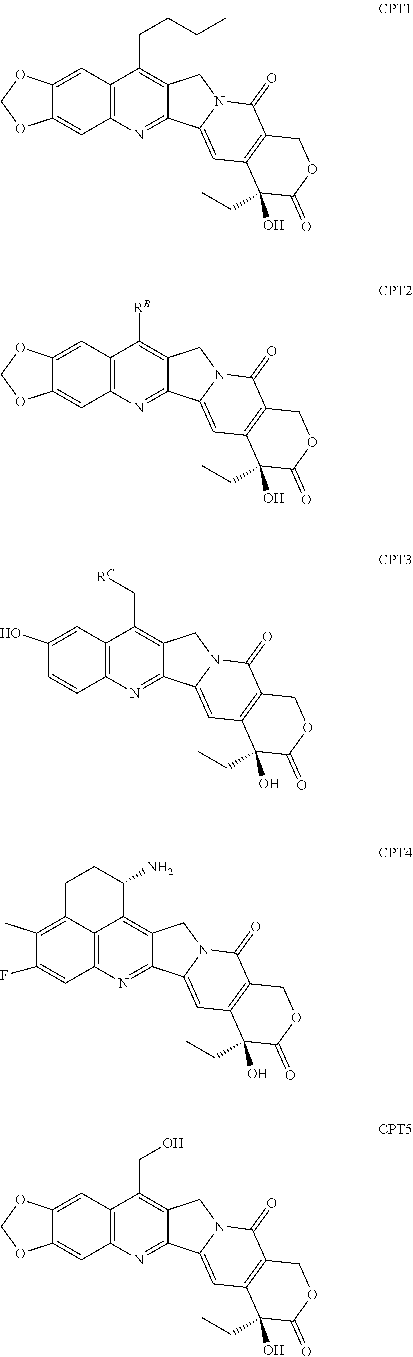

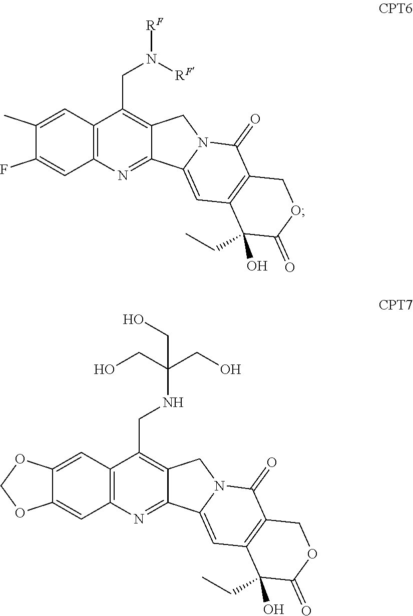

wherein R^{B} is a moiety selected from the group consisting of —H, C₁-C₈ alkyl, C₁-C₈ haloalkyl, C₃-C₈ cycloalkyl, (C₃-C₈ cycloalkyl)-C₁-C₄ alkyl-, phenyl and phenyl-C₁-C₄ alkyl-;

R^{C} is a moiety selected from the group consisting of C₁-C₆ alkyl and C₃-C₆ cycloalkyl;

each R^{F} and R^{F′} is a moiety independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl, N—(C₁-C₄ hydroxyalkyl)-C₁-C₈ aminoalkyl-, C₁-C₈ alkylC(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl C₁-C₄ alkyl, heteroaryl and heteroaryl-C₁-C₄ alkyl, or

R_{F} and R^{F′} are combined with the nitrogen atom to which both are attached to form a 5-, 6- or 7-membered ring having 0 to 3 substituents independently selected from the group consisting of halogen, C₁-C₄ alkyl, —OH, —OC₁-C₄ alkyl, —NH₂, —NHC₁-C₄ alkyl and N(C₁-C₄ alkyl)₂,

wherein the cycloalkyl, heterocycloalkyl, phenyl and heteroaryl moieties of R^{B}, R^{C}, R^{F} and R^{F′} are substituted with from 0 to 3 substituents independently selected from the group consisting of halogen, C₁-C₄ alkyl, —OH, —OC₁-C₄ alkyl, —NH₂, —NHC₁-C₄ alkyl and —N(C₁-C₄ alkyl)₂.

Still other Camptothecin compounds useful in the context of the ADCs and Camptothecin Linker compounds described herein are Camptothecin compounds 14a-14z of Table I and compound 18a-18r of Table J, and Camptothecin compounds that have a five- or six-ring fused framework analogs to those structures provided as formulae CPT1, CPT2, CPT3, CPT4, CPT5, CPT6, CPT7, 14a-14z and 18a-18r, which in some embodiments have an additional group including, but not limited to a hydroxyl, thiol, amine or amide functional group whose oxygen, sulfur or optionally substituted nitrogen atom is capable of incorporation into a linker, and is capable of being released from an ADC as a free drug. In some embodiments, that functional group provides the only site on the camptothecin compound available for attachment to the Linker Unit (Q). The resulting drug-linker moiety of an ADC is one that is capable of releasing active free drug at the site targeted by its Ligand Unit in order to exert a cytotoxic, cytostatic or immunosuppressive effect.

“Free drug” refers to drug, as it exists once released from the drug-linker moiety. In some embodiments, the free drug includes a fragment of the Releasable Linker or Spacer Unit (Y) group. Free drug, which includes a fragment of the Releasable Linker or Spacer Unit (Y), are released from the remainder of the drug-linker moiety via cleavage of the releasable linker or released via the cleavage of a bond in the Spacer Unit (Y) group and is biologically active after release. In some embodiments, the free drug differs from the conjugated drug in that the functional group of the free drug for attachment to the self-immolative assembly unit is no longer associated with components of the ADC (other than a previously shared heteroatom). For example, the free hydroxyl functional group of an alcohol-containing drug can be represented as D-O*H, whereas in the conjugated form the oxygen heteroatom designated by O* is incorporated into the methylene carbamate unit of a self-immolative unit. Upon activation of the self-immolative moiety and release of free drug, the covalent bond to O* is replaced by a hydrogen atom so that the oxygen heteroatom designated by O* is present on the free drug as —O—H.

### Linker Unit (Q)

As noted above, is some embodiments, the Linker Unit Q has a formula selected from the group consisting of:

Z-A-RL-;—Z-A-RL-Y—;—Z-A-S*-RL-;—Z-A-B(S*)—RL-; —Z-A-S*-RL-Y—; and —Z-A-B(S*)—RL-Y—;

wherein Z is a Stretcher Unit; A is a bond or a Connector Unit; B is a Branching Unit; S* is a Partitioning Agent; RL is Releasable Linker that is a Glucuronide Unit; and Y is a Spacer Unit; and

wherein the point of attachment of D to Q is through any one of the heteroatoms of the hydroxyl and primary and secondary amines present on CPT1, CPT2, CPT3, CPT4, CPT5, CPT6 or CPT7 or any one of compounds 14a-14z of Table I and compounds 18a-18r of Table J.

In other embodiments, the Linker Unit Q has a formula selected from the group consisting of:

—Z-A-;—Z-A-RL-;—Z-A-S*—W—;—Z-A-B(S*)—W—;—Z-A-S*-RL-;—Z-A-B(S*)—RL-; —Z-A-S*—W-RL-; and —Z-A-B(S*)—W-RL-;

wherein Z is a Stretcher Unit, A is a bond or a Connector Unit; B is a Parallel Connector Unit; S* is a Partitioning Agent; RL is a Releasable Linker other than a Glucuronide Unit; and W is an Amino Acid Unit; and

wherein the point of attachment to Q is through the hydroxyl group substituent of the lactone ring of CPT1, CPT2, CPT3, CPT4, CPT5, CPT6 or CPT7 or any one of compounds 14a-14z of Table I and compounds 18a-18r of Table J.

In one group of embodiments, Q has a formula selected from the group consisting of: —Z-A-S*-RL- and —Z-A-S*-RL-Y—.

In another group of embodiments, Q has a formula selected from the group consisting of —Z-A-B(S*)—RL- and —Z-A-B(S*)—RL-Y—.

In still another group of embodiments, Q has a formula selected from the group consisting of —Z-A-RL- and —Z-A-RL-Y—.

### Stretcher Unit (Z) or (Z′)

A Stretcher Unit (Z) is a component of an ADC or a Camptothecin-Linker Compound or other Intermediate that acts to connect the Ligand Unit to the remainder of the conjugate. In that regard a Stretcher Unit, prior to attachment to a Ligand Unit (i.e. a Stretcher Unit precursor, Z′), has a functional group that can form a bond with a functional group of a targeting ligand (e.g., antibody).

In some embodiments, a Stretcher Unit precursor (Z′) has an electrophilic group that is capable of interacting with a reactive nucleophillic group present on a Ligand Unit (e.g., an antibody) to provide a covalent bond between a Ligand Unit and the Stretcher Unit of a Linker Unit. Nucleophillic groups on an antibody having that capability include but are not limited to, sulfhydryl, hydroxyl and amino functional groups. The heteroatom of the nucleophillic group of an antibody is reactive to an electrophilic group on a Stretcher Unit precursor and provides a covalent bond between the Ligand Unit and Stretcher Unit of a Linker Unit or Drug-Linker moiety. Useful electrophilic groups for that purpose include, but are not limited to, maleimide, haloacetamide groups, and NHS esters. The electrophilic group provides a convenient site for antibody attachment to form an ADC or Ligand Unit-Linker intermediate.

In other embodiments, a Stretcher Unit precursor has a reactive site which has a nucleophillic group that is reactive to an electrophilic group present on a Ligand Unit (e.g., an antibody). Useful electrophilic groups on an antibody for that purpose include, but are not limited to, aldehyde and ketone carbonyl groups. The heteroatom of a nucleophillic group of a Stretcher Unit precursor can react with an electrophilic group on an antibody and form a covalent bond to the antibody. Useful nucleophillic groups on a Stretcher Unit precursor for that purpose include, but are not limited to, hydrazide, hydroxylamine, amino, hydrazine, thiosemicarbazone, hydrazine carboxylate, and arylhydrazide. The electrophilic group on an antibody provides a convenient site for antibody attachment to form an ADC or Ligand Unit-Linker intermediate.

In some embodiments, a sulfur atom of a Ligand Unit is bound to a succinimide ring system of a Stretcher Unit formed by reaction of a thiol functional group of a targeting ligand with a maleimide moiety of the corresponding Stretcher Unit precursor. In other embodiments, a thiol functional group of a Ligand Unit reacts with an alpha haloacetamide moiety to provide a sulfur-bonded Stretcher Unit by nucleophillic displacement of its halogen substituent.

Representative Stretcher Units of such embodiments include those having the structures of:

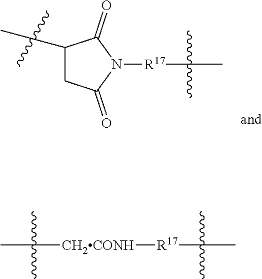

wherein the wavy line adjacent to R¹⁷ indicates attachment to the Parallel Connector Unit (B) or Connector Unit (A) if B is absent, or a Partitioning Agent (S*), if B is absent, the other wavy line indicates covalent attachment to a sulfur atom of a Ligand Unit and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In some embodiments, the R¹⁷ group is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein subscript x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

An illustrative Stretcher Unit is that of Formula Za or Za-BU in which R¹⁷ is —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, or —(C₃-C₈ heterocyclo)-C₁-C₁₀ alkylene-C(═O)—.

Accordingly, some preferred embodiments are represented by formula Za and Za-BU:

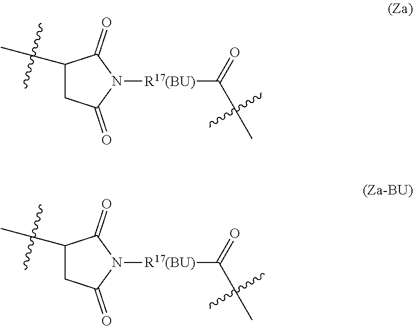

wherein the wavy line adjacent the carbonyl carbon atom indicates attachment to B, A, or S*, in the formulae above, depending on the presence or absence of A and/or B, and the other wavy line indicates covalent bonding of the succinimide ring carbon atom to a sulfur atom of a Ligand Unit. During synthesis, the basic amino functional group of the Basic Unit (BU) can be protected by a protecting group.

More preferred embodiments of Stretcher Units of formula Za and Za-BU are as follows:

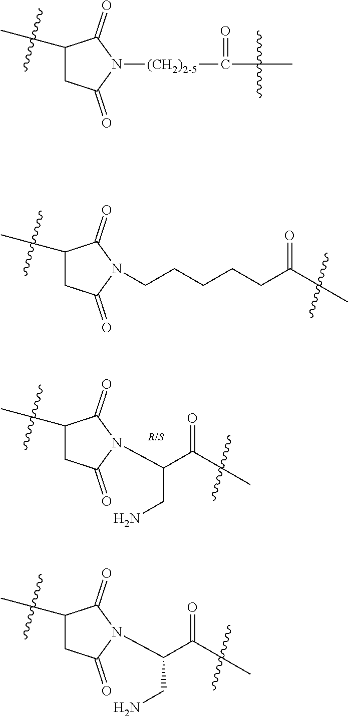

wherein the wavy line adjacent the carbonyl carbon atom indicates attachment to B, A, or S*, in the formulae above, depending on the presence or absence of A and/or B, and the other wavy line indicates covalent bonding of the succinimide ring carbon atom to a sulfur atom of a Ligand Unit.

It will be understood that a Ligand Unit-substituted succinimide may exist in hydrolyzed form(s). Those forms are exemplified below for hydrolysis of Za or Za-BU, wherein the structures representing the regioisomers from that hydrolysis have formula Zb and Zc or Zb-BU and Zc-BU.

Accordingly, in other preferred embodiments a Stretcher unit (Z) is comprised of a succinic acid-amide moiety represented by the following:

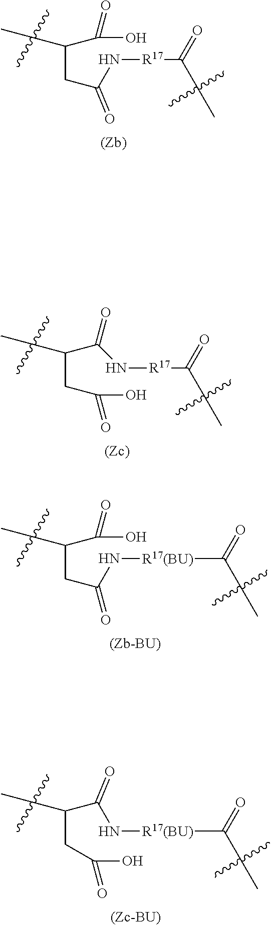

wherein the wavy line adjacent to the carbonyl carbon atom bonded to R¹⁷ and the wavy line adjacent to the carbon atom of the acid-amide moiety is as defined for Za or Za-BU, depending on the presence or absence of A and/or B; and R¹⁷ is —C₁-C₈ alkylene-, wherein in Zb-BU and Zc-BU the alkylene is substituted by a Basic Unit (BU), wherein BU is —(CH₂)ₓNH₂, —(CH₂)ₓNHR^{a}, or —(CH₂)ₓN(R^{a})₂, wherein subscript x is an integer of from 1-4 and each R^{a} is independently selected from the group consisting of C₁₋₆ alkyl and C₁₋₆ haloalkyl, or both R^{a} together with the nitrogen to which they are attached define an azetidinyl, pyrrolidinyl or piperidinyl group.

In more preferred embodiment, —Z-A- comprises a moiety derived from a maleimido-alkanoic acid moiety or an mDPR moiety. See, for example, see WO 2013/173337. In one group of embodiments, Z-A- is derived from a maleimido-propionyl moiety.

Accordingly in some of those more preferred embodiments, a Stretcher unit (Z) is comprised of an succinic acid-amide moiety represented by the structure of formula Zb′, Zc′, (R/S)-Zb′—BU, (S)-Zb′—BU, (R/S)-Zc′—BU or (S)-Zc′—BU as follows:

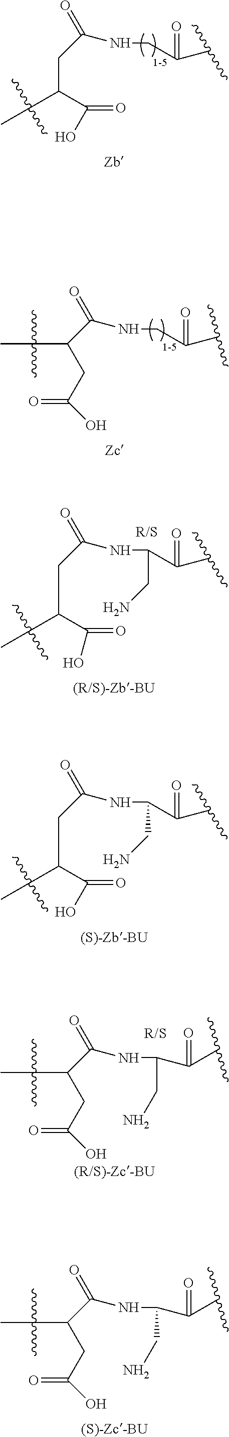

wherein the wavy lines are as defined for Za or Za-BU.

In particularly preferred embodiments a Stretcher unit (Z) is comprised of a succinimide moiety represented by the structure of

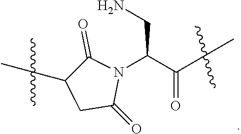 [SMILES: *C(=O)[C@H](CN)N1C(=O)CC(*)C1=O]

or is comprised of a succinic acid-amide moiety represented by the structure of:

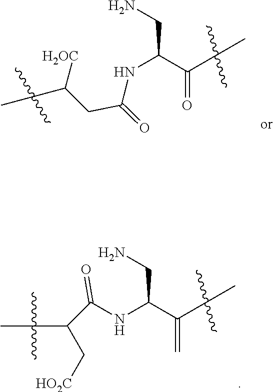

Illustrative Stretcher Units bonded to a Connector Unit (A) which are comprised of Za′, Zb′ or Zc′, in which —R¹⁷— of Za, Zb or Zc is —CH₂— or —CH₂CH₂—, or are comprised of Za′—BU, Zb′—BU or Zc′—BU in which —R¹⁷(BU)— of Za-BU, Zb-BU or Zc-BU is —CH(CH₂NH₂)—, have the following structures:

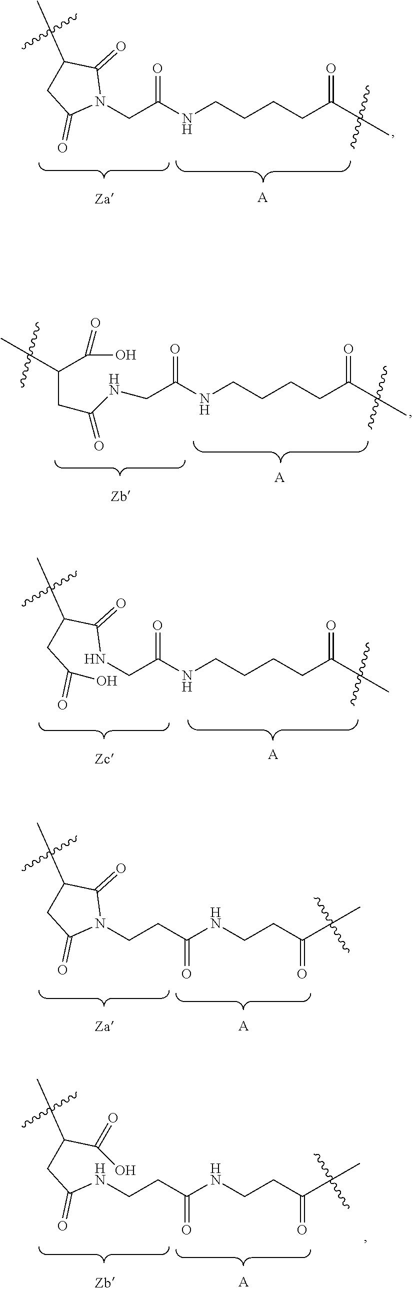

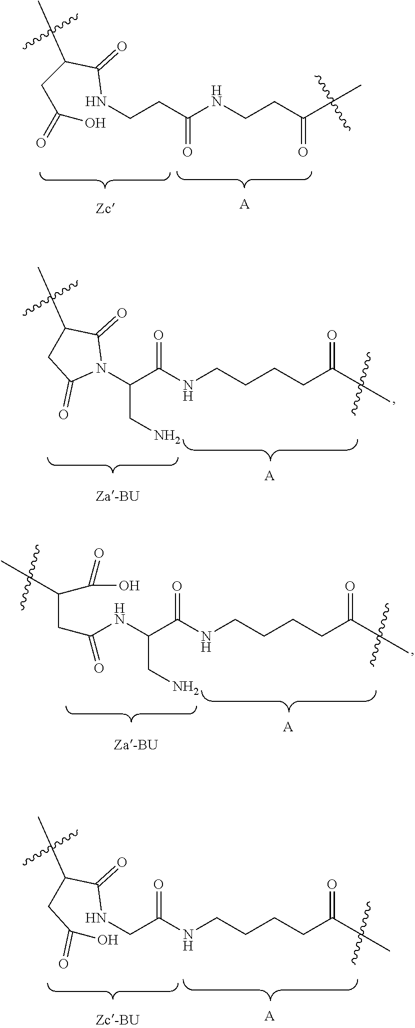

wherein the wavy lines are as defined for Za or Za-BU.

Other Stretcher Units bonded to a Ligand Unit (L) and a Connector Unit (A) have the structures above wherein A in any one of the above -Za-A-, -Za(BU)-A-, -Za′-A-, -Za′(BU)-A-, -Zb-A-, -Zb(BU)-A-, -Zb′-A-, -Zb′(BU)—, -Zc′-A- and Zc′(BU)-A- structures is replaced by a Parallel Connector Unit having the structure of:

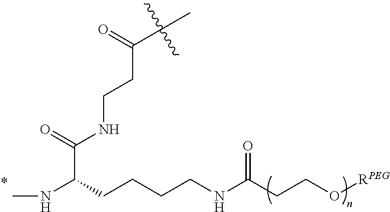

wherein subscript n ranges from 8 to 24; R^{PEG} is a PEG Unit capping group, preferably —CH₃ or —CH₂CH₂CO₂H, the asterisk (*) indicates covalent attachment to a Stretcher Unit corresponding in structure to formula Za, Za′, Zb′ or Zc′ and the wavy line indicates covalent attachment to the Releasable Linker (RL).

Illustrative Stretcher Units prior to conjugation to the Ligand Unit (i.e., Stretcher Unit precursors) are comprised of a maleimide moiety and are represented by structures including that of formula Z′a

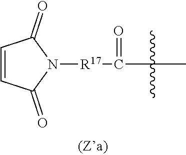

wherein the wavy line adjacent the carbonyl carbon atom indicates attachment to B, A, or S*, in the formulae above, depending on the presence or absence of A and/or B, R¹⁷ is —(CH₂)₁₋₅—, optionally substituted with a Basic Unit, such as an optionally substituted aminoalkyl, e.g., —(CH₂)ₓNH₂, —(CH₂)ₓNHR^{a}, and —(CH₂)ₓN(R^{a})₂, wherein subscript x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

Other illustrative Stretcher Units prior to conjugation to the Ligand Unit (i.e., Stretcher Unit precursors) are comprised of a maleimide moiety and are represented by structures including that of formula Z′a-BU.

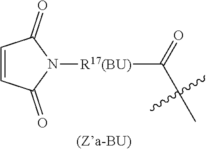

wherein the wavy line adjacent the carbonyl carbon atom indicates attachment to B, A, or S*, in the formulae above, depending on the presence or absence of A and/or B, R¹⁷ is —(CH₂)₁₋₅—, substituted with a Basic Unit, such as an optionally substituted aminoalkyl, e.g., —(CH₂)ₓNH₂, —(CH₂)ₓNHR^{a}, and —(CH₂)ₓN(R^{a})₂, wherein subscript x is an integer of from 1-4, preferably R¹⁷ is —CH₂— or —CH₂CH₂— and subscript x is 1 or 2, and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

In some preferred embodiments of formula Z′a, a Stretcher Unit precursor is represented by one of the following structures:

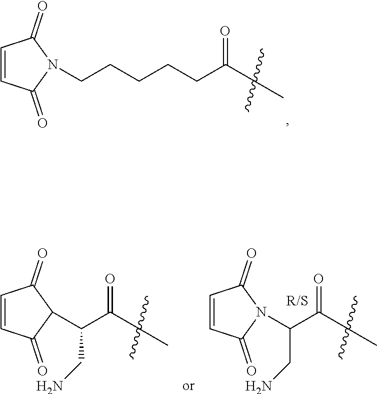 [SMILES: *C(=O)CCCCCN1C(=O)C=CC1=O ; *C(=O)[C@H](CN)C1C(=O)C=CC1=O ; *C(=O)C(CN)N1C(=O)C=CC1=O]

wherein the wavy line adjacent to the carbonyl is as defined for Z′a or Z′a-BU.

In more preferred embodiments the Stretcher unit precursor (Z′) is comprised of a maleimide moiety and is represented by the structure of:

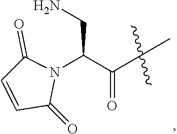 [SMILES: *C(=O)[C@H](CN)N1C(=O)C=CC1=O]

wherein the wavy line adjacent to the carbonyl is as defined for Za′ and the amino group is optional protonated or protected by an amino protecting group.

In Stretcher Units having a BU moiety, it will be understood that the amino functional group of that moiety is typically protected by an amino protecting group during synthesis, e.g., an acid labile protecting group (e.g., BOC).

Illustrative Stretcher Unit precursors covalently attached to a Connector Unit that are comprised of the structure of Z′a or Z′a-BU in which —R¹⁷— or —R¹⁷(BU)— is —CH₂—, —CH₂CH₂— or —CH(CH₂NH₂)— have the following structures:

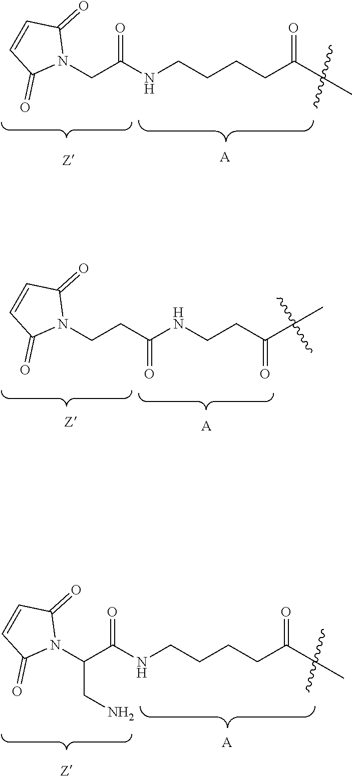

wherein the wavy line adjacent to the carbonyl is as defined for Z′a or Z′a-BU.

Other Stretcher Unit precursors bonded a Connector Unit (A) have the structures above wherein A in any one of the above Z′-A- and Z′(BU)-A- structures is replaced by a Parallel Connector Unit and Partitioning Agent (—B(S*)—) having the structure of

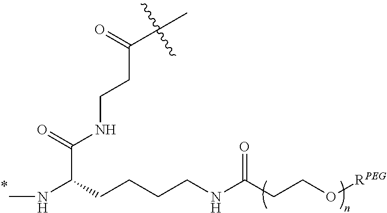

wherein subscript n ranges from 8 to 24; R^{PEG} is a PEG Unit capping group, preferably-CH₃ or —CH₂CH₂CO₂H, the asterisk (*) indicates covalent attachment to the Stretcher Unit precursor corresponding in structure to formula Za or Za′ and the wavy line indicates covalent attachment to RL. In instances such as those shown here, the shown PEG group is meant to be exemplary of a variety of Partitioning Agents including PEG groups of different lengths and other Partitioning Agents that can be directly attached or modified for attachment to the Parallel Connector Unit.

In another embodiment, the Stretcher Unit is attached to the Ligand Unit via a disulfide bond between a sulfur atom of the Ligand Unit and a sulfur atom of the Stretcher unit. A representative Stretcher Unit of this embodiment is depicted within the square brackets of Formula Zb:

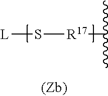

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In yet another embodiment, the reactive group of a Stretcher Unit precursor contains a reactive site that can form a bond with a primary or secondary amino group of a Ligand Unit (e.g., antibody). Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher Units of this embodiment are depicted within the square brackets of Formulas Zci, Zcii and Zciii:

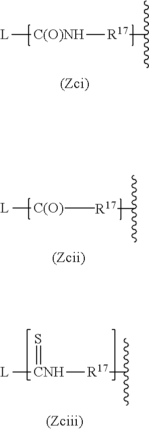

wherein the wavy line indicates attachment to the Parallel Connector Unit (B) or Connector Unit (A) if B is absent or a Partitioning Agent (S*), if A and B are absent and R¹⁷ is —C₁-C₁₀ alkylene-, —C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In still other embodiments, the reactive group of the Stretcher Unit precursor contains a reactive nucleophile that is capable of reacting with an electrophile present on, or introduced to, a Ligand Unit. For example, a carbohydrate moiety on a targeting ligand can be mildly oxidized using a reagent such as sodium periodate and the resulting electrophilic functional group (—CHO) of the oxidized carbohydrate can be condensed with a Stretcher Unit precursor that contains a reactive nucleophile such as a hydrazide, an oxime, a primary or secondary amine, a hydrazine, a thiosemicarbazone, a hydrazine carboxylate, or an arylhydrazide such as those described by Kaneko, T. et al. (1991) Bioconjugate Chem. 2:133-41. Representative Stretcher Units of this embodiment are depicted within the square brackets of Formulas Zdi, Zdii, and Zdiii:

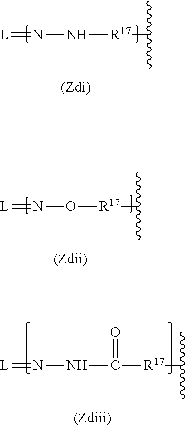

wherein the wavy line indicates attachment to the Parallel Connector Unit (B) or Connector Unit (A), or a Partitioning Agent (S*), if A and B are absent and R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

In some aspects of the prevent invention the Stretcher Unit has a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

### Connector Unit (A)

In some embodiments, a Connector Unit (A), is included in an ADC or Camptothecin-Linker Compound in instances where it is desirable to add additional distance between the Stretcher Unit (Z) or precursor thereof (Z′) and the Releasable Linker. In some embodiments, the extra distance will aid with activation within RL. Accordingly, the Connector Unit (A), when present, extends the framework of the Linker Unit. In that regard, a Connector Unit (A) is covalently bonded with the Stretcher Unit (or its precursor) at one terminus and is covalently bonded to the optional Parallel Connector Unit or the Partitioning Agent (S*) at its other terminus.

The skilled artisan will appreciate that the Connector Unit can be any group that serves to provide for attachment of the Releasable Linker to the remainder of the Linker Unit (Q). The Connector Unit can be, for example, comprised of one or more (e.g., 1-10, preferably, 1, 2, 3, or 4) natural or non-natural amino acid, amino alcohol, amino aldehyde, diamino residues. In some embodiments, the Connector Unit is a single natural or non-natural amino acid, amino alcohol, amino aldehyde, or diamino residue. An exemplary amino acid capable of acting as Connector units is β-alanine.

In some of those embodiments, the Connector Unit has the formula denoted below:

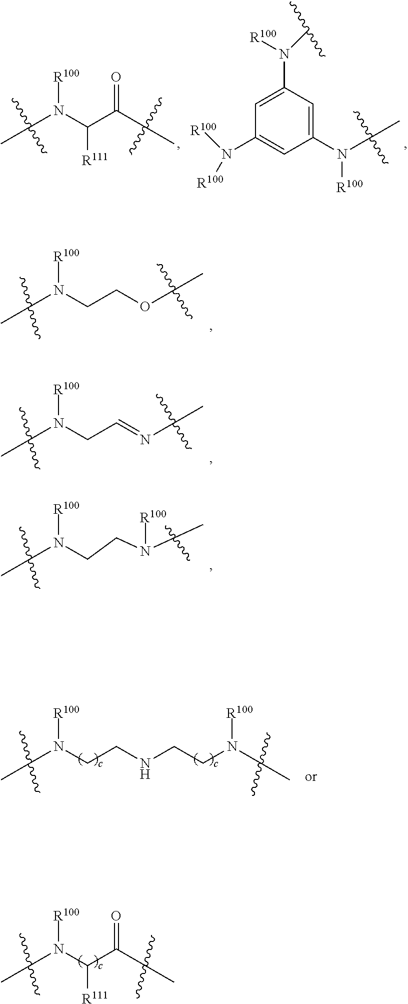

wherein the wavy lines indicate attachment of the Connector Unit within the ADC or Camptothecin Linker Compound; and wherein R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-2OH—CH₂, and SCH

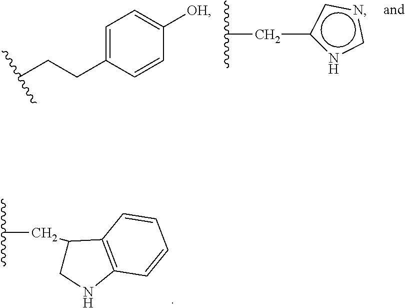

and each R¹⁰⁰ is independently selected from hydrogen or —C₁-C₃ alkyl, preferably hydrogen or CH₃; and subscript c is an independently selected integer from 1 to 10, preferably 1 to 3.

A representative Connector Unit having a carbonyl group for attachment to the Partitioning Agent (S*) or to —B(S*)— is as follows:

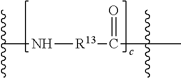

wherein in each instance R¹³ is independently selected from the group consisting of —C₁-C₆ alkylene-, —C₃-C₈carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and the subscript c is an integer ranging from 1 to 4. In some embodiments R¹³ is —C₁-C₆ alkylene and c is 1.

Another representative Connector Unit having a carbonyl group for attachment to Partitioning Agent (S*) or to —B(S*)— is as follows:

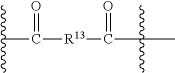

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, or —(C₃-C₈ heterocyclo)-C₁-C₁₀ alkylene-. In some embodiments R¹³ is —C₁-C₆ alkylene.

A representative Connector Unit having a NH moiety that attaches to Partitioning Agent (S*) or to —B(S*)— is as follows:

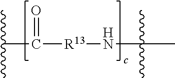

wherein in each instance, R¹³ is independently selected from the group consisting of —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, and subscript c is from 1 to 14. In some embodiments R¹³ is —C₁-C₆ alkylene and subscript c is 1.

Another representative Connector Unit having a NH moiety that attaches to Partitioning Agent (S*) or to —B(S*)— is as follows:

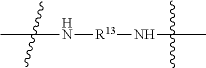

wherein R¹³ is —C₁-C₆ alkylene-, —C₃-C₈carbocyclo-, -arylene-, —C₁-C₁₀ heteroalkylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, —(C₃-C₈ heterocyclo)-C₁-C₁₀ alkylene-, —C(═O)C₁-C₆ alkylene- or —C₁-C₆ alkylene-C(═O)—C₁-C₆ alkylene.

Selected embodiments of Connector Units include those having the following structure of:

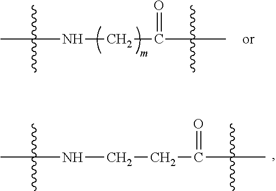

wherein the wavy line adjacent to the nitrogen indicates covalent attachment a Stretcher Unit (Z) (or its precursor Z′), and the wavy line adjacent to the carbonyl indicates covalent attachment to Partitioning Agent (S*) or to —B(S*)—; and m is an integer ranging from 1 to 6, preferably 2 to 6, more preferably 2 to 4.

In some embodiments, unless otherwise noted, “connector” and “connecter” are used interchangeably.

### Releasable Linker (RL)

A Glucuronide Unit is one type of Releasable Linker that provides a mechanism for separation of the Camptothecin from the Ligand Unit and other components of the Linker Unit through activation of a self-immolation cascade within the Linker Unit. In such embodiments, a self-immolation cascade is activated by operation of a glycosidase on a carbohydrate moiety of the Glucuronide Unit. A number of sugars or sugar moieties are useful in the embodiments described herein. Particular carbohydrate moieties (e.g., sugar moieties) include those of Galactose, Glucose, Mannose, Xylose, Arabinose, Mannose-6-phosphate, Fucose, Rhamnose, Gulose, Allose, 6-deoxy-glucose, Lactose, Maltose, Cellobiose, Gentiobiose, Maltotriose, GlcNAc, GalNAc and maltohexaose.

A glycoside unit typically comprises a sugar moiety (Su) linked via an oxygen glycosidic bond to a self-immolative spacer. Cleavage of the oxygen glycosidic bond initiates the self-immolation reaction sequence that result in release of free drug. In some embodiments, the self-immolation sequence is activated from cleavage by β-glucuronidase of a Glucuronide Unit, which is an exemplary glycoside unit. The Glucuronide unit comprises an activation unit and a self-immolative Spacer Unit. The Glucuronide unit comprises a sugar moiety (Su) linked via an oxygen glycosidic bond to a self-immolative Spacer Unit.

In some embodiments, a Glucuronide Unit comprises a sugar moiety (Su) linked via an oxygen glycoside bond (—O′—) to a Self-immolative Unit (SP) of the formula:

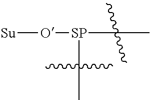

wherein the wavy lines indicate covalent attachment to the Drug Unit of any one of formulae CPT1, CPT2, CPT3, CPT4, CPT5 CPT6 and CPT7, or to a Spacer Unit that is attached to the Drug Unit (a Camptothecin Compound), and to the Stretcher Unit (Z) or its precursor (Z′), either directly or indirectly through the Connector Unit (A) or Parallel Connector Unit (B), Partitioning Agent (S*) or combinations of the Connector Unit and Parallel Connector Unit, as the case may be.

The oxygen glycosidic bond (—O′—) is typically a β-glucuronidase-cleavage site (i.e., Su is from glucuronide), such as a glycoside bond cleavable by human, lysosomal β-glucuronidase.

In some embodiments, the Glucuronide Unit can be represented by formula Ga or Gb:

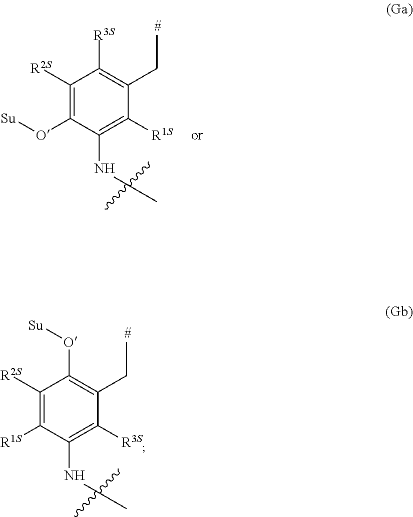

wherein Su is a Sugar moiety, —O′— represents an oxygen glycosidic bond; R^{1S}, R^{2S} and R^{3S} independently are hydrogen, a halogen, —CN, —NO₂, or other electron withdrawing group, or an electron donating group; and wherein the wavy line indicates attachment to a Stretcher Unit (Z) (or its precursor (Z′), either directly or indirectly through a Connector Unit or Parallel Connector Unit or Connector unit and Parallel Connector Unit); and # indicates attachment to the Camptothecin or to a Spacer (either directly or indirectly via an intervening functional group or other moiety).

In preferred embodiments R^{1S}, R^{2S} and R^{3S} are independently selected from hydrogen, halogen, —CN, or —NO₂. In other preferred embodiments, R^{1S}, R^{2S} and R^{3S} are each hydrogen. In other preferred embodiments R^{2S} is an electron withdrawing group, preferably NO₂, and R_{1S} and R^{3S} are each hydrogen.

In some such aspects the activatable self-immolative group capable of glycosidase cleavage to initiate the self-immolative reaction sequence is represented by the formula Gc:

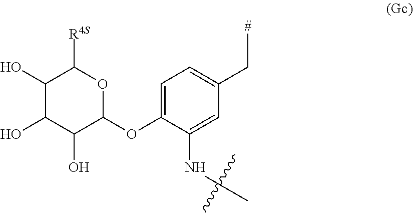

wherein R^{4S} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to the methylene carbamate unit.

In some embodiments wherein the activatable self-immolative moiety is comprised of a Glucuronide Unit, it is represented by the following formula Gd:

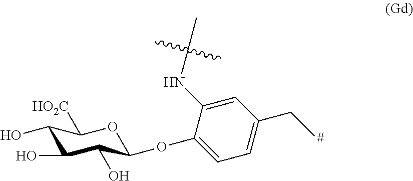

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark (#) indicates covalent attachment of the benzylic carbon of a Spacer or functional group attached to the Camptothecin.

Another type of Releasable Linker that provides a mechanism for separation of the Camptothecin from the Ligand Unit and other components of the Linker Unit through activation of a self-immolation cascade within the Linker Unit is comprised of a p-aminobenzyloxycarbonyl (PAB) moiety whose phenylene component is substituted with Jₘ wherein the subscript m indicating the number of substituents is an integer ranging from 0-4, and each J is independently —C₁-C₈ alkyl, —O—(C₁-C₈ alkyl), -halogen, -nitro or -cyano.

In some embodiments, RL is a self-immolative group capable of releasing -D without the need for a separate hydrolysis step or subsequent self-immolative event. In some embodiments, —RL- is a PAB moiety that is linked to the carbonyl of —W— via the amino nitrogen atom of the PAB group, and connected directly to -D via a carbonate group. In related embodiments, —RL- is comprised of a PAB moiety that is linked to the carbonyl of -A-, —S*— or —B— via the amino nitrogen atom of the PAB group, and connected directly to -D via a carbonate group. Without being bound by any particular theory or mechanism, a possible mechanism of Drug release from RL comprised of a PAB moiety in which RL is attached directly to -D via a carbonate group is shown in Toki et al. (2002) J Org. Chem. 67:1866-1872.

In some embodiments, RL units containing a PAB moiety are represented by the formula:

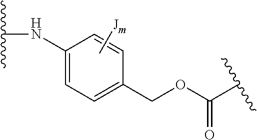

wherein subscript m is an integer ranging from 0-4, and each J is independently —C₁-C₈ alkyl, —O—(C₁-C₈ alkyl), -halogen, -nitro or -cyano.

Other examples of self-immolative groups include, but are not limited to, aromatic compounds that are electronically similar to the PAB moiety such as 2-aminoimidazol-5-methanol derivatives (Hay et al. (1999) Bioorg. Med. Chem. Lett. 9:2237) and ortho or para-aminobenzylacetals. Other RLs undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867).

In one embodiment, RL is a branched bis(hydroxymethyl)styrene (BHMS) unit.

In some embodiments, RL has the formula:

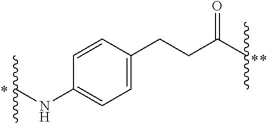 [SMILES: *Nc1ccc(CCC(*)=O)cc1]

wherein the wavy line marked with ** indicates the site of attachment to D; and the wavy line marked with * indicates the point of attachment to additional linker components of Q.

In some embodiments, RL comprises a heterocyclic “self-immolating moiety” of Formulas I, II or III bound to the drug and incorporates an amide group that upon hydrolysis by an intracellular protease initiates a reaction that ultimately cleaves the self-immolative moiety from the drug such that the drug is released from the conjugate in an active form. The linker moiety further comprises a peptide sequence adjacent to the self-immolative moiety that is a substrate for an intracellular enzyme, for example an intracellular protease such as a cathepsin (e.g., cathepsin B), that cleaves the peptide at the amide bond shared with the self-immolative moiety. For embodiments disclosed herein, a PAB-containing RL is directly attached to the tertiary hydroxyl of the lactone ring present in each of CPT1-CPT7, in each of compound 14-14z of Table I or in each of compounds 18a-18r of Table J.

In some embodiments, a heterocyclic self-immolating group (RL) is selected from Formulas I, II and III:

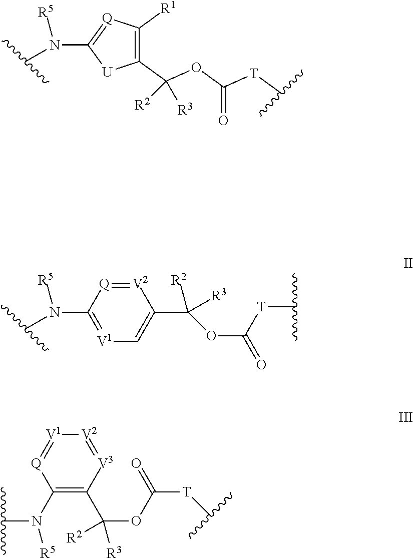

wherein the wavy lines indicate the covalent attachment sites to the cell-specific ligand and the drug moiety, and wherein U is O, S or NR⁶; Q is CR⁴ or N; V¹, V² and V³ ar independently CR⁴ or N provided that for formula II and III at least one of Q, V¹ and V² is N; T is O pending from CPT1, CPT2, CPT3, CPT4, CPT5, CPT6 or CPT7;

R¹, R², R³ and R⁴ are independently selected from the group consisting of H, F, Cl, Br, I, OH, —N(R⁵)₂, —N(R⁵)₃*, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and

R⁵ and R⁶ are independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle;

wherein C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from the group consisting of F, Cl, Br, I, OH, —N(R⁵)₂, —N(R⁵)₃+, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

The conjugate is stable extracellularly, or in the absence of an enzyme capable of cleaving the amide bond of the self-immolative moiety. However, upon entry into a cell, or exposure to a suitable enzyme, an amide bond is cleaved initiating a spontaneous self-immolative reaction resulting in the cleavage of the bond covalently linking the self-immolative moiety to the drug, to thereby effect release of the drug in its underivatized or pharmacologically active form.

The self-immolative moiety in conjugates of the invention either incorporates one or more heteroatoms and thereby provides improved solubility, improves the rate of cleavage and/or decreases propensity for aggregation of the conjugate. These improvements of the heterocyclic self-immolative linker constructs of the present invention over non-heterocyclic, PAB-type linkers in some instances result in surprising and unexpected biological properties such as increased efficacy, decreased toxicity, and/or improvements in one or more desirable pharmacokinetic and/or pharmacodynamic properties.

It is understood that T in Formulae I-III is O, as it is derived from the tertiary hydroxyl (—OH) on the lactone ring portion of any one of CPT1, CPT2, CPT3, CPT4, CPT5, CPT6, CPT7, compounds 14a-14z of Table I and compounds 18a-18r of Table J.

Not to be limited by theory or any particular mechanism, the presence of electron-withdrawing groups on the heterocyclic ring of formula I, II or III linkers sometimes moderate the rate of cleavage.

In one embodiment, the self-immolative moiety is the group of formula I in which Q is N, and U is O or S. Such a group has a non-linearity structural feature which improves solubility of the conjugates. In this context R is sometimes H, methyl, nitro, or CF₃. In one embodiment, Q is N and U is O thereby forming an oxazole ring and R is H. In another embodiment, Q is N and U is S thereby forming a thiazole ring optionally substituted at R with an Me or CF₃ group.

In another exemplary embodiment, the self-immolative moiety is the group of formula II in which Q is N and V¹ and V² are independently N or CH. In another embodiment, Q, V¹ and V² are each N. In another embodiment, Q and V¹ are N while V² is CH. In another embodiment, Q and V² are N while V¹ is CH. In another embodiment, Q and V¹ are both CH and V² is N. In another embodiment, Q is N while V¹ and V² are both CH.

In another embodiment, the self-immolative moiety is the group of formula III in which Q, V¹, V² and V³ are each independently N or CH. In another embodiment Q is N while V¹, V² and V³ are each N. In another embodiment, Q V¹, and V² are each CH while V³ is N. In another embodiment Q, V² and V³ are each CH while V¹ is N. In another embodiment, Q, V¹ and V³ are each CH while V² is N. In another embodiment, Q and V² are both N while V¹ and V³ are both CH. In another embodiment Q and V² are both CH while V¹ and V³ are both N. In another embodiment, Q and V³ are both N while V¹ and V² are both CH.

Without being bound by theory, Scheme 1a depicts a mechanism of free drug release from a Camptothecin Drug Unit attached through a nitrogen atom of an amine substituent from the free drug to a Releasable Linker that is a Glucuronide Unit.

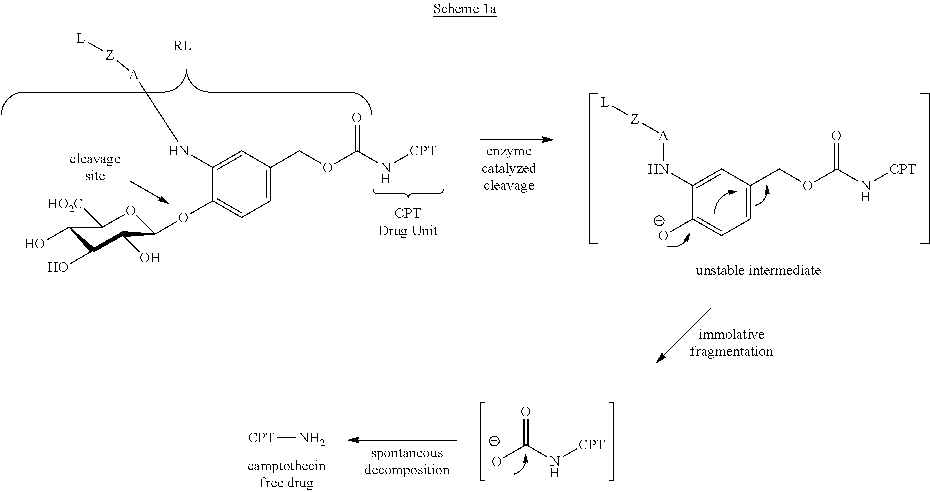

### Partitioning Agent (S*):

The ADCs described herein can also include a Partitioning Agent (S*). The Partitioning Agent portions are useful, for example, to mask the hydrophobicity of particular Camptothecin Drug Units or Linking Unit components.

Representative Partitioning Agents include polyethylene glycol (PEG) units, cyclodextrin units, polyamides, hydrophilic peptides, polysaccharides and dendrimers.

When the polyethylene glycol (PEG) units, cyclodextrin units, polyamides, hydrophilic peptides, polysaccharides or dendrimers are included in Q, the groups may be present as an ‘in line’ component or as a side chain or branched component. For those embodiments in which a branched version is present, the Linker Units will typically include a lysine residue (or Parallel Connector Unit, B) that provides simple functional conjugation of, for example, the PEG unit, to the remainder of the Linking Unit.

### Polyethylene Glycol Unit (PEG)

Polydisperse PEGS, monodisperse PEGS and discrete PEGs can be used to make the Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein comprises one or multiple polyethylene glycol chains. In some embodiments the polyethylene glycol chains are linked together, for example, in a linear, branched or star shaped configuration. Typically, at least one of the PEG chains is derivitized at one end for covalent attachment to an appropriate site on a component of the Linker Unit (e.g. B) or can be used as an in-line (e.g., bifunctional) linking group within to covalently join two of the Linker Unit components (e.g., Z-A-S*-RL-, Z-A-S*-RL-Y—). Exemplary attachments within the Linker Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment within the Linker Unit is by means of a non-conditionally cleavable linkage. In some embodiments, attachment within the Linker Unit is not via an ester linkage, hydrazone linkage, oxime linkage, or disulfide linkage. In some embodiments, attachment within the Linker Unit is not via a hydrazone linkage.

A conditionally cleavable linkage refers to a linkage that is not substantially sensitive to cleavage while circulating in the plasma but is sensitive to cleavage in an intracellular or intratumoral environment. A non-conditionally cleavable linkage is one that is not substantially sensitive to cleavage in any biological environment. Chemical hydrolysis of a hydrazone, reduction of a disulfide, and enzymatic cleavage of a peptide bond or glycosidic linkage are examples of conditionally cleavable linkages.

In some embodiments, the PEG Unit will be directly attached to a Parallel Connector Unit B. The other terminus (or termini) of the PEG Unit will be free and untethered and may take the form of a methoxy, carboxylic acid, alcohol or another suitable functional group. The methoxy, carboxylic acid, alcohol or other suitable functional group acts as a cap for the terminal PEG subunit of the PEG Unit. By untethered, it is meant that the PEG Unit will not be attached at that untethered site to a Camptothecin, to an antibody, or to another linking component. The skilled artisan will understand that the PEG Unit in addition to comprising repeating polyethylene glycol subunits may also contain non-PEG material (e.g., to facilitate coupling of multiple PEG chains to each other). Non-PEG material refers to the atoms in the PEG Unit that are not part of the repeating —CH₂CH₂O-subunits. In some embodiments provided herein, the PEG Unit comprises two monomeric PEG chains attached to each other via non-PEG elements. In other embodiments provided herein, the PEG Unit comprises two linear PEG chains attached to a central core or Parallel Connector Unit (i.e., the PEG Unit itself is branched).

There are a number of PEG attachment methods available to those skilled in the art, [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); PCT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal.alpha.-carbon of a peptide); Veronese et al., (1985) Appl. Biochem. Biotechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for attaching PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, PEG molecules may be attached to amino groups using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG2-succinimidyl succinate (mPEG₂-SS); mPEG-succinimidyl carbonate (mPEG-SC), mPEG2-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG₂-SPA); mPEG-N-hydroxy-succinimide (mPEG-NHS); mPEG2-N-hydroxy-succinimide (mPEG₂-NHS); mPEG-cyanuric chloride; mPEG2-cyanuric chloride; mPEG₂-Lysinol-NPC, and mPEG₂-Lys-NHS.

Generally, at least one of the PEG chains that make up the PEG Unit is functionalized so that it is capable of covalent attachment to other Linker Unit components.

Functionalization includes, for example, via an amine, thiol, NHS ester, maleimide, alkyne, azide, carbonyl, or some other functional group. In some embodiments, the PEG Unit further comprises non-PEG material (i.e., material not comprised of —CH₂CH₂O—) that provides coupling to other Linker Unit components or to facilitate coupling of two or more PEG chains.

The presence of the PEG Unit (or other Partitioning Agent) in the Linker Unit can have two potential impacts upon the pharmacokinetics of the resulting ADC. The desired impact is a decrease in clearance (and consequent increase in exposure) that arises from the reduction in non-specific interactions induced by the exposed hydrophobic elements of the ADC or to the Camptothecin itself. The second impact is undesired and is a decrease in volume and rate of distribution that sometimes arises from the increase in the molecular weight of the ADC.

Increasing the number of PEG subunits increases the hydrodynamic radius of a conjugate, typically resulting in decreased diffusivity. In turn, decreased diffusivity typically diminishes the ability of the ADC to penetrate a tumor (Schmidt and Wittrup, Mol Cancer Ther 2009; 8:2861-2871). Because of these two competing pharmacokinetic effects, it is desirable to use a PEG that is sufficiently large to decrease the ADC clearance thus increasing plasma exposure, but not so large as to greatly diminish its diffusivity, to an extent that it interferes with the ability of the ADC to reach the intended target cell population. See the examples (e.g., examples 1, 18, and 21) of US2016/0310612, which are incorporated by reference herein, for methodology for selecting an optimal PEG size for a particular drug-linker.

In one group of embodiments, the PEG Unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In preferred embodiments, the PEG Unit comprises a combined total of at least 4 subunits, at least 6 subunits, at least 8 subunits, at least 10 subunits, or at least 12 subunits. In some such embodiments, the PEG Unit comprises no more than a combined total of about 72 subunits, preferably no more than a combined total of about 36 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

Illustrative linear PEG Units that can be used in any of the embodiments provided herein are as follows:

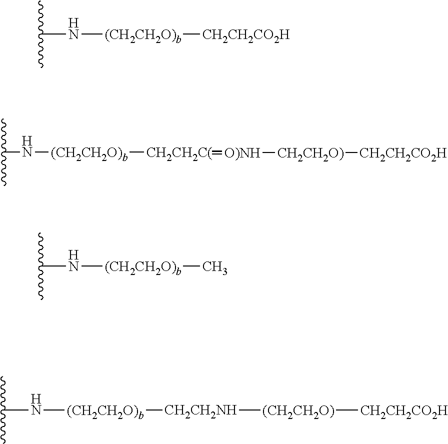

wherein the wavy line indicates site of attachment to the Parallel Connector Unit (B), and each n is independently selected from 4 to 72, 6 to 72, 8 to 72, 10 to 72, 12 to 72, 6 to 24, or 8 to 24. In some embodiments, subscript b is about 4, about 8, about 12, or about 24.

As described herein, the PEG unit is selected such that it improves clearance of the resultant ADC but does not significantly impact the ability of the Conjugate to penetrate into the tumor. In embodiments, the PEG unit to be selected for use will preferably have from 4 subunits to about 24 subunits, more preferably about 4 subunits to about 12 subunits.

In preferred embodiments of the present disclosure the PEG Unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG Unit has at least 6 subunits or at least 8, 10 or 12 subunits. In some such aspects, the PEG Unit has at least 6 subunits or at least 8, 10 or 12 subunits but no more than 72 subunits, preferably no more than 36 subunits.

It will be appreciated that when referring to PEG subunits, and depending on context, the number of subunits can represent an average number, e.g., when referring to a population of ADCs or Camptothecin-Linker Compounds using polydisperse PEGs.

### Parallel Connector Unit (B):

In some embodiments, the ADCs and Camptothecin Linker Compounds will comprise a Parallel Connector Unit to provide a point of attachment to a Partitioning Agent (shown in the Linker Units as —B(S*)—). As a general embodiment, the PEG Unit can be attached to a Parallel Connector Unit such as lysine as shown below wherein the wavy line and asterisks indicate covalent linkage within the Linker Unit of an ADC or Camptothecin Linker Compound:

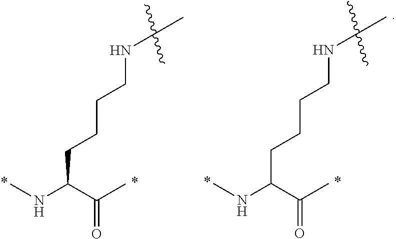 [SMILES: *NCCCC[C@H](NC)C(C)=O ; *NCCCCC(NC)C(C)=O]

### Spacer Unit (Y):

In some embodiments, the ADCs provided herein will have a Spacer (Y) between the Releasable Linker (RL) and the Camptothecin. The Spacer Unit can be a functional group to facilitate attachment of RL to the Camptothecin, or it can provide additional structural components to further facilitate release of the Camptothecin Unit from the remainder of the Conjugate (e.g., a methylene carbamate unit).

In those embodiments to further facilitate release of the Camptothecin Unit as free drug exemplary Spacer Units are represented by the formulae:

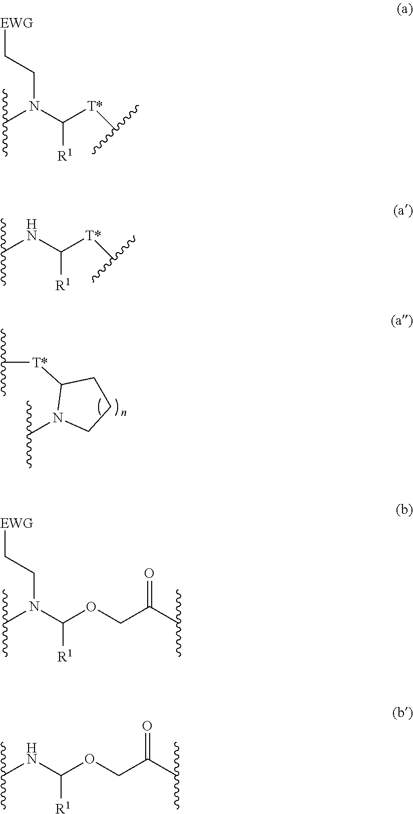

wherein EWG represents an electron-withdrawing group, R¹ is —H or C₁-C₄ alkyl and subscript n is 1 or 2. In some embodiments, EWG is selected from the group consisting of —CN, —NO₂, —CX₃, —X, ′C(═O)OR, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃, wherein X is —F, —Br, —Cl, or —I, and R′ is independently selected from the group consisting of hydrogen and C₁-C₆ alkyl, and wherein the wavy line adjacent to the nitrogen atom in each of formula (a), (a′), (a″), (b) and (b′) is the point of covalent attachment to RL and the wavy line adjacent to the carbonyl carbon atom of formula (b) and formula (b′) is the point of covalent attachment to a heteroatom of a hydroxyl or primary or secondary amine of a camptothecin compound of formula CPT1, CPT2, CPT3, CPT4, CPT5, CPT 6 or CPT7, or of any one of compounds 14a-14z of Table I or any one of compounds 18a-18r of Table J and wherein

formula (a), formula (a′) and formula (a″) represents exemplary methylene carbamate units in which T* is the heteroatom from a hydroxyl or primary or secondary amine functional group of a camptothecin compound of formula CPT1, CPT2, CPT3, CPT4, CPT5, CPT 6 or CPT7 or of any one of compounds 14a-14z of Table I or any one of compounds 18a-18r of Table J and wherein the wavy line adjacent to T* is the point of covalent attachment to the remainder of the Camptothecin Drug Unit corresponding in structure to the camptothecin compound.

In still other embodiments, Spacer Units that are methylene carbamate units are represented by the formulae:

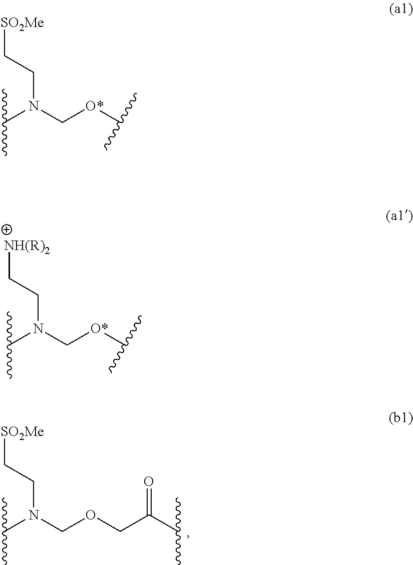

wherein formula (a1) and formula (a1′) in which each R is independently —H or C₁-C₄ alkyl represents methylene carbamate units in which O* is the oxygen atom from the hydroxyl substituent to the lactone ring of the camptothecin compound of formula CPT1, CPT2, CPT3, CPT4, CPT5, CPT 6 or CPT7 or of any one of compounds 14a-14z of Table I or any one of compounds 18a-18r of Table J, or from the another hydroxyl substituent of the camptothecin compound of formula CPT5 or CPT7 or from the hydroxyl substituents of R^{F} or R^{F′} of CPT6, when at least one of R^{F} and R^{F′} is C₁-C₈ hydroxyalkyl N,N—(C₁-C₄ hydroxyalkyl)(C₁-C₄ alkyl)-amino-C₁-C₈ alkyl- or N—C₁-C₄ hydroxyalkyl-C₁-C₈ aminoalkyl-, C₁-C₈ alkylC(O)—,

and the wavy lines of formula (a1), formula (a1′) and formula (b1) retain their previous meanings from formulae (a), (a′) and (b), respectively. In formula (a1′) the —CH₂CH₂N⁺(R)₂ moiety represents exemplary Basic Units in protonated form.

Without being bound by theory, Scheme 1b depicts a mechanism of free drug release from a Camptothecin attached to a methylene carbamate unit in an ADC having a self-immolative moiety. In that scheme, T* is a heteroatom from the hydroxyl or primary or secondary amine of a Camptothecin compound that is incorporated into the methylene carbamate unit.

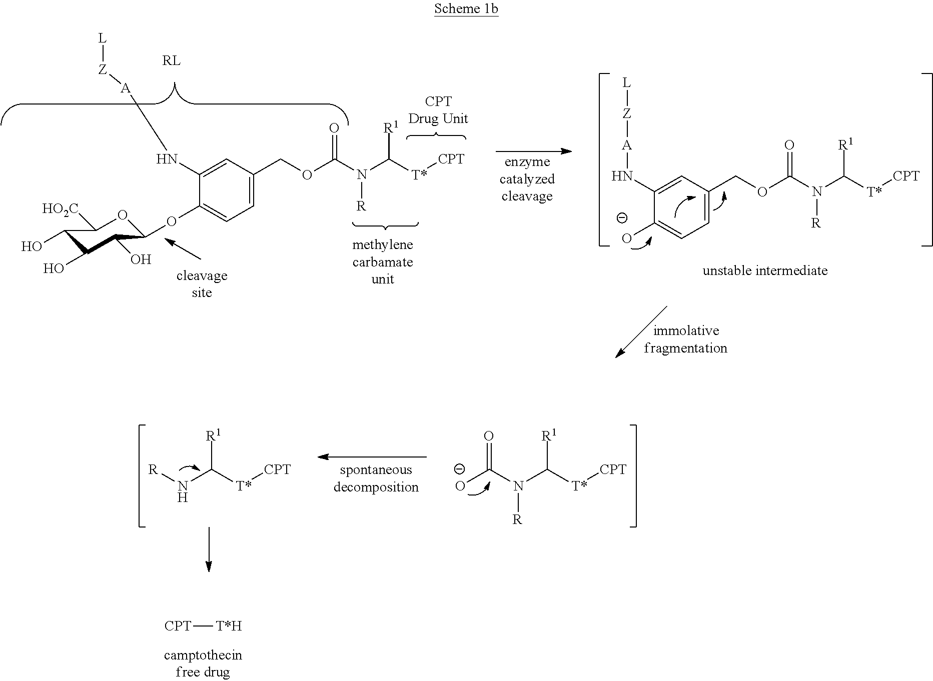

Subscript “p”—Drug to Antibody Ratio (DAR)

In one group of embodiments of the invention, subscript p represents the number of Drug Linker moieties on a Ligand Unit (e.g., antibody) of an individual ADC and is an integer preferably ranging from 1 to 16, 1 to 12, 1 to 10, or 1 to 8. Individual ADCs can also be referred to as an ADC compound. In that group of embodiments there are 1, 2, 3, 4, 5, 6, 7, 8, 9, 10, 11, 12, 13, 14, 15, or 16 Drug Linker moieties conjugated to a Ligand Unit (e.g., antibody) of an individual ADC. In another group of embodiments of the invention, an ADC describes a population of individual ADC compounds substantially identical except for the number of Camptothecin drug linker moieties bound to each Ligand Unit (i.e., an ADC composition) so that subscript p represents the average number of Camptothecin drug linker moieties bound to the Ligand Units of the ADC composition. In that group of embodiments, subscript p, which represents DAR, is a number ranging from 1 to about 16, 1 to about 12, 1 to about 10, or 1 to about 8, from 2 to about 16, 2 to about 12, 2 to about 10, or 2 to about 8. In some embodiments, the value of subscript p refers to the average drug loading as well as the drug loading of the predominate ADC in the composition. In some embodiments, the value of subscript p refers to the predominate drug loading of the ADC in the composition. In some embodiments, at least about 60%, such as at least about any of 70%, 80%, 90%, 95%, 99%, 99.9%, or 100% of the ADC in the composition has the value of subscript p (i.e., DAR) as the drug loading. For example, in some embodiments, ADC with a DAR of 8 may refer to a composition wherein the predominate ADC has a DAR of 8 (e.g., at least about any of 60% 70%, 80%, 90%, 95%, 99%, 99.9%, or 100% of the ADC has a DAR of 8) and wherein there may be small amount (e.g., no more than about any of 40%, 30%, 20%, 10%, 5%, 2%, 1%, or 0.1%) of ADC with other DARs (e.g., aDAR of 8, 7, 6, 5, or 4).

In some embodiments, conjugation will be via the interchain disulfides and there will from 1 to about 8 Camptothecin Linker Compound molecules conjugated to a targeting agent that becomes a Ligand Unit. In some embodiments, conjugation will be via an introduced cysteine residue as well as interchain disulfides and there will be from 1 to 10 or 1 to 12 or 1 to 14 or 1 to 16 Camptothecin Linker Compound moieties conjugated to a Ligand Unit (e.g., antibody). In some embodiments, conjugation will be via an introduced cysteine residue and there will be 4 or 8 Camptothecin Linker Compound molecules conjugated to a Ligand Unit (e.g., antibody).

TABLE 4
Camptothecin Drug-Linker Compounds

| | | | | | |
|---|---|---|---|---|---|
| | | | | | Campto- |
| Drug | | | | | thecin |
| Linker | | | | | (N or |
| Number | Z′-A | S* or B(S*) | RL | Y | O link) |

Glucuronide Drug-Linker Compounds

| | | | | | |
|---|---|---|---|---|---|
| 67 | mDPr-β-Ala | — | Glucuronide | — | CPT1 (N) |
| 72 | mDPr- | Lys (PEG24)- | Glucuronide | — | CPT1 (N) |
| | | β-Ala | | | |
| 51 | mPr-β-Ala | — | Glucuronide | — | ″ |
| 29 | mPr-β-Ala | — | Glucuronide | — | CPT4 (N) |
| 48 | mPr-β-Ala | — | Glucuronide | — | 13b (N) |
| 100 | mDPr-MeGly- | — | Glucuronide | — | CPT4 (N) |
| 55 | mPr-MeGly- | — | Glucuronide | (a1) | CPT2 (O) |
| 34 | mPr-MeGly- | — | Glucuronide | (a1) | CPT5 (O) |
| 42 | mPr-MeGly- | — | Glucuronide | (b1) | CPT4 (N) |
| 103 | mPr-β-Ala | — | Glucuronide | — | CPT6 (N) |
| 109a | mPr-MeGly- | — | Glucuronide | — | CPT1 (N) |
| 109b | mPr-MeGly- | — | Glucuronide | — | 18q (N) |
| 58 | mPr-MeGly- | — | Glucuronide | (a1) | CPT3 (O) |
| 61 | mPr-MeGly- | — | Glucuronide | (a1) | CPT3 (O′) |
| 150 | mPr-MeGly | — | Glucuronide | (a1) | CPT7 (O′) |
| 135 | mPr-β-Ala | — | Glucuronide | — | 14a (N) |
| 138a | mPr-β-Ala | — | Glucuronide | — | 18q (N) |
| 138b | mPr-β-Ala | — | Glucuronide† | — | 18r (N) |
| 141 | mPr-MeGly | — | Glucuronide | (a1) | 18m (O) |
| 113 | mPr-MeGly | — | Glucuronide | — | 18m (O) |
| 114a | mPr-MeGly | — | Glucuronide | — | 18h (O) |
| 114b | mPr-MeGly | — | Glucuronide | — | 6 (O) |
| 150 | mPr-MeGly | — | Glucuronide | (a) | CPT7 (O′) |

Click Drug Linker Compounds

| | | | | | |
|---|---|---|---|---|---|
| 86 | PropargOPr- | — | Glucuronide | (a1) | CPT4 (O′) |
| | MeGly- | | | | |
| 82 | PropargOPr- | — | Glucuronide | — | CPT4 (N) |
| | MeGly- | | | | |
| 87 | PropargOPr- | — | Glucuronide | (a1) | CPT4 (O′) |
| | MeGly- | | | | |
| 63 | PropargOPr- | — | Glucuronide | (a1) | CPT3 (O′) |
| | MeGly- | | | | |
| 62 | PropargOPr- | — | Glucuronide | (a1) | CPT3 (O) |
| | MeGly- | | | | |
| 144 | PropargOPr- | — | Glucuronide | (a1) | CPT6 (N) |
| | MeGly- | | | | |

†Glucuronic acid residue replaced by mannose 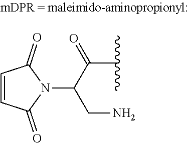 [SMILES: *C(=O)C(CN)N1C(=O)C=CC1=O] 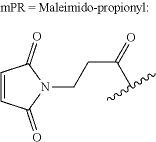 [SMILES: *C(=O)CCN1C(=O)C=CC1=O]
PropargOpr = —(C═O)CH₂CH₂OCH₂C≡CH

TABLE 5
Other Camptothecin Drug Linker Compounds

| Drug | | |
|---|---|---|
| Linker | General | |
| Number | Formula | Compound Structure |
| 120 | Z′-A-D | 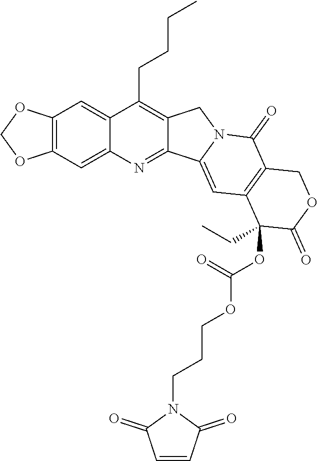 [SMILES: CCCCc1c2c(nc3cc4c(cc13)OCO4)-c1cc3c(c(=O)n1C2)COC(=O)[C@@]3(CC)OC(=O)OCCCN1C(=O)C=CC1=O] |
| 124 | Z′-A-D | 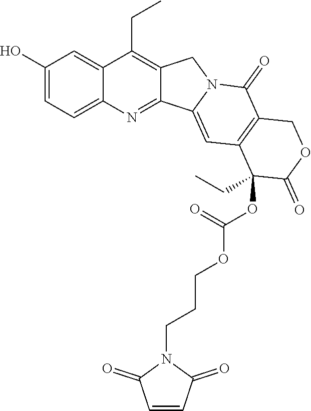 [SMILES: CCc1c2c(nc3ccc(O)cc13)-c1cc3c(c(=O)n1C2)COC(=O)[C@@]3(CC)OC(=O)OCCCN1C(=O)C=CC1=O] |
| 120 | Z′-A-S*-W-D | 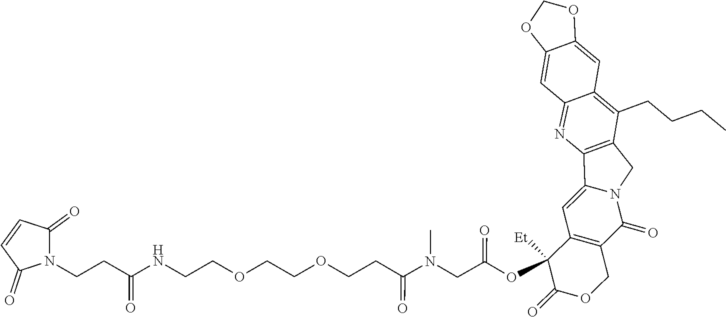 [SMILES: CCCCc1c2c(nc3cc4c(cc13)OCO4)-c1cc3c(c(=O)n1C2)COC(=O)[C@@]3(CC)OC(=O)CN(C)C(=O)CCOCCOCCNC(=O)CCN1C(=O)C=CC1=O] |
| 133 | Z′-A-S*-W-RL-D | 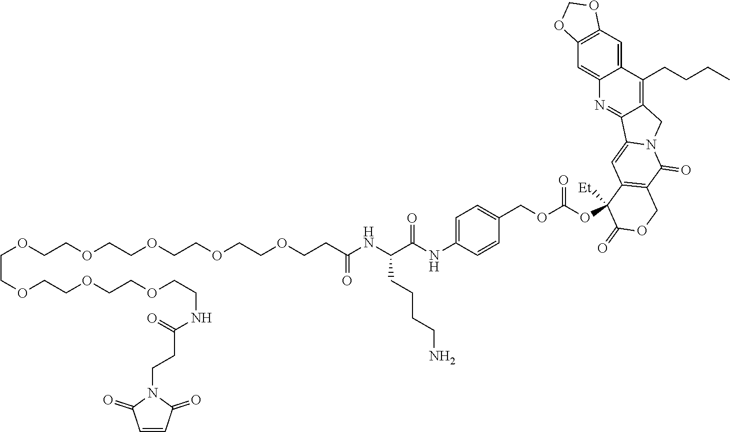 [SMILES: CCCCc1c2c(nc3cc4c(cc13)OCO4)-c1cc3c(c(=O)n1C2)COC(=O)[C@@]3(CC)OC(=O)OCc1ccc(NC(=O)[C@H](CCCCN)NC(=O)CCOCCOCCOCCOCCOCCOCCOCCOCCNC(=O)CCN2C(=O)C=CC2=O)cc1] |
| 129 | Z′-A-S*-W-RL-D | 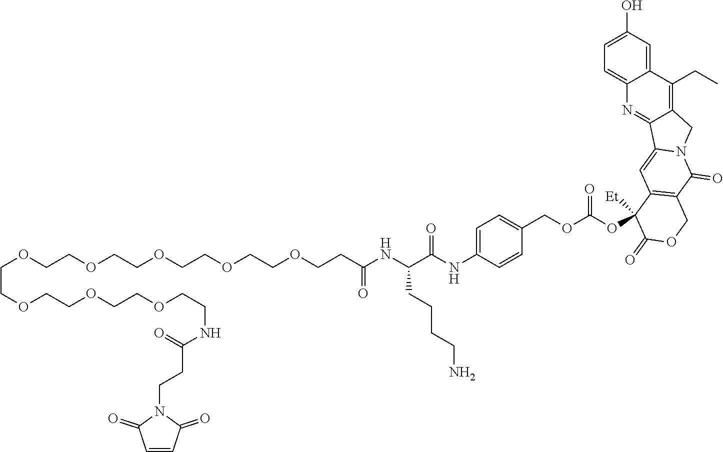 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCCN)NC(=O)CCOCCOCCOCCOCCOCCOCCOCCOCCNC(=O)CCN2C(=O)C=CC2=O)cc1] |

In some embodiments, provided herein is an antibody-drug conjugate having the formula of

L-(Q-D)ₚ

or a salt thereof, wherein
L is a Ligand Unit comprising an antibody or antigen binding fragment thereof that bonds to CEACAM5 comprising
a CDR1-H comprising the amino acid sequence set forth in SEQ ID NO:1;
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:6;
subscript p is an integer ranging from 1 to 16;

### Q is a Linker Unit;

and D is a Drug Unit having the formula of:

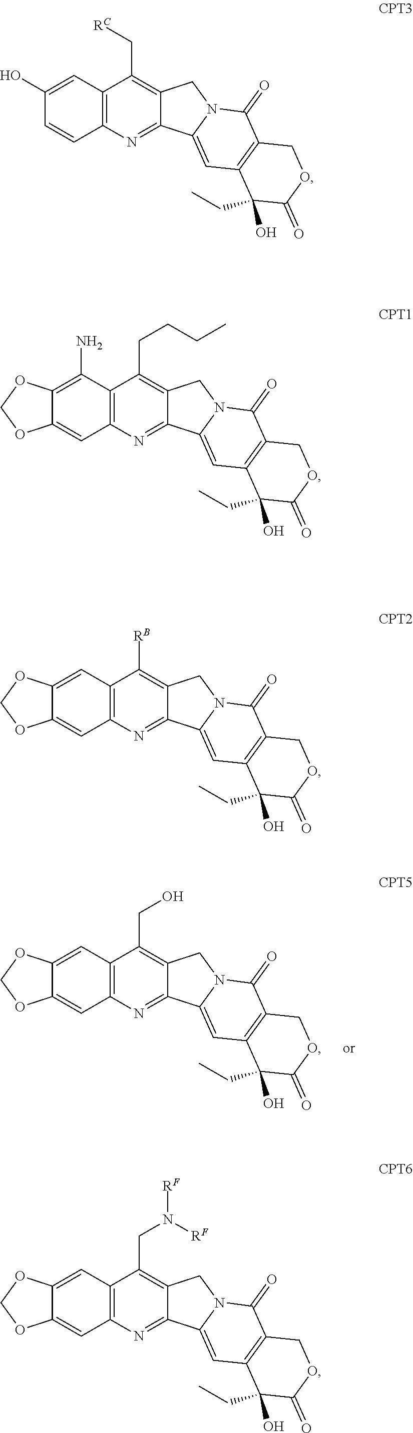

wherein R^{B} is a member selected from the group consisting of H, C₁-C₈ alkyl, C₁-C₈ haloalkyl, C₃-C₈ cycloalkyl, (C₃-C₈ cycloalkyl)-C₁-C₄ alkyl-, phenyl and phenyl-C₁-C₄ alkyl-;

R^{C} is a member selected from the group consisting of C₁-C₆ alkyl and C₃-C₆ cycloalkyl;

each R^{F} and R^{F′} is a member independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-, or

R^{F} and R^{F′} are combined with the nitrogen atom to which each is attached to form a 5-, 6- or 7-membered ring having 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and wherein the cycloalkyl, heterocycloalkyl, phenyl and heteroaryl portions of R^{B}, R^{C}, R^{F} and R^{F′} are substituted with from 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and

wherein the point of attachment of D to Q is through the heteroatom of any one of the hydroxyl or primary or secondary amine functional groups present on CPT1, CPT2, CPT3, CPT5, CPT6 or CPT7 when Q is —Z-A-RL-, —Z-A-RL-Y—, —Z-A-S*-RL-, —Z-A-B(S*)—RL-, —Z-A-S*-RL-Y— or —Z-A-B(S*)—RL-Y— in which RL is any one of the Releasable Linkers disclosed herein, or

wherein the point of attachment of D to Q is through the oxygen atom of the hydroxyl group substituent in the lactone ring of CPT1, CPT2, CPT3, CPT5, CPT6 or CPT7 when Q is —Z-A-, —Z-A-S*—W— or —Z-A- B(S*)—W—, or when Q is —Z-A-S*-RL-, —Z-A-B(S*)—RL-, —Z-A —S*—W-RL-, or —Z-A-B(S*)—W-RL- in which RL is a Releasable Unit other than a Glucuronide Unit; and

provided that at least one of R^{F} and R^{F′} is —H, when the point of attachment is to the nitrogen atom of the amino group of CPT6, and
provided that —Z-A- of —Z-A-RL-, —Z-A-RL-Y—, —Z A-S*-RL-, —Z-A-B(S*)—RL-, —Z-A-S*-RL-Y— and —Z-A-B(S*)—RL-Y— is other than succinimido-caproyl-β-alanyl, optionally having the succinimide ring in hydrolyzed form, when D is CPT1 having attachment through its amino group, wherein the wavy line indicates the site of covalent attachment to Q.

In some embodiments, provided herein is an antibody-drug conjugate having the formula of

L-(Q-D)ₚ

or a salt thereof, wherein
L is a Ligand Unit comprising an antibody or antigen binding fragment thereof that binds to CEACAM5 comprising
a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8;
subscript p is an integer ranging from 1 to 16;

### Q is a Linker Unit;

and D is a Drug Unit having the formula of:

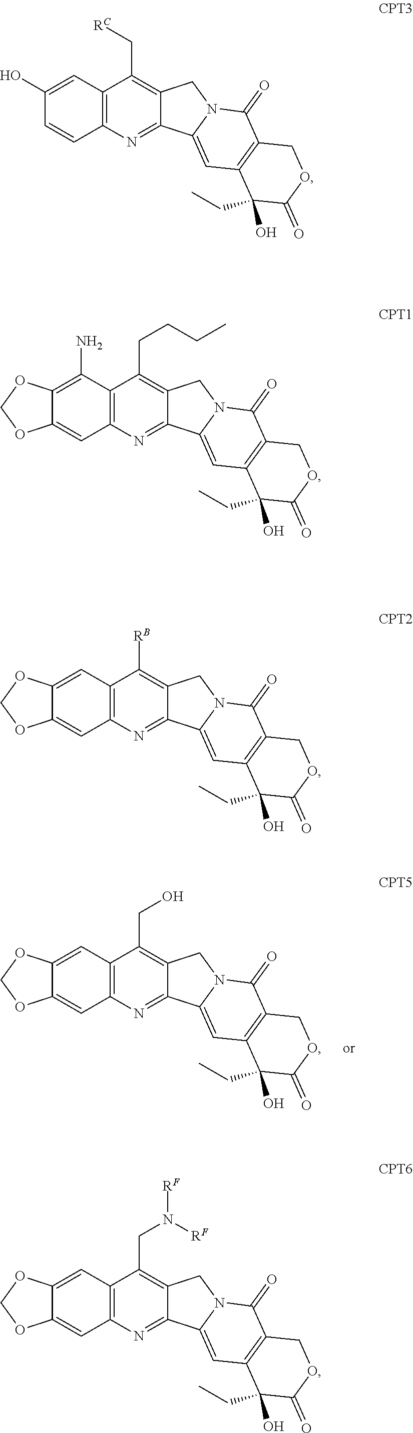

wherein R^{B} is a member selected from the group consisting of H, C₁-C₈ alkyl, C₁-C₈ haloalkyl, C₃-C₈ cycloalkyl, (C₃-C₈ cycloalkyl)-C₁-C₄ alkyl-, phenyl and phenyl-C₁-C₄ alkyl-;

R^{C} is a member selected from the group consisting of C₁-C₆ alkyl and C₃-C₆ cycloalkyl;

each R^{F} and R^{F′} is a member independently selected from the group consisting of —H, C₁-C₈ alkyl, C₁-C₈ hydroxyalkyl, C₁-C₈ aminoalkyl, (C₁-C₄ alkylamino)-C₁-C₈ alkyl-, N,N—(C₁-C₄ hydroxyalkyl)(C₁-C₄ alkyl)amino-C₁-C₈ alkyl-, N,N-di(C₁-C₄ alkyl)amino-C₁-C₈ alkyl-, N—(C₁-C₄ hydroxyalkyl)-C₁-C₈ aminoalkyl, C₁-C₈ alkyl-C(O)—, C₁-C₈ hydoxyalkyl-C(O)—, C₁-C₈ aminoalkyl-C(O)—, C₃-C₁₀ cycloalkyl, (C₃-C₁₀ cycloalkyl)-C₁-C₄ alkyl-, C₃-C₁₀ heterocycloalkyl, (C₃-C₁₀ heterocycloalkyl)-C₁-C₄ alkyl-, phenyl, phenyl-C₁-C₄ alkyl-, diphenyl-C₁-C₄ alkyl-, heteroaryl and heteroaryl-C₁-C₄ alkyl-, or

R^{F} and R^{F′} are combined with the nitrogen atom to which each is attached to form a 5-, 6- or 7-membered ring having 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and wherein the cycloalkyl, heterocycloalkyl, phenyl and heteroaryl portions of R^{B}, R^{C}, R^{F} and R^{F′} are substituted with from 0 to 3 substituents selected from the group consisting of halogen, C₁-C₄ alkyl, —OH, —OC₁-C₄ alkyl, —NH₂, —NHC₁-C₄ alkyl and —N(C₁-C₄ alkyl)₂; and

wherein the point of attachment of D to Q is through the heteroatom of any one of the hydroxyl or primary or secondary amine functional groups present on CPT1, CPT2, CPT3, CPT5, CPT6 or CPT7 when Q is —Z-A-RL-, —Z-A-RL-Y—, —Z-A-S*-RL-, —Z-A-B(S*)—RL-, —Z-A-S*-RL-Y— or —Z-A-B(S*)—RL-Y— in which RL is any one of the Releasable Linkers disclosed herein, or

wherein the point of attachment of D to Q is through the oxygen atom of the hydroxyl group substituent in the lactone ring of CPT1, CPT2, CPT3, CPT5, CPT6 or CPT7 when Q is —Z-A-, —Z-A-S*—W— or —Z-A- B(S*)—W—, or when Q is —Z-A-S*-RL-, —Z-A-B(S*)—RL-, —Z-A —S*—W-RL-, or —Z-A-B(S*)—W-RL- in which RL is a Releasable Unit other than a Glucuronide Unit; and

provided that at least one of R^{F} and R^{F′} is —H, when the point of attachment is to the nitrogen atom of the amino group of CPT6, and

provided that —Z-A- of —Z-A-RL-, —Z-A-RL-Y—, —Z A-S*-RL-, —Z-A-B(S*)—RL-, —Z-A-S*-RL-Y— and —Z-A-B(S*)—RL-Y— is other than succinimido-caproyl-β-alanyl, optionally having the succinimide ring in hydrolyzed form, when D is CPT1 having attachment through its amino group, wherein the wavy line indicates the site of covalent attachment to Q.

In some embodiments, provided herein is an antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5;
subscript p is an integer ranging from 1 to 16;
Q is a Linker Unit having a formula selected from the group consisting of:

—Z-A-RL-,—Z-A-RL-Y—,—Z-A-S*-RL-,—Z-A-B(S*)—RL-, —Z-A-S*-RL-Y—, and —Z-A-B(S*)—RL-Y—;

wherein Z is a Stretcher Unit;
A is a bond or a Connector Unit;

### B is a Parallel Connector Unit;

### S* is a Partitioning Agent;

RL is a glycoside unit;

### Y is a Spacer Unit; and

D is a Drug Unit having the formula of:

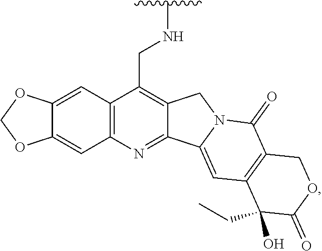 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q; and wherein the antibody or antigen binding fragment thereof comprises

a CDR1-H comprising the amino acid sequence set forth in SEQ ID NO:1

a CDR2-H comprising the amino acid sequence set forth in SEQ ID NO:2;

a CDR3-H comprising the amino acid sequence set forth in SEQ ID NO:3;

a CDR1-L comprising the amino acid sequence set forth in SEQ ID NO:4;

a CDR2-L comprising the amino acid sequence NTR; and

a CDR3-L comprising the amino acid sequence set forth in SEQ ID NO:6.

In some embodiments, the ADC described herein has the formula:

L-(Q-D)₈,

wherein L comprises an antibody or antigen binding fragment thereof that binds to CEACAM5 comprising:

a CDR1-H comprising the amino acid sequence set forth in SEQ ID NO:1

a CDR2-H comprising the amino acid sequence set forth in SEQ ID NO:2;

a CDR3-H comprising the amino acid sequence set forth in SEQ ID NO:3;

a CDR1-L comprising the amino acid sequence set forth in SEQ ID NO:4;

a CDR2-L comprising the amino acid sequence NTR; and

a CDR3-L comprising the amino acid sequence set forth in SEQ ID NO:6;

and wherein Q-D is

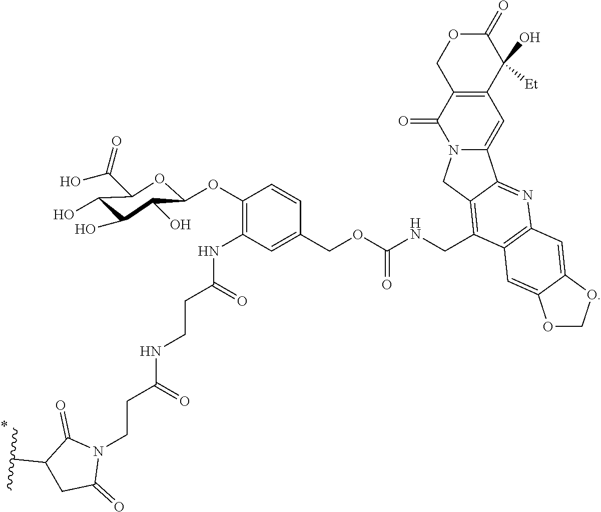

In some embodiments, provided herein is an antibody-drug conjugate or salt thereof, having the formula:

L-(Q-D)₈,

wherein L comprises an antibody or antigen binding fragment thereof that binds to CEACAM5 comprising:

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8;

and wherein Q-D is

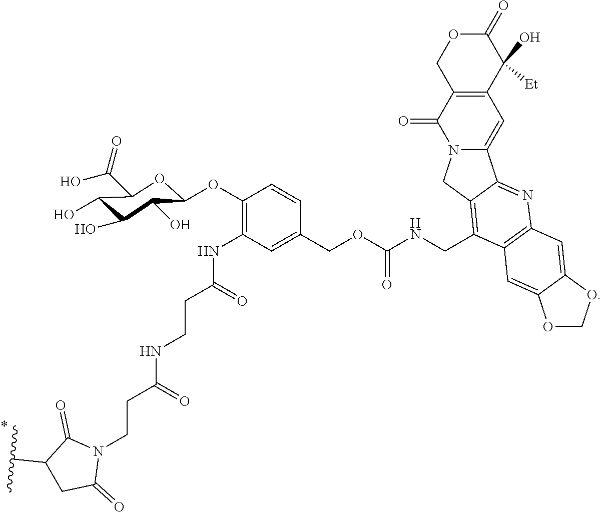

In some embodiments, provided herein is an antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5 comprising:
a CDR1-H comprising the amino acid sequence set forth in SEQ ID NO:1
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:6;
subscript p is an integer ranging from 1 to 16;

### Q is a Linker Unit;

and D is a Drug Unit, wherein the Drug Unit is a Topoisomerase I inhibitor.

In some embodiments, provided herein is an antibody-drug conjugate or salt thereof, having the formula:

L-(Q-D)₈,

wherein L comprises an antibody or antigen binding fragment thereof that binds to CEACAM5 comprising:

a heavy chain that comprises the amino acid sequence set forth in SEQ ID NO:9 and

a light chain that comprises the amino acid sequence set forth in SEQ ID NO:10;

Q is a Linker Unit;

and D is a Drug Unit, wherein the Drug Unit is a Topoisomerase I inhibitor.

In some embodiments, provided herein is an antibody-drug conjugate or salt thereof, having the formula:

L-(Q-D)₈,

wherein L comprises an antibody or antigen binding fragment thereof that binds to CEACAM5 comprising:

a heavy chain that comprises the amino acid sequence set forth in SEQ ID NO:9 and

a light chain that comprises the amino acid sequence set forth in SEQ ID NO:10; and wherein Q-D is

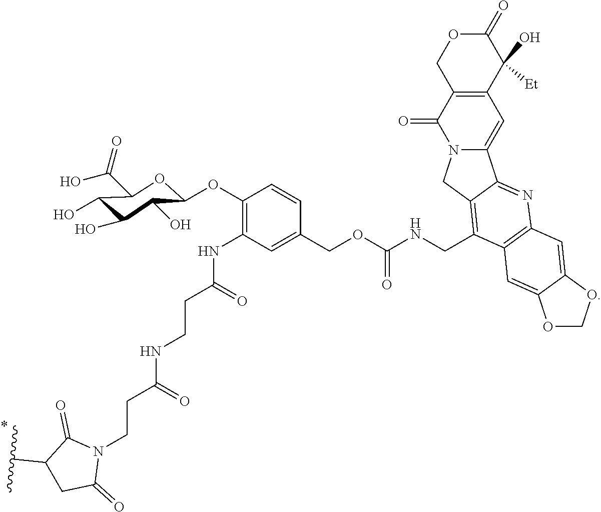

In some embodiments, provided herein is an antibody-drug conjugate or salt thereof, having the formula:

L-(Q-D)₈,

wherein L comprises an antibody or antigen binding fragment thereof that binds to CEACAM5 comprising:

a heavy chain that has the amino acid sequence set forth in SEQ ID NO:9 and

a light chain that has the amino acid sequence set forth in SEQ ID NO: 10;

and wherein Q-D is

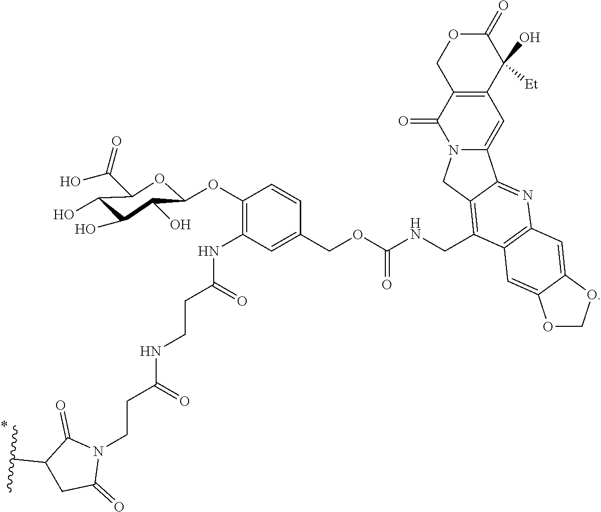

### C. Methods of Preparing ADCs

The ADCs described herein are prepared in either a serial construction of antibodies, linkers, and drug units, or in a convergent fashion by assembling portions followed by a completed assembly step. The Curtius Rearrangement or a Chloramine synthesis can be used to provide a methylene carbamate linker (Spacer) which is useful in a number of embodiments of the Conjugates described herein.

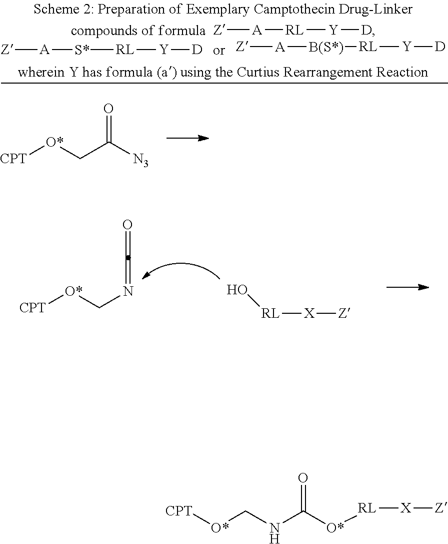

Scheme 2 illustrates a synthetic strategy involving a Curtius rearrangement of an acyl azide derivative of the free drug, wherein CPT is a Camptothecin Drug Unit corresponding in structure to a Camptothecin compound having a hydroxyl functional group whose oxygen atom, which is represented by O*, is incorporated into the methylene carbamate unit formed as a consequence of the rearrangement, Z′ is a Stretcher Unit precursor, RL is a Releasable Linker and X is -A-, -A-S*— or -A-B(S*)— wherein A is a Connector Unit, S* is a Partitioning agent and B is a Parallel Connector Unit. That strategy may be applied to Camptothecin drugs containing multiple alcohols, or other heteroatoms, as a means for acquiring regioselectivity, as there a many complementary methods of alkylation to form an acyl azide such as: halo ester alkylation, halo acid alkylation or metal carbene insertion with ethyl or methyl diazoacetate, see Doyle, M. et al. Modern Catalytic Methods for Organic Synthesis with Diazo Compounds; Wiley: New York, 1998. The acyl azide is then heated with at least a stoichiometric amount of alcohol-containing Linker Unit intermediate of formula Z′—X-RL-OH. PGP-117.0

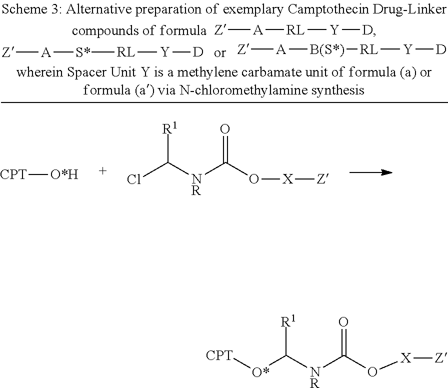

wherein R¹ is hydrogen or C₁-C₄ alkyl, R is —H or —CH₂CH₂SO₂Me and the other the variable groups have their meanings from Scheme 2.

The N-chloromethylamine synthesis is an alternative to the Curtius rearrangement in that it allows for the introduction of an unmodified alcohol or other heteroatom containing Camptothecin compound, whose use may not be compatible with the conditions required to form the acyl azide of Scheme 2, and proceeds by condensation with a reactive N-chloromethylamine. That methodology is also more appropriate for introducing certain types of methylene carbamate units as shown for example by Scheme 4.

Scheme 4 demonstrates synthesis of exemplary Camptothecin-Linker Compounds of formula Z′-A-RL-Y-D, Z′-A-S*-RL-Y-D or Z′-A-B(S*)—RL-Y-D wherein the Spacer Unit (Y) is a methylene carbamate unit of formula (a″). Reaction of the p-nitro-phenyl carbonate with the cyclic aminol provides a carbamate, which is then converted to the chlorcycloalkylamine for alkylation with a nucleophile from the thiol, hydroxyl, amine or amide functional group of free camptothecin drug. Alternatively, the carbamate can be treated with acid in the presence of the drug moiety to assemble the drug-linker intermediate shown. The alkylation product is deprotected followed by condensation of the resulting free amine with 3-maleimidopropionic acid N-hydroxysuccimide ester, which introduces a Stretcher Unit precursor covalently attached to a Connector Unit thus providing Camptothecin-Linker Compounds. The resulting Camptothecin-Linker Compounds are then condensed with a thiol-containing targeting agent to provide ADCs having a Spacer Unit comprising a self-immolative moiety and the methylene carbamate unit of formula a″.

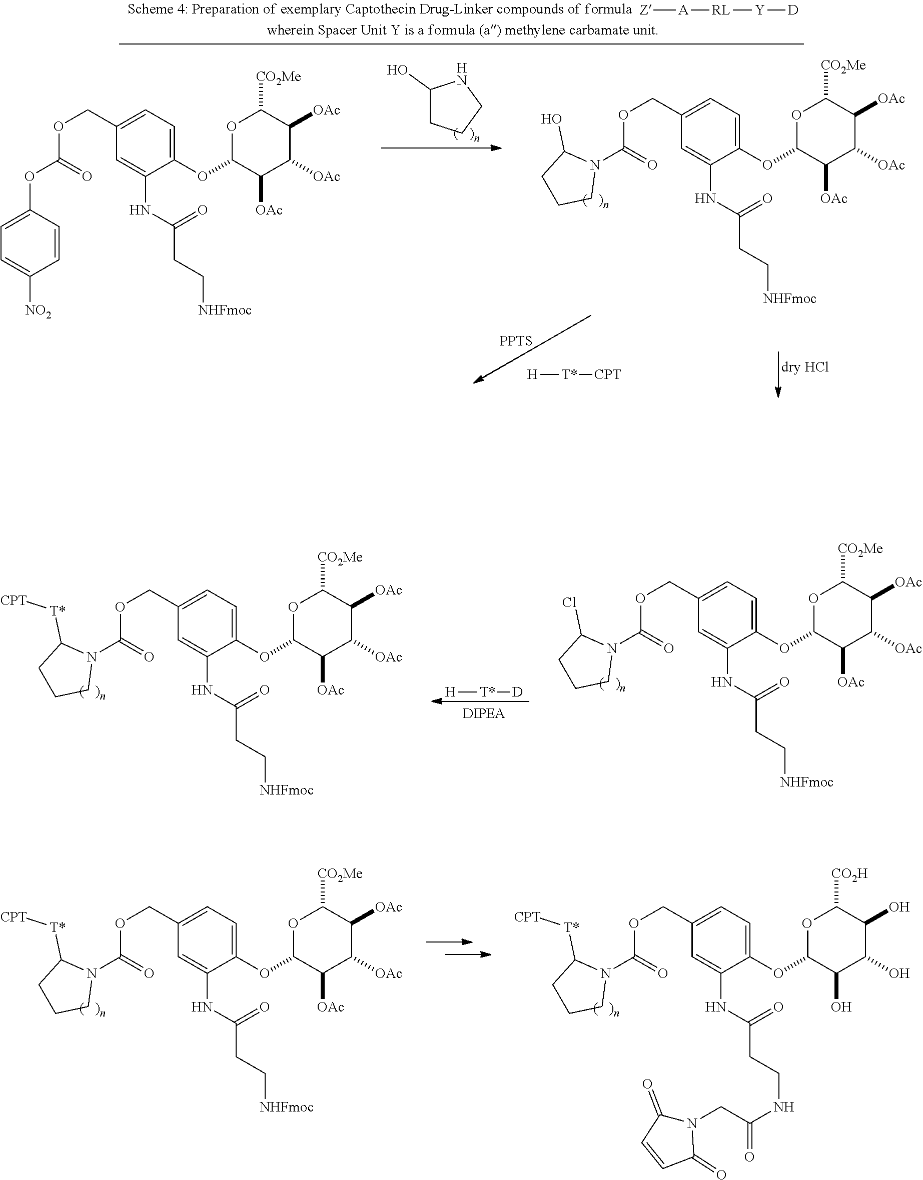

For Camptothecin-Linker Compounds and ADCs having a methylene carbamate unit wherein T* is the nitrogen atom from a primary or secondary amine substituent of a Camptothecin compound direct alkylation with a chlormethylamine following the generalized procedures provided by Scheme 3 or Scheme 4 may not be suitable due to excessive or undesired over-alkylation of the nitrogen heteroatom from the amine functional group of free drug. In those instances, the method embodied by Scheme 5 may be used.

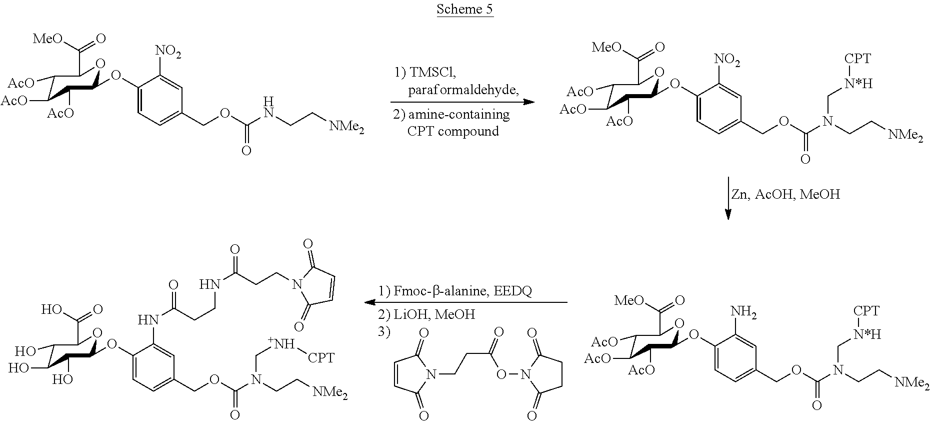

In Scheme 5 an intermediate carbamate is prepared already having a Basic Unit (i.e., the dimethylaminoethyl moiety) as the R substituent for a formula (a1′) methylene carbamate unit. The nitrogen of that carbamate is condensed with formaldehyde and the resulting intermediate quenched with the amine functional group of an aliphatic amine-containing camptothecin drug. N* represents the nitrogen atom from that functional group. That condensation forms the methylene carbamate of formula (a1′) covalently attached to a Drug Unit, wherein R¹ is hydrogen and R is dimethylaminoethyl. The phenyl nitro group is then reduced to an amine in order to provide a handle for sequential introduction of a Connector Unit (A) and a Stretcher Unit precursor (Z′).

### IV. Antigen Binding Protein Expression and Production

A. Nucleic Acid Molecules Encoding Antigen Binding Proteins

Nucleic acid molecules that encode for the antigen binding proteins described herein, or portions thereof, are also provided. Such nucleic acids include, for example: 1) those encoding an antigen binding protein (e.g., an antibody or a fragment thereof), or a derivative, or variant thereof, 2) polynucleotides encoding a heavy and/or light chain, VH and/or VL domains, or 1 or more of the HVRs or CDRs located within a variable domain (e.g., 1, 2 or all 3 of the VH HVRs or CDRs or 1, 2 or all 3 of the VL HVRs or CDRs); 3) polynucleotides sufficient for use as hybridization probes, PCR primers or sequencing primers for identifying, analyzing, mutating or amplifying such encoding polynucleotides; 4) anti-sense nucleic acids for inhibiting expression of such encoding polynucleotides, and 5) complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, or 1,000 or more nucleotides in length, and/or can comprise one or more additional sequences, for example, regulatory sequences, and/or be part of a larger nucleic acid, for example, a vector. The nucleic acids can be single-stranded or double-stranded.

The nucleic acid molecules can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and may or may not contain intronic sequences. In certain embodiments, the nucleic acid is a cDNA molecule.

Thus, nucleic acid molecules comprising polynucleotides that encode one or more chains of an anti-CEACAM5 antibodies, are provided. In some embodiments, a nucleic acid molecule comprises a polynucleotide that encodes a heavy chain or a light chain of an anti-CEACAM antibody. In some embodiments, a nucleic acid molecule comprises both a polynucleotide sequence that encodes a heavy chain and a polynucleotide sequence that encodes a light chain, of an anti-CEACAM5 antibody). In some embodiments, a first nucleic acid molecule comprises a first polynucleotide sequence that encodes a heavy chain and a second nucleic acid molecule comprises a second polynucleotide sequence that encodes a light chain.

In one embodiment, the nucleic acid molecule comprises a polynucleotide encoding the VH of one of the antibodies provided herein. In another embodiment, the nucleic acid comprises a polynucleotide encoding the VL of one of the antibodies provided herein. In still another embodiment, the nucleic acid encodes both the VH and the VL of one of the antibodies provided herein. In some embodiments, the nucleic acid encodes an antibody VH comprising the amino acid sequence set forth in SEQ ID NO.7 and a VL comprising the amino acid sequence set forth in SEQ ID NO.8.

In a particular embodiment, the nucleic acid encodes a variant of one or more of the above amino acid sequences (e.g., the heavy chain and/or light chain amino acid sequences, or the VH and/or VL amino acid sequences disclosed herein), wherein the variants has at most 25 amino acid modifications, such as at most 20, such as at most 15, 14, 13, 12 or 11 amino acid modifications, such as 10, 9, 8, 7, 6, 5, 4, 3, 2 or 1 amino-acid modifications, such as deletions or insertions, preferably substitutions, such as conservative substitutions.

Once nucleic acids encoding VH and VL segments are obtained, these nucleic acids can be further manipulated by standard recombinant DNA techniques, for example to convert the variable region genes to full-length antibody chain genes, to Fab fragment genes or to a scFv gene. In these manipulations, a VL- or VH-encoding nucleic acid is operatively linked to another nucleic acid encoding another polypeptide, such as an antibody constant region or a flexible linker.

The isolated nucleic acid encoding the VH region can be converted to a full-length heavy chain gene by operatively linking the VH-encoding nucleic acid to another nucleic acid molecule encoding heavy chain constant regions (hinge, CH1, CH₂ and/or CH₃). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding nucleic can be operatively linked to another nucleic acid molecule encoding only the heavy chain CH1 constant region.

The isolated nucleic acid molecule encoding the VL region can be converted to a full-length light chain gene (as well as a Fab light chain gene) by operatively linking the VL-encoding nucleic acid molecule to another nucleic acid molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The light chain constant region can be a kappa or lambda constant region.

To create a scFv gene, the VH- and VL-encoding nucleic acid fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

In another aspect, nucleic acid molecules that are suitable for use as primers or hybridization probes for the detection of nucleic acid sequences are also provided. A nucleic acid molecule can comprise only a portion of a nucleic acid sequence encoding a full-length polypeptide, for example, a fragment that can be used as a probe or primer or a fragment encoding an active portion (e.g., CEACAM5 binding portion) of a polypeptide.

Probes based on the sequence of a nucleic acid can be used to detect the nucleic acid or similar nucleic acids, for example, transcripts encoding a polypeptide. The probe can comprise a label group, e.g., a radioisotope, a fluorescent compound, an enzyme, or an enzyme co-factor. Such probes can be used to identify a cell that expresses the polypeptide.

Vectors, including expression vectors, comprising one or more nucleic acids encoding one or more components of the antibody or antigen binding fragment thereof (e.g. VH and/or VL; and light chains, and/or heavy chains) are also provided. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

The expression vector can also include a secretory signal peptide sequence that is operably linked to the coding sequence of interest, such that the expressed polypeptide can be secreted by the recombinant host cell, for more facile isolation of the polypeptide of interest from the cell, if desired. Other signal or secretory peptides are known to those of skill in the art and may be fused to any of the variable region polypeptide chains, for example, to facilitate or optimize expression in particular host cells.

Expression and cloning vectors of the invention will typically contain a promoter that is recognized by the host organism and operably linked to the molecule encoding the polypeptide. A large number of promoters, recognized by a variety of potential host cells, are well known. A suitable promoter is operably linked to the DNA encoding e.g., heavy chain, light chain, or other component of the antibodies and antigen-binding fragments of the invention, by removing the promoter from the source DNA by restriction enzyme digestion and inserting the desired promoter sequence into the vector. Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus serotypes 2, 8, or 9), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional specific promoters that can be utilized include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene (Prinster et a1., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25).

In certain embodiments, nucleic acids encoding the different components of the antibody or antigen binding fragment thereof can be inserted into the same expression vector. For instance, the nucleic acid encoding an anti-CEACAM5 antibody light chain or variable region can be cloned into the same vector as the nucleic acid encoding an anti-CEACAM5 antibody heavy chain or variable region. In such embodiments, the two nucleic acids may be separated by an internal ribosome entry site (IRES) and under the control of a single promoter such that the light chain and heavy chain are expressed from the same mRNA transcript. Alternatively, the two nucleic acids can be under the control of two separate promoters such that the light chain and heavy chain are expressed from two separate mRNA transcripts. In some embodiments, the nucleic acid encoding the anti-CEACAM5 antibody light chain or variable region is cloned into one expression vector and the nucleic acid encoding the anti-CEACAM5 antibody heavy chain or variable region is cloned into a second expression vector. In such embodiments, a host cell may be co-transfected with both expression vectors to produce complete antibodies or antigen-binding fragments of the invention.

### B. Host Cells

After the vector has been constructed and the one or more nucleic acid molecules encoding the components of the antibody or antigen binding fragment thereof described herein has been inserted into the proper site(s) of the vector or vectors, the completed vector(s) may be inserted into a suitable host cell for amplification and/or polypeptide expression.

Thus, in another aspect, host cells comprising nucleic acid molecules or vectors such as described herein are also provided. In various embodiments, antibody heavy chains and/or antilight chains can be expressed in prokaryotic cells, such as bacterial cells, or in eukaryotic cells, such as fungal cells (such as yeast), plant cells, insect cells, and mammalian cells. The selection of an appropriate host cell depends upon various factors, such as desired expression levels, polypeptide modifications that are desirable or necessary for activity (such as glycosylation or phosphorylation) and ease of folding into a biologically active molecule.

Introduction of one or more nucleic acids into a desired host cell can be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Exemplary prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces.

Yeast can also be used as host cells including, but not limited to, S. cerevisae, S. pombe; or K. lactis.

A variety of mammalian cell lines can be used as hosts and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TM cells (Mather et al., Annals N.Y Acad. Sci. 383: 44-68, 1982); MRC 5 cells or FS4 cells; mammalian myeloma cells, and a number of other cell lines.

Once a suitable host cell has been prepared, it can be used to express the desired antibody or antigen binding fragment thereof. Thus, in a further aspect, methods for producing an antibody or antigen binding fragment thereof as described herein are also provided. In general, such methods comprise culturing a host cell comprising one or more expression vectors as described herein in a culture medium under conditions permitting expression of the antibody or antigen binding fragment thereof as encoded by the one or more expression vectors; and recovering the antibody or antigen binding fragment thereof from the culture medium.

In some embodiments, the antibody or antigen binding fragment thereof is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### V. Therapeutic Applications

### A. Methods of Treating Diseases

In another aspect, methods of treating disorders associated with cells that express CEACAM5, e.g., cancers, are provided. In certain exemplary embodiments, the method comprises treating cancer in a cell, tissue, organ, animal or patient. Most typically, the treatment method comprises treating a cancer in a human.

“RECIST 1.1 Response Criteria” as used herein means the definitions set forth in Eisenhauer et al., Eur. J Cancer 45:228-247 (2009) for target lesions or non-target lesions, as appropriate, based on the context in which response is being measured.

The effective amount of the ADC can be administered in one or more administrations, applications or dosages and is not intended to be limited to a particular formulation or administration route.

The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment.

The frequency of administration depends on the half-life of the antibody or ADC in the circulation, the condition of the patient and the route of administration among other factors. The frequency can be, for example, daily, weekly, monthly, quarterly, or at irregular intervals in response to changes in the patient's condition or progression of the cancer being treated. An exemplary frequency for intravenous administration is between twice a week and quarterly over a continuous course of treatment, although more or less frequent dosing is also possible. Other exemplary frequencies for intravenous administration are weekly, every other week, three out of every four weeks, or every three weeks, over a continuous course of treatment, although more or less frequent dosing is also possible. For subcutaneous administration, an exemplary dosing frequency is daily to monthly, although more or less frequent dosing is also possible.

In some embodiments, provided herein is a method of treating a tumor that expresses a high level, moderate level, or any level of CEACAM5 comprising administering an ADC provided herein to a subject. In some embodiments, the level of CEACAM5 is determined by immunohistochemistry. In some embodiments, the level of CEACAM5 is determined by immunohistochemical staining of a tumor or pathology slide. In some embodiments, the level of CEACAM5 is determined by immunohistochemical staining using an antibody that binds to CEACAM5. In some embodiments, a tumor that expresses a high level of CEACAM5 is one in which at least 50% of tumor cells in a sample of the tumor score a greater than 2+ intensity as measured by immunohistochemistry. In some embodiments, at least 40%, at least 45%, at least 50%, at least 55%, at least 60%, at least 65%, at least 75%, at least 80%, at least 85% or at least 90% of cells in the sample score a greater than 2+ intensity for CEACAM5 expression as measured by immunohistochemistry In some embodiments, the sample is from a biopsy. In some embodiments, the method further comprises determining the level of CEACAM5 in a tumor sample prior to administering an ADC provided herein. In some embodiments, the ADC is administered if the tumor expresses a high level of CEACAM5.

In some embodiments, the tumor to be treated expresses a moderate level of CEACAM5. In some embodiments, a tumor that expresses a moderate level of CEACAM5 is one in which at least 1% and less than 50% of tumor cells in a sample of the tumor score at least 2+ intensity as measured by immunohistochemistry. In some embodiments, at least 2%, at least 5%, at least 10%, at least 15%, at least 20%, at least 25%, at least 30%, at least 35%, at least 40%, or at least 45% but less than 50% of cells in the sample score at least a 2+ intensity for CEACAM5 expression as measured by immunohistochemistry. In some embodiments, 5% to 45%, 10% to 40%, 15% to 35%, or 20% to 40% of cells in the sample score at least a 2+ intensity for CEACAM5 expression as measured by immunohistochemistry. In some embodiments, at least 50% of tumoral cells expressing the target at 1+ intensity or at least 1% and less than 50% of tumor cells in a sample of the tumor score at least 2+ intensity as measured by immunohistochemistry CEACAM5 expression as measured by immunohistochemistry. In some embodiments, the sample is from a biopsy. In some embodiments, the method further comprises determining the level of CEACAM5 in a tumor sample prior to administering an ADC provided herein. In some embodiments, the ADC is administered if the tumor expresses a moderate level of CEACAM5.

In some embodiments, the tumor to be treated expresses any level of CEACAM5. In some embodiments, a tumor that expresses any level of CEACAM5 is one in which reactivity for CEACAM5 is observed as measured by immunohistochemistry but that is not considered as having a moderate or high CEACAM5 expression level. In some embodiments, at least 1%, 2%, 5%, 10%, 40%, at least 45%, at least 50%, at least 55%, at least 60%, at least 65%, at least 75%, at least 80%, at least 85% or at least 90% of cells in the sample score at least a 1+ intensity as measured by immunohistochemistry. In some embodiments, the sample is from a biopsy. In some embodiments, the method further comprises determining the level of CEACAM5 in a tumor sample prior to administering an ADC provided herein. In some embodiments, the ADC is administered if the tumor expresses any level of CEACAM5.

In some embodiments, the ADC provided herein do not result in a significant level of toxicity when administered to a subject. In some embodiments, the ADC does not cause one or more side effects or toxicities typically associated with treatment with an ADC targeting CEACAM5.

In some embodiments, administering the ADCs provided herein result in a decreased tumor volume or tumor size in a subject. In some embodiments, the decreased tumor volume or size is measured using MRI, PET, CT, calipers, or ultrasound. In some embodiments, the tumor volume is reduced significantly compared to a control subject that does not receive treatment with the ADC. In some embodiments, response to the ADC is determined by comparing tumor volume change at time t to its baseline with ΔRTV=(Vt−V0)/V0×100.

In some embodiments, the cancer is a CEACAM5 expressing cancer.

Exemplary cancers suitable for treatment with the antigen binding proteins provided herein are those that express a high or moderate level of CEACAM5. Examples of cancers that can be treated with an ADC, but are not limited to solid tumors. In some embodiments, the cancer is selected from the group consisting of colorectal cancer, neuroendocrine cancers, stomach cancers, lung cancers, uterus cancers, cervical cancers, pancreatic cancers, esophagus cancers, ovarian cancers, thyroid cancers, bladder cancers, endometrium cancers, bladder cancers, endometrial cancers, breast cancers, liver cancers, prostate cancers, gastric cancers, cholangiocarcinoma and skin cancer.

Exemplary solid tumors that can be treated include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell lung carcinoma (SCLC) or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer.

In certain embodiments, the cancer is a solid tumor that is associated with ascites. Ascites is a symptom of many types of cancer and can also be caused by a number of conditions, such as advanced liver disease. The types of cancer that are likely to cause ascites include, but are not limited to, cancer of the breast, lung, large bowel (colon), stomach, pancreas, ovary, uterus (endometrium), peritoneum and the like. In some embodiments, the solid tumor associated with ascites is selected from breast cancer, colon cancer, pancreatic cancer, stomach, uterine cancer, and ovarian cancer. In some embodiments, the cancer is associated with pleural effusions, e.g., lung cancer.

In particular embodiments, the cancer is selected from the group consisting of neuroendocrine cancer, colorectal cancer, lung cancers, gastric cancers, and pancreatic cancers.

In some embodiments, the cancer is selected from the group consisting of colorectal cancer, stomach cancers, gastric cancer, Gastroesophageal Junction cancer, lung cancers, uterus cancers, cervical cancers, pancreatic cancers, esophagus cancers, ovarian cancers, thyroid cancers, bladder cancers, endometrium cancers, bladder cancers, neuroendocrine cancers, endometrial cancers, breast cancers, liver cancers, prostate cancers, and cholangiocarcinoma and skin cancers. In some embodiments, the lung cancers include Non-Small-Cell-Lung Carcinoma (NSCLC), non-squamous-NSCLC (nsq-NSCLC), squamous-NSCLC (sq-NSCLC), or Small-Cell-Lung-Carcinoma (SCLC)), or any combination thereof. In some embodiments, the pancreatic cancers include Pancreatic Ductal Adenocarcinoma (PDAC).

In some embodiments, cancer is selected from the group consisting of colorectal cancer, lung cancers, gastric cancers, Gastroesophageal Junction cancers, neuro endocrine cancers and pancreatic cancers.

In some embodiments, the cancer is colorectal cancer, NSCLC, SCLC, gastric cancers, gastroesophageal Junction cancers or Pancreatic Ductal Adenocarcinoma.

In some embodiments, the cancer is primary, metastatic or carcinosis.

In some embodiments, the method described herein causes a reduction in tumor volume following administration, such as a reduction in the tumor volume of at least about any of 5%, 10%, 15%, 20%, 25%, 30%, 35%, 40%, 45%, 50%, 55%, 60%, 65%, 70%, 75%, 80%, 85%, 90%, 95%, 99%, or 100%.

In some embodiments, the change in tumor volume for each treated (T) and control (C) may be calculated for each tumor by subtracting the tumor volume on the day of randomization (staging day) from the tumor volume on the specified observation day. The median ΔT may be calculated for the treated group and the median ΔC may be calculated for the control group. In some embodiments, the ratio ΔT/ΔC may be calculated and expressed as a percentage:

ΔT/ΔC=(delta T/delta C)×100

In some embodiments, the method described herein causes a change in tumor volume (e.g., ratio ΔT/ΔC), such as a change in tumor volume (e.g., ratio ΔT/ΔC) of less than about any of 50%, 45%, 40%, 35%, 30%, 25%, 20%, 15%1, 0%, 5%, 1%, or 0%. In some embodiments, the change in tumor volume (e.g., ratio ΔT/ΔC) of less than 0.

In some embodiments, % tumor regression may be defined as the % of tumor volume decrease in the treated group at a specified observation day compared to its volume on the day of randomization. In some embodiments, at a specific time point and for each animal, % regression can be calculated, and the median % regression may be calculated as:

$$\% regression ( at t ) = \frac{{volume}_{t 0} - {volume}_{t}}{{volume}_{t 0}} \times 100$$

In some embodiments, the method described herein causes a % tumor regression of at least about any of 5%, 10%, 15%, 20%, 25%, 30%, 35%, 40%, 45%, 50%, 55%, 60%, 65%, 70%, 75%, 80%, 85%, 90%, 95%, 99%, or 100%.

In some embodiments, the method described herein does not induce a significant level of toxicity in the individual being treated. In some embodiments, the individual does not experience an adverse event associated with the administration of the ADC provided herein. In some embodiments, the individual does not experience a severe adverse event associated with the administration of the ADC provided herein.

In some embodiments, the administration of the antibody-drug conjugate results in a strong bystander effect. In some embodiments, the bystander effect allows the payload to diffuse from antigen-positive tumor cells to adjacent antigen-negative tumor cells, resulting in cell killing. In some embodiments, the administration of the antibody-drug conjugate results in a low off-target effect.

In some embodiments, the subject has relapsed, refractory, or progressive disease. In some embodiments, the subject has no appropriate standard therapy available at the time of enrollment. In some embodiments, the subject has one of the following tumor types: Colorectal cancer (CRC), Gastric carcinoma (GC) (including signet-ring cell histology) and gastroesophageal junction adenocarcinoma (GEJ), Small cell lung cancer (SCLC), Non-small cell lung cancer (NSCLC), squamous or non-squamous histology, and Pancreatic ductal adenocarcinoma (PDAC). In some embodiments, the subject has histologically- or cytologically-confirmed metastatic or unresectable solid tumor malignancy.

In some embodiments, the subject has received one or more prior treatments. In some embodiments, the prior treatment is a standard of care treatment (SoC) for the indication. In some embodiments, the individual has received a pre-treatment before being treated with the antibody-drug conjugate or salt thereof provided herein. In some embodiments, the pre-treatment is a chemotherapy or immunotherapy. In some embodiments, the pre-treatment is selected from anti-metabolite, anti-neoplastic, alkylating agent and pro-drug agents. In some embodiments, the individual has received a pre-treatment before being treated with the antibody-drug conjugate or salt thereof or the pharmaceutical composition described herein. In some embodiments, the pre-treatment is a chemotherapy or immunotherapy. In some embodiments, the pre-treatment is selected from anti-metabolite, anti-neoplastic, alkylating agent and pro-drug agents. In some embodiments, the pre-treatment is selected from platinum-based therapy, fluoropyrimidine, oxaliplatin, irinotecan or immune checkpoint inhibitors (such as anti-PD1/PDL1 inhibitors).

In some embodiments, the subject has CRC, and has received prior treatment (in 1 or more lines of therapy) containing fluoropyrimidine, oxaliplatin, and irinotecan. In some embodiments, the subject has PDAC, and has received 1 prior line of therapy and received no more than 3 prior lines of therapy in the advanced or metastatic setting. In some embodiments, the subject has GC and/or GEJ, and has received prior platinum and fluoropyrimidine-based chemotherapy. In some embodiments, the subject has NSCLC (including non-squamous and squamous), and has received platinum-based therapy. In some embodiments, the subject is eligible and consistent with local standard of care and has received a PD-1/PD-L1 inhibitor. In some embodiments, the subject has small cell lung cancer (SCLC), and has received platinum-based therapy for extensive-stage disease and no more than 3 prior lines of therapy.

In some embodiments, the subject has a tumor site that is accessible for biopsy(ies) and agree to biopsy(ies) and/or submission of archival tissue. In some embodiments, the subject has an Eastern Cooperative Oncology Group (ECOG) Performance Status score of 0 or 1. In some embodiments, the subject has a measurable disease per Response Evaluation in Solid Tumors (RECIST) v1.1 at baseline.

In some embodiments, the subject does not have previous exposure to CEACAM5-targeted therapy. In some embodiments, the subject does not have prior treatment with an antibody-drug conjugate (ADC) with a camptothecin payload. In some embodiments, the subject does not have history of another malignancy within 3 years before the first dose of study intervention, or any evidence of residual disease from a previously diagnosed malignancy. In some embodiments, the subject does not have active cerebral/meningeal disease related to the underlying malignancy. In some embodiments, the subject has a history of cerebral/meningeal disease related to the underlying malignancy and the prior central nervous system disease has been treated and the subject is clinically stable (defined as not having received steroid treatment for symptoms related to cerebral/meningeal disease for at least 2 weeks prior to enrollment and with no ongoing related AEs).

### VI. Pharmaceutical Compositions and Formulations

The present invention provides ADC mixtures and pharmaceutical compositions comprising any of the ADCs described herein. The mixtures and pharmaceutical compositions comprise a plurality of conjugates. In some embodiments, each of the conjugates in the mixture or composition is identical or substantially identical, however, the distribution of drug-linkers on the ligands in the mixture or compositions may vary as well as the drug loading. For example, the conjugation technology used to conjugate drug-linkers to antibodies as the targeting agent in some embodiments results in a composition or mixture that is heterogeneous with respect to the distribution of Camptothecin Linker Compounds on the antibody (Ligand Unit) within the mixture and/or composition. In some of those embodiments, the loading of Camptothecin Linker Compounds on each of the antibody molecules in a mixture or composition of such molecules is an integer that ranges from 1 to 16.

In those embodiments, when referring to the composition as a whole, the loading of drug-linkers is a number ranging from 1 to about 16. Within the composition or mixture, there sometimes is a small percentage of unconjugated antibodies. The average number of drug-linkers per Ligand Unit in the mixture or composition (i.e., average drug-load) is an important attribute as it relates to the maximum amount of drug that can be delivered to the target cell. Typically, the average drug load is 1, 2 or about 2, 3 or about 3, 4 or about 4, 5 or about 5, 6 or about 6, 7 or about 7, 8 or about 8, 9 or about 9, 10 or about 10, 11 or about 11, 12 or about 12, 13 or about 13, 14 or about 14, 15 or about 15, 16 or about 16.

In some embodiments, the mixtures and pharmaceutical compositions comprise a plurality (i.e., population) of conjugates, however, the conjugates are identical or substantially identical and are substantially homogenous with respect to the distribution of drug-linkers on the ligand molecules within the mixture and/or composition and with respect to loading of drug-linkers on the ligand molecules within the mixture and/or composition. In some such embodiments, the loading of drug-linkers on an antibody Ligand Unit is 1-10, such as about 2, about 4, about 6, or about 8. In some embodiments, the loading of drug-linkers on the antibody Ligand Unit is about 8. Within the composition or mixture, there may also be a small percentage of unconjugated antibodies. The average drug load in such embodiments is about 2 or about 4. Typically, such compositions and mixtures result from the use of site-specific conjugation techniques and conjugation is due to an introduced cysteine residue.

The average number of Camptothecins or Camptothecin-Linker Compounds per Ligand Unit in a preparation from a conjugation reaction is typically characterized by conventional means such as mass spectrometry, ELISA assay, HPLC (e.g., HIC). In those instances, the quantitative distribution of ADCs in terms of subscript p is typically determined. In other instances, separation, purification, and characterization of homogeneous ADCs is typically achieved by conventional means such as reverse phase HPLC or electrophoresis.

In some embodiments, the compositions are pharmaceutical compositions comprising the ADCs described herein and a pharmaceutically acceptable carrier. In some of those embodiments, the pharmaceutical composition is in liquid form. In other of those embodiments, the pharmaceutical composition is a lyophilized powder.

The compositions, including pharmaceutical compositions, can be provided in purified form. As used herein, “purified” means that when isolated, the isolate contains at least 95%, and in other embodiments at least 98%, of Conjugate by weight of the isolate. Pharmaceutical compositions that comprise an ADC are also provided and can be utilized in any of the therapeutic applications disclosed herein. In certain embodiments, the pharmaceutical composition comprises a therapeutically effective amount of one or a plurality of the ADC, together with pharmaceutically acceptable diluent or carrier. In other embodiments, the pharmaceutical composition comprises a therapeutically effective amount of one or a plurality of the antigen binding proteins, a pharmaceutically acceptable diluent, carrier, solubilizer, emulsifier, preservative, and/or adjuvant. Acceptable formulation materials are nontoxic to recipients at the dosages and concentrations employed. The pharmaceutical compositions can be formulated as liquid, frozen or lyophilized compositions.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; bulking agents; chelating agents; complexing agents; fillers; carbohydrates such as monosaccharides or disaccharides; proteins; coloring, flavoring and diluting agents; emulsifying agents; hydrophilic polymers; low molecular weight polypeptides; salt-forming counterions (such as sodium); preservatives; solvents (such as glycerin, propylene glycol or polyethylene glycol); sugar alcohols; suspending agents; surfactants or wetting agents; stability enhancing agents; tonicity enhancing agents; delivery vehicles; and/or pharmaceutical adjuvants. Additional details and options for suitable agents that can be incorporated into pharmaceutical compositions are provided in, for example, Remington's Pharmaceutical Sciences, 22^{nd} Edition, (Loyd V. Allen, ed.) Pharmaceutical Press (2013); Ansel et al., Pharmaceutical Dosage Forms and Drug Delivery Systems, 7^{th} ed., Lippencott Williams and Wilkins (2004); and Kibbe et al., Handbook of Pharmaceutical Excipients, 3^{rd} ed., Pharmaceutical Press (2000).

The components of the pharmaceutical composition are selected depending upon, for example, the intended route of administration, delivery format and desired dosage. See, for example, Remington's Pharmaceutical Sciences, 22^{nd} Edition, (Loyd V. Allen, ed.) Pharmaceutical Press (2013). The compositions are selected to influence the physical state, stability, rate of in vivo release and rate of in vivo clearance of the antigen binding proteins disclosed. The primary vehicle or carrier in a pharmaceutical composition can be either aqueous or non-aqueous in nature. For example, a suitable vehicle or carrier can be water for injection or physiological saline solution. In certain embodiments, antigen binding protein compositions can be prepared for storage by mixing the selected composition having the desired degree of purity with optional formulation agents in the form of a lyophilized cake or an aqueous solution. Further, in certain embodiments, the antigen binding protein can be formulated as a lyophilizate using appropriate excipients.

Some compositions include a buffer or a pH adjusting agent. Representative buffers include, but are not limited to: organic acid salts (such as salts of citric acid, acetic acid, ascorbic acid, gluconic acid, carbonic acid, tartaric acid, succinic acid, or phthalic acid); Tris; phosphate buffers; and, in some instances, an amino acid as described below. In certain embodiments, buffers are used to maintain the composition at physiological pH or at a slightly lower pH, typically within a pH range of from about 5 to about 8.

Some compositions include a polyol. Polyols include sugars (e.g., mannitol, sucrose, trehalose, and sorbitol) and polyhydric alcohols such as, for instance, glycerol and propylene glycol, and polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Surfactants can be included in certain formulations.

In some embodiments, one or more antioxidants are included in the pharmaceutical composition. Antioxidant excipients can be used to prevent oxidative degradation of proteins.

A tonicity enhancing agent can also be included in certain formulations. Examples of such agents include alkali metal halides, preferably sodium or potassium chloride, mannitol, and sorbitol.

One or more preservatives can be included in certain formulations. Preservatives are necessary when developing multi-dose parenteral formulations that involve more than one extraction from the same container. Their primary function is to inhibit microbial growth and ensure product sterility throughout the shelf-life or term of use of the drug product.

A pharmaceutical composition is formulated to be compatible with its intended route of administration. Examples of routes of administration are intravenous (IV), intradermal, inhalation, transdermal, topical, transmucosal, and rectal administration. A preferred route of administration for an antigen binding protein (e.g, an antibody) is IV infusion. In another preferred embodiment, the preparation is administered by intramuscular or subcutaneous injection.

Formulation components suitable for parenteral administration (e.g., intravenous, subcutaneous, intraocular, intraperitoneal, intramuscular) include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and should be preserved against microorganisms. The carrier can be a solvent or dispersion medium containing, for example, water, ethanol, polyol (for example, glycerol, propylene glycol, and liquid polyethylene glycol), and suitable mixtures thereof.

Further guidance on appropriate formulations depending upon the form of delivery is provided, for example, in Remington's Pharmaceutical Sciences, 22^{nd} Edition, (Loyd V. Allen, ed.) Pharmaceutical Press (2013).

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

### VII. Kits/Articles of Manufacture

Kits containing an ADC as described herein are also provided. In one embodiment, such kits comprise one or more containers comprising an antigen binding protein (e.g, an anti-CEACAM5 antibody), or unit dosage forms and/or articles of manufacture. In some embodiments, a unit dosage is provided wherein the unit dosage contains a predetermined amount of a composition comprising an antigen binding protein, with or without one or more additional agents. In some embodiments, such a unit dosage is supplied in a single-use prefilled syringe for injection. In various embodiments, the composition contained in the unit dosage may comprise: saline; a buffer, other formulation components, and/or be formulate

d within a stable and effective pH range as described herein. Alternatively, in some embodiments, the composition is provided as a lyophilized powder that can be reconstituted upon addition of an appropriate liquid, for example, sterile water.

Some kits as provided herein further comprise instructions for use in the treatment of a disease associated with CEACAM5, such as cancer in accordance with any of the methods described herein. The kit can further comprise a description of how to select or identify an individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable. In some embodiments, the kit further comprises another therapeutic agent, such as those described above as suitable for use in combination with the antigen binding protein.

### EXEMPLARY EMBODIMENTS

Embodiment 1A. An antibody-drug conjugate that binds to CEACAM5 having the formula of

L-(Q-D)ₚ

or a salt thereof, wherein

L is a Ligand Unit comprising an antibody or antigen binding fragment thereof that binds to CEACAM5;

subscript p is an integer ranging from 1 to 16;

Q is a Linker Unit having a formula selected from the group consisting of:

—Z-A-RL-,—Z-A-RL-Y—,—Z-A-S*-RL-,—Z-A-B(S*)—RL-,

—Z-A-S*-RL-Y—, and —Z-A-B(S*)—RL-Y—;

wherein Z is a Stretcher Unit;

A is a bond or a Connector Unit;

B is a Parallel Connector Unit;

S* is a Partitioning Agent;

RL is a glycoside unit;

Y is a Spacer Unit; and

D is a Drug Unit having the formula of:

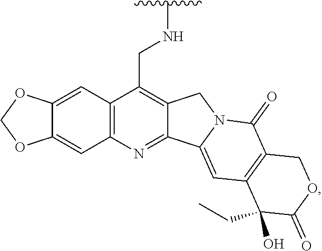 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q.

Embodiment 2A. An antibody-drug conjugate that binds to CEACAM5 having the formula of

L-(Q-D)ₚ

or a salt thereof, wherein

L is a Ligand Unit comprising an antibody or antigen binding fragment thereof that binds to CEACAM5 comprising

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8;

subscript p is an integer ranging from 1 to 16;

Q is a Linker Unit;

and D is a Drug Unit, wherein the Drug Unit is a Topoisomerase I inhibitor.

Embodiment 3A. The antibody-drug conjugate or salt thereof of embodiment 1A, wherein the antibody or antigen binding fragment thereof comprises

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8.

Embodiment 4A. The antibody-drug conjugate or salt thereof of any one of embodiments 1A-3A, wherein the antibody or antigen binding fragment thereof comprises a variable heavy chain domain (VH) that has at least 80%, 85%, 90%, 95% or 99% amino acid sequence identity to the amino acid sequence of SEQ ID NO: 7 and wherein a variable light chain domain (VL) that has at least 80%, 85%, 90%, 95% or 99% amino acid sequence identity to the amino acid sequence of SEQ ID NO: 8.

Embodiment 5A. The antibody-drug conjugate or salt thereof of any one of embodiments 1A-4A, wherein the antibody or antigen binding fragment thereof comprises

a CDR1-H comprising the amino acid sequence set forth in SEQ ID NO:1

a CDR2-H comprising the amino acid sequence set forth in SEQ ID NO:2;

a CDR3-H comprising the amino acid sequence set forth in SEQ ID NO:3;

a CDR1-L comprising the amino acid sequence set forth in SEQ ID NO:4;

a CDR2-L comprising the amino acid sequence NTR; and

a CDR3-L comprising the amino acid sequence set forth in SEQ ID NO:6.

Embodiment 6A. The antibody-drug conjugate or salt thereof of any one of embodiments 1A-5A, wherein the antibody or antigen binding fragment thereof comprises a heavy chain that has at least 80%, 85%, 90%, 95% or 99% amino acid sequence identity to the amino acid sequence set forth in SEQ ID NO:9, and a light chain that has at least 80%, 85%, 90%, 95% or 99% amino acid sequence identity to the amino acid sequence set forth in SEQ ID NO:10.

Embodiment 7A. The antibody-drug conjugate or salt thereof of any one of embodiments 1A-6A, wherein the antibody or antigen binding fragment thereof is chimeric or humanized.

Embodiment 8A. The antibody-drug conjugate or salt thereof of any one of embodiments 1A-7A, wherein the antibody or antigen binding fragment is selected from the group consisting of a of Fv, Fab, F(ab′)₂, Fab′, dsFv, (dsFv)₂, scFv, sc(Fv)₂, and a diabody.

Embodiment 9A. The antibody-drug conjugate or salt thereof of any one of embodiments 1A-8A, wherein Q is a Linker Unit having the formula —Z-A-RL-. Embodiment 10. The antibody-drug conjugate or salt thereof of any one of embodiments 1A and 3A-9A, wherein RL is a Glucuronide Unit.

Embodiment 11A. The antibody-drug conjugate or salt thereof of any one of embodiments 1A and 3A-10A, wherein RL is a Glucuronide Unit having the formula:

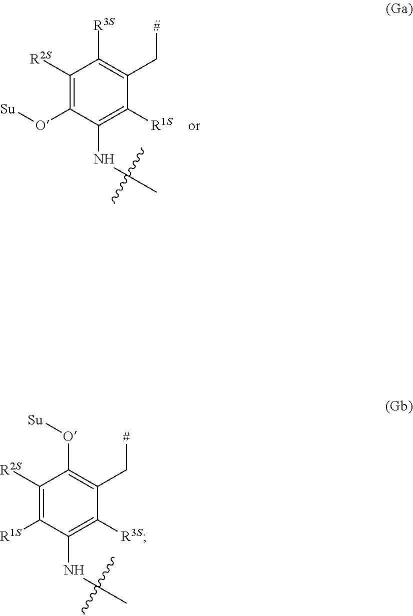

wherein

Su is a sugar moiety;

—O′— is an oxygen glycosidic bond;

R^{1S}, R^{2S} and R^{3S} independently are hydrogen, halogen, —CN, —NO₂, or other electron withdrawing group, or an electron donating group;

the wavy line indicates attachment to Z, either directly or indirectly through A or B or A and B; and

# indicates attachment to D or Y, either directly or indirectly via an intervening functional group or other moiety.

Embodiment 12A. The antibody-drug conjugate or salt thereof of any one of embodiments 1A and 3A-11A, wherein RL is a Glucuronide Unit having the formula:

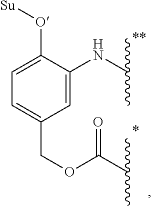

wherein

Su is a sugar moiety;

O′ represents the oxygen atom of a glycosidic bond that is capable of cleavage by a glycosidase;

the wavy line marked with a single asterisk (*) indicates the site of covalent attachment to D; and

the wavy line marked with a double asterisk (**) indicates the site of covalent attachment to the remainder of Q.

Embodiment 13A. The antibody-drug conjugate or salt thereof of embodiment 11A or 12A, wherein Su is a hexose form of a monosaccharide.

Embodiment 14A. The antibody-drug conjugate or salt thereof of any one of embodiments 1A and 3A-13A, wherein the Glucuronide Unit has the formula:

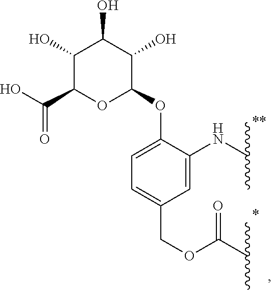 [SMILES: *Nc1cc(COC(*)=O)ccc1O[C@H]1O[C@@H](C(=O)O)[C@H](O)[C@@H](O)[C@@H]1O]

wherein

the wavy line marked with a single asterisk (*) indicates the site of covalent attachment to D; and

the wavy line marked with a double asterisk (**) indicates the site of covalent attachment to the remainder of Q.

Embodiment 15A. The antibody-drug conjugate or salt thereof of any one of embodiments 1A and 3A-14A, or a salt thereof, wherein Z comprises a succinimido-alkanoyl moiety, optionally having the succinimide ring in hydrolyzed form as a succinic acid amide moiety.

Embodiment 16A. The antibody-drug conjugate or salt thereof of any one of embodiments 1A and 3A-15A, wherein Z is

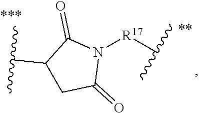

optionally having the succinimide ring in hydrolyzed form as a succinic acid amide moiety,

wherein:

the wavy line marked with a double asterisk (**) indicates the site of covalent attachment to the rest of Q;

the wavy line marked with a triple asterisk (***) indicates the point of covalent attachment to a sulfur atom of L; and

R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

Embodiment 17A. The antibody-drug conjugate or salt thereof of embodiment 16A, wherein R¹⁷ is —(CH₂)₂₋₅—C(═O)—.

Embodiment 18A. The antibody-drug conjugate or salt thereof of any one of embodiments 1A and 3A-17A, or a salt thereof, wherein Z is

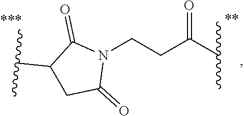 [SMILES: *C(=O)CCN1C(=O)CC(*)C1=O]

optionally having the succinimide ring in hydrolyzed form as a succinic acid amide moiety, wherein the wavy line marked with a double asterisk (**) indicates the site of covalent attachment to the rest of Q; and the wavy line marked with a triple asterisk (***) indicates the point of covalent attachment to a sulfur atom of L.

Embodiment 19A. The antibody-drug conjugate or salt thereof of any one of embodiments 1A and 3A-18A, wherein Z is

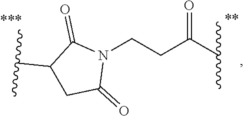 [SMILES: *C(=O)CCN1C(=O)CC(*)C1=O]

wherein the wavy line marked with a double asterisk (**) indicates the site of covalent attachment to the rest of Q; and the wavy line marked with a triple asterisk (***) indicates the point of covalent attachment to a sulfur atom of L.

Embodiment 20A. The antibody-drug conjugate or salt thereof of any one of embodiments 1A and 3A-18A, wherein Z is

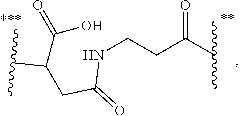 [SMILES: *C(=O)CCNC(=O)CC(*)C(=O)O]

herein the wavy line marked with a double asterisk (**) indicates the site of covalent attachment to the rest of Q; and the wavy line marked with a triple asterisk (***) indicates the point of covalent attachment to a sulfur atom of L.

Embodiment 21A. The antibody-drug conjugate or salt thereof of any one of embodiments 1A and 3A-18A, wherein Z is

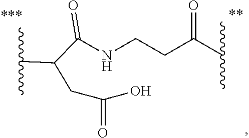 [SMILES: *C(=O)CCNC(=O)C(*)CC(=O)O]

wherein the wavy line marked with a double asterisk (**) indicates the site of covalent attachment to the rest of Q; and the wavy line marked with a triple asterisk (***) indicates the point of covalent attachment to a sulfur atom of L.

Embodiment 22A. The antibody-drug conjugate or salt thereof of any one of embodiments 1A and 3A-21A, wherein A is a Connector Unit.

Embodiment 23A. The antibody-drug conjugate or salt thereof of any one of embodiments 1A and 3A-22A, or a salt thereof, wherein A has the formula:

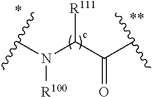

wherein

the wavy line marked with a double asterisk (**) indicates the site of covalent attachment to RL;

the wavy line marked with a single asterisk (*) indicates the point of covalent attachment to Z;

R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

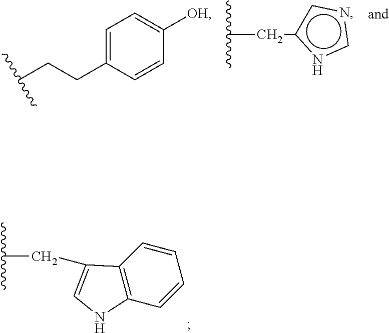

each R¹⁰⁰ is independently selected from the group consisting of hydrogen and —C₁-C₃ alkyl; and

c is an independently selected integer from 1 to 10.

Embodiment 24A. The antibody-drug conjugate or salt thereof of any one of embodiments 1A and 3A-23A, wherein A has the formula:

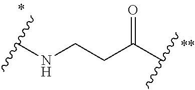 [SMILES: *NCCC(*)=O]

wherein

the wavy line marked with a double asterisk (**) indicates the site of covalent attachment to RL; and

the wavy line marked with a single asterisk (*) indicates the point of covalent attachment to Z.

Embodiment 25A. The antibody-drug conjugate or salt thereof of any one of embodiments 1A, 3A-19A, and 22A-24A, wherein —Z-A- has the formula:

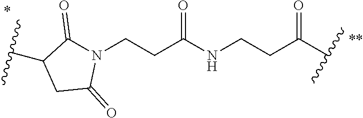 [SMILES: *C(=O)CCNC(=O)CCN1C(=O)CC(*)C1=O]

wherein

the wavy line marked with a double asterisk (**) indicates the site of covalent attachment to RL; and

the wavy line marked with a single asterisk (*) indicates the point of covalent attachment to L.

Embodiment 26A. The antibody-drug conjugate or salt thereof of any one of embodiments 1A, 3A-19A, and 22A-25A, wherein —Z-A-RL- has the formula:

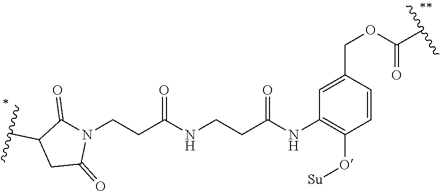

wherein

Su is a hexose form of a monosaccharide;

O′ represents the oxygen atom of a glycosidic bond that is capable of cleavage by a glycosidase;

the wavy line marked with a double asterisk (**) indicates the site of covalent attachment to D; and

the wavy line marked with a single asterisk (*) indicates the point of covalent attachment to L.

Embodiment 27A. The antibody-drug conjugate or salt thereof of any one of embodiments 1A, 3A-19A, and 22A-26A, wherein —Z-A-RL- has the formula:

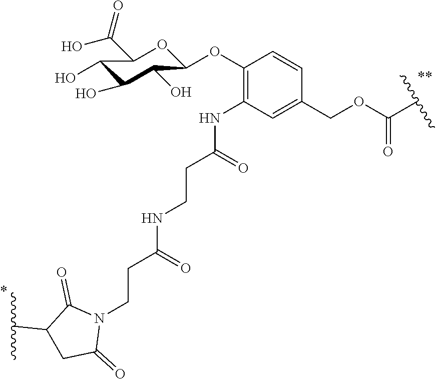

wherein

the wavy line marked with a double asterisk (**) indicates the site of covalent attachment to D; and

the wavy line marked with a single asterisk (*) indicates the point of covalent attachment to L.

Embodiment 28A. The antibody-drug conjugate or salt thereof of any one of embodiments 1A and 3A-27A, wherein —RL-D- has the formula:

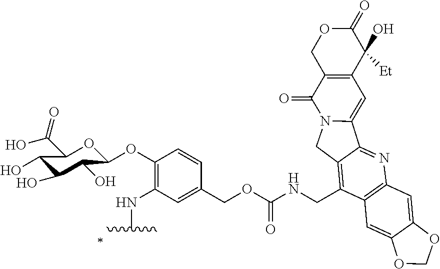

wherein

the wavy line marked with a single asterisk (*) indicates the point of covalent attachment to A.

Embodiment 29A. The antibody-drug conjugate or salt thereof of any one of embodiments 1A and 3A-28A, wherein -A-RL-D has the formula:

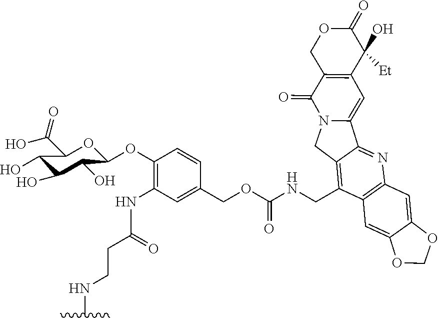

wherein

the wavy line marked with a single asterisk (*) indicates the point of covalent attachment to Z.

Embodiment 30A. The antibody-drug conjugate or salt thereof of any one of embodiments 1A, 3A-8A, and 10A-26A, or a salt thereof, wherein S* is a PEG group.

Embodiment 31A. The antibody-drug conjugate or salt thereof of any one of embodiments 1A-19A, 22A-29A, wherein -Q-D- has the formula:

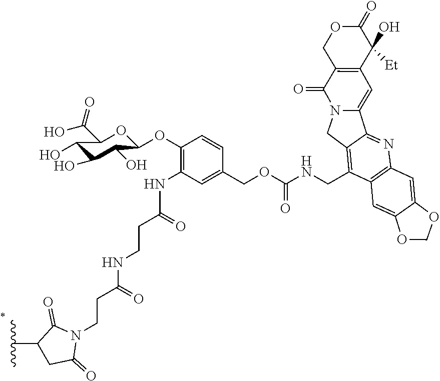

wherein

the wavy line marked with a single asterisk (*) indicates the point of covalent attachment to L.

Embodiment 32A. The antibody-drug conjugate or salt thereof of any one of embodiments 1A-18A, 20A-24A, 27A-29A, and 31A, wherein -Q-D- has the formula:

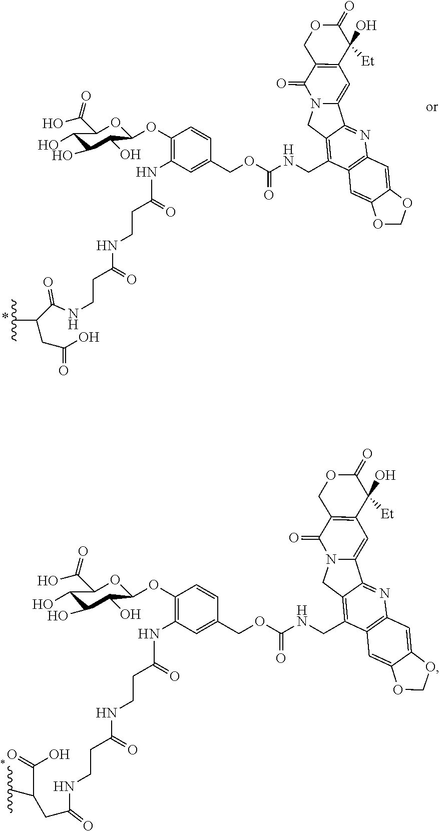

wherein

the wavy line marked with a single asterisk (*) indicates the point of covalent attachment to L.

Embodiment 33A. The antibody-drug conjugate or salt thereof of any one of embodiments 1A and 3-32A, wherein the Drug Unit is a Topoisomerase I inhibitor.

Embodiment 34A. The antibody-drug conjugate or salt thereof of any one of embodiments 1A-33A, comprising a ratio of Drug Unit to antibody (DAR) ratio of 1 to 10.

Embodiment 35A. The antibody-drug conjugate or salt thereof of any one of embodiments 1A-34A, wherein the DAR is about 4 or about 8.

Embodiment 36A. The antibody-drug conjugate or salt thereof of any one of embodiments 1A-34A, wherein p is an integer of about 1 to about 10.

Embodiment 37A. The antibody-drug conjugate or salt thereof of any one of embodiments 1A-34A, wherein p is an integer of about 4 or about 8.

Embodiment 38A. The antibody-drug conjugate or salt thereof of any one of embodiments 1A-22A, wherein the Linker Unit is attached to the antibody or antigen binding fragment at a cysteine amino acid residue.

Embodiment 39A. The antibody-drug conjugate or salt thereof of embodiment 38A, wherein the cysteine is a native cysteine.

Embodiment 40A. The antibody-drug conjugate or salt thereof of any one of embodiments 38A or 39A, wherein the cysteine is located in hinge region of the antibody or antigen-binding fragment thereof.

Embodiment 41A. The antibody-drug conjugate or salt thereof of any one of embodiments 1A-40A, wherein the antibody or antigen binding fragment thereof is cysteine engineered.

Embodiment 42A. A pharmaceutical composition comprising the antibody-drug conjugate or salt thereof of any one of embodiments 1A-41A, and a pharmaceutically acceptable carrier.

Embodiment 43A. A method of treating cancer in an individual comprising administering the antibody-drug conjugate or salt thereof of any one of embodiments 1A-41A or the pharmaceutical composition of embodiment 42A to the individual.

Embodiment 44A. The antibody-drug conjugate or salt thereof of any one of embodiments 1A-41A or the pharmaceutical composition of embodiment 42A, for use in the treatment of cancer.

Embodiment 45A. The method of embodiment 43A or the use of embodiment 44A, wherein the cancer is a solid tumor.

Embodiment 46A. The method of embodiment 43A or 45A or the use of embodiment 44A or 45A, wherein the cancer is selected from the group consisting of colorectal cancer, neuroendocrine cancer, stomach cancer, lung cancer, uterus cancer, cervical cancer, pancreatic cancer, esophagus cancer, ovarian cancer, thyroid cancer, bladder cancer, endometrium cancer, bladder cancer, endometrial cancer, breast cancer, liver cancer, prostate cancer, gastric cancer cholangiocarcinoma and skin cancer.

Embodiment 47A. The method of any one of embodiments 43A and 45A-46A or the use of any one of embodiments 44A and 45A-46A, wherein the cancer is selected from the group consisting of colorectal cancer, lung cancer, gastric cancer, and pancreatic cancer.

Embodiment 48A. The method of any one of embodiments 43A and 45A-47A or the use of any one of embodiments 44A and 45A-47A, wherein the tumor expresses a high level CEACAM5.

Embodiment 49A. The method or use of embodiment 48A, wherein at least 50% of tumor cells in a sample of the tumor score a greater than 2+ intensity as measured by immunohistochemistry.

Embodiment 50A. The method of any one of embodiments 43A and 45A-47A or the use of any one of embodiments 44A and 45A-47A, wherein the tumor expresses a moderate level CEACAM5.

Embodiment 51A. The method or use of embodiment 50A, wherein at least 1% and less than 50% of tumor cells in a sample of the tumor score a ≥2+ intensity as measured by immunohistochemistry or at least 50% of tumor cells in a sample of the tumor score a 1+ intensity as measured by immunohistochemistry.

Embodiment 52A. The method of any one of embodiments 43A and 45A-47A or the use of any one of embodiments 44A and 45A-47A, wherein the tumor expresses any level of CEACAM5.

Embodiment 53A. The method of embodiment 52A, wherein reactivity for CEACAM5 is observed but the CEACAM5 expression level is not considered moderate or high.

Embodiment 54A. The method of any one of embodiments 43A and 45A-53A or the use of any one of embodiments 44A-53A, wherein the antibody-drug conjugate or salt thereof does not induce a significant level of toxicity in the individual.

Embodiment 55A. The method of any one of embodiments 43A and 45A-54A or the use of any one of embodiments 44A-52A, wherein the antibody-drug conjugate or salt thereof causes a reduction in tumor volume following administration.

Embodiment 56A. A kit comprising the antibody-drug conjugate or salt thereof of any one of embodiments 1A-41A or the pharmaceutical composition of embodiment 42A.

### ENUMERATED EMBODIMENTS

Embodiment 1. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5;
subscript p is an integer ranging from 1 to 16;
Q is a Linker Unit having a formula selected from the group consisting of:

—Z-A-RL-,—Z-A-RL-Y—,—Z-A-S*-RL-,—Z-A-B(S*)—RL-, —Z-A-S*-RL-Y—, and —Z-A-B(S*)—RL-Y—;

wherein Z is a Stretcher Unit;
A is a bond or a Connector Unit;

### B is a Parallel Connector Unit;

### S* is a Partitioning Agent;

RL is a glycoside unit;

### Y is a Spacer Unit; and

D is a Drug Unit having the formula of:

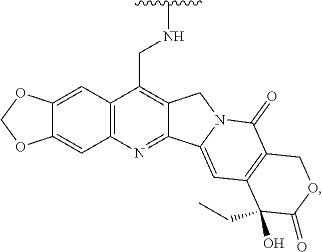 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q.

Embodiment 2. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5 comprising
a CDR1-H comprising the amino acid sequence set forth in SEQ ID NO:1
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:6;
subscript p is an integer ranging from 1 to 16;

### Q is a Linker Unit;

and D is a Drug Unit, wherein the Drug Unit is a Topoisomerase I inhibitor.

Embodiment 3. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5 comprising
a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8;
subscript p is an integer ranging from 1 to 16;

### Q is a Linker Unit;

and D is a Drug Unit, wherein the Drug Unit is a Topoisomerase I inhibitor.

Embodiment 4. The antibody-drug conjugate or salt thereof of embodiment 1 or embodiment 2, wherein the antibody or antigen binding fragment thereof comprises

a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8.

Embodiment 5. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5;
subscript p is an integer ranging from 1 to 16;
Q is a Linker Unit having a formula selected from the group consisting of:

—Z-A-RL-,—Z-A-RL-Y—,—Z-A-S*-RL-,—Z-A-B(S*)—RL-, —Z-A-S*-RL-Y—, and —Z-A-B(S*)—RL-Y—;

wherein Z is a Stretcher Unit;
A is a bond or a Connector Unit;

### B is a Parallel Connector Unit;

### S* is a Partitioning Agent;

RL is a glycoside unit;

### Y is a Spacer Unit; and

D is a Drug Unit having the formula of:

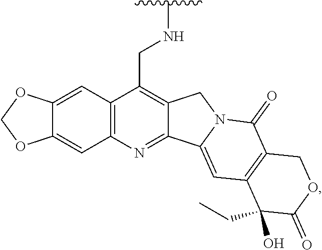 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q; and wherein the antibody or antigen binding fragment thereof comprises
a CDR1-H comprising the amino acid sequence set forth in SEQ ID NO:1
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:6.

Embodiment 6. The antibody-drug conjugate or salt thereof of any one of embodiments 1, 2, and 5, wherein the antibody or antigen binding fragment thereof comprises a variable heavy chain domain (VH) that has at least 80%, 85%, 90%, 95% or 99% amino acid sequence identity to the amino acid sequence of SEQ ID NO: 7 and a variable light chain domain (VL) that has at least 80%, 85%, 90%, 95% or 99% amino acid sequence identity to the amino acid sequence of SEQ ID NO: 8.

Embodiment 7. The antibody-drug conjugate or salt thereof of any one of embodiments 1-6, wherein the antibody or antigen binding fragment thereof comprises a heavy chain that has at least 80%, 85%, 90%, 95% or 99% amino acid sequence identity to the amino acid sequence set forth in SEQ ID NO:9, and a light chain that has at least 80%, 85%, 90%, 95% or 99% amino acid sequence identity to the amino acid sequence set forth in SEQ ID NO:10.

Embodiment 8. The antibody-drug conjugate or salt thereof of any one of embodiments 1-7, wherein the antibody or antigen binding fragment thereof comprises a heavy chain that has the amino acid sequence set forth in SEQ ID NO:9, and a light chain that has the amino acid sequence set forth in SEQ ID NO:10.

Embodiment 9. The antibody-drug conjugate or salt thereof of any one of embodiments 1-8, wherein the antibody or antigen binding fragment thereof is chimeric or humanized.

Embodiment 10. The antibody-drug conjugate or salt thereof of any one of embodiments 1-9, wherein the antibody or antigen binding fragment is selected from the group consisting of a of Fv, Fab, F(ab′)₂, Fab′, dsFv, (dsFv)₂, scFv, sc(Fv)₂, and a diabody.

Embodiment 11. The antibody-drug conjugate or salt thereof of any one of embodiments 1-10, wherein Q is a Linker Unit having the formula —Z-A-RL-.

Embodiment 12. The antibody-drug conjugate or salt thereof of any one of embodiments 1 and 4-11, wherein RL is a Glucuronide Unit.

Embodiment 13. The antibody-drug conjugate or salt thereof of any one of embodiments 1 and 4-12, wherein RL is a Glucuronide Unit having the formula:

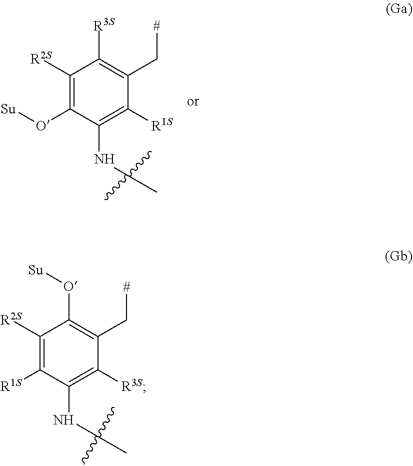

wherein
Su is a sugar moiety;
—O′— is an oxygen glycosidic bond;
R^{1S}, R^{2S} and R^{3S} independently are hydrogen, halogen, —CN, —NO₂, or other electron withdrawing group, or an electron donating group;
the wavy line indicates attachment to Z, either directly or indirectly through A or B or A and B; and
# indicates attachment to D or Y, either directly or indirectly via an intervening functional group or other moiety.

Embodiment 14. The antibody-drug conjugate or salt thereof of embodiment 13, wherein # indicates direct covalent attachment to D or Y.

Embodiment 15. The antibody-drug conjugate or salt thereof of any one of embodiments 1 and 4-14, wherein RL is a Glucuronide Unit having the formula:

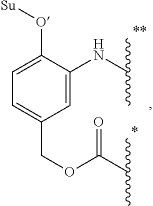

wherein
Su is a sugar moiety;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

Embodiment 16. The antibody-drug conjugate or salt thereof of any one of embodiments 13-15, wherein Su is a hexose form of a monosaccharide.

Embodiment 17. The antibody-drug conjugate or salt thereof of any one of embodiments 1 and 4-16, wherein the Glucuronide Unit has the formula:

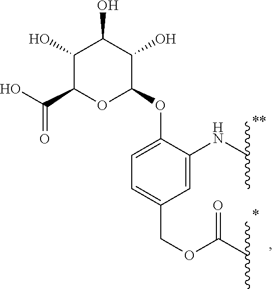 [SMILES: *Nc1cc(COC(*)=O)ccc1O[C@H]1O[C@@H](C(=O)O)[C@H](O)[C@@H](O)[C@@H]1O]

wherein
the wavy line marked with a single asterisk (*) indicates the site of covalent attachment to D; and
the wavy line marked with a double asterisk (**) indicates the site of covalent attachment to the remainder of Q.

Embodiment 18. The antibody-drug conjugate or salt thereof of any one of embodiments 1 and 4-17, or a salt thereof, wherein Z comprises a succinimido-alkanoyl moiety, optionally having the succinimide ring in hydrolyzed form as a succinic acid amide moiety.

Embodiment 19. The antibody-drug conjugate or salt thereof of any one of embodiments 1 and 4-18, wherein Z is

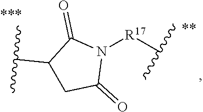

optionally having the succinimide ring in hydrolyzed form as a succinic acid amide moiety,
wherein:
the wavy line marked with a double asterisk (**) indicates the site of covalent attachment to the rest of Q;
the wavy line marked with a triple asterisk (***) indicates the point of covalent attachment to a sulfur atom of L; and
R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁—C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

Embodiment 20. The antibody-drug conjugate or salt thereof of embodiment 19, wherein R¹⁷ is —(CH₂)₂₋₅—C(═O)—.

Embodiment 21. The antibody-drug conjugate or salt thereof of any one of embodiments 1 and 4-20, or a salt thereof, wherein Z is

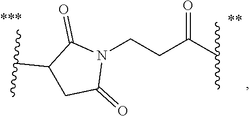 [SMILES: *C(=O)CCN1C(=O)CC(*)C1=O]

optionally having the succinimide ring in hydrolyzed form as a succinic acid amide moiety, wherein the wavy line marked with a double asterisk (**) indicates the site of covalent attachment to the rest of Q; and the wavy line marked with a triple asterisk (***) indicates the point of covalent attachment to a sulfur atom of L.

Embodiment 22. The antibody-drug conjugate or salt thereof of any one of embodiments 1 and 4-21, wherein Z is

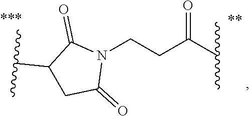 [SMILES: *C(=O)CCN1C(=O)CC(*)C1=O]

wherein the wavy line marked with a double asterisk (**) indicates the site of covalent attachment to the rest of Q; and the wavy line marked with a triple asterisk (***) indicates the point of covalent attachment to a sulfur atom of L.

Embodiment 23. The antibody-drug conjugate or salt thereof of any one of embodiments 1 and 4-21, wherein Z is

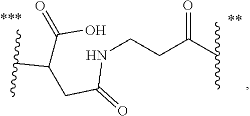 [SMILES: *C(=O)CCNC(=O)CC(*)C(=O)O]

wherein the wavy line marked with a double asterisk (**) indicates the site of covalent attachment to the rest of Q; and the wavy line marked with a triple asterisk (***) indicates the point of covalent attachment to a sulfur atom of L.

Embodiment 24. The antibody-drug conjugate or salt thereof of any one of embodiments 1 and 4-21, wherein Z is

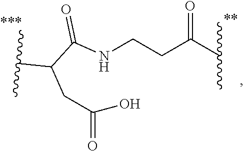 [SMILES: *C(=O)CCNC(=O)C(*)CC(=O)O]

wherein the wavy line marked with a double asterisk (**) indicates the site of covalent attachment to the rest of Q; and the wavy line marked with a triple asterisk (***) indicates the point of covalent attachment to a sulfur atom of L.

Embodiment 25. The antibody-drug conjugate or salt thereof of any one of embodiments 1 and 4-24, wherein A is a Connector Unit.

Embodiment 26. The antibody-drug conjugate or salt thereof of any one of embodiments 1 and 4-24, or a salt thereof, wherein A has the formula:

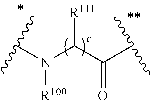

wherein

the wavy line marked with a double asterisk (**) indicates the site of covalent attachment to RL;
the wavy line marked with a single asterisk (*) indicates the point of covalent attachment to Z;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

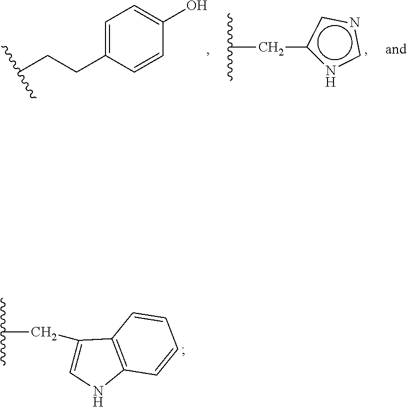

each R¹⁰⁰ is independently selected from the group consisting of hydrogen and —C₁-C₃ alkyl; and
c is an independently selected integer from 1 to 10.

Embodiment 27. The antibody-drug conjugate or salt thereof of any one of embodiments 1 and 4-26, wherein A has the formula:

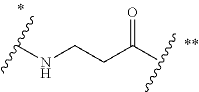

wherein

the wavy line marked with a double asterisk (**) indicates the site of covalent attachment to RL; and
the wavy line marked with a single asterisk (*) indicates the point of covalent attachment to Z.

Embodiment 28. The antibody-drug conjugate or salt thereof of any one of embodiments 1, 4-22, and 25-27, wherein —Z-A- has the formula:

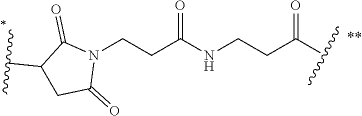

wherein
the wavy line marked with a double asterisk (**) indicates the site of covalent attachment to RL; and
the wavy line marked with a single asterisk (*) indicates the point of covalent attachment to L.

Embodiment 29. The antibody-drug conjugate or salt thereof of any one of embodiments 1, 4-22, and 25-28, wherein —Z-A-RL- has the formula:

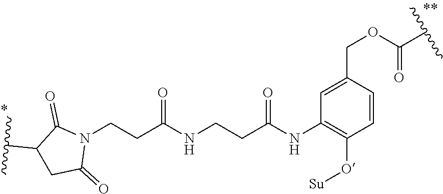

wherein
Su is a hexose form of a monosaccharide;
—O′— represents the oxygen atom of a glycosidic bond that is capable of cleavage by a glycosidase;
the wavy line marked with a double asterisk (**) indicates the site of covalent attachment to D; and
the wavy line marked with a single asterisk (*) indicates the point of covalent attachment to L.

Embodiment 30. The antibody-drug conjugate or salt thereof of any one of embodiments 1, 4-22, and 25-28, wherein —Z-A-RL- has the formula:

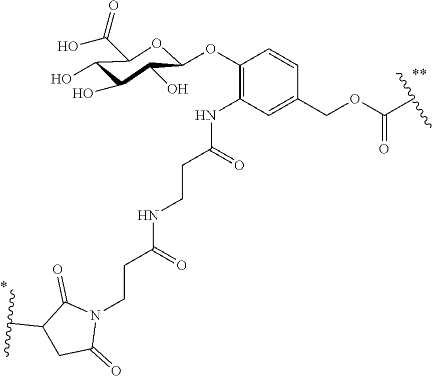

wherein
the wavy line marked with a double asterisk (**) indicates the site of covalent attachment to D; and
the wavy line marked with a single asterisk (*) indicates the point of covalent attachment to L.

Embodiment 31. The antibody-drug conjugate or salt thereof of any one of embodiments 1 and 4-30, wherein —RL-D- has the formula:

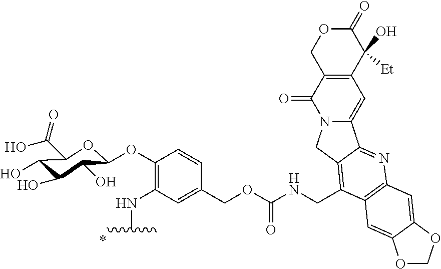

wherein
the wavy line marked with a single asterisk (*) indicates the point of covalent attachment to A.

Embodiment 32. The antibody-drug conjugate or salt thereof of any one of embodiments 1 and 4-30, wherein -A-RL-D has the formula:

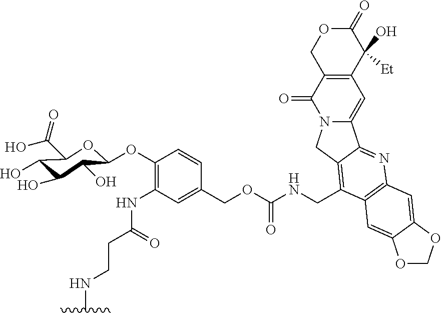

wherein
the wavy line marked with a single asterisk (*) indicates the point of covalent attachment to Z.

Embodiment 33. The antibody-drug conjugate or salt thereof of any one of embodiments 1, 4-10, and 12-29, or a salt thereof, wherein S* is a PEG group.

Embodiment 34. The antibody-drug conjugate or salt thereof of any one of embodiments 1-22, 25-32,

wherein -Q-D- has the formula:

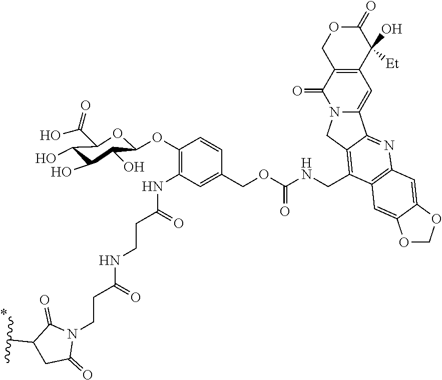

wherein
the wavy line marked with a single asterisk (*) indicates the point of covalent attachment to L.

Embodiment 35. The antibody-drug conjugate or salt thereof of any one of embodiments 1-21, 23-27, 30-32, and 34, wherein -Q-D- has the formula:

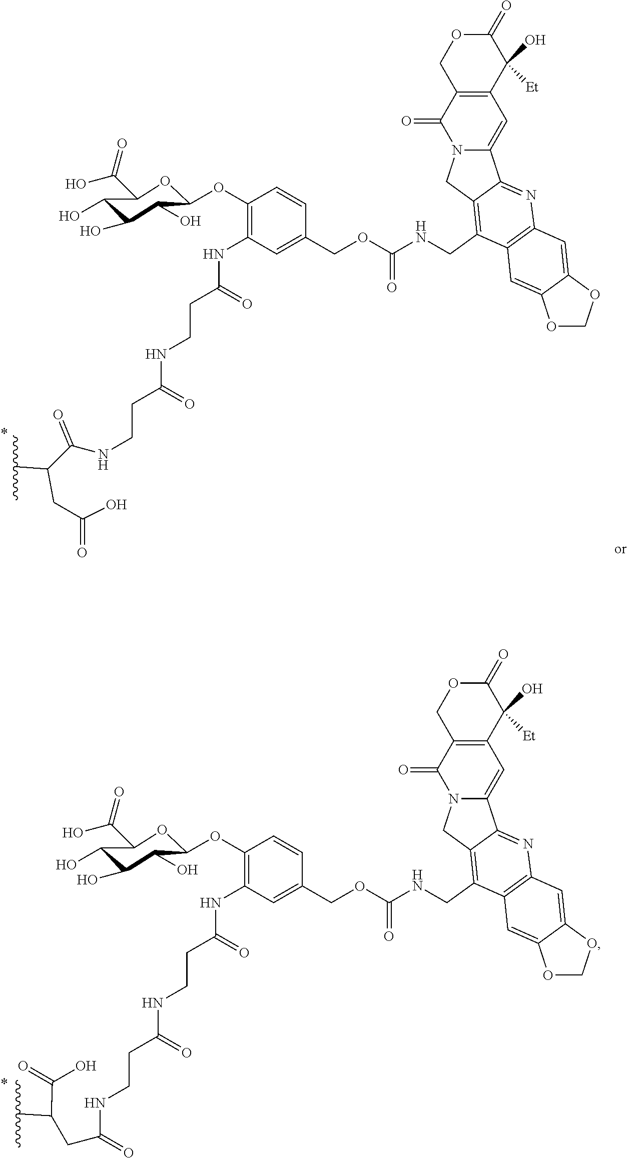

wherein
the wavy line marked with a single asterisk (*) indicates the point of covalent attachment to L.

Embodiment 36. The antibody-drug conjugate or salt thereof of any one of embodiments 1 and 4-35, wherein the Drug Unit is a Topoisomerase I inhibitor.

Embodiment 37. The antibody-drug conjugate or salt thereof of any one of embodiments 1-36, comprising a ratio of Drug Unit to antibody (DAR) ratio of 1 to 10.

Embodiment 38. The antibody-drug conjugate or salt thereof of any one of embodiments 1-37, wherein the DAR is about 4 or about 8.

Embodiment 39. The antibody-drug conjugate or salt thereof of any one of embodiments 1-37, wherein p is an integer of about 1 to about 10.

Embodiment 40. The antibody-drug conjugate or salt thereof of any one of embodiments 1-37, wherein p is an integer of about 4 or about 8.

Embodiment 41. The antibody-drug conjugate or salt thereof of any one of embodiments 1-40, wherein the Linker Unit is attached to the antibody or antigen binding fragment at a cysteine amino acid residue.

Embodiment 42. The antibody-drug conjugate or salt thereof of embodiment 41, wherein the cysteine is a native cysteine.

Embodiment 43. The antibody-drug conjugate or salt thereof of any one of embodiments 41 or 42, wherein the cysteine is located in hinge region of the antibody or antigen-binding fragment thereof.

Embodiment 44. The antibody-drug conjugate or salt thereof of any one of embodiments 1-43, wherein the antibody or antigen binding fragment thereof is cysteine engineered.

Embodiment 45. An antibody-drug conjugate or salt thereof, having the formula:

L-(Q-D)₈,

wherein L comprises an antibody or antigen binding fragment thereof that binds to CEACAM5 comprising:

a CDR1-H comprising the amino acid sequence set forth in SEQ ID NO:1

a CDR2-H comprising the amino acid sequence set forth in SEQ ID NO:2;

a CDR3-H comprising the amino acid sequence set forth in SEQ ID NO:3;

a CDR1-L comprising the amino acid sequence set forth in SEQ ID NO:4;

a CDR2-L comprising the amino acid sequence NTR; and

a CDR3-L comprising the amino acid sequence set forth in SEQ ID NO:6;

and wherein Q-D is

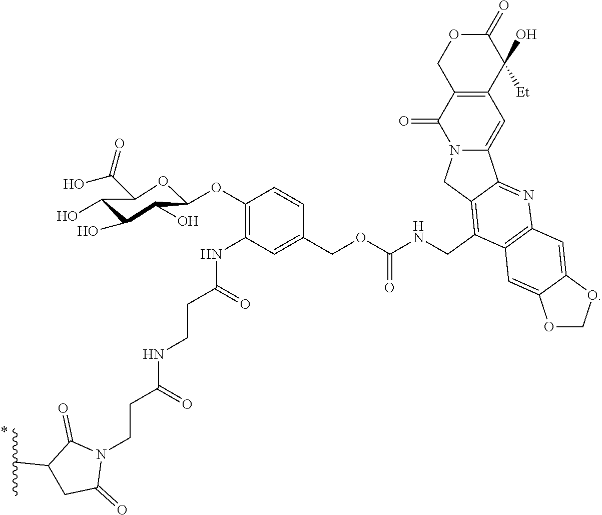

Embodiment 46. An antibody-drug conjugate or salt thereof, having the formula:

L-(Q-D)₈,

wherein L comprises an antibody or antigen binding fragment thereof that binds to CEACAM5 comprising:

a CDR1-H, a CDR2-H, and a CDR3-H of a variable heavy chain domain (VH) comprising the amino acid sequence set forth in SEQ ID NO:7 and

a CDR1-L, a CDR2-L, and a CDR3-L of a variable light chain domain (VL) comprising the amino acid sequence set forth in SEQ ID NO:8;

and wherein Q-D is

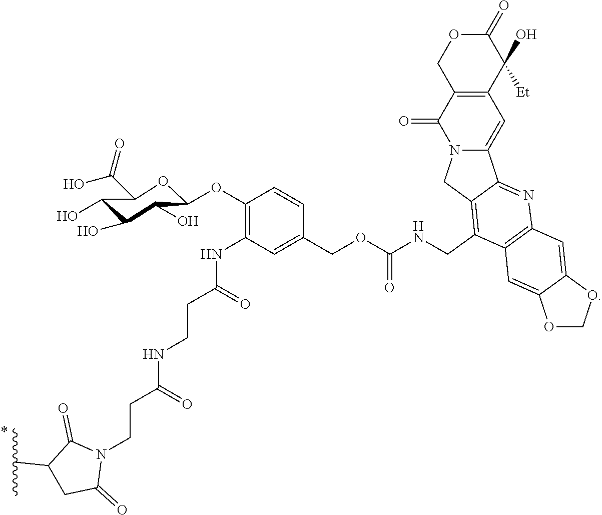

Embodiment 47. An antibody-drug conjugate or salt thereof, having the formula:

L-(Q-D)₈,

wherein L comprises an antibody or antigen binding fragment thereof that binds to CEACAM5 comprising:

a heavy chain that has the amino acid sequence set forth in SEQ ID NO:9 and

a light chain that has the amino acid sequence set forth in SEQ ID NO: 10;

and wherein Q-D is

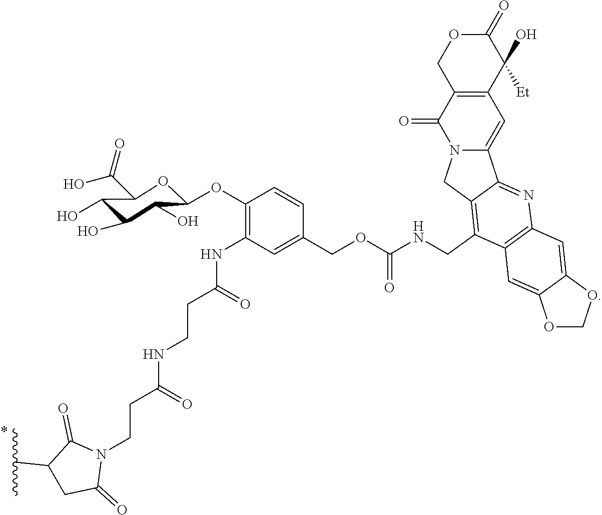

Embodiment 48. The antibody-drug conjugate of embodiment 45, wherein the antibody or antigen binding fragment thereof comprises a variable heavy chain domain (VH) comprising the amino acid sequence of SEQ ID NO: 7 and a variable light chain domain (VL) comprising the amino acid sequence of SEQ ID NO: 8

Embodiment 49. A pharmaceutical composition comprising the antibody-drug conjugate or salt thereof of any one of embodiments 1-48, and a pharmaceutically acceptable carrier.

Embodiment 50. A method of treating cancer in an individual comprising administering the antibody-drug conjugate or salt thereof of any one of embodiments 1-48 or the pharmaceutical composition of embodiment 49 to the individual.

Embodiment 51. The antibody-drug conjugate or salt thereof of any one of embodiments 1-48 or the pharmaceutical composition of embodiment 49, for use in the treatment of cancer.

Embodiment 52. The method of embodiment 50 or the use of embodiment 51, wherein the cancer is a solid tumor.

Embodiment 53. The method of embodiment 50 or 52 or the use of embodiment 51 or 52, wherein the cancer is selected from the group consisting of colorectal cancer, stomach cancers gastric cancers, Gastroesophageal Junction cancer, lung cancers, uterus cancers, cervical cancers, pancreatic cancers, esophagus cancers, ovarian cancers, thyroid cancers, bladder cancers, endometrium cancers, bladder cancers, neuroendocrine cancers, endometrial cancers, breast cancers, liver cancers, prostate cancers, and cholangiocarcinoma and skin cancers.

Embodiment 54. The method or the use of embodiment 53, wherein the individual has Non-Small-Cell-Lung Carcinoma (NSCLC), non-squamous-NSCLC (nsq-NSCLC), squamous-NSCLC (sq-NSCLC), or Small-Cell-Lung-Carcinoma (SCLC)), or any combination thereof.

Embodiment 55. The method of embodiment 50 or 52-54 or the use of any of embodiments 51-54, wherein the individual has received a pre-treatment before being treated with the antibody-drug conjugate or salt thereof of any one of embodiments 1-48 or the pharmaceutical composition of embodiment 49.

Embodiment 56. The method or use of embodiment 55, wherein the pre-treatment is a chemotherapy or immunotherapy.

Embodiment 57. The method or use of embodiment 55 or 56, wherein the pre-treatment is selected from anti-metabolite, anti-neoplastic, alkylating agent and pro-drug agents.

Embodiment 58. The method or the use of any one of embodiments 54-57, wherein the individual has small cell lung cancer (SCLC), and has received a platinum-based therapy for an extensive-stage disease and no more than 3 prior lines of therapy.

Embodiment 59. The method or the use of any one of embodiments 54-57, wherein the individual has NSCLC (including non-squamous and squamous), and has received a platinum-based therapy.

Embodiment 60. The method or use of any one of embodiments 54-57, wherein the individual has Pancreatic Ductal Adenocarcinoma (PDAC).

Embodiment 61. The method or use of embodiment 60, wherein the individual has PDAC, and has received 1 prior line of therapy and received no more than 3 prior lines of therapy in the advanced or metastatic setting.

Embodiment 62. The method or use of any one of embodiments 54-57, wherein the individual has colorectal cancer (CRC).

Embodiment 63. The method or use of embodiment 62, wherein the individual has CRC, and has received prior treatment in 1 or more lines of therapy containing fluoropyrimidine, oxaliplatin, and irinotecan.

Embodiment 64. The method or use of any one of embodiments 54-57, wherein the individual has gastric carcinoma (GC), Gastroesophageal Junction cancer (GEJ), or a combination thereof.

Embodiment 65. The method or use of embodiment 64, wherein the individual has GC, GEJ, or a combination thereof, and has received prior platinum and fluoropyrimidine-based chemotherapy.

Embodiment 66. The method of any one of embodiments 50 and 52-65, or the use of any one of embodiments 51-65, wherein the cancer is primary, metastatic or carcinosis.

Embodiment 67. The method of any one of embodiments 50 and 52-66 or the use of any one of embodiments 51-66, wherein the tumor expresses a high level CEACAM5.

Embodiment 68. The method or use of embodiment 67, wherein at least 50% of tumor cells in a sample of the tumor score a greater than 2+ intensity as measured by immunohistochemistry.

Embodiment 69. The method of any one of embodiments 50 and 52-66 or the use of any one of embodiments 51-66, wherein the tumor expresses a moderate level CEACAM5.

Embodiment 70. The method or use of embodiment 69, wherein at least 1% and less than 50% of tumor cells in a sample of the tumor score a ≥2+ intensity as measured by immunohistochemistry or at least 50% of tumor cells in a sample of the tumor score a 1+ intensity as measured by immunohistochemistry.

Embodiment 71. The method of any one of embodiments 50 and 52-66 or the use of any one of embodiments 51-66, wherein the tumor expresses any level of CEACAM5.

Embodiment 72. The method of embodiment 71, wherein reactivity for CEACAM5 is observed but the CEACAM5 expression level is not considered moderate or high.

Embodiment 73. The method of any one of embodiments 50 and 52-72, or the use of any one of embodiments 51-72, wherein the antibody-drug conjugate or salt thereof does not induce a significant level of toxicity in the individual.

Embodiment 74. The method of any one of embodiments 50 and 52-73 or the use of any one of embodiments 51-73, wherein the antibody-drug conjugate or salt thereof causes a reduction in tumor volume following administration.

Embodiment 75. The method of any one of embodiments 50 and 52-74 or the use of any one of embodiments 51-74, wherein administration of the antibody-drug conjugate results in killing of tumor cells that express CEACAM5.

Embodiment 76. The method of any one of embodiments 50 and 52-75 or the use of any one of embodiments 51-75, wherein the administration of the antibody-drug conjugate results in killing of tumor cells that express CEACAM5 and tumor cells that do not express CEACAM5.

Embodiment 77. The method of any one of embodiments 50 and 52-76, or the use of any one of embodiments 51-76, wherein the individual has relapsed, refractory, or progressive disease.

Embodiment 78. The method of any one of embodiments 50 and 52-77, or the use of any one of embodiments 51-77, wherein the individual has a histologically or cytologically-confirmed metastatic or unresectable solid tumor.

Embodiment 79. A kit comprising the antibody-drug conjugate or salt thereof of any one of embodiments 1-48 or the pharmaceutical composition of embodiment 49.

### ENUMERATED EMBODIMENTS

Embodiment 1B. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5;
subscript p is an integer ranging from 1 to 16;
Q is a Linker Unit having a formula selected from the group consisting of:

—Z-A-RL-,—Z-A-RL-Y—,—Z-A-S*-RL-,—Z-A-B(S*)—RL-, —Z-A-S*-RL-Y—, and —Z-A-B(S*)—RL-Y—;

wherein Z is a Stretcher Unit;
A is a bond or a Connector Unit;

### B is a Parallel Connector Unit;

### S* is a Partitioning Agent;

RL is a glycoside unit;

### Y is a Spacer Unit; and

D is a Drug Unit having the formula of:

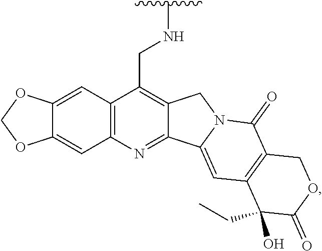 [SMILES: *NCc1c2c(nc3cc4c(cc13)OCO4)-c1cc3c(c(=O)n1C2)COC(=O)[C@]3(O)CC]

wherein the wavy line indicates the site of covalent attachment to Q.

Embodiment 2B. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5 comprising
a CDR1-H comprising the amino acid sequence set forth in SEQ ID NO:1
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:6;
subscript p is an integer ranging from 1 to 16;

### Q is a Linker Unit;

and D is a Drug Unit, wherein the Drug Unit is a Topoisomerase I inhibitor.

Embodiment 3B. An antibody-drug conjugate that binds to CEACAM5 having the formula of

L-(Q-D)ₚ

or a salt thereof, wherein
L is a Ligand Unit comprising an antibody or antigen binding fragment thereof that binds to CEACAM5 comprising
a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8;
subscript p is an integer ranging from 1 to 16;

### Q is a Linker Unit;

and D is a Drug Unit, wherein the Drug Unit is a Topoisomerase I inhibitor.

Embodiment 4B. The antibody-drug conjugate or salt thereof of embodiment 1B or embodiment 2B, wherein the antibody or antigen binding fragment thereof comprises

a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8.

Embodiment 5B. The antibody-drug conjugate or salt thereof of any one of embodiments 1B-4B, wherein the antibody or antigen binding fragment thereof comprises a heavy chain that has at least 80%, 85%, 90%, 95% or 99% amino acid sequence identity to the amino acid sequence set forth in SEQ ID NO:9, and a light chain that has at least 80%, 85%, 90%, 95% or 99% amino acid sequence identity to the amino acid sequence set forth in SEQ ID NO:10.

Embodiment 6B. The antibody-drug conjugate or salt thereof of any one of embodiments 1B-5B, wherein the antibody or antigen binding fragment thereof comprises a heavy chain that has the amino acid sequence set forth in SEQ ID NO:9, and a light chain that has the amino acid sequence set forth in SEQ ID NO:10.

Embodiment 7B. The antibody-drug conjugate or salt thereof of any one of embodiments 1B-6B, wherein the antibody or antigen binding fragment thereof is chimeric or humanized.

Embodiment 8B. The antibody-drug conjugate or salt thereof of any one of embodiments 1B-7B, wherein the antibody or antigen binding fragment is selected from the group consisting of a of Fv, Fab, F(ab′)₂, Fab′, dsFv, (dsFv)₂, scFv, sc(Fv)₂, and a diabody.

Embodiment 9B. The antibody-drug conjugate or salt thereof of any one of embodiments 1B-8B, wherein Q is a Linker Unit having the formula —Z-A-RL-.

Embodiment 10B. The antibody-drug conjugate or salt thereof of any one of embodiments 1B and 4B-9B, wherein RL is a Glucuronide Unit having the formula:

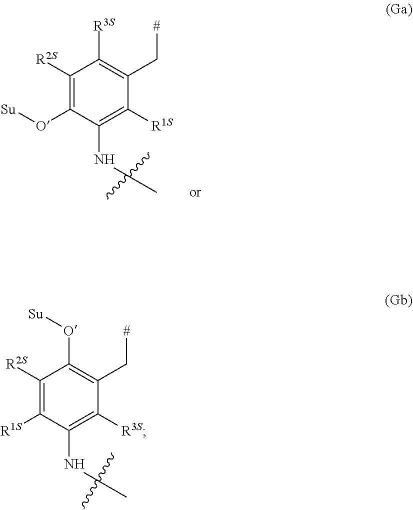

wherein
Su is a sugar moiety;
—O′— is an oxygen glycosidic bond;
R^{1S}, R^{2S} and R^{3S} independently are hydrogen, halogen, —CN, —NO₂, or other electron withdrawing group, or an electron donating group;
the wavy line indicates attachment to Z, either directly or indirectly through A or B or A and B; and
# indicates attachment to D or Y, either directly or indirectly via an intervening functional group or other moiety.

Embodiment 11B. The antibody-drug conjugate or salt thereof of embodiment 10B, wherein # indicates direct covalent attachment to D or Y.

Embodiment 12B. The antibody-drug conjugate or salt thereof of any one of embodiments 1B and 4B-11B, wherein RL is a Glucuronide Unit having the formula:

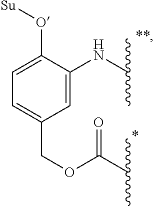

wherein
Su is a sugar moiety;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

Embodiment 13B. The antibody-drug conjugate or salt thereof of any one of embodiments 12B, wherein the Glucuronide Unit has the formula:

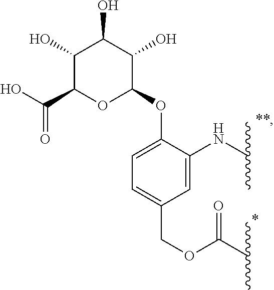 [SMILES: *Nc1cc(COC(*)=O)ccc1O[C@H]1O[C@@H](C(=O)O)[C@H](O)[C@@H](O)[C@@H]1O]

wherein
the wavy line marked with a single asterisk (*) indicates the site of covalent attachment to D; and
the wavy line marked with a double asterisk (**) indicates the site of covalent attachment to the remainder of Q.

Embodiment 14B. The antibody-drug conjugate or salt thereof of any one of embodiments 1B and 4B-13B, or a salt thereof, wherein Z comprises a succinimido-alkanoyl moiety, optionally having the succinimide ring in hydrolyzed form as a succinic acid amide moiety.

Embodiment 15B. The antibody-drug conjugate or salt thereof of any one of embodiments 1B and 4B-14B, wherein Z is

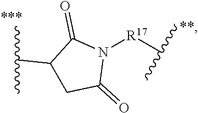

optionally having the succinimide ring in hydrolyzed form as a succinic acid amide moiety,
wherein:
the wavy line marked with a double asterisk (**) indicates the site of covalent attachment to the rest of Q;
the wavy line marked with a triple asterisk (***) indicates the point of covalent attachment to a sulfur atom of L; and
R¹⁷ is —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁—C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—.

Embodiment 16B. The antibody-drug conjugate or salt thereof of embodiment 15B, wherein R¹⁷ is —(CH₂)₂₋₅—C(═O)—.

Embodiment 17B. The antibody-drug conjugate or salt thereof of any one of embodiments 1B and 4B-16B, or a salt thereof, wherein Z is

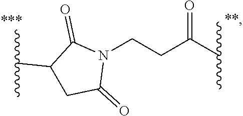 [SMILES: *C(=O)CCN1C(=O)CC(*)C1=O]

optionally having the succinimide ring in hydrolyzed form as a succinic acid amide moiety, wherein the wavy line marked with a double asterisk (**) indicates the site of covalent attachment to the rest of Q; and the wavy line marked with a triple asterisk (***) indicates the point of covalent attachment to a sulfur atom of L.

Embodiment 18B. The antibody-drug conjugate or salt thereof of any one of embodiments 1B and 4B-17B, wherein A is a Connector Unit.

Embodiment 19B. The antibody-drug conjugate or salt thereof of any one of embodiments 1B and 4B-18B, or a salt thereof, wherein A has the formula:

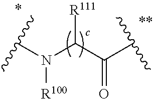

wherein

the wavy line marked with a double asterisk (**) indicates the site of covalent attachment to RL;
the wavy line marked with a single asterisk (*) indicates the point of covalent attachment to Z;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

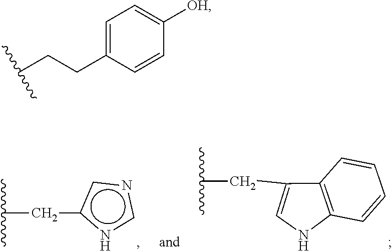

each R¹⁰⁰ is independently selected from the group consisting of hydrogen and —C₁-C₃ alkyl; and
c is an independently selected integer from 1 to 10.

Embodiment 20B. The antibody-drug conjugate or salt thereof of any one of embodiments 1B and 4B-19B, wherein A has the formula:

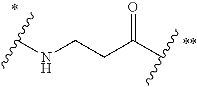 [SMILES: *NCCC(*)=O]

wherein

the wavy line marked with a double asterisk (**) indicates the site of covalent attachment to RL; and
the wavy line marked with a single asterisk (*) indicates the point of covalent attachment to Z.

Embodiment 21B. The antibody-drug conjugate or salt thereof of any one of embodiments 1B, 4B-17B, and 18B-20B, wherein —Z-A- has the formula:

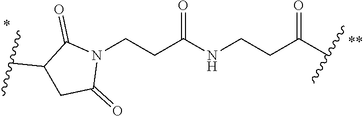 [SMILES: *C(=O)CCNC(=O)CCN1C(=O)CC(*)C1=O]

wherein
the wavy line marked with a double asterisk (**) indicates the site of covalent attachment to RL; and
the wavy line marked with a single asterisk (*) indicates the point of covalent attachment to L.

Embodiment 22B. The antibody-drug conjugate or salt thereof of any one of embodiments 1B, 4B-17B, and 18B-21B, wherein —Z-A-RL- has the formula:

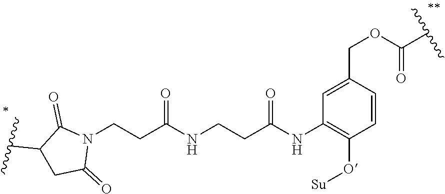

wherein
Su is a hexose form of a monosaccharide;
—O′— represents the oxygen atom of a glycosidic bond that is capable of cleavage by a glycosidase;
the wavy line marked with a double asterisk (**) indicates the site of covalent attachment to D; and
the wavy line marked with a single asterisk (*) indicates the point of covalent attachment to L.

Embodiment 23B. The antibody-drug conjugate or salt thereof of any one of embodiments 1B, 4B-17B, and 18B-22B, wherein —Z-A-RL- has the formula:

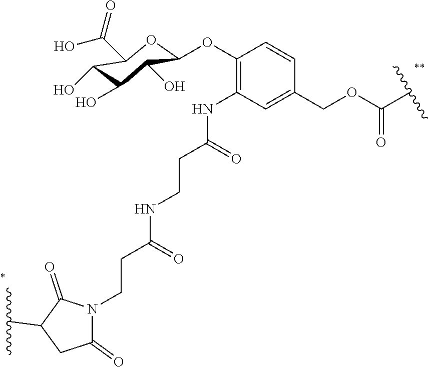

wherein
the wavy line marked with a double asterisk (**) indicates the site of covalent attachment to D; and
the wavy line marked with a single asterisk (*) indicates the point of covalent attachment to L.

Embodiment 24B. The antibody-drug conjugate or salt thereof of any one of embodiments 1B and 4B-23B, wherein —RL-D- has the formula:

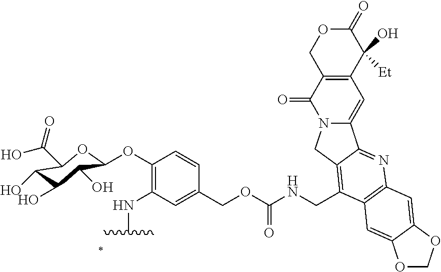

wherein
the wavy line marked with a single asterisk (*) indicates the point of covalent attachment to A.

Embodiment 25B. The antibody-drug conjugate or salt thereof of any one of embodiments 1B and 4B-24B, wherein -A-RL-D has the formula:

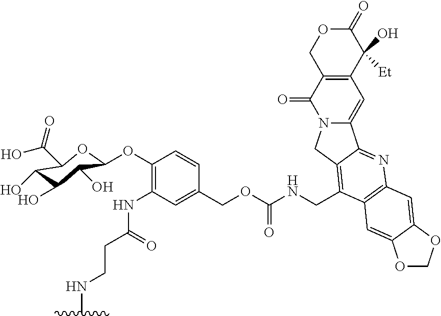

wherein
the wavy line marked with a single asterisk (*) indicates the point of covalent attachment to Z.

Embodiment 26B. The antibody-drug conjugate or salt thereof of any one of embodiments 1B, 4B-8B, and 10B-22B, or a salt thereof, wherein S* is a PEG group.

Embodiment 27B. The antibody-drug conjugate or salt thereof of any one of embodiments 1B-25B,

wherein -Q-D- has the formula:

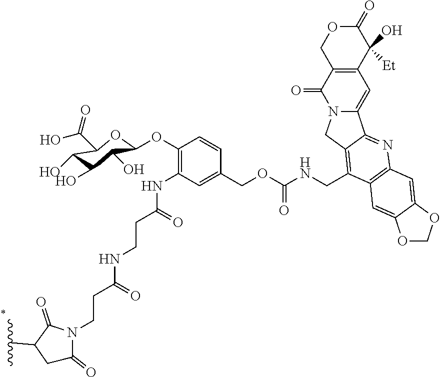

wherein
the wavy line marked with a single asterisk (*) indicates the point of covalent attachment to L.

Embodiment 28B. The antibody-drug conjugate or salt thereof of any one of embodiments 1B-20B, 23B-25B, and 27B, wherein -Q-D- has the formula:

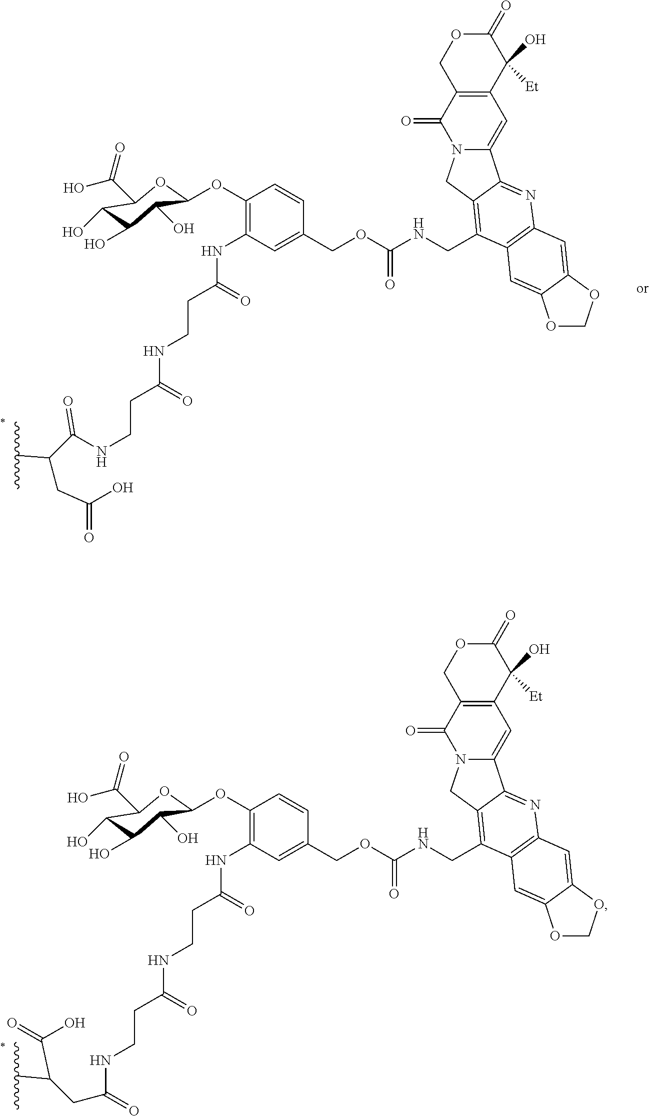

wherein
the wavy line marked with a single asterisk (*) indicates the point of covalent attachment to L.

Embodiment 29B. The antibody-drug conjugate or salt thereof of any one of embodiments 1B and 4B-28B, wherein the Drug Unit is a Topoisomerase I inhibitor.

Embodiment 30B. The antibody-drug conjugate or salt thereof of any one of embodiments 1B-29B, comprising a ratio of Drug Unit to antibody (DAR) ratio of 1 to 10.

Embodiment 31B. The antibody-drug conjugate or salt thereof of any one of embodiments 1B-30B, wherein the DAR is about 4 or about 8.

Embodiment 32. The antibody-drug conjugate or salt thereof of any one of embodiments 1B-31B, wherein the Linker Unit is attached to the antibody or antigen binding fragment at a cysteine amino acid residue.

Embodiment 33B. The antibody-drug conjugate or salt thereof of any one of embodiments 1B-31B, wherein the antibody comprises a Fc region.

Embodiment 34B. An antibody-drug conjugate or salt thereof, having the formula:

L-(Q-D)₈,

wherein L comprises an antibody or antigen binding fragment thereof that binds to CEACAM5 comprising:

a heavy chain that has the amino acid sequence set forth in SEQ ID NO:9 and

a light chain that has the amino acid sequence set forth in SEQ ID NO: 10;

and wherein Q-D is

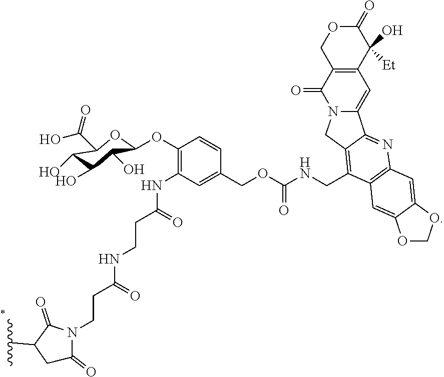

Embodiment 35B. A pharmaceutical composition comprising the antibody-drug conjugate or salt thereof of any one of embodiments 1B-34B, and a pharmaceutically acceptable carrier.

Embodiment 36B. A method of treating cancer in an individual comprising administering the antibody-drug conjugate or salt thereof of any one of embodiments 1B-34B or the pharmaceutical composition of embodiment 35B to the individual.

Embodiment 37B. The antibody-drug conjugate or salt thereof of any one of embodiments 1B-34B or the pharmaceutical composition of embodiment 35B, for use in the treatment of cancer.

Embodiment 38B. The method of embodiment 36B or the use of embodiment 37B, wherein the cancer is a CEACAM5 expressing cancer.

Embodiment 39B. The method of embodiment 36B or embodiment 38B or the use of embodiment 37B or embodiment 38B, wherein the cancer is a solid tumor.

Embodiment 40B. The method of embodiment 36B, 38B or 39B or the use of embodiment 37B-39B, wherein the cancer is selected from the group consisting of colorectal cancer, stomach cancers gastric cancers, Gastroesophageal Junction cancer, lung cancers, uterus cancers, cervical cancers, pancreatic cancers, esophagus cancers, ovarian cancers, thyroid cancers, bladder cancers, endometrium cancers, bladder cancers, neuroendocrine cancers, endometrial cancers, breast cancers, liver cancers, prostate cancers, and cholangiocarcinoma and skin cancers.

Embodiment 41B. The method or the use of embodiment 40B, wherein the individual has Non-Small-Cell-Lung Carcinoma (NSCLC), non-squamous-NSCLC (nsq-NSCLC), squamous-NSCLC (sq-NSCLC), or Small-Cell-Lung-Carcinoma (SCLC)), Pancreatic Ductal Adenocarcinoma (PDAC), colorectal cancer (CRC), gastric carcinoma (GC), Gastroesophageal Junction cancer (GEJ), or any combination thereof.

Embodiment 42B. The method of embodiment 36B or 38B-41B or the use of embodiment 37B-41B, wherein the individual has received a pre-treatment before being treated with the antibody-drug conjugate or salt thereof of any one of embodiments 1B-34B or the pharmaceutical composition of embodiment 35B.

Embodiment 43B. The method or use of embodiment 42B, wherein the pre-treatment is a chemotherapy or immunotherapy.

Embodiment 44B. The method or use of embodiment 42B or 43B, wherein the pre-treatment is selected from anti-metabolite, anti-neoplastic, alkylating agent and pro-drug agents.

Embodiment 45B. The method or use of any one of embodiments 42B-44B, wherein the pre-treatment is selected from platinum-based therapy, fluoropyrimidine, oxaliplatin, irinotecan or immune checkpoint inhibitors (such as anti-PD1/PDL1 inhibitors).

Embodiment 46B. The method of any one of embodiments 36B and 38B-45B or the use of any one of embodiments 37B-45B, wherein the tumor expresses a high level CEACAM5.

Embodiment 47B. The method of any one of embodiments 36B and 38B-46B, or the use of any one of embodiments 37B-46B, wherein the cancer is primary, metastatic or carcinosis.

Embodiment 48B. The method of any one of embodiments 36B and 38B-47B or the use of any one of embodiments 37B-47B, wherein the administration of the antibody-drug conjugate results in a strong bystander effect.

Embodiment 49B. The method of any one of embodiments 36B and 38B-48B or the use of any one of embodiments 37B-48B, wherein the administration of the antibody-drug conjugate results in a low off-target effect.

Embodiment 50B. A kit comprising the antibody-drug conjugate or salt thereof of any one of embodiments 1B-34B or the pharmaceutical composition of embodiment 35B.

### EXAMPLES

The following examples, including the experiments conducted and the results achieved, are provided for illustrative purposes only and are not to be construed as limiting the scope of the appended claims.

### Synthetic Example A—Materials and Methods

The following materials and methods are applicable to the synthetic procedures described herein unless indicated otherwise. All commercially available anhydrous solvents were used without further purification. Starting materials, reagents and solvents were purchased from commercial suppliers (SigmaAldrich and Fischer). Products were purified by flash column chromatography utilizing a Biotage Isolera One™ flash purification system (Charlotte, NC). UPLC-MS was performed on a Waters single quad detector mass spectrometer interfaced to a Waters Acquity™ UPLC system using UPLC methods shown in Tables A-F. Preparative HPLC was carried out on a Waters 2454 Binary Gradient Module solvent delivery system configured with a Wasters 2998 PDA detector. Products were purified Phenomenex Max-RP 4 μm Synergi™ 80 Å250 mm reverse phase column of appropriate diameter eluting with 0.05% trifluoroacetic acid in water and 0.05% trifluoroacetic acid in acetonitrile unless otherwise specified.

TABLE A
Column - Waters Acuity UPLC BEH C18 2.1 × 50 mm, 1.7 μm,
reversed-phase column, Solvent A - 0.1% aqueous formic acid,
Solvent B - acetonitrile with 0.1% formic acid (Method A).

| Time (min) | Flow (mL/min) | A % | B % | Gradient |
|---|---|---|---|---|
| Initial | 0.5 | 97 | 3 | |
| 1.70 | 0.5 | 40 | 60 | Linear |
| 2.00 | 0.5 | 5 | 95 | Linear |
| 2.50 | 0.5 | 5 | 95 | Linear |
| 2.80 | 0.5 | 97 | 3 | Linear |
| 3.00 | 0.5 | 97 | 3 | Linear |

TABLE B
Column - Waters Acuity UPLC BEH C18 2.1 × 50 mm, 1.7 μm,
reversed-phase column, Solvent A - 0.1% aqueous formic acid,
Solvent B - acetonitrile with 0.1% formic acid (Method B).

| Time (min) | Flow (mL/min) | A % | B % | Gradient |
|---|---|---|---|---|
| Initial | 0.6 | 97 | 3 | |
| 1.50 | 0.6 | 5 | 95 | Linear |
| 2.40 | 0.6 | 5 | 95 | Linear |
| 2.50 | 0.6 | 97 | 3 | Linear |
| 2.80 | 0.6 | 97 | 3 | Linear |

TABLE C
Column - Kinetex F5 1.7 μm 100 Å, 2.1 × 50
mm, reversed-phase column, Solvent A - 0.1% aqueous formic
acid, Solvent B - acetonitrile with 0.1% formic acid (Method C).

| Time (min) | Flow (mL/min) | A % | B % | Gradient |
|---|---|---|---|---|
| Initial | 0.5 | 97 | 3 | |
| 2.50 | 0.5 | 40 | 95 | Linear |
| 3.50 | 0.5 | 5 | 95 | Linear |
| 3.75 | 0.5 | 97 | 3 | Linear |
| 4.00 | 0.5 | 97 | 3 | Linear |

TABLE D
Column - Waters CORTECS C18 1.6 μm, 2.1 × 50 mm,
reversed-phase column, Solvent A - 0.1% aqueous formic
acid, Solvent B - acetonitrile with 0.1% formic acid (Method D).

| Time (min) | Flow (mL/min) | A % | B % | Gradient |
|---|---|---|---|---|
| Initial | 0.6 | 97 | 3 | |
| 1.70 | 0.6 | 40 | 60 | Linear |
| 2.00 | 0.6 | 5 | 95 | Linear |
| 2.50 | 0.6 | 5 | 95 | Linear |
| 2.80 | 0.6 | 97 | 3 | Linear |
| 3.00 | 0.6 | 97 | 3 | Linear |

TABLE E
Column - Waters CORTECS C18 1.6 μm, 2.1 × 50 mm,
reversed-phase column, Solvent A - 0.1% aqueous formic
acid, Solvent B - acetonitrile with 0.1% formic acid (Method E).

| Time (min) | Flow (mL/min) | A % | B % | Gradient |
|---|---|---|---|---|
| Initial | 0.6 | 97 | 3 | |
| 1.50 | 0.6 | 5 | 95 | Linear |
| 2.40 | 0.6 | 5 | 95 | Linear |
| 2.50 | 0.6 | 97 | 3 | Linear |
| 2.80 | 0.6 | 97 | 3 | Linear |

TABLE F
Column - Waters CORTECS C8 1.6 μm, 2.1 × 50 mm,
reversed-phase column, Solvent A - 0.1% aqueous formic
acid, Solvent B - acetonitrile with 0.1% formic acid (Method F).

| Time (min) | Flow (mL/min) | A % | B % | Gradient |
|---|---|---|---|---|
| Initial | 0.6 | 97 | 3 | |
| 1.50 | 0.6 | 5 | 95 | Linear |
| 2.40 | 0.6 | 5 | 95 | Linear |
| 2.50 | 0.6 | 97 | 3 | Linear |
| 2.80 | 0.6 | 97 | 3 | Linear |

TABLE G
List of Abbreviations

| | |
|---|---|
| AcOH | acetic acid |
| Boc | tert-butyloxycarbonyl protecting group |
| DCM | dichloromethane |
| DIPEA | N,N-diisopropylethylamine |
| DMA | N,N-dimethyacetamide |
| DMF | N,N-dimethylformamide |
| EtOAc | ethyl acetate |
| EtOH | ethanol |
| Fmoc | 9-fluorenylmethyl carbamates |
| HATU | 1-[bis(dimethylamino)methylene]-1H-1,2,3- |
| | triazolo[4,5-b]pyridinium 3-oxid |
| | hexafluorophosphate |
| Hex | hexanes |
| HPLC | high performance liquid chromatography |
| MeCN | acetonitrile |
| MeOH | methanol |
| MP | 3-maleimidopropyl |
| MS | Mass spectrometry |
| OSu | N-hydroxysuccinimide |
| PEG | polyethylene glycol |
| PPTS | pyridinium para-toluene sulfonic acid |
| Prep | preparative |
| TFA | trifluoroacetic acid |
| TSTU | N,N,N′,N′-tetramethyl-O-(N- |
| | succinimidyl)uronium tetrafluoroborate |
| UPLC | Ultra Performance Liquid Chromatography |

### Synthetic Example B—Camptothecin Compound Preparations

The Camptothecin Compounds provided in the following Examples can be used in preparing Camptothecin-Linker Compounds as well as Camptothecin Conjugates as described herein.

### Synthetic Example 1

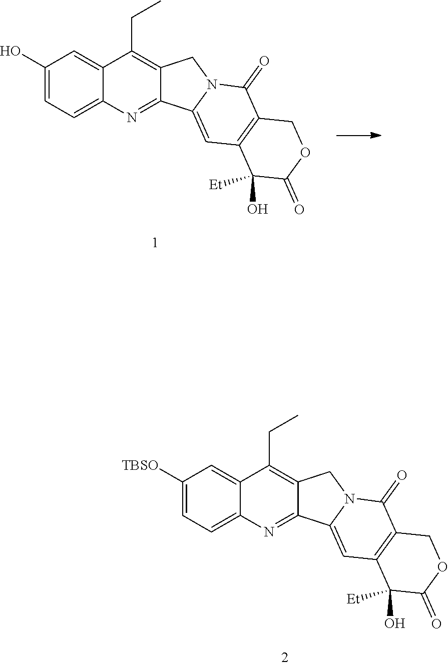 [SMILES: CCc1c2c(nc3ccc(O)cc13)-c1cc3c(c(=O)n1C2)COC(=O)[C@]3(O)CC ; CCc1c2c(nc3ccc(O[Si](C)(C)C(C)(C)C)cc13)-c1cc3c(c(=O)n1C2)COC(=O)[C@]3(O)CC]

SN-38 (compound 1, 160.0 mg, 0.4077 mmol), purchased from MedChemExpress, was suspended in anhydrous DCM (2 mL). DIPEA (0.22 mL, 1.3 mmol) was added followed by TBSCl (154 mg, 1.02 mmol). The reaction was stirred for 30 minutes until compound 1 becomes soluble and complete conversion was observed by UPLC-MS. The reaction was quenched with MeOH, filtered through plug of silica, and concentrated in vacuo. The colorless oil obtained was triturated with Hex. The product precipitated out of solution. The precipitate was collected by filtration and rinsed with Hex to afford compound 2 (TBS-SN-38) as an off-white solid (200 mg, 0.395 mmol, 97%). LC-MS (Method B): t_{R}=1.86 min; MS (m/z) [M+H]⁺ calc. for C₂₈H₃₅N₂O₅Si 507.23, found 506.96.

### Synthetic Example 2

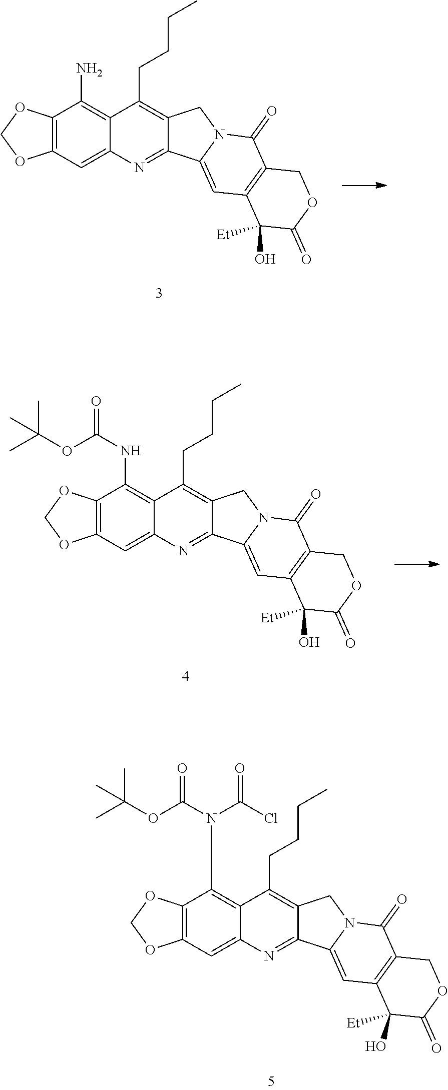 [SMILES: CCCCc1c2c(nc3cc4c(c(N)c13)OCO4)-c1cc3c(c(=O)n1C2)COC(=O)[C@]3(O)CC ; CCCCc1c2c(nc3cc4c(c(NC(=O)OC(C)(C)C)c13)OCO4)-c1cc3c(c(=O)n1C2)COC(=O)[C@]3(O)CC ; CCCCc1c2c(nc3cc4c(c(N(C(=O)Cl)C(=O)OC(C)(C)C)c13)OCO4)-c1cc3c(c(=O)n1C2)COC(=O)[C@]3(O)CC]

Compound 3 was synthesized according to the procedure described by Bioconjugate Chem. 2009, 20, 1242-1250. Compound 3 (50 mg, 0.108 mmol) dissolved in DCM (1 mL). DMAP (13 mg, 0.11 mmol) was added to the reaction followed by Boc₂O (24 mg, 0.11 mmol). The reaction was stirred for 5 minutes at which time complete conversion to the desired product was observed. The protected product was purified by column chromatography 10 G Biotage Ultra 0-5% MeOH in DCM. Fractions containing the desired product were concentrated in vacuo to afford compound 4 as a yellow solid (49 mg, 0.087 mmol, 80%). LC-MS (Method A): t_{R}=2.24 min; MS (m/z) [M+H]⁺ calc. for C₃₀H₃₄N₃O₈ 564.23, found 564.10.

Compound 4 (49 mg, 0.087 mmol) was dissolved in anhydrous DCM (2 mL). DMAP (37 mg, 0.304 mmol) was added and the reaction was cooled to 0° C. Triphosgene (12 mg, 0.039 mmol) dissolved 10 mg/mL in DCM was added dropwise to the reaction over 15 minutes. A 2 μL aliquot was quenched into 98 μL MeOH diluent and injected onto the UPLC-MS. Complete conversion to the MeOH adduct was observed by UPLC-MS. The reaction mixture (compound 5) can be used directly in coupling steps with suitable linkers. LC-MS (Method A): t_{R}=2.09 min; MS (m/z) [M+H]⁺ calc. for C₃₂H₃₆N₃O₁₀ 622.24, found 622.02.

### Synthetic Example 3

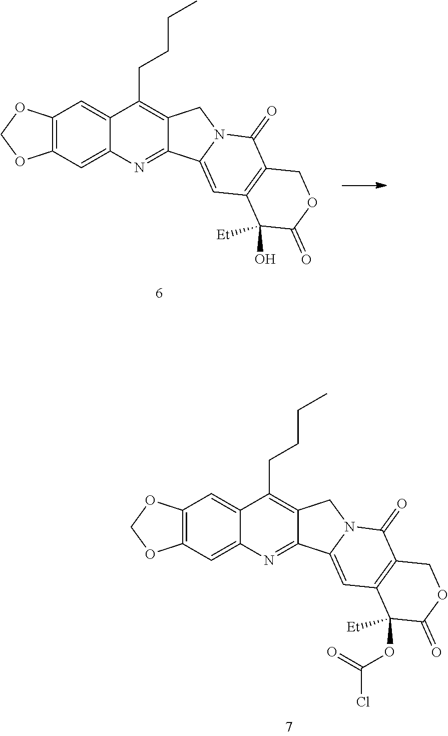 [SMILES: CCCCc1c2c(nc3cc4c(cc13)OCO4)-c1cc3c(c(=O)n1C2)COC(=O)[C@]3(O)CC ; CCCCc1c2c(nc3cc4c(cc13)OCO4)-c1cc3c(c(=O)n1C2)COC(=O)[C@@]3(CC)OC(=O)Cl]

Compound 6 (150 mg, 0.334 mmol), synthesized according to the procedure described in Bioconjugate Chem. (2009) 20: 1242-1250, was dissolved in anhydrous DCM (2 mL). DMAP (143 mg, 1.17 mmol) was added. Triphosgene (45 mg, 0.15 mmol) dissolved in anhydrous DCM (50 mg/mL) was added dropwise over 5 minutes. The reaction was stirred for 30 minutes at room temperature. A 2 μL aliquot of the reaction mixture was quenched in 98 μL MeOH diluent. Nearly complete conversion to MeOH carbonate observed indicating chloroformate formation. Compound 7 so obtained is used without further purification in coupling steps with suitable linkers. LC-MS (Method A): t_{R}=1.55 min; MS (m/z) [M+H]⁺ calc. for C₂₇H₂₇N₂O₈ 507.18, found 507.06.

### Synthetic Example 4

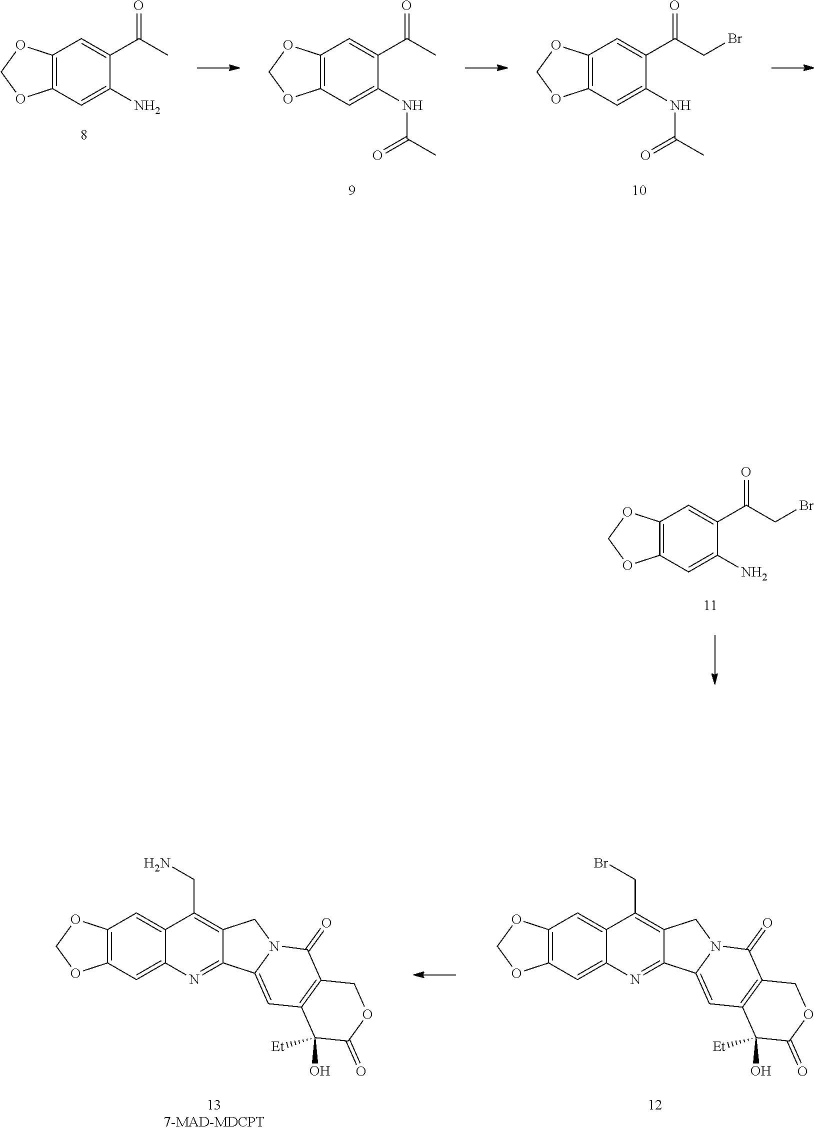 [SMILES: CC(=O)c1cc2c(cc1N)OCO2 ; CC(=O)Nc1cc2c(cc1C(C)=O)OCO2 ; CC(=O)Nc1cc2c(cc1C(=O)CBr)OCO2 ; Nc1cc2c(cc1C(=O)CBr)OCO2 ; CC[C@@]1(O)C(=O)OCc2c1cc1n(c2=O)Cc2c-1nc1cc3c(cc1c2CN)OCO3 ; CC[C@@]1(O)C(=O)OCc2c1cc1n(c2=O)Cc2c-1nc1cc3c(cc1c2CBr)OCO3]

6-Amino-3,4-(methylenedioxy)-acetophenone (8, 5.00 g, 27.9 mmol), obtained from TCI Research Chemicals (Cat. No. A1356), was dissolved in DCM (100 mL). The reaction was cooled to 0° C. and DIPEA (7.29 mL, 41.9 mmol) was added followed by slow addition of acetyl chloride (2.49 mL, 34.9 mL). The reaction was allowed to warm to room temperature and stirred for 30 minutes. Complete conversion was observed by UPLC-MS. The reaction was quenched with MeOH (5 mL), and the reaction was concentrated in vacuo to afford compound 9 as a white solid used in the next step without further purification. LC-MS (Method A): t_{R}=1.37; MS (m/z) [M+H]⁺ calc. for C₁₁H₁₂NO₄ 222.08, found 222.11.

Compound 9 (27.9 mmol) was dissolved in AcOH (100 mL). HBr 33% w/w in AcOH (9.78 mL, 55.8 mmol) was added slowly. Bromine (1.44 mL, 27.9 mmol) was added dropwise over 15 minutes. The reaction was stirred for 30 minutes at which time conversion to desired product was observed. The reaction was poured over ice water and the precipitate was collected by filtration and washed with water. The filtrate was dried to afford a yellow powder which was a mixture of the desired product compound 10 with starting material and dibrominated product impurities which was used in the next step without further purification (7.2 g, 24 mmol, 86%). LC-MS (Method A): t_{R}=1.58 min; MS (m/z) [M+H]⁺ calc. for C₁₁H₁₁BrNO₄ 299.99, found 299.90.

Compound 10 (7.2 g, 24 mmol) was dissolved in EtOH (100 mL). Concentrated HBr (5 mL) was added and the reaction was heated to reflux for 60 minutes. Nearly complete conversion to the deprotected product was observed. The reaction was concentrated in vacuo, diluted with DCM (200 mL) and H₂O (200) mL. The aqueous phase was extracted with DCM (3×200 mL), the collected organic phases were dried with MgSO₄, filtered and concentrated in vacuo. The crude product was purified by column chromatography 0-10% MeOH in DCM. Fractions containing the desired product with minor impurity were concentrated to afford compound 11 as a yellow powder (4.05 g, 15.7 mmol, 65%). LC-MS (Method A): t_{R}=1.57 min; MS (m/z) [M+H]⁺ calc. for C₉H₉BrNO₃ 257.98, found 257.71.

### Synthetic Example 5

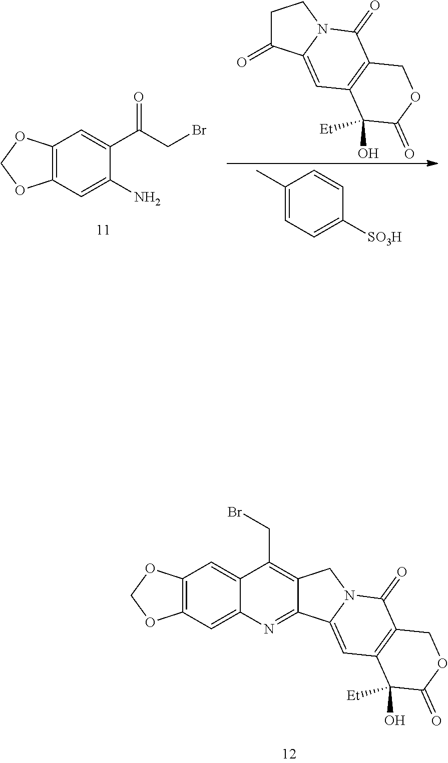

Compound 11 (1.00 g, 3.87 mmol), p-TSA (667 mg, 3.87 mmol), and 4-Ethyl-4-hydroxy-7,8-dihydro-1H-pyrano[3,4-f]indolizine-3,6,10(4H)-trione (1.02 g, 3.87 mmol, obtained from Avra Laboratories Pvt. Ltd.) were charged in a flask. DCM (5 mL) was added to homogenize the solids, and then evaporated under nitrogen. The neat solids were then heated to 120° C. under high vacuum (1 mbar) for 60 minutes. Reaction was cooled to room temperature, the crude product precipitated with H₂O, filtered and washed with H₂O. The precipitate was purified by column chromatography 0-10% MeOH in DCM. Fractions containing the desired product were concentrated in vacuo to afford compound 12 as a brown solid (989 mg, 2.04 mmol, 53%). LC-MS (Method A): t_{R}=1.62 min (General Method UPLC); MS (m/z) [M+H]⁺ calc. for C₂₂H₁₇BrN₂O₆ 485.03, found 484.95.

### Synthetic Example 6

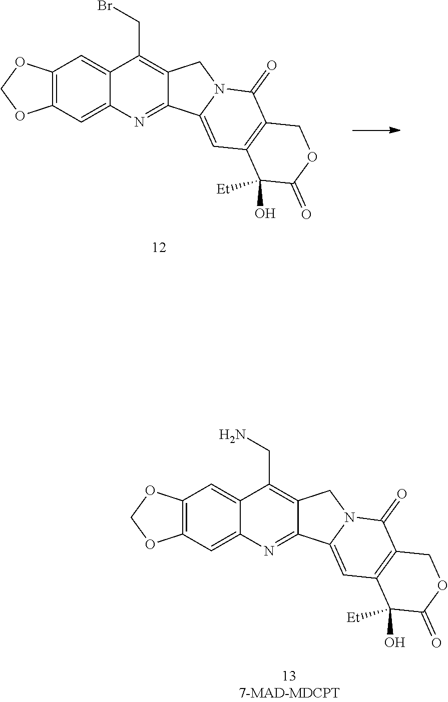 [SMILES: CC[C@@]1(O)C(=O)OCc2c1cc1n(c2=O)Cc2c-1nc1cc3c(cc1c2CBr)OCO3 ; CC[C@@]1(O)C(=O)OCc2c1cc1n(c2=O)Cc2c-1nc1cc3c(cc1c2CN)OCO3]

Compound 12 (188 mg, 0.387 mmol) was dissolved in EtOH (5 mL). Hexamethylenetetramine (163 mg, 1.16 mmol) was added and the reaction as stirred at reflux for 90 minutes. The reaction was cooled and aq. conc. HCl (0.1 mL) was added. The reaction was concentrated and purified by prep-HPLC. Fractions containing the desired product were lyophilized to afford compound 13 as an off white solid (109 mg, 0.259 mmol, 67%). LC-MS (Method A): t_{R}=0.89; MS (m/z) [M+H]⁺ calc. for C₂₂H₂₀N₃O₆ 422.14, found 422.16.

TABLE H
Camptothecin compounds prepared from compound 13 (7-MAD-MDCPT)

| | | | Calc'd MS | | |
|---|---|---|---|---|---|
| | | Parent | (m/z) | Observed | |
| No. | Structure | Exact Mass | [M + H]⁺ | MS (m/z) | t_{R} |
| 13a | 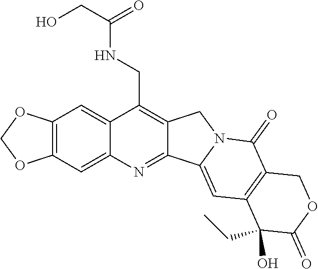 [SMILES: CC[C@@]1(O)C(=O)OCc2c1cc1n(c2=O)Cc2c-1nc1cc3c(cc1c2CNC(=O)CO)OCO3] | 479.13 | 480.14 | 480.08 | 1.20 |
| 13b | 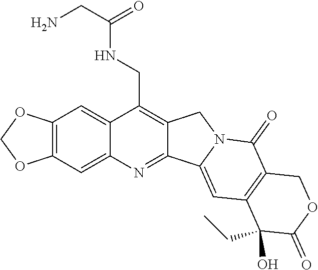 [SMILES: CC[C@@]1(O)C(=O)OCc2c1cc1n(c2=O)Cc2c-1nc1cc3c(cc1c2CNC(=O)CN)OCO3] | 478.15 | 479.16 | 479.11 | 1.05 |
| 13c | 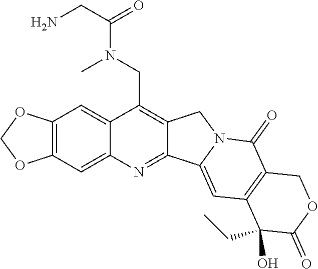 [SMILES: CC[C@@]1(O)C(=O)OCc2c1cc1n(c2=O)Cc2c-1nc1cc3c(cc1c2CN(C)C(=O)CN)OCO3] | 492.16 | 493.17 | 493.00 | 1.4 |

The Camptothecin compounds of Table H are exemplary compounds of formula W-CPT that are incorporated into Camptothecin Conjugates of Camptothecin in which Q-D is of formula —Z-A-S*—W-D or —Z-A-B—(S*)—W-D or are incorporated into Drug Linker compounds of formula Z′-A-S*—W-D or Z′-A-B(S*)—W-D through covalent attachment to the oxygen atom or nitrogen atom of the primary hydroxy or amine functional group, respectively.

### Synthetic Example 7

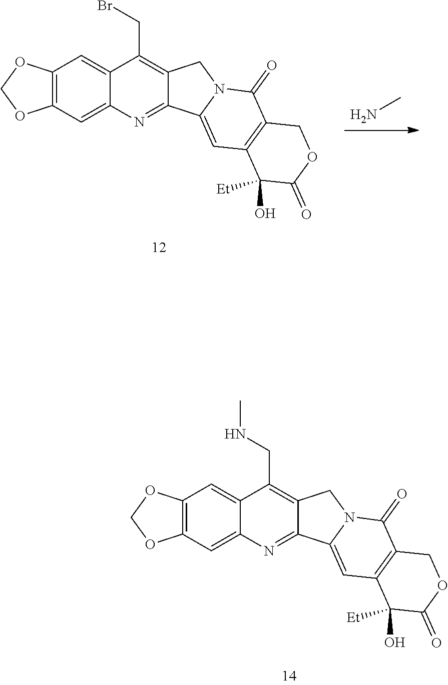

Compound 12 (10.0 mg, 20.6 μmol) from Example 4 was dissolved in anhydrous DMF (0.25 mL). Methylamine (2M in THF, 0.031 mL, 62 μmol) was added. The reaction was stirred for 30 minutes, then quenched with AcOH (20 μL). The reaction was purified by prep-HPLC. Fractions containing the desired product (14) were lyophilized to afford a yellow solid (3.27 mg, 7.51 μmol, 36%). LC-MS (Method D): t_{R}=1.57 min; MS (m/z) [M+H]⁺ calc. for C₉H₉BrNO₃ 257.98, found 257.71. t_{R}=0.93 min (Method A); MS (m/z) [M+H]⁺ calc. for C₂₃H₂₂N₃O₆ 436.15, found 435.78.

TABLE I
Other camptothecin compounds prepared according Synthetic Example 7

| | | | Calc'd MS | Obsv'd | |
|---|---|---|---|---|---|
| | | Parent Exact | (m/z) | MS | |
| No. | Structure | Mass | [M + H] | (m/z) | t_{R}* |
| 14a | 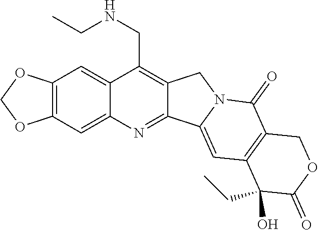 [SMILES: CCNCc1c2c(nc3cc4c(cc13)OCO4)-c1cc3c(c(=O)n1C2)COC(=O)[C@]3(O)CC] | 449.158685 | 450.17 | 450.14 | 1.19 |
| 14b | 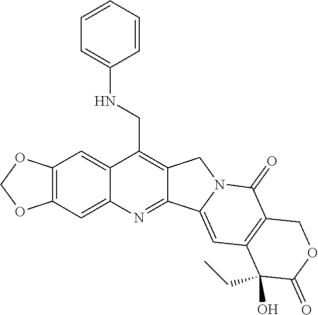 [SMILES: CC[C@@]1(O)C(=O)OCc2c1cc1n(c2=O)Cc2c-1nc1cc3c(cc1c2CNc1ccccc1)OCO3] | 497.158685 | 498.17 | 498.05 | 1.22 |
| 14c | 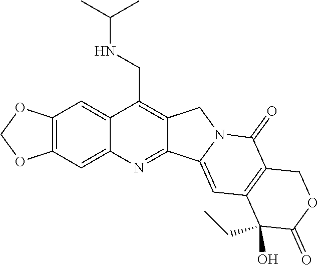 [SMILES: CC[C@@]1(O)C(=O)OCc2c1cc1n(c2=O)Cc2c-1nc1cc3c(cc1c2CNC(C)C)OCO3] | 463.174336 | 464.18 | 464.00 | 0.98 |
| 14d | 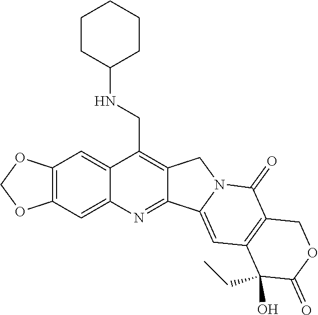 [SMILES: CC[C@@]1(O)C(=O)OCc2c1cc1n(c2=O)Cc2c-1nc1cc3c(cc1c2CNC1CCCCC1)OCO3] | 503.205636 | 504.22 | 504.16 | 1.16 |
| 14e | 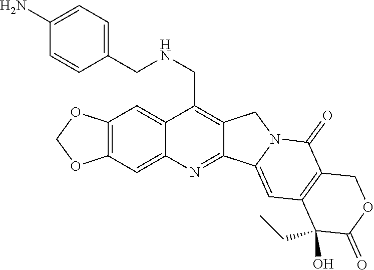 [SMILES: CC[C@@]1(O)C(=O)OCc2c1cc1n(c2=O)Cc2c-1nc1cc3c(cc1c2CNCc1ccc(N)cc1)OCO3] | 526.185235 | 527.20 | 526.08 | 1.11 |
| 14f | 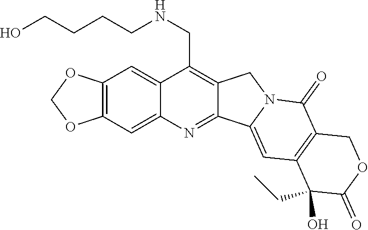 [SMILES: CC[C@@]1(O)C(=O)OCc2c1cc1n(c2=O)Cc2c-1nc1cc3c(cc1c2CNCCCCO)OCO3] | 493.1849 | 494.19 | 493.88 | 1.03 |
| 14g | 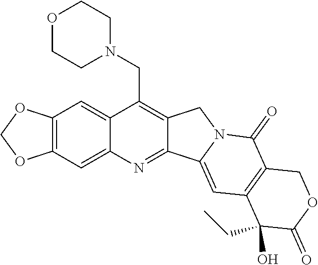 [SMILES: CC[C@@]1(O)C(=O)OCc2c1cc1n(c2=O)Cc2c-1nc1cc3c(cc1c2CN1CCOCC1)OCO3] | 491.16925 | 492.18 | 491.74 | 1.19 |
| 14h | 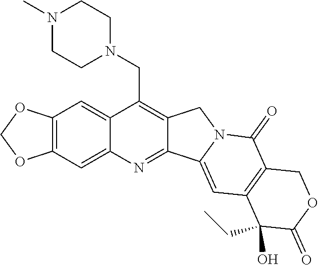 [SMILES: CC[C@@]1(O)C(=O)OCc2c1cc1n(c2=O)Cc2c-1nc1cc3c(cc1c2CN1CCN(C)CC1)OCO3] | 504.200885 | 505.21 | 504.93 | 1.10 |
| 14i | 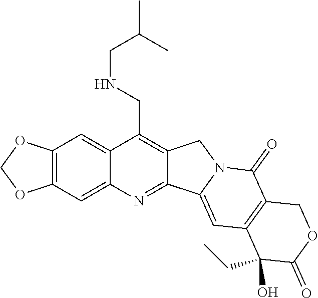 [SMILES: CC[C@@]1(O)C(=O)OCc2c1cc1n(c2=O)Cc2c-1nc1cc3c(cc1c2CNCC(C)C)OCO3] | 477.189986 | 478.20 | 478.26 | 1.30 |
| 14j | 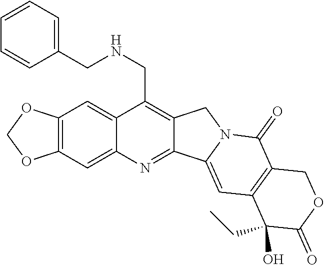 [SMILES: CC[C@@]1(O)C(=O)OCc2c1cc1n(c2=O)Cc2c-1nc1cc3c(cc1c2CNCc1ccccc1)OCO3] | 511.174336 | 512.18 | 512.21 | 1.20 |
| 14k | 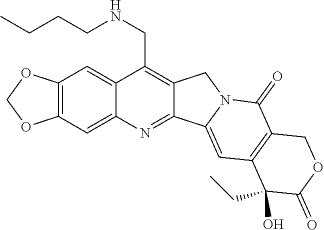 [SMILES: CCCCNCc1c2c(nc3cc4c(cc13)OCO4)-c1cc3c(c(=O)n1C2)COC(=O)[C@]3(O)CC] | 477.189986 | 478.20 | 477.68 | 1.13 |
| 14l | 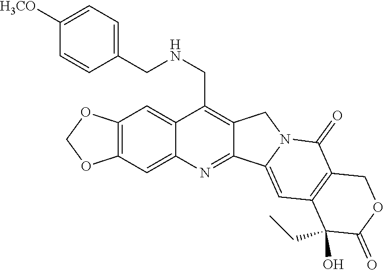 [SMILES: CC[C@@]1(O)C(=O)OCc2c1cc1n(c2=O)Cc2c-1nc1cc3c(cc1c2CNCc1ccc(OC)cc1)OCO3] | 541.1849 | 542.19 | 542.37 | 1.30 |
| 14m | 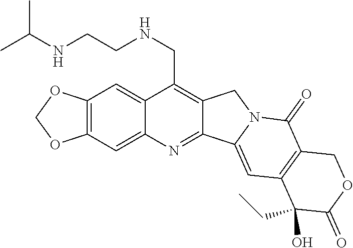 [SMILES: CC[C@@]1(O)C(=O)OCc2c1cc1n(c2=O)Cc2c-1nc1cc3c(cc1c2CNCCNC(C)C)OCO3] | 506.216535 | 507.23 | 507.94 | 0.76 |
| 14n | 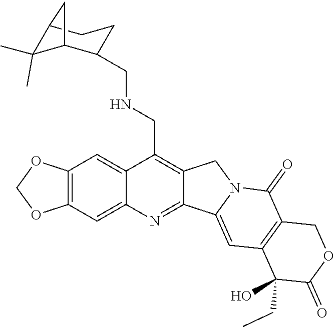 [SMILES: CC[C@@]1(O)C(=O)OCc2c1cc1n(c2=O)Cc2c-1nc1cc3c(cc1c2CNCC1CCC2CC1C2(C)C)OCO3] | 557.252586 | 558.26 | 557.89 | 1.51 |
| 14o | 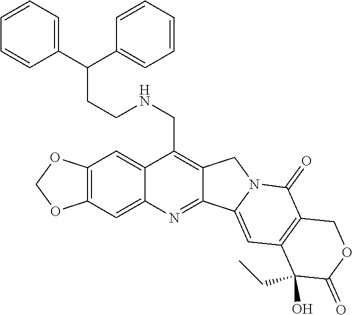 [SMILES: CC[C@@]1(O)C(=O)OCc2c1cc1n(c2=O)Cc2c-1nc1cc3c(cc1c2CNCCC(c1ccccc1)c1ccccc1)OCO3] | 615.236936 | 616.25 | 615.60 | 1.56 |
| 14p | 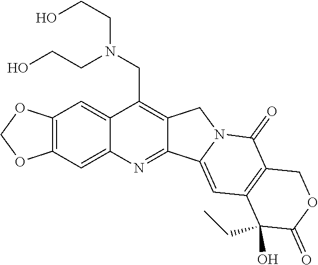 [SMILES: CC[C@@]1(O)C(=O)OCc2c1cc1n(c2=O)Cc2c-1nc1cc3c(cc1c2CN(CCO)CCO)OCO3] | 509.179815 | 510.19 | 509.69 | 1.09 |
| 14q | 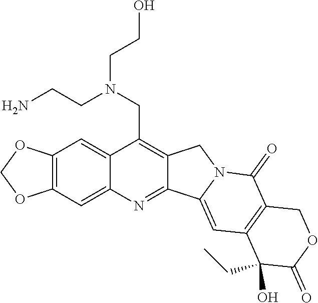 [SMILES: CC[C@@]1(O)C(=O)OCc2c1cc1n(c2=O)Cc2c-1nc1cc3c(cc1c2CN(CCN)CCO)OCO3] | 508.195799 | 509.21 | 508.91 | 1.11 |
| 14r | 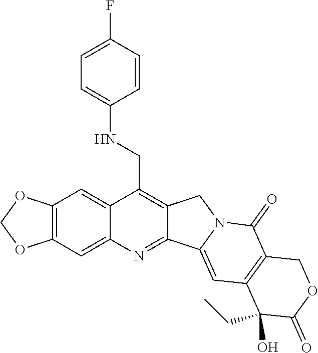 [SMILES: CC[C@@]1(O)C(=O)OCc2c1cc1n(c2=O)Cc2c-1nc1cc3c(cc1c2CNc1ccc(F)cc1)OCO3] | 515.149264 | 516.16 | 515.09 | 1.33 |
| 14s | 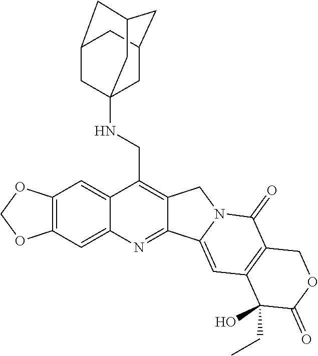 [SMILES: CC[C@@]1(O)C(=O)OCc2c1cc1n(c2=O)Cc2c-1nc1cc3c(cc1c2CNC12CC4CC(CC(C4)C1)C2)OCO3] | 555.236936 | 556.25 | 555.85 | 1.49 |
| 14t | 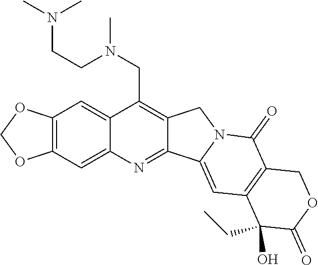 [SMILES: CC[C@@]1(O)C(=O)OCc2c1cc1n(c2=O)Cc2c-1nc1cc3c(cc1c2CN(C)CCN(C)C)OCO3] | 506.216535 | 507.23 | 506.58 | 1.17 |
| 14u | 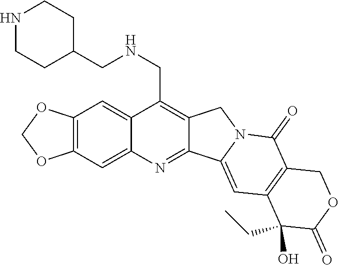 [SMILES: CC[C@@]1(O)C(=O)OCc2c1cc1n(c2=O)Cc2c-1nc1cc3c(cc1c2CNCC1CCNCC1)OCO3] | 518.216535 | 519.23 | 519.09 | 1.00 |
| 14v | 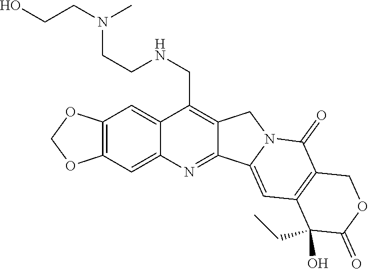 [SMILES: CC[C@@]1(O)C(=O)OCc2c1cc1n(c2=O)Cc2c-1nc1cc3c(cc1c2CNCCN(C)CCO)OCO3] | 522.211449 | 523.22 | 522.68 | 1.04 |
| 14w | 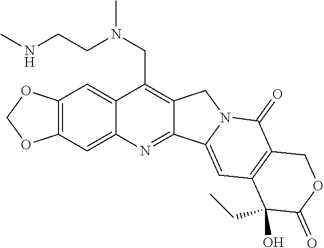 [SMILES: CC[C@@]1(O)C(=O)OCc2c1cc1n(c2=O)Cc2c-1nc1cc3c(cc1c2CN(C)CCNC)OCO3] | 492.200885 | 493.21 | 492.71 | 1.07 |
| 14x | 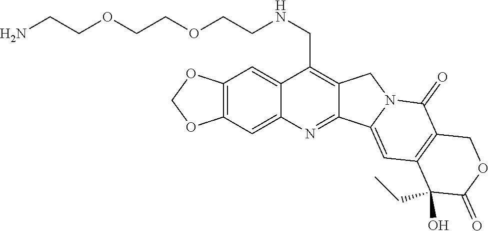 [SMILES: CC[C@@]1(O)C(=O)OCc2c1cc1n(c2=O)Cc2c-1nc1cc3c(cc1c2CNCCOCCOCCN)OCO3] | 552.222014 | 553.23 | 553.14 | 1.08 |
| 14y | 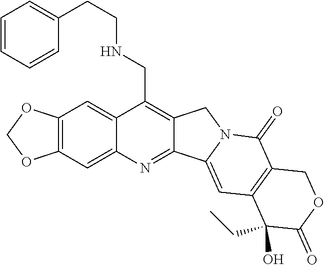 [SMILES: CC[C@@]1(O)C(=O)OCc2c1cc1n(c2=O)Cc2c-1nc1cc3c(cc1c2CNCCc1ccccc1)OCO3] | 525.189986 | 526.20 | 525.59 | 1.31 |
| 14z | 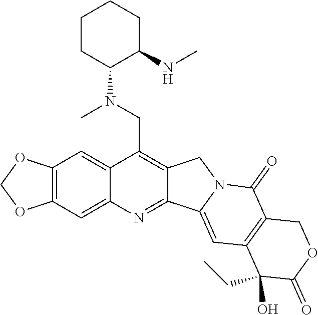 [SMILES: CC[C@@]1(O)C(=O)OCc2c1cc1n(c2=O)Cc2c-1nc1cc3c(cc1c2CN(C)[C@@H]1CCCC[C@H]1NC)OCO3] | 546.247835 | 547.26 | 546.64 | 1.26 |

*LC-MS Method A

### Synthetic Example 8

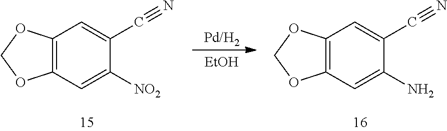 [SMILES: "EtOH" ; N#Cc1cc2c(cc1[N+](=O)[O-])OCO2 ; "Pd/H2" ; N#Cc1cc2c(cc1N)OCO2]

As described in Heterocycles, (2007) 71: 39-48) 6-nitro-1,3-benzodioxole-5-carbonitrile (compound 15, 2.00 g, 10.4 mmol) was prepared and then dissolved in EtOH (50 mL). Reaction was placed under nitrogen atmosphere. Pd/C (2.22 g, 10% w/w, 2.08 mmol) added to the reaction, which was placed under hydrogen atmosphere. The reaction was stirred for 2 hours. The reaction was filtered through a bed of Celite, then rinsed with MeOH. The eluent was concentrated in vacuo and purified by flash chromatography 0-10% DCM in MeOH. Fractions containing the desired product were concentrated to afford compound 16 as a red solid (1.46 g, 9.00 mmol, 87%). LC-MS (Method D): t_{R}=1.14 min; MS (m/z) [M+H]⁺ calc. for C₈H₇N₂O₂ 163.05, found 162.37.

### Synthetic Example 9

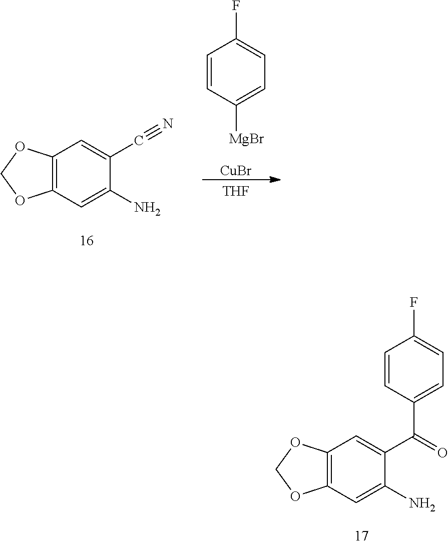

6-amino-1,3-benzodioxole-5-carbonitrile (compound 16, 50 mg, 0.31 mmol) was placed under nitrogen atmosphere and dissolved in anhydrous THE (1 mL). CuBr (1.5 mg, 0.010 mmol) was added followed by 4-fluorophenylmagnesium bromide 1M in THE (1.23 mL). The reaction was heated to 60° C. for 30 minutes, and then cooled to room temperature. A solution of 15% H₂SO₄ was added to the reaction slowly, then stirred for 30 minutes. The reaction was poured into sat. NaHCO₃ (50 mL), then extracted with EtOAc (3×50 mL). The organic was dried with MgSO₄, filtered and concentrated in vacuo. The crude product was purified by column chromatography 10 G Biotage Ultra 0-10% EtOAc in Hex. Fractions containing the desired product were concentrated in vacuo to afford compound 17 as a red solid (46.2 mg, 0.178 mmol, 58%). LC-MS (Method D): t_{R}=1.81 min; MS (m/z) [M+H]⁺ calc. for C₁₄H₁₁FNO₃ 260.07, found 259.46.

### Synthetic Example 10

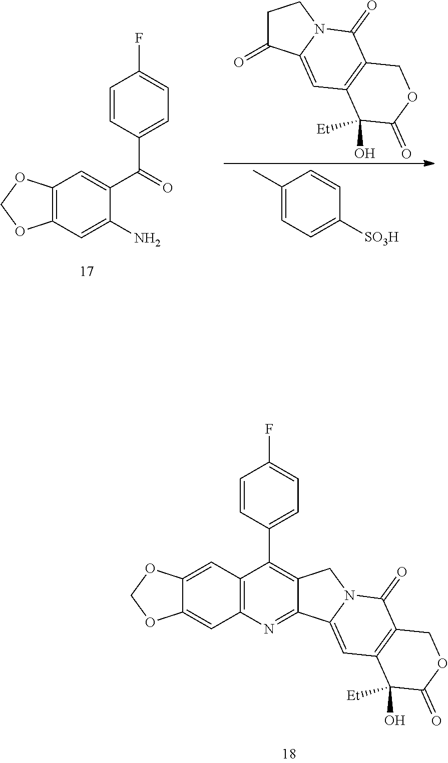

Compound 17 (46.2 mg, 0.178 mmol), p-TSA (30.7 mg, 0.178 mmol), and 4-Ethyl-4-hydroxy-7,8-dihydro-1H-pyrano[3,4-f]indolizine-3,6,10(4H)-trione (46.9 mg, 0.178 mmol, obtained from Avra Laboratories Pvt. Ltd.) were charged in a scintillation vial. DCM (1 mL) was added to homogenate the solids. The solvent was concentrated under nitrogen. The neat solids were the heated to 120° C. under high vacuum (1 mbar) for 60 minutes. The reaction was reconstituted in DCM (50 mL), washed with H₂O, the organic phase wash dried with MgSO₄, filtered and concentrated in vacuo. The crude product was purified by column chromatography 10 G Biotage Ultra 0-10% MeOH in DCM. Fractions containing the desired product (18) were concentrated in vacuo to afford a red solid (32.9 mg, 0.0676 mmol, 38%). LC-MS (Method D): t_{R}=1.81 min; MS (m/z) [M+H]⁺ calc. for C₂₇H₂₀FN₂O₆ 487.13, found 487.19.

TABLE J
Other camptothecin compounds prepared according to the procedure of
Examples 9 and 10.

| | | | Calc'd | Observed | |
|---|---|---|---|---|---|
| | | Parent Exact | MS (m/z) | MS | |
| No. | Structure | Mass | [M + H]⁺ | (m/z) | t_{R}* |
| 18a | 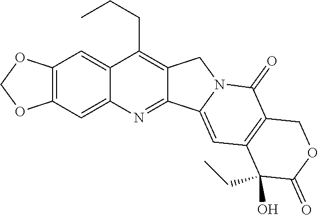 [SMILES: CCCc1c2c(nc3cc4c(cc13)OCO4)-c1cc3c(c(=O)n1C2)COC(=O)[C@]3(O)CC] | 434.147786 | 435.16 | 434.81 | 1.62 |
| 18b | 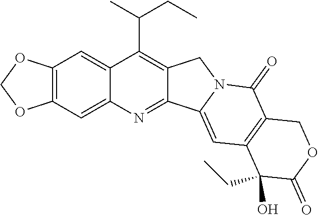 [SMILES: CCC(C)c1c2c(nc3cc4c(cc13)OCO4)-c1cc3c(c(=O)n1C2)COC(=O)[C@]3(O)CC] | 448.163437 | 449.17 | 448.78 | 1.71 |
| 18c | 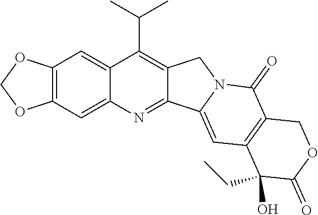 [SMILES: CC[C@@]1(O)C(=O)OCc2c1cc1n(c2=O)Cc2c-1nc1cc3c(cc1c2C(C)C)OCO3] | 434.147786 | 435.16 | 434.81 | 1.59 |
| 18d | 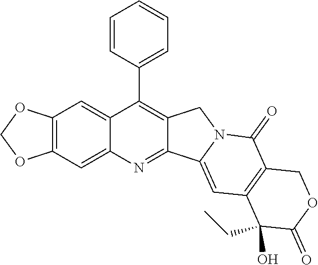 [SMILES: CC[C@@]1(O)C(=O)OCc2c1cc1n(c2=O)Cc2c-1nc1cc3c(cc1c2-c1ccccc1)OCO3] | 468.132136 | 469.14 | 469.15 | 1.77 |
| 18e | 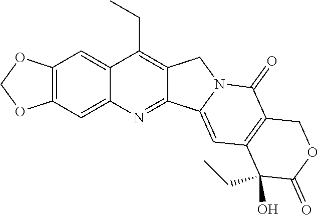 [SMILES: CCc1c2c(nc3cc4c(cc13)OCO4)-c1cc3c(c(=O)n1C2)COC(=O)[C@]3(O)CC] | 420.132136 | 421.14 | 420.85 | 1.48 |
| 18f | 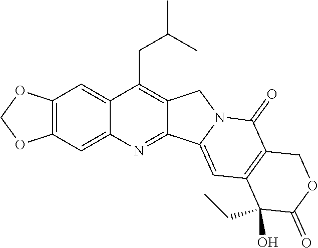 [SMILES: CC[C@@]1(O)C(=O)OCc2c1cc1n(c2=O)Cc2c-1nc1cc3c(cc1c2CC(C)C)OCO3] | 448.163437 | 449.17 | 448.78 | 1.76 |
| 18g | 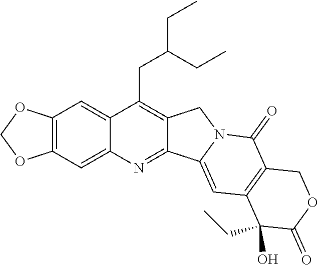 [SMILES: CCC(CC)Cc1c2c(nc3cc4c(cc13)OCO4)-c1cc3c(c(=O)n1C2)COC(=O)[C@]3(O)CC] | 476.194737 | 477.20 | 476.81 | 2.00 |
| 18h | 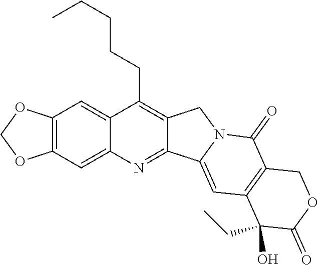 [SMILES: CCCCCc1c2c(nc3cc4c(cc13)OCO4)-c1cc3c(c(=O)n1C2)COC(=O)[C@]3(O)CC] | 462.179087 | 463.19 | 462.94 | 1.93 |
| 18i | 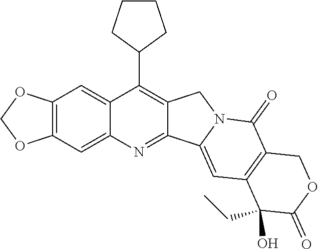 [SMILES: CC[C@@]1(O)C(=O)OCc2c1cc1n(c2=O)Cc2c-1nc1cc3c(cc1c2C1CCCC1)OCO3] | 460.163437 | 461.17 | 460.80 | 1.79 |
| 18j | 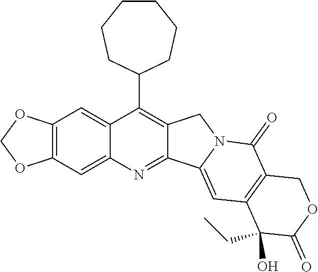 [SMILES: CC[C@@]1(O)C(=O)OCc2c1cc1n(c2=O)Cc2c-1nc1cc3c(cc1c2C1CCCCCC1)OCO3] | 488.194737 | 489.20 | 489.12 | 2.03 |
| 18k | 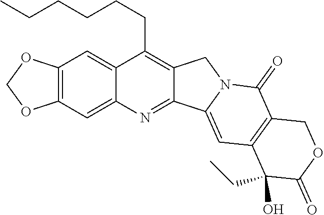 [SMILES: CCCCCCc1c2c(nc3cc4c(cc13)OCO4)-c1cc3c(c(=O)n1C2)COC(=O)[C@]3(O)CC] | 476.194737 | 477.20 | 478.07 | 2.06 |
| 18l | 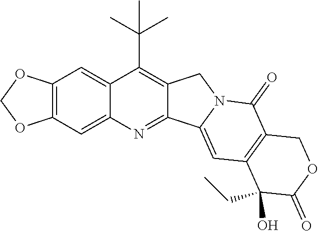 [SMILES: CC[C@@]1(O)C(=O)OCc2c1cc1n(c2=O)Cc2c-1nc1cc3c(cc1c2C(C)(C)C)OCO3] | 448.163437 | 449.17 | 448.87 | 1.69 |
| 18m | 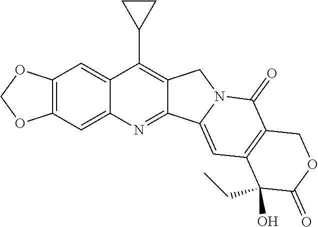 [SMILES: CC[C@@]1(O)C(=O)OCc2c1cc1n(c2=O)Cc2c-1nc1cc3c(cc1c2C1CC1)OCO3] | 432.132136 | 433.14 | 433.16 | 1.56 |
| 18n | 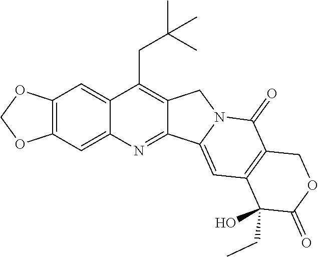 [SMILES: CC[C@@]1(O)C(=O)OCc2c1cc1n(c2=O)Cc2c-1nc1cc3c(cc1c2CC(C)(C)C)OCO3] | 462.179087 | 463.19 | 463.04 | 1.83 |
| 18o | 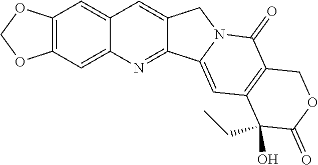 [SMILES: CC[C@@]1(O)C(=O)OCc2c1cc1n(c2=O)Cc2cc3cc4c(cc3nc2-1)OCO4] | 392.100836 | 393.11 | 393.01 | 1.31 |
| 18p | 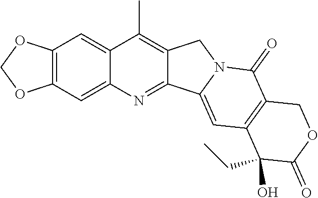 [SMILES: CC[C@@]1(O)C(=O)OCc2c1cc1n(c2=O)Cc2c-1nc1cc3c(cc1c2C)OCO3] | 406.12 | 407.13 | 407.07 | 1.42 |
| 18q | 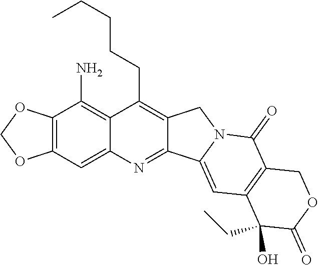 [SMILES: CCCCCc1c2c(nc3cc4c(c(N)c13)OCO4)-c1cc3c(c(=O)n1C2)COC(=O)[C@]3(O)CC] | 477.18999 | 478.20 | 478.17 | 1.26 |
| 18r | 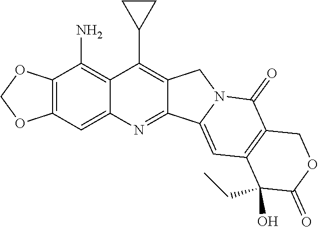 [SMILES: CC[C@@]1(O)C(=O)OCc2c1cc1n(c2=O)Cc2c-1nc1cc3c(c(N)c1c2C1CC1)OCO3] | 447.14304 | 448.15 | 448.00 | 1.33 |

*LC-MS Method A except 18q (Method E) and 18r (Method D)

### Synthetic Example 11

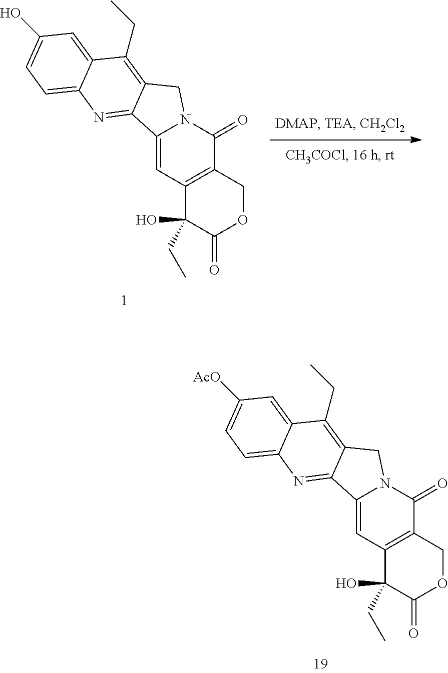 [SMILES: CCc1c2c(nc3ccc(O)cc13)-c1cc3c(c(=O)n1C2)COC(=O)[C@]3(O)CC ; CCc1c2c(nc3ccc(OC(C)=O)cc13)-c1cc3c(c(=O)n1C2)COC(=O)[C@]3(O)CC]

SN-38 (compound 1, 76.0 mg, 0.19 mmol), obtained from MedChemExpress, was dissolved in dichloromethane, followed by addition of triethylamine (128 μL, 0.92 mmol) and DMAP (2.60 mg, 0.02 mmol). Mixture was cooled to 0° C. in an ice bath, followed by dropwise addition of acetyl chloride (15.9 μL, 0.22 mmol). The reaction mixture was stirred at room temperature for 16 h. The reaction was diluted with dichloromethane, washed with saturated NH₄Cl, water, and brine. The organic phase was then dried over MgSO₄, filtered, concentrated and purified over silica via Biotage flash column chromatography (CH₂Cl₂/MeOH 0-15%) to yield acetylated SN-38 (19). MS (m/z) calculated 435.15 (M+H)⁺, found 435.07.

TABLE K
Camptothecin compounds prepared as described herein:

| | | | Calc'd | | |
|---|---|---|---|---|---|
| Ex. | | Parent Exact | MS (m/z) | Observed | |
| No. | Structure | Mass | [M + H]⁺ | MS (m/z) | t_{R} |
| 20a | 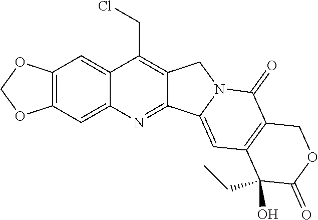 [SMILES: CC[C@@]1(O)C(=O)OCc2c1cc1n(c2=O)Cc2c-1nc1cc3c(cc1c2CCl)OCO3] | 440.077514 | 441.09 | 441.02 | 1.49 |
| 20b | 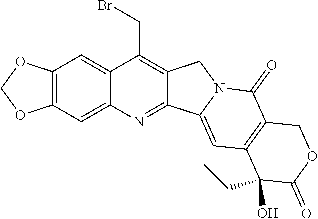 [SMILES: CC[C@@]1(O)C(=O)OCc2c1cc1n(c2=O)Cc2c-1nc1cc3c(cc1c2CBr)OCO3] | 484.026999 | 485.04 | 484.95 | 1.53 |
| 20c | 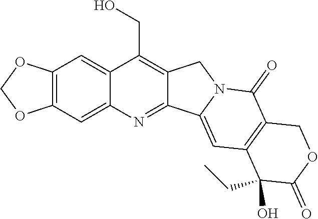 [SMILES: CC[C@@]1(O)C(=O)OCc2c1cc1n(c2=O)Cc2c-1nc1cc3c(cc1c2CO)OCO3] | 422.11 | 423.12 | 423.04 | 1.29 |

### Synthetic Example 12

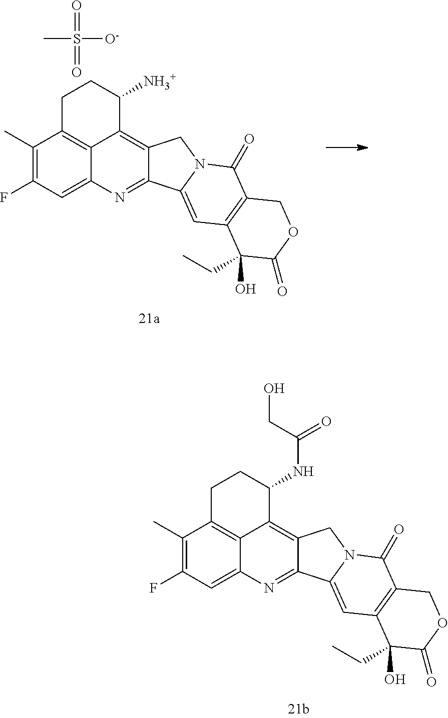

Exatecan mesylate (compound 21a) 20.0 mg, 0.0376 mmol, obtained from MedChemExpress Cat. No.: HY-13631A) was suspended in anhydrous DCM (1 mL). DIPEA (20.0 μL, 0.0146 mmol) was added followed by acetoxyacetyl chloride (5.0 μL, 0.046 mmol). The reaction was stirred for 30 minutes, then quenched with MeOH, and concentrated in vacuo. The reaction mixture was re-dissolved in MeOH (1 mL). LiOH (20 mg) added. Complete deprotection of acetate observed. Quenched AcOH. Purified by Prep-HPLC 10 mm 10-95% MeCN in H₂O 0.05% TFA. Fractions containing the desired product were concentrated in vacuo to afford compound 21b as a yellow solid (15.3 mg, 0.0310 mmol, 82%). LC-MS (Method A): t_{R}=1.46 min; MS (m/z) [M+H]⁺ calc. for C₂₆H₂₅N₃O₆ 494.17, found 494.05.

### Synthetic Example 13

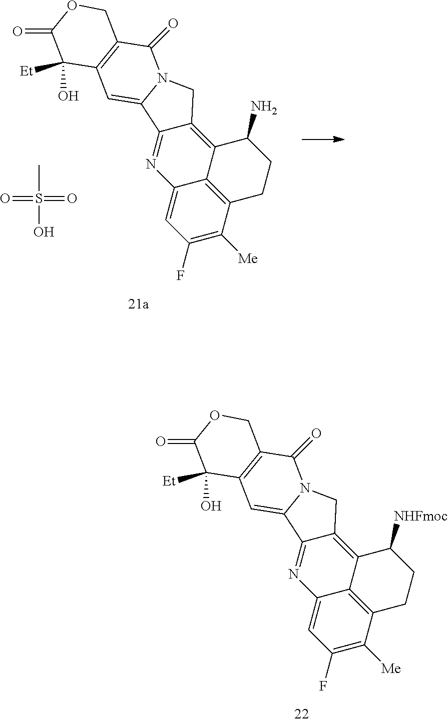

Exatecan mesylate (compound 21a, 20.0 mg, 0.0376 mol) was dissolved in MeCN (1 mL) and NaHCO₃ 0.75M in H₂O. Fmoc-OSu (19.0 mg, 0.0564 mmol) was added and the reaction was stirred for 2 hours 30 minutes. The reaction was diluted with H₂O (50 mL), the pH was adjusted to neutral and extracted with DCM (3×50 mL). The organic phase was dried with MgSO₄, filter and concentrated in vacuo. The crude product was purified by prep-TLC 0-5% MeOH in DCM. The band containing the desired product was scraped, filtered washing with 10% MeOH in DCM, and the eluent was concentrated in vacuo to afford compound 22 as an orange solid (19.1 mg, 0.0290 mmol, 77%). LC-MS (Method A): t_{R}=2.23 min; MS (m/z) [M+H]⁺ calc. for C₃₉H₃₃FN₃O₆ 658.24, found 658.09.

### Synthetic Example 14

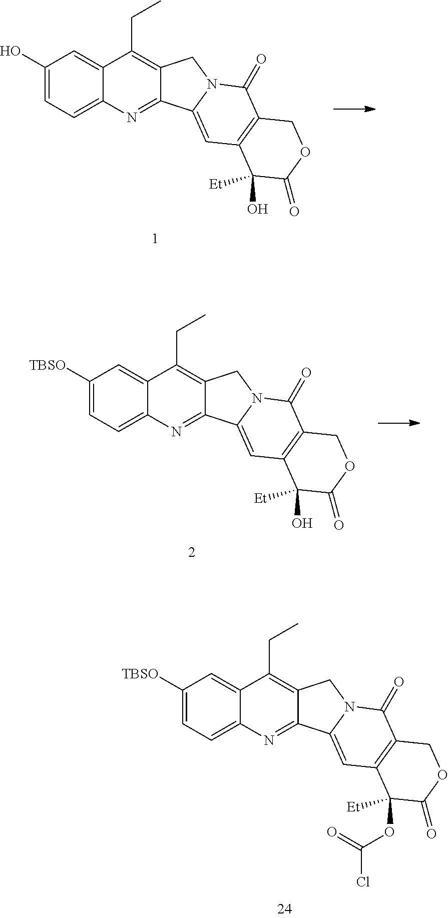 [SMILES: CCc1c2c(nc3ccc(O)cc13)-c1cc3c(c(=O)n1C2)COC(=O)[C@]3(O)CC ; CCc1c2c(nc3ccc(O[Si](C)(C)C(C)(C)C)cc13)-c1cc3c(c(=O)n1C2)COC(=O)[C@]3(O)CC ; CCc1c2c(nc3ccc(O[Si](C)(C)C(C)(C)C)cc13)-c1cc3c(c(=O)n1C2)COC(=O)[C@@]3(CC)OC(=O)Cl]

Compound 2 (132 mg, 0.260 mmol), prepared according to Example 1, was dissolved in 2 mL anhydrous DCM. DMAP (111 mg, 0.911 mmol) was added. Triphosgene (34.8 mg, 0.117 mmol) dissolved 50 mg/mL in anhydrous DCM, and the solution was added dropwise to stirred reaction solution over 5 minutes. A 2 μL aliquot of the reaction solution was quenched into 98 μL MeOH diluent. Nearly complete conversion was observed to the Me-carbonate by UPLC-MS after 15 minutes. Reaction mixture containing compound 24 was used immediately in coupling reactions described herein.

### Synthetic Example C. Camptothecin Drug Linker Compound Preparations

### Synthetic Example 15

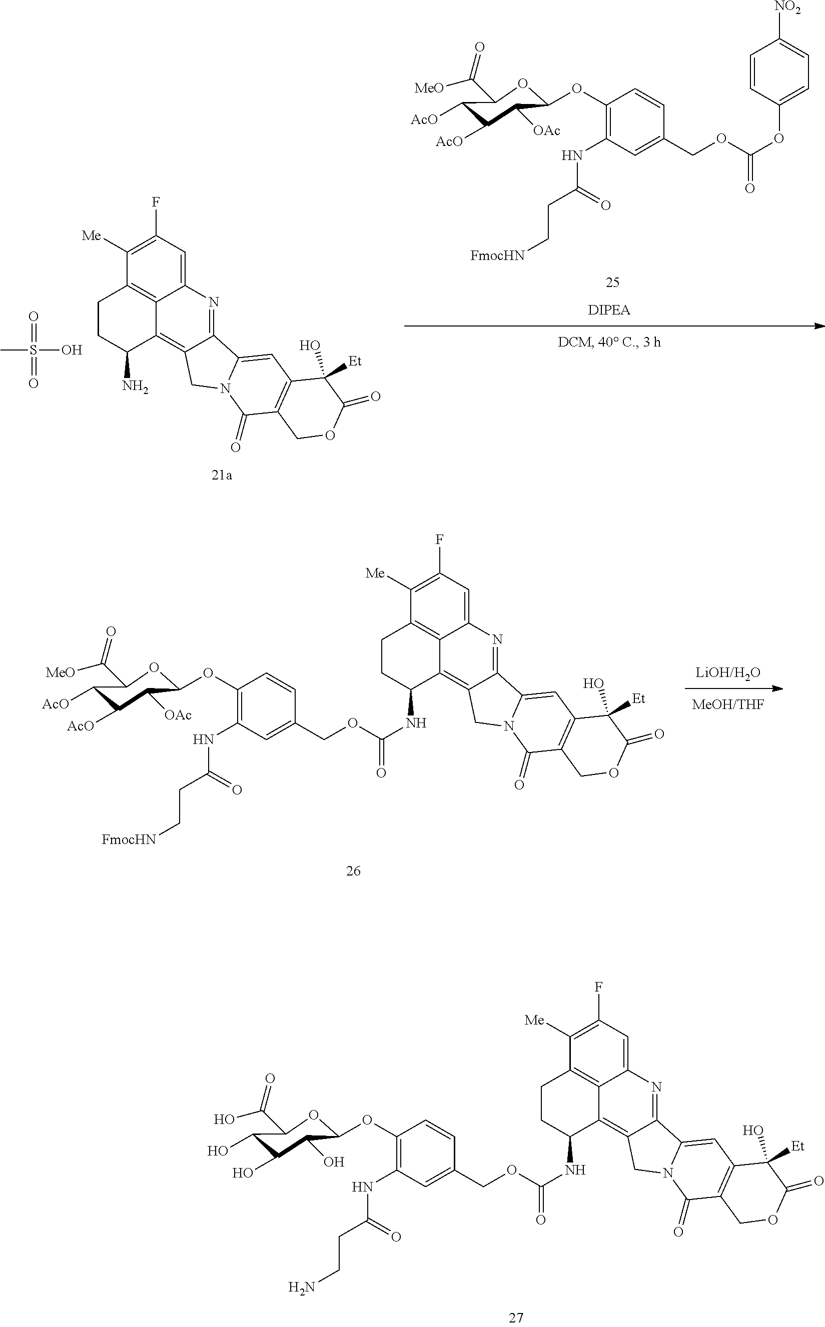

Exatecan mesylate (compound 21a, 5.00 mg, 9.41 μmol) was dissolved in anhydrous DCM. DIPEA (5.0 μL, 28 μmol) was added followed by compound 25 (17.2 mg, 18.8 μmol) previously described by Bioconjugate Chem. (2006) 17: 831-840. The reaction was stirred at 40° C. for 3 hours. The reaction was quenched with MeOH and concentrated in vacuo. The crude reaction mixture was used in the next step. LC-MS (Method A): t_{R}=2.32 min; MS (m/z) [M+H]⁺ calc. for C₆₃H₆₁FN₅O₁₉ 1210.39, found 1210.08.

Crude compound 26 (9.41 μmol) from the previous step was dissolved in THF (1 mL), and 1M LiOH in MeOH (1 mL). The reaction was stirred for 5 minutes, then H₂O was added and stirred for an additional 5 minutes. The reaction was quenched with AcOH (100 μL), concentrated in vacuo and purified by prep-HPLC 21 mm 5-60-95%0 MeCN in H₂O 0.05% TFA. Fractions containing the desired product were lyophilized to afford compound 27 as a yellow powder (1.1 mg, 1.3 μmol). LC-MS (Method A): t_{R}=1.29 min; MS (m/z) [M+H]⁺ calc. for C₄₁H₄₃FN₅O₁₄ 848.28, found 848.03.

### Synthetic Example 16

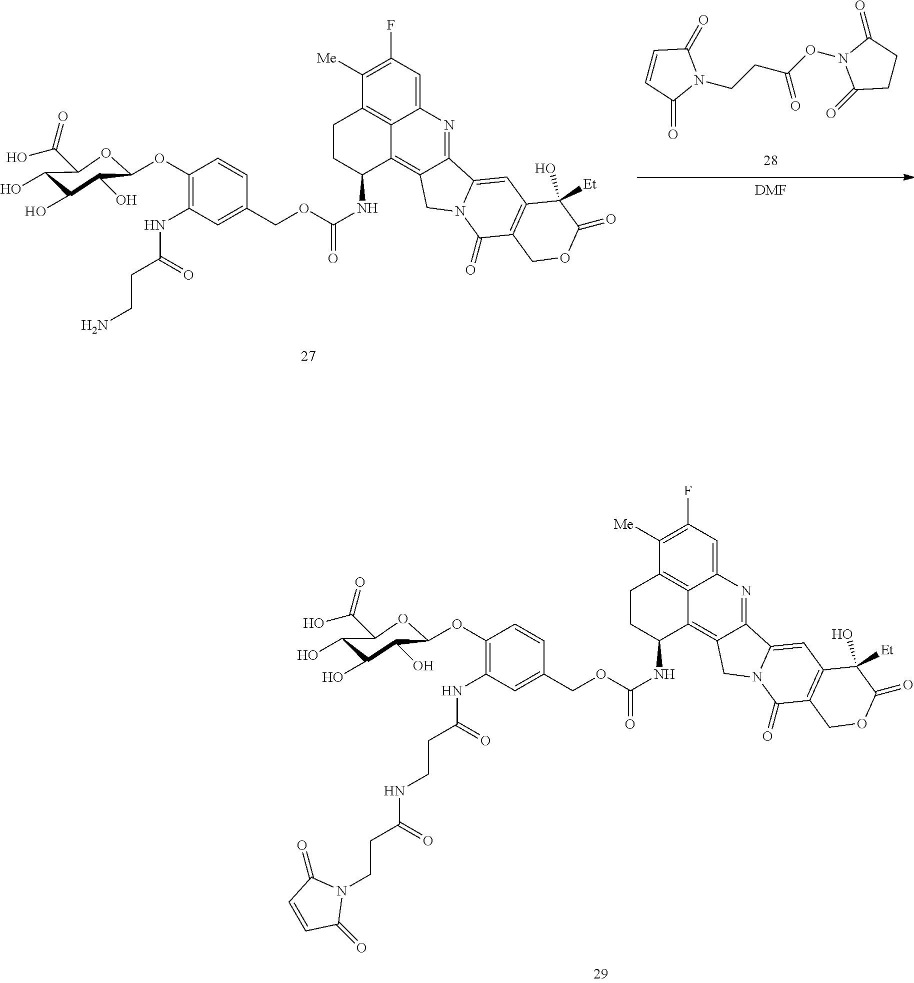

Compound 27 (1.1 mg, 1.3 μmol) was dissolved in anhydrous DMF (0.5 mL). DIPEA (1 μL) was added followed by N-Succinimidyl 3-Maleimidopropionate (28, 0.63 mg, 2.4 μmol) purchased from TCI (CAS: 55750-62-4). The reaction was stirred for 5 minutes. Complete conversion was observed by UPLC-MS. The reaction was quenched with AcOH (10 μL), then purified by prep-HPLC 10 mm 5-60-95% MeCN in H₂O 0.05% TFA. Fractions containing the desired product were lyophilized to afford compound 29 as a yellow powder (1.21 mg, 1.21 μmol, 93%). LC-MS (Method A): t_{R}=1.52 min; MS (m/z) [M+H]⁺ calc. for C₄₈H₄₈FN₆O₁₇ 999.31, found 999.07.

### Synthetic Example 17

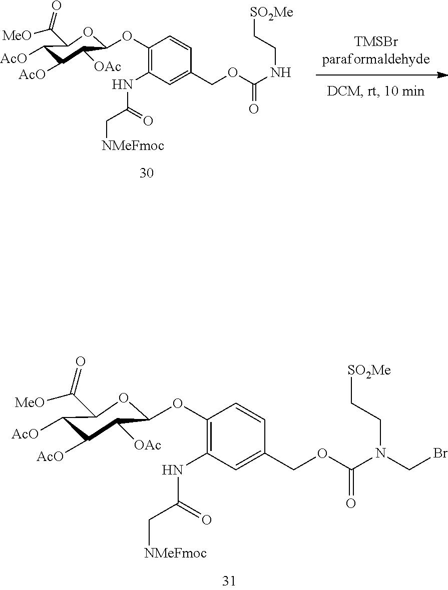

Compound 30 (210 mg, 0.234 mmol), prepared as described by Examples 21 and 22, was dissolved in bench DCM (3 mL). Paraformaldehyde (300-600 mg, xs) was added. Stirring vigorously, TMSBr (0.1 mL) was added. Reaction stirred for 10 minutes at which time complete conversion was observed by UPLC-MS. Reaction mixture was filtered through syringe filter, rinsed DCM (2×3 mL), toluene (3 mL) was added to azeotrope the final mixture. Concentrated in vacuo to afford a white solid. Used in next step without further purification. MeOH diluent was used to observe MeOH quenched adduct by UPLC-MS. LC-MS (Method A): t_{R}=2.19 min; MS (m/z) [M+Na]⁺ calc. for C₄₄H₅₁N₃NaO₁₈S 964.28, found 965.17.

### Synthetic Example 18

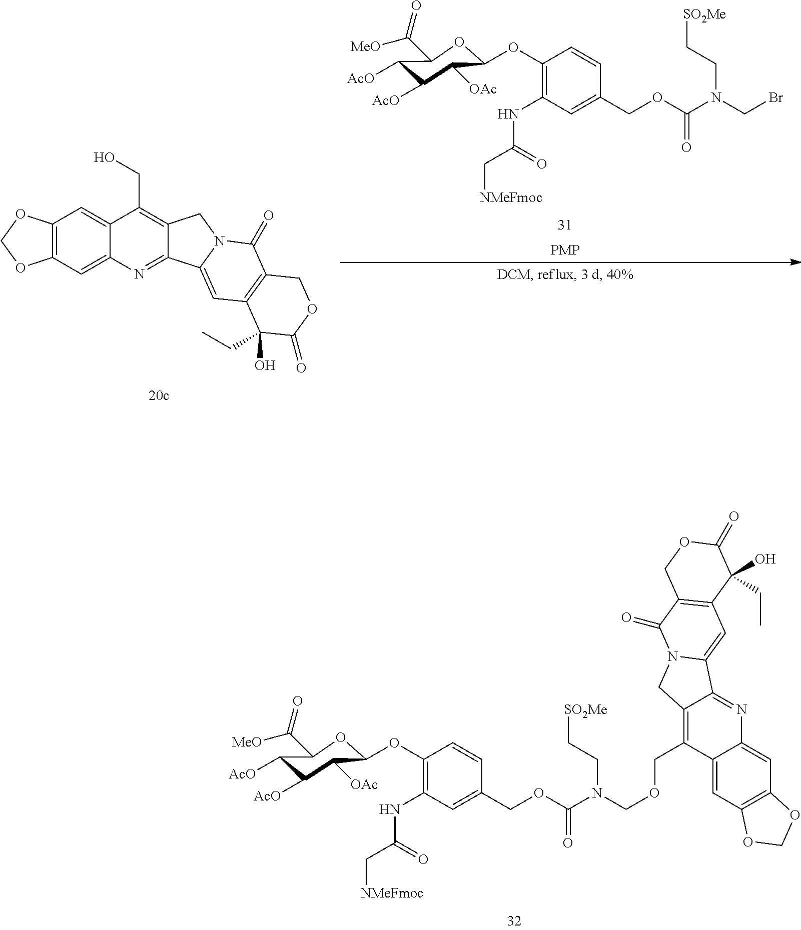

Compound 20c (20 mg, 0.047 mmol), referred to as 7-BAD-MDCPT, was azeotroped 3 times with toluene and dried under high vacuum prior to use. Crude compound 31 (231 mg, 0.234 mmol) from Example 16 was dissolved in anhydrous DCM, and 1,2,2,6,6-pentamethylpiperidine (PMP, 51.4 μL, 0.284 mmol) was added. Minimal hydrolysis of compound 31 in solution was observed by UPLC-MS after base addition. The solution of compound 31 was added directly to the drug reaction vessel, then heated to reflux. Compound 20c is only slightly soluble in DCM. Reaction was monitored by UPLC-MS for completion, which required heating at reflux for 3 days. Afterwards, the reaction was quenched with MeOH, concentrated in vacuo, and purified by FCC Biotage 10 G Ultra 0-10% MeOH in DCM. Fractions containing the desired product (compound 32) were concentrated to afford a yellow solid (50 mg, ˜50% w/w, 0.019 mmol, 40%) as an approximately 50% w/w mixture with dimerized hydrolyzed linker. t_{R}=1.46 min (General Method UPLC); MS (m/z) [M+H]⁺ calc. for C₆₅H₆₆N₅O₂₄S 1332.38, found 1332.54.

### Synthetic Example 19

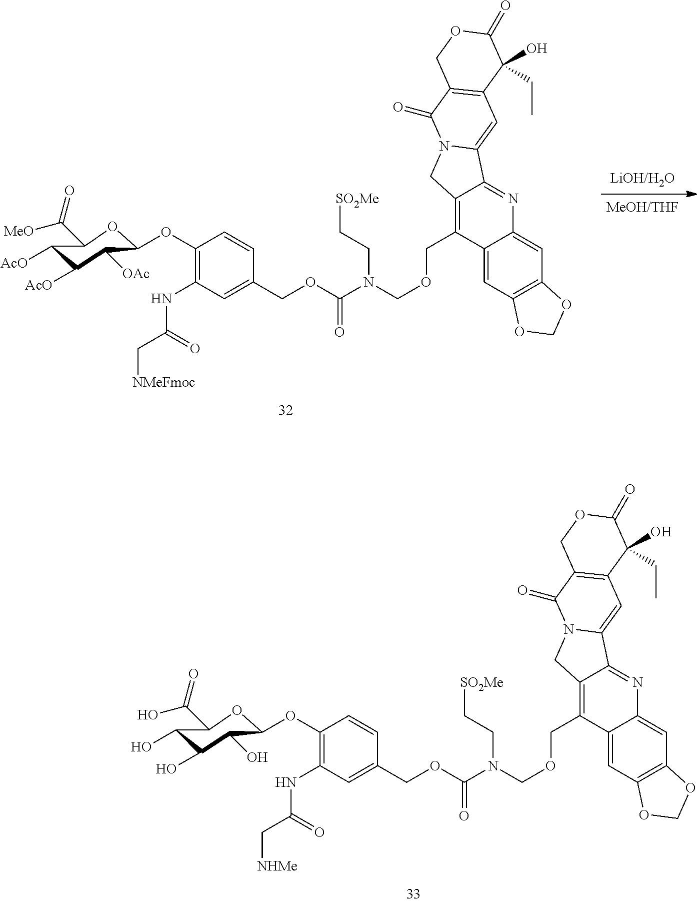

Compound 32 (50 mg, 50% w/w, 0.019 mmol) was dissolved in MeOH:THF 1:1 (1 mL). LiOH (20 mg, 0.84 mmol) was added and stirred for 60 minutes. Water (0.5 mL) was added. Complete conversion was observed by UPLC-MS. Reaction was quenched with AcOH, concentrated in vacuo, and purified by Prep-HPLC. Fractions containing the desired product were lyophilized to afford the desired product, compound 33, as a yellow solid (5 mg, 0.005 mmol, 27%). LC-MS (Method A): t_{R}=0.84 min; MS (m/z) [M+H]⁺ calc. for C₄₃H₄₈N₅O₁₉S 970.27, found 969.92.

### Synthetic Example 20

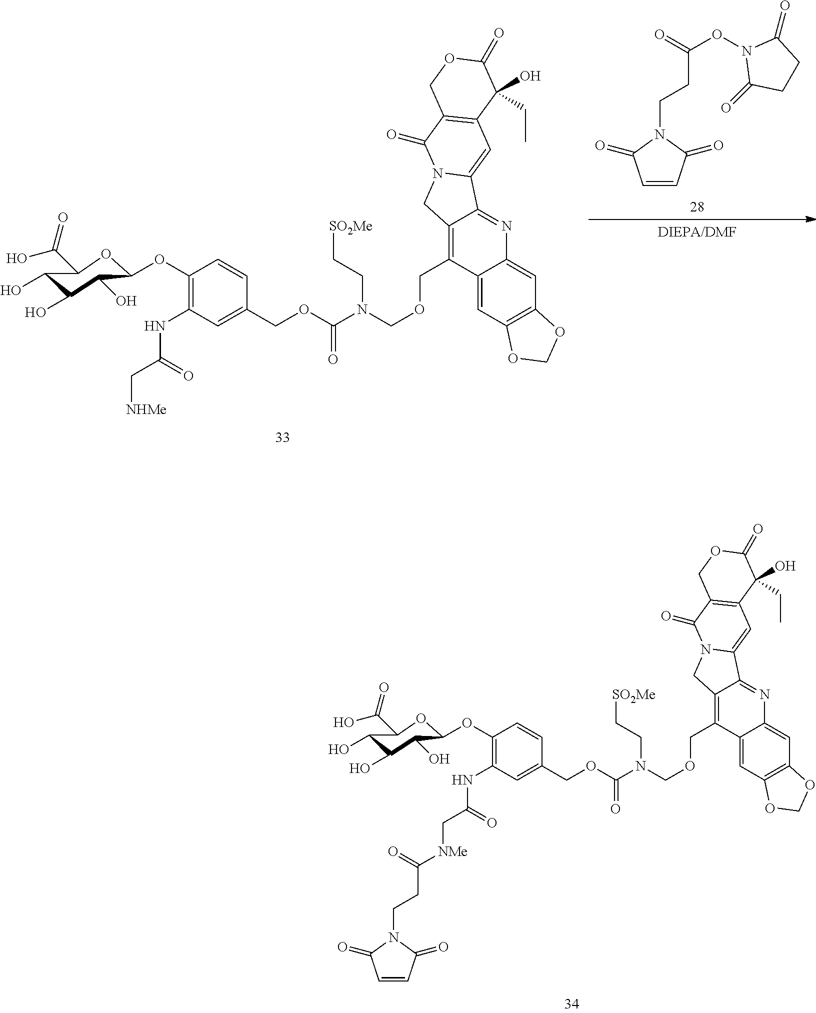

Compound 33 (5 mg, 0.005 mmol) was dissolved in DMF (0.5 mL). DIPEA (10 μL) added followed by 3-(maleimido)-propionic acid N-hydroxysuccinimide ester (28, 4.1 mg, 0.016 mmol) and stirred for 45 minutes at which point complete conversion was observed by UPLC-MS. The reaction was quenched with AcOH (20 μL) and purified by Prep-HPLC 10 mm Max-RP C12 5-60-95% MeCN in H₂O. Fractions containing the desired product compound 34 were lyophilized to afford a yellow powder (2.33 mg, 2.08 μmol, 40.3%). LC-MS (Method A): t_{R}=1.35 min; MS (m/z) [M+H]⁺ calc. for C₅₀H₅₃N₆O₂₂S 1121.29, found 1121.25.

### Synthetic Example 21

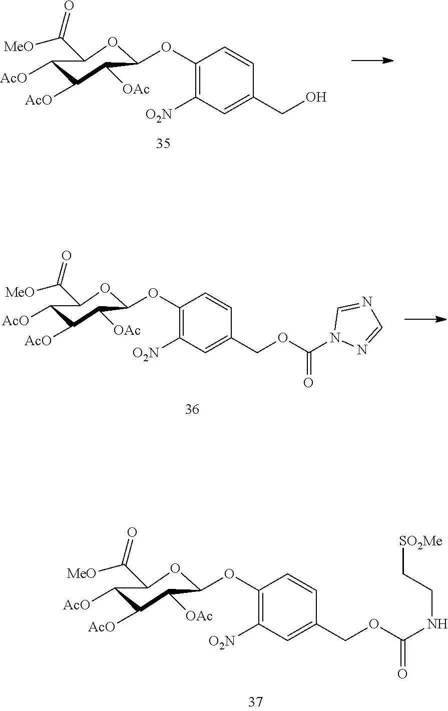

Compound 35 (2.00 g, 4.12 mmol), prepared according to the procedure in Bioconjugate Chem. (2006) 17: 831-840, was dissolved in anhydrous DCM (20 mL). DIPEA (3.59 mL, 20.60 mmol) was added followed by 1,1′-Carbonyl-di-(1,2,4-triazole) (744 mg, 4.53 mmol). The reaction was stirred for 5 minutes, and the reaction mixture containing compound 36 was used in the next step.

To the reaction mixture containing compound 36 (4.12 mmol) was added 2-(methylsulfonyl)-ethanamine (0.61 mL, 6.2 mmol). The reaction as stirred for 5 minutes at which time complete conversion was observed by UPLC-MS. The reaction was concentrated in vacuo and purified by column chromatography KP-Sil 100 G 10-100% EtOAc in Hex. Fractions containing the desired product were concentrated in vacuo to afford compound 37 as a colorless solid (2.40 g, 3.78 mmol, 92%). LC-MS (Method A): t_{R}=1.71 min; MS (m/z) [M+Na]⁺ calc. for C₂₄H₃₀N₂NaO₁₆S 657.12, found 656.93.

### Synthetic Example 22

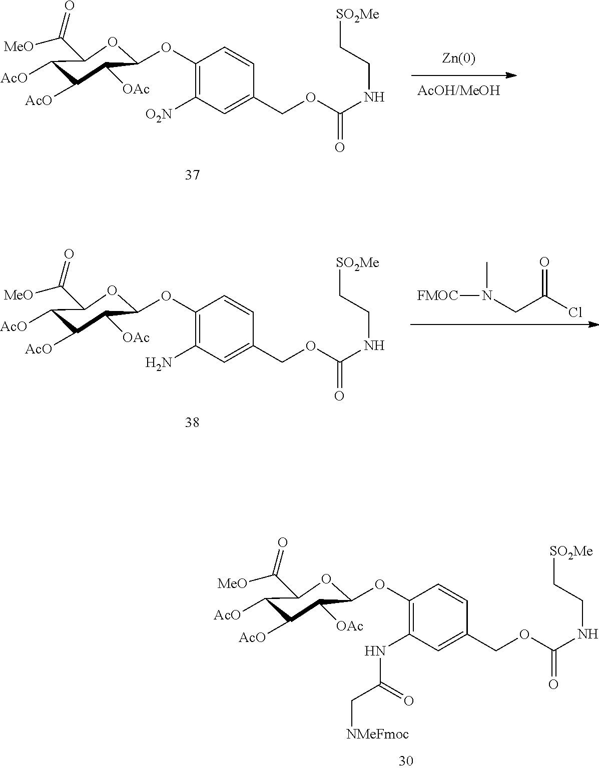

Compound 37 (2.40 g, 3.78 mmol) was dissolved in MeOH (20 mL). AcOH (10 mL) was added to the reaction followed by zinc dust (7.42 g, 113 mmol). The reaction was stirred for 20 minutes at which time complete conversion was observed by UPLC-MS. The reaction was filtered through silica eluting with 20% MeOH in DCM. The eluent was concentrated and used in the next step.

Crude compound 38 (3.78 mmol) was dissolved in anhydrous DCM (10 ml). DIPEA (3.30 mL, 18.9 mmol) was added followed by Fmoc-Sar-Cl (2.50 g, 7.58 mmol). The reaction was stirred for 5 minutes at which point complete conversion was observed by UPLC-MS. The reaction was quenched MeOH, concentrated in vacuo and purified by column chromatography KP-Sil 100 G 10-100% EtOAc in Hex. Fractions containing the desired product were concentrated in vacuo to afford compound 30 as a colorless solid. LC-MS (Method A): t_{R}=2.17 min; MS (m/z) [M+H]⁺ calc. for C₄₂H₄₈N₃O₁₇S 898.27, found 898.09.

### Synthetic Example 23

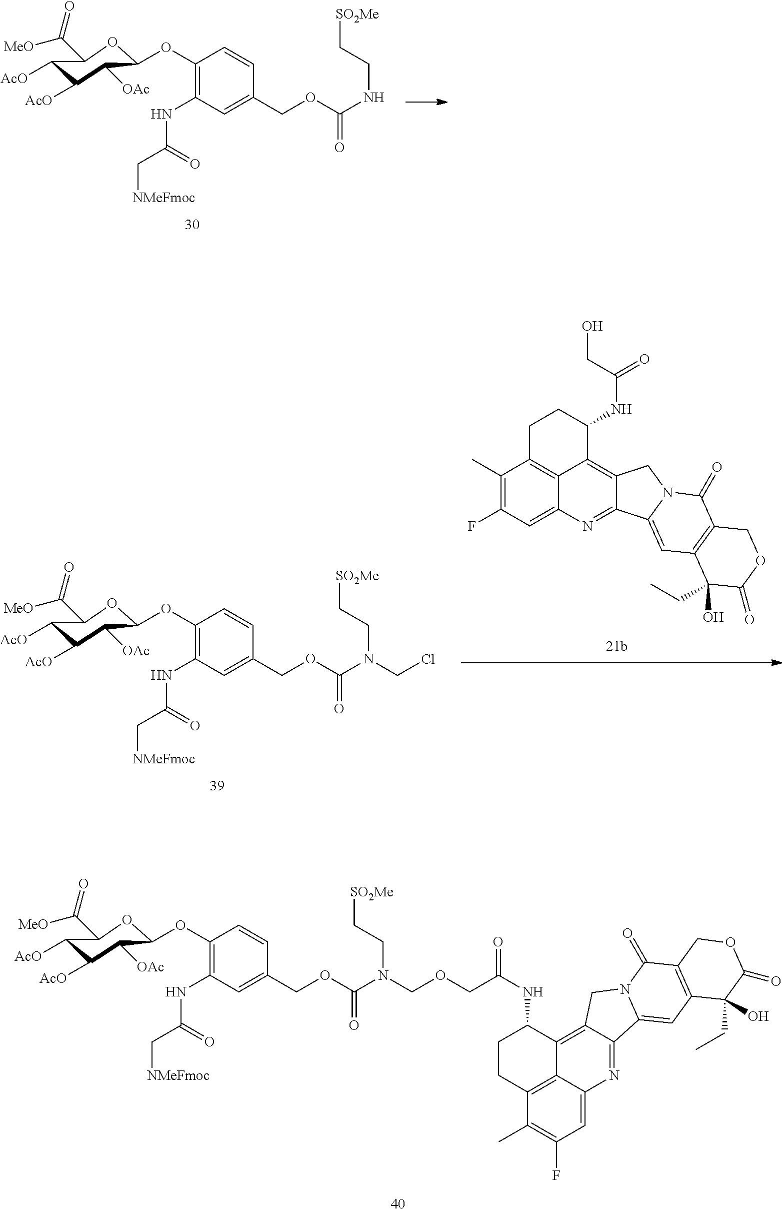

Compound 30 (200 mg, 0.223 mmol) was dissolved in DCM (2 mL). Paraformaldehyde (200 mg, 6.68 mmol) was added, followed by TMSCl (1 mL). The reaction was stirred for 15 minutes, then filtered, rinsed DCM (2×2 mL), and toluene (2 mL) was added to azeotrope the final mixture. The eluent was concentrated to afford a white solid. The crude compound 39 was used immediately in the next step.

Crude compound 39 (0.223 mmol) was dissolved in anhydrous DCM (1 mL). DIPEA (0.047 mL, 0.27 mmol) was added to the reaction. The reaction solution was added directly to the solid compound 21-b (22 mg, 0.045 mmol). The reaction was stirred for 120 minutes. The reaction was quenched with MeOH, concentrated in vacuo and purified by column chromatography 0-10% MeOH in DCM. Fractions containing the desired product and minor impurity concentrated in vacuo to afford compound 40 as a white solid (30 mg, 80% w/w, 0.021 mmol, 48%). LC-MS (Method A): t_{R}=2.25 min; MS (m/z) [M+H]⁺ calc. for C₆₉H₇₂FN₆O₂₃S 1403.44, found 1404.03.

### Synthetic Example 24

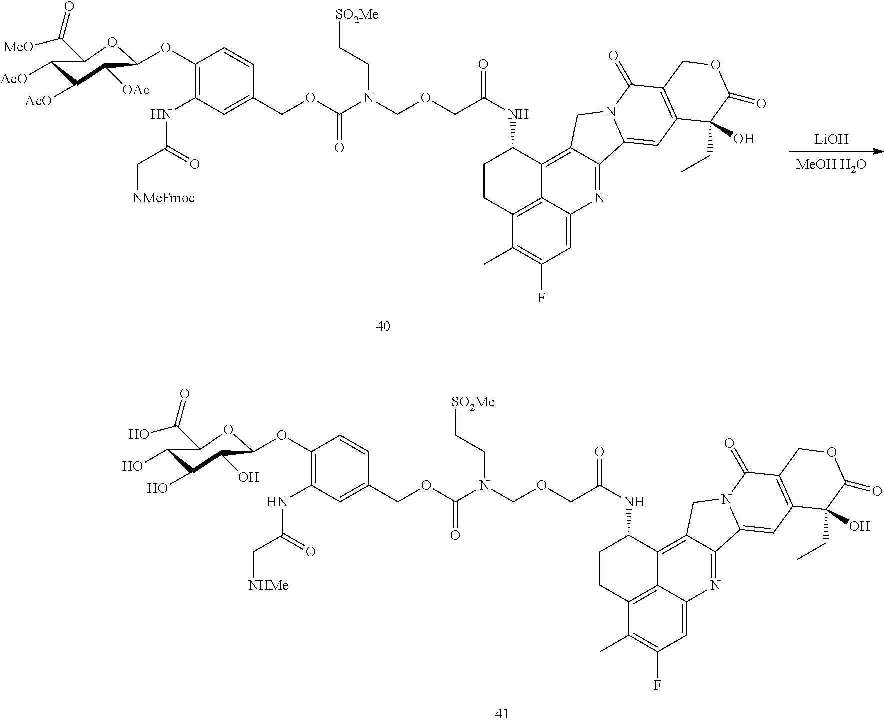

Compound 40 (30 mg, 0.021 mmol) was dissolved in MeOH (1 mL). LiOH (25 mg, 1.1 mmol) was added to the reaction, and the reaction was sonicated to aid in dissolution. The reaction was stirred for 10 minutes then H₂O (1 mL) was added. The reaction was stirred an additional 20 minutes then quenched with AcOH. The reaction was concentrated in vacuo and purified by prep-HPLC 21 mm 5-60-95% MeCN in H₂O 0.05% TFA. Fractions containing the desired product were purified to afford compound 41 as a white solid (14.5 mg, 0.0139 mmol, 65%). LC-MS (Method A): t_{R}=1.29 min; MS (m/z) [M+H]⁺ calc. for C₄₇H₅₄FN₆O₁₈S 1041.32, found 1041.24.

### Synthetic Example 25

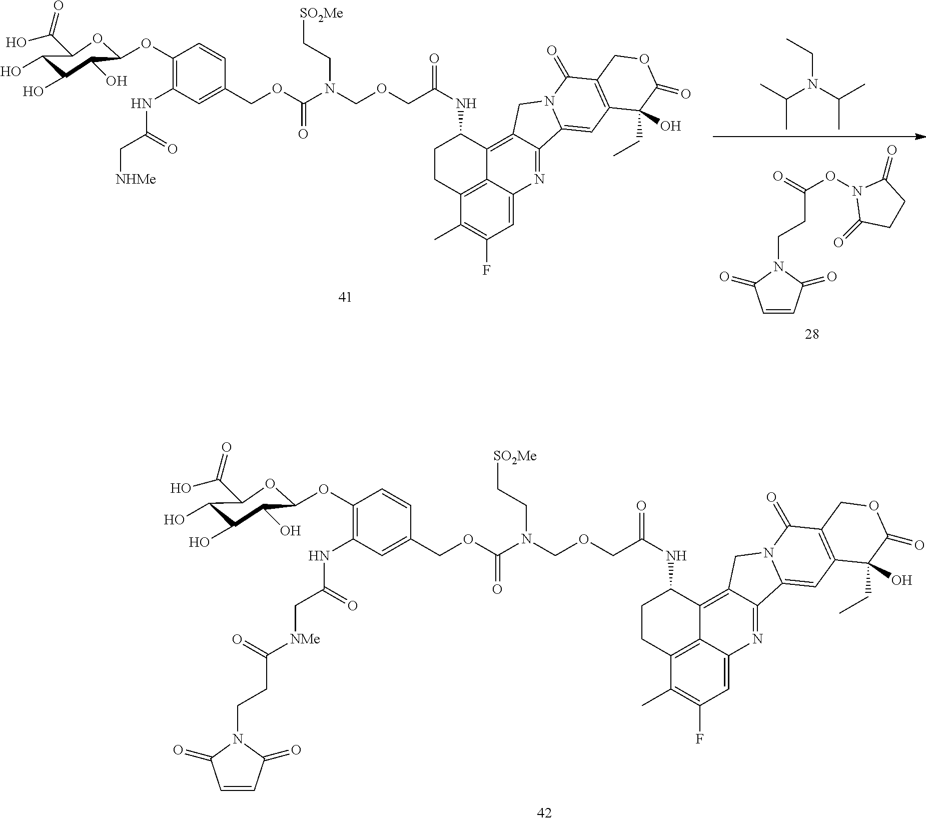

Compound 41 (14.5 mg, 0.0139 mmol) was dissolved in anhydrous DMF (0.5 mL). DIPEA (15 μL, 0.084 mmol) was added followed by 3-(maleimido)-propionic acid N-hydroxysuccinimide ester (28, 11 mg, 0.042 mmol). The reaction was stirred at room temperature for 80 minutes. The reaction was quenched with AcOH and purified by prep-HPLC 10 mm 5-60-95% MeCN in H₂O 0.05% TFA. Fractions containing the desired product were lyophilized to afford compound 42 as a yellow solid (2.24 mg, 1.88 μmol, 13%). LC-MS (Method A): t_{R}=1.49 min; MS (m/z) [M+H]⁺ calc. for C₅₄H₅₉FN₇O₂₁S 1192.35, found 1192.31.

### Synthetic Example 26

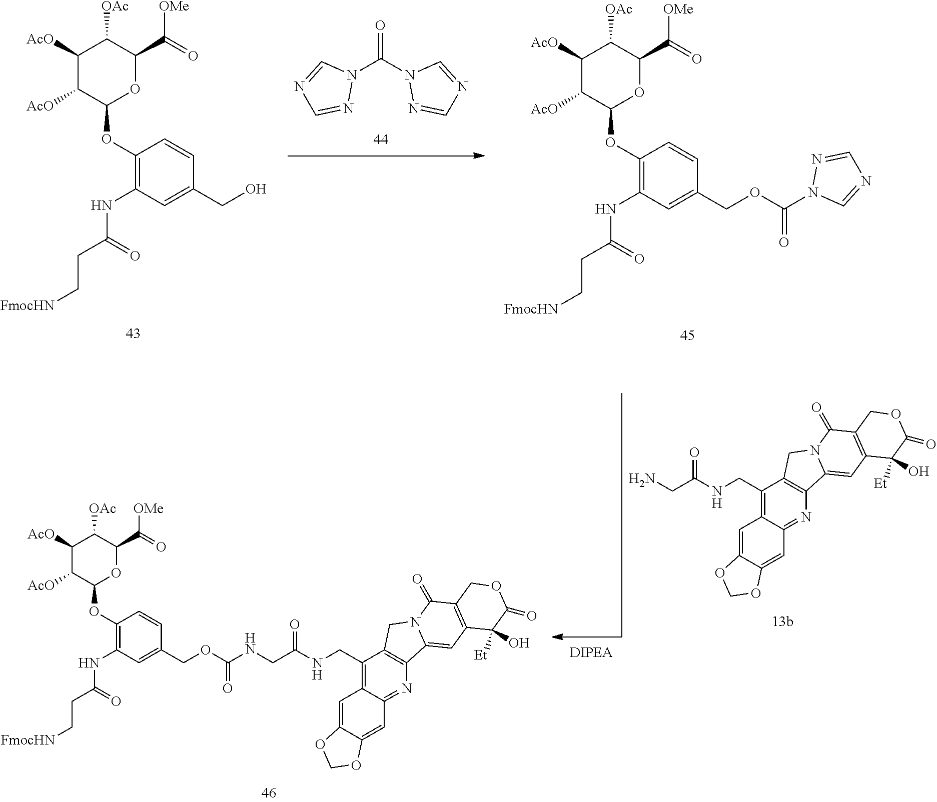

Compound 43 (200 mg, 0.267 mmol), prepared according to the procedure of Bioconjugate Chem. (2006) 17: 831-840), was dissolved in DCM (1 mL). Carbonyl ditriazole (44, 131.52 mg, 0.801 mmol) was added and the reaction was stirred for 30 minutes. Complete conversion was observed by UPLC-MS. The reaction was diluted with EtOAc (50 mL), and washed H₂O (3×50 mL). The organic was dried with MgSO₄, filtered and concentrated in vacuo to afford compound 45 as a white solid (209 mg, 0.248 mmol, 93%). LC-MS (Method D): t_{R}=2.07 min; MS (m/z) [M+H]⁺ calc. for C₄₁H₄₂N₅O₁₅ 844.27, found 844.02. The product was used in the next step without further purification.

Compound 45 (100 mg, 0.119 mmol) and compound 13b (18 mg, 0.039 mmol), referred to as H-Gly-7-MAD-MDCPT, were dissolved in DMF (0.5 mL). DIPEA (0.1 mL) was added and the reaction was stirred at room temperature. Approximately 50% conversion observed to the desired product, compound 46, with hydrolysis of compound 45 was observed after 15 minutes. Note: Compound 46 and hydrolysis product had the same retention time by UPLC-MS. Reaction was quenched AcOH and concentrated in vacuo, then purified by column chromatography 0-5% MeOH in DCM. Fractions containing compound 46 with 50% compound 13b impurity were concentrated to afford a white solid (46 mg, 50% w/w, 0.018 mmol, 46%). LC-MS (Method E): t_{R}=1.32 min; MS (m/z) [M+H]⁺ calc. for C₆₃H₆₁N₆O₂₂ 1253.38, found 1253.47.

### Synthetic Example 27

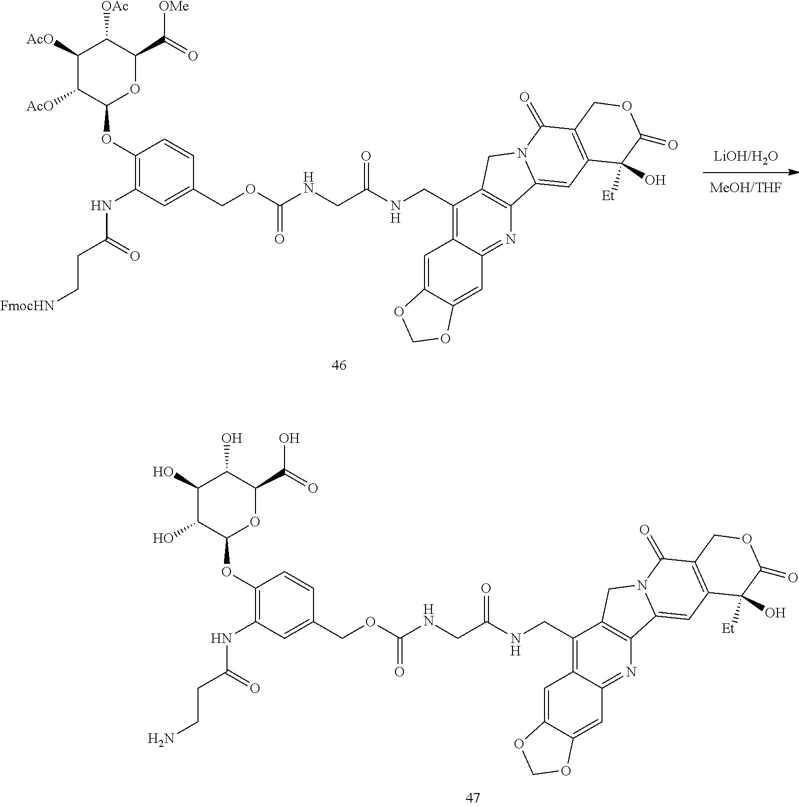

Compound 46 (0.018 mmol) was dissolved in MeOH (0.5 mL) and THF (0.5 mL). LiOH (25 mg, 1.0 mmol) was added. The reaction was sonicated to solubilize LiOH and stirred. After 10 minutes water was added (0.5 mL). Complete conversion was observed by UPLC-MS after 100 minutes. The reaction was quenched with AcOH (0.2 mL). The reaction was concentrated, then purified by Prep-HPLC using a 10 mm Max-RP with a 5-60-95 MeCN in H₂O 0.05% TFA gradient. Fractions containing the desired product compound 47 were concentrated in vacuo to afford a yellow solid (8.4 mg, 9.4 μmol, 51%). LC-MS (Method D): t_{R}=0.92 min; MS (m/z) [M+H]⁺ calc. for C₄₁H₄₃N₆O₁₇ 891.27, found 891.06.

### Synthetic Example 28

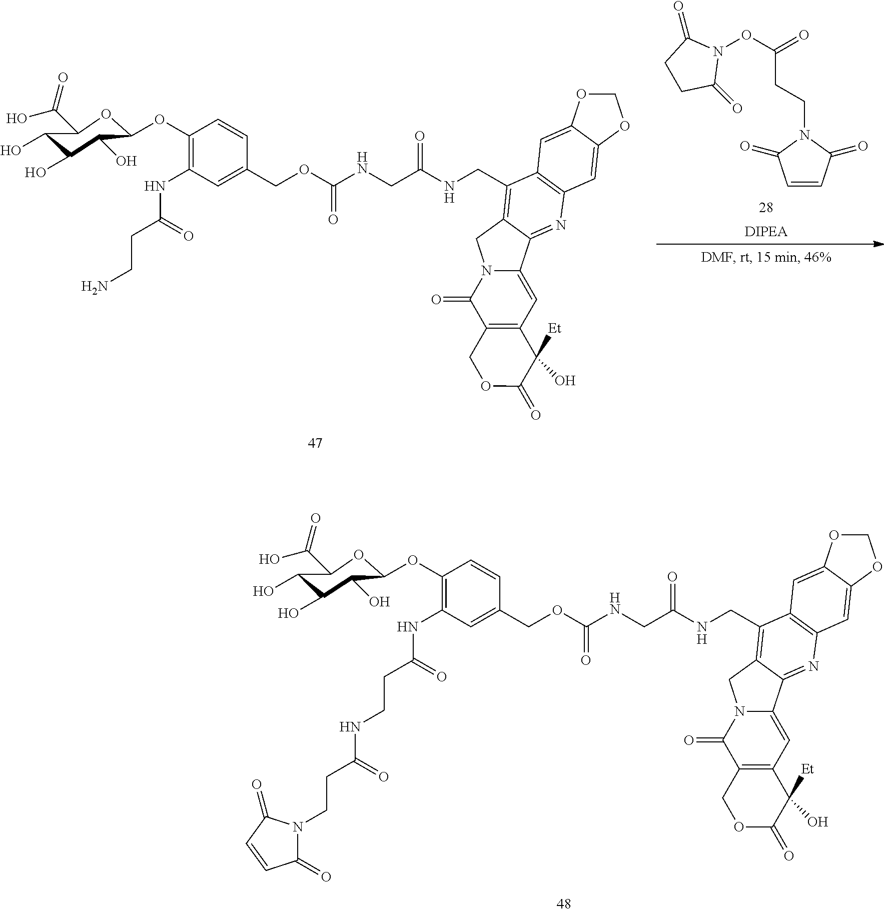

Compound 47 (8.4 mg, 9.4 μmol) was dissolved in DMF (0.2 mL). 3-(Maleimido)propionic acid N-hydroxysuccinimide ester (28, 7.5 mg, 0.028 mmol) was added. DIPEA (9 μL, 0.05 mmol) was added. The reaction was stirred for 5 minutes at which point complete conversion was observed by UPLC-MS. The reaction was quenched with AcOH (0.05 mL), the purified by Prep-HPLC 10 mm Max RP C12. Fractions containing the desired product were lyophilized to afford a yellow powder with 10% impurity. The crude lyophilized product was re-purified by Prep-HPLC 10 mm Max-RP C12 fractions containing the desired product were lyophilized to afford compound 48 as a yellow solid (1.33 mg, 1.28 μmol, 13.5%). LC-MS (Method D): t_{R}=1.08 min; MS (m/z) [M+H]⁺ calc. for C₄₈H₄₈N₇O₂₀ 1042.30, found 1042.19.

### Synthetic Example 29

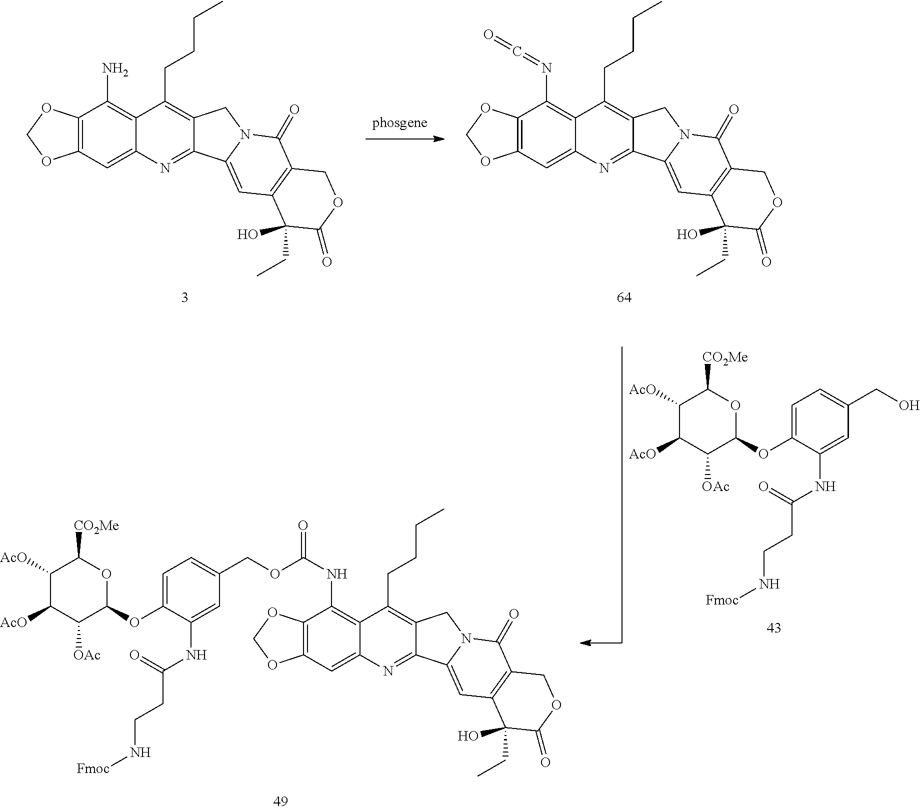

A flame dried flask was charged with compound 3 (30 mg, 65 μmol) and flushed with N₂. Anhydrous DCM (3.25 mL) was added followed by phosgene (20% in toluene, 1.2 mL). The reaction was capped and stirred for 24 h. Isocyanate formation was confirmed by spiking reaction mixture into MeOH and observing methyl carbamate adduct. LC-MS (Method A): t_{R}=1.58 min, MS m/z (ES+) found 522.32. The reaction was stirred to dryness under N₂ stream, placed under high vacuum for 1 h to provide compound 64, which was carried forward without further purification.

Compound 43 (103 mg, 138 μmol), prepared according to the procedure of Bioconjugate Chem. (2006) 17: 831-840, was solubilized in anhydrous DMF (1.5 mL) and added to a flask charged with compound 64 (32 mg, 65 μmol). The reaction was stirred under N₂ for 24 h then concentrated in vacuo to dryness. Crude mixture was loaded onto a 1 mM chromatotron plate and eluted with DCM/MeOH (1%, 2%, 3% MeOH gradient) to yield compound 49 (25 mg, 31%). LC-MS (Method A): t_{R}=2.23 min; MS (m/z) calculated 1238.22 (M+H)⁺, found 1238.40.

### Synthetic Example 30

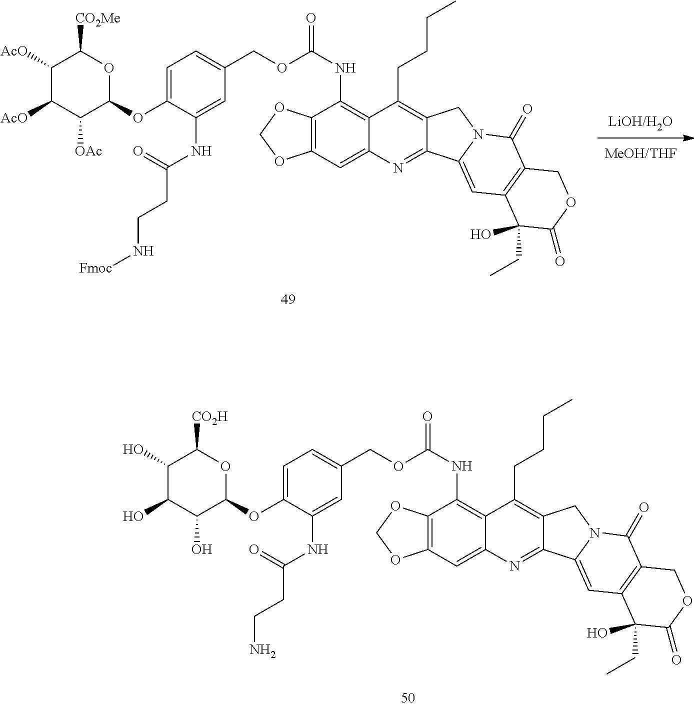

Compound 49 (3, 41 mg, 33 μmol) was solubilized in MeOH (1.1 mL) and THF (1.1 mL) then cooled to 0° C. LiOH monohydrate (14 mg, 333 μmol) was taken up in H₂O (1.1 mL) then added to the reaction dropwise with stirring. The reaction was allowed to warm to RT and stopped after 4.5 h. MeOH and THF were removed in vacuo, DMSO was added to solubilize, then the reaction was purified by preparative HPLC to provide compound 50 (9 mg, 31%). LC-MS (Method A): t_{R}=1.16 min; MS m/z (ES+) found 876.23.

### Synthetic Example 31

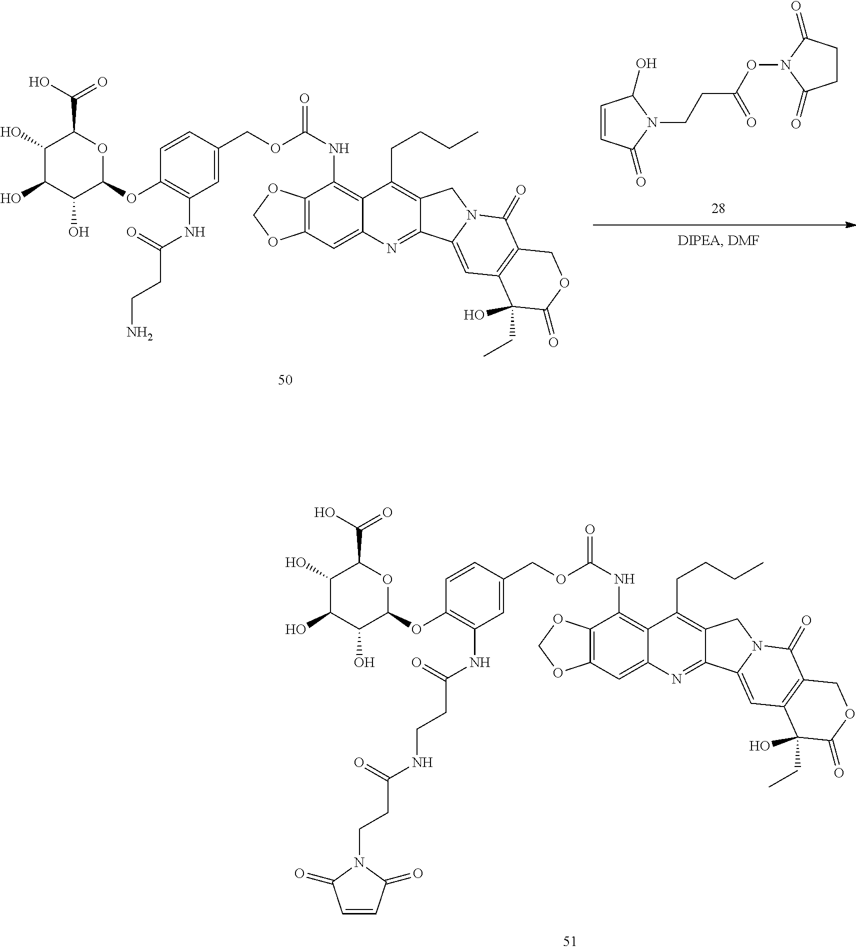 [SMILES: CCCCc1c2c(nc3cc4c(c(NC(=O)OCc5ccc(O[C@@H]6O[C@H](C(=O)O)[C@@H](O)[C@H](O)[C@H]6O)c(NC(=O)CCN)c5)c13)OCO4)-c1cc3c(c(=O)n1C2)COC(=O)[C@]3(O)CC ; O=C(CCN1C(=O)C=CC1O)ON1C(=O)CCC1=O ; CCCCc1c2c(nc3cc4c(c(NC(=O)OCc5ccc(O[C@@H]6O[C@H](C(=O)O)[C@@H](O)[C@H](O)[C@H]6O)c(NC(=O)CCNC(=O)CCN6C(=O)C=CC6=O)c5)c13)OCO4)-c1cc3c(c(=O)n1C2)COC(=O)[C@]3(O)CC]

3-(Maleimido)propionic acid N-hydroxysuccinimide ester (28) was dissolved in anhydrous DMF and added to compound 50 to a final concentration of 30 mM, followed by addition of DIPEA. The reaction was monitored by LC-MS. Upon completion, the solution was neutralized with acetic acid, concentrated, and then purified by preparative HPLC to yield compound 51. LC-MS (Method A): t_{R}=1.36 min, MS (m/z) calculated 1026.96 (M+H)⁺, found 1027.21.

### Synthetic Example 32

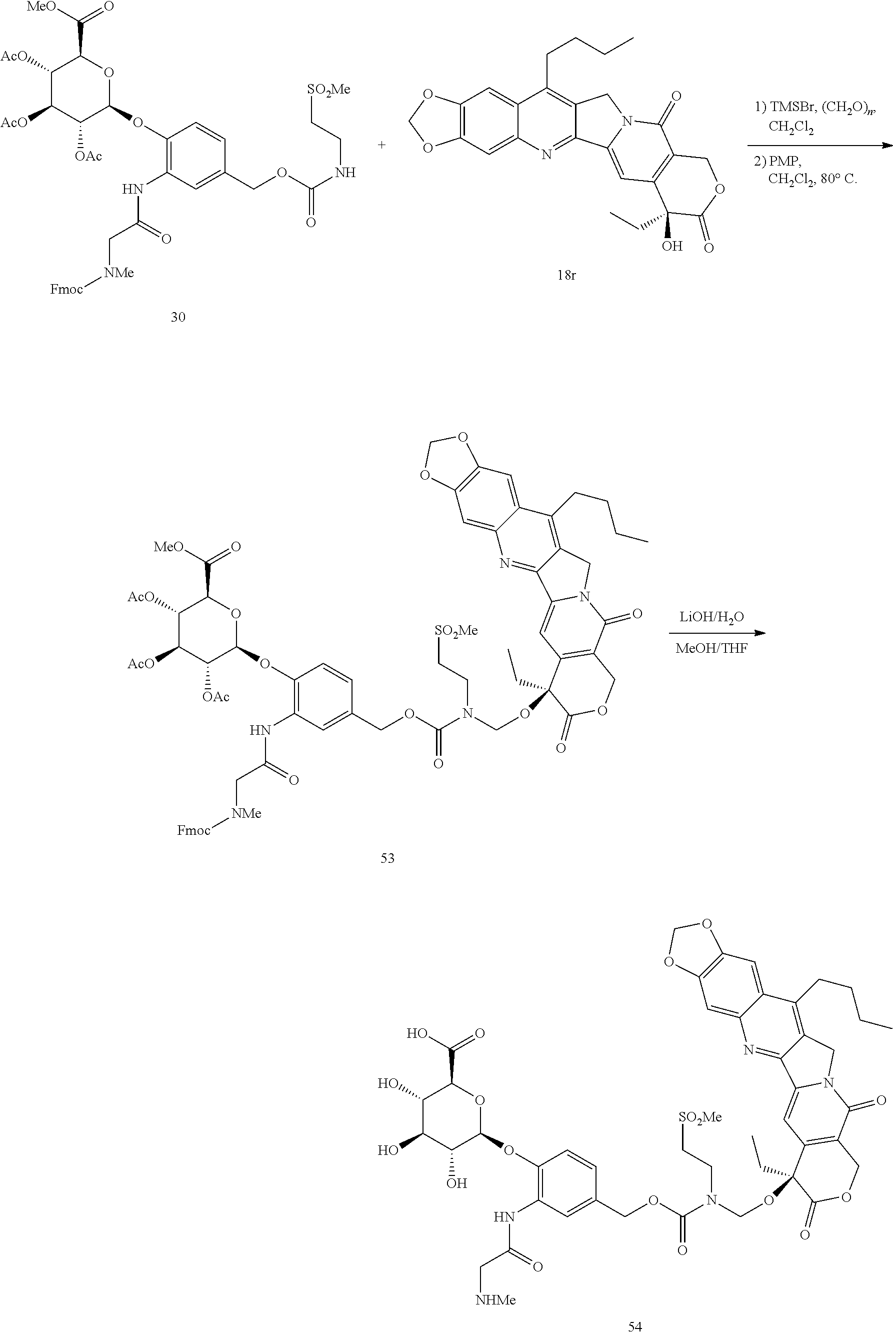

In an oven dried flask, compound 30 (162 mg, 180 μmol), prepared as described in Example 21, was dissolved in anhydrous dichloromethane (1 mL), followed by the addition of paraformaldehyde (10.8 mg, 0.36 mmol) and TMSBr (250 μL, 1.40 mmol). The solution was stirred at room temperature for 10 min with monitoring by quenching with methanol and observing the formation of the methanol adduct by LC-MS. The reaction was filtered, washed with anhydrous toluene and dichloromethane, and dried in vacuo for 3 cycles to yield the crude product, which was used in the subsequent reaction without further purification. The crude compound was re-dissolved in anhydrous dichloromethane and added to compound 18r (32.3 mg, 72 μmol), prepared according to the procedure in Bioconjugate Chem. (2009) 20: 1242-1250, followed by 1,2,2,6,6-pentamethylpiperidine (PMP, 200 μL, 0.96 mmol). The reaction was capped, microwaved at 80° C. for 2 h, and monitored by LC-MS. Upon completion, the solvent was removed and purified by preparative HPLC to yield the desired product, compound 53. MS (m/z) calculated 1358.39 (M+H)⁺, found 1358.03.

Crude compound 53 was dissolved in MeOH and THF, and cooled to 0° C. LiOH in H₂O was slowly added to the reaction flask to a final concentration of 10 mM. The reaction was warmed to room temperature, and monitored by LC-MS. Upon completion, the solution was neutralized with acetic acid, concentrated, and then purified by preparative HPLC to yield compound 54. MS (m/z) calculated 996.01 (M+H)⁺, found 996.36.

### Synthetic Example 33

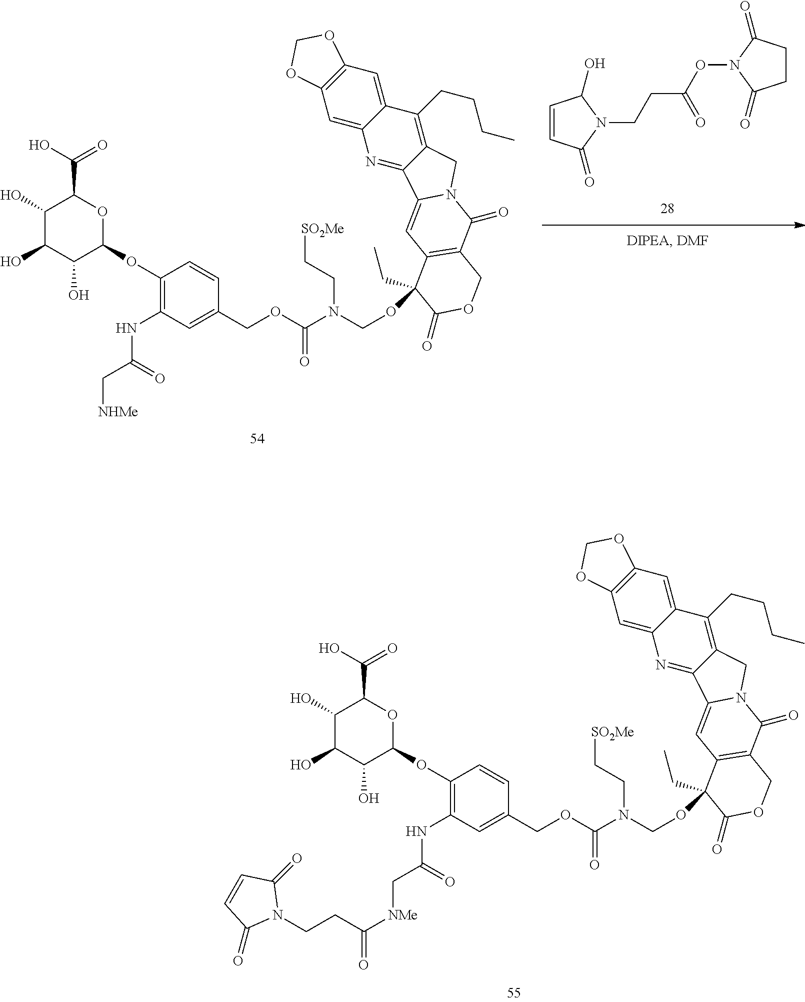

3-(Maleimido)propionic acid N-hydroxysuccinimide ester (28) was dissolved in anhydrous DMF and added to compound 54 to a final concentration of 30 mM, followed by addition of DIPEA. The reaction was monitored by LC-MS. Upon completion, the solution was neutralized with acetic acid, concentrated, and then purified by preparative HPLC to yield compound 55. LC-MS (Method A): t_{R}=1.75 min, MS (m/z) calculated 1147.13 (M+H)⁺, found 1147.02.

### Synthetic Example 34

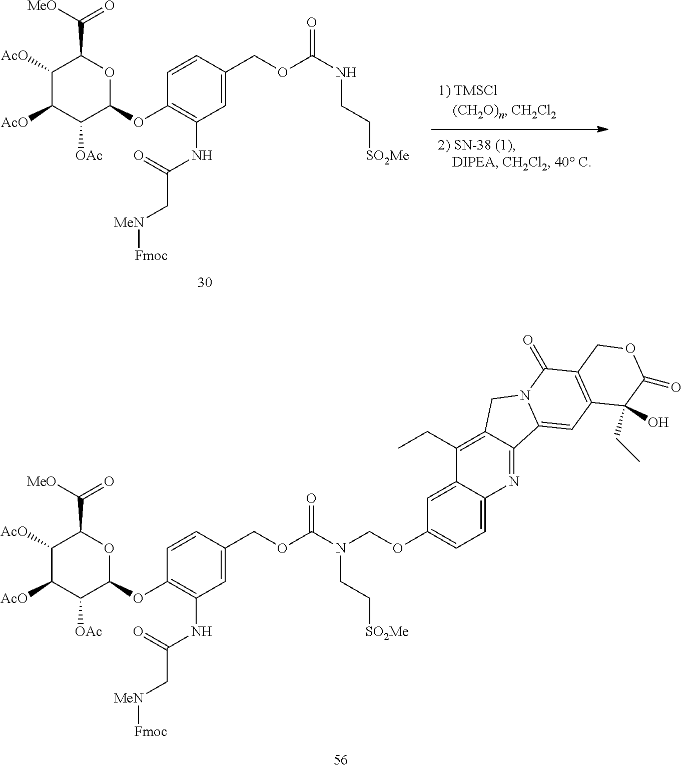

In an oven dried flask compound 30 (1235 mg, 1.38 mmol), prepared as described in Example 21, was dissolved in anhydrous dichloromethane (5 mL), followed by the addition of paraformaldehyde (13.8 mg, 0.46 mmol) and TMSCl (1.0 mL, 7.88 mmol). The solution was stirred at room temperature for 30 min with monitoring by quenching with methanol and observing the formation of the methanol adduct by LC-MS. MS (m/z) calculated for the methanol adduct 942.29 (M+H)⁺, found 942.28. The reaction was filtered, washed with anhydrous toluene and dichloromethane, and dried in vacuo for 3 cycles. The crude product was dissolved in anhydrous dichloromethane (6 mL) and DIPEA (359 μL, 2.06 mmol), and added to the flask containing SN-38 (compound 1, 90 mg, 0.23 mmol). The reaction was capped and stirred at 40° C. for 18 h. The reaction mixture was purified over silica via Biotage flash column chromatography (CH₂Cl₂/MeOH, 0-10%) to yield compound 56. MS (m/z) calculated 1302.40 (M+H)⁺, found 1302.36.

### Synthetic Example 35

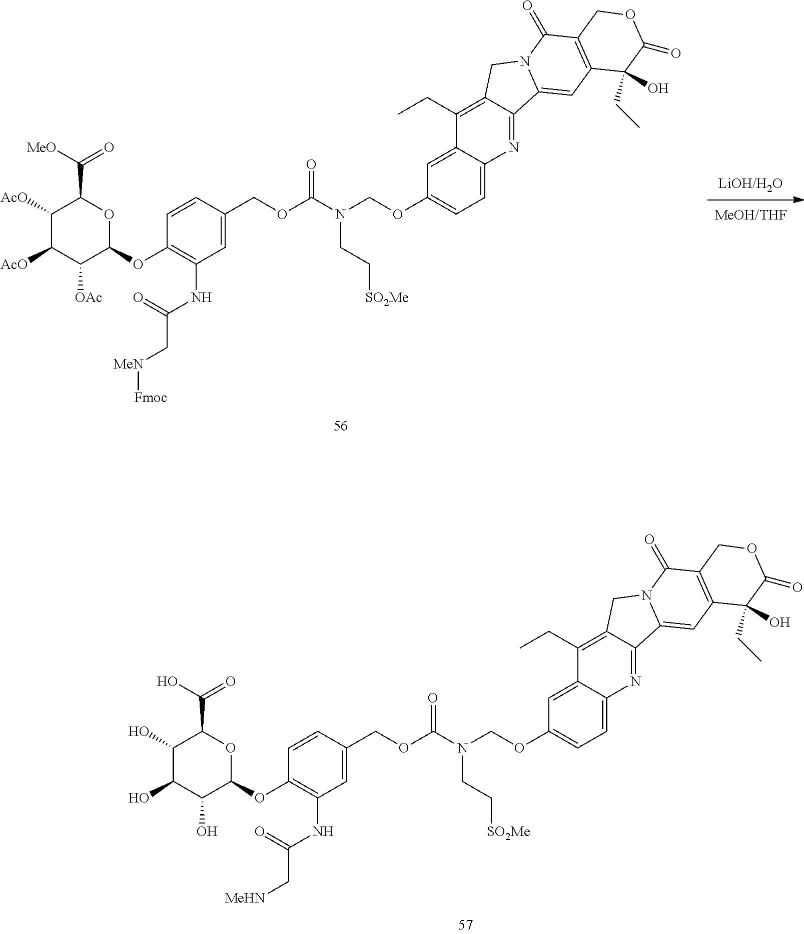

Compound 56 (282.6 mg, 0.22 mmol) was dissolved in THF and MeOH and cooled to 0° C. in an ice bath. LiOH (91.1 mg, 2.17 mmol) was dissolved in H₂O and added dropwise. The reaction was stirred at room temperature and was complete within 45 min. The reaction was neutralized with acetic acid, concentrated, and directly purified by preparative HPLC to yield compound 57.

### Synthetic Example 36

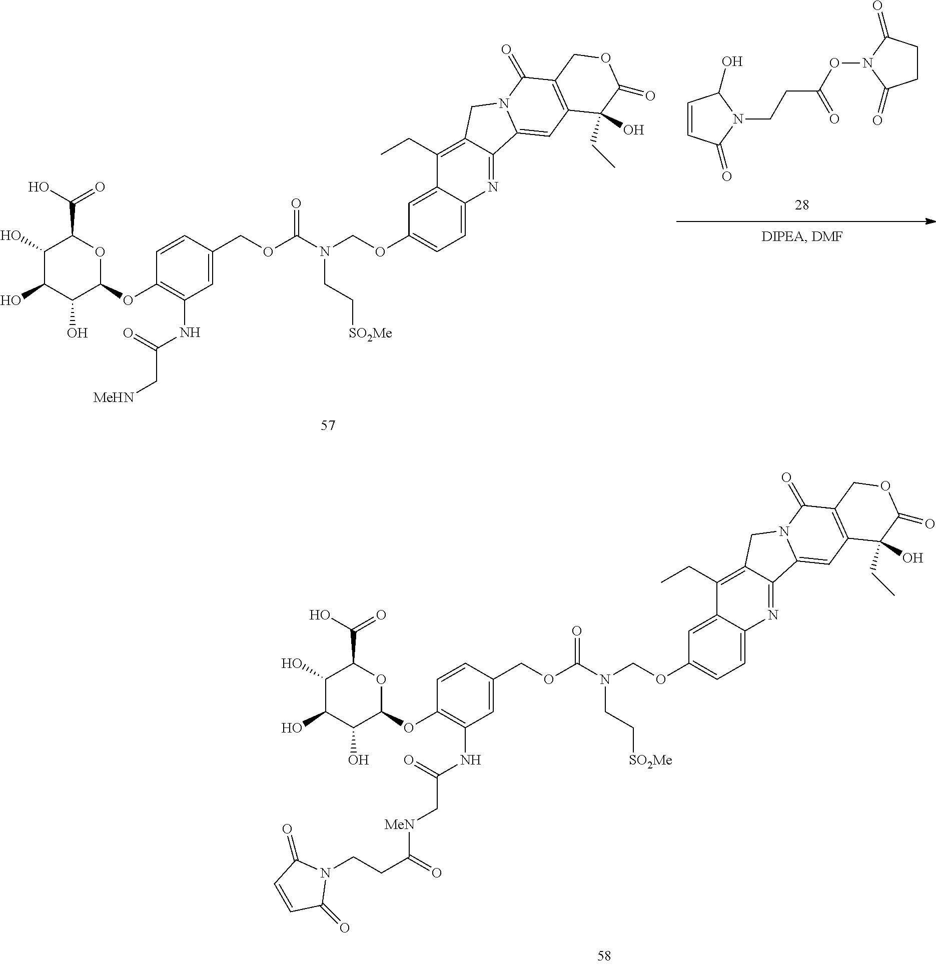

3-(Maleimido)propionic acid N-hydroxysuccinimide ester (28) was dissolved in DMF and DIPEA, and added to compound 57. The reaction was stirred for 3 h until completion as monitored by LC-MS. The reaction mixture was neutralized with acetic acid and directly purified by preparative HPLC to yield compound 58. LC-MS (Method A): t_{R}=1.40 min; MS (m/z) calculated 1091.31 (M+H)⁺, found 1091.47.

### Synthetic Example 37

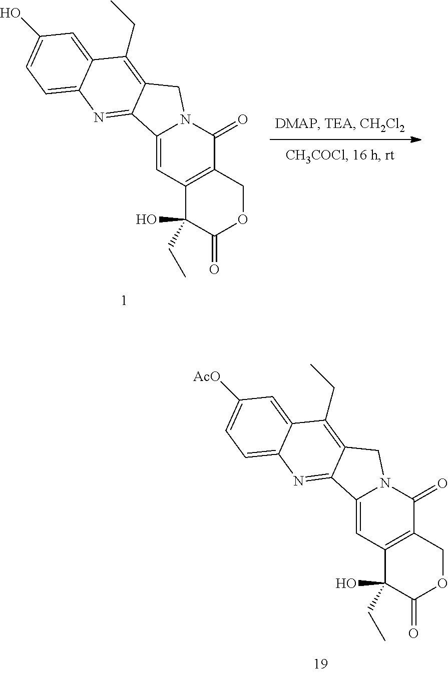 [SMILES: CCc1c2c(nc3ccc(O)cc13)-c1cc3c(c(=O)n1C2)COC(=O)[C@]3(O)CC ; CCc1c2c(nc3ccc(OC(C)=O)cc13)-c1cc3c(c(=O)n1C2)COC(=O)[C@]3(O)CC]

SN-38 (1, 76.0 mg, 0.19 mmol, purchased from MedChemExpress) was dissolved in dichloromethane, followed by addition of triethylamine (128 μL, 0.92 mmol) and DMAP (2.60 mg, 0.02 mmol). Mixture was cooled to 0° C. in an ice bath, followed by dropwise addition of acetyl chloride (15.9 μL, 0.22 mmol). The reaction mixture was stirred at room temperature for 16 h. The reaction was diluted with dichloromethane, washed with saturated NH₄Cl, water, and brine. The organic phase was then dried over MgSO₄, filtered, concentrated and purified over silica via Biotage flash column chromatography (CH₂Cl₂/MeOH 0-15%) to provide compound 19 (Ac-SN-38). MS (m/z) calculated 435.15 (M+H)⁺, found 435.07.

### Synthetic Example 38

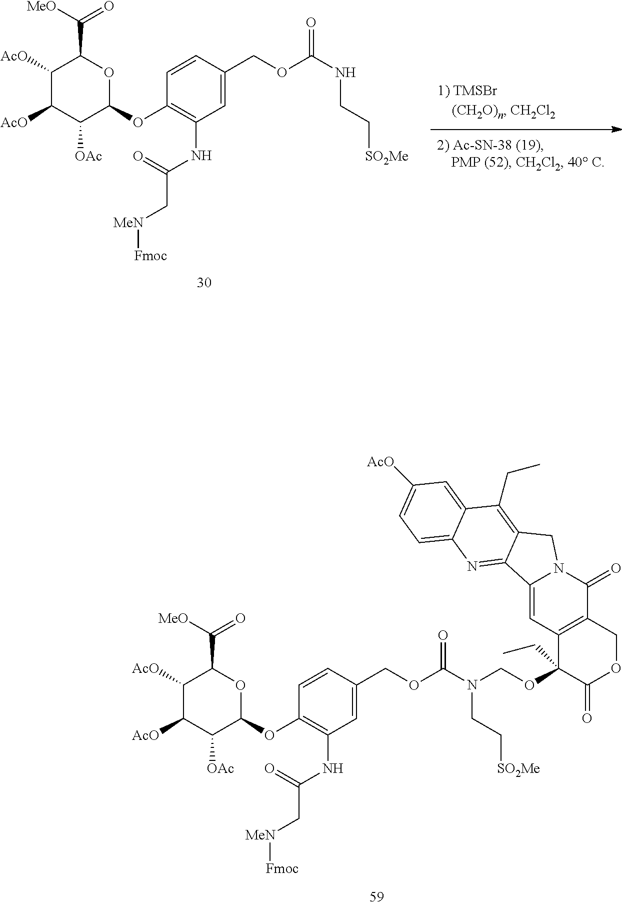

In an oven dried flask, Compound 30 (1.12 g, 1.24 mmol) was dissolved in anhydrous dichloromethane (5 mL), followed by the addition of paraformaldehyde (12.4 mg, 0.41 mmol) and TMSBr (300 μL, 1.68 mmol). The solution was stirred at room temperature for 10 min with monitoring by quenching with methanol and observing the formation of the methanol adduct by LC-MS. The reaction was filtered, washed with anhydrous toluene and dichloromethane, and dried in vacuo for 3 cycles. The crude product was dissolved in anhydrous dichloromethane and added to compound 19 (90.0 mg, 0.21 mmol), followed by 1,2,2,6,6-pentamethylpiperidine (3.90 mL, 1.86 mmol). The reaction was capped and stirred at 40° C. for 18 h. The reaction mixture was purified over silica via Biotage flash column chromatography (CH₂Cl₂/MeOH, 0-10%) to yield compound 59. MS (m/z) calculated 1344.41 (M+H)⁺, found 1344.46.

### Synthetic Example 39

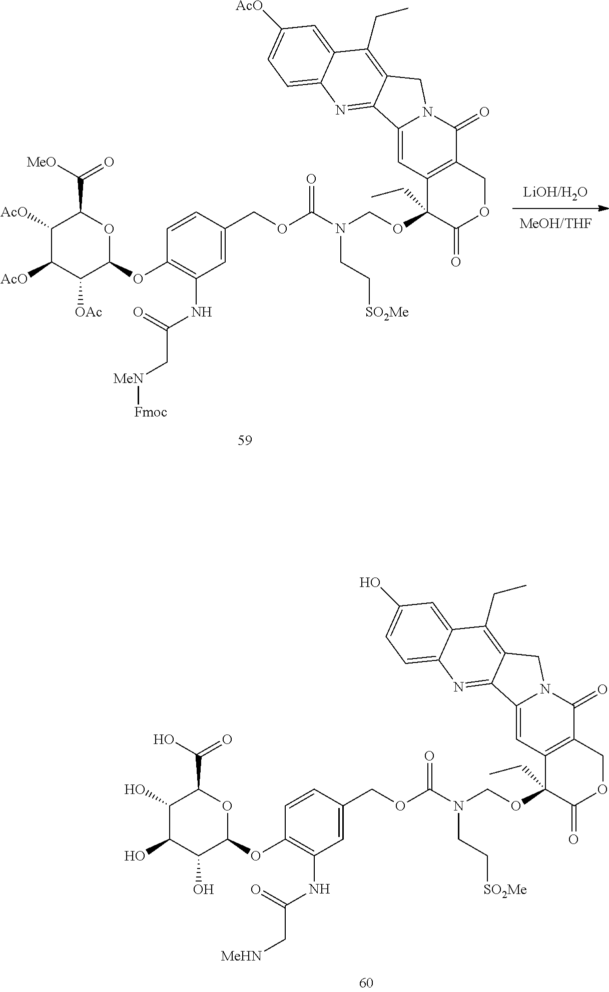

Compound 59 (290.0 mg, 0.22 mmol) was dissolved in THF and MeOH and cooled to 0° C. in an ice bath. LiOH (90.5 mg, 2.16 mmol) was dissolved in H₂O and added dropwise. The reaction was stirred at room temperature and was complete within 45 min. The reaction was neutralized with acetic acid, concentrated, and directly purified by preparative HPLC to yield the deprotected intermediate compound 60.

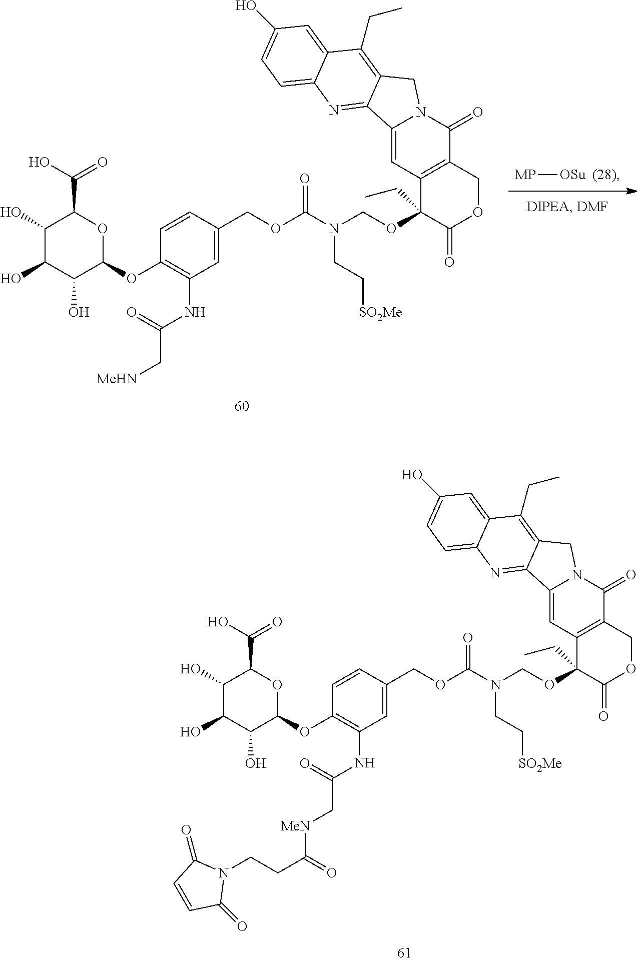

3-(Maleimido)propionic acid N-hydroxysuccinimide ester (28), referred to as MP-OSu, was dissolved in DMF and DIPEA, and added to compound 60. The reaction was stirred for 3 h until completion as monitored by LC-MS. The reaction mixture was neutralized with acetic acid and directly purified by preparative HPLC to yield compound 61. LC-MS (Method A): t_{R}=1.42 min, MS (m/z) calculated 1091.31 (M+H)⁺, found 1091.31.

### Synthetic Example 40

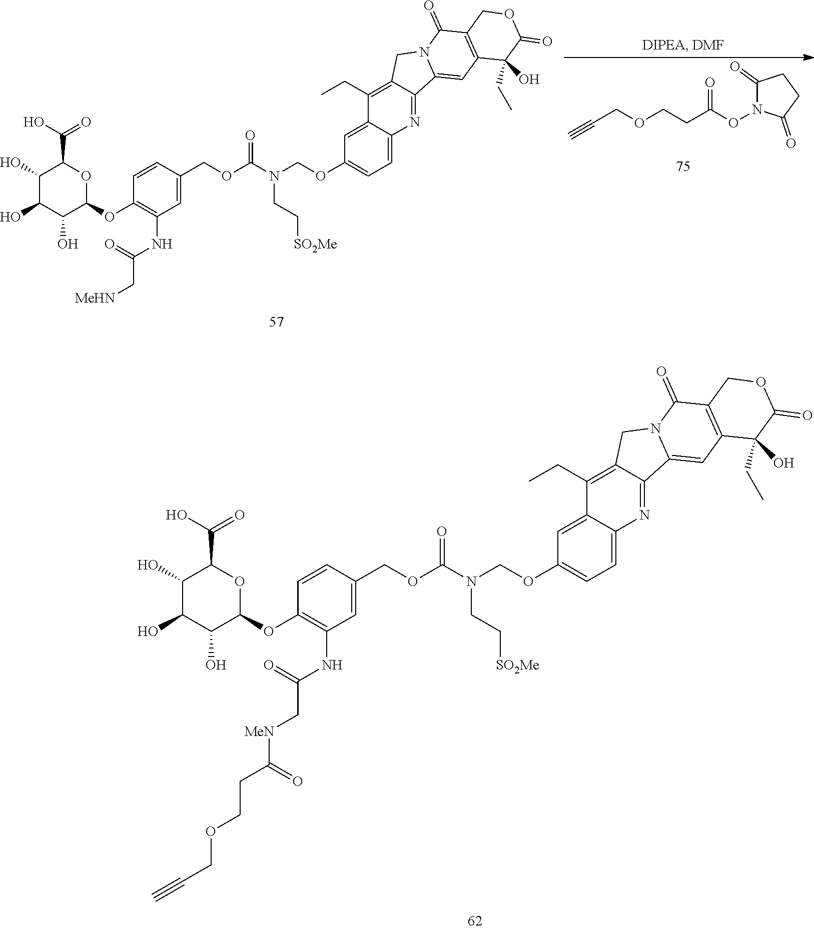 [SMILES: CCc1c2c(nc3ccc(OCN(CCS(C)(=O)=O)C(=O)OCc4ccc(O[C@@H]5O[C@H](C(=O)O)[C@@H](O)[C@H](O)[C@H]5O)c(NC(=O)CNC)c4)cc13)-c1cc3c(c(=O)n1C2)COC(=O)[C@]3(O)CC ; C#CCOCCC(=O)ON1C(=O)CCC1=O ; C#CCOCCC(=O)N(C)CC(=O)Nc1cc(COC(=O)N(CCS(C)(=O)=O)COc2ccc3nc4c(c(CC)c3c2)Cn2c-4cc3c(c2=O)COC(=O)[C@]3(O)CC)ccc1O[C@@H]1O[C@H](C(=O)O)[C@@H](O)[C@H](O)[C@H]1O]

Compound 75 (3 eq), purchased from Click Chemistry Tools (CAS:1174157-65-3), was dissolved in DMF and DIPEA, and added to compound 57. The reaction was stirred for 3 h until completion as monitored by LC-MS. The reaction mixture was neutralized with acetic acid and directly purified by preparative HPLC to yield compound 62. LC-MS (Method A): t_{R}=1.43 min, MS (m/z) calculated 1050.06 (M+H)⁺, found 1050.07.

### Synthetic Example 41

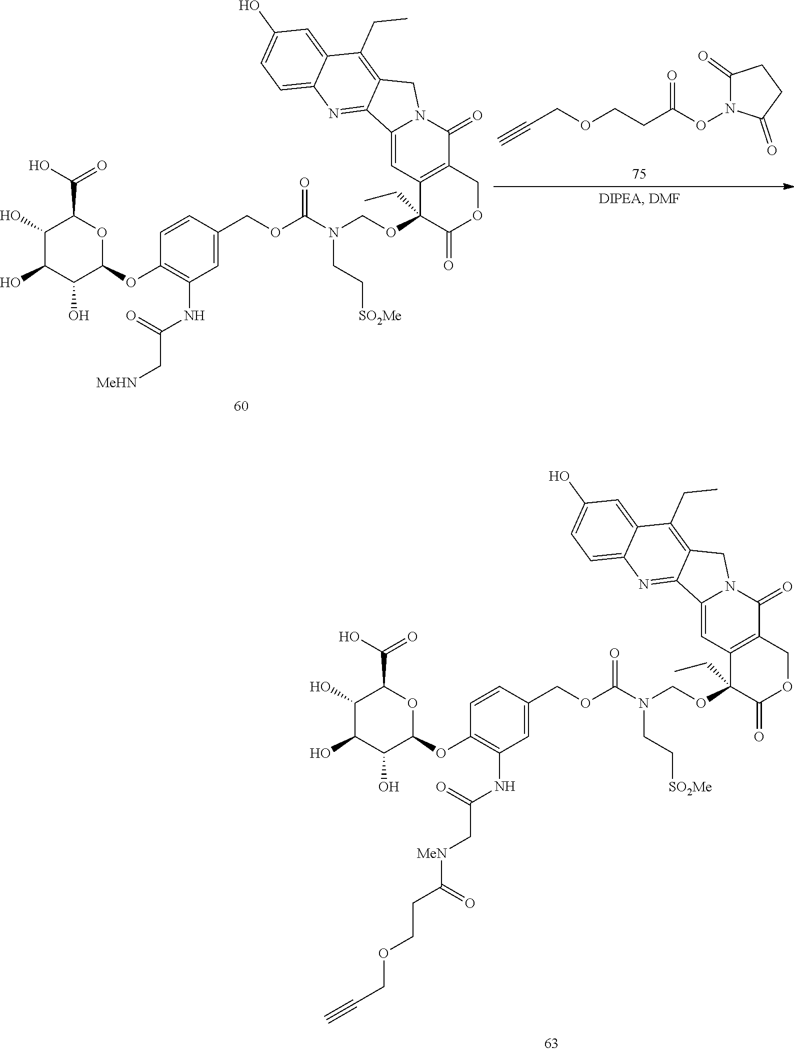

Compound 63 was prepared according to the procedure of Example 37. LC-MS (Method A); t_{R}=1.44 min; MS (m/z) calculated 1050.06 (M+H)⁺, found 1050.

### Synthetic Example 41

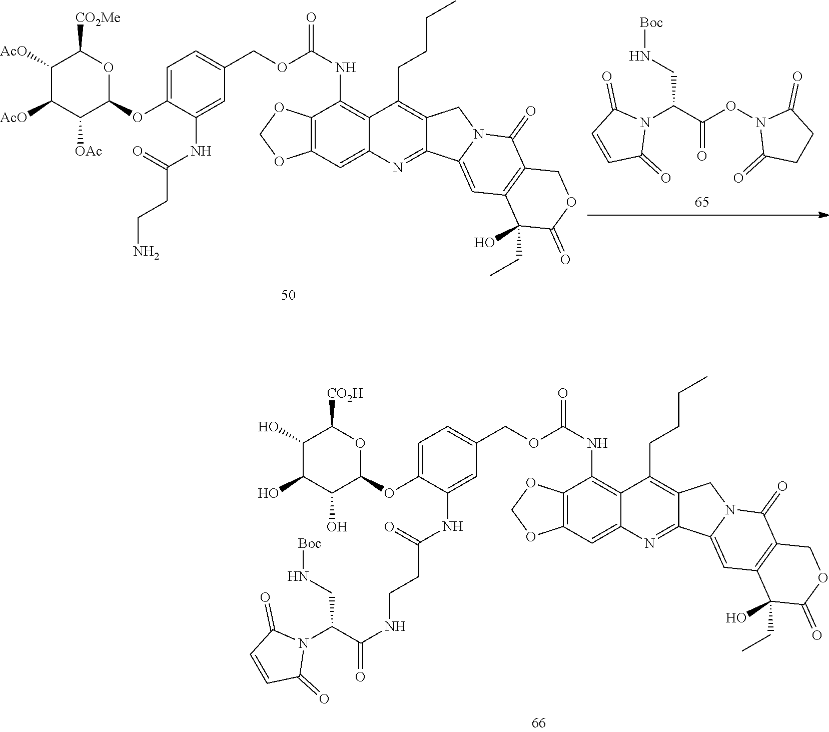 [SMILES: CCCCc1c2c(nc3cc4c(c(NC(=O)OCc5ccc(O[C@@H]6O[C@H](C(=O)OC)[C@@H](OC(C)=O)[C@H](OC(C)=O)[C@H]6OC(C)=O)c(NC(=O)CCN)c5)c13)OCO4)-c1cc3c(c(=O)n1C2)COC(=O)[C@]3(O)CC ; CC(C)(C)OC(=O)NC[C@H](C(=O)ON1C(=O)CCC1=O)N1C(=O)C=CC1=O ; CCCCc1c2c(nc3cc4c(c(NC(=O)OCc5ccc(O[C@@H]6O[C@H](C(=O)O)[C@@H](O)[C@H](O)[C@H]6O)c(NC(=O)CCNC(=O)[C@@H](CNC(=O)OC(C)(C)C)N6C(=O)C=CC6=O)c5)c13)OCO4)-c1cc3c(c(=O)n1C2)COC(=O)[C@]3(O)CC]

Compound 65 (6 mg, 16 μmol), referred to as mDPR(Boc)-OSu, prepared according to procedure of Nature Biotechnology (2014) 32: 1059-1065), was dissolved in anhydrous DMF (0.25 mL) and added to flask containing compound 50 (9 mg, 10 μmol), prepared according to the procedure of Example 30. The reaction was stirred as DIPEA (9 μL) was added, and the reaction was complete in 1.5 h. The reaction was then quenched with AcOH (9 μL), diluted in DMSO, then purified by preparative HPLC to provide compound 66 (7 mg, 61%). LC-MS (Method A): t_{R}=1.57 min; MS m/z (ES+) found 1143.51.

### Synthetic Example 43

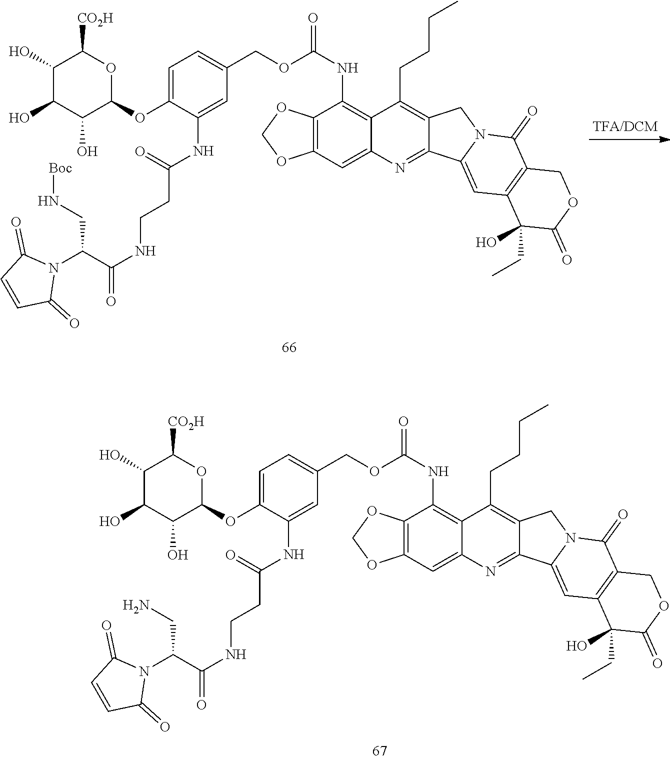 [SMILES: CCCCc1c2c(nc3cc4c(c(NC(=O)OCc5ccc(O[C@@H]6O[C@H](C(=O)O)[C@@H](O)[C@H](O)[C@H]6O)c(NC(=O)CCNC(=O)[C@@H](CNC(=O)OC(C)(C)C)N6C(=O)C=CC6=O)c5)c13)OCO4)-c1cc3c(c(=O)n1C2)COC(=O)[C@]3(O)CC ; CCCCc1c2c(nc3cc4c(c(NC(=O)OCc5ccc(O[C@@H]6O[C@H](C(=O)O)[C@@H](O)[C@H](O)[C@H]6O)c(NC(=O)CCNC(=O)[C@@H](CN)N6C(=O)C=CC6=O)c5)c13)OCO4)-c1cc3c(c(=O)n1C2)COC(=O)[C@]3(O)CC]

Compound 66 (7 mg, 6 μmol) was stirred at 0° C. in anhydrous DCM (0.54 mL) followed by dropwise addition of TFA (0.06 mL). The reaction was complete in 2.5 h. The reaction was diluted in DMSO, DCM removed in vacuo, then purified by preparative HPLC to provide compound 67 (4 mg, 64%). LC-MS (Method A): t_{R}=1.18 min; MS m/z (ES+) found 1041.22.

### Synthetic Example 44

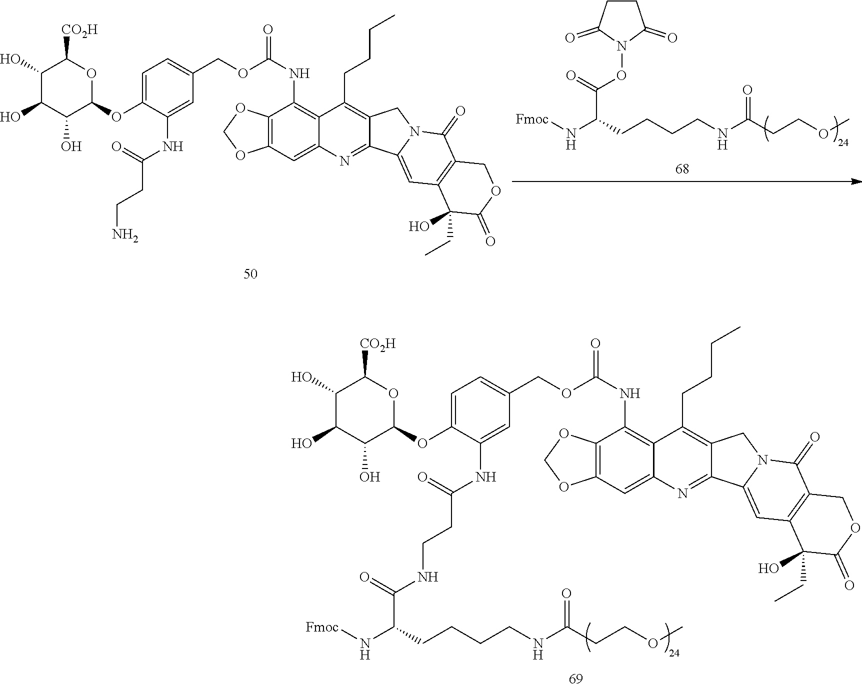

Compound 68 (86 mg, 56 μmol), referred to as Fmoc-Lys(PEG24)-OSu and prepared according to the procedure of WO 2017165851 was taken up into anhydrous DMF (0.93 mL) and added to a flask charged with compound 50 (32 mg, 37 μmol), prepared according to the procedure of Example 30. DIPEA (32 μL) was added, the reaction was stirred for 1 h, the diluted in DMSO and purified by preparative HPLC to provide compound 69 (25 mg, 29%). LC-MS (Method A): t_{R}=1.68 min; MS m/z (ES+) found 1163.50 (½ mass).

### Synthetic Example 45

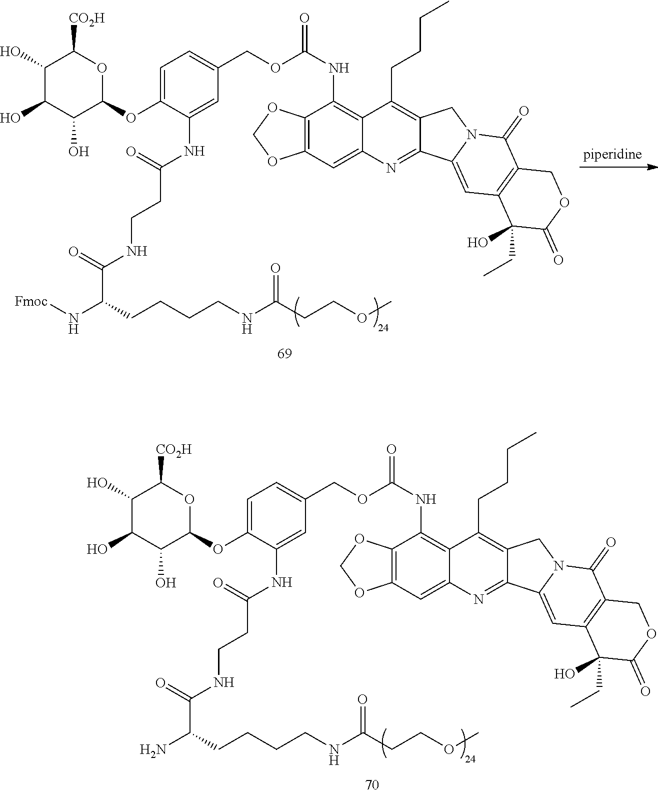

Compound 69 (10, 25 mg, 11 μmol) was taken up in 20% piperidine in DMF (0.55 mL) and stirred for 1 h. The reaction was then diluted in DMSO and purified by preparative HPLC to provide compound 70 (22 mg, 95%). LC-MS (Method A): t_{R}=1.31 min, MS m/z (ES+) found 1052.41 (½ mass).

### Synthetic Example 46

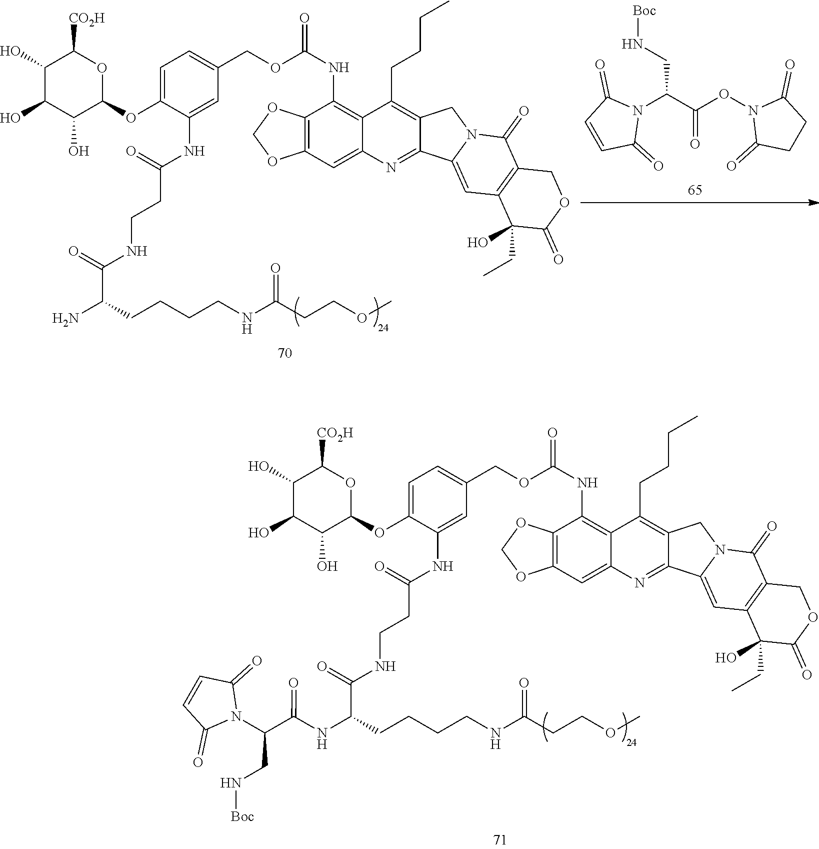

Compound 65 (8 mg, 21 μmol), referred to as mDPr(Boc)-OSu was solubilized in DMF (0.2 mL) then transferred to a flask containing compound 70 (11, 22 mg, 10 μmol) followed by DIPEA (9 μL). The reaction was stirred for 3 h, quenched with AcOH (9 μL), diluted in DMSO, and purified by preparative HPLC to provide compound 71 (12 mg, 51%). LC-MS (Method A): t_{R}=1.58 min; MS m/z (ES+) found 1185.52 (½ mass).

### Synthetic Example 47

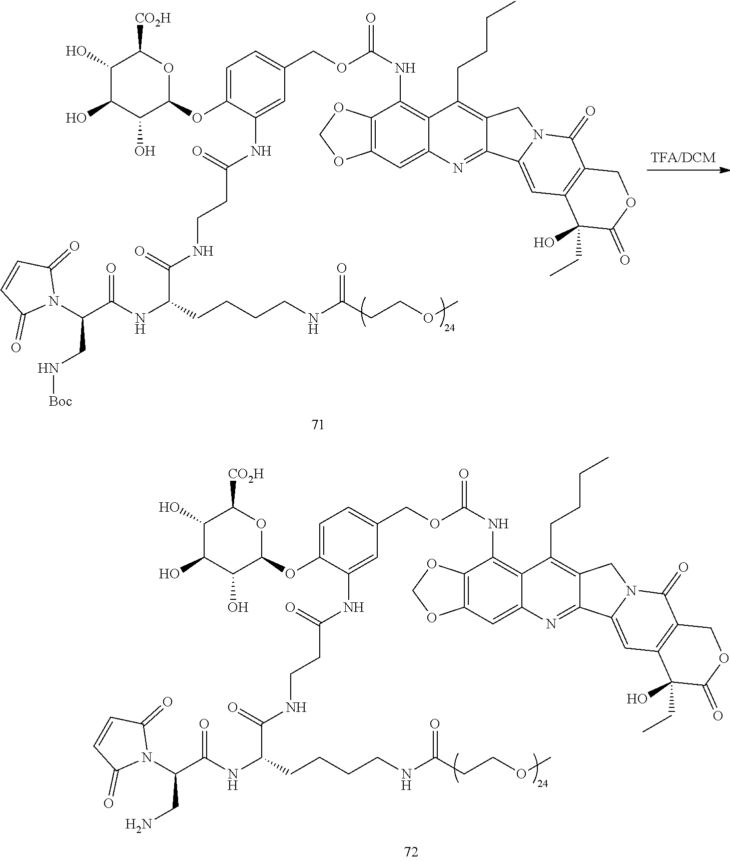

Compound 71 was solubilized in anhydrous DCM (0.45 mL) and cooled to 0° C. TFA (0.05 mL) was added and the reaction was stirred for 3 h. The reaction was then diluted with DMSO, DCM was removed in vacuo, then purified by preparative HPLC to provide compound 72 (10 mg, 88%). LC-MS (Method A): t_{R}=1.32 min; MS m/z (ES+) found 1135.85 (½ mass).

### Synthetic Example 48

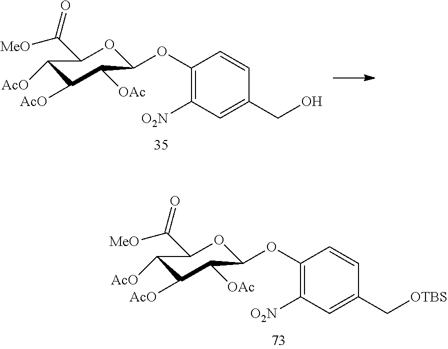

Compound 35 (660 mg, 1.36 mmol), prepared according to the procedure in Bioconjugate Chem. (2006) 17: 831-840, was dissolved in DCM (5 mL). DIPEA (0.71 mL, 4.1 mmol) was added followed by slow addition of TBSOTf (0.34 mL, 1.5 mmol). The reaction was stirred for 5 minutes, quenched with MeOH and concentrated in vacuo. The crude product was purified by column chromatography 25 G KP-Sil 10-80% EtOAc in Hex. Fractions containing the desired product were concentrated in vacuo to afford compound 73 as a colorless solid (731 mg, 1.22 mmol, 90%). LC-MS (Method A): t_{R}=2.44 min; MS (m/z) [M+Na]⁺ calc. for C₂₆H₃₇NNaO₁₃Si 622.19, found 622.10.

### Synthetic Example 49

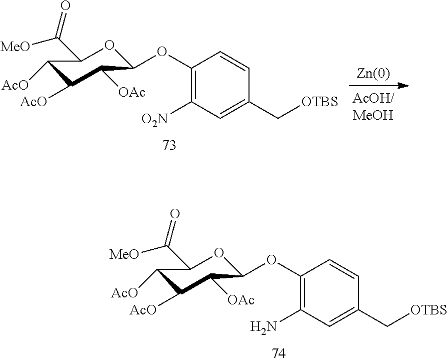

Compound 73 (731 mg, 1.22 mmol) was dissolved in 5:1 MeOH:AcOH (10 mL). Zinc dust (2.39 g, 36.6 mmol) was added to reaction. The reaction was stirred for 10 minutes them filter through a bed of Celite and rinsed with MeOH. The eluent was concentrated and purified by column chromatography 25 G KP-Sil 10-100% EtOAc in Hex. Fractions containing the desired product were concentrated to afford compound 74 as a colorless solid (693 mg, 1.22 mmol, 99%). LC-MS (Method A): t_{R}=2.40 min; MS (m/z) [M+H]⁺ calc. for C₂₆H₄₀NO₁₁Si 570.24, found 571.08.

### Synthetic Example 50

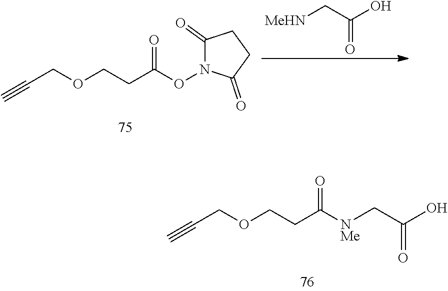 [SMILES: C#CCOCCC(=O)ON1C(=O)CCC1=O ; CNCC(=O)O ; C#CCOCCC(=O)N(C)CC(=O)O]

Compound 75 (1.05 g, 4.66 mmol), referred to as PropargOPr and obtained from Click Chemistry Tools (CAS:1174157-65-3), was dissolved in DMF (10 mL). H-Sar-OH (831 mg, 9.33 mmol), which is N-methyl glycine, and DIPEA (2.4 mL, 14 mmol) were added. The reaction was stirred for 45 minutes, quenched AcOH, and purified by prep-HPLC. Fractions containing the desired product were concentrated to afford compound 76 as a colorless solid (821.3 mg, 4.12 mmol, 88%). LC-MS (Method A): t_{R}=0.81 min; MS (m/z) [M+H]⁺ calc. for C₉H₁₄NO₄ 200.09, found 199.72.

### Synthetic Example 51

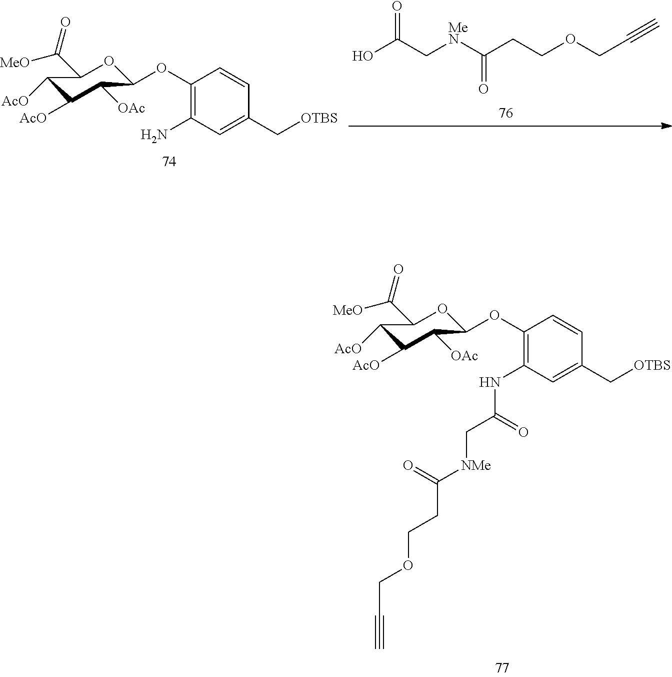

Compound 74 (636 mg, 1.22 mmol) from Example 49 was dissolved in DMF (5 mL). DIPEA (1.06 mL, 6.08 mmol), compound 76 (727 mg, 3.65 mmol) from Example 50 and HATU (1.38 g, 3.65 mmol) were added to the reaction. The reaction was stirred for 90 minutes then diluted in EtOAc (200 mL), washed with sat NaHCO₃ (200 mL), and H₂O (2×200 mL). The organic portion was dried with MgSO₄, filtered, and concentrated in vacuo. The crude product was purified by column chromatography 10-80% EtOAc in Hex. Fractions containing the desired product were concentrated to afford compound 77 as a colorless solid (823 mg, 1.10 mmol, 90%). LC-MS (Method A): t_{R}=2.34 min; MS (m/z) [M+H]⁺ calc. for C₃₅H₅₁N₂O₁₄Si 751.31, found 751.22.

### Synthetic Example 52

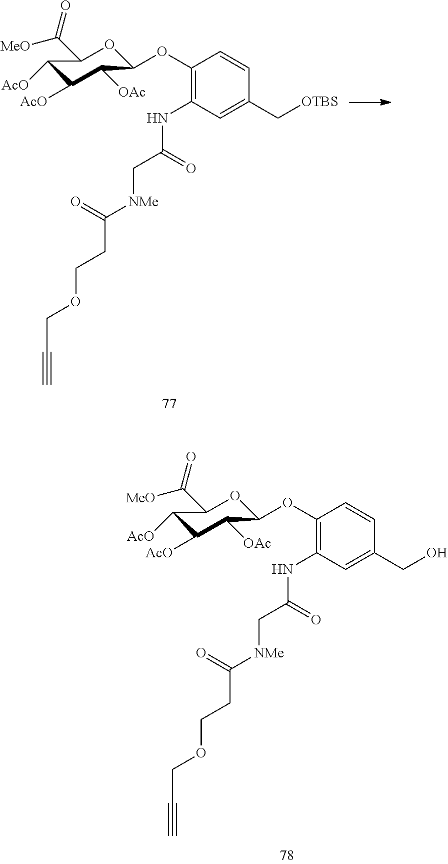

Compound 77 (823 mg, 1.10 mmol) was dissolved in 1:1:1 THF:H₂O:AcOH and stirred at room temperature for 24 hours. The reaction was concentrated in vacuo, diluted EtOAc (200 mL), washed with sat. NaHCO₃(3×200 mL), dried MgSO₄, filtered and concentrated in vacuo to afford compound 78 as a colorless solid (601 mg, 0.944 mmol, 86%). LC-MS (Method A): t_{R}=1.55 min; MS (m/z) [M+H]⁺ calc. for C₂₉H₃₇N₂O₁₄ 637.22, found 637.04.

### Synthetic Example 53

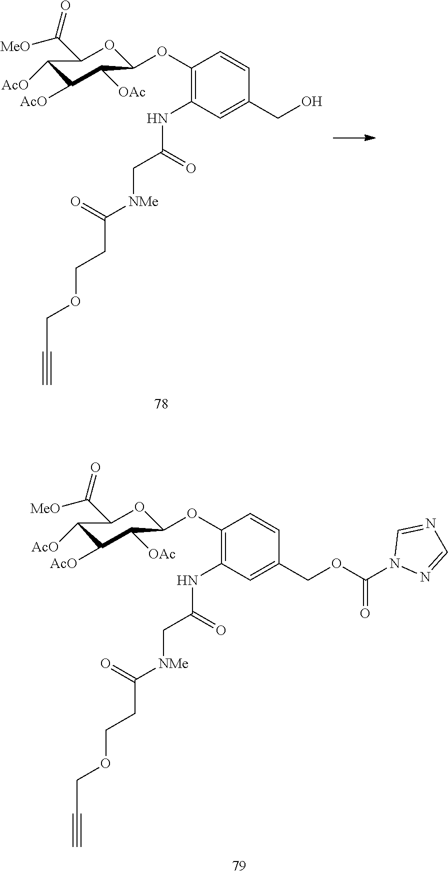

Compound 78 (300 mg, 0.47 mmol) was dissolved in DCM (2 mL). 1,1′-Carbonyl-di-(1,2,4-triazole) (232 mg, 1.41 mmol) was added. The reaction was stirred for 30 minutes then diluted into EtOAc (50 mL), washed with H₂O (3×50 mL), dried MgSO₄, filtered and concentrated in vacuo to afford compound 79 as a colorless solid (340 mg, 0.465 mmol, 99%), which was used in subsequent steps without purification. LC-MS (Method A): t_{R}=1.68 min; MS (m/z) [M+H]⁺ calc. for C₃₂H₃₈N₅O₁₅ 732.24, found 732.11.

### Synthetic Example 54

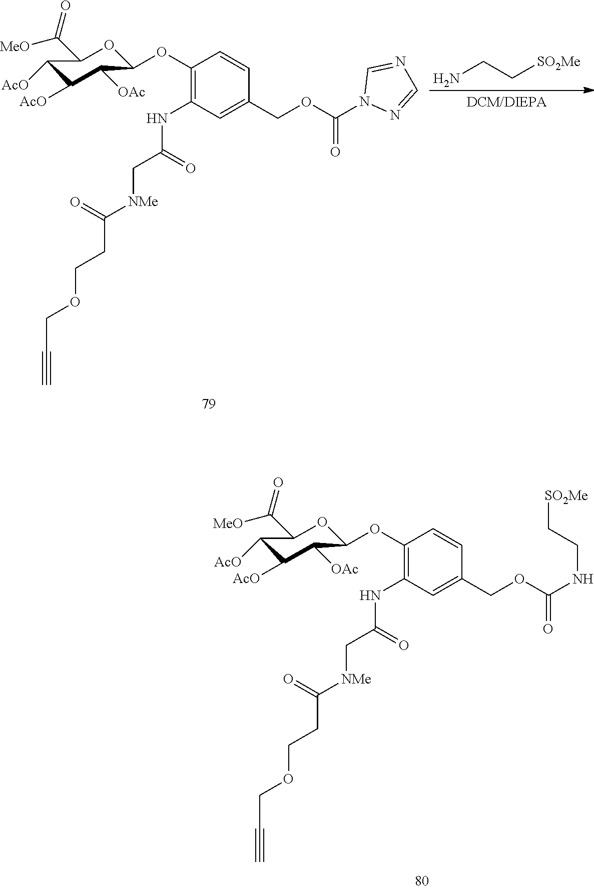

Compound 79 (200 mg, 0.273 mmol) was dissolved in DCM (2 mL). 2-(methylsulfonyl)-ethanamine (54 μL, 0.547 mmol) purchased from Enamine and DIPEA (0.14 mL, 0.82 mmol) were added to the reaction. The reaction was stirred for 10 minutes then diluted into EtOAc (50 mL), washed with 1M HCl (3×50 mL), H₂O (50 mL), dried MgSO₄, filtered, and concentrated in vacuo to afford compound 80 as a colorless solid (210 mg, 0.267 mmol, 98%). LC-MS (Method A); t_{R}=1.64 min; MS (m/z) [M+H]⁺ calc. for C₃₃H₄₄N₃O₁₇S 786.24, found 786.14.

### Synthetic Example 55

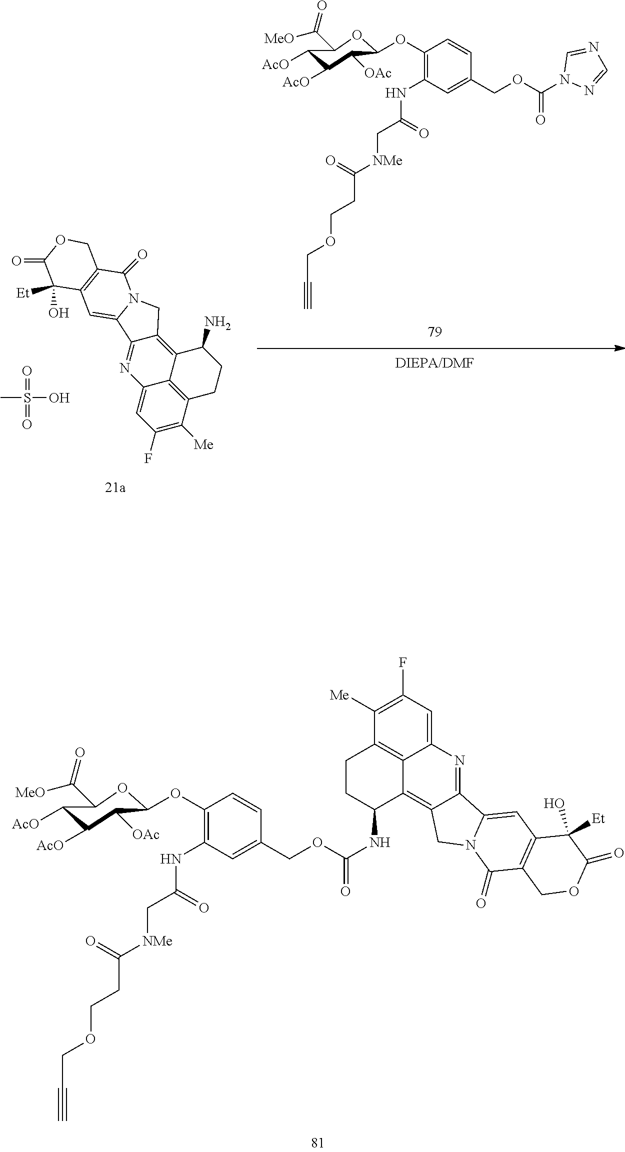

Exatecan mesylate (21a, 10.0 mg, 0.0188 mmol) was dissolved in anhydrous DMF (0.5 mL). DIPEA (16 μL, 0.094 mmol) and compound 79 (41.3 mg, 0.0.564 mmol) were added to the reaction. The reaction was heated at 60° C. for 5 h. The reaction was quenched with AcOH and purified by prep-HPLC. Fractions containing the desired product were lyophilized to afford compound 81 as a yellow powder (1.4 mg, 1.3 μmol, 6.8%). LC-MS (Method A): t_{R}=2.10 min; MS (m/z) [M+H]⁺ calc. for C₅₄H₅₇N₅FO₁₉ 1098.36, found 1098.51.

### Synthetic Example 56

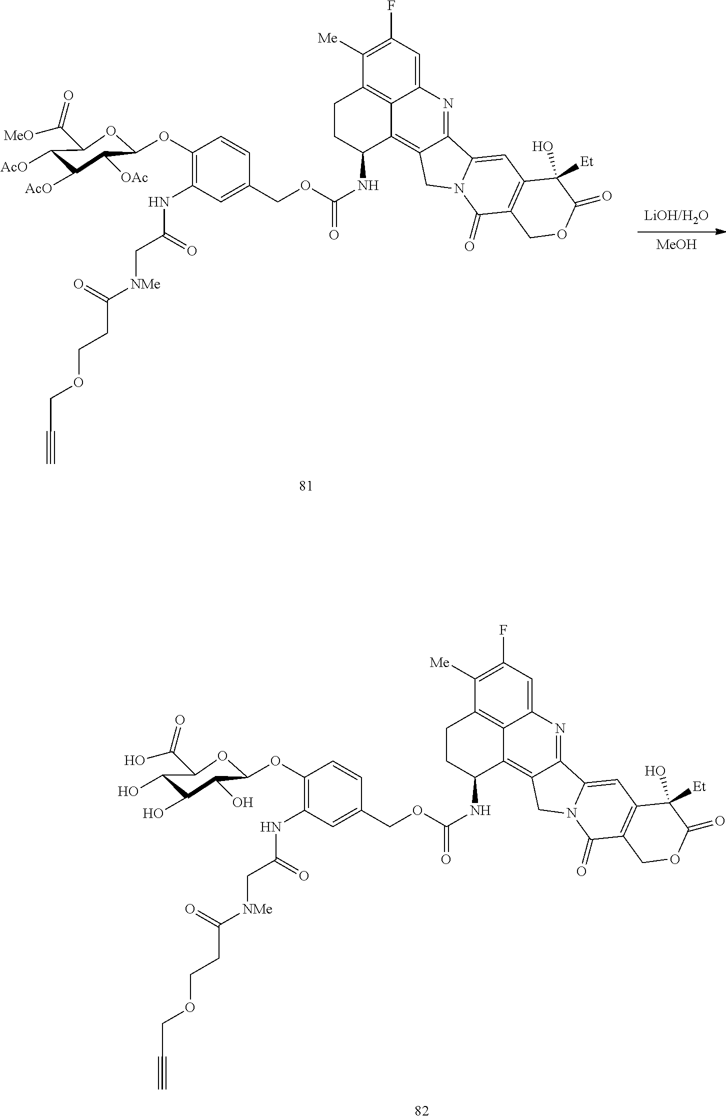

Compound 81 (1.4 mg, 1.3 μmol) was dissolved in MeOH (0.5 mL). LiOH (5 mg, 0.209 mmol) was added and the reaction was sonicated to aid dissolution and stirred for 5 mins. H₂O (0.5 mL) was added to the reaction and stirred for 5 minutes, then quenched with AcOH and concentrated in vacuo. The reaction was purified by prep-HPLC 10 mm 5-95% MeCN in H₂O. Fractions containing the desired product were lyophilized to afford compound 82 as a yellow powder (0.7 mg, 0.7 μmol, 57%). LC-MS (Method A): t_{R}=1.62 min. MS (m/z) [M+H]⁺ calc. for C₄₇H₄₉FN₅O₁₆ 958.32, found 958.62.

### Synthetic Example 57

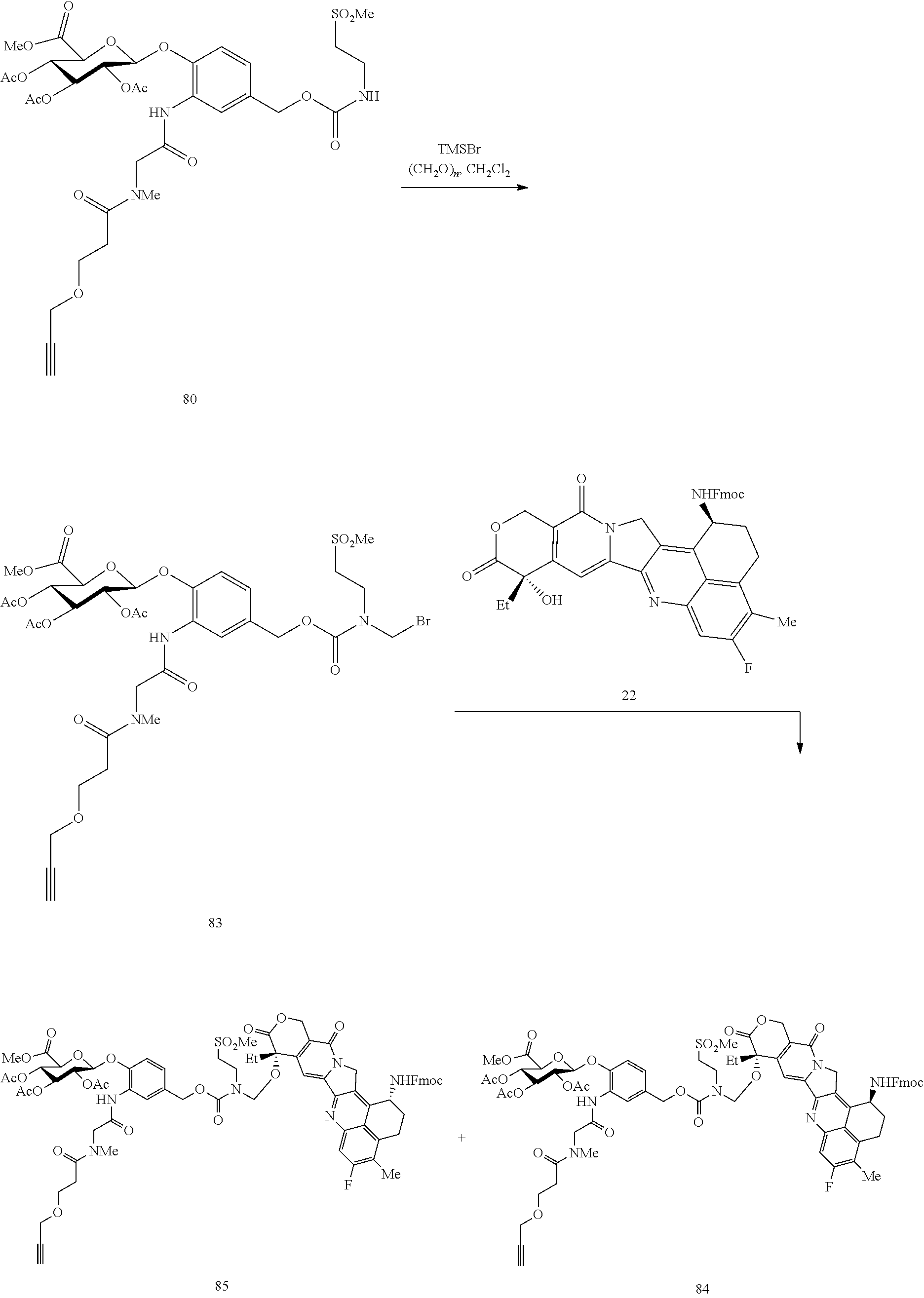

Compound 80 (50.0 mg, 0.0636 mmol) was dissolved in DCM (1 mL). Paraformaldehyde (100 mg, 3.3 mmol) was added to the reaction followed by TMSBr (21 μL, 0.16 mmol). The reaction was stirred for 15 minutes. An aliquot was quenched in MeOH and complete conversion to the MeOH adduct was observed by UPLC-MS. The reaction was filtered through a 0.45 μm PTFE filter, rinsed DCM (2×2 mL), and toluene (2 mL) was added to azeotrope the final mixture. The eluent was concentrated to afford compound 83 as a colorless solid which was used immediately in the next step.

Compound 83 (0.0636 mmol) was dissolved in anhydrous DCM (0.5 mL). 1,2,2,6,6-Pentamethylpiperidine (21 μL, 0.11 mmol) was added to the reaction, and the reaction solution was added directly to the compound 22 solid (12.0 mg, 0.0183 mmol). The reaction was stirred at room temperature for 3.5 h at which time complete conversion was observed by UPLC-MS. The reaction was quenched with AcOH, concentrated and purified by prep-HPLC 21 mm 10-95% MeCN in H₂O. Fractions containing the desired product were concentrated to afford compound 84 as a yellow solid (5.4 mg, 3.7 μmol, 20%). LC-MS (Method A): t_{R}=2.30 min; MS (m/z) [M+H]⁺ calc. for C₇₃H₇₆FN₆O₂₃S 1455.47, found 1455.43. Fractions containing an observed product presumed to be epimerization of the exatecan FMOC protected amine were concentrated in vacuo to afford compound 85 as a yellow solid (2.1 mg, 1.4 μmol, 8%). LC-MS (Method A): t_{R}=2.33 min; MS (m/z) [M+H]⁺ calc. for C₇₃H₇₆FN₆O₂₃S 1455.47, found 1455.63.

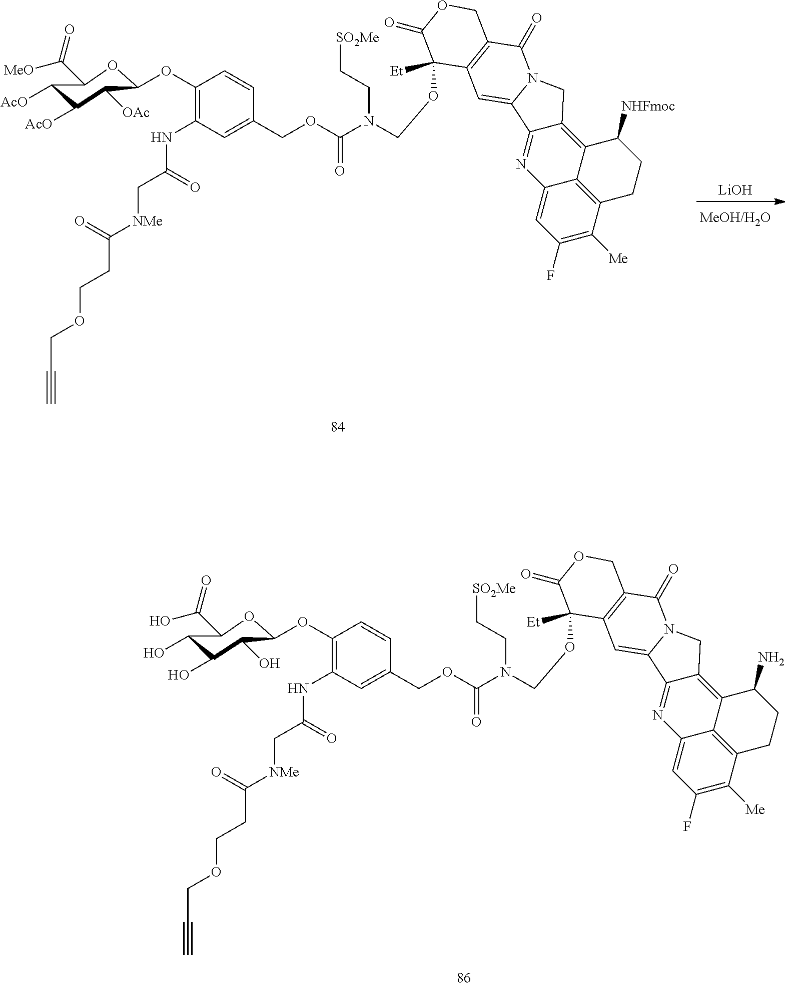

Compound 84 (5.4 mg, 3.7 μmol) was dissolved in MeOH (1 ml). LiOH (25 mg) was added and the reaction was sonicated to aid in dissolution. The reaction was stirred for 10 minutes and H₂O (1 mL) was added and stirred for an additional 20 minutes. The reaction was quenched with AcOH, concentrated, and purified by prep-HPLC 10 mm 5-60-95% MeCN in H₂O 0.05% TFA. Fractions containing the desired product were lyophilized to afford compound 86 as a yellow powder (1.96 mg, 1.79 μmol, 48%). LC-MS (Method A): t_{R}=1.22 min; MS (m/z) [M+H]⁺ calc. for C₅₁H₅₈FN₆O₁₈S 1093.35, found 1093.56.

### Synthetic Example 58

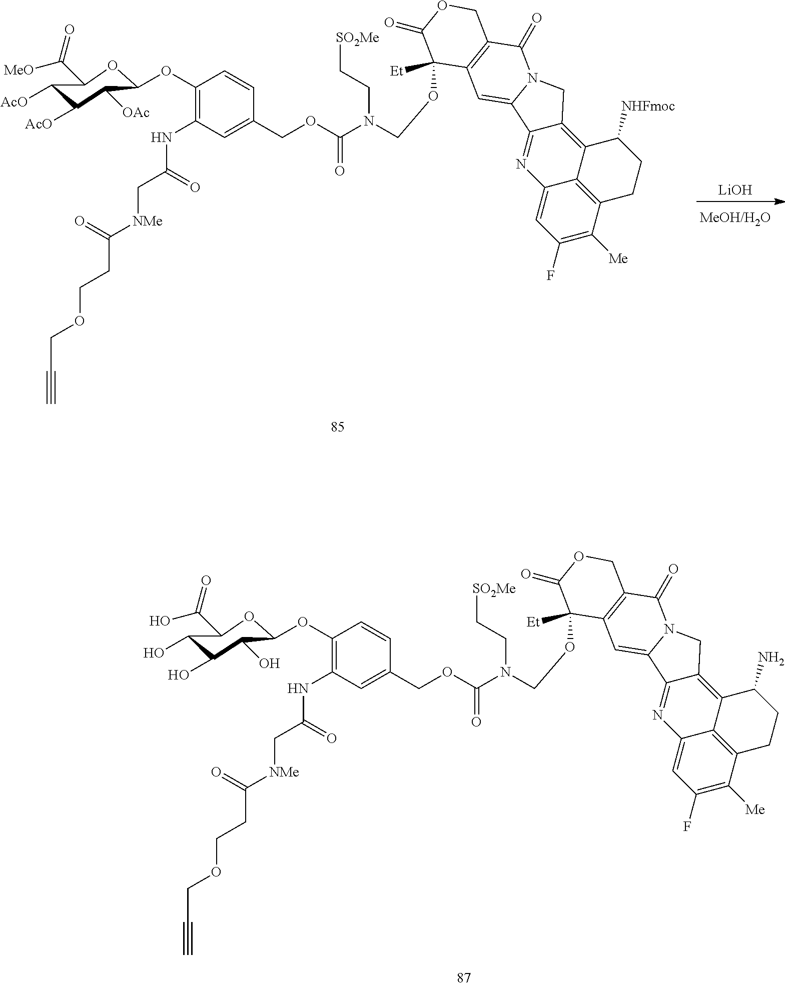

Compound 85 from Example 57 (2.1 mg, 1.4 μmol) was dissolved in MeOH (1 mL). LiOH (25 mg) was added and the reaction was sonicated to aid in dissolution. The reaction was stirred for 10 minutes and H₂O (1 mL) was added and stirred for an additional 20 minutes. The reaction was quenched with AcOH, concentrated, and purified by prep-HPLC 10 mm 5-60-95% MeCN in H₂O 0.05% TFA. Fractions containing the desired product were lyophilized to afford compound 87 as a yellow powder (0.98 mg, 0.90 μmol, 62%). LC-MS (Method A): t_{R}=1.40 min; MS (m/z) [M+H]⁺ calc. for C₅₁H₅₈FN₆O₁₈S 1093.35, found 1093.18.

### Synthetic Example 59

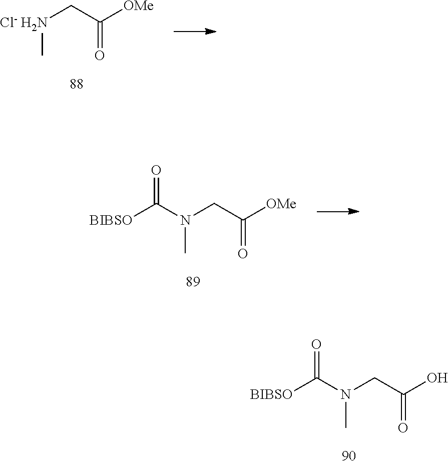

Sarcosine methyl ester HCl (88, 5.00 g, 35.8 mmol) was suspended in anhydrous DCM (100 mL). DIPEA (18.7 mL, 107.5 mmol) was added and the reaction was sonicated and stirred vigorously to solubilize H-Sar-OMe. The solution was slightly opaque. CO₂ was bubbled through the reaction for 30 minutes. Di-t-butylisobutylsilyl trifluoromethanesulfonate (BIBSOTf, 20 mL, 72 mmol) purchased from Gelest, Inc. was added to the reaction and stirred for 1 hour. A pellet of dry ice was added to the reaction mixture. After cessation of bubbling, the reaction was concentrated, diluted with Hex (200 mL), washed 1M aq. HCl (3×200 mL), dried MgSO₄, filtered and concentrated in vacuo. The crude product was purified by column chromatography 0-10% EtOAc in Hex. R_{f}=0.25 in 9:1 Hex:EtOAc stain KMnO₄. Fractions containing the desired product were concentrated in vacuo to afford compound 89 as a colorless oil (10.38 mg, 30.03 mmol, 84%). LC-MS (Method C): t_{R}=1.67 min; MS (m/z) [M+H]⁺ calc. for C₁₇H₃₆NO₄Si 346.24, found 346.99.

### Synthetic Example 60

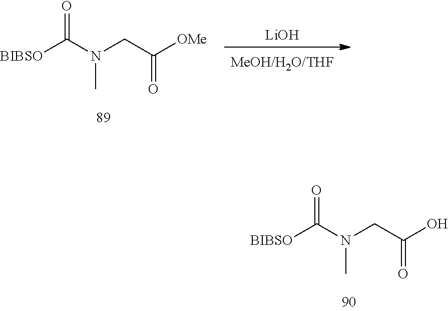

Compound 89 was dissolved in 1:1:1 THF:MeOH:H₂O (60 mL). LiOH (1.80 g, 75.1 mmol) was added and the reaction was stirred for 10 minutes at which point complete conversion was observed by UPLC-MS. The reaction was quenched with AcOH, concentrated in vacuo and purified by column chromatography 0-10% MeOH in DCM. Fractions containing the desired product were concentrated in vacuo to afford compound 90 as a colorless solid (8.79 mg, 26.5 mmol, 88%). LC-MS (Method C): t_{R}=1.50 min; MS (m/z) [M+H]⁺ calc. for C₁₆H₃₄NO₄Si 332.23, found 331.86.

### Synthetic Example 61

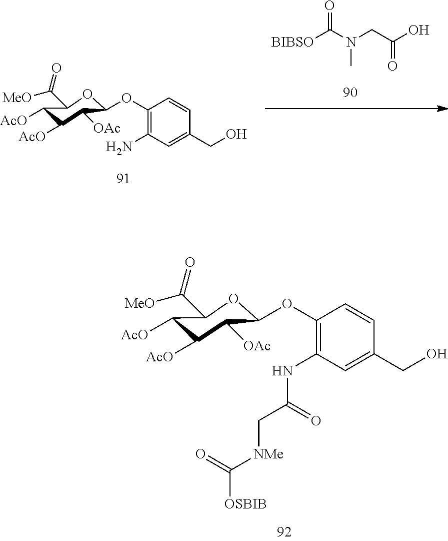

Compound 91 (4.00 g, 8.78 mmol), prepared according to the method of Bioconjugate Chem. (2006) 17: 831-840, was dissolved in DCM (10 mL). BIBS-Sar-OH (90, 5.82 g, 17.6 mmol) from Example 60 was added followed by EEDQ (6.52 g, 26.4 mmol). The reaction was stirred for 90 minutes. The reaction was diluted with EtOAc (200 mL), washed with 1M aq. HCL (3×200 mL), sat. NaHCO₃ (3×200 mL), water (200 mL), dried MgSO₄, filtered and concentrated in vacuo. The crude product was purified by column chromatography 0-60% EtOAc in Hex. Fractions containing the desired product were concentrated in vacuo to afford compound 92 as a colorless solid (5.16, 6.71 mmol, 76%). LC-MS: t_{R}=1.58 min; MS (m/z) [M+H]⁺ calc. for C₃₆H₅₇N₂O₁₄Si 769.36, found 769.29.

### Synthetic Example 62

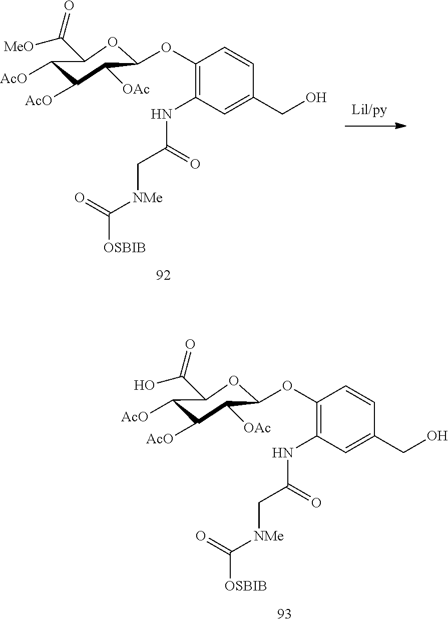

Compound 92 (2.70 g, 3.51 mmol) was dissolved in anhydrous pyridine (10 mL). LiI (2.82 g, 21.1 mmol) was added and the reaction was sealed and heated at 115° C. overnight (˜16 h). The reaction was diluted with EtOAc (200 mL), washed 1M aq. HCl (3×200 mL), washed H₂O, dried MgSO₄, filtered and concentrated in vacuo. The crude product was purified by column chromatography 0-60% EtOAc in Hex. Fractions containing the desired product were concentrated in vacuo to afford compound 93 as a colorless solid (2.06 g, 2.73 mmol, 78%). LC-MS (Method C): t_{R}=1.50 min; MS (m/z) [M+H]⁺ calc. for C₃₅H₅₅N₂O₁₄Si 755.34, found 755.32.

### Synthetic Example 63

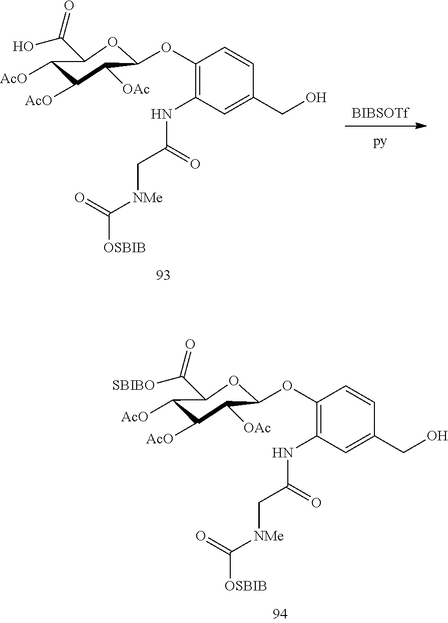

Compound 93 (700 mg, 0.927 mmol) was dissolved in pyridine (2 mL). BIBSOTf (0.78 mL, 2.78 mmol) purchased from Gelest Inc. was added. The reaction was stirred for 30 minutes, diluted with EtOAc (50 mL), washed with 1M HCl (3×50 mL), dried MgSO₄, filtered and concentrated in vacuo. The crude product was purified by column chromatography 0-60% EtOAc in Hex. Fractions containing the desired product were concentrated in vacuo to afford compound 94 as a colorless solid (723 mg, 0.758 mmol, 82%). LC-MS (Method C): t_{R}=1.85 min; MS (m/z) [M+H]⁺ calc. for C₄₇H₈₁N₂O₁₄Si₂ 953.52, found 953.34.

### Synthetic Example 64

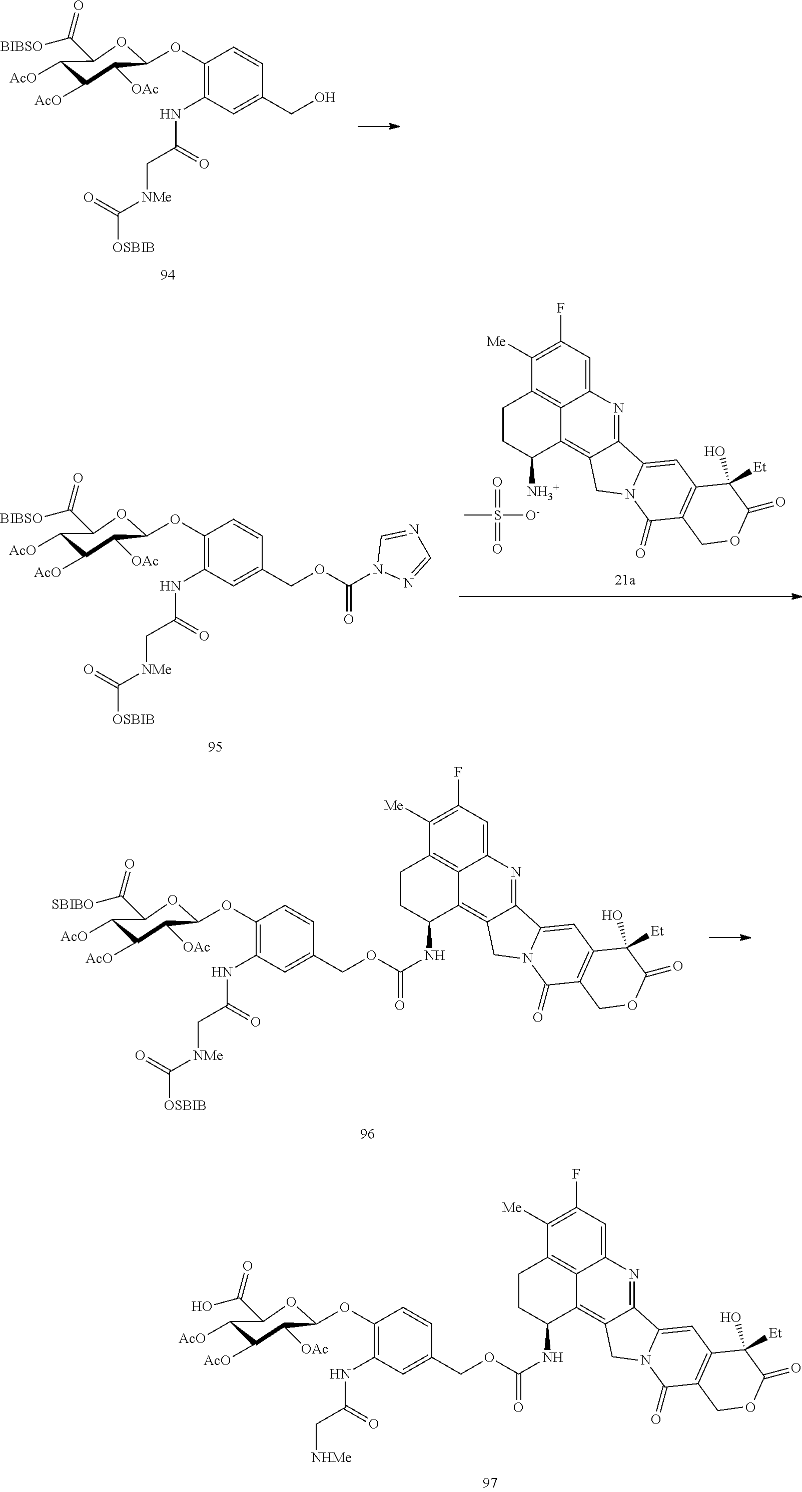

Compound 94 (723 mg, 0.758 mmol) was dissolved in DCM (2 mL). CDT (373 mg, 2.28 mmol) was added and the reaction was stirred for 30 minutes. The reaction was diluted with EtOAc (50 mL), washed with H₂O (3×50 mL), dried MgSO₄, filtered and concentrated in vacuo to afford compound 95 as a colorless solid (790 mg, 0.753 mmol, 99%), which was used in next step without further purification. LC-MS (Method C): t_{R}=1.86 min; MS (m/z) [M+H]⁺ calc. for C₅₀H₈₂N₅O₁₅Si₂ 1048.53, found 1049.29

Compound 95 (395 mg, 0.376 mmol) was dissolved in 0.5 mL anhydrous DMF and added directly to exatecan mesylate (21-a, 25 mg, 0.047 mmol) solid followed by DIPEA (0.081 mL, 0.47 mmol). The reaction was stirred overnight (approximately 15 h). Complete conversion was observed by UPLC-MS. The reaction was diluted with EtOAc (20 mL), washed with sat. NH₄Cl (3×20 mL), dried MgSO₄, filtered and concentrated in vacuo to afford compound 96 as a crude product, which was used in the next step without further purification. LC-MS (Method C): t_{R}=1.89 min; MS (m/z) [M+H]⁺ calc. for C₇₂H₁₀₁N₅O₁₉Si₂ 1414.66, found 1414.71.

Crude compound 96 (0.047 mmol) was dissolved in 1 mL anhydrous DMF. AcOH (200 μL) was added. TBAF 1M in THE (0.28 mL) was added to the reaction. Complete conversion was observed after 45 min. Silica (100 mg) was added to quench fluoride. The reaction was filtered and purified by Prep-HPLC 21 mm 10-95% MeCN in H₂O 0.05% TFA. Fractions containing the desired product were lyophilized to afford compound 97 as a yellow powder (45 mg, 0.046 mmol, 98%). LC-MS (Method A): t_{R}=1.53 min; MS (m/z) [M+H]⁺ calc. for C₄₇H₄₉N₅O₁₇ 974.31, found 974.10.

### Synthetic Example 65

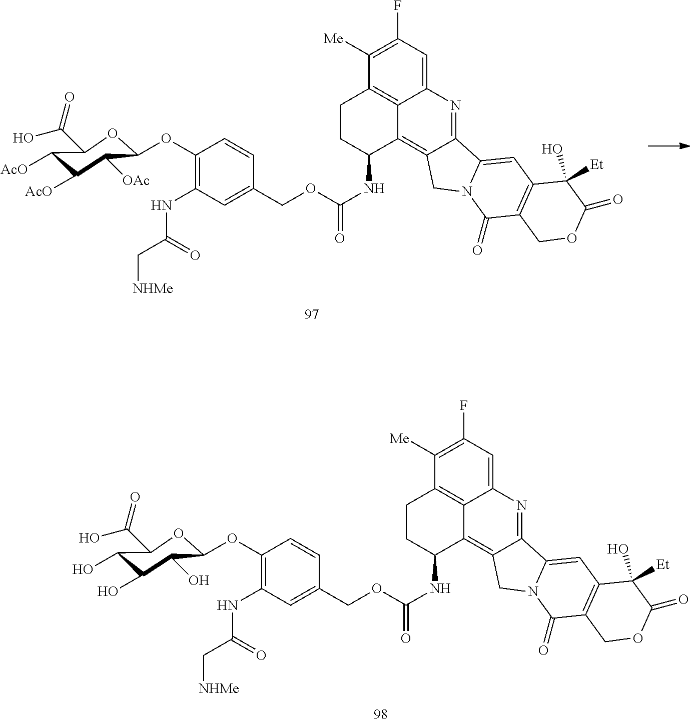

Compound 97 (45 mg, 0.046 mmol) was diluted to form a 10 mM DMSO solution (4.6 mL). PBS 7.4 (10×, 4.6 mL) was added to make a 5 mM solution. A solution of acetyl esterase 800 Units/mL added to form a 2.5 mM drug linker solution (9.2 mL). The reaction was stirred at 40° C. overnight (approximately 15 h). Complete conversion was observed. The reaction was diluted into 200 mL cold MeOH, centrifuged, supernatant collected, concentrated, and purified by Prep-HPLC 21 mm 10-95% MeCN in H₂O 0.05% TFA. Fractions containing the desired product lyophilized to afford compound 98 as a yellow powder (30 mg, 0.035 mmol). LC-MS (Method A): t_{R}=1.30 min; MS (m/z) [M+H]⁺ calc. for C₄₁H₄₃N₅O₁₄ 848.28, found 847.97.

### Synthetic Example 66

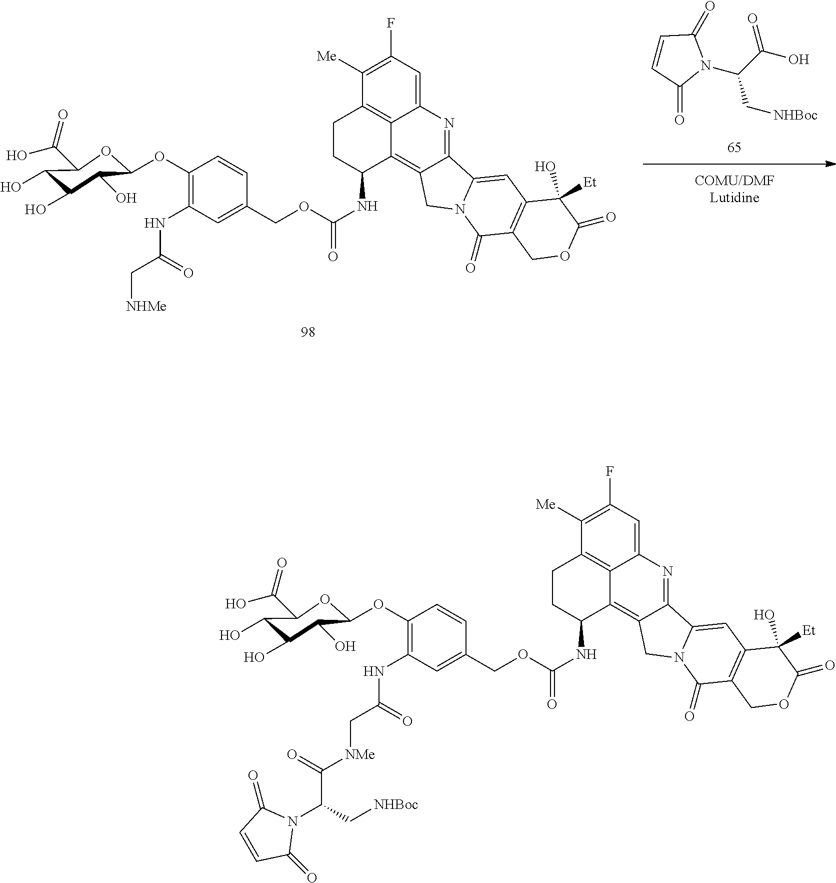

Maleimido-Dpr(Boc)-OH (65, 26.6 mg, 0.0936 mmol), prepared by the procedure of Nature Biotechnology (2014) 32: 1059-1062, was dissolved in 0.5 mL anhydrous DMF cooled to 0° C. Lutidine (0.022 mL, 0.19 mmol) was added followed by COMU (38.7 mg, 0.0905 mmol). The reaction was stirred for 30 minutes. The activated Maleimido-DPr(Boc)-OH solution was added directly to compound 98 (30 mg, 0.035 mmol) solid. The reaction was stirred for 60 minutes at which point complete conversion was observed. Reaction was quench with AcOH and purified by Prep-HPLC 21 mm 10-95% MeCN in H₂O. Fractions containing the desired product was lyophilized to afford compound 99 as a yellow solid (4.5 mg, 4.0 μmol, 13%). LC-MS (Method A): t_{R}=1.72 min; MS (m/z) [M+H]⁺ calc. for C₅₃H₅₇N₇O₁₉ 1114.37, found 1114.69.

### Synthetic Example 67

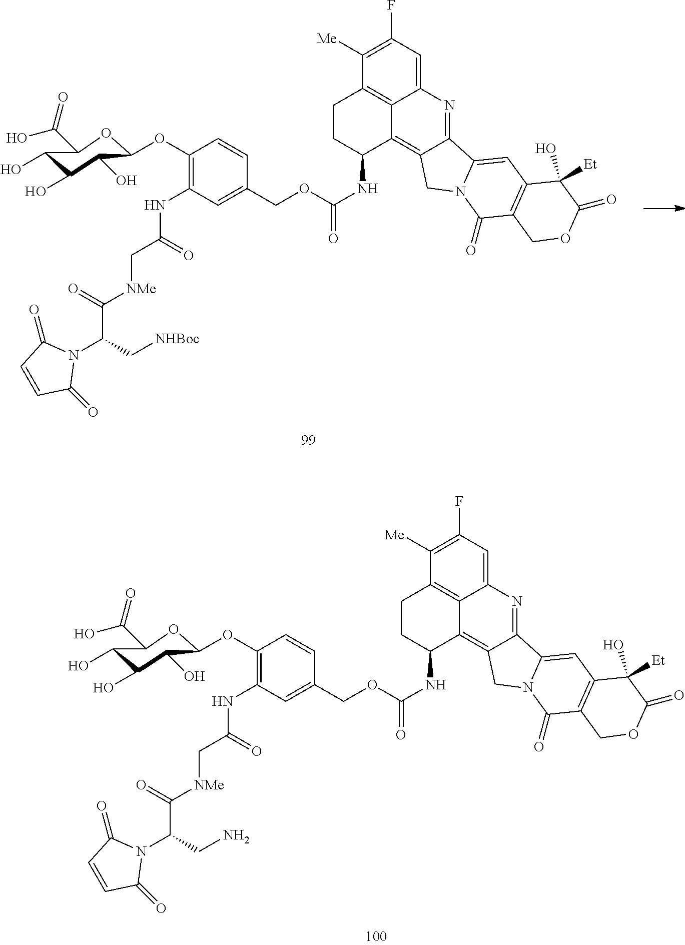

Compound 99 (4.5 mg, 4.0 μmol) was dissolved in 20% TFA in DCM. Complete conversion to compound 100 was observed by UPLC-MS after 25 minutes. The reaction was concentrated in vacuo and purified by prep-HPLC 10 mm 10-95% MeCN in H₂O 0.05% TFA. Fractions containing the desired product were lyophilized to afford compound 100 as a white powder (3.91 mg, 3.86 μmol, 96%). LC-MS (Method A): t_{R}=1.31 min; MS (m/z) [M+H]⁺ calc. for C₄₈H₅₀N₇O₁₇ 1014.32, found 1014.07.

### Synthetic Example 68

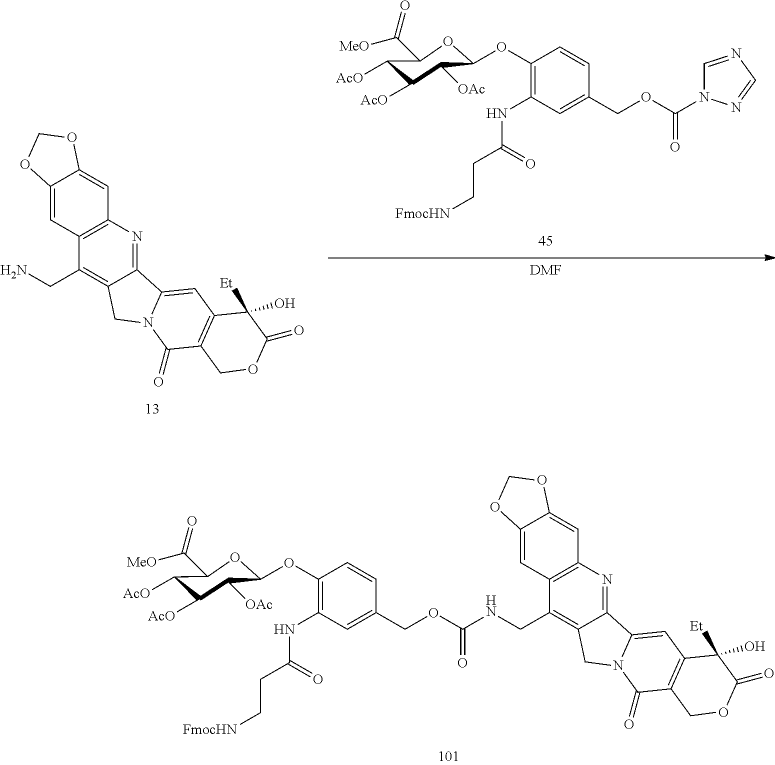

Compound 45 (82 mg, 0.097 mmol), prepared according to the procedure of Example 26, and compound 13 (14 mg, 0.033 mmol), referred to as 7-MAD-MDCPT, were dissolved in DCM (2 mL) followed by DMF (0.5 mL). DCM was evaporated off to concentrate the reaction mixture into DMF. No conversion to product observed after 30 minutes, additionally no hydrolysis of compound 45 was observed. DIPEA (0.1 mL) was added to the reaction mixture whereupon the clear red reaction mixture became opaque. Reaction allowed to stir overnight (˜15 h) at room temperature. Approximately 50% conversion of drug to the desired drug linker product was observed, ˜10% hydrolysis of activated CDT linker remaining. [Note: Using a MeOH diluent for UPLC-MS analysis the Linker-CDT was observed at t=30 min (reaction neutral), Linker-OCOMe observed at t=15 h (after base addition)]. Reaction was concentrated slowly at 45° C. under vacuum for 60 minutes. Reaction mixture heated at 60° C. for 4 hours until complete conversion was observed by UPLC-MS. Reaction was concentrated in vacuo, then purified by column chromatography 0-5% MeOH in DCM. Fractions containing the desired product were concentrated to afford compound 101 as a white solid (28 mg, 0.023 mmol, 70%). LC-MS (Method A): t_{R}=2.17 min; MS (m/z) [M+H]⁺ calc. for C₆₁H₅₈N₅O₂₁ 1196.36, found 1196.19.

### Synthetic Example 69

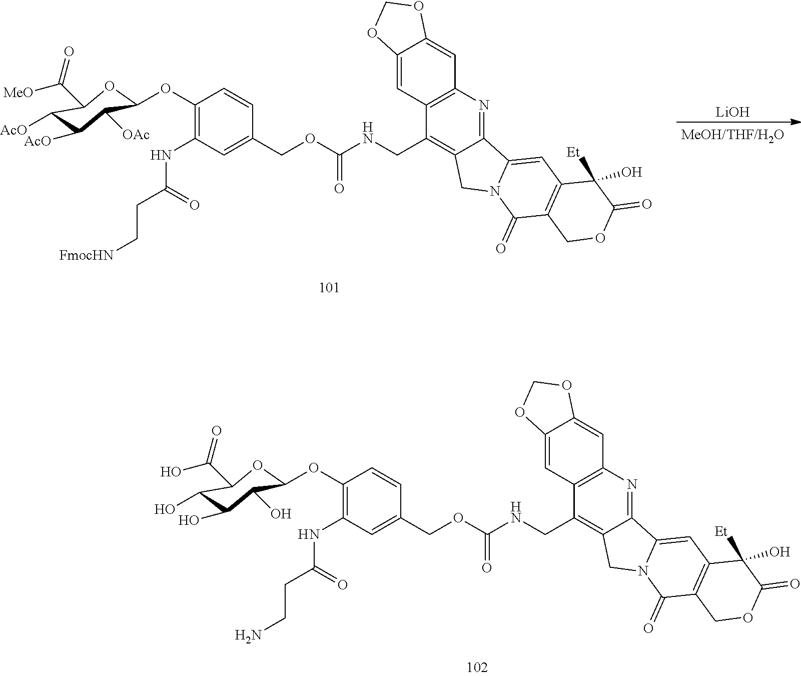

Compound 101, 28 mg, 0.023 mmol) was dissolved in MeOH (0.5 mL) and THF (0.5 mL). LiOH (25 mg, 1.0 mmol) was added. The reaction was sonicated to solubilize LiOH and then stirred. After 10 minutes water was added (0.5 mL). Complete conversion to the deprotected glucuronide was observed by UPLC-MS after 90 minutes. Piperidine (0.05 mL) was added. Complete deprotection of the Fmoc was observed after 60 additional minutes. The reaction was quenched with AcOH (0.2 mL). The reaction was concentrated, then purified by Prep-HPLC using a 10 mm Max-RP with a 5-60-95 MeCN in H₂O 0.05% TFA gradient. Fractions containing the desired product were concentrated in vacuo to afford compound 102 as a yellow solid (10.1 mg, 0.0121 mmol, 51.8%). LC-MS (Method A): t_{R}=1.05 min; MS (m/z) [M+H]⁺ calc. for C₃₉H₄₀N₅O₁₆ 834.25, found 833.71.

### Synthetic Example 70

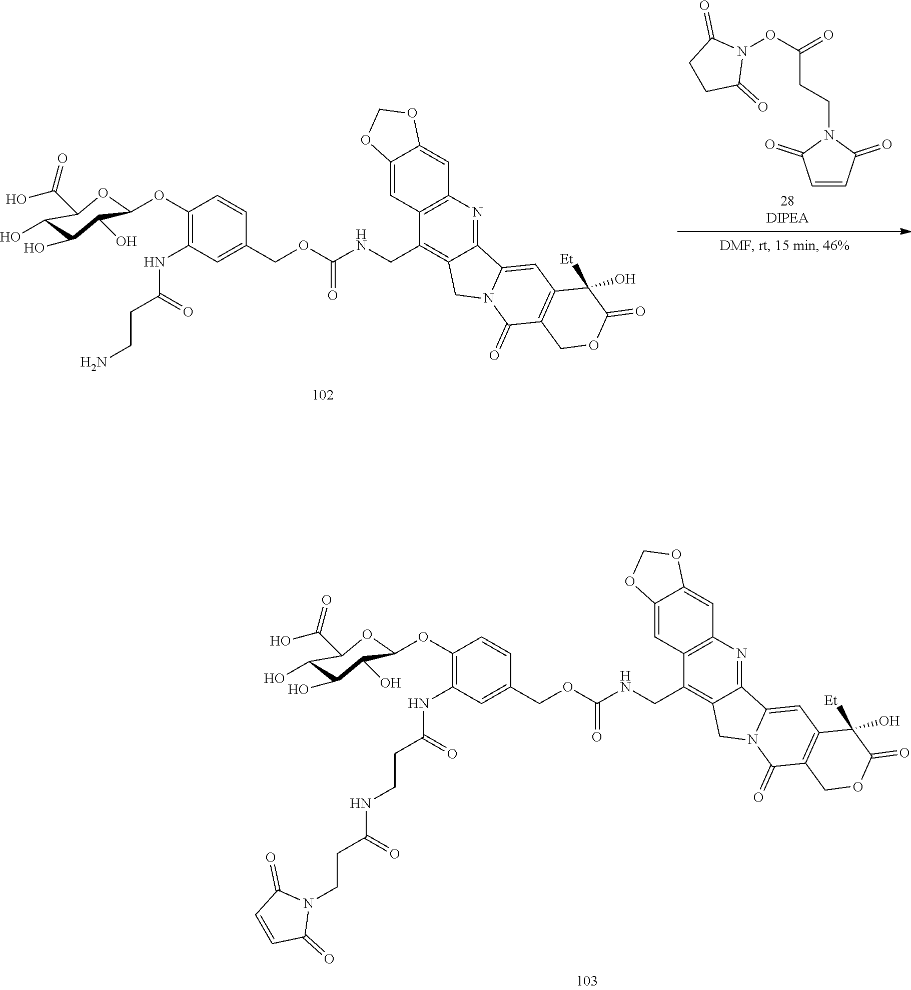

Compound 102 (10.1 mg, 0.0121 mmol) was dissolved in DMF (0.2 mL). 3-(Maleimido)propionic acid N-hydroxysuccinimide ester (9.7 mg, 0.036 mmol) was added. DIPEA (13 μL, 0.073 mmol) was added. The reaction was stirred for 15 minutes at which point complete conversion was observed by UPLC-MS. The reaction was quenched with AcOH (0.05 mL), then purified by Prep-HPLC 10 mm Max RP C12 5-60-95% MeCN in H₂O 0.05% TFA. Fractions containing the desired product were lyophilized to afford compound 103 as a yellow powder (5.53 mg, 0.00561 mmol, 46.4%). LC-MS (Method A) t_{R}=1.24 min; MS (m/z) [M+H]⁺ calc. for C₄₆H₄₅N₆O₁₉ 985.27, found 985.45.

### Synthetic Example 71

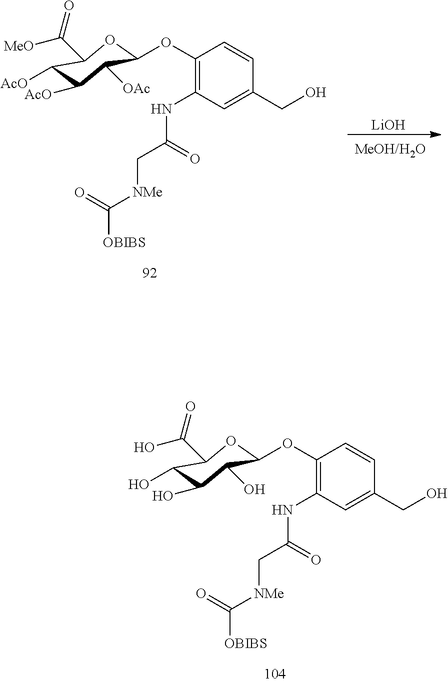

Compound 92 (500 mg, 0.65 mmol), prepared according to the procedure of Example 60, was dissolved in MeOH (10 mL). LiOH (500 mg, 21 mmol) added. The reaction was sonicated, and stirred for 5 min. H₂O added, stir 5 min. Complete conversion observed. The reaction was quench with AcOH, concentrated in vacuo and purified by Prep-HPLC. Fractions containing desired product were concentrated in vacuo to afford compound 104 as a colorless solid (295 mg, 0.469 mmol, 72%). LC-MS (Method C): t_{R}=1.23 min; MS (m/z) [M+H]⁺ calc. for C₂₉H₄₉N₂O₁₁Si 629.31, found 629.01.

### Synthetic Example 72

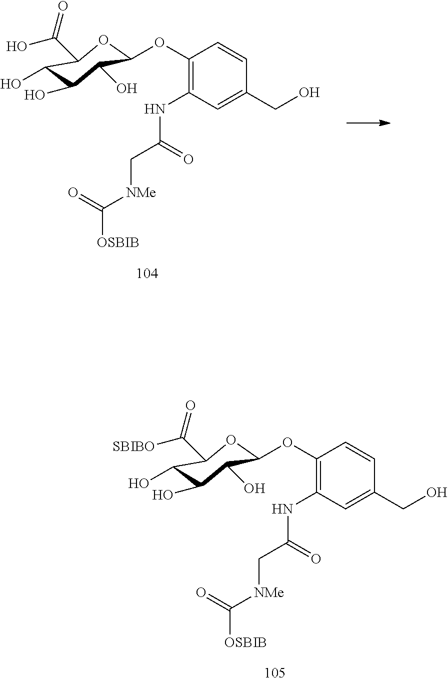

Compound 104 (295 mg, 0.469 mmol) was dissolved in anhydrous pyridine (5 mL) and cooled to 0° C. BIBSOTf (0.392 mL, 1.41 mmol) was added dropwise over 15 minutes checking by UPLC-MS for completion after addition of each stoichiometric equivalent. Reaction was diluted with EtOAc (100 mL), washed with 1M HCl (3×100 mL), dried MgSO₄, filtered and concentrated in vacuo. The crude product was purified by column chromatography 50 G KP-Sil, 10-100% EtOAc in Hex. Fractions containing the desired product concentrated to afford compound 105 as a colorless solid (301 mg, 0.363 mmol, 77%). LC-MS (Method C): t_{R}=1.65 min; MS (m/z) [M+H]⁺ calc. for C₄₁H₇₅N₂O₁₁Si₂ 827.49, found 827.31.

### Synthetic Example 73

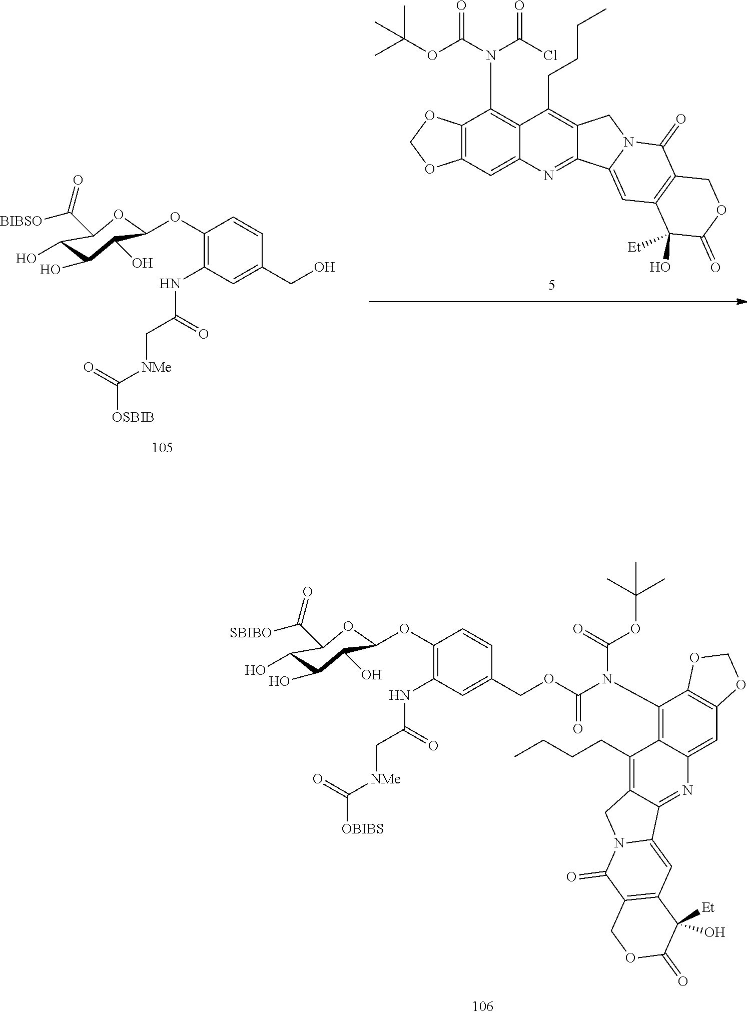

Compound 105 (218 mg, 0.264 mmol) was dissolved in anhydrous DCM (1 mL) and cooled to 0° C. The compound 5 (0.087 mmol) reaction solution from Example 2 was added directly to the DCM reaction solution. Reaction mixture allowed to warm to room temperature over 1 h. Stirred at room temperature for 16 hours. Reaction quenched with MeOH, then purified by flash chromatography 50 G KP-Sil 0-10% MeOH in DCM. Fractions containing the desired product were concentrated in vacuo to afford compound 106 as a yellow solid (66.1 mg, 0.0467 mmol, 53%). LC-MS (Method C): t_{R}=1.76 min; MS (m/z) [M+H]⁺ calc. for C₇₂H₁₀₆N₅O₂OSi₂ 1416.70, found 1416.74.

### Synthetic Example 74

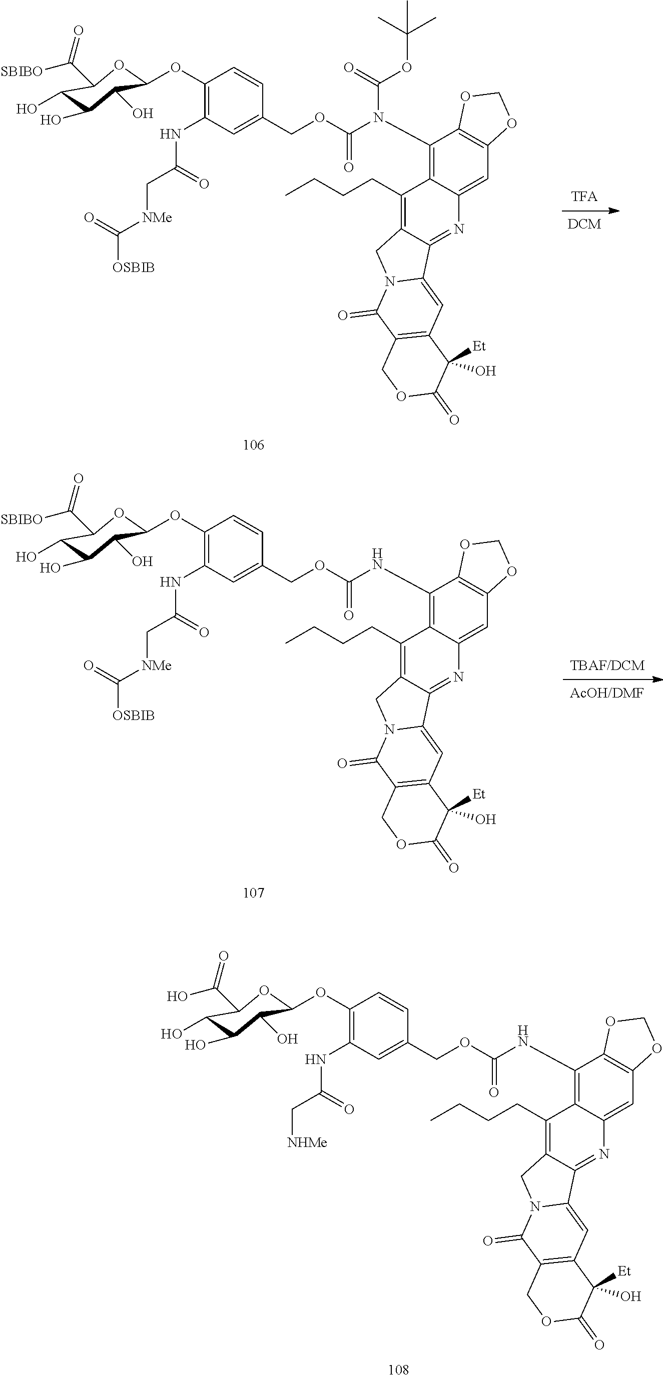

Compound 106 (66.1 mg, 0.0467 mmol) was dissolved in DCM (2 mL). TFA (0.4 mL) was added. The reaction was stirred for 20 minutes. Complete conversion observed by UPLC-MS. The reaction was concentrated in vacuo to afford compound 107 as a yellow solid and used in the next step without further purification. LC-MS (Method A): t_{R}=1.68 min; MS (m/z) [M+H]⁺ calc. for C₆₇H₉₈N₅O₁₈Si₂ 1316.64, found 1316.80.

Crude compound 107 (0.0467 mmol) was dissolved in anhydrous DMF (1 mL). AcOH (200 μL) was added followed by TBAF 1M in THE (200 uL). The reaction was stirred at room temperature for 30 minutes. Complete conversion was observed by UPLC-MS. Silica (˜100 mg) added to quench fluoride anion. The reaction mixture was filtered through a syringe filter, rinsed 2×1 mL 2:1 DMA:H₂O 10% AcOH and purified by prep-HPLC 30 mm 10-95% MeCN in H₂O 0.05% TFA. Fractions containing the desired product were lyophilized to afford compound 108 as a yellow powder (33.5 mg, 0.0383 mmol, 82%). LC-MS (Method A): t_{R}=1.16; MS (m/z) [M+H]⁺ calc. for C₄₂H₄₅N₅O₁₆ 875.29, found 875.82.

### Synthetic Example 75

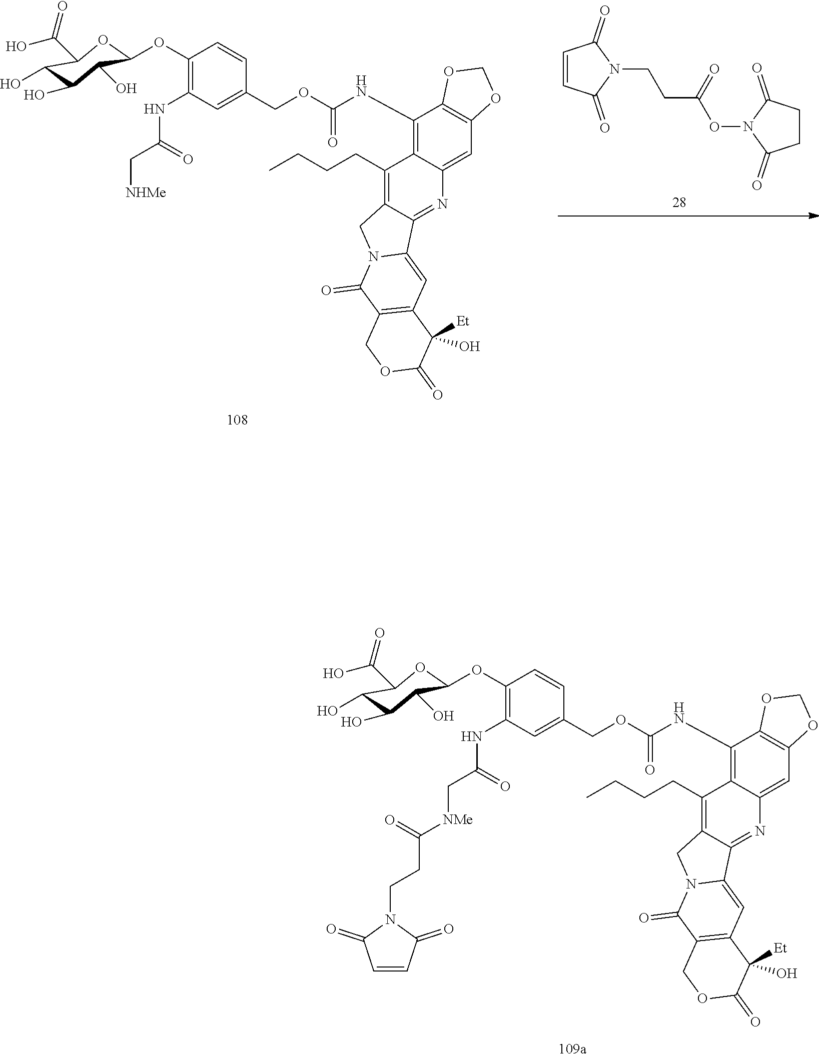

Compound 108 (33.5 mg, 0.0383 mmol) was dissolved in DMF. DIPEA (0.040 mL, 0.23 mmol) was added to the reaction followed by 3-(maleimido)propionic acid N-hydroxysuccinimide ester (28, 30.6 mg, 115 mmol). The reaction was stirred for 90 minutes. Complete conversion was observed by UPLC-MS. The reaction was quenched with AcOH, and purified by Prep-HPLC-21 mm. Fractions containing the desired product were lyophilized to afford compound 109a as a yellow powder (26.2 mg, 0.0255 mmol, 67%). LC-MS (Method A): t_{R}=1.39 min; MS (m/z) [M+H]⁺ calc. for C₄₉H₅₁N₆O₁₉ 1027.32, found 1026.88.

Similar procedures are used with any of compounds 18a-18p of Example 10 to prepare Drug Linker compounds of general formula 109

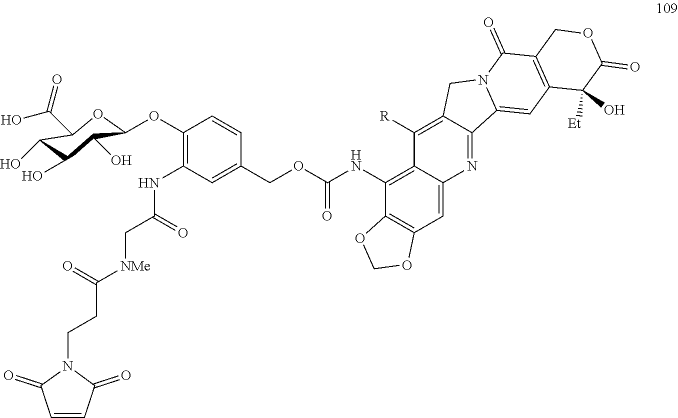

wherein R is any one of the R groups provided in compounds 18a-18p of Example 10, including the synthesized compound in which R is n-pentyl (compound 109b) starting from compound 18q.

### Synthetic Example 76

Compound 113 is prepared according to the following reaction scheme.

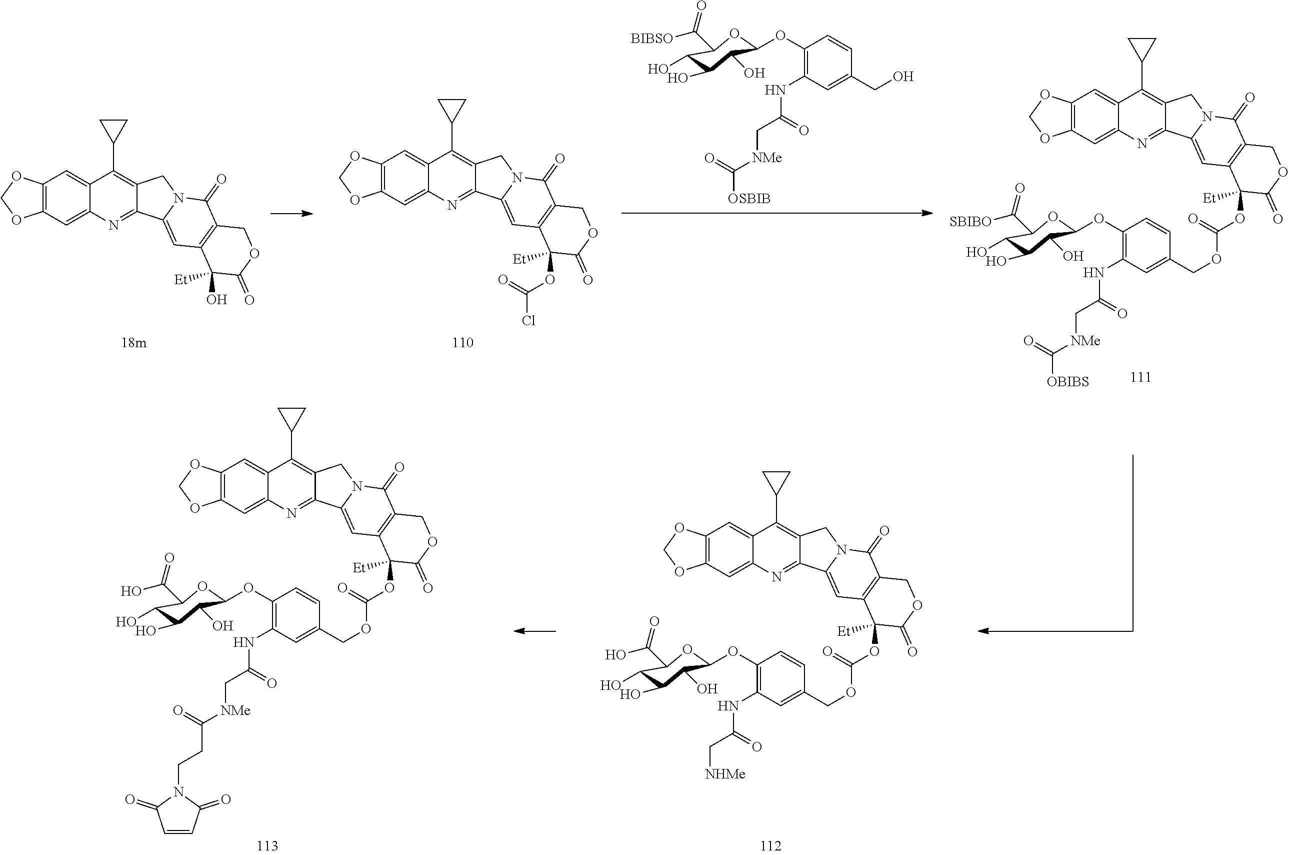

Similar procedures are used with any of compounds 18a-18p of Example 10 to prepare Drug Linker compounds of general formula 114:

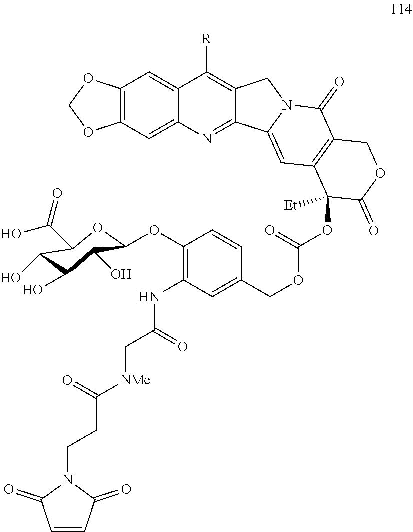

wherein R is any one of the R groups provided in compounds 18a-18p of Example 10, including those compounds that were synthesized in which R is n-pentyl (compound 114a) starting from compound 18h and R is n-butyl (compound 114b) starting from compound 6.

### Synthetic Example 77

Following the reaction scheme of Example 76 and Examples 44 and 45 compounds of formula 115 are prepared:

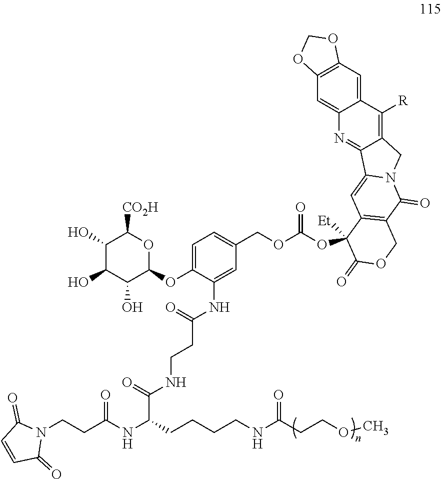

wherein subscript n is an integer from 4 to 24 and R is any one of the R groups provided in compounds 18a-18 of Example 10. Synthetic Example 78

Following the procedures of Examples 26-28, compounds of formula 116 are prepared:

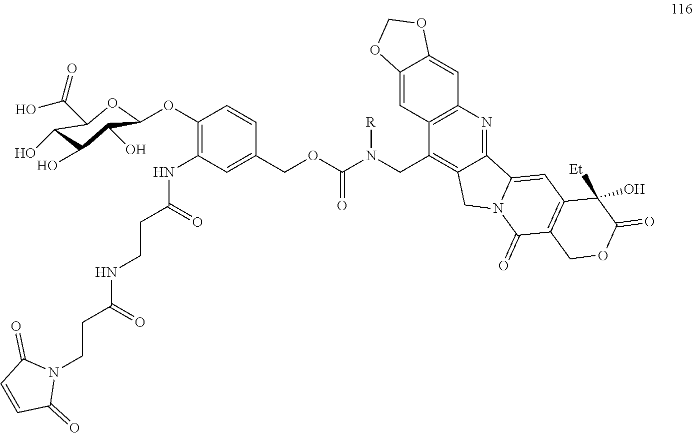

wherein R is any of the groups in camptothecin compounds 14a-14z of Example 7 that are compatible with the coupling reaction of compound 45 in so far as no reactive nucleophillic groups are present in the R substituent.

### Synthetic Example 79

Following the procedures of Example 14 and Examples 73-75, the compound of formula 117 is prepared:

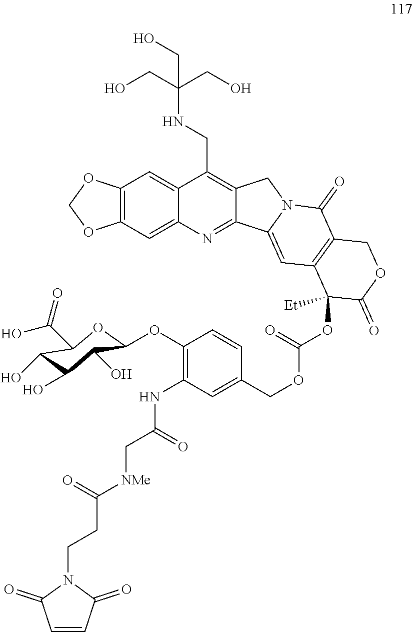

### Synthetic Example 80

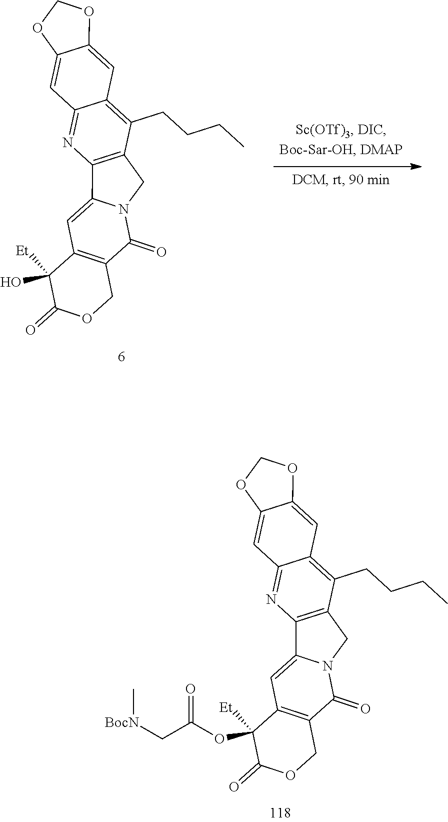 [SMILES: CCCCc1c2c(nc3cc4c(cc13)OCO4)-c1cc3c(c(=O)n1C2)COC(=O)[C@]3(O)CC ; CCCCc1c2c(nc3cc4c(cc13)OCO4)-c1cc3c(c(=O)n1C2)COC(=O)[C@@]3(CC)OC(=O)CN(C)C(=O)OC(C)(C)C]

Compound 6 (19.0 mg, 0.0424 mmol), prepared according to the procedure of Bioconjugate Chem. (2009) 20: 1242-1250, was dissolved in anhydrous DCM (0.5 mL). DMAP (15.4 mg, 0.127 mmol), Sc(OTf)₃ (12.5 mg, 0.0254 mmol), Boc-Sar-OH (24.1 mg, 127 mmol) and DIC (21 μL, 136 mmol) were added to the reaction. The reaction was stirred for 90 minutes. The reaction was purified by column chromatography 0-5% MeOH in DCM. Fractions containing the desired product were concentrated in vacuo to afford compound 2 as yellow solid. LC-MS (Method A): t_{R}=1.52 min; MS (m/z) [M+H]⁺ calc. for C₃₃H₃₈N₃O₉ 620.26, found 619.96.

### Synthetic Example 81

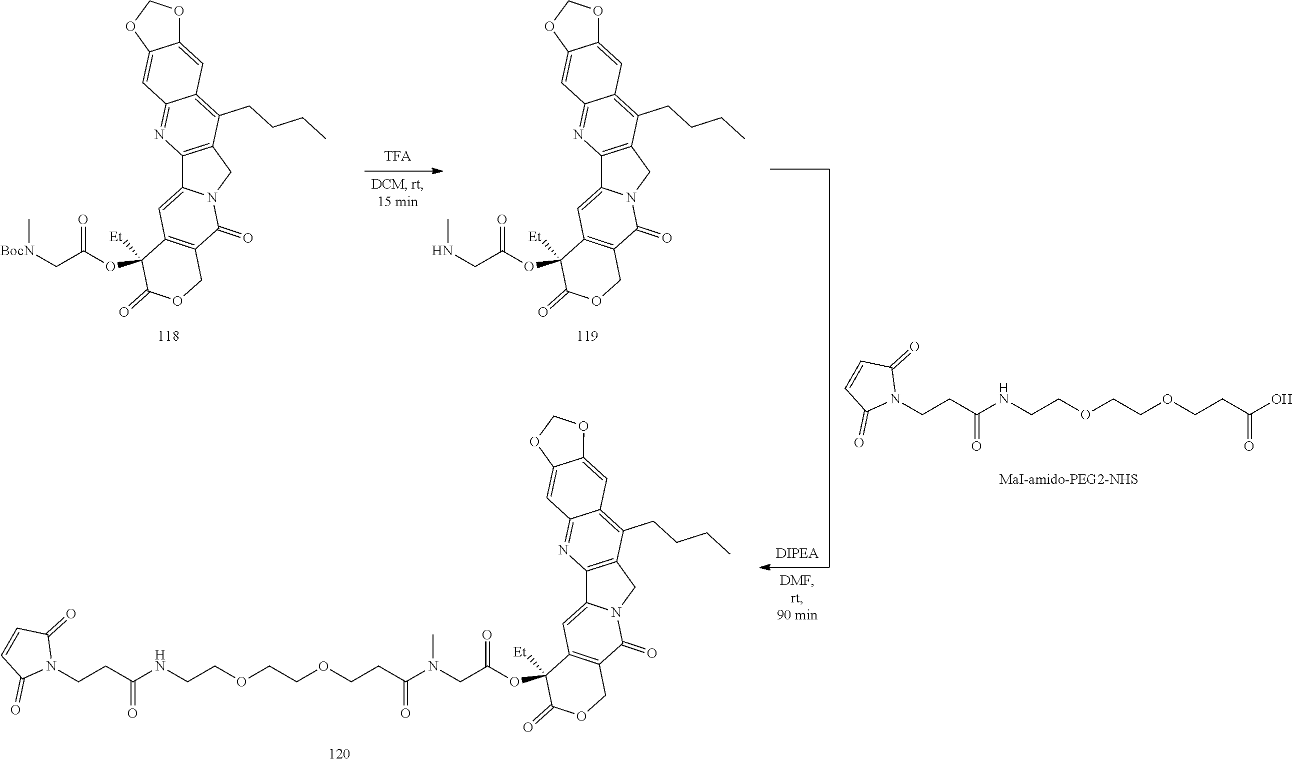 [SMILES: CCCCc1c2c(nc3cc4c(cc13)OCO4)-c1cc3c(c(=O)n1C2)COC(=O)[C@@]3(CC)OC(=O)CN(C)C(=O)OC(C)(C)C ; CCCCc1c2c(nc3cc4c(cc13)OCO4)-c1cc3c(c(=O)n1C2)COC(=O)[C@@]3(CC)OC(=O)CNC ; CCCCc1c2c(nc3cc4c(cc13)OCO4)-c1cc3c(c(=O)n1C2)COC(=O)[C@@]3(CC)OC(=O)CN(C)C(=O)CCOCCOCCNC(=O)CCN1C(=O)C=CC1=O ; O=C(O)CCOCCOCCNC(=O)CCN1C(=O)C=CC1=O]

Compound 118 (25.2 mg, 0.0407 mmol) was dissolved in 20% TFA in DCM (2 mL). The reaction was stirred for 15 minutes at which point complete conversion was observed by UPLC-MS. The reaction was concentrated in vacuo to afford compound 119 as a yellow solid used in the next step without further purification. LC-MS (Method A): t_{R}=0.93 min; MS (m/z) [M+H]⁺ calc. for C₂₈H₃₀N₃O₇ 520.21, found 519.87.

The crude product 119 (0.0407 mmol) was dissolved in anhydrous DMF (0.5 mL). DIPEA (35 μL, 0.203 mmol) was added followed by Mal-amido-PEG2-NHS (52 mg, 0.122 mmol) obtained from Broadpharm (CAS: 955094-26-5). The reaction was stirred for 90 minutes, quenched with AcOH (50 μL), and purified by Prep-HPLC. Fractions containing the desired product were lyophilized to afford compound 120 as a yellow powder (18.9 mg, 0.0228 mmol, 56%). LC-MS (Method A): t_{R}=1.16 min; MS (m/z) [M+H]⁺ calc. for C₄₂H₄₈N₅O₁₃ 830.32, found 829.86. Compound 120 is an exemplary Drug Linker compound of general formula Z′-A-S*—W-CPT2

### Synthetic Example 82

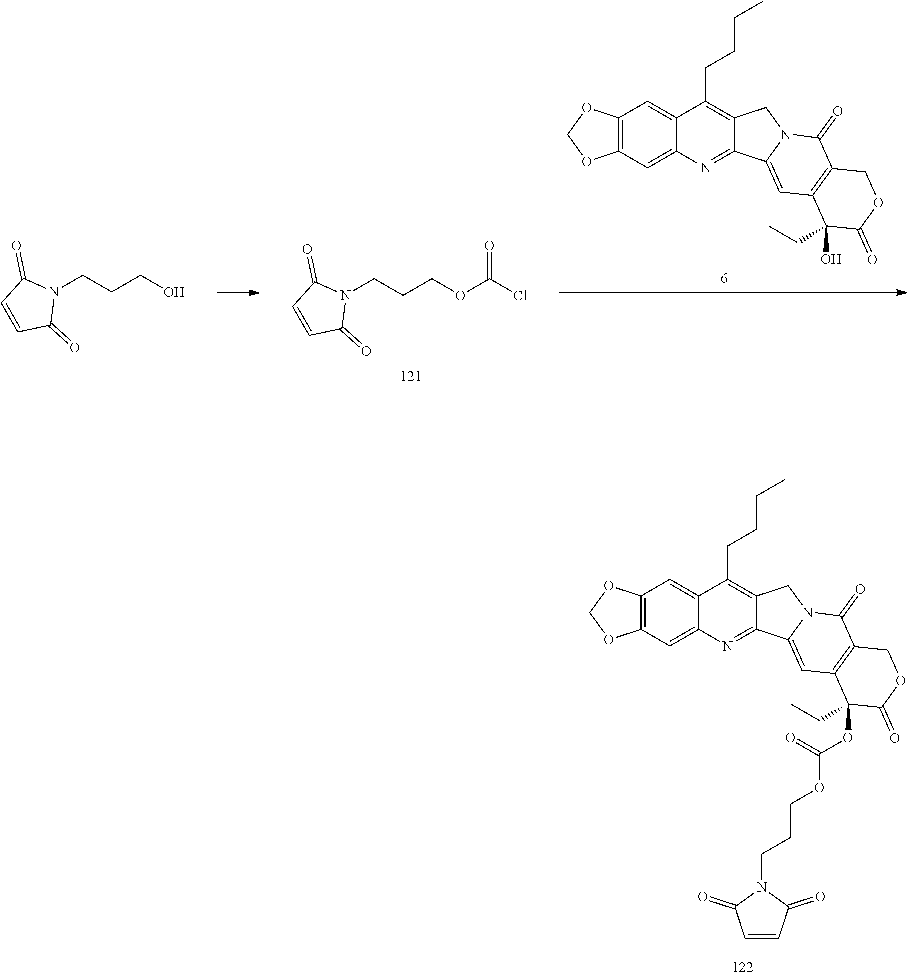 [SMILES: O=C1C=CC(=O)N1CCCO ; O=C(Cl)OCCCN1C(=O)C=CC1=O ; CCCCc1c2c(nc3cc4c(cc13)OCO4)-c1cc3c(c(=O)n1C2)COC(=O)[C@]3(O)CC ; CCCCc1c2c(nc3cc4c(cc13)OCO4)-c1cc3c(c(=O)n1C2)COC(=O)[C@@]3(CC)OC(=O)OCCCN1C(=O)C=CC1=O]

N-(3-Hydroxypropyl)maleimide (455 mg, 2.93 mmol) was dissolved in anhydrous DCM (4 mL). Phosgene 20% w/w in toluene was added and reaction was stirred for 60 minutes and then concentrated under flow of nitrogen, followed by concentration in vacuo to provide crude compound 122, which was reconstituted in 50 mg/mL in DCM and used directly in the next step.

Compound 6 (10 mg, 0.022 mmol), prepared according to the procedure of Bioconjugate Chem. (2009) 20: 1242-1250, was dissolved in anhydrous DCM (0.5 mL). DMAP (3 mg, 0.02 mmol) was added to the reaction. The compound 64 chloroformate solution (1 mL) prepared in the previous step was added to the reaction. The reaction was stirred for 90 minutes. Approximately 50% conversion to the desired product was observed. The reaction was purified by FCC 10 G Biotage Ultra 0-5% MeOH in DCM. Fractions containing the desired product were concentrated in vacuo to afford compound 65 as a yellow solid (7.5 mg, 0.012 mmol, 53%). LC-MS (Method A): t_{R}=2.17 min; MS (m/z) [M+H]⁺ calc. for C₃₃H₃₂N₃O₁₀ 630.21, found 629.98. Compound 122 is an exemplary Drug Linker compound of general formula Z′-A-CPT2

### Synthetic Example 83

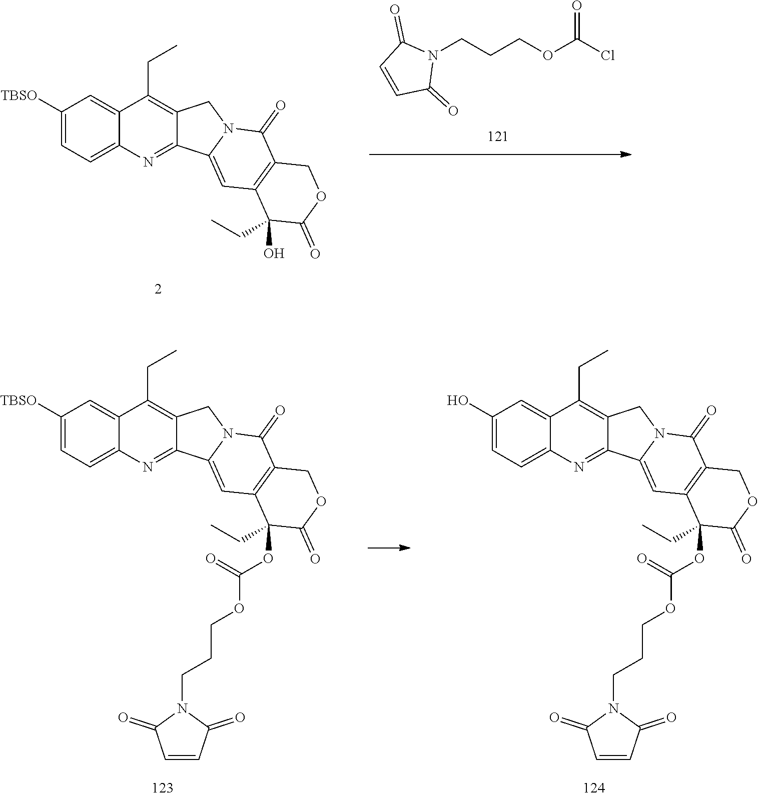 [SMILES: CCc1c2c(nc3ccc(O[Si](C)(C)C(C)(C)C)cc13)-c1cc3c(c(=O)n1C2)COC(=O)[C@]3(O)CC ; O=C(Cl)OCCCN1C(=O)C=CC1=O ; CCc1c2c(nc3ccc(O[Si](C)(C)C(C)(C)C)cc13)-c1cc3c(c(=O)n1C2)COC(=O)[C@@]3(CC)OC(=O)OCCCN1C(=O)C=CC1=O ; CCc1c2c(nc3ccc(O)cc13)-c1cc3c(c(=O)n1C2)COC(=O)[C@@]3(CC)OC(=O)OCCCN1C(=O)C=CC1=O]

Compound 2 (45 mg, 0.088 mmol), prepared according to the procedure of Example 1, was dissolved in anhydrous DCM (0.5 mL). DIPEA (0.05 mL) and DMAP (11 mg, 0.09 mmol) were added to the reaction. The compound 64 chloroformate solution (1.1 mL) previously described was added to the solution and the reaction was stirred for 60 minutes. Approximately 70% conversion to the desired product was observed. The reaction was quenched with MeOH, then filtered through silica 10% MeOH in DCM. The eluent was concentrated to afford compound 123 as a white solid (0.088 mmol) which was used in the next step without further purification. LC-MS (Method B): t_{R}=1.91 min; MS (m/z) [M+H]⁺ calc. for C₃₆H₄₂N₃O₉Si 688.27, found 687.99.

Crude compound 123 (0.088 mmol) was dissolved in DMF (2 mL). AcOH (0.5 mL) was added to the reaction mixture followed by TBAF 1M in THE (0.440 mL, 0.444 mmol). The reaction was stirred for 30 minutes, then purified by Prep-HPLC 21 mm 5-95% MeCN in H₂O 0.05% TFA. Fractions containing the desired product were lyophilized to afford compound 124 as a yellow powder (10.24, 0.01785 mmol, 20%). LC-MS (Method A): t_{R}=1.24 min; MS (m/z) [M+H]⁺ calc. for C₃₀H₂₈N₃O₉ 574.18, found 573.90. Compound 124 is an exemplary Drug Linker compound of general formula Z′-A-CPT3.

### Synthetic Example 84

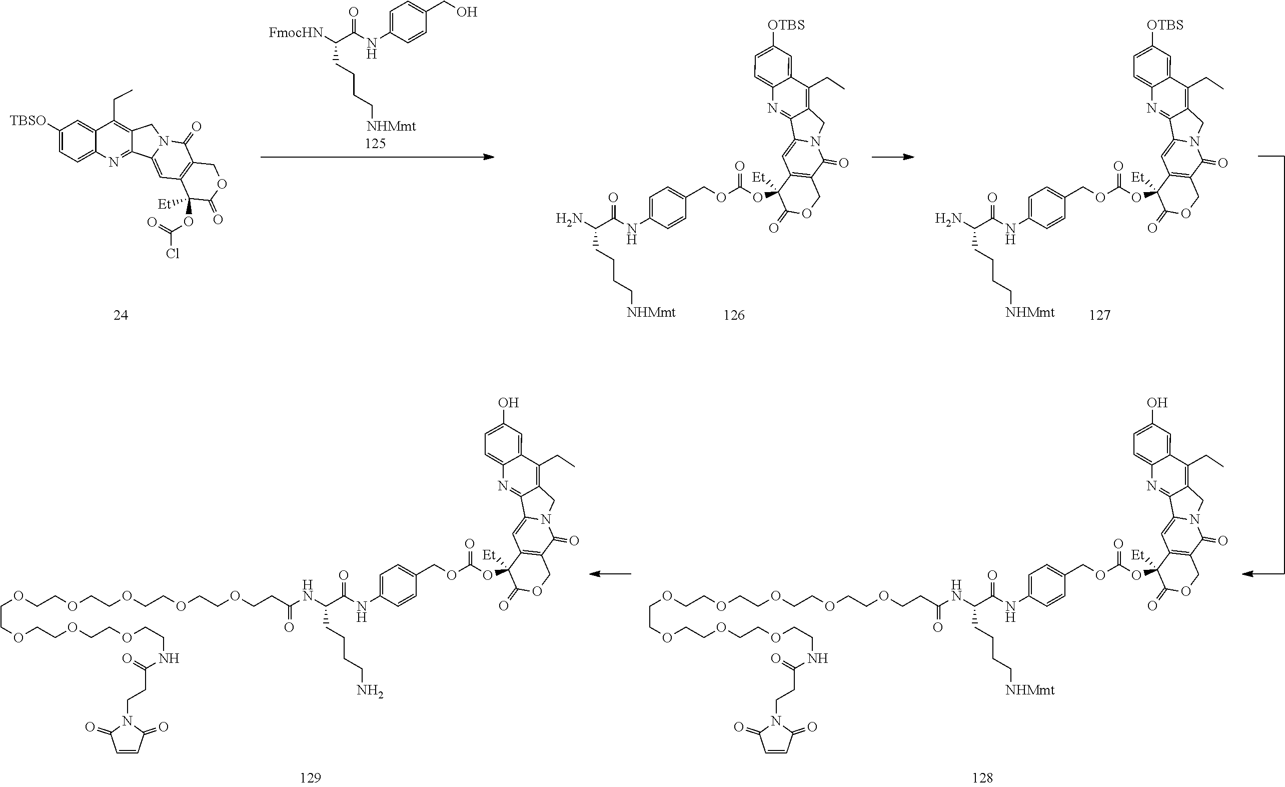

To the compound 24 chloroformate reaction mixture prepared according to Example 14 was added solid compound 125 (291 mg, 0.390 mmol), prepared according to the procedure of Bioorganic & Medicinal Chemistry Letters (2002) 12: 217-219. Conversion to product was observed after 10 minutes by UPLC-MS. The reaction was quenched MeOH, concentrated in vacuo, then roughly purified by column chromatography 0-10% MeOH in DCM. Fractions containing the desired product with impurities (free drug, linker) were concentrated to afford compound 126 as a slight yellow solid, which was used in the next step without further purification. LC-MS (Method C): t_{R}=1.85 min; MS (m/z) [M+H]⁺ calc. for C₇₇H₈₀N₅O₁₁Si 1278.56, found 1278.09.

Crude compound 126 was dissolved in 50% Et₂NH in DCM. The reaction was stirred for 30 minutes at which time complete Fmoc deprotection was observed by UPLC-MS. The reaction mixture was concentrated in vacuo. After evaporation complete deprotection of the TBS protecting group was observed. The reaction was purified by column chromatography 0-10% MeOH in DCM. Fractions containing the desired product and minor impurities were concentrated in vacuo to afford compound 127 as an off white solid used (100 mg, 0.10 mmol, 41%) in next step without further purification. Rt=1.03 min Hydrophobic Method UPLC. MS (m/z) [M+H]⁺ calc. for C₅₆H₅₆N₅O₉ 942.41, found 942.18.

Crude compound 127 (100 mg, 0.10 mmol) was dissolved in anhydrous DMF (2 mL). DIPEA (0.037 mL, 0.212 mmol) was added followed by MP-PEG8-OSu (81 mg, 117 mmol). Complete conversion was observed by UPLC-MS after 5 minutes. The reaction was quenched with MeOH and AcOH, concentrated in vacuo to provide crude compound 128, which was used in the next step without further purification.

Crude compound 128 was dissolved in 20% TFA in DCM and stirred for 1 hour. The reaction was concentrated in vacuo and purified by prep-HPLC. Fractions containing the desired product were lyophilized to afford compound 129 as an off-white solid (31.0 mg, 0.0249 mmol, 23%). LC-MS (Method A): t_{R}=1.37 min; MS (m/z) [M+H]⁺ calc. for C₆₂H₈₂N₇O₂₀ 1244.56, found 1243.93. Compound 129 is an exemplary Drug Linker compound of general formula Z′-A-S*—W-RL-CPT3

### Synthetic Example 85

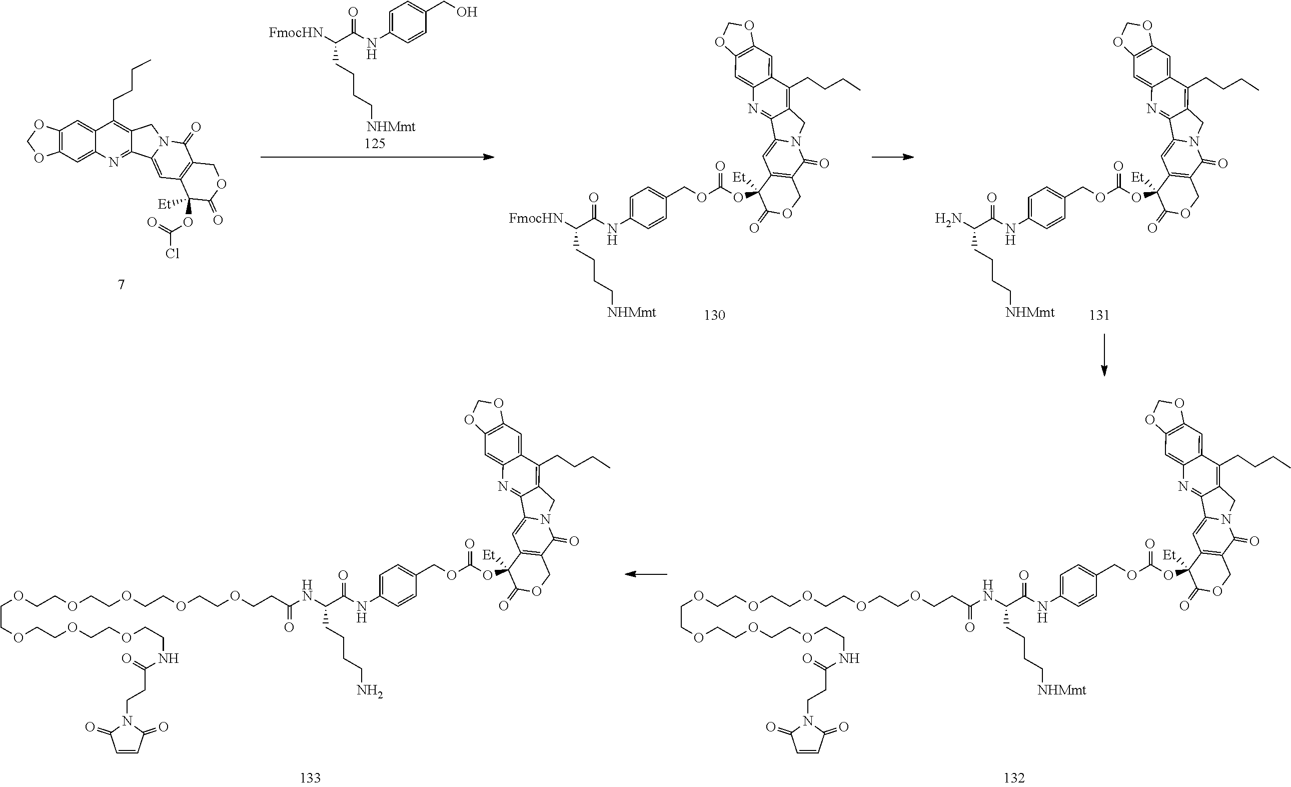

To the compound 7 chloroformate solution (0.334 mmol) from Example 3 was added compound 125 (372 mg, 0.499 mmol), prepared according to the procedure of Bioorganic & Medicinal Chemistry Letters (2002) 12: 217-219, in one portion. The reaction was stirred for 45 minutes. Using sample preparation previously described nearly complete conversion of the chloroformate to the desired product was observed. The reaction was quenched with MeOH, concentrated in vacuo and purified by FCC 50 G KP-Sil 0-5% MeOH in DCM using a step gradient. Fractions containing the desired product and mixture of impurities concentrated in vacuo to afford compound 130 as a yellow solid (450 mg, ˜80% w/w, 0.294 mmol), which was used in next step without further purification. LC-MS (Method A): t_{R}=1.61 min; MS (m/z) [M+H]⁺ calc. for C₇₄H₇₀N₅O₁₂ 1220.50, found 1220.16.

Crude compound 130 (0.294 mmol) was dissolved in 10 mL 50% Et₂HN in DCM. The reaction was stirred for 30 minutes at which time nearly complete conversion was observed by UPLC-MS. The reaction was concentrated in vacuo to afford compound 131 as a yellow solid, which was used in next step without further purification. LC-MS (Method A): t_{R}=1.20 min; MS (m/z) [M+H]⁺ calc. for C₅₉H₆₀N₅O₁₀ 998.43, found 998.26.

Crude compound 131 (0.294 mmol) from the previous step was dissolved in anhydrous DCM (5 mL). MP-Peg8-OSu (483 mg, 0.701 mmol) dissolved in DMF (250 mg/mL) was added. DIPEA (0.3 mL) was added and the reaction was stirred for 30 minutes at which time complete conversion to compound 132 was observed by UPLC-MS. The reaction mixture containing compound 132 was used in the next step without further purification. LC-MS (Method A): t_{R}=1.37 min; MS (m/z) [M+H]⁺ calc. for C₈₅H₁₀₂N₇O₂₂ 1572.71, found 1571.90.

The reaction mixture (compound 57, 0.294 mmol) containing crude compound 132 was quenched and acidified with TFA (1 mL). The reaction was stirred at room temperature for 20 minutes at which time complete conversion was observed. The reaction was concentrated in vacuo and purified by Prep-HPLC 30 mm C18 10-95% MeCN in H₂O 0.05% TFA. Fractions containing the desired product were lyophilized to afford compound 133 as a yellow solid (107 mg, 0.0823 mmol, 28%). LC-MS (Method A): t_{R}=1.17 min; MS (m/z) [M+H]⁺ calc. for C₆₅H₈₆N₇O₂₁ 1300.59, found 1300.69. Compound 133 is an exemplary Drug Linker compound of general formula Z′-A-S*—W-RL-CPT2.

### Synthetic Example 86

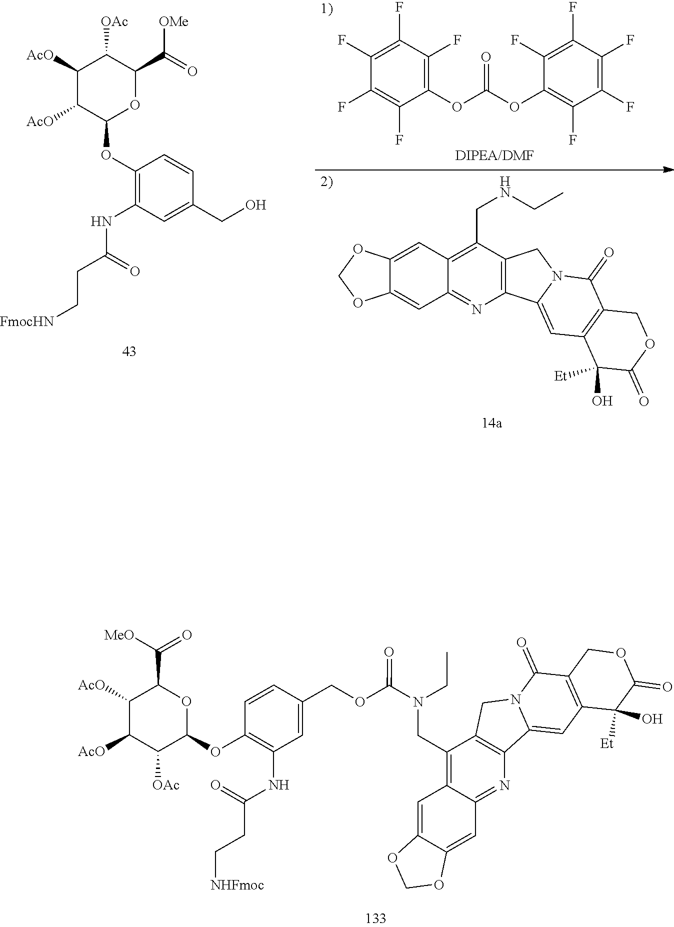

Compound 43 (143.4 mg, 0.1916 mmol), prepared according to the procedure Bioconjugate Chem. (2006) 17: 831-840 was dissolved in anhydrous DMF (0.5 mL). DIPEA (0.0334 mL, 0.192 mmol). To the DMF solution was added bis-(pentafluorophenyl) carbonate (75.5 mg, 0.192 mmol, purchased form TCI America Product Number B3604). The reaction was stirred for 30 minutes, followed by addition of compound 14a (28.7 mg, 0.0639 mg) from Example 7 in 0.5 mL anhydrous DMF. The reaction was stirred for 2 hours at room temperature. Complete conversion was observed by UPLC-MS. The reaction was quenched with AcOH (0.035 mL), then purified by preparative HPLC on a 21.2×250 mm Max-RP column using a gradient of 30-95% MeCN in H₂O 0.05% TFA. Fractions containing the desired product was concentrated in vacuo to afford compound 133 as a yellow solid. (76.6 mg, 0.0623 mmol, 98%). LC-MS (Method F): t_{R}=1.68 min; MS (m/z) [M+H]+ calc. for C₆₃H₆₂N₅O₂₁ 1224.39, found 1224.46.

### Synthetic Example 87

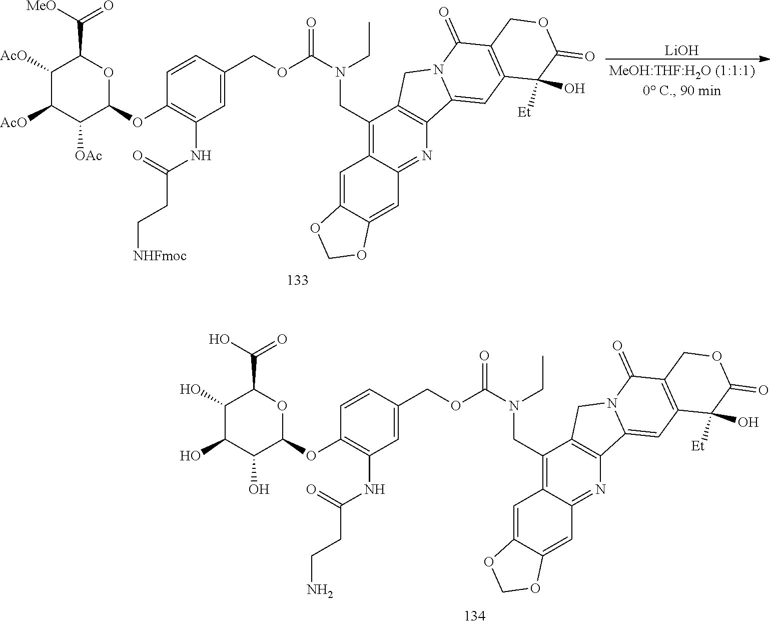

Compound 133 (76.6 mg, 0.0623 mmol) was dissolved in THF:MeOH 1:1 (2 mL). The reaction was cooled with an ice/water bath. LiOH (45 mg, 1.9 mmol) was added and the reaction was stirred for 30 minutes. Conversion to the acetate deprotected product observed by UPLC-MS. H₂O (1 mL) was added to the reaction mixture. The reaction was stirred for 60 minutes. Complete conversion observed by UPLC-MS. The reaction was quenched with AcOH (0.2 mL), concentrated in vacuo and purified by preparative HPLC using a 21.2×250 mm Max-RP column eluted with a gradient of 5-40-95% MeCN in H₂O 0.05% TFA. Fractions containing the desired compound were concentrated in vacuo to afford compound 134 as a yellow solid (33.3 mg, 0.0386 mmol, 62%). LC-MS (Method D): t_{R}=1.09 min; MS (m/z) [M+H]+ calc. for C₄₁H₄₄N₅O₁₆ 862.28, found 862.16.

### Synthetic Example 88

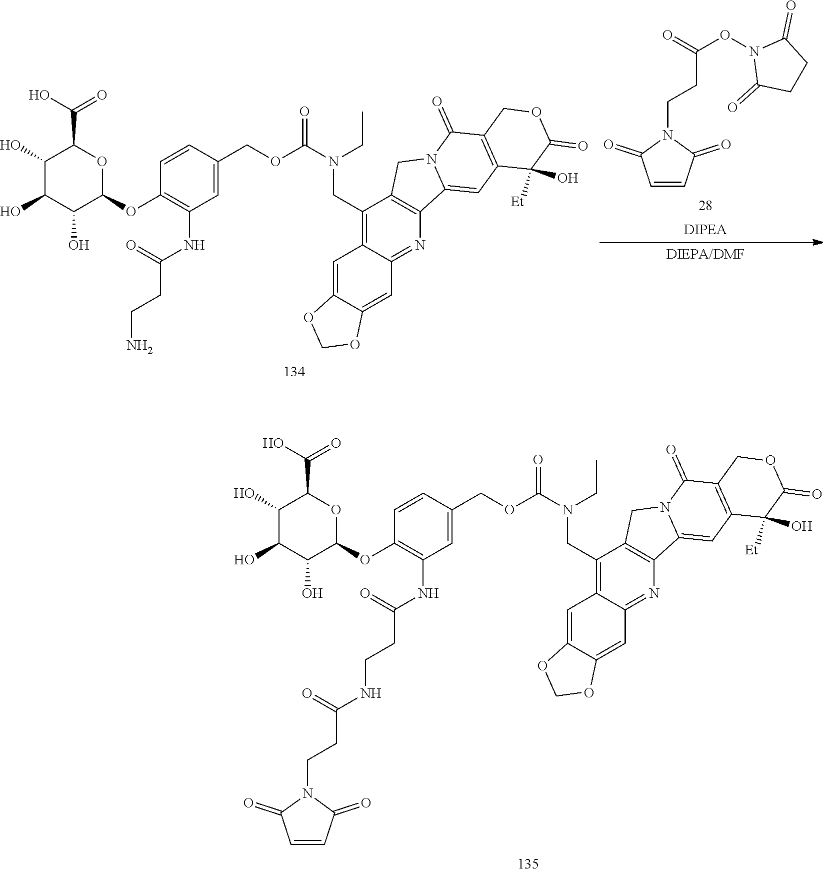

To compound 134 (33.3 mg, 0.0386 mmol) dissolved in anhydrous DMF (0.5 mL) and DIPEA (0.020 mL, 0.116 mmol) was added 3-(maleimido)propionic acid N-hydroxysuccinimide ester (28, 15.4 mg, 0.0580 mmol), purchased from TCI America (product number S0427). The reaction was stirred for 30 minutes. Complete conversion was observed after 5 minutes by UPLC-MS. The reaction was quenched with AcOH (0.020 mL) and purified by preparative HPLC eluting with 5-40-95% MeCN in H₂O 0.05% TFA on a 21.2×250 mm Max-RP. Fractions containing the desired product were lyophilized to afford compound 135 as a yellow powder (21.85 mg, 0.02157 mmol, 55.8%). LC-MS (Method D): t_{R}=1.27 min; MS (m/z) [M+H]+ calc. for C₄₈H₄₉N₆O₁₉ 1013.30, found 1013.38.

### Synthetic Example 89

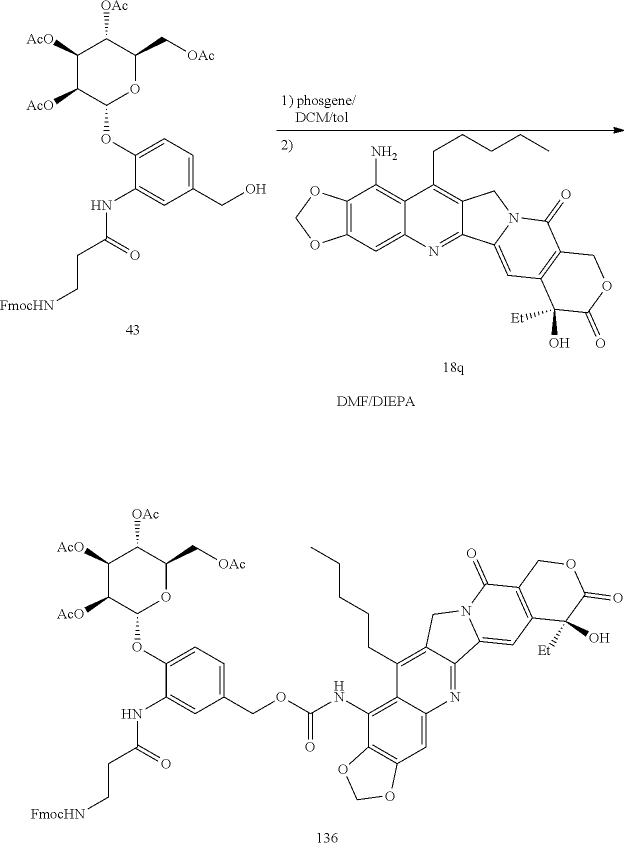

Compound 18q (100.0 mg, 0.2094 mmol) of Example 10 was dissolved in anhydrous DCM (4 mL). Phosgene 20% w/w in Toluene (2 mL, 3.51 mmol) was added to the reaction. The reaction was stirred for 2 h at which complete conversion to activated isocyanate intermediate was observed by quenching a 2 μL aliquot of the reaction 98 μL MeOH and observing the formed MeOH adduct by UPLC-MS. The reaction was concentrated under stream of nitrogen, then further dried under high vacuum. Compound 43 (239.6 mg, 0.3141 mmol) was dissolved in anhydrous DMF (1 mL), then added directly to the activated isocyanate solid. DIPEA (0.11 mL, 0.63 mmol) was added and the reaction was stirred to dissolve all components. The reaction was stirred for 30 minutes at which point complete conversion was observed. The reaction was quenched with MeOH, concentrate in vacuo, and purified by column chromatography eluting with 0-6% MeOH in DCM on a 25 G KP-Sil column. Fractions containing the desired product and drug related impurities were concentrated to afford compound 113 as a yellow solid (186.6 mg, 0.1474 mmol, 70%). The product was used in the next step without further purification. LC-MS (Method D): t_{R}=2.14 min; MS (m/z) [M+H]+ calc. for C₆₆H₆₈N₅O₂₁ 1266.44, found 1266.57.

### Synthetic Example 90

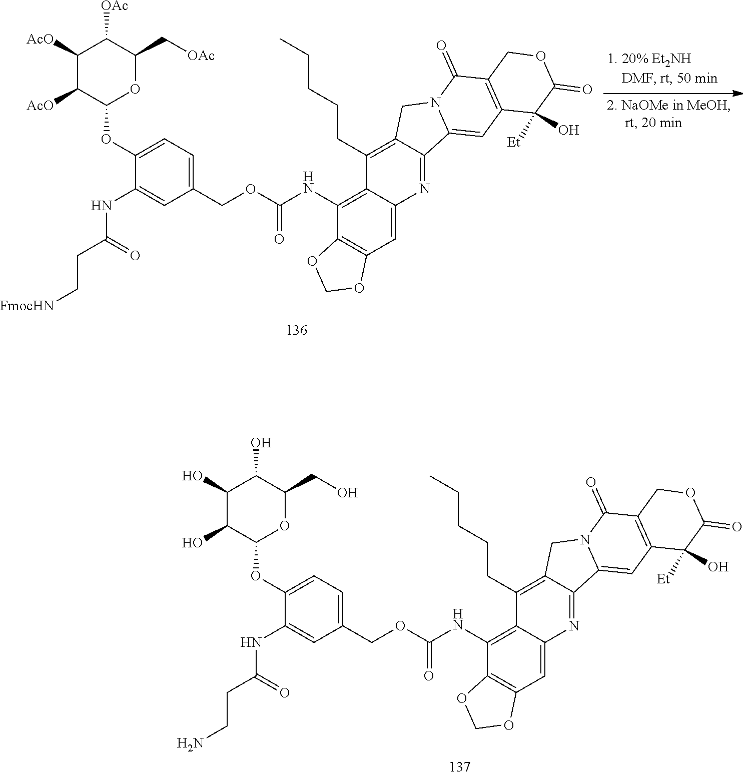

Compound 136 (186.6 mg, 0.1474 mmol) was dissolved in 20% diethylamine in DMF (2 mL). The reaction was stirred for 50 minutes at which point nearly complete deprotection of the Fmoc protecting group was observed. The reaction was concentrated in vacuo and re-dissolved in MeOH (2 mL). NaOMe (0.5 M in MeOH, 1.77 mL, 0.884 mmol) was added and the reaction was stirred at room temperature for 20 minutes. Complete conversion was observed by UPLC-MS after 20 minutes. The reaction was neutralized with AcOH, concentrated in vacuo and purified by preparative HPLC eluting with 5-40-95% MeCN in H₂O 0.05% TFA on a 21.2×250 mm Max-RP column. Fractions containing the desired product were concentrated in vacuo to afford compound 137 as a yellow solid (22.5 mg, 0.0257 mmol, 17%). LC-MS (Method D): t_{R}=1.13 min; MS (m/z) [M+H]+ calc. for C₄₃H₅₀N₅O₁₅ 876.33, found 876.22.

### Synthetic Example 91

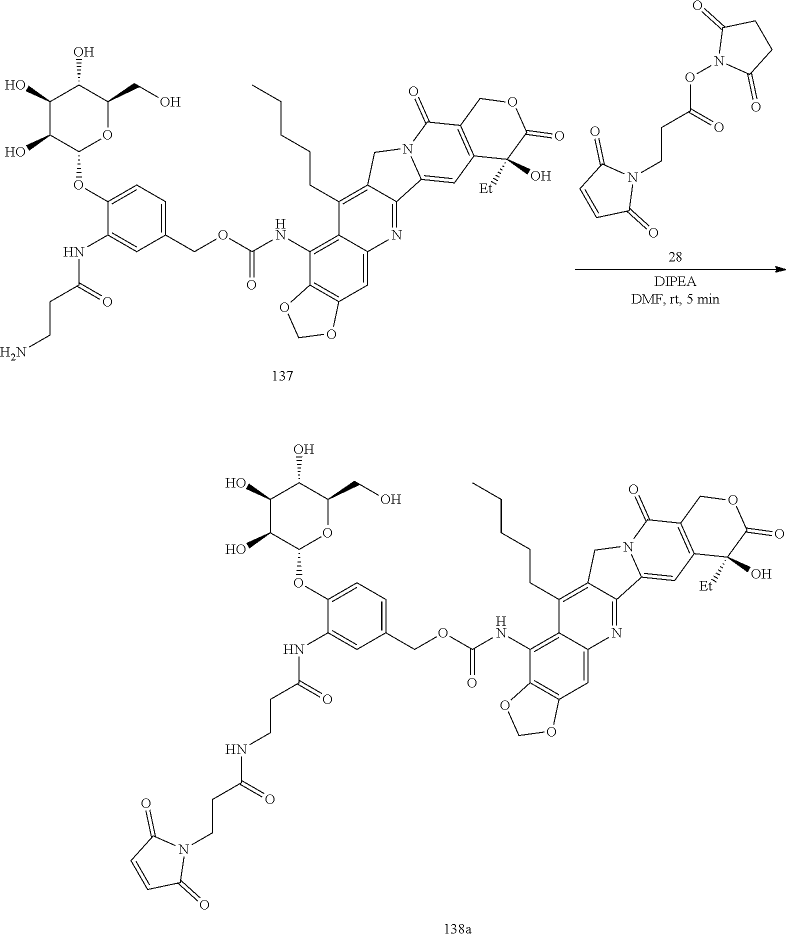

Compound 137 (22.5 mg, 0.0257 mmol) was dissolved in anhydrous DMF (0.5 mL). DIPEA (0.027 mL, 0.15 mmol) was added to the reaction followed by 3-(maleimido)propionic acid N-hydroxysuccinimide ester (28, 20.5 mg, 0.0771 mmol, purchased from TCI America product number S0427). The reaction was stirred for 5 minutes. Complete conversion was observed by UPLC-MS. The reaction was quenched with AcOH (0.030 mL) and purified by preparative HPLC eluting with 5-40-95% MeCN in H₂O 0.05% TFA on a 21.2×250 mm MaxRP column. Fractions containing the desired product were lyophilized to afford compound 138a as a yellow powder (11.36 mg, 0.01106 mmol, 43.1%). LC-MS (Method D): t_{R}=1.33 min; MS (m/z) [M+H]+ calc. for C₅₀H₅₅N₆O₁₈ 1027.36, found 1027.15.

### Synthetic Example 92

Following the procedures form Examples 88-90 Drug Linker compounds of general formula 138 are prepared:

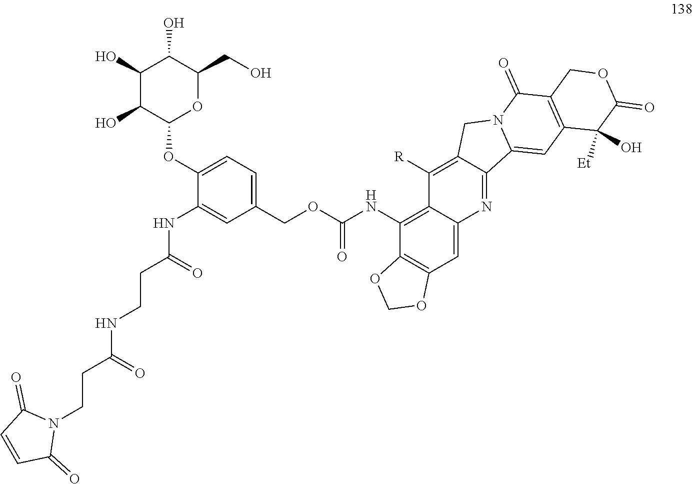

wherein R is any one of the R groups provided in compounds 18a-18p of Example 10, including the synthesized Drug Linker compound in which R is cyclopropyl (compound 138b) starting from compound 18r, except that the mannose residue moiety is replaced by glucuronic acid.

### Synthetic Example 93

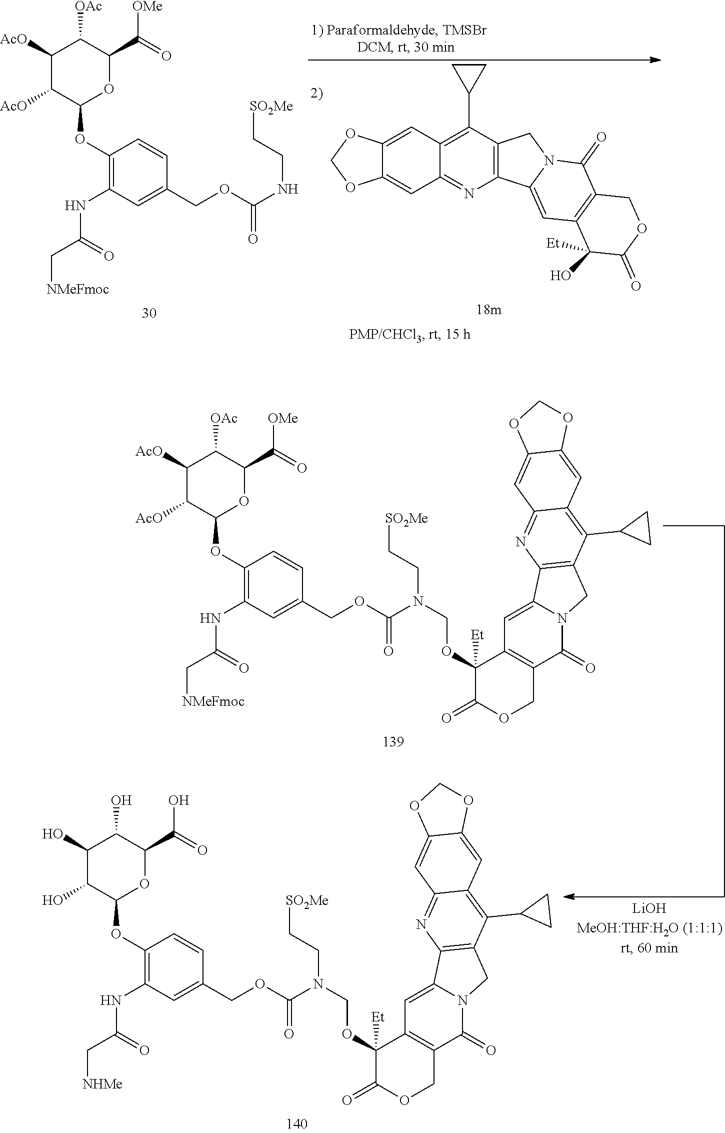

Compound 30 (623 mg, 0.694 mmol), prepared as described by Examples 21 and 22, was dissolved in anhydrous DCM (4 ml). Paraformaldehyde (208 mg, 6.94 mmol) was added to the reaction followed by TMSBr (0.12 mL, 0.925 mmol). The reaction was stirred at room temperature for 30 minutes at which point complete conversion to the activated chloromethyl intermediate was observed by UPLC-MS. The reaction was filtered through a syringe filter, rinsed with DCM 2 mL, and toluene (2 mL) was added to azeotrope the final mixture. The eluent was concentrated in vacuo to afford a colorless solid. Compound 18m (100.0 mg, 0.2313 mmol) from Example 10 was azeotroped with toluene. The chloromethyl intermediate was dissolve in anhydrous CHCl₃ (6 mL) and added directly compound 18m followed by 1,2,2,6,6-pentamethylpiperidine (PMP, 0.17 mL, 0.93 mmol). The reaction was stirred at room temperature for 15 hours, quenched MeOH, and concentrated in vacuo. The crude reaction mixture containing compound 139 was used in the next step without purification.

Crude compound 139 (0.2313 mmol) was dissolved in 1:1 MeOH:THF (4 mL). LiOH (55.4 mg, 2.31 mmol) was added and the reaction was stirred for 30 minutes. H₂O (2 mL) was added and the reaction was stirred for 30 minutes. The reaction was quenched with AcOH (0.1 mL), concentrated in vacuo and purified by reverse phase flash column chromatography using a Biotage Ultra C18 60 G column eluting with a gradient of 5-30-95% MeCN in H₂O 0.1% formic acid. Fractions containing the desired product and impurities were concentrated and re-purified by preparative HPLC using a 21.2×250 mm Max-RP column with a gradient of 5-30-95% MeCN in H₂O 0.1% formic acid. Fractions containing the desired product were concentrated in vacuo to afford compound 140 as a yellow solid (40.9 mg, 0.0417 mmol, 18%). LC-MS (Method D): t_{R}=1.23 min; MS (m/z) [M+H]⁺ calc. for C₄₅H₅₀N₅O₁₈S 980.29, found 980.20.

### Synthetic Example 74

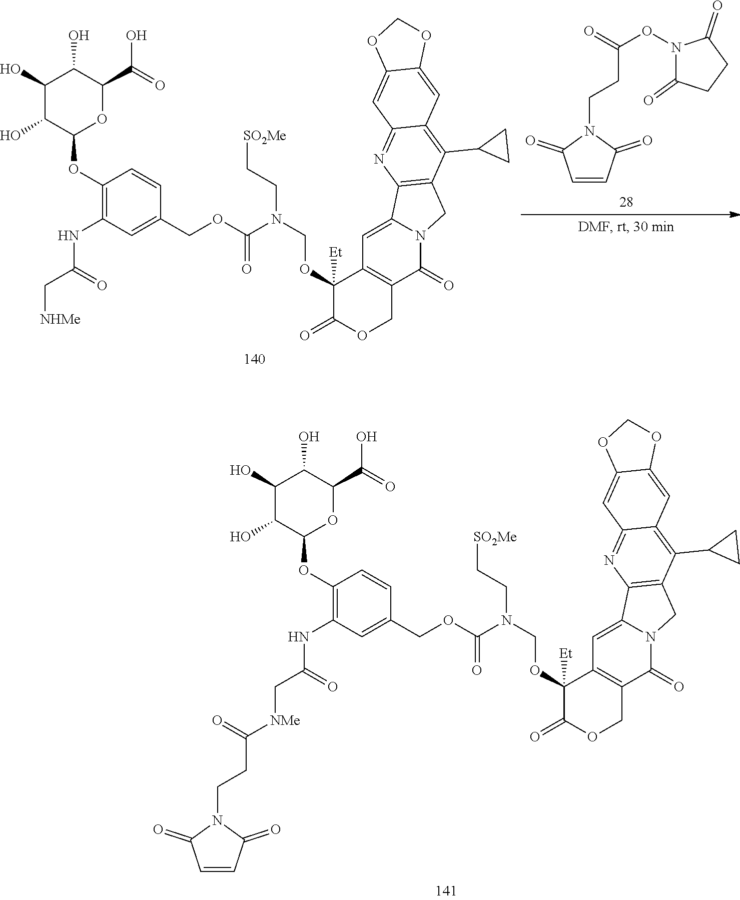

Compound 140 (40.9 mg, 0.0417 mmol) was dissolved in anhydrous DMF (1 mL). DIPEA (43 μL, 0.25 mmol) was added followed by 3-(maleimido)propionic acid N-hydroxysuccinimide ester (28, 33.3 mg, 0.125 mmol), purchased from TCI America product number S0427). The reaction was stirred for 30 minutes. Complete conversion was observed by UPLC-MS. The reaction was quenched with AcOH (5 μL) and purified by preparative HPLC eluting with 5-40-95% MeCN in H₂O 0.1% formic acid on a 21.2×250 mm MaxRP column. Fractions containing the desired product were lyophilized to afford compound 141 as a yellow powder (8.94 mg, 7.90 μmol, 19%). LC-MS (Method D): t_{R}=1.47 min; MS (m/z) [M+H]⁺ calc. for C₅₂H₅₅N₆O₂₁S 1131.31, found 1131.43.

### Synthetic Example 95

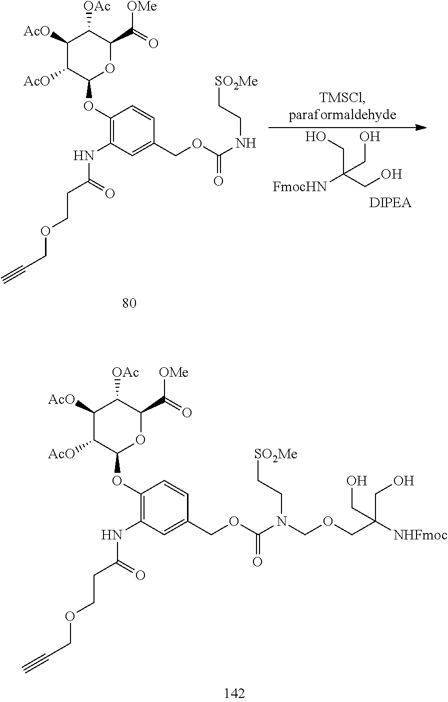

Compound 80 (200 mg, 0.262 mmol) prepared by Example 54 was dissolved in DCM (4 mL). Paraformaldehyde (250 mg) was added followed by TMSCl (2 mL). The reaction was stirred for 20 minutes at which point complete conversion to the activated chloromethyl intermediate observed by quenching a 2 μL aliquot into 98 μL of MeOH to observe the corresponding MeOH adduct by UPLC-MS. The reaction was filtered through a syringe filter, rinse DCM (2 mL), and toluene (2 mL) was added. The solvent was evaporated in vacuo and the final product was placed on high vacuum until ready for use. Fmoc-tris(hydroxymethyl)aminomethane (THAM) was prepared as described in WO 2006/006196. Fmoc-THAM (270 mg, 0.786 mmol) was dissolved in DCM (2 mL) and added directly to the activated intermediate. DIPEA (0.136 mL, 0.786 mmol) was added and the reaction was stirred for 1 hour. Complete conversion was observed by UPLC-MS. The reaction was quenched with MeOH, concentrated in vacuo, and purified by column chromatography using a 50 G KP-Sil column with a 20-100% EtOAc in Hex gradient. Fractions containing the desired product were concentrated in vacuo to afford compound 142 as a colorless solid (242.3 mg, 0.2264 mmol, 86%). LC-MS (Method D): t_{R}=2.04 min; MS (m/z) [M+H]⁺ calc. for C₅₀H₆₀N₃O₂₁S 1070.34, found 1070.42.

### Synthetic Example 96

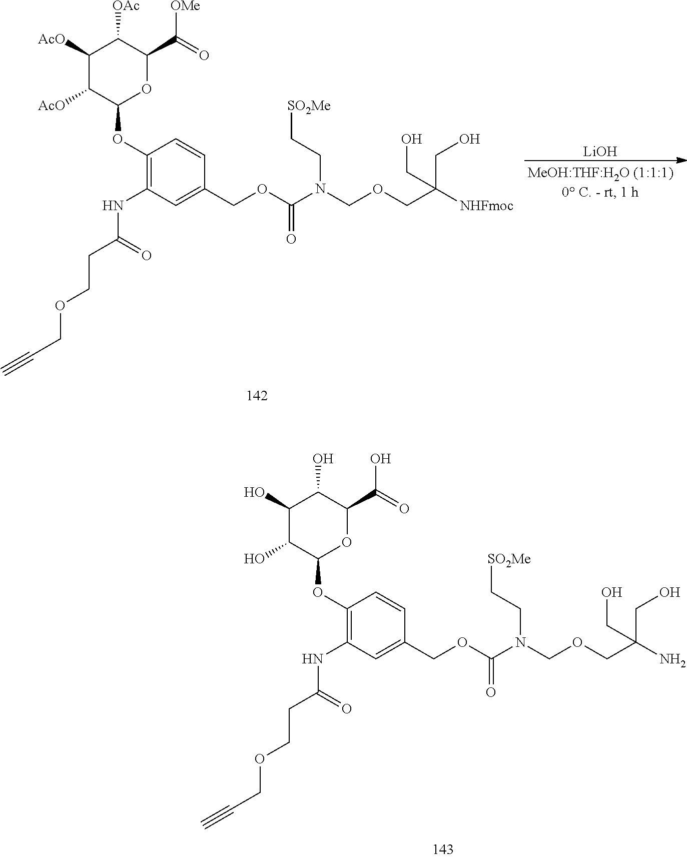

Compound 142 (242.3 mg, 0.2264 mmol) was dissolved in MeOH:THF 1:1 (4 mL) and cooled with an ice/water bath. LiOH (54 mg, 2.6 mmol) was added to the reaction and stirred for 30 minutes. H₂O (2 mL) was added to the reaction, allowed to warm to room temperature and stirred for 30 minutes. Complete conversion to the deprotected product was observed. The reaction was neutralized with AcOH, concentrated in vacuo, and purified by reverse phase flash chromatography using a Biotage C18 Ultra 30 G column eluting with a gradient of 5-20-95% MeCN in H₂O 0.1% Formic Acid. Fractions containing the desired product were concentrated in vacuo to afford compound 143 as a colorless solid (88.3 mg, 0.125 mmol, 55%). LC-MS (Method D): t_{R}=0.60 min; MS (m/z) [M+H]⁺ calc. for C₂₈H₄₂N₃O₁₆S 708.23, found 707.84.

### Synthetic Example 97

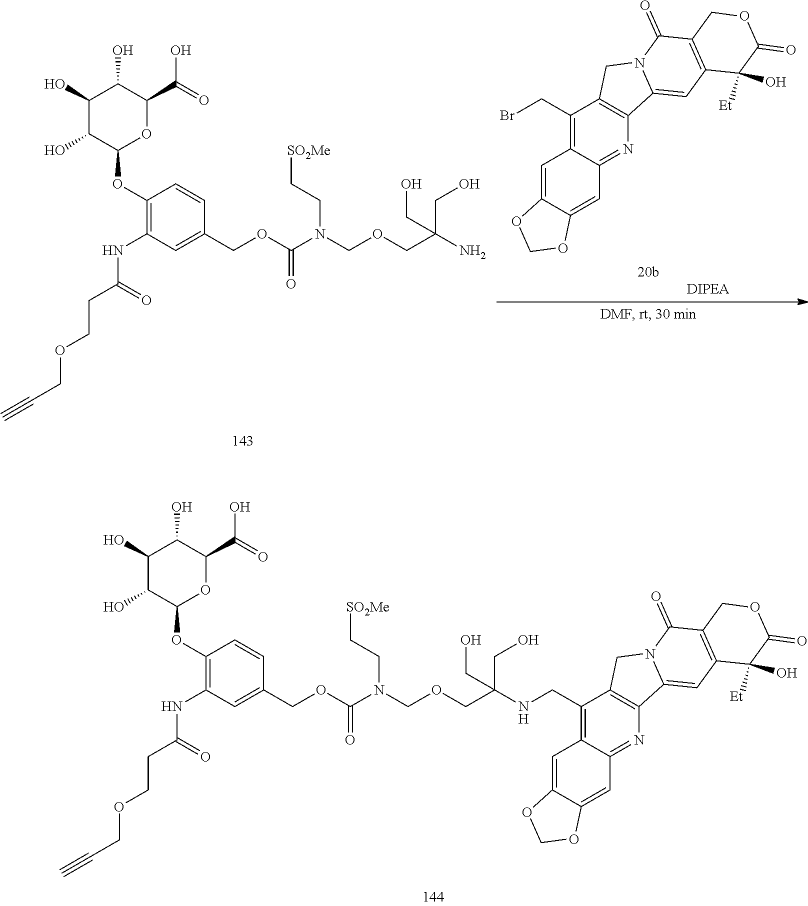 [SMILES: C#CCOCCC(=O)Nc1cc(COC(=O)N(CCS(C)(=O)=O)COCC(N)(CO)CO)ccc1O[C@@H]1O[C@H](C(=O)O)[C@@H](O)[C@H](O)[C@H]1O ; CC[C@@]1(O)C(=O)OCc2c1cc1n(c2=O)Cc2c-1nc1cc3c(cc1c2CBr)OCO3 ; C#CCOCCC(=O)Nc1cc(COC(=O)N(CCS(C)(=O)=O)COCC(CO)(CO)NCc2c3c(nc4cc5c(cc24)OCO5)-c2cc4c(c(=O)n2C3)COC(=O)[C@]4(O)CC)ccc1O[C@@H]1O[C@H](C(=O)O)[C@@H](O)[C@H](O)[C@H]1O]

Compound 143 (88.3 mg, 0.125 mmol) was dissolved in anhydrous DMF (0.5 mL). Compound 20b (30.0 mg, 0.0618 mmol) was added to the reaction followed by DIPEA (0.032 mL, 0.024 mmol). The reaction was stirred for 30 min, at which point complete conversion to product was observed. The reaction quenched with AcOH (0.050 mL), then purified by preparative HPLC using a 21.2×250 mm Max-RP column eluting with a gradient of 5-40-95% MeCN in H₂O 0.1% Formic Acid. Fractions containing the desired product were concentrated in vacuo to afford compound 144 as a yellow solid (1.69 mg, 0.00104 mmol, 1.7%). LC-MS (Method D): t_{R}=1.03 min; MS (m/z) [M+H]⁺ calc. for C₅₀H₅₈N₅O₂₂S 1112.33, found 1112.42.

### Synthetic Example 98

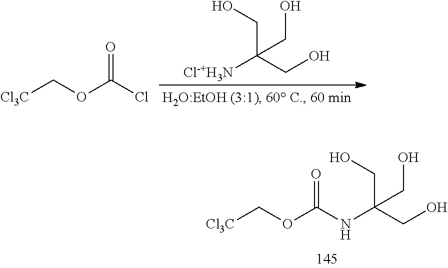

Tris(hydroxymethyl)aminomethane hydrochloride (2.00 g, 4.72 mmol) was dissolved in 3:1 water:ethanol (8 mL). 2,2,2-Trichloroethyl chloroformate (1.32 mL, 4.72 mmol) was added to the reaction mixture. The reaction was stirred at 60° C. for 60 minutes. The reaction was cooled to room temperature, diluted with EtOAc (100 mL), washed with 1M HCl (3×100 mL), washed with sat. NaCl (50 mL), dried MgSO₄, filtered and concentrated in vacuo to afford the compound 145 as a colorless solid (1.64 g, 5.52 mmol, 58%). LC-MS (Method D): t_{R}=1.06 min; MS (m/z) [M+H]⁺ calc. for C₇H₁₃C₁₃NO₅ 295.99, found 296.12.

### Synthetic Example 99

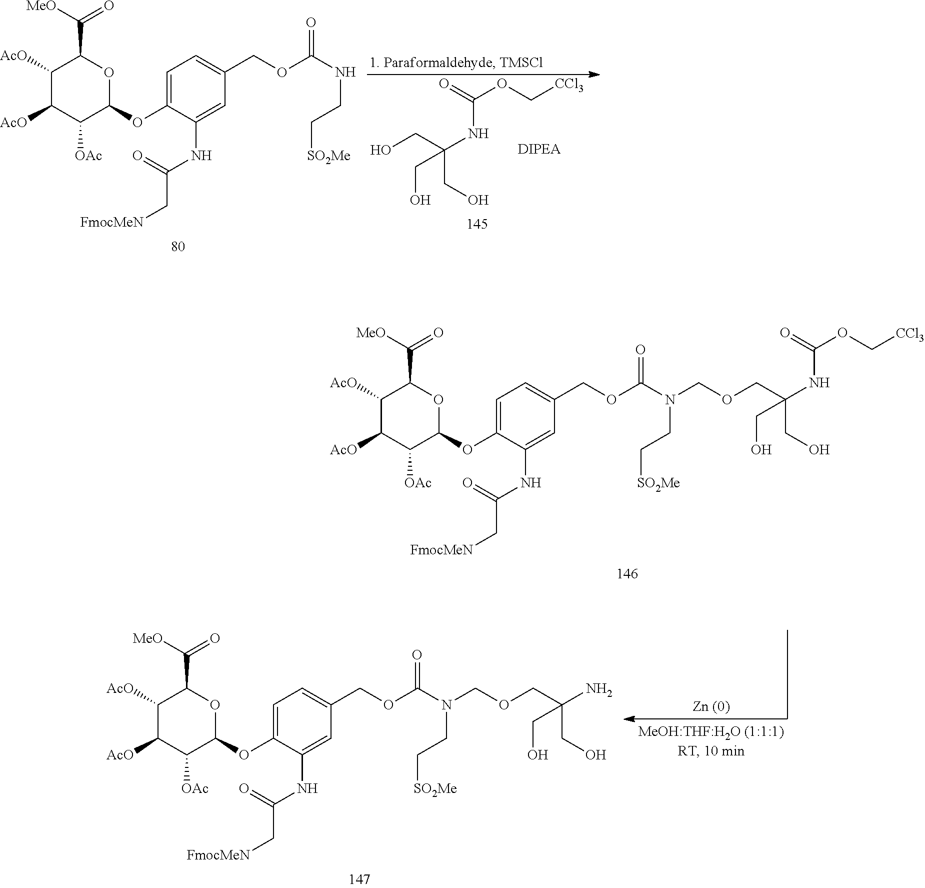

Compound 80 (300 mg, 0.334 mmol) was dissolved in DCM (2 mL). Paraformaldehyde (300 mg, 10.0 mmol) was added followed by TMSCl (1 mL). The reaction was stirred for 10 minutes at which point complete conversion was observed by diluting 2 μL aliquot into 98 μL of MeOH and observing the MeOH adduct by UPLC-MS. The reaction was filtered with a syringe filter, washed with DCM (1 mL), Toluene (2 mL) added to azeotrope final mixture upon concentration. The eluent was concentrated in vacuo to afford a colorless solid. Compound 145 was azeotroped with toluene prior to use. The activated chloromethyl compound was dissolved in anhydrous DCM (1 mL, extra-dry over Mol Sieves). DIPEA (0.23 mL, 1.3 mmol) was added followed by compound 145. The reaction was stirred for 30 minutes at which point complete conversion was observed. The reaction was quenched with MeOH (0.1 mL), concentrated in vacuo to afford compound 146 as a colorless solid which was used in the next step without purification. LC-MS (Method D): t_{R}=2.16 min; MS (m/z) [M+H]⁺ calc. for C₅₀H₆₀C₁₃N₄O₂₂S 1205.25, found 1205.16.

Crude compound 146 (0.334 mmol) was dissolved in 1:1:1 MeOH:THF:AcOH (3 mL). Zinc dust (218 mg, 3.34 mmol) was added and the reaction was stirred for 10 minutes. Complete conversion to the deprotect product was observed by UPLC-MS. The reaction was filtered, and the eluent concentrated. The crude product was purified by preparative HPLC 5-30-50-95% MeCN in H₂O 0.1% formic acid using a 30×250 mm Max-RP column. Fractions containing the desired product were concentrated in vacuo until volume reduced by half, aqueous made basic with sat. NaHCO₃, then extracted with CHCl₃ (3×50 mL), and EtOAc (3×50 mL). The organics were combined and dried with MgSO₄. Filtered and concentrated in vacuo to afford compound 147 as a white solid (103.4 mg, 0.1003 mmol, 30%). LC-MS (Method D): t_{R}=1.65 min; MS (m/z) [M+H]⁺ calc. for C₄₇H₅₉N₄O₂₀S 1031.34, found 1031.42.

### Synthetic Example 99

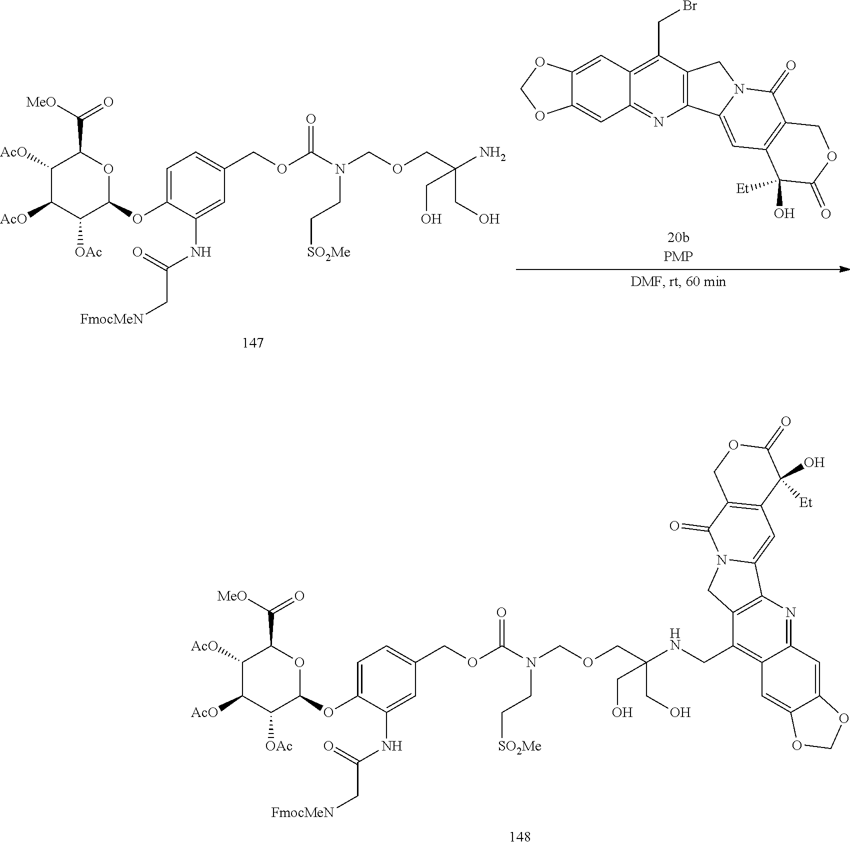

Compound 147 (103.4 mg, 0.1003 mmol) was dissolved in anhydrous DMF (0.5 mL). 1,2,2,6,6-Pentamethylpiperidine (PMP, 0.036 mL, 0.20 mmol) was added followed by compound 20b (97.3 mg, 0.201 mmol). The reaction was stirred for 60 minutes at which point complete conversion was observed by UPLC-MS. The reaction was quenched with AcOH (0.030 mL), then purified by reverse phase flash column chromatography using a 50 G Biotage C18 ultra column eluting with a gradient of 5-60-95% MeCN IN H₂O 0.1% formic acid. Fractions containing the desired product were concentrated to afford compound 148 as an off white solid (18.7 mg, 0.0130 mmol, 13%). LC-MS (Method D): t_{R}=1.79 min; MS (m/z) [M+H]⁺ calc. for C₆₉H₇₅N₆O₂₆S 1435.44, found 1435.27.

### Synthetic Example 100

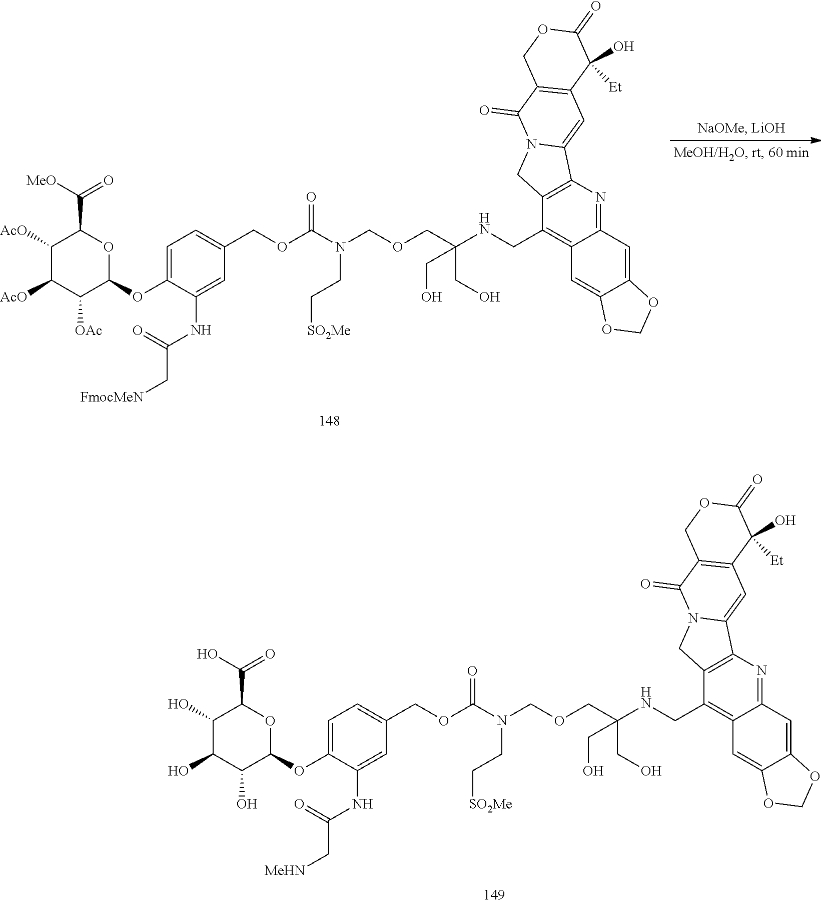

Compound 148 (18.7 mg, 0.0130 mmol) was dissolve in MeOH (1 mL). NaOMe (0.5 M in MeOH, 0.026 mL, 0.013 mmol) was added and the reaction was stirred for 30 minutes. H₂O (1 mL) was added followed by LiOH (1.5 mg, 0.065 mmol) and the reaction was stirred for 30 minutes. Complete conversion to the deprotected product was observe by UPLC-MS. The reaction was neutralized with AcOH, concentrated and purified by preparative HPLC using a 10×250 mm column eluting with a gradient of 5-25-95% MeCN in H₂O 0.1% formic acid. Fractions containing the desired product were concentrated in vacuo to afford compound 149 as a yellow solid (3.9 mg, 0.0036 mmol, 28%). LC-MS (Method D): t_{R}=0.92 min; MS (m/z) [M+H]⁺ calc. for C₄₇H₅₇N₆O₂₁S 1073.33, found 1073.81.

### Synthetic Example 101

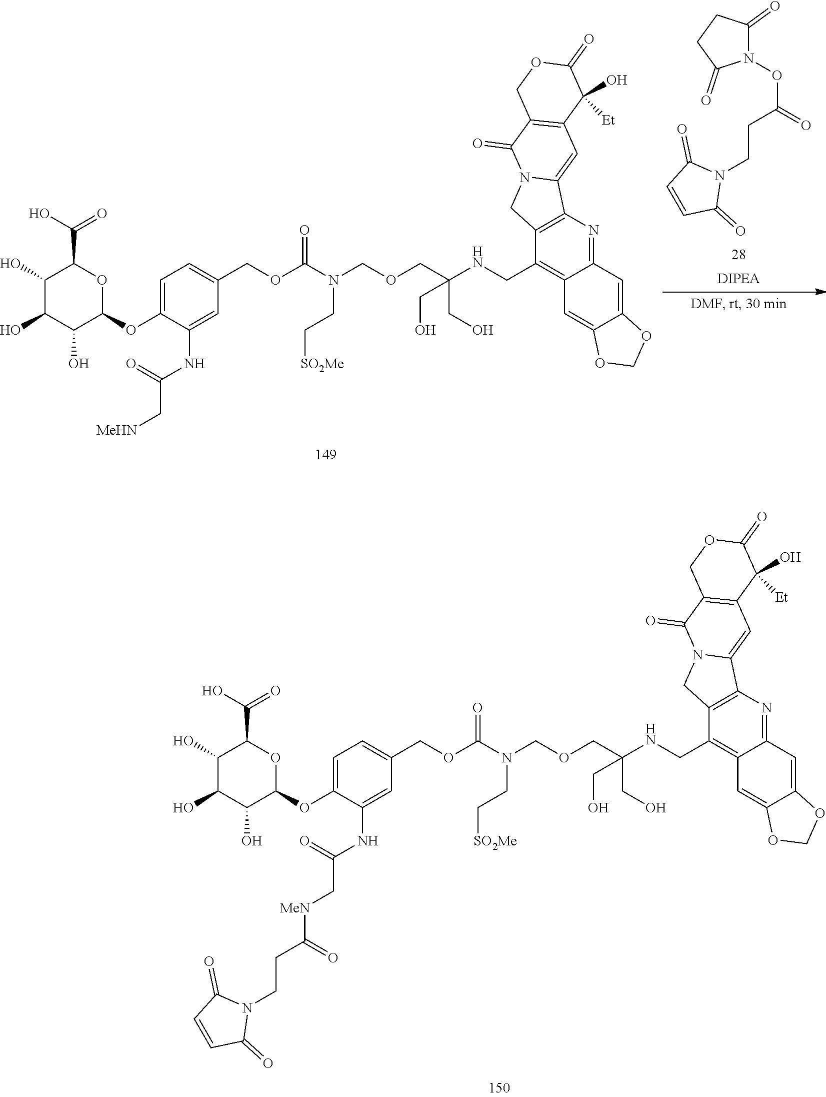 [SMILES: CC[C@@]1(O)C(=O)OCc2c1cc1n(c2=O)Cc2c-1nc1cc3c(cc1c2CNC(CO)(CO)COCN(CCS(C)(=O)=O)C(=O)OCc1ccc(O[C@@H]2O[C@H](C(=O)O)[C@@H](O)[C@H](O)[C@H]2O)c(NC(=O)CNC)c1)OCO3 ; O=C(CCN1C(=O)C=CC1=O)ON1C(=O)CCC1=O ; CC[C@@]1(O)C(=O)OCc2c1cc1n(c2=O)Cc2c-1nc1cc3c(cc1c2CNC(CO)(CO)COCN(CCS(C)(=O)=O)C(=O)OCc1ccc(O[C@@H]2O[C@H](C(=O)O)[C@@H](O)[C@H](O)[C@H]2O)c(NC(=O)CN(C)C(=O)CCN2C(=O)C=CC2=O)c1)OCO3]

Compound 149 (3.9 mg, 0.0036 mmol) was dissolved in anhydrous DMF (0.2 mL). DIPEA (1.3 μL, 0.0073 mmol) was added followed by N-Succinimidyl 3-Maleimidopropionate (1.1 mg, 0.0040 mmol, purchased from TCI America product number S0427). The reaction was stirred for 30 minutes. Complete conversion was observed by UPLC-MS. The reaction was quenched with AcOH (5 μL) and purified by preparative HPLC eluting with 5-30-95% MeCN in H₂O 0.1% formic acid on a 10×250 mm MaxRP column. Fractions containing the desired product were lyophilized to afford compound 126 as a yellow powder (0.23 mg, 0.19 μmol, 5%). Rt=1.06 min CORTECS C₁₈ General Method UPLC. MS (m/z) [M+H]⁺ calc. for C₅₄H₆₂N70₂₄S 1224.36, found 1224.46.

### Biological Example 1. Apparent Affinity of ADC1 DAR8 to CEACAM5 Expressed at the Cell Surface of MKN45 Tumor Cells

For all the biological examples disclosed below, ADC1 is an antibody-drug conjugate that binds to CEACAM5 having the formula of L-(Q-D)ₚ or a salt thereof, wherein L is a Ligand Unit comprising an antibody or antigen binding fragment thereof that binds to CEACAM5, subscript p, which represents the ratio of Drug Unit to antibody (DAR), is an integer ranging from 1 to 16, and wherein -Q-D- has the formula:

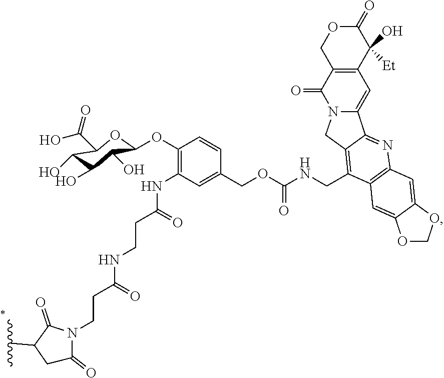

In some embodiments, DAR is 4. In some embodiments, DAR is 8.

In some embodiments, ADC1 is:

L-(Q-D)₈,

wherein L comprises an antibody or antigen binding fragment thereof that binds to CEACAM5 comprising:

a heavy chain that comprises the amino acid sequence set forth in SEQ ID NO:9 and

a light chain that comprises the amino acid sequence set forth in SEQ ID NO:10;

and wherein Q-D is

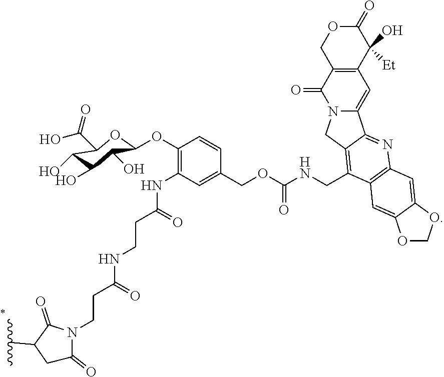

To evaluate the apparent affinity of ADC1 DAR8 to CEACAM5 expressed at the cell surface of MKN45 tumor cells, MKN45 tumor cells were plated at 100,000 cells/well on 96-well plate in 90 μL of assay diluent (DMEM medium supplemented by 5% of fetal calf serum and 1% of bovine serum albumin). Starting at 3 μM up to 11 dilutions 3-fold serial dilutions of ADC1 in assay diluent were performed. 10 μL/well of each dilution were added to 3 wells for 30 min at 4° C. and then washed two times with assay diluent. Cells were resuspended in 100 μL/well of goat anti-human IgG conjugated with Alexa488 (Invitrogen; #A11013) for 30 min at 4° C. and then washed two times with assay diluent. The binding was evaluated after centrifugation and resuspension of cells by adding 100 μl/well of assay diluent and read using Attune Flow Cytometry System (ThermoFisher). Apparent dissociation constant (Apparent K_{D}) was estimated using SPEED software. Results are summarized in Table 1-1.

As shown in Table 1-1, the ADC1 DAR8 binds to CEACAM5 expressed at the cell surface of MKN45 tumor cells with an apparent K_{D} of 15.4±18.1 nM.

TABLE 1-1
In vitro apparent affinity of ADC1 DAR 8 to CEACAM5
expressed at the cell surface of MKN45 cells

| | Apparent Kd (nM) | | | | | | | |
|---|---|---|---|---|---|---|---|---|
| | Exp. | Exp. | Exp. | Exp. | Exp. | Exp. | Exp. | Mean ± |
| | 1 | 2 | 3 | 4 | 5 | 6 | 7 | SD |
| ADC1 | 7.23 | 4.47 | 6.23 | 11.84 | 22.70 | 1.90 | 53.53 | 15.4 ± |
| DAR 8 | | | | | | | | 18.1 |

### Biological Example 2. Binding of ADC1 DAR of 8 to Human FcRn Stably Expressed in HEK293 Cell Line

300,000 HEK293 cells stably over-expressing the protein complex formed by human FcRn and b2-microglobulin were suspended in PBS pH 7.2 or in PBS pH 6.6. 10 mg/ml of ADC1 with a DAR of 8 or of positive control IgG1 isotype antibody were added for 1 hour at 4° C. and then washed two times with 200 mL of PBS pH 7.2 or PBS pH 6.6. Cells were resuspended in 50 μL of goat anti-human IgG conjugated with Alexa488 (Invitrogen; #A11013) diluted in PBS pH 7.2 or PBS pH 6.6 for 1 hour at 4° C. and then washed two times with PBS pH 7.2 or PBS pH 6.6. The binding was evaluated by flow cytometry using MACSQuant Flow Cytometry System (Miltenyi).

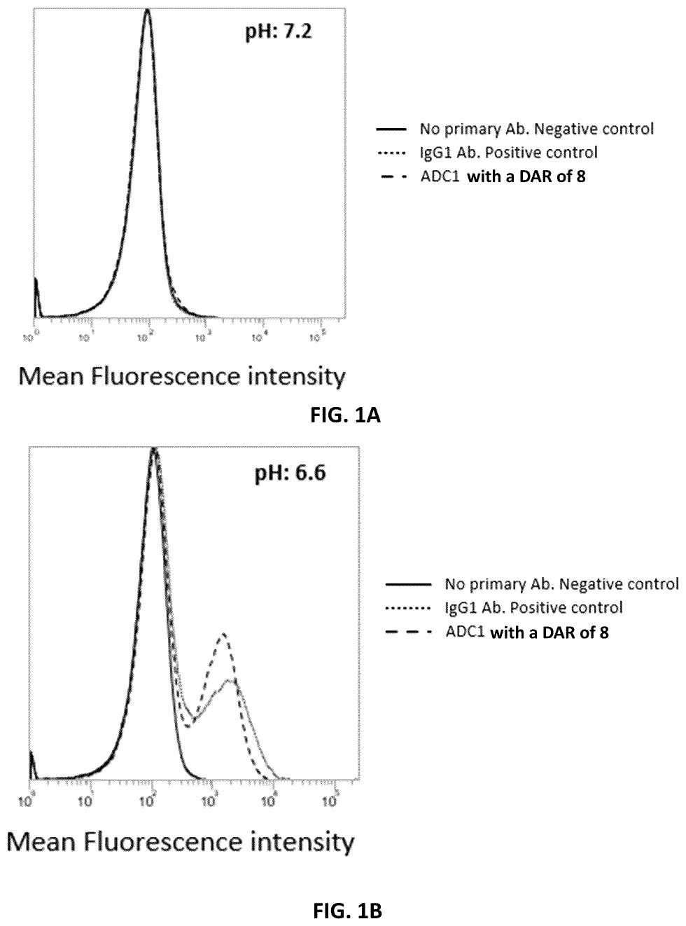

As shown in FIGS. 1A and 1B, as expected there is no binding of IgG1 antibody or of ADC1 DAR 8 to cells expressing FcRN at pH 7.2, while both IgG1 antibody and ADC1 bind to huFcRn when the pH is 6.6.

### Biological Example 3: In Vitro On-Target Cytotoxicity of ADC1 DAR of 8 on a Panel of Tumor Cell Lines

MKN45, LS180 and HCT116 are tumor cell lines with respectively high, moderate and no CEACAM5 expressed at the cell surface. 3,000 cells/well were plated in 90 μL of culture medium and incubated for 4 hours at 37° C. 5% CO₂. Starting at 3 μM up to 11 dilutions, 3-fold serial dilutions of ADC1 with a DAR of 8 in culture medium were performed. 10 μL/well of each dilution were added to 3 wells of the seeded cells. Mix of ADC1 DAR 8 and cells were incubated for 96 hours at 37° C. 5% CO₂. 100 μL/well of Cell Titer-Glo Reagent® (Promega; #G7571) was added to each well. The luminescence was measured using an Envision 2104 plate reader (Perkin-Elmer). ADC1 with a DAR of 8 cytotoxicity and IC₅₀ were estimated using SPEED software.

The activity described below is given by the half maximal effective concentration (EC₅₀), which corresponds to the concentration required to obtain a 50% of the activation. The lower the EC₅₀, the less the concentration of the compound is required to produce 50% of maximum activation and the higher the potency. An EC₅₀<2 nM are considered as demonstrating an activation; EC₅₀≥2 nM and <50 nM are considered as demonstrating a moderate activation. EC₅₀≥100 nM are considered as demonstrating an absence of activation.

As shown in Table 3-1, ADC1 with a DAR of 8 IC₅₀ of cytotoxicity is sub-nanomolar for MKN45 and LS180 tumor cell lines with high CEACAM5 expression and moderate CEACAM5 expression respectively, while it could not be calculated for HCT116, a cell line not expressing CEACAM5, as some cytotoxicity occurred only for highest concentrations of ADC1 DAR 8 assessed.

TABLE 3-1
IC₅₀ of cytotoxicity of ADC1 with a DAR of 8 on tumor
cell lines with different CEACAM5 expression

| | IC₅₀ (nM) | | |
|---|---|---|---|
| | MKN45 | LS180 | HCT116 |
| Agent | High | Moderate | No Expression |
| ADC1 with a DAR | 0.66 | 0.48 | >100 |
| of 8 | 0.65 | 0.39 | >200 |
| | 0.90 | 0.23 | >300 |
| | 0.40 | 0.44 | >300 |
| | 0.51 | 0.46 | >300 |
| Mean ± SD | 0.62 ± 0.19 | 0.40 ± 0.10 | / |

### Biological Example 4: In Vitro Off-Target Cytotoxicity of ADC1 with a DAR of 8 on a Panel of Normal Cells

Normal Human dermal Fibroblast, NHDF, Human Umbilical Vein Endothelial Cell, HUVEC and Normal Human Bronchial Epithelial, NHBE, are normal cells representative of major cell types. None of these cells express CEACAM5. 5,000, 2,000 or 1,500 cells/well for NHDF, HUVEC and NHBE respectively were plated in 90 μL of culture medium and incubated for 4 hours at 37° C./5% CO₂. 3-fold serial dilutions of ADC1 with a DAR of 8 in culture medium were performed, starting at 2 μM up to 11 dilutions. 10 μL/well of each dilution were added to 3 wells of the seeded cells. Mix of ADC1 with a DAR of 8 and cells were incubated for 96 h at 37° C./5% CO₂. 100 μL/well of Cell Titer-Glo Reagent® (Promega; #G7571) was added to each well. The luminescence was measured using an Envision 2104 plate reader (Perkin-Elmer). ADC1 with a DAR of 8, cytotoxicity and IC₅₀ were estimated using SPEED software.

As shown in Table 4-1, the ADC1 with a DAR of 8 IC₅₀ of cytotoxicity could not be calculated for both NHBE and NHDF cells as cytotoxicity started only for highest concentrations of ADC1 DAR 8 assessed. There was no cytotoxicity on HUVEC cells.

TABLE 4-1
In vitro off-target cytotoxicity of
ADC1 with a DAR of 8 on normal cells

| | | IC₅₀ (nM) | | |
|---|---|---|---|---|
| | | HUVEC | NHDF | NHBE |
| ADC1 | Antibody Binding Capacity | 0 | 0 | 0 |
| with a | (Receptor/cells) | | | |
| DAR of | Experiment 1 | No cytotoxicity | >200 | >200 |
| 8 | Experiment 2 | >200 | >200 | >200 |
| | Experiment 3 | >200 | >200 | >200 |

ADC1 DAR8 demonstrated no or very low cytotoxicity towards CEACAM5-negative cells allowing to have an optimized tolerability and therapeutic index.

### Biological Example 5: Bystander Effect of ADC1 with a DAR of 8

Transduction of MKN45 cell by CAS9 and CEACAM5 gRNA lentiviral particles (ThermoFisher, #A32064 & A32042) led to generation of MKN45-KO^{CAM5} no longer expressing CEACAM5 protein as CEACAM5 gene is knocked out. Further transduction by lentiviral particles (Essen Biosciences/Sartorius, #4478) allowing nuclear-restricted red fluorescent labelling of cells led to generation of NLR-MKN45-KO^{CAM5} cell line.

10,000 cells of NLR-MKN45-KO^{CAM5} or 5,000 cells of parental MKN45 cell line plus 5,000 cells of NLR-MKN45-KO^{CAM5} were plated in culture medium in a 96-well plate. 10, 1 or 0.1 nM of ADC1 with a DAR of 8 were each added to 3 wells and number of NLR-MKN45-KO^{CAM5} was counted every 12 hours for 96 hours using an Incucyte® S3 IC50534 (Essen Biosciences/Sartorius).

As shown in Table 5-1, ADC1 with a DAR of 8 does not directly inhibit growth of NLR-MKN45-KO^{CAM5} cells, while in a dose-related effect, active metabolite released by MKN45 killed co-cultured NLR-MKN45-KO^{CAM5} through a bystander effect. In other words, the cytotoxicity of the ADC on the NLR-MKN45-KOCAM5 is mediated by 1/ADC internalization, processing, and active metabolite release by the CEACAM5-expressing MKN-45 cells and 2/by a bystander activity allowing the released active metabolites to diffuse to the neighboring NLR-MKN45-KOCAM5 and their killing.

TABLE 5-1
Evaluation of ADC1 with a DAR of 8 bystander effect

| | Concentration of ADC1 with a DAR of 8 (nM) | | | | | |
|---|---|---|---|---|---|---|
| | | | | Co-culture of MKN45 and | | |
| | NLR-MKN45-KO^{CAM5} alone | | | NLR-MKN45-KO^{CAM5} | | |
| | 0.1 | 1 | 10 | 0.1 | 1 | 10 |
| AUC of NLR-MKN45- | 45886 | 41510 | 41778 | 38700 | 22044 | 18862 |
| KO^{CAM5} | 46104 | 42951 | 41734 | 41736 | 22610 | 20473 |
| | 45529 | 44575 | 41195 | 36935 | 21707 | 19148 |
| Mean | 45839.7 | 43012 | 41569 | 39123.7 | 22120.3 | 19494.3 |
| % of MKN45-KO^{CAM5} | 0% | 6.2% | 9.3% | 14.7% | 51.7% | 57.5% |
| Growth Inhibition | | | | | | |

ADC1 DAR 8 demonstrated a strong bystander effect.

### Biological Example 6: In Vivo Efficacy Evaluation of ADC1 with DAR 8 Against CRC Patient-Derived Xenograft Tumor CR-IGR-0002P (Adenocarcinoma, Primary Colorectal Tumor) Implanted s.c. In Female SCID Mice

ADC1 DAR8 was evaluated at 3 doses against measurable CRC patient-derived xenograft (PDX) CR-IGR-0002P tumors implanted s.c in female SCID mice. Control groups were left untreated. The doses conjugates were given in mg/kg. They were administered at 10, 3 and 1 mg/kg by a single intravenous (IV) bolus injection, on day 23 after tumor implantation.

For the evaluation of anti-tumor activity of conjugates, the animals were weighed and the tumors were measured 2 times weekly by caliper. A dosage producing a 20% weight loss at nadir (mean of group) or 10% or more drug deaths, was considered an excessively toxic dosage. Animal body weights included the tumor weights. Tumor volume was calculated using the formula mass (mm³)=[length (mm)×width (mm)2]/2. The primary efficacy end points are ΔT/ΔC, percent median regression, partial and complete regressions (PR and CR).

Changes in tumor volume for each treated (T) and control (C) are calculated for each tumor by subtracting the tumor volume on the day of randomization (staging day) from the tumor volume on the specified observation day. The median ΔT is calculated for the treated group and the median ΔC is calculated for the control group. Then the ratio ΔT/ΔC is calculated and expressed as a percentage:

ΔT/ΔC=(delta T/delta C)×100

The dose is considered as therapeutically active when ΔT/ΔC is lower than or equal to 40% and very active when ΔT/ΔC is lower than or equal to 10%. If ΔT/ΔC is lower than 0, the dose is considered as highly active, and the percentage of regression is dated (Plowman J, Dykes D J, Hollingshead M, Simpson-Herren L and Alley M C. Human tumor xenograft models in NCI drug development. In: Feibig H H B A, editor. Basel: Karger.; 1999 p 101-125):

% tumor regression: is defined as the % of tumor volume decrease in the treated group at a specified observation day compared to its volume on the day of randomization.

At a specific time point and for each animal, % regression is calculated. The median % regression is then calculated for the group.

$$\% regression ( at t ) = \frac{{volume}_{t 0} - {volume}_{t}}{{volume}_{t 0}} \times 100$$

Partial regression (PR): Regressions are defined as partial if the tumor volume decreases to 50% of the tumor volume at the start of treatment.

Complete regression (CR): Complete regression is achieved when tumor volume=0 mm³ (CR is considered when tumor volume cannot be recorded).

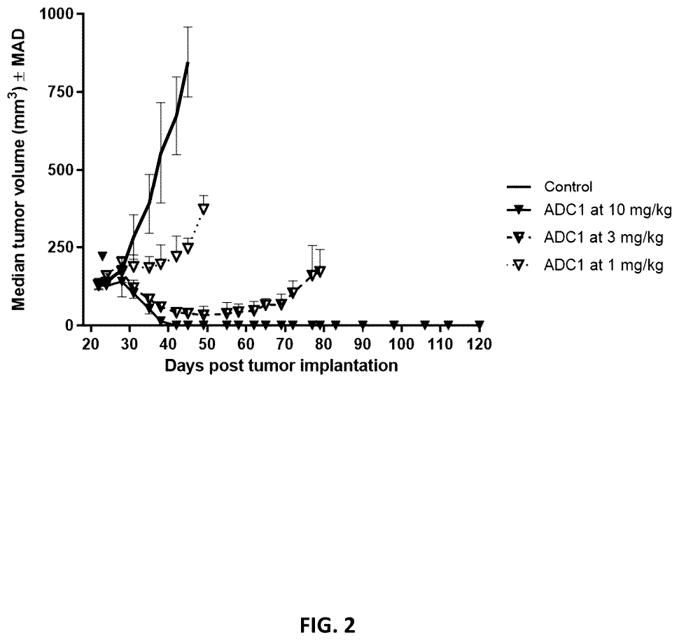

FIG. 2 shows the evaluation of the anti-tumor activity of ADC1 DAR 8 against CRC patient-derived xenograft tumor CR-IGR-0002P in SCID female mice. As shown in FIG. 2 and Table 6-1, using a single administration schedule at 1, 3 and 10 mg/kg, the conjugate tested in this study did not induce toxicity.

The ADC1 DAR 8 was highly active at 10 and 3 mg/kg with a ΔT/ΔC less than 0%, with tumor regression of 100% and 76%, respectively, 6 PR and 6 PR/6, respectively, and 6 and 2 CR/6, respectively. It was very active at 1 mg/kg with a ΔT/ΔC equal to 10%.

Therefore, ADC1 DAR 8 was effective for treating CRC tumors.

TABLE 6-1
Evaluation of the anti-tumor activity of ADC1 DAR 8 against CRC
patient-derived xenograft tumor CR-IGR-0002P in SCID female mice.

| | | | | | Mean body | | | | | |
|---|---|---|---|---|---|---|---|---|---|---|
| | | | | | weight change | Median | Median % | | | |
| | Route/ | | Schedule | Drug | in % per | ΔT/ΔC | of | | | |
| | Dosage | Dosage | in | death | mouse at nadir | in % | regression | Regressions | | |
| Agent | in mL/kg | in mg/kg | days | (Day) | (Day) | (D 49) | (D 49) | PR | CR | Comments |
| ADC1 | IV | 10 | 23 | 0/6 | −3.23 (D 52) | <0 | 100 | 6/6 | 6/6 | Highly |
| DAR 8 | (10 mL/ | | | | | | | | | active |
| | Kg) | 3 | 23 | 0/6 | −2.03 (D 24) | <0 | 76 | 6/6 | 2/6 | Highly |
| | | | | | | | | | | active |
| | | 1 | 23 | 0/6 | −3.50 (D 55) | 10 (D 38) | — | 0/6 | 0/6 | Very |
| | | | | | | | | | | active |
| Control | — | — | — | — | −2.88 (D 55) | — | — | — | — | — |

ΔT/ΔC = ratio of medians of tumor volume changes from baseline between treated and control groups;
PR = Partial regression;
CR = Complete regression

### Biological Example 7: In Vivo Efficacy Evaluation of ADC1 DAR 8 Against CRC Patient-Derived Xenograft Tumor, CR-IGR-0007P (Adenocarcinoma, Primary Colorectal Tumor) Implanted s.c. In Female SCID Mice

ADC1 DAR 8 was evaluated at 3 doses against measurable CRC PDX CR-IGR-0007P tumors implanted s.c in female SCID mice. Control groups were left untreated. The doses conjugates were given in mg/kg. They were administered at 10, 3 and 1 mg/kg by a single intravenous (IV) bolus injection, on day 21 after tumor implantation.

Toxicity and efficacy evaluation were performed as reported in Biological Example 6.

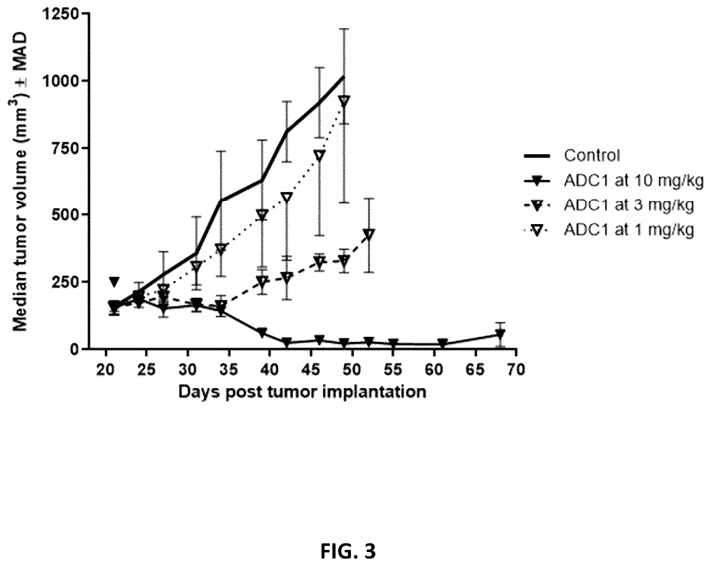

As shown in FIG. 3 and Table 7-1, using a single administration schedule at 1, 3 and 10 mg/kg, the conjugate tested in this study did not induce toxicity.

The following examples, including the experiments conducted and the results achieved, are provided for illustrative purposes only and are not to be construed as limiting the scope of the appended claims.

ADC1 DAR 8 was highly active at 10 mg/kg with a ΔT/ΔC less than 0%, with tumor regression of 93%, 6 PR/6 and 3 CR/6. It was very active at 3 mg/kg with a ΔT/ΔC equal to 3% and 1 PR/6 and inactive at 1 mg/kg with a ΔT/ΔC equal to 86%

Therefore, ADC1 DAR 8 was effective for treating CRC tumors.

TABLE 7-1
Evaluation of the anti-tumor activity of ADC1 DAR 8 against CRC
patient-derived xenograft tumor, CR-IGR-0007P in SCID female mice

| | | | | | Mean body | | | | | |
|---|---|---|---|---|---|---|---|---|---|---|
| | Route/ | | | | weight change | Median | Median % | | | |
| | Dosage | Dosage | Schedule | Drug | in % per | ΔT/ΔC | of | | | |
| | in | in | in | death | mouse at nadir | in % | regression | Regressions | | |
| Agent | mL/kg | mg/kg | days | (Day) | (Day) | (D 49) | (Day) | PR | CR | Comments |
| ADC1 | IV | 10 | 21 | 0/6 | −2.43 (D 24) | <0 | 93 (D 61) | 6/6 | 3/6 | Highly |
| DAR 8 | (10 mL/ | | | | | | | | | active |
| | Kg) | 3 | 21 | 0/6 | +0.02 (D 27) | 3 (D 34) | — | 1/6 | 0/6 | Very |
| | | | | | | | | | | active |
| | | 1 | 21 | 0/6 | +1.68 (D 42) | 86 | | 0/6 | 0/6 | Inactive |
| Control | — | — | — | — | −1.50 (D 49) | — | — | — | — | — |

ΔT/ΔC = ratio of medians of tumor volume changes from baseline between treated and control groups;
PR = Partial regression;
CR = Complete regression

### Biological Example 8: In Vivo Efficacy Evaluation of ADC1 DAR 8 Against CRC Patient-Derived Xenograft Tumor, CR-IGR-0048M (Adenocarcinoma, Metastatic Colorectal Tumor) Implanted s.c. In Female SCID Mice

ADC1 with DAR 8 was evaluated at 2 doses against measurable CRC PDX CR-IGR-0048M tumors implanted s.c in female SCID mice. Control groups were left untreated. The doses conjugates were given in mg/kg. They were administered at 10 and 3 mg/kg by a single intravenous (IV) bolus injection, on day 24 after tumor implantation.

Toxicity and efficacy evaluation were performed as reported in Biological Example 6.

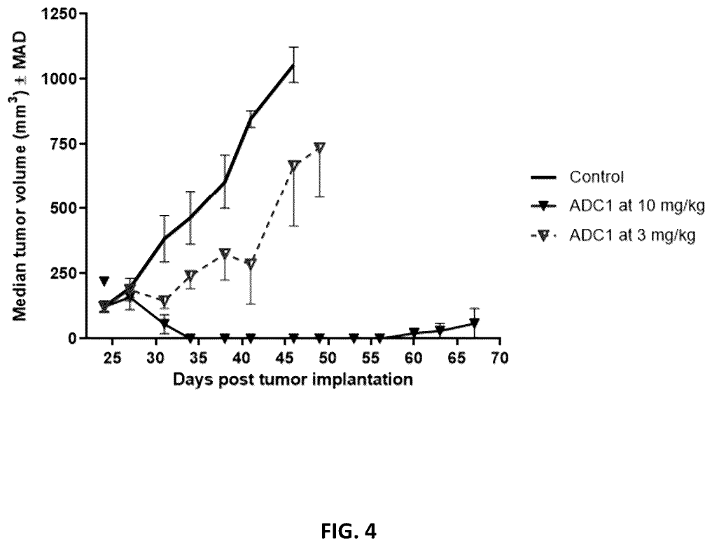

As shown in FIG. 4 and Table 8-1, using a single administration schedule at 3 and 10 mg/kg, the conjugate tested in this study did not induce toxicity.

The ADC1 with DAR 8 was highly active at 10 mg/kg with a ΔT/ΔC less than 0%, with tumor regression of 100%, 6 PR/6 and 4 CR/6. It was marginally active at 3 mg/kg with a ΔT/ΔC equal to 36% and 1 PR/6.

Therefore, ADC1 DAR 8 was effective for treating CRC tumors.

TABLE 8-1
Evaluation of the anti-tumor activity of ADC1 DAR 8 against human CRC
patient-derived xenograft tumor, CR-IGR-0048M in SCID female mice

| | | | | | Mean body | | | | | |
|---|---|---|---|---|---|---|---|---|---|---|
| | Route/ | | | | weight change | Median | Median % | | | |
| | Dosage | | Schedule | Drug | in % per | ΔT/ΔC | of | | | |
| | in | Dosage | in | death | mouse at nadir | in % | regression | Regressions | | |
| Agent | mL/kg | in mg/kg | days | (Day) | (Day) | (D 46) | (Day) | PR | CR | Comments |
| ADC1 | IV | 10 | 24 | 0/6 | −1.07 (D 27) | <0 | 100 (D 46) | 6/6 | 4/6 | Highly |
| DAR 8 | (10 mL/ | | | | | | | | | active |
| | Kg) | 3 | 24 | 0/6 | −3.27 (D 27) | 36 (D 38) | — | 1/6 | 0/6 | Marginally |
| | | | | | | | | | | active |
| Control | — | — | — | — | −0.58 (D 27) | — | — | — | — | — |

ΔT/ΔC = ratio of medians of tumor volume changes from baseline between treated and control groups;
PR = Partial regression;
CR = Complete regression

### Biological Example 9: In Vivo Efficacy Evaluation of ADC1 with DAR 8 Against CRC Patient-Derived Xenograft Tumor, CR-IC-0016M (Adenocarcinoma, Metastastic Colorectal Tumor) Implanted s.c. In Female SCID Mice

The ADC1 with DAR 8 was evaluated at 3 doses against measurable colon PDX CR-IC-0016M tumors implanted s.c in female SCID mice. Control groups were left untreated. The doses conjugates were given in mg/kg. They were administered at 10, 3 and 1 mg/kg by a single intravenous (IV) bolus injection, on day 21 after tumor implantation.

Toxicity and efficacy evaluation were performed as reported in Biological Example 6.

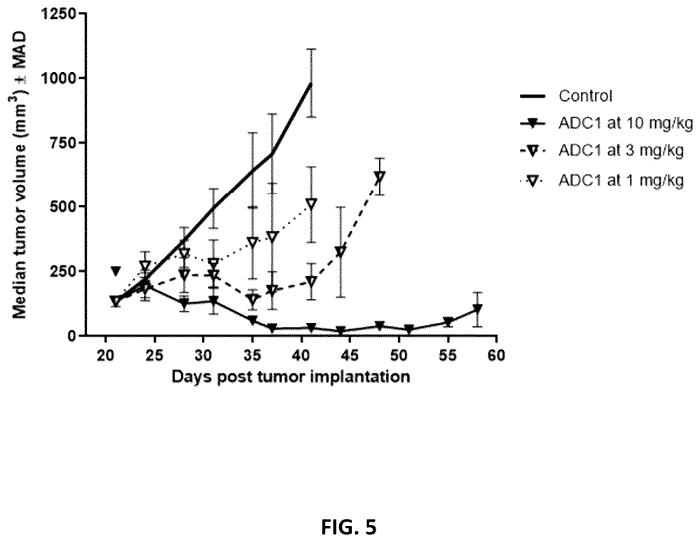

As shown in FIG. 5 and Table 9-1, using a single administration schedule at 1, 3 and 10 mg/kg, the conjugate tested in this study did not induce toxicity.

The ADC1 DAR 8 was highly active at 10 mg/kg with a ΔT/ΔC less than 0%, with tumor regression of 86%, 8 PR/8 and 2 CR/8. It was very active at 3 mg/kg with a ΔT/ΔC equal to 8% and 1 PR/8, and inactive at 1 mg/kg with a ΔT/ΔC equal to 45%.

Therefore, ADC1 with DAR 8 was effective for treating CRC tumors.

TABLE 9-1
Evaluation of the anti-tumor activity of ADC1 with DAR 8 against CRC
patient-derived xenograft tumor, CR-IC-0016M in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 41) | (Day) | PR | CR | Comments |
| ADC1 | IV (10 | 10 | 21 | 0/8 | −1.95 (D 24) | <0 | 86 (D 44) | 8/8 | 2/8 | Highly active |
| DAR 8 | mL/Kg) | 3 | 21 | 0/8 | −3.59 (D 48) | 8 | — | 1/8 | 0/8 | Very active |
| | | 1 | 21 | 0/8 | −2.08 (D 41) | 45 | — | 0/8 | 0/8 | Inactive |
| Control | — | — | — | — | −5.23 (D 35) | — | — | — | — | — |

ΔT/ΔC = ratio of medians of tumor volume changes from baseline between treated and control groups;
PR = Partial regression;
CR = Complete regression

### Biological Example 10: In Vivo Efficacy Evaluation of ADC1 with DAR 8 Against Lung Patient-Derived Xenograft Tumor, LUN-NIC-0014 (Nsq-NSCLC, Adenocarcinoma) Implanted s.c. In Female SCID Mice

The ADC1 with DAR 8 was evaluated at 3 doses against measurable lung PDX LUN-NIC-0014 tumors implanted s.c in female SCID mice. Control groups were left untreated. The doses conjugates were given in mg/kg. They were administered at 10, 3 and 1 mg/kg by a single intravenous (IV) bolus injection, on day 20 after tumor implantation.

Toxicity and efficacy evaluation were performed as reported in Biological Example 6.

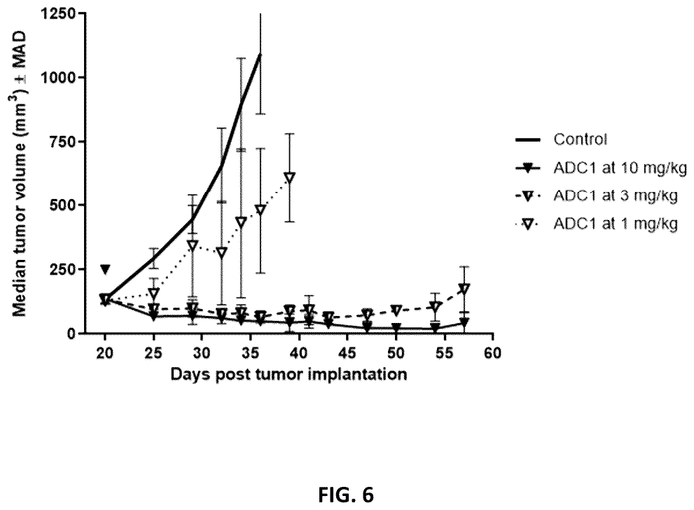

As shown in FIG. 6 and Table 10-1, using a single administration schedule at 1, 3 and 10 mg/kg, the conjugate tested in this study did not induce toxicity.

The ADC1 with DAR 8 was highly active at 10 and 3 mg/kg with a ΔT/ΔC less than 0%, with tumor regression of 83% and 58%, respectively; 5 PR and 4 PR/6, respectively, and 3 CR/6 for the highest dose. It was marginally active at 1 mg/kg with a ΔT/ΔC equal to 35%.

Therefore, ADC1 with DAR 8 was effective for treating lung tumors.

TABLE 10-1
Evaluation of the anti-tumor activity of ADC1 with DAR 8 against lung
patient-derived xenograft tumor, LUN-NIC-0014 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 36) | (Day) | PR | CR | Comments |
| ADC1 | IV (10 | 10 | 20 | 0/6 | −1.23 (D 22) | <0 | 83 (D 47) | 5/6 | 3/6 | Highly active |
| DAR 8 | mL/Kg) | 3 | 20 | 0/6 | −3.43 (D 39) | <0 | 58 (D 43) | 4/6 | 0/6 | Highly active |
| | | 1 | 20 | 0/6 | −5.25 (D 32) | 35 (D 32) | — | 0/6 | 0/6 | Marginally active |
| Control | — | — | — | — | −3.85 (D 32) | — | — | — | — | — |

ΔT/ΔC = ratio of medians of tumor volume changes from baseline between treated and control groups;
PR = Partial regression;
CR = Complete regression

### Biological Example 11: In Vivo Efficacy Evaluation of ADC1 with DAR 8 Against Lung Patient-Derived Xenograft Tumor, LUN-NIC-0084 (Nsq-NSCLC, Adenocarcinoma) Implanted s.c. In Female SCID Mice

The ADC1 with DAR 8 was evaluated at 3 doses against measurable lung PDX LUN-NIC-0084 tumors implanted s.c in female SCID mice. Control groups were left untreated. The doses conjugates were given in mg/kg. They were administered at 10, 3 and 1 mg/kg by a single intravenous (IV) bolus injection, on day 38 after tumor implantation.

Toxicity and efficacy evaluation were performed as reported in Biological Example 6.

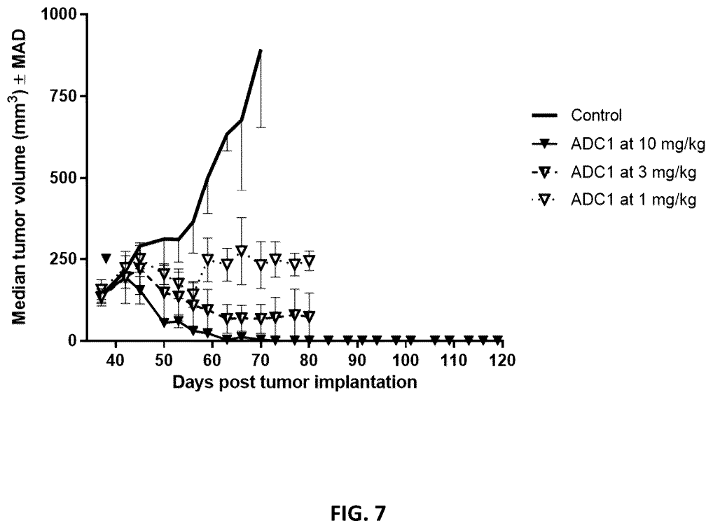

As shown in FIG. 7 and Table 11-1, using a single administration schedule at 1, 3 and 10 mg/kg, the conjugate tested in this study did not induce toxicity.

The ADC1 with DAR 8 was highly active at 10 and 3 mg/kg with a ΔT/ΔC less than 0%, with tumor regression of 100% and 54%, respectively, 6 PR and 3 PR/6, respectively, and 5 CR and 2 CR/6%, respectively. It was active at 1 mg/kg with a ΔT/ΔC equal to 11%.

Therefore, ADC1 with DAR 8 was effective for treating lung tumors.

TABLE 11-1
Evaluation of the anti-tumor activity of ADC1 with DAR 8 against lung
patient-derived xenograft tumor, LUN-NIC-0084 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 70) | (Day) | PR | CR | Comments |
| ADC1 | IV (10 | 10 | 38 | 0/6 | −1.83 (D 46) | <0 | 100 (D 70) | 6/6 | 5/6 | Highly active |
| DAR 8 | mL/Kg) | 3 | 38 | 0/6 | −2.00 (D 42) | <0 | 54 (D 63) | 3/6 | 2/6 | Highly active |
| | | 1 | 38 | 0/6 | +1.53 (D 38) | 11 | — | 0/6 | 0/6 | Active |
| Control | — | — | — | — | −0.20 (D 70) | — | — | — | — | — |

ΔT/ΔC = ratio of medians of tumor volume changes from baseline between treated and control groups;
PR = Partial regression;
CR = Complete regression

### Biological Example 12: In Vivo Efficacy Evaluation of ADC1 with DAR 8 Against Lung Patient-Derived Xenograft Tumor, LUN-NIC-0004 (Nsq-NSCLC, Adenocarcinoma) Implanted s.c. In Female SCID Mice

The ADC1 with DAR 8 was evaluated at 3 doses against measurable lung PDX LUN-NIC-0004 tumors implanted s.c in female SCID mice. Control groups were left untreated. The doses conjugates were given in mg/kg. They were administered at 10, 3 and 1 mg/kg by a single intravenous (IV) bolus injection, on day 58 after tumor implantation.

Toxicity and efficacy evaluation were performed as reported in Biological Example 6.

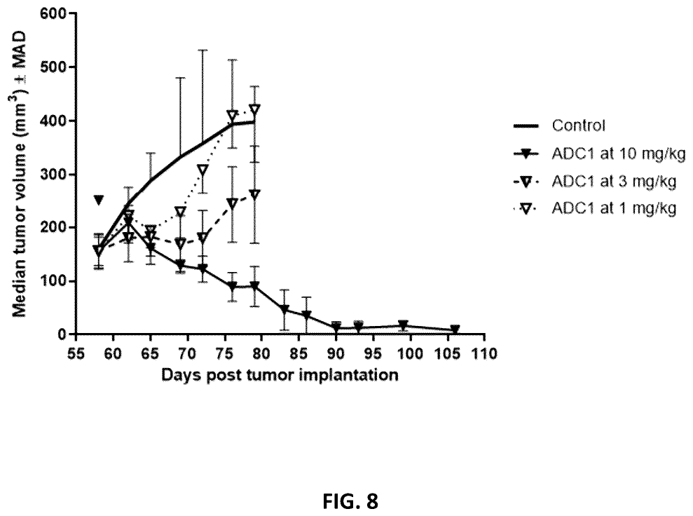

As shown in FIG. 8 and Table 12-1, using a single administration schedule at 1, 3 and 10 mg/kg, the conjugate tested in this study did not induce toxicity.

The ADC1 with DAR 8 was highly active at 10 mg/kg with a ΔT/ΔC less than 0%, with tumor regression of 96%, 6 PR/6, and 3 CR/6%. It was active at 1 mg/kg with a ΔT/ΔC equal to 31% and inactive at 1 mg/kg with a ΔT/ΔC equal to 107%.

Therefore, ADC1 with DAR 8 was effective for treating lung tumors.

TABLE 12-1
Evaluation of the anti-tumor activity of ADC1 DAR 8 against lung patient-
derived xenograft tumor, LUN-NIC-0004 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 79) | (Day) | PR | CR | Comments |
| ADC1 | IV (10 | 10 | 58 | 0/6 | −0.07 (D 62) | <0 | 96 (D 106) | 6/6 | 3/6 | Highly active |
| DAR 8 | mL/Kg) | 3 | 58 | 0/6 | +1.88 (D 62) | 31 | — | 0/6 | 0/6 | Active |
| | | 1 | 58 | 0/6 | +1.57 (D 79) | 107 | — | 0/6 | 0/6 | Inactive |
| Control | — | — | — | — | −5.90 (D 79) | — | — | — | — | — |

ΔT/ΔC = ratio of medians of tumor volume changes from baseline between treated and control groups;
PR = Partial regression;
CR = Complete regression

Biological Example 13: In vivo efficacy evaluation of ADC1 with DAR 8 against lung patient-derived xenograft tumor, LUN-NIC-0008 (Sq-NSCLC) implanted s.c. in female SCID mice

The ADC1 with DAR 8 was evaluated at 3 doses against measurable lung PDX LUN-NIC-0008 tumors implanted s.c in female SCID mice. Control groups were left untreated. The doses conjugates were given in mg/kg. They were administered at 10, 3 and 1 mg/kg by a single intravenous (IV) bolus injection, on day 28 after tumor implantation.

Toxicity and efficacy evaluation were performed as reported in Biological Example 6.

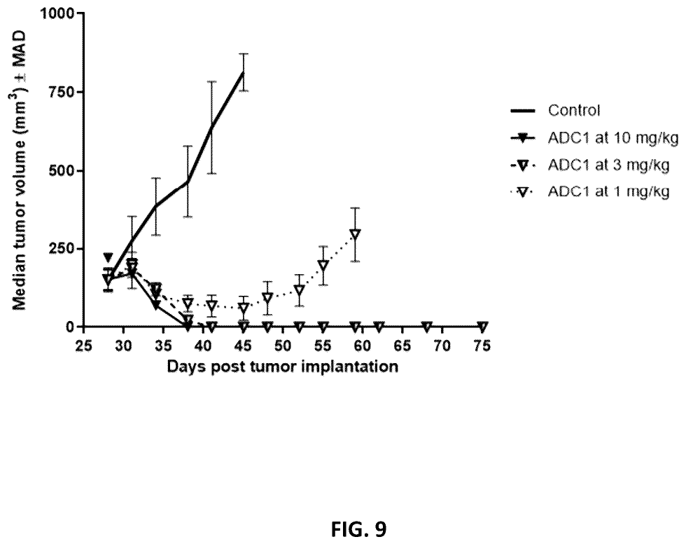

As shown in FIG. 9 and Table 13-1, using a single administration schedule at 1, 3 and 10 mg/kg, the conjugate tested in this study did not induce toxicity.

The ADC1 with DAR 8 was highly active at 10, 3 and 1 mg/kg with a ΔT/ΔC less than 0%, with tumor regression of 100%, 100% and 66%, respectively; 6 PR, 6 PR and 4 PR/6, respectively; and 6 CR, 6 CR and no CR/6%, respectively.

Therefore, ADC1 with DAR 8 was effective for treating lung tumors.

TABLE 13-1
Evaluation of the anti-tumor activity of ADC1 DAR 8 against lung
patient-derived xenograft tumor, LUN-NIC-0008 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 45) | (D 45) | PR | CR | Comments |
| ADC1 | IV (10 | 10 | 28 | 0/6 | −1.65 (D 31) | <0 | 100 | 6/6 | 6/6 | Highly active |
| DAR 8 | mL/Kg) | 3 | 28 | 0/6 | −5.10 (D 31) | <0 | 100 | 6/6 | 6/6 | Highly active |
| | | 1 | 28 | 0/6 | −1.78 (D 31) | <0 | 68 | 4/6 | 0/6 | Highly active |
| Control | — | — | — | — | −7.36 (D 44) | — | — | — | — | — |

ΔT/ΔC = ratio of medians of tumor volume changes from baseline between treated and control groups;
PR = Partial regression;
CR = Complete regression

### Biological Example 14: In Vivo Efficacy Evaluation of ADC1 with DAR 8 Against Gastric Patient-Derived Xenograft Tumor, STO-IND-0006 (Adenocarcinoma) Implanted s.c. In Female SCID Mice

The ADC1 with DAR 8 was evaluated at 3 doses against measurable gastric PDX STO-IND-0006 tumors implanted s.c in female SCID mice. Control groups were left untreated. The doses conjugates were given in mg/kg. They were administered at 10, 3 and 1 mg/kg by a single intravenous (IV) bolus injection, on day 16 after tumor implantation.

Toxicity and efficacy evaluation were performed as reported in Biological Example 6.

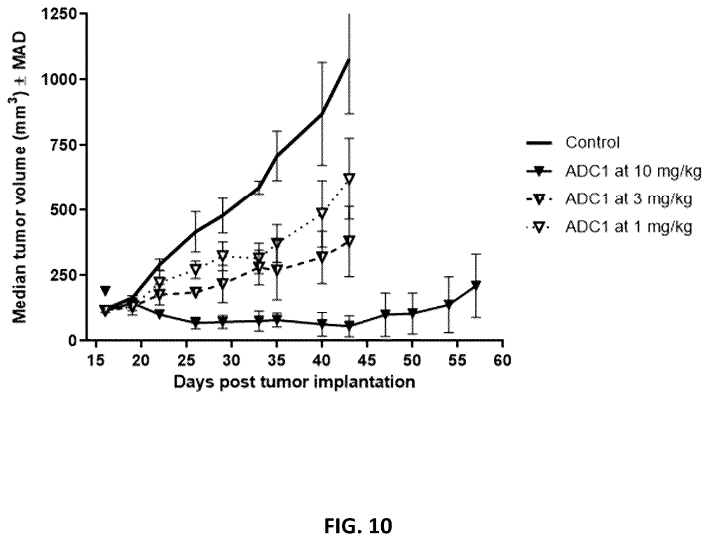

The STO-IND-0006 PDX is an aggressive tumor that can be cachexic and induces body weight loss and requires premature ethical euthanasia. As shown in FIG. 10 and Table 14-1, using a single administration schedule at 1, 3 and 10 mg/kg, the conjugate tested in this study did not induce additional toxicity.

The ADC1 with DAR 8 was highly active at 10 mg/kg with a ΔT/ΔC less than 0%, with tumor regression of 46%, and 4 PR/6. It was active at 3 mg/kg with a ΔT/ΔC equal to 28%, and inactive at 1 mg/kg with a ΔT/ΔC equal to 51%.

Therefore ADC1 with DAR 8 was effective for treating gastric tumors.

TABLE 14-1
Evaluation of the anti-tumor activity of ADC1 with DAR 8 against gastric
patient-derived xenograft tumor, STO-IND-0006 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 43) | (Day) | PR | CR | Comments |
| ADC1 | IV (10 | 10 | 16 | 0/6 | −9.52 (D 41) | <0 | 46 (D 35) | 4/6 | 0/6 | Highly active |
| DAR 8 | mL/Kg) | 3 | 16 | 0/6 | −11.58 (D 37) | 28 | — | 0/6 | 0/6 | Active |
| | | 1 | 16 | 0/6 | −13.05 (D 40) | 51 | — | 0/6 | 0/6 | Inactive |
| Control | — | — | — | — | −0.79 (D 41) | — | — | — | — | — |

ΔT/ΔC = ratio of medians of tumor volume changes from baseline between treated and control groups;
PR = Partial regression;
CR = Complete regression

### Biological Example 15: In Vivo Efficacy Evaluation of ADC1 with DAR 8 Against Gastric Patient-Derived Xenograft Tumor, SA-STO-0014 (Adenocarcinoma) Implanted s.c. In Female SCID Mice

The ADC1 with DAR 8 was evaluated at 3 doses against measurable gastric PDX SA-STO-0014 tumors implanted s.c in female SCID mice. Control groups were left untreated. The doses conjugates were given in mg/kg. They were administered at 10, 3 and 1 mg/kg by a single intravenous (IV) bolus injection, on day 20 after tumor implantation.

Toxicity and efficacy evaluation were performed as reported in Biological Example 6.

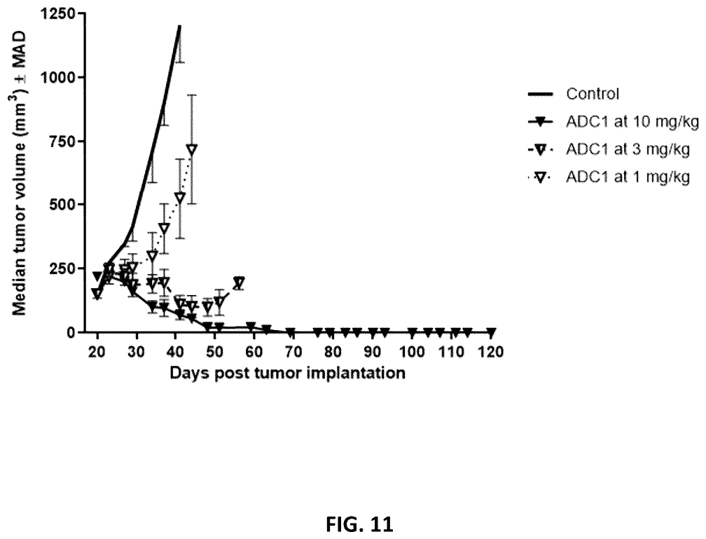

As shown in FIG. 11 and Table 15-1, using a single administration schedule at 1, 3 and 10 mg/kg, the conjugate tested in this study did not induce toxicity.

The ADC1 with DAR 8 was highly active at 10 and 3 mg/kg with a ΔT/ΔC less than 0%, with tumor regression of 100% and 34%, respectively, 6 PR and 2 PR/6, respectively and 6 CR/6 for the highest dose. It was marginally active at 1 mg/kg with a ΔT/ΔC equal to 37%.

Therefore, ADC1 with DAR 8 was effective for treating gastric tumors.

TABLE 15-1
Evaluation of the anti-tumor activity of ADC1 DAR 8 against gastric
patient-derived xenograft tumor, SA-STO-0014 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 41) | (Day) | PR | CR | Comments |
| ADC1 | IV (10 | 10 | 20 | 0/6 | +1.13 (D 23) | <0 | 100 (D 69) | 6/6 | 6/6 | Highly active |
| DAR 8 | mL/Kg) | 3 | 20 | 0/6 | +0.73 (D 27) | <0 | 34 (D 48) | 2/6 | 0/6 | Highly active |
| | | 1 | 20 | 0/6 | +0.98 (D 27) | 37 | — | 0/6 | 0/6 | Marginally active |
| Control | — | — | — | — | +0.33 (D 23) | — | — | — | — | — |

ΔT/ΔC = ratio of medians of tumor volume changes from baseline between treated and control groups;
PR = Partial regression;
CR = Complete regression

### Biological Example 16: In Vivo Efficacy Evaluation of ADC1 with DAR 8 Against Gastric Patient-Derived Xenograft Tumor, STO-IND-0007 (Adenocarcinoma) Implanted s.c. In Female SCID Mice

The ADC1 with DAR 8 was evaluated at 3 doses against measurable gastric PDX STO-IND-0007 tumors implanted s.c in female SCID mice. Control groups were left untreated. The doses conjugates were given in mg/kg. They were administered at 10, 3 and 1 mg/kg by a single intravenous (IV) bolus injection, on day 43 after tumor implantation.

Toxicity and efficacy evaluation were performed as reported in Biological Example 6.

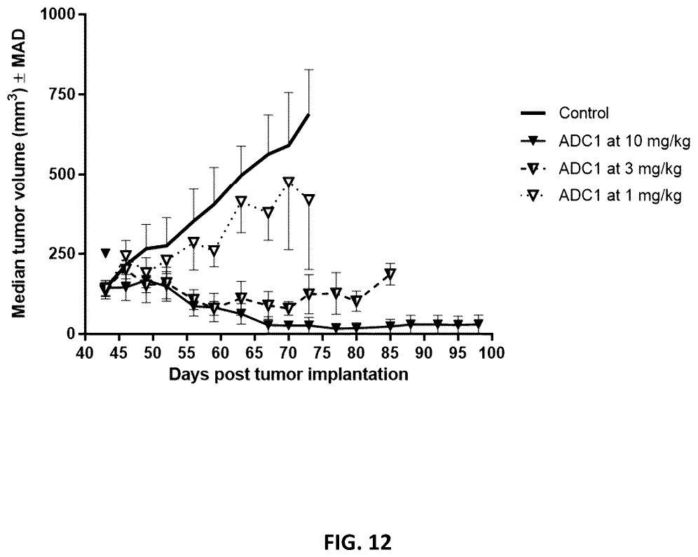

As shown in FIG. 12 and Table 16-1, using a single administration schedule at 1, 3 and 10 mg/kg, the conjugate tested in this study did not induce toxicity.

The ADC1 with DAR 8 was highly active at 10 and 3 mg/kg with a ΔT/ΔC less than 0%, with tumor regression of 91% and 40%, respectively, 6 PR and 3 PR/6, respectively and 3 CR/6 for the highest dose. It was inactive at 1 mg/kg with a ΔT/ΔC equal to 55%.

Therefore, ADC1 with DAR 8 was effective for treating gastric tumors.

TABLE 16-1
Evaluation of the anti-tumor activity of ADC1 with DAR 8 against gastric
patient-derived xenograft tumor, STO-IND-0007 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 73) | (Day) | PR | CR | Comments |
| ADC1 | IV (10 | 10 | 43 | 0/6 | −4.03 (D 46) | <0 | 91 (D 77) | 6/6 | 3/6 | Highly active |
| DAR 8 | mL/Kg) | 3 | 43 | 0/6 | −5.70 (D 46) | <0 | 40 (D 70) | 3/6 | 0/6 | Highly active |
| | | 1 | 43 | 0/6 | −1.85 (D 46) | 55 | — | 0/6 | 0/6 | Inactive |
| Control | — | — | — | — | −4.07 (D 67) | — | — | — | — | — |

ΔT/ΔC = ratio of medians of tumor volume changes from baseline between treated and control groups;
PR = Partial regression;
CR = Complete regression

### Biological Example 17: In Vivo Efficacy Evaluation of ADC1 with DAR 8 in a Panel of 16 CRC Patient-Derived Xenograft Models Under Single Mouse Trial Format

The Single Mouse Trial (SMT) consists in use of one animal per PDX model per treatment arm to evaluate the efficacy of antitumor agents on a larger screening scope, more representative of heterogeneous patient response.

The ADC1 with DAR 8 was evaluated at 10 mg/kg in a panel of 16 CRC PDX models. The dose was given in mg/kg. It was administered by a single intravenous (IV) bolus injection, on measurable CRC PDX tumors implanted s.c in female SCID mice. The mice (one mouse par PDX model) were enrolled individually for efficacy studies when the volume of the tumor reached a specific size range (150-250 mm³).

For the evaluation of anti-tumor activity of conjugates, the animals were weighed and the tumors were measured 2 times weekly by caliper. A dosage producing a 20% weight loss at nadir (mean of group) or 10% or more drug deaths, was considered an excessively toxic dosage. Animal body weights included the tumor weights. Tumor volume was calculated using the formula mass (mm³)=[length (mm)×width (mm)2]/2.

The criteria of evaluation of the response of the solid tumors to treatments were based on the criteria RECIST (Response Evaluation Criteria In Solid Tumors) used in clinic and was specifically defined for the SMT project. The tumor response to the various treatments was determined by the difference of tumor volume between a time t (Vt) and in the daytime of starting up of the study t0 (V0) according to the RTV formula=Vt−V0)/V0×100. The best response was determined as the minimal value of the RTV determined at least 5 days after the treatment and noted: best response.

According to the criteria RECIST, the best answer can be qualified as Complete Response (CR): Disappearance of tumor; Partial Response (PR): At least a 30% decrease in the tumor volume compared to baseline; Progressive Disease (PD): More than 20% increase in the tumor volume compared to baseline; Stable Disease (SD): Neither sufficient shrinkage to qualify for PR nor sufficient increase to qualify for PD:

TABLE 17-1

| Best response | RECIST criteria |
|---|---|
| RTV > 20% | Progressive disease (PD) |
| −30% < RTV ≤ 20% | Stable Disease (SD) |
| RTV ≤ −30% | Partial Response (PR) |
| RTV = −100% | Complete Response (CR) |

Using a single administration schedule at 10 mg/kg, the ADC1 DAR 8 did not induce toxicity.

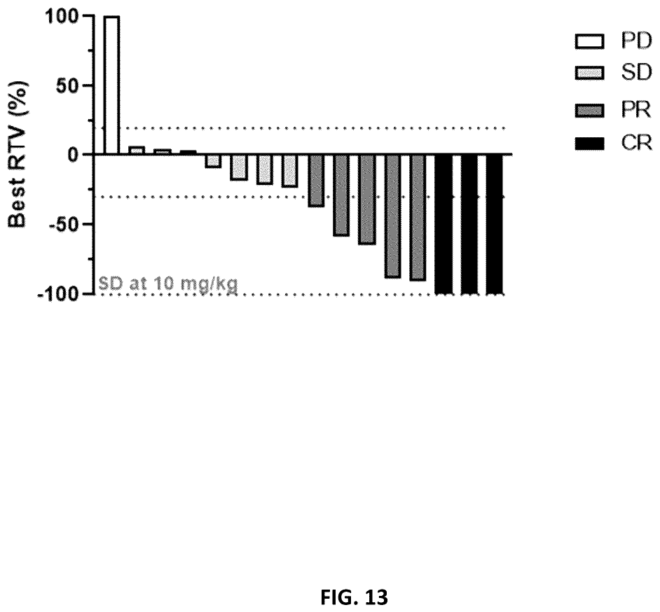

As shown in FIG. 13, from 16 PDX models assessed (11 primary colorectal tumors, 4 metastatic tumors and 1 carcinosis tumor), we observed 3 complete response (18.8%) and 5 partial response (31.2%) i.e. 8 objective responses (50%), 7 stable disease (43.8%), and 1 progressive disease (6.2%). In FIG. 13, the response was determined by comparing tumor volume change at time t to its baseline with ΔRTV=(Vt−V0)/V0×100; Criteria for response were adapted from RECIST clinical criteria; Complete Response (CR): Disappearance of tumor; Partial Response (PR): At least a 30% decrease in the tumor volume compared to baseline; Progressive Disease (PD): At least a 20% increase in the tumor volume compared to baseline; Stable Disease (SD): Neither sufficient shrinkage to qualify for PR nor sufficient increase to qualify for PD. PDX models are sorted by increasing sensitivity to ADC1 DAR 8.

Therefore, ADC1 with DAR 8 was effective for treating gastric tumors.

### Biological Example 18: Rat Toxicity Study of ADC 1 with a DAR of 8 and ADC1 with a DAR of 4

To characterize the toxicity of an antibody that binds to CEACAM5 and is conjugated to a camptothecin linker as both 8-load ADC (e.g., ADC1 with a DAR of 8) and 4-load ADC (e.g., ADC1 with a DAR of 4), and to evaluate exposure of the ADCs, repeat-dose intravenous toxicity study of ADC1 with a DAR of 8 and ADC1 with a DAR of 4 in female rats was conducted, and toxicokinetics and calculated DAR were evaluated.

Weekly intravenous administration of ADC1 with a DAR of 8 at 30 or 50 mg/kg or ADC1 with a DAR of 4 at 50 and 75 mg/kg for 4 weeks to female Sprague Dawley rats was tolerated. Clinical pathology changes were generally minimal, inconsistently related to dose and time point, and included increased neutrophils, monocytes, and platelets. Anatomic pathology test article-related findings on day 29 were observed in lymphoid organs (mandibular lymph nodes, spleen, and/or thymus; minimal to severe lymphoid depletion; all dose groups) and kidney (minimal tubule degeneration; only happened in the group dosed with ADC1 with a DAR of 4 at 75 mg/kg). By day 50 the lymphoid and renal findings fully or partially reversed, while in the lungs minimal to mild alveolar macrophage aggregation was observed in the group dosed with ADC1 with a DAR of 8 at 50 mg/kg, the group dosed with ADC1 with a DAR of 4 at 50 mg/kg, and the group dosed with ADC1 with a DAR of 4 at 75 mg/kg (correlated to increased lung/total-body-weight ratios in the group dosed with ADC1 with a DAR of 4 at 75 mg/kg). Focal skin erosion/ulceration and/or inflammation was observed in two animals in the group dosed with ADC1 with a DAR of 8 at 50 mg/kg and 1 animal in the group dosed with ADC1 with a DAR of 4 at 75 mg/kg, and a single animal in the group dosed with ADC1 with a DAR of 8 at 50 mg/kg had focal mild lung interstitial mononuclear infiltrates; the relationship to test-article is uncertain.

In summary, ADC1 with a DAR of 8 was well tolerated in rats after repeated administration of 30 and 50 mg/kg/day, Q1W×4. The ADC1 with DAR 8 induced a disease control rate of 95% and an overall response rate of 50% following a single dose of 10 mg/kg.

### Biological Example 19: The Bystander Effect of ADC1 with a DAR of 8 for a Decreasing Proportion of CEACAM5+ Cells

NLR-MKN45-KO^{CAM5} generated as described in Biological Example 5 vitro were mixed to parental MKN45 cell line in a range of proportions starting from 5, 000 cells of parental MKN45 cell line plus 5, 000 cells of NLR-MKN45-KO^{CAM5} (50%/50%) down to 100 cells of parental MKN45 cell line plus 9, 900 cells of NLR-MKN45-KO^{CAM5} (1%/99%). Cells were plated in culture medium in a 96-well plate. 5 nM of ADC1 were added to 3 wells and number of NLR-MKN45-KO^{CAM5} was counted every 6 hours for 72 hours using an Incucyte® S3 IC50534 (Essen Biosciences/Sartorius).

The results are summarized in Table 19-1. Through the bystander effect, ADC1 induced a growth inhibition of NLR-MKN45-KO^{CAM5} which was proportional to the amount of active metabolite released by co-cultured parental MKN45 cells. NLR-MKN45-KO^{CAM5} growth inhibition decreased from 49.8% when both cell types are co-cultured at equal proportion (50%/50%) down to 10.7% when 1% of parental MKN45 cells are mixed to 99% of NLR-MKN45-KO^{CAM5}.

TABLE 19-1

| % of MKN45/% of NLR-MKN45-KO^{CAM5} | 50/50 | 30/70 | 20/80 | 10/90 | 5/95 | 1/99 |
|---|---|---|---|---|---|---|
| AUC of NLR-MKN45-KO^{CAM5} | 13363 | 15040 | 16189 | 17686 | 20412 | 23891 |
| | 13241 | 14853 | 16582 | 18648 | 21108 | 22997 |
| | 13298 | 15182 | 15519 | 19182 | 19740 | 24119 |
| Mean | 13301 | 15025 | 16097 | 18505 | 20420 | 23669 |
| % of MKN45-KO^{CAM5} growth inhibition | 49.8% | 43.3% | 39.2% | 30.1% | 22.9% | 10.7% |

Mean AUC of mix of cells not treated by ADC1: 26491

### Biological Example 20: Rate of Internalization in MKN45 Tumor Cells of ADC1 with a DAR of 8

Internalization capability of the ADC1 was investigated using the Incucyte® live-cell technology (Essen Biosciences/Sartorius) and the pH-sensitive FabFluor-pH Red. In culture medium (pH close to 7), the pH-Red dye has little fluorescence. When the complex of ADC1 and FabFluor-pH Red is internalized by the cells in culture, the pH-Red molecules emit strong fluorescence as it reaches acidic compartments like endosomes and lysosomes. The observed fluorogenic signals are quantified.

A rapid time-dependent increase of internalization was observed with ADC1 but not with Medium (cells only) control, with a t₁/2 [95% CI] of 4.49 h [4.219; 4.776].

TABLE 20-1
in vitro Evaluation of ADC1 t1/2 of internalization

| | t_{1/2} (hours) | | |
|---|---|---|---|
| Experiment | By replicate | Mean | 95% confidence interval |
| Experiment 1 | 4.54 | 4.59 | [4.106; 5.071] |
| | 4.80 | | |
| | 4.42 | | |
| Experiment 2 | 4.64 | 4.52 | [4.222; 4.813] |
| | 4.40 | | |
| | 4.51 | | |
| Experiment 3 | 4.35 | 4.37 | [4.241; 4.504] |
| | 4.33 | | |
| | 4.43 | | |

### Biological Example 21: In Vivo Efficacy Evaluation of ADC1 with a DAR of 8 in a Panel of 19 Gastric Patient-Derived Xenograft Models (19 Adenocarcinoma Including 2 Signet-Ring Cell, 2 Metastatic Tumors and 1 Gastric-Esophageal Junction Tumor) Under Single Mouse Trial Format

The Single Mouse Trial (SMT) consists in use of one animal per patient-derived xenograft (PDX) model per treatment arm to evaluate the efficacy of antitumor agents on a larger screening scope, more representative of heterogeneous patient response.

The ADC1 DAR of 8 was evaluated at 10 mg/kg in a panel of 19 gastric PDX models for gastric cancer. The dose was given in mg/kg. It was administered by a single intravenous (IV) bolus injection, on measurable PDX tumors implanted s.c in female immunodeficient mice. The mice (one mouse per PDX model) were enrolled individually for efficacy studies when the volume of the tumor reached a specific size range (150-250 mm³). For each PDX model, one mouse was left untreated, as a control, to monitor tumor growth but was not used for the activity analysis.

For the evaluation of anti-tumor activity of conjugates, the animals were weighed, and the tumors were measured 2 times weekly by caliper. A dosage producing a 20% weight loss at nadir (mean of group) or 10% or more drug deaths, was considered an excessively toxic dosage. Animal body weights included the tumor weights. Tumor volume was calculated using the formula mass (mm³)=[length (mm)×width (mm)2]/2.

The criteria of evaluation of the response of the solid tumors to treatments were based on the criteria RECIST (Response Evaluation Criteria In Solid Tumors) used in clinical trials and was specifically defined for the SMT project. The tumor response to the various treatments was determined by the difference of tumor volume between a time t (Vt) and at the start of the study t0 (V0) according to the RTV formula=(Vt−V0)/V0×100. The best response was determined as the minimal value of the RTV determined at least 5 days after the treatment and noted: best response.

According to the RECIST criteria, the best response is Complete Response (CR): Disappearance of tumor; Partial Response (PR): At least a 30% decrease in the tumor volume compared to baseline; Progressive Disease (PD): More than 20% increase in the tumor volume compared to baseline; Stable Disease (SD): Neither sufficient shrinkage to qualify for PR nor sufficient increase to qualify for PD:

TABLE 21-1

| Best response | RECIST criteria |
|---|---|
| RTV > 20% | Progressive disease (PD) |
| −30% < RTV ≤ 20% | Stable Disease (SD) |
| RTV ≤ −30% | Partial Response (PR) |
| RTV = −100% | Complete Response (CR) |

Using a single administration schedule at 10 mg/kg, the ADC1 did not induce toxicity.

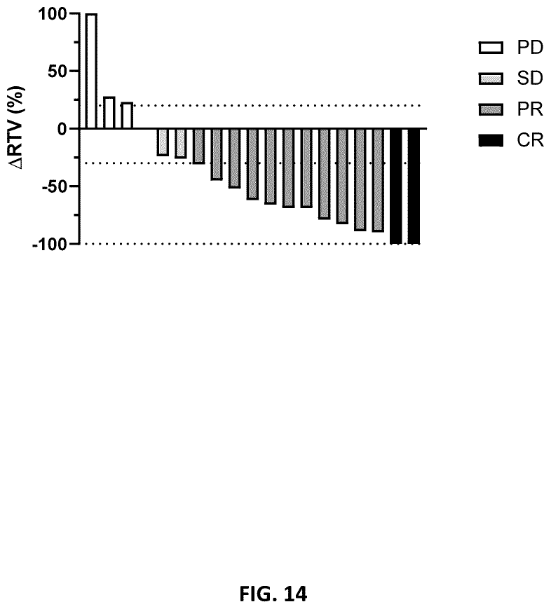

FIG. 14 shows the evaluation of the anti-tumor activity of ADC1 in a panel of 19 gastric patient-derived xenograft models under Single Mouse Trial—Best relative tumor shrinkage. As shown in FIG. 14, from 19 gastric PDX models assessed, we observed 2 complete response (10.5%) and 11 partial response (58%) i.e., 13 objective responses (68%), 3 stable disease (16%), and 3 progressive disease (16%).

In FIG. 14, The response was determined by comparing tumor volume change at time t to its baseline with ΔRTV=(Vt−V0)/V0×100; Criteria for response were adapted from RECIST clinical criteria; Complete Response (CR): Disappearance of tumor; Partial Response (PR): At least a 30% decrease in the tumor volume compared to baseline; Progressive Disease (PD): At least a 20% increase in the tumor volume compared to baseline; Stable Disease (SD): Neither sufficient shrinkage to qualify for PR nor sufficient increase to qualify for PD. PDX models are sorted by increasing sensitivity to ADC1.

Therefore, ADC1 is effective for reducing tumor volumes of gastric cancer.

### Biological Example 22: In Vivo Efficacy Evaluation of ADC1 with a DAR of 8 in a Panel of 31 Lung Patient-Derived Xenograft Models (NSCLC, Nsq-NSCLC and SCLC) Under Single Mouse Trial Format

Efficacy evaluation was performed as reported in Biological Example 21. The ADC1 DAR of 8 was evaluated at 10 mg/kg in a panel of 19 gastric PDX models for lung cancer.

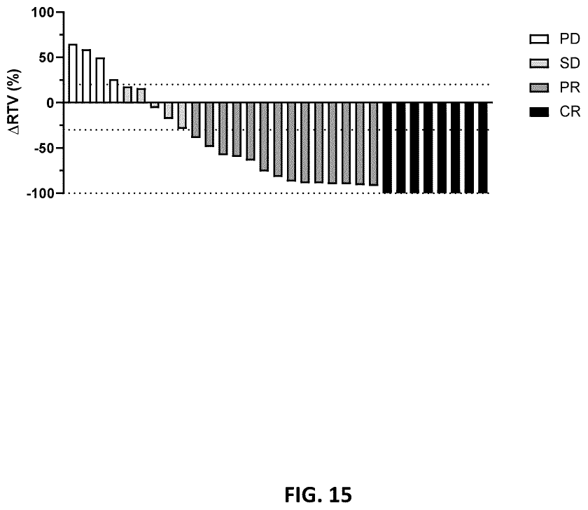

FIG. 15 shows the evaluation of the anti-tumor activity of ADC1 in a panel of 19 gastric patient-derived xenograft models under Single Mouse Trial—Best relative tumor shrinkage. As shown in FIG. 15, using a single administration schedule at 10 mg/kg, the ADC1 did not induce toxicity. From 31 lung PDX models assessed (16 Sq-NSCLC, 13 nsq-NSCLC, 2 SCLC), we observed 8 complete response (26%) (4 Sq-NSCLC, 3 nsq-NSCLC, 1 SCLC) and 14 partial response (45%) (1 SCLC, 6 nsq-NSCLC, 7 sq-NSCLC) i.e., 22 objective responses (71%), 5 stable disease (16%) (2 nsq-NSCL, 3 sq-NSCL), and 4 progressive disease (13%) (2 nsq-NSCLC+2 sq-NSCLC).

In FIG. 15, the response was determined by comparing tumor volume change at time t to its baseline with ΔRTV=(Vt−V0)/V0×100; Criteria for response were adapted from RECIST clinical criteria; Complete Response (CR): Disappearance of tumor; Partial Response (PR): At least a 30% decrease in the tumor volume compared to baseline; Progressive Disease (PD): At least a 20% increase in the tumor volume compared to baseline; Stable Disease (SD): Neither sufficient shrinkage to qualify for PR nor sufficient increase to qualify for PD. PDX models are sorted by increasing sensitivity to ADC1.

This demonstrates that ADC1 is effective for reducing tumor volumes in lung cancer.

### Biological Example 23: In Vivo Efficacy Evaluation of ADC1 with a DAR of 8 Against Pancreas Patient-Derived Xenograft Tumor IM-PAN-011 (mPDAC) Implanted Subcutaneously in Female SCID Mice

The ADC1 with a DAR of 8 was evaluated at 3 doses against measurable pancreas patient-derived xenograft (PDX) tumor IM-PAN-011 tumors implanted subcutaneously (s.c) in female SCID mice. IM-PAN-011 is a metastatic Pancreatic ductal adenocarcinoma (PDAC). Control groups were left untreated. The doses conjugates were given in mg/kg. They were administered at 10, 3 and 1 mg/kg by a single intravenous (IV) bolus injection, on day 21 after tumor implantation.

For the evaluation of anti-tumor activity of conjugates, the animals were weighed, and the tumors were measured 2 times weekly by caliper. A dosage producing a 20% weight loss at nadir (mean of group) or 10% or more drug deaths, was considered an excessively toxic dosage. Animal body weights included the tumor weights. Tumor volume was calculated using the formula mass (mm³)=[length (mm)×width (mm)2]/2. The primary efficacy end points are ΔT/ΔC, percent median regression, partial and complete regressions (PR and CR).

Changes in tumor volume for each treated (T) and control (C) were calculated for each tumor by subtracting the tumor volume on the day of first treatment (staging day) from the tumor volume on the specified observation day. The median ΔT was calculated for the treated group and the median ΔC is calculated for the control group. Then the ratio ΔT/ΔC was calculated and expressed as a percentage:

ΔT/ΔC=(delta T/delta C)×100

The dose was considered as therapeutically active when ΔT/ΔC is lower than or equal to 40% and very active when ΔT/ΔC is lower than or equal to 10%. If ΔT/ΔC is lower than 0, the dose was considered as highly active, and the percentage of regression was dated (Plowman J, Dykes D J, Hollingshead M, Simpson-Herren L and Alley M C. Human tumor xenograft models in NCI drug development. In: Feibig H H B A, editor. Basel: Karger.; 1999p 101-125):

% tumor regression was defined as the % of tumor volume decrease in the treated group at a specified observation day compared to its volume on the first day of first treatment.

At a specific time point and for each animal, % regression was calculated. The median % regression was then calculated for the group.

$$\% regression ( at t ) = \frac{{volume}_{t 0} - {volume}_{t}}{{volume}_{t 0}} \times 100$$

Partial regression (PR): Regressions were defined as partial if the tumor volume decreases to 50% of the tumor volume at the start of treatment.

Complete regression (CR): Complete regression was achieved when tumor volume=0 mm³ (CR is considered when tumor volume cannot be recorded).

Using a single administration schedule at 1, 3 and 10 mg/kg, the conjugate tested in this study did not induce toxicity.

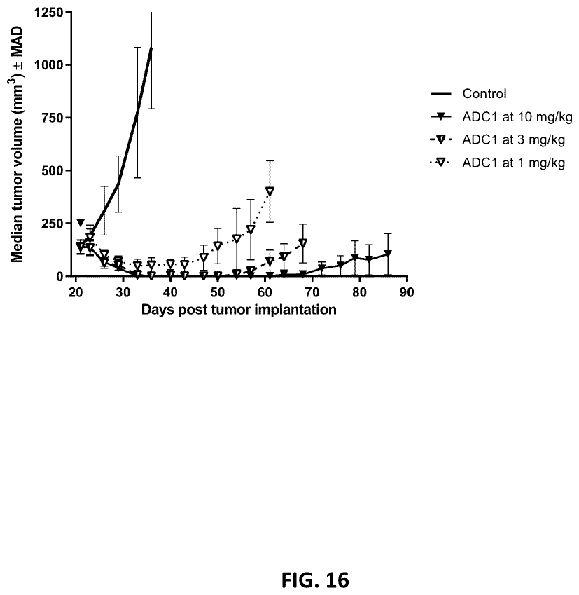

The ADC1 with a DAR of 8 was highly active at the 3 tested doses mg/kg with a ΔT/ΔC less than 0%, a tumor regression of 100%, 6 PR/6 and 6 CR/6 for the dose of 10 mg/kg, with a ΔT/ΔC less than 0%, a tumor regression of 100%, 6 PR/6 and 5 CR/6 for the dose of 3 mg/kg and with a ΔT/ΔC less than 0%, a tumor regression of 73%, 5 PR/6 and 2 CR/6 for the dose of 1 mg/kg. The results are further summarized in FIG. 16 and Table 22-1.

TABLE 22-1
Evaluation of the anti-tumor activity of ADC1 against pancreas
patient-derived xenograft tumor IM-PAN-011 in SCID female mice.

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 36) | (D 36) | PR | CR | Comments |
| ADC1 | IV (10 | 10 | 21 | 0/6 | −3.07 (D 43) | <0 | 100 | 6/6 | 6/6 | Highly active |
| | mL/Kg) | 3 | 21 | 0/6 | −2.93 (D 50) | <0 | 100 | 6/6 | 5/6 | Highly active |
| | | 1 | 21 | 0/6 | −2.45 (D 54) | <0 | 73 | 5/6 | 2/6 | Highly active |
| Control | — | — | — | — | −2.17 (D 36) | — | — | — | — | — |

ΔT/ΔC = ratio of medians of tumor volume changes from baseline between treated and control groups;
PR = Partial regression;
CR = Complete regression

### Biological Example 24: In Vivo Efficacy Evaluation of ADC1 with a DAR of 8 Against Pancreas Patient-Derived Xenograft Tumor SA-PAN-0077 (PDAC) Implanted Subcutaneously in Female SCID Mice

The ADC1 with a DAR of 8 was evaluated at 3 doses against measurable pancreas PDX SA-PAN-0077 tumors implanted s.c in female SCID mice. Control groups were left untreated. The doses conjugates were given in mg/kg. They were administered at 10, 3 and 1 mg/kg by a single intravenous (IV) bolus injection, on day 29 after tumor implantation.

Toxicity and efficacy evaluation were performed as reported in Biological Example 23.

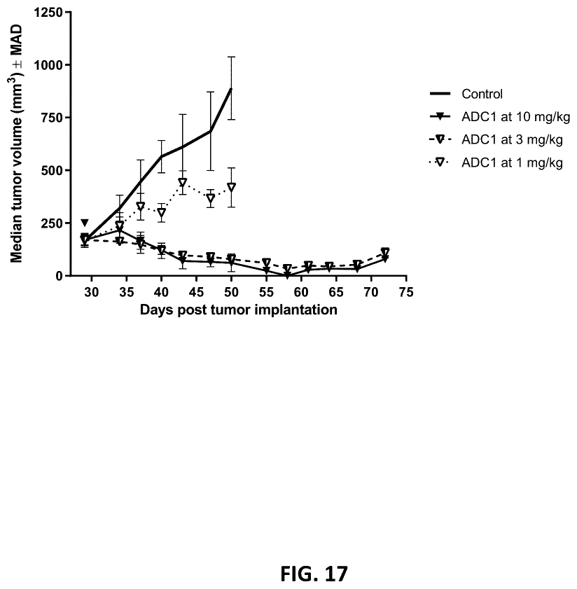

Using a single administration schedule at 1, 3 and 10 mg/kg, the conjugate tested in this study did not induce toxicity. The ADC1 was highly active at 10 and 3 mg/kg with a ΔT/ΔC less than 0%, and with a tumor regression of 100%, 6 PR/6 and 5 CR/6 and with a tumor regression of 80%, 6 PR/6 and 1 CR/6, respectively. It was active at 1 mg/kg with a ΔT/ΔC equal to 36%. From these results, ADC1 was usable to develop a therapeutic ADC in CRC indication. The results are further summarized in FIG. 17 and Table 23-1.

TABLE 23-1
Evaluation of the anti-tumor activity of ADC1 against pancreas patient-
derived xenograft tumor, SA-PAN-0077 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 50) | (D 58) | PR | CR | Comments |
| ADC1 | IV (10 | 10 | 29 | 0/6 | −5.18 (D 37) | <0 | 100 | 6/6 | 5/6 | Highly active |
| | mL/Kg) | 3 | 29 | 0/6 | −4.04 (D 40) | <0 | 80 | 6/6 | 1/6 | Highly active |
| | | 1 | 29 | 0/6 | −0.18 (D 37) | 36 | — | 0/6 | 0/6 | Active |
| Control | — | — | — | — | −0.54 (D 35) | — | — | — | — | — |

ΔT/ΔC = ratio of medians of tumor volume changes from baseline between treated and control groups;
PR = Partial regression;
CR = Complete regression

### Biological Example 25: In Vivo Efficacy Evaluation of ADC1 with a DAR of 8 Against Pancreas Patient-Derived Xenograft Tumor IM-PAN-0006 (PDAC) Implanted Subcutaneously in Female SCID Mice

The ADC1 with a DAR of 8 was evaluated at 3 doses against measurable pancreas PDX IM-PAN-006 tumors implanted s.c in female SCID mice. Control groups were left untreated. The doses conjugates were given in mg/kg. They were administered at 10, 3 and 1 mg/kg by a single intravenous (IV) bolus injection, on day 29 after tumor implantation.

Toxicity and efficacy evaluation were performed as reported in Biological Example 23.

Using a single administration schedule at 10, 3 and 1 mg/kg, the conjugate tested in this study did not induce toxicity.

The ADC1 was highly active at 10 and 3 mg/kg with a ΔT/ΔC less than 0%, and with a tumor regression of 100%, 6 PR/6 and 5 CR/6 and with a tumor regression of 75%, 6 PR/6 and 1 CR/6, respectively. It was inactive at 1 mg/kg with a ΔT/ΔC equal to 44%.

From these results, ADC1 was usable to develop a therapeutic ADC in pancreas indication.

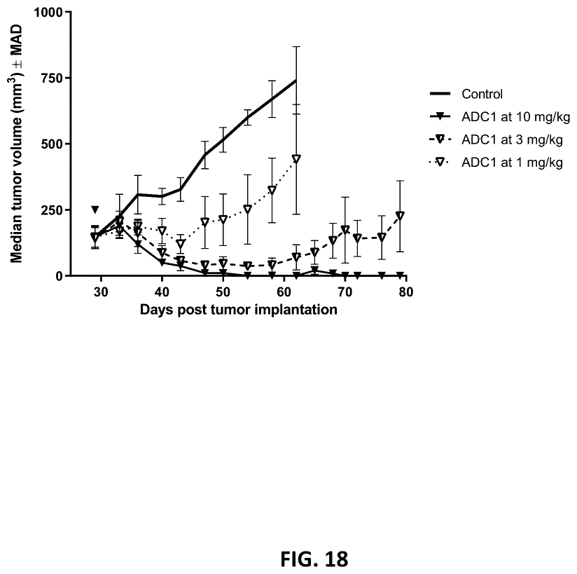

The results are further summarized in Table 25-1 and FIG. 18.

TABLE 25-1
Evaluation of the anti-tumor activity of ADC1 against pancreas
patient-derived xenograft tumor, IM-PAN-006 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 62) | (day) | PR | CR | Comments |
| ADC1 | IV (10 | 10 | 29 | 0/6 | −2.15 (D 40) | <0 | 100 (D 62) | 6/6 | 5/6 | Highly active |
| | mL/Kg) | 3 | 29 | 0/6 | +1.57 (D 40) | <0 | 75 (D 58) | 5/6 | 1/6 | Highly active |
| | | 1 | 29 | 0/6 | −0.72 (D 43) | 40 | — | 0/6 | 0/6 | Inactive |
| Control | — | — | — | — | −2.20 (D 43) | — | — | — | — | — |

ΔT/ΔC = ratio of medians of tumor volume changes from baseline between treated and control groups;
PR = Partial regression;
CR = Complete regression

### Biological Example 26: In Vivo Efficacy Evaluation of ADC1 with a DAR of 8 Against Pancreas Patient-Derived Xenograft Tumor IM-PAN-003 (PDAC) Implanted Subcutaneously in Female SCID Mice

The ADC1 with a DAR of 8 was evaluated at 3 doses against measurable pancreas PDX IP-PAN-003 tumors implanted s.c in female SCID mice. Control groups were left untreated. The doses conjugates were given in mg/kg. They were administered at 10, 3 and 1 mg/kg by a single intravenous (IV) bolus injection, on day 21 after tumor implantation.

Toxicity and efficacy evaluation were performed as reported in Biological Example 23.

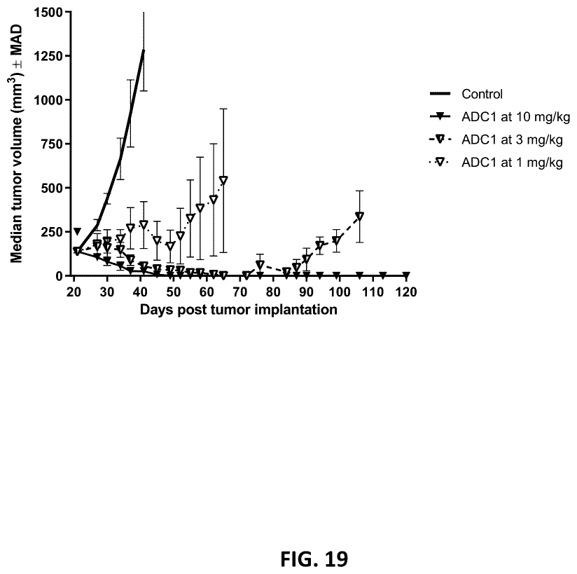

Using a single administration schedule at 1, 3 and 10 mg/kg, the conjugate tested in this study did not induce toxicity. The ADC1 was highly active at 10 and 3 mg/kg with a ΔT/ΔC less than 0%, and with a tumor regression of 100%, 6 PR/6 and 6 CR/6 for both doses. It was active at 1 mg/kg with a ΔT/ΔC equal to 13% and with 1 PR/6. From these results, ADC1 was usable to develop a therapeutic ADC in CRC indication. The results are further summarized in Table 26-1 and FIG. 19.

TABLE 26-1
Evaluation of the anti-tumor activity of ADC1 against pancreas
patient-derived xenograft tumor, IM-PAN-003 in SCID female mice

| | | | | | Mean body weight | | | | | |
|---|---|---|---|---|---|---|---|---|---|---|
| | Route/ | | | Drug | change in % | Median | Median % of | | | |
| | Dosage | Dosage | Schedule | death | per mouse at | ΔT/ΔC | regression | Regressions | | |
| Agent | in mL/kg | in mg/kg | in days | (Day) | nadir (Day) | in % (D 41) | (Day) | PR | CR | Comments |
| ADC1 | IV (10 | 10 | 21 | 0/8 | −3.90 (D 27) | <0 | 100 (D 49) | 6/6 | 6/6 | Highly active |
| | mL/Kg) | 3 | 21 | 0/8 | −2.73 (D 27) | <0 | 100 (D 65) | 6/6 | 6/6 | Highly active |
| | | 1 | 21 | 0/8 | −1.48 (D 24) | 13 | — | 1/6 | 0/6 | Active |
| Control | — | — | — | — | −2.98 (D 24) | — | — | — | — | — |

ΔT/ΔC = ratio of medians of tumor volume changes from baseline between treated and control groups;
PR = Partial regression;
CR = Complete regression

### Biological Example 27: An Open-Label Phase 1 Study to Investigate ADC1 with a DAR of 8 in Adults with Advanced Solid Tumors

An open-label phase 1 clinical trial studying advanced solid tumors is carried out as described herein using ADC1 with a DAR of 8. This study has 3 parts. Part A and optional Part B of the study identifies a suitable dose of ADC1 with a DAR of 8. Part C uses the information from Parts A and B to determine if ADC1 with a DAR of 8 is safe and if it is effective to treat solid tumor cancers.

The conditions of focus include Colorectal Neoplasms, Carcinoma, Non-Small-Cell Lung, Stomach Neoplasms, Pancreatic Ductal Adenocarcinoma, Gastroesophageal Junction Adenocarcinoma, and Small Cell Lung Carcinoma.

ADC1 with a DAR of 8 is administered as a monotherapy intravenously.

The primary outcome measures used for this study include:
Number of participants with adverse events (AEs). An AE is any untoward medical occurrence in a clinical study participant, temporally associated with the use of study intervention, whether or not considered related to the study intervention. The time frame of evaluation is through 30-37 days after the last study treatment, up to approximately 2 years;
Number of participants with laboratory abnormalities. The time frame of evaluation is through 30-37 days after the last study treatment, up to approximately 2 years;
Number of dose modifications due to AEs. The time frame of evaluation is the end of treatment up to approximately 2 years;
Number of participants with dose-limiting toxicities (DLTs). The time frame of evaluation is up to 28 days; and
Number of participants with DLTs by dose level. The time frame of evaluation is up to 28 days.

The secondary outcome measures used for this study include:
Pharmacokinetic (PK) parameter—Area under the concentration-time curve (AUC). The AUC is to be summarized using descriptive statistics. The time frame of evaluation is through 30-37 days after the last study treatment, up to approximately 2 years;
PK parameter—Maximum concentration (Cₘₐₓ). The time frame of evaluation is through 30-37 days after the last study treatment, up to approximately 2 years;
PK parameter—Time to maximum concentration (Tₘₐₓ). The time frame of evaluation is through 30-37 days after the last study treatment, up to approximately 2 years;
PK parameter—Trough concentration (C_{trough}). The time frame of evaluation is through 30-37 days after the last study treatment, up to approximately 2 years;
Number of participants with antidrug antibodies (ADAs). The time frame of evaluation is through 30-37 days after the last study treatment, up to approximately 2 years;
Objective response rate (ORR): The objective response rate (ORR) is defined as the proportion of participants with complete response (CR) or partial response (PR) which is subsequently confirmed as assessed according to Response Evaluation in Solid Tumors (RECIST) v1.1. The time frame of evaluation is through end of study and up to approximately 2 years;
Best response: The best response for a participant is determined by the order of confirmed CR, confirmed PR, stable disease (SD), progressive disease (PD), not evaluable (NE) or not applicable (NA) per RECIST v1.1. The time frame of evaluation is through end of study and up to approximately 2 years;
Duration of response (DOR). DOR is defined as the time from start of the first documentation of objective tumor response (CR or PR) to the first documentation of tumor progression per RECIST v1.1 or to death due to any cause. The time frame of evaluation is through end of study and up to approximately 2 years;
Progression-free survival (PFS). PFS is defined as the time from start of ADC1 with a DAR of 8 to first documentation of disease progression (based on radiographic assessments per RECIST v1.1) or death due to any cause, whichever comes first. The time frame of evaluation is through end of study and up to approximately 2 years; and
Overall survival (OS). OS is defined as the time from start of ADC1 with a DAR of 8 to date of death due to any cause. The time frame of evaluation is through end of study and up to approximately 2 years.

The inclusion criteria include:
Tumor type:
a. Participants in Part A (dose escalation) must have a histologically- or cytologically-confirmed metastatic or unresectable solid tumor malignancy. Participants must have relapsed, refractory, or progressive disease, and should have no appropriate standard therapy available at the time of enrollment in the judgement of the investigator. Participants must have one of the following tumor types:
   Colorectal cancer (CRC)
   Gastric carcinoma (GC) (including signet-ring cell histology) and gastroesophageal junction adenocarcinoma (GEJ)
   Non-small cell lung cancer (NSCLC), squamous or non-squamous histology
   Pancreatic ductal adenocarcinoma (PDAC)
b. For Part B (dose optimization) and Part C (dose expansion):
   Participants must have a histologically- or cytologically-confirmed metastatic or unresectable solid tumor malignancy.
   The tumor types to be enrolled in dose optimization will be identified from among those specified in dose escalation.
   CRC
      Prior therapy: Participants must have received prior treatment (in 1 or more lines of therapy) containing fluoropyrimidine, oxaliplatin, and irinotecan.
   PDAC
      Prior therapy: Participants must have received 1 prior line of therapy and received no more than 3 prior lines of therapy in the advanced or metastatic setting.
   GC/GEJ
      Prior therapy: Participants must have received prior platinum and fluoropyrimidine-based chemotherapy.
   NSCLC—non-squamous/squamous
      Prior therapy: Participants must have received platinum-based therapy. If eligible and consistent with local standard of care, the participants must have received a PD-1/PD-L1 inhibitor.
   Small cell lung cancer (SCLC)
      Prior therapy: Participants must have received platinum-based therapy for extensive-stage disease and no more than 3 prior lines of therapy.

The inclusion criteria may further include:
Participants enrolled in the following study parts should have a tumor site that is accessible for biopsy(ies) and agree to biopsy(ies) and/or submission of archival tissue;
Participants enrolled should have an Eastern Cooperative Oncology Group (ECOG) Performance Status score of 0 or 1;
Participants enrolled should have a measurable disease per Response Evaluation in Solid Tumors (RECIST) v1.1 at baseline.

The exclusion criteria include:
(i) Previous exposure to CEACAM5-targeted therapy;
(ii) Prior treatment with an antibody-drug conjugate (ADC) with a camptothecin payload;
(iii) History of another malignancy within 3 years before the first dose of study intervention, or any evidence of residual disease from a previously diagnosed malignancy;
(iv) Active cerebral/meningeal disease related to the underlying malignancy. Participants with a history of cerebral/meningeal disease related to the underlying malignancy are allowed if prior central nervous system disease has been treated and the subject is clinically stable (defined as not having received steroid treatment for symptoms related to cerebral/meningeal disease for at least 2 weeks prior to enrollment and with no ongoing related Aes).

Subjects are eligible to be included in the study only if all the following criteria are met:
1. Age 18 years and older at the time of consent.
2. Tumor type:
   a. Subjects in dose escalation experiment must have histologically- or cytologically-confirmed metastatic or unresectable solid tumor malignancy. Subjects must have relapsed, refractory, or progressive disease, and should have no appropriate standard therapy available at the time of enrollment in the judgement of the investigator. Subjects must have one of the following tumor types:
      CRC
      GC (including signet-ring cell histology) and GEJ
      NSCLC, squamous or non-squamous histology
      PDAC
   b. For dose optimization and dose expansion:
      Subjects must have histologically- or cytologically-confirmed metastatic or unresectable solid tumor malignancy.
      The tumor types to be enrolled in dose optimization are identified from among those specified in dose escalation.
      CRC:
         Prior therapy: Subjects must have received prior treatment (in 1 or more lines of therapy) containing fluoropyrimidine, oxaliplatin, and irinotecan. These regimens may have been combined with either an anti-angiogenic mAb (such as bevacizumab) if KRAS mutant or an anti-EGFR mAb (such as cetuximab) if KRAS wild-type if eligible and consistent with local standard of care. If eligible and consistent with local standard of care, subjects with MSI-H tumors must have received a prior PD-1/PD-L1 inhibitor. If eligible and consistent with local standard of care, subjects with BRAFV600E must have received appropriate targeted therapy (e.g., encorafenib-based therapy).
      PDAC:
         Prior therapy: Subjects must have received 1 prior line of therapy and received no more than 3 prior lines of therapy in the advanced or metastatic setting. Therapy received in the neoadjuvant setting will not be counted as a separate line of therapy. If eligible and consistent with local standard of care, subjects with MSI-H tumors must have received a prior PD-1/PD-L1 inhibitor.
      GC/GEJ:
         Prior therapy: Subjects must have received prior platinum and fluoropyrimidine-based chemotherapy. Subjects with known HER2 overexpression must have received prior HER2-targeted therapy. If eligible and consistent with local standard of care must have received a prior PD-1/PD-L1 inhibitor.
      NSCLC—non-squamous:
         Prior therapy: Subjects must have received platinum-based therapy. If eligible and consistent with local standard of care must have received a PD-1/PD-L1 inhibitor. Platinum-based therapy and PD-1/PD-L1 inhibitor may have been given separately or in combination.
         Subjects with tumor genomic mutations/alterations for which approved targeted therapies are available per local standard of care, must have received such therapies.
      NSCLC—squamous:
         Prior therapy: Subjects must have received platinum-based therapy. If eligible and consistent with local standard of care must have received a PD-1/PD-L1 inhibitor. Platinum-based therapy and PD-1/PD-L1 inhibitor may have been given separately or in combination.
      SCLC:
         Prior therapy: Subjects must have received platinum-based therapy for extensive-stage disease and no more than 3 prior lines of therapy.
3. In dose optimization and dose expansion cohorts, subjects with NSCLC nsq histology, NSCLC sq histology, or SCLC must have tumors that express CEACAM5, in ≥1% of tumor cells at an intensity of ≥2+ by central IHC testing.
4. Subjects enrolled in the following study parts should have a tumor site that is accessible for biopsy(ies) and agree to biopsy(ies) and/or submission of archival tissue:
   a. Dose optimization
   b. Disease-specific expansion cohorts
5. An Eastern Cooperative Oncology Group (ECOG) Performance Status score of 0 or 1.
6. Measurable disease per Response Evaluation in Solid Tumors (RECIST) v1.1 at baseline.
7. The following baseline laboratory data:
   Absolute neutrophil count (ANC)≥1500/μL
   Hemoglobin ≥9 g/dL
   Platelet count ≥100,000/μL
   Individual estimated glomerular filtration rate (GFR) ≥60 mL/min/1.73 m² using the Modification of Diet in Renal Disease (MDRD) study equation as applicable
   Serum bilirubin ≤1.5-times upper limit of normal (ULN) or ≤3-times ULN for subjects with Gilbert's disease or documented hepatic tumor involvement
   Alanine aminotransferase (ALT) and aspartate aminotransferase (AST) ≤3-times ULN or ≤5-times ULN for participants with liver metastases
8. Subjects of childbearing potential (as defined in Section 10.4) under the following conditions:
   a. Must have a negative serum pregnancy test (minimum sensitivity 25 mIU/mL or equivalent units of beta human chorionic gonadotropin [β-hCG]) result within 72 hours prior to the first dose of study intervention. Subjects with false positive results and documented verification that the subject is not pregnant are eligible for participation.
   b. Must agree not to try to become pregnant during the study and for at least 7 months after the final dose of ADC1 DAR8.
   c. Must agree not to breastfeed or donate ova, from the time of informed consent and continuing through at least 7 months after the final dose of ADC1 DAR8.
   d. If sexually active in a way that could lead to pregnancy, must consistently use at least 2 acceptable methods of birth control (contraception), at least 1 of which must be highly effective from the time of informed consent and continuing through at least 7 months after the final dose of ADC1 DAR8.
9. Subjects who can get someone pregnant under the following conditions:
   a. Must agree not to donate sperm from the time of informed consent and continuing through at least 4 months after the final dose of ADC1 DAR8.
   b. If sexually active with a person of childbearing potential in a way that could lead to pregnancy, must consistently use at least 2 acceptable methods of birth control (contraception), at least 1 of which must be highly effective from the time of informed consent and continuing through at least 4 months after the final dose of ADC1 DAR8.
   c. If sexually active with a person who is pregnant or breastfeeding, must consistently use a condom from the time of informed consent and continuing through at least 4 months after the final dose of ADC1 DAR8.
10. The subject must provide documented informed consent.

The exclusion criteria include:
1. Known hypersensitivity to any excipient contained in the drug formulation of ADC1 DAR8.
2. Previous exposure to CEACAM5-targeted therapy.
3. Prior treatment with an ADC with a CPT payload, such as Enhertu (trastuzumab deruxtecan) or Trodelvy (sacituzumab govitecan). Prior treatment with irinotecan and other non-ADC topoisomerase inhibitors is allowed.
4. History of another malignancy within 3 years before the first dose of study intervention, or any evidence of residual disease from a previously diagnosed malignancy. Exceptions are malignancies with a negligible risk of metastasis or death (eg, 5-year overall survival (OS) ≥90%), such as adequately treated carcinoma in situ of the cervix, non-melanoma skin carcinoma, localized prostate cancer, ductal carcinoma in situ, or Stage I uterine cancer.
5. Active cerebral/meningeal disease related to the underlying malignancy. Subjects with a history of cerebral/meningeal disease related to the underlying malignancy are allowed if prior central nervous system disease has been treated and the subjects is clinically stable (defined as not having received steroid treatment for symptoms related to cerebral/meningeal disease for at least 2 weeks prior to enrollment and with no ongoing related AEs).
6. Any uncontrolled viral, bacterial, or fungal infection within 2 weeks prior to the first dose of study intervention, unless deemed not clinically significant by the investigator (eg, onychomycosis). Routine antimicrobial prophylaxis is permitted.
7. Positive for hepatitis B by surface antigen expression (HBsAg+) and/or hepatitis B core antibody (HBcAb+); subjects with a negative hepatitis B DNA PCR assay are permitted with appropriate antiviral prophylaxis. Subjects with positive HBsAg+ and/or HBcAb+ but negative hepatitis B DNA should be referred to a hepatologist prior to Cycle 1 Day 1 and should undergo monitoring for hepatitis B reactivation as per the schedule of activities.
8. Active hepatitis C infection (positive by PCR or on antiviral therapy for hepatitis C within the last 6 months). Subjects who have been treated for hepatitis C infection or who have spontaneously recovered are permitted if they have documented sustained virologic response (undetectable hepatitis C virus [HCV] RNA by PCR) of 12 weeks.
9. Known to be positive for human immunodeficiency virus (HIV).
10. Documented history of a cerebral vascular event (stroke or transient ischemic attack), unstable angina, myocardial infarction, congestive heart failure, or cardiac symptoms consistent with New York Heart Association Class III-IV within 6 months prior to the first dose of study intervention.
11. Major surgery within 4 weeks, or minor surgery within 7 days prior to the first dose of study intervention. Participants must have recovered adequately from the toxicity or complications from the surgery prior to starting ADC1 DAR8 treatment. Participants who have planned major surgery during the treatment period must be excluded from the study.
12. Current therapy with other investigational agents or other systemic anti-neoplastic agents.
13. Chemotherapy, radiotherapy, biologics, and/or other antitumor treatment with immunotherapy that is not completed 4 weeks prior to first dose of study intervention, or within 2 weeks prior to first dose of study intervention if the underlying disease has progressed on treatment, after discussion with the medical monitor.
14. Treatment with any prohibited concomitant therapy or procedure.
15. Grade 2 or higher pulmonary disease unrelated to underlying malignancy, or history of Grade 2 or higher drug-induced interstitial lung disease (ILD) or immune checkpoint inhibitor (ICI)-related ILD.
16. Has had clinically significant lung disease requiring systemic corticosteroid treatment within 6 months prior to enrollment (e.g., interstitial pneumonia, pneumonitis, pulmonary fibrosis, and severe radiation pneumonitis) or who are suspected to have such diseases via radiographic imaging and/or functional tests conducted during the screening period.
17. Subjects with active Grade 2 anorexia, nausea or vomiting, and/or signs of intestinal obstruction.
18. History of clinically significant gastrointestinal bleeding, intestinal obstruction, or gastrointestinal perforation within 6 months of initiation of trial treatment.
19. Subjects with a condition requiring systemic treatment with either corticosteroids (>10 mg daily prednisone or equivalent) or other immunosuppressive medications within 2 weeks of first dose of ADC1 DAR8 (inhaled, topical, intraocular, intranasal, and intraarticular steroids are permitted in the absence of active immune disease, and steroid premedication for prevention of hypersensitivity reactions to radiographic contrast is permitted).
20. Estimated life expectancy <12 weeks in the opinion of the investigator.
21. Any clinically significant toxicity with prior therapy that has not returned to baseline or that is NCI CTCAE version 5.0 Grade 2 or higher (with the exception of alopecia) or toxicity deemed clinically significant by the investigator.
22. Live or live-attenuated vaccine(s) (mRNA and replication deficient adenoviral vaccines are not considered attenuated live vaccines) received within 30 days prior to Cycle 1 Day 1.
23. Other serious underlying medical condition that, in the opinion of the investigator, would impair the subject's ability to receive or tolerate the planned treatment and follow-up.

### Biological Example 28: Pharmacokinetic (PK) Profile of ADC1 with a DAR of 8 after a Single Intravenous (3 mg/kg) Administration to Female SCID Mice

### Materials and Methods

### PK Study in Female SCID Mice

The experiment was performed in SCID mice. Three SCID female mice per time point (body weight range of 18-22 g) were used at study start.

An ADC1 with a DAR of 8 stock solution (14.6 mg/mL) was prepared in 20 mM glutamic acid pH4.5 buffer diluted in the same buffer extemporaneously and administered as single intravenous dose of 3 mg/kg into the tail vein with a dose volume of 10 mL/kg. Animals were evaluated utilizing a non-serial sampling approach with sampling at 5 min, 2, 4, 24, 72, 168, 336, 504, 672 hours (0.0035, 0.083, 0.17, 1, 3, 7, 14, 21 and 28 days) across the study duration of 28 days. At each time point, blood was withdrawn from cardiac puncture into K3-EDTA collecting devices. Immediately after collection, blood samples were placed on wet ice and then centrifuged.

The plasma concentrations of Total Antibody-Conjugated-Drug (Total CD) at each time point, were determined by combining Ligand-Binding Assay (LBA) for Total mAb and LC-MS/MS assay for DAR (Drug-Antibody-Ratio) distribution:

The analytical methods were the following:

Ligand Binding Assay (LBA): Capture by anti-CEACAM5-Biotin bound on the streptavidin-beads of Gyrolab xP microstructured discs and detection using Goat Anti-hu-IgGFc-AlexaFluor tracer, before the reading of fluorescence (kexc 633 nm, kemm 650 nm).

LC-MS/MS assay: DAR of ADC1 was determined on pooled mice plasma samples by LC-MS/MS. Anti-CEACAM5 ADC was first purified from plasma using Streptavidin cartridges coated with biotinylated CEACAM5 on the Agilent Assay MAP Bravo. All samples were then reduced and analyzed under denaturing conditions by LC-MS on a mass spectrometer. This analysis led to the individual MS detection of light chains (LC) and heavy chains (HC) (the signal from the (intact) ADC therefore not being measurable). Mass spectrum deconvolution for individual DAR calculation of LC and HC was performed using Waters Biopharmalynx software and DAR averages of the LC and HC with the corresponding percentages were further used for the calculation of the DAR of the ADC, assuming the same probability of all possible combinations of LC/HC conjugated species to occur.

Individual DAR entities concentrations were then calculated for each sampling time point by integrating the total mAb concentration obtained by Ligand Binding Assay (LBA) with the High Resolution Mass Spectrometer (HRMS) normalized ADC DAR response obtained as previously described. Total antibody-conjugated drug concentrations (Total CD) were calculated in molarity considering the mass of the released drug, the number of drugs loaded by DAR entities, and the mass of the DAR entity (e.g., the mass of an ADC with DAR of 4 or DAR of 8).

The Lower Limit of Quantification (LLOQ) value was of 0.2 μg/mL.

### Pharmacokinetics Analysis

Pharmacokinetics parameters were estimated using Phoenix pharmacokinetic software. A non-compartmental approach consistent with the intravenous administration was used for parameter estimation. PK parameters were generated from anti-CEACAM5 Total CD individual concentrations in plasma up to 28 days. Values were expressed with three significant figures. Concentration values reported as Below the Quantification Limit (BLQ) were assigned a value of 0 for descriptive statistics calculation.

### Results

No clinical signs or symptoms were observed during study.

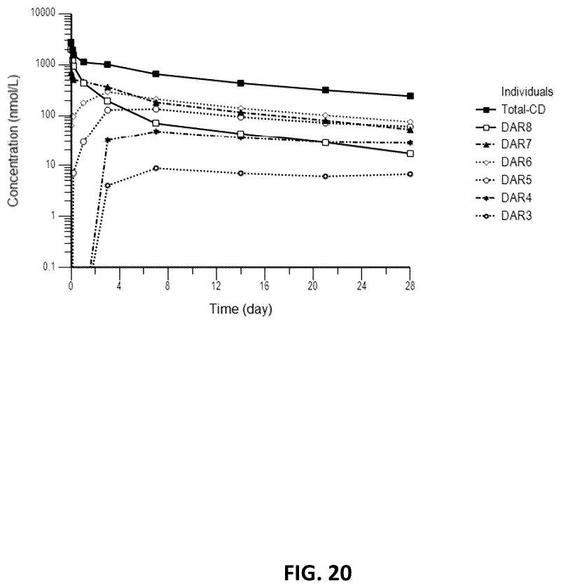

Values of anti-CEACAM5 Total CD plasma concentrations (pg/mL) obtained after a single IV (3 mg/kg) administration of ADC1 with a DAR of 8 to female SCID mice are reported below in Table 28-1 and corresponding plasma concentrations versus time profiles are shown in FIG. 20.

TABLE 28-1
Anti-CEACAM5 Total CD and DAR entities concentrations in plasma following single
intravenous administration of ADC1 with a DAR of 8 to SCID mice at 3 mg/kg

| | | Time (day) | | | | | | | | |
|---|---|---|---|---|---|---|---|---|---|---|
| Measured | Animal | 0.0035 | 0.083 | 0.17 | 1.0 | 3.0 | 7.0 | 14 | 21 | 28 |
| Compound | Id. | Concentrations (μg/mL) | | | | | | | | |
| Total-CD | Pool | 2760 | 1940 | 1560 | 1110 | 1010 | 653 | 434 | 316 | 241 |
| DAR8 | Pool | 1980 | 1240 | 943 | 440 | 193 | 69.6 | 43.5 | 28.6 | 17.2 |
| DAR7 | Pool | 715 | 603 | 510 | 462 | 364 | 181 | 115 | 79.6 | 52.8 |
| DAR6 | Pool | 63.1 | 99.0 | 95.2 | 178 | 293 | 211 | 139 | 101 | 75.5 |
| DAR5 | Pool | BLQ | BLQ | 7.11 | 29.3 | 128 | 134 | 93.5 | 71.5 | 60.6 |
| DAR4 | Pool | BLQ | BLQ | BLQ | BLQ | 31.6 | 48.3 | 35.8 | 29.0 | 28.1 |
| DAR3 | Pool | BLQ | BLQ | BLQ | BLQ | 4.06 | 8.88 | 7.06 | 6.13 | 6.80 |

* Plasma concentrations entities were calculated from total ADC concentrations by LBA;
ND: Not detected;
** BLQ (LOQ evaluated for DAR entities at 0.1 μg/mL)

Pharmacokinetic parameters of anti-CEACAM5 Total CD and DAR entities in plasma after a single intravenous (3 mg/kg) administration of ADC1 with a DAR of 8 are presented below in Table 28-2.

TABLE 28-2
Anti-CEACAM5 Total CD pharmacokinetics parameters in
plasma following single intravenous administration
of ADC1 with a DAR of 8 to SCID mice at 3 mg/kg.

| | | | | | | DAR |
|---|---|---|---|---|---|---|
| | | AUC | CL | | | distri- |
| Measured | C₀/Cₘₐₓ | (day · | (mL/day/ | Vₐₛₛ | t_{1/2z} | bution |
| compound | (nmol/L) | nmol/L) | kg) | (mL/kg) | (day) | % AUC |
| Total-CD | 2800 | 20400 | 8 | 149 | 15 | — |
| DAR8 | 2020 | 3030 | 39 | 341 | 11 | 18 |
| DAR7 | 720 | 5550 | — | — | — | 30 |
| DAR6 | 63.1 | 6020 | — | — | — | 28 |
| DAR5 | NA | 4490 | — | — | — | 17 |
| DAR4 | NA | 2540 | — | — | — | 6 |
| DAR3 | NA | NC | — | — | — | 1 |

NC Not calculated;
NA Not Applicable

After 3 mg/kg intravenous administration, anti-CEACAM5 Total CD concentrations were quantifiable in plasma up to 28 days (last sampling time). Plasma clearance was estimated to 8 mL/day/kg and volume of distribution at steady state was 149 mL/kg leading to a terminal elimination half-life (t₁/2) of around 15 days.

### Biological Example 29: In Vivo Proteolytic Metabolism of ADC1 with a DAR of 8 after a Repeat-Dose Intravenous Administration to Male Non-Human Primates (NHP)

### Material and Methods

### Protelytic Metabolism in Cynomolgus Monkey PK

The NHP experiment was performed in Non-human/Macaca fascicularis (Cynomolgus) monkies. Two animals/sex per time point (body weight range of 2.6-4.3 kg) were used at study start.

An ADC1 with a DAR of 8 stock solution (26.2 mg/mL) was prepared in trehalose/20 mM glutamic acid pH4.5 (7%/93%) buffer and administered as Q2W×3 intravenous dose into the saphenous veins with a dose volume of 2 mL/kg. Animals were evaluated utilizing a serial sampling approach with sampling at 5 min, 2, 4, 6, 24, 48, 72, 168 and 336 hours (0.0035, 0.083, 0.167, 0.25, 1, 2, 3, 7 and 14 days) across the study duration of 29 days. At each time point, blood was withdrawn from the femoral, saphenous and/or cephalic vein into K3-EDTA collecting devices. Immediately after collection, blood samples were placed on wet ice and then centrifuged.

The in vivo proteolytic metabolic profile of ADC1 with a DAR of 8 in plasma samples from NHPs was determined with the following middle-up LC-MS assay:

Middle-up LC-MS assay: Immunocapture on Agilent AssayMAP Bravo Streptavidin cartridges coated with biotinylated CEACAM5 followed by hinge-specific digestion with IdeS enzyme and analysis under denaturing conditions of light chain (Lc) and Fd fragment (Fd) by micro-LC coupled with HRMS on an Orbitrap Exploris 480 mass spectrometer.

Mass spectrum deconvolution for protein assignment was performed using Genedata Expressionist software.

### Results

The main protein subunits detected across all plasma samples were DAR 1 Lc and DAR 3 Fd originating from non-metabolized DAR 8 ADC1 digested with IdeS enzyme. Some lower mass proteolytic metabolites were observed and attributed to DAR 0 Lc as well as DAR 2 and DAR 1 Fd all originating from deconjugation of SGD-7192. Hydrolysis of SGD-7192 conjugated onto Lc and Fd could be precisely assessed and followed for each drug-bearing protein subunits (non-metabolized and metabolites) across the analyzed plasma samples. In addition, higher mass proteolytic metabolites were detected and are currently under identification as hypothetical recombined Lc and Fd chains via interchain disulfide bonds.

### INCORPORATION BY REFERENCE

All references cited herein, including patents, patent applications, scientific papers, textbooks and the like are incorporated by reference in their entirety.

Informal Sequence Listing

| Description | Sequence | SEQ ID NO: |
|---|---|---|
| CDR1-H | GFVFSSYD | 1 |
| CDR-2H | ISSGGGIT | 2 |
| CDR-3H | AAHYFGSSGPFAY | 3 |
| CDR1-L | ENIFSY | 4 |
| CDR2-L | NTR | 5 |
| CDR3-L | QHHYGTPFT | 6 |
| VH | EVQLQESGPGLVKPGGSLSLSCAASGFVFSSYDMSWVRQTPERGL | 7 |
| | EWVAYISSGGGITYAPSTVKGRFTVSRDNAKNTLYLQMNSLTSED | |
| | TAVYYCAAHYFGSSGPFAYWGQGTLVTVSS | |
| VL | DIQMTQSPASLSASVGDRVTITCRASENIFSYLAWYQQKPGKSPK | 8 |
| | LLVYNTRTLAEGVPSRFSGSGSGTDFSLTISSLQPEDFATYYCQH | |
| | HYGTPFTFGSGTKLEIK | |
| Heavy | EVQLQESGPGLVKPGGSLSLSCAASGFVFSSYDMSWVRQTPERGL | 9 |
| chain | EWVAYISSGGGITYAPSTVKGRFTVSRDNAKNTLYLQMNSLTSED | |
| | TAVYYCAAHYFGSSGPFAYWGQGTLVTVSSASTKGPSVFPLAPSS | |
| | KSTSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPELLGGPSVFLFPPKPKDTLMISRTPEVTCVVVDVS | |
| | HEDPEVKFNWYVDGVEVHNAKTKPREEQYNSTYRVVSVLTVLHQD | |
| | WLNGKEYKCKVSNKALPAPIEKTISKAKGQPREPQVYTLPPSRDE | |
| | LTKNQVSLTCLVKGFYPSDIAVEWESNGQPENNYKTTPPVLDSDG | |
| | SFFLYSKLTVDKSRWQQGNVFSCSVMHEALHNHYTQKSLSLSPG | |
| Light chain | DIQMTQSPASLSASVGDRVTITCRASENIFSYLAWYQQKPGKSPK | 10 |
| | LLVYNTRTLAEGVPSRFSGSGSGTDFSLTISSLQPEDFATYYCQH | |
| | HYGTPFTFGSGTKLEIKRTVAAPSVFIFPPSDEQLKSGTASVVCL | |
| | LNNFYPREAKVQWKVDNALQSGNSQESVTEQDSKDSTYSLSSTLT | |
| | LSKADYEKHKVYACEVTHQGLSSPVTKSFNRGEC | |
| Human | MESPSAPPHRWCIPWQRLLLTASLLTFWNPPTTAKLTIESTPFNV | 11 |
| CEACAM | AEGKEVLLLVHNLPQHLFGYSWYKGERVDGNRQIIGYVIGTQQAT | |
| 5 | PGPAYSGREIIYPNASLLIQNIIQNDTGFYTLHVIKSDLVNEEAT | |
| | GQFRVYPELPKPSISSNNSKPVEDKDAVAFTCEPETQDATYLWWV | |
| | NNQSLPVSPRLQLSNGNRTLTLFNVTRNDTASYKCETQNPVSARR | |
| | SDSVILNVLYGPDAPTISPLNTSYRSGENLNLSCHAASNPPAQYS | |
| | WFVNGTFQQSTQELFIPNITVNNSGSYTCQAHNSDTGLNRTTVTT | |
| | ITVYAEPPKPFITSNNSNPVEDEDAVALTCEPEIQNTTYLWWVNN | |
| | QSLPVSPRLQLSNDNRTLTLLSVTRNDVGPYECGIQNELSVDHSD | |
| | PVILNVLYGPDDPTISPSYTYYRPGVNLSLSCHAASNPPAQYSWL | |
| | IDGNIQQHTQELFISNITEKNSGLYTCQANNSASGHSRTTVKTIT | |
| | VSAELPKPSISSNNSKPVEDKDAVAFTCEPEAQNTTYLWWVNGQS | |
| | LPVSPRLQLSNGNRTLTLFNVTRNDARAYVCGIQNSVSANRSDPV | |
| | TLDVLYGPDTPIISPPDSSYLSGANLNLSCHSASNPSPQYSWRIN | |
| | GIPQQHTQVLFIAKITPNNNGTYACFVSNLATGRNNSIVKSITVS | |
| | ASGTSPGLSAGATVGIMIGVLVGVALI | |
| Murine | QLTIESRPFNVAEGKEVLLLAHNVSQNLFGYIWYKGERVDASRRI | 12 |
| CEACAM | GSCVIRTQQITPGPAHSGRETIDFNASLLIQNVTQSDTGSYTIQV | |
| 5 | IKEDLVNEEATGQFRVYPELPKPYITSNNSNPIEDKDAVALTCEP | |
| | ETQDTTYLWWVNNQSLPVSPRLELSSDNRTLTVFNIPRNDTTSYK | |
| | CETQNPVSVRRSDPVTLNVLYGPDAPTISPLNTPYRAGEYLNLTC | |
| | HAASNPTAQYFWFVNGTFQQSTQELFIPNITVNNSGSYMCQAHNS | |
| | ATGLNRTTVTAITVYAELPKPYITSNNSNPIEDKDAVTLTCEPET | |
| | QDTTYLWWVNNQRLSVSSRLELSNDNRTLTVFNIPRNDTTFYECE | |
| | TQNPVSVRRSDPVTLNVLYGPDAPTISPLNTPYRAGENLNLSCHA | |
| | ASNPAAQYFWFVNGTFQQSTQELFIPNITVNNSGSYMCQAHNSAT | |
| | GLNRTTVTAITVYVELPKPYISSNNSNPIEDKDAVTLTCEPVAEN | |
| | TTYLWWVNNQSLSVSPRLQLSNGNRILTLLSVTRNDTGPYECGIQ | |
| | NSESAKRSDPVTLNVTYGPDTPIISPPDLSYRSGANLNLSCHSDS | |
| | NPSPQYSWLINGTLRQHTQVLFISKITSNNNGAYACFVSNLATGR | |
| | NNSIVKNISVSSGDSAPGSSGLSA | |

1. An antibody-drug conjugate that binds to CEACAM5 having the formula of
L-(Q-D)ₚ
or a salt thereof, wherein
L is a Ligand Unit comprising an antibody or antigen binding fragment thereof that binds to CEACAM5;
subscript p is an integer ranging from 1 to 16;
Q is a Linker Unit having a formula selected from the group consisting of:
—Z-A-RL-,—Z-A-RL-Y—,—Z-A-S*-RL-,—Z-A-B(S*)—RL-, —Z-A-S*-RL-Y—, and —Z-A-B(S*)—RL-Y—;
wherein Z is a Stretcher Unit;
A is a bond or a Connector Unit;
B is a Parallel Connector Unit;
S* is a Partitioning Agent;
RL is a glycoside unit;
Y is a Spacer Unit; and
D is a Drug Unit having the formula of: 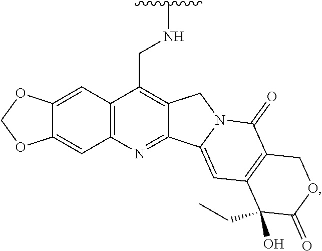 [SMILES: *NCc1c2c(nc3cc4c(cc13)OCO4)-c1cc3c(c(=O)n1C2)COC(=O)[C@]3(O)CC]
wherein the wavy line indicates the site of covalent attachment to Q.

2. An antibody-drug conjugate that binds to CEACAM5 having the formula of
L-(Q-D)ₚ
or a salt thereof, wherein
L is a Ligand Unit comprising an antibody or antigen binding fragment thereof that binds to CEACAM5 comprising
a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8;
subscript p is an integer ranging from 1 to 16;
Q is a Linker Unit;
and D is a Drug Unit, wherein the Drug Unit is a Topoisomerase I inhibitor.

3. The antibody-drug conjugate or salt thereof of claim 1, wherein the antibody or antigen binding fragment thereof comprises
a CDR1-H comprising the amino acid sequence set forth in SEQ ID NO:1;
a CDR2-H comprising the amino acid sequence set forth in SEQ ID NO:2;
a CDR3-H comprising the amino acid sequence set forth in SEQ ID NO:3;
a CDR1-L comprising the amino acid sequence set forth in SEQ ID NO:4;
a CDR2-L comprising the amino acid sequence NTR; and
a CDR3-L comprising the amino acid sequence set forth in SEQ ID NO:63.

4. The antibody-drug conjugate or salt thereof of claim 1, wherein the antibody or antigen binding fragment thereof comprises
a CDR1-H, a CDR2-H, and a CDR3-H of a variable heavy chain domain (VH) comprising the amino acid sequence set forth in SEQ ID NO:7 and
a CDR1-L, a CDR2-L, and a CDR3-L of a variable light chain domain (VL) comprising the amino acid sequence set forth in SEQ ID NO:8.

5. The antibody-drug conjugate or salt thereof of claim 1, wherein the antibody or antigen binding fragment thereof comprises a heavy chain that has at least 80% amino acid sequence identity to the amino acid sequence set forth in SEQ ID NO:9, and a light chain that has at least 80% amino acid sequence identity to the amino acid sequence set forth in SEQ ID NO:10.

6. The antibody-drug conjugate or salt thereof of claim 1, wherein the antibody or antigen binding fragment thereof comprises a heavy chain that has the amino acid sequence set forth in SEQ ID NO:9, and a light chain that has the amino acid sequence set forth in SEQ ID NO:10.

7. The antibody-drug conjugate or salt thereof of claim 2, wherein the antibody or antigen binding fragment thereof comprises a heavy chain that has at least 80% amino acid sequence identity to the amino acid sequence set forth in SEQ ID NO:9, and a light chain that has at least 80% amino acid sequence identity to the amino acid sequence set forth in SEQ ID NO:10.

8. The antibody-drug conjugate or salt thereof of claim 2, wherein the antibody or antigen binding fragment thereof comprises a heavy chain that has the amino acid sequence set forth in SEQ ID NO:9, and a light chain that has the amino acid sequence set forth in SEQ ID NO:10.

9. The antibody-drug conjugate or salt thereof of claim 2, wherein the D is a Drug Unit having the formula of: 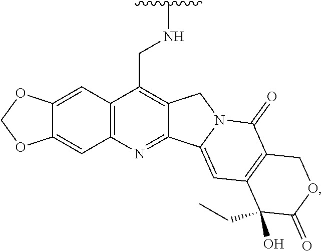 [SMILES: *NCc1c2c(nc3cc4c(cc13)OCO4)-c1cc3c(c(=O)n1C2)COC(=O)[C@]3(O)CC]
wherein the wavy line indicates the site of covalent attachment to Q.

10. The antibody-drug conjugate or salt thereof of claim 1, wherein Q is a Linker Unit having the formula —Z-A-RL-, and wherein RL is a Glucuronide Unit having the formula: 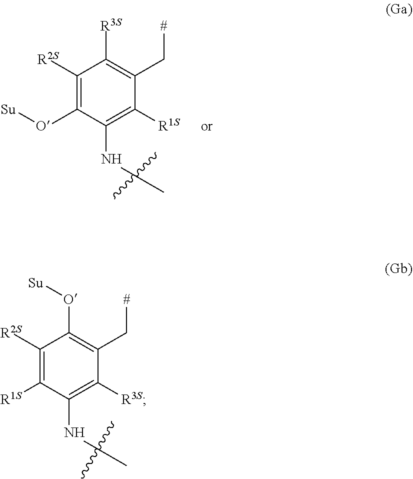
wherein
Su is a sugar moiety;
—O′— is an oxygen glycosidic bond;
R^{1S}, R^{2S} and R^{3S} independently are hydrogen, halogen, —CN, —NO₂, or other electron withdrawing group, or an electron donating group;
the wavy line indicates attachment to Z, either directly or indirectly through A or B or A and B; and
# indicates direct covalent attachment to D or Y.

11. The antibody-drug conjugate or salt thereof of claim 2, wherein Q is a Linker Unit having the formula —Z-A-RL-, and wherein Z is a Stretcher Unit; wherein A is a bond or a Connector Unit; and wherein RL is a Glucuronide Unit having the formula: 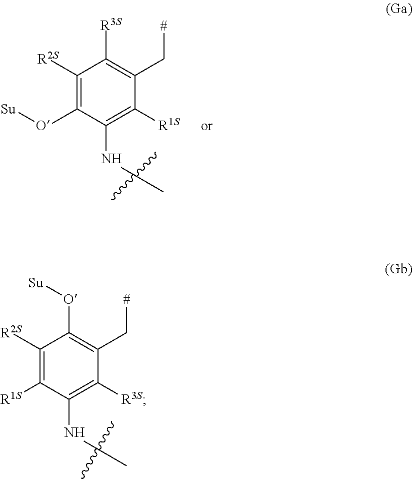
wherein
Su is a sugar moiety;
—O′— is an oxygen glycosidic bond;
R^{1S}, R^{2S} and R^{3S} independently are hydrogen, halogen, —CN, —NO₂, or other electron withdrawing group, or an electron donating group;
the wavy line indicates attachment to Z, either directly or indirectly through A or B or A and B; and
# indicates direct covalent attachment to D or Y.

12. The antibody-drug conjugate or salt thereof of claim 10, wherein the Glucuronide Unit has the formula: 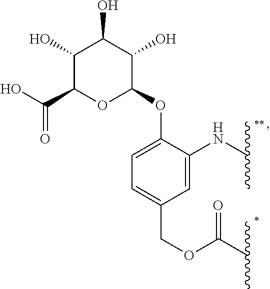 [SMILES: *Nc1cc(COC(*)=O)ccc1O[C@H]1O[C@@H](C(=O)O)[C@H](O)[C@@H](O)[C@@H]1O]
wherein
the wavy line marked with a single asterisk (*) indicates the site of covalent attachment to D; and
the wavy line marked with a double asterisk (**) indicates the site of covalent attachment to the remainder of Q.

13. The antibody-drug conjugate or salt thereof of claim 11, wherein the Glucuronide Unit has the formula: 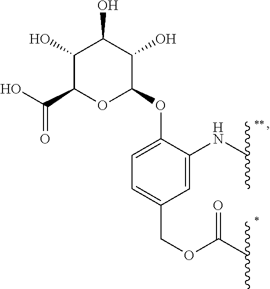 [SMILES: *Nc1cc(COC(*)=O)ccc1O[C@H]1O[C@@H](C(=O)O)[C@H](O)[C@@H](O)[C@@H]1O]
wherein
the wavy line marked with a single asterisk (*) indicates the site of covalent attachment to D; and
the wavy line marked with a double asterisk (**) indicates the site of covalent attachment to the remainder of Q.

14. The antibody-drug conjugate or salt thereof of claim 1, or a salt thereof, wherein Z is 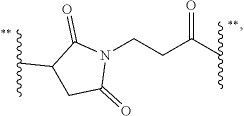 [SMILES: *C(=O)CCN1C(=O)CC(*)C1=O]
optionally having the succinimide ring in hydrolyzed form as a succinic acid amide moiety, wherein the wavy line marked with a double asterisk (**) indicates the site of covalent attachment to the rest of Q; and the wavy line marked with a triple asterisk (***) indicates the point of covalent attachment to a sulfur atom of L.

15. The antibody-drug conjugate or salt thereof of claim 1, wherein A is a Connector Unit and has the formula: 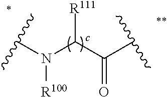
wherein
the wavy line marked with a double asterisk (**) indicates the site of covalent attachment to RL;
the wavy line marked with a single asterisk (*) indicates the point of covalent attachment to Z;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, 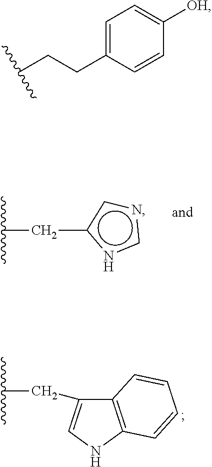
each R¹⁰⁰ is independently selected from the group consisting of hydrogen and —C₁-C₃ alkyl; and
c is an independently selected integer from 1 to 10.

16. The antibody-drug conjugate or salt thereof of claim 15, wherein A has the formula: 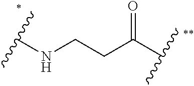 [SMILES: *NCCC(*)=O]
wherein
the wavy line marked with a double asterisk (**) indicates the site of covalent attachment to RL; and
the wavy line marked with a single asterisk (*) indicates the point of covalent attachment to Z.

17. The antibody-drug conjugate or salt thereof claim 1, wherein —Z-A-RL- has the formula: 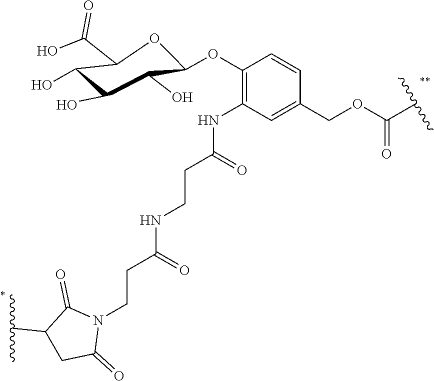
wherein
the wavy line marked with a double asterisk (**) indicates the site of covalent attachment to D; and
the wavy line marked with a single asterisk (*) indicates the point of covalent attachment to L.

18. The antibody-drug conjugate or salt thereof of claim 1, wherein —RL-D- has the formula: 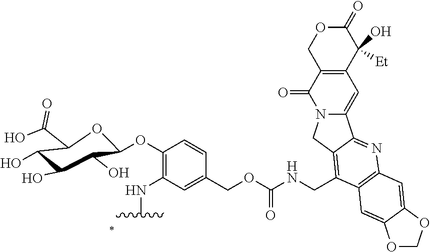
wherein
the wavy line marked with a single asterisk (*) indicates the point of covalent attachment to A.

19. The antibody-drug conjugate or salt thereof of claim 1, wherein -A-RL-D has the formula: 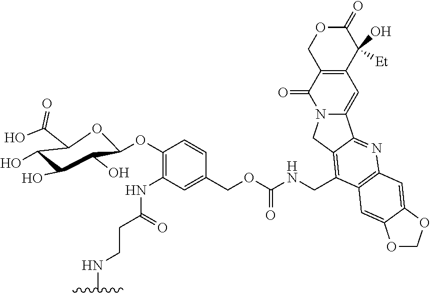
wherein
the wavy line marked with a single asterisk (*) indicates the point of covalent attachment to Z.

20. The antibody-drug conjugate or salt thereof of claim 1,
wherein -Q-D- has the formula: 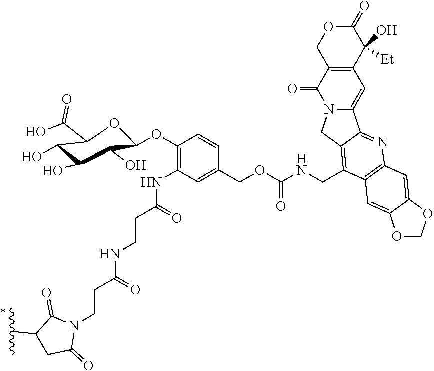
wherein
the wavy line marked with a single asterisk (*) indicates the point of covalent attachment to L.

21. The antibody-drug conjugate or salt thereof of claim 1, wherein -Q-D- has the formula: 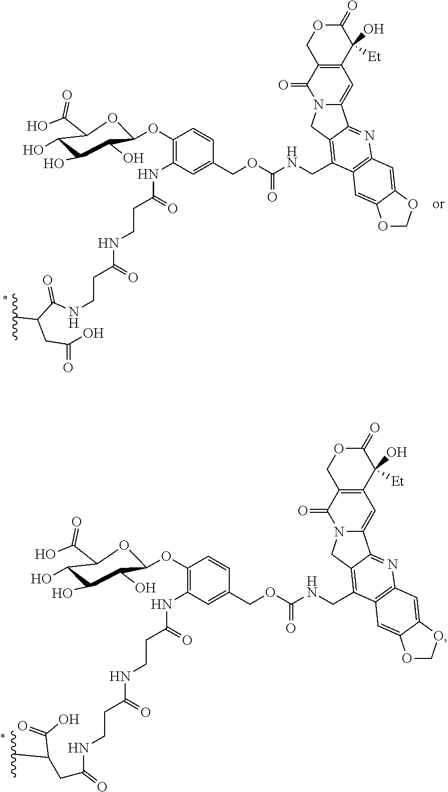
wherein
the wavy line marked with a single asterisk (*) indicates the point of covalent attachment to L.

22. The antibody-drug conjugate or salt thereof of claim 1, comprising a ratio of Drug Unit to antibody (DAR) ratio of 1 to 10.

23. The antibody-drug conjugate or salt thereof of claim 1, wherein the DAR is about 4 or about 8.

24. The antibody-drug conjugate or salt thereof of claim 2, comprising a ratio of Drug Unit to antibody (DAR) ratio of 1 to 10.

25. The antibody-drug conjugate or salt thereof of claim 2, wherein the DAR is about 4 or about 8.

26. The antibody-drug conjugate or salt thereof of claim 1, wherein the Linker Unit is attached to the antibody or antigen binding fragment at a cysteine amino acid residue.

27. An antibody-drug conjugate or salt thereof, having the formula:
L-(Q-D)₈,
wherein L comprises an antibody or antigen binding fragment thereof that binds to CEACAM5 comprising:
a heavy chain that has the amino acid sequence set forth in SEQ ID NO:9 and
a light chain that has the amino acid sequence set forth in SEQ ID NO: 10;
and wherein Q-D is

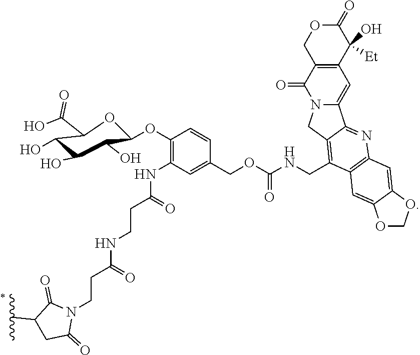

28. A pharmaceutical composition comprising the antibody-drug conjugate or salt thereof of claim 1, and a pharmaceutically acceptable carrier.

29. A method of treating cancer in an individual comprising administering the antibody-drug conjugate or salt thereof of claim 1.

30. A kit comprising the antibody-drug conjugate or salt thereof of claim 1.
